(19) United States
(12) Patent Application Publication
(10) Pub. No.: US 2026/0183405 A1
(43) Pub. Date: Jul. 2, 2026

(54) SMALL MOLECULE-DRUG-CONJUGATES CLEAVABLE IN A TUMOR MICROENVIRONMENT

(71) Applicant: Hans-Georg LERCHEN, Leverkusen (DE)

(72) Inventors: Hans-Georg LERCHEN, Leverkusen (DE); Beatrix STELTE-LUDWIG, Wülfrath (DE); Mareike WIEDMANN, Monheim am Rhein (DE); Anne-Sophie REBSTOCK, Champagne au Mont d’Or (FR); Tibor SCHOMBER, Iserlohn (DE)

(73) Assignee: Hans-Georg LERCHEN, Leverkusen (DE)

(21) Appl. No.: 19/130,666

(22) PCT Filed: Nov. 16, 2023
(86) PCT No.: PCT/EP2023/082123

§ 371 Date: May 16, 2025

(30) Foreign Application Priority Data
Nov. 17, 2022 (EP) 22306696.0
Apr. 3, 2023 (EP) 23305484.0

(51) Int. Cl. A61K 47/54; A61K 38/00; A61K 47/55; A61P 35/00

(52) U.S. Cl. CPC A61K 47/545; A61K 47/55; A61P 35/00; A61K 38/00

(57) ABSTRACT

The present invention relates to novel pharmaceutical compounds comprising one or more binding molecules (e.g., target protein binders, T) capable of binding to target proteins expressed on tumor cells or on cells present in the tumor microenvironment, and which are linked via protease cleavable linkers to one or more payload molecules; the processes for preparation thereof; and to the use thereof for treating diseases and conditions, including hyperproliferative disorders such as cancer, in humans and other mammals.

### CROSS-REFERENCE TO RELATED APPLICATIONS

This International Patent Application claims the benefit of European Patent Application No. 22306696.0, filed on Nov. 17, 2022, and of European Patent Application No. 23305484.0, filed on Apr. 3, 2023, each of which is incorporated herein by reference in its entirety.

### BACKGROUND OF THE INVENTION

This application relates to small molecule-drug conjugates (or “compounds”), pharmaceutical compositions, processes for preparation thereof, and the use thereof for treating, preventing, or managing diseases and conditions including hyperproliferative disorders, such as, cancer in humans and other mammals.

### SUMMARY

Provided herein are compounds comprising one or more active agents (i.e., payloads) having therapeutic activity (e.g., cytotoxic or immunostimulatory activity) conjugated to one or more target protein binders, preferably small molecule binders, via one or more linking units. The compounds provided herein may comprise one or more enzymatically-cleavable peptide linkers, which enable targeted release of a payload in a tumor microenvironment. The peptide linkers may, for example, be cleaved by neutrophil elastase, a protease secreted by neutrophils, particularly in response to inflammation and/or disease. Neutrophil elastase may be overexpressed in a tumor microenvironment, thereby providing a site-specific delivery of a cytotoxic payload to the tumor. Of particular advantage for use in accordance with the present invention are cell-permeable payloads, which upon release in a tumor microenvironment, may penetrate a diseased cell (e.g., a tumor cell) thereby producing cytotoxic/anti-tumor effects.

Compounds can comprise one or more (e.g., one, two, etc.) payloads, and/or one or more targeting groups, conjugated via a linker comprising a cleavable peptide, and optionally further comprising solubility-enhancing or tumor-retaining polymeric linker units (e.g., polyethylene glycol, polyethyleneimine, polysarcosine, etc.) and/or branching units. A branching unit may be a trivalent or tetravalent group, which may optionally comprise an amino acid or a peptide. Payloads provided herein include, but are not limited to, PTEFb inhibitors, topoisomerase inhibitors, kinesin spindle protein inhibitors, tubulin inhibitors, immune agonists, and the like. Specific examples include camptothecin and derivatives thereof (e.g., 7-ethyl camptothecin, exatecan, N-acetyl or N-alkyl exatecan derivatives, and the like), diterpenoids (e.g., triptolide, paclitaxel, or derivatives thereof), auristatins (e.g., monomethyl auristatin E, monomethyl auristatin F, etc.), toll-like receptor agonists (e.g., resiquimod, imiquimod), or other small molecule cytotoxins that may be cleaved by neutrophil elastase. In some embodiments, the therapeutic payload is a novel cytotoxin having favorable permeability and/or low efflux. Advantageously, it has been shown that payloads provided herein may be cleaved in a tumor microenvironment without the use of a self-immolative group, which can result in unwanted off-target release of a payload. Of additional advantage, the compounds provided herein do not require internalization within a cell in order to produce anti-tumor effects.

Compounds provided herein further comprise at least one, and optionally two, target protein binders, which further direct the compound to a desired site of action (e.g., a tumor or tumor microenvironment). The target protein binders disclosed herein are generally small molecules (having a molecular weight of about 1000 g/mol or less). The target proteins to which a binder may bind include, but are not limited to, αᵥβ₃ (avb3) integrin binders, fibroblast activation protein (FAP) binders, folate receptor (FR) binders, prostate-specific membrane antigen (PSMA) binders, carbonic anhydrase IX (CA IX or CA9) binders, and the like.

Small molecules and conjugates thereof may have certain advantages over their antibody counterparts (e.g., antibody-drug conjugates) due to their lower molecular weight, plasma protein binding, or other pharmacological or pharmacokinetic considerations. In addition, conjugates provided herein may provide additional advantages over a payload by itself, in that not only is the payload directed to a desired cell or tissue whereas the payload is non-selectively distributed throughout a subject, the conjugates provided herein may also possess novel and advantageous benefits in pharmacokinetics (e.g., distribution, metabolism, clearance, excretion, half-life, AUC, etc.). In some embodiments, conjugates provided herein have a half-life of 24 hours or more. Also provided herein are novel conjugate designs and/or arrangements of binders, payloads, and linkers. In some embodiments, a conjugate further comprises a pharmacokinetic modulator, which may modulate one or more pharmacokinetic parameters of a conjugate. In some embodiments, the pharmacokinetic modulator comprises a charged or ionizable group (e.g., a carboxylate, a sulfonate, a sulfinate, a phosphonate, an amine, an iminium, a guanidine, etc.).

In one aspect, provided herein is a compound or a pharmaceutically acceptable salt thereof, comprising one or more payloads conjugated to one or more target protein binders via a linker. In some embodiments, the compound or a pharmaceutically acceptable salt thereof has the structure of Formula (I):

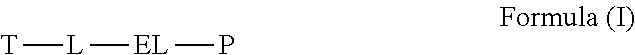

wherein:
T is a target protein binder;
P is a payload;
EL is a peptide linker, optionally further comprising a self-immolative group; and
L is a linker, optionally further comprising a second T and/or a second EL-P.
In some embodiments, provided herein is a compound or a pharmaceutically acceptable salt thereof, having a structure of Formula (II), Formula (III), Formula (IV), Formula (V), Formula (VI), Formula (VII), Formula (VIII), or Formula (IX):

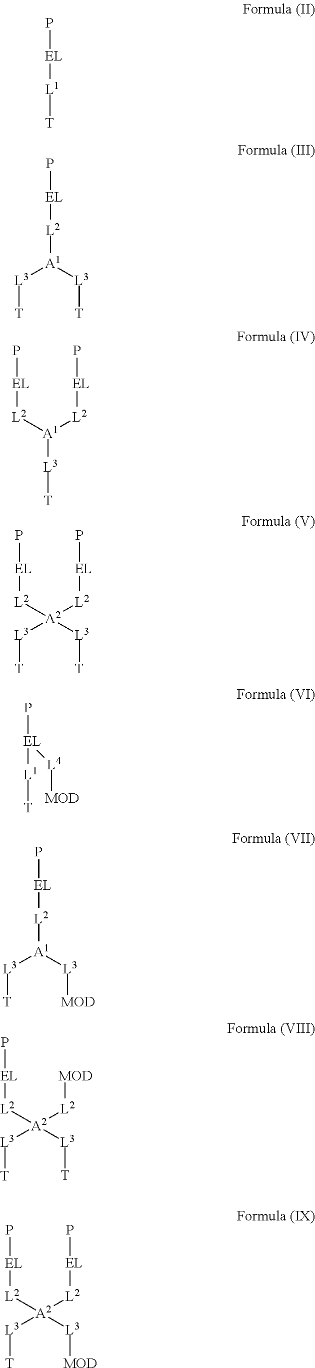

wherein:
each P is a payload;
each EL is a peptide linker, optionally further comprising a self-immolative group;
each L¹, L², L³, and L⁴ is independently a bivalent linker,
A¹ is a trivalent linker;
A² is a tetravalent linker;
each T is a target protein binder; and
MOD is a pharmacokinetic modulating group.

In some embodiments, the target protein binder is a binder of avb3 integrin, avb6 integrin, PSMA, CAIX, FAP, folate, Hsp90, somatostatin, GLUT1, APN, LRP1, bombesin, GnRH, LHRH, MT1-MMP, P32, phosphatidyl serine or sortilin. In some embodiments, the payload is a tubulin modulator, DNA modulator, RNA modulator, oxidative phosphorylation inhibitor, kinase inhibitor, Dihydrofolate reductase inhibitor, histone deacetylase inhibitor or an immunomodulator. In some embodiments, the bivalent linker comprises 1 to 12 (e.g., 3 to 6) PEG or PEI units; while a compound may comprise multiple bivalent linkers. In some embodiments, provided herein is a pharmaceutical composition comprising a compound or salt of Formula (II), Formula (III), Formula (IV), Formula (V), Formula (VI), Formula (VII), Formula (VIII), or Formula (IX), and a pharmaceutically acceptable excipient.

In still another aspect, provided herein is a compound, or a pharmaceutically acceptable salt thereof; or a stereoisomer or mixture of stereoisomers thereof, or a pharmaceutical composition thereof, for use as a medicament. In still another aspect, described herein is a compound, or a pharmaceutically acceptable salt thereof; or a stereoisomer or mixture of stereoisomers thereof, or a pharmaceutical composition thereof, for use in a method of treating a disease or disorder disclosed herein. In some embodiments, the disease or disorder is a hyperproliferative disorder. In some embodiments, the disease or disorder is a cancer.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

A better understanding of the features and advantages of the present invention will be obtained by reference to the following detailed description that sets forth illustrative embodiments, in which the principles of the invention are utilized, and the accompanying drawings (also “Figure” or “FIG.” herein), of which:

FIG. 1 shows cytokine release of freshly prepared PBMC from three healthy donors after treatment with the indicated compounds (1 μM).

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Provided here, in some embodiments, are novel pharmaceutical compounds comprising one or more binding molecules binding to target molecules expressed on tumor cells or on cells present in the tumor microenvironment and which are linked via protease cleavable linkers to one or more payload molecules, the processes for preparation thereof, and to the use thereof for treating, preventing or managing diseases and conditions including hyperproliferative disorders such as cancer in humans and other mammals.

Examples for binding molecules include binders to cell adhesion proteins like integrins such as αᵥβ₃ integrins, αᵥβ₆ integrins, prostate specific membrane antigen (PSMA), fibroblast activation protein (FAP), carbonic anhydrase IX (CAIX), a chaperone protein Heat Shock 90 (Hsp 90) binder, a Folic Acid Receptor binder, a Glucose transporter 1 binder, a Somatostatin receptor binder, an aminopeptidase N (APN) binder, a Low density lipoprotein receptor-related protein 1 (LRP1) binder, a Bombesin receptor binder, a gonadotropin releasing hormone (GnRH or LHRH) receptor binder, P32 binder, Membrane type 1 Matrix Metalloprotease (MT1-MMP) binder, a Sortilin binder, a Nectin-4 binder.

Protease cleavable linkers contain peptide sequences which are cleavable by tumor-associated enzymes present in tumor microenvironment. Many of these enzymes were shown to be part of the protease family of enzymes which are mainly involved in the modulation of the tumor stroma and the motility of tumor cells. Tumor microenvironment shaping proteases are for example serine proteases like plasmin activator, seprase, hepsin or kallikreins. Another family of protease in the TME are cystein proteases such as cathepsin B and cathepsin K or aspartyl proteases such as cathepsin D and cathepsin E, but also other proteases such as heparanase, endoglycosidase and hyaluronidase were shown to be upregulated and activated within the TME. Such enzymes can be proteases such as matrix metallo proteases, neutrophil elastase.

Payloads employed in such conjugates can be a cytotoxic or an immunostimulatory agent. In some preferred embodiments, provided herein are compounds with payloads such as camptothecin derivatives, auristatin derivatives, CDK9/PTEFb derivatives, kinesin spindle protein inhibitor derivatives, or toll-like receptor 7 and/or 8 (“TLR7/8) agonist derivatives.

Chemotherapy in cancer is accompanied by usually serious side effects which are to be attributed to the toxic action of chemotherapeutics on proliferating cells of other tissue types rather than tumor tissue. For many years, scientists have occupied themselves with the problem of improving the selectivity of active compounds employed.

20(S)-Camptothecin is a pentacyclic alkaloid which was isolated in 1966 by Wall et al. (J. Am. Chem. Soc. 88, 3888 (1966)). It has a high active antitumor potential in numerous in-vitro and in-vivo tests. Unfortunately, however, the realization of the promising potential in the clinical investigation phase failed because of toxicity and solubility problems.

A large number of camptothecin derivatives have been investigated in preclinical and clinical studies; from those, irinotecan, topotecan and belotecan have successfully been approved (Li et al, Am J Cancer Res 2017; 7(12):2350-2394). Some such derivatives (A-W) are listed below.

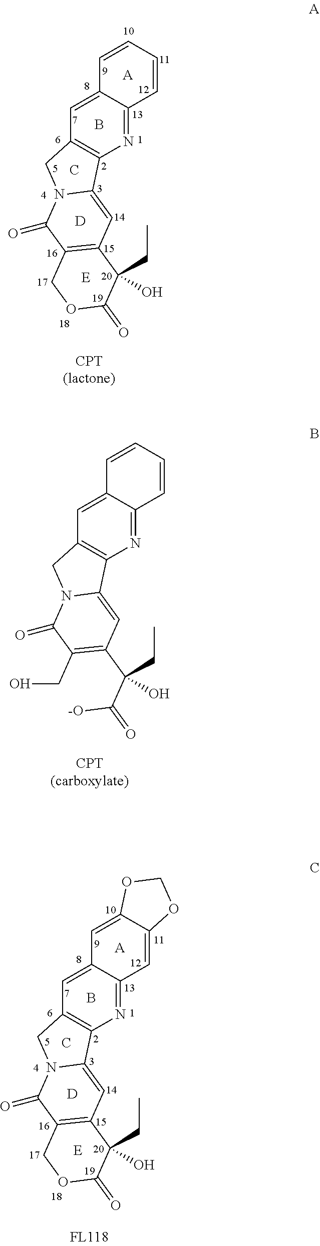

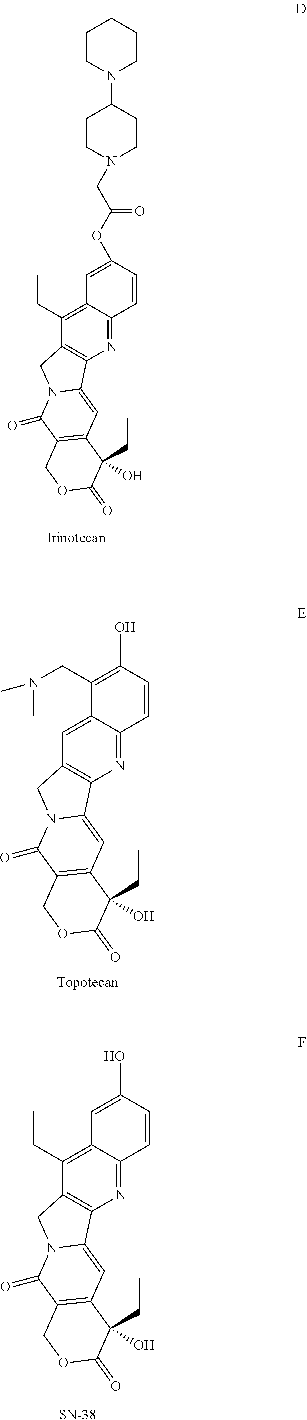 [SMILES: CCc1c2c(nc3ccc(OC(=O)CN4CCC(N5CCCCC5)CC4)cc13)-c1cc3c(c(=O)n1C2)COC(=O)[C@]3(O)CC ; CC[C@@]1(O)C(=O)OCc2c1cc1n(c2=O)Cc2cc3c(CN(C)C)c(O)ccc3nc2-1 ; CCc1c2c(nc3ccc(O)cc13)-c1cc3c(c(=O)n1C2)COC(=O)[C@]3(O)CC ; "SN-38"]

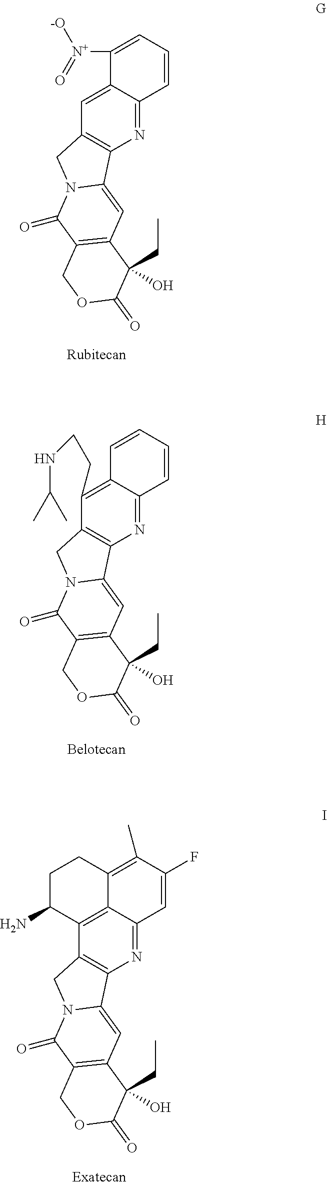

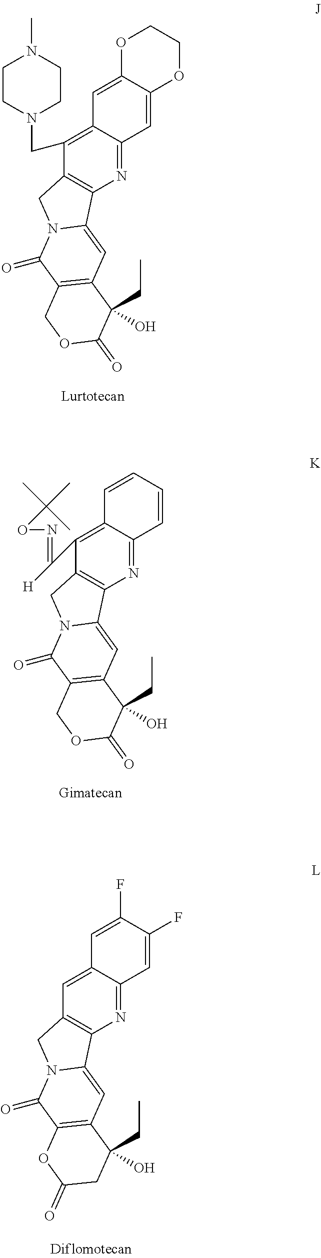 [SMILES: CC[C@@]1(O)C(=O)OCc2c1cc1n(c2=O)Cc2c-1nc1cc3c(cc1c2CN1CCN(C)CC1)OCCO3 ; CC[C@@]1(O)C(=O)OCc2c1cc1n(c2=O)Cc2c-1nc1ccccc1c2/C=N/OC(C)(C)C ; CC[C@@]1(O)CC(=O)Oc2c1cc1n(c2=O)Cc2cc3cc(F)c(F)cc3nc2-1]

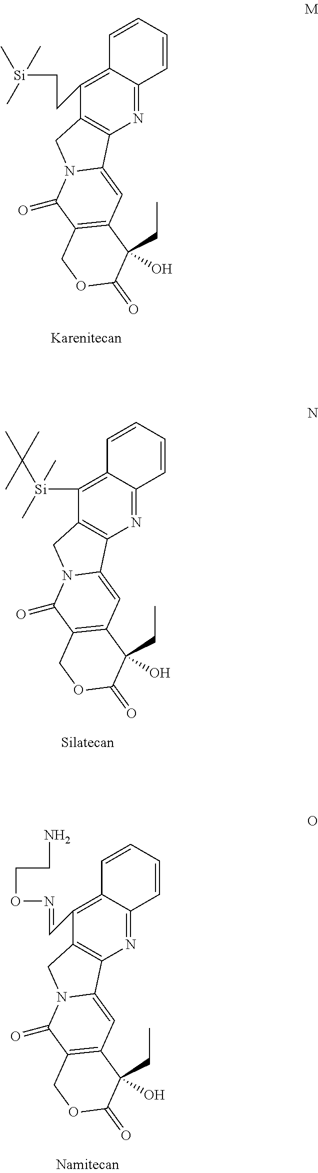 [SMILES: CC[C@@]1(O)C(=O)OCc2c1cc1n(c2=O)Cc2c-1nc1ccccc1c2CC[Si](C)(C)C ; CC[C@@]1(O)C(=O)OCc2c1cc1n(c2=O)Cc2c-1nc1ccccc1c2[Si](C)(C)C(C)(C)C ; CC[C@@]1(O)C(=O)OCc2c1cc1n(c2=O)Cc2c-1nc1ccccc1c2/C=N/OCCN]

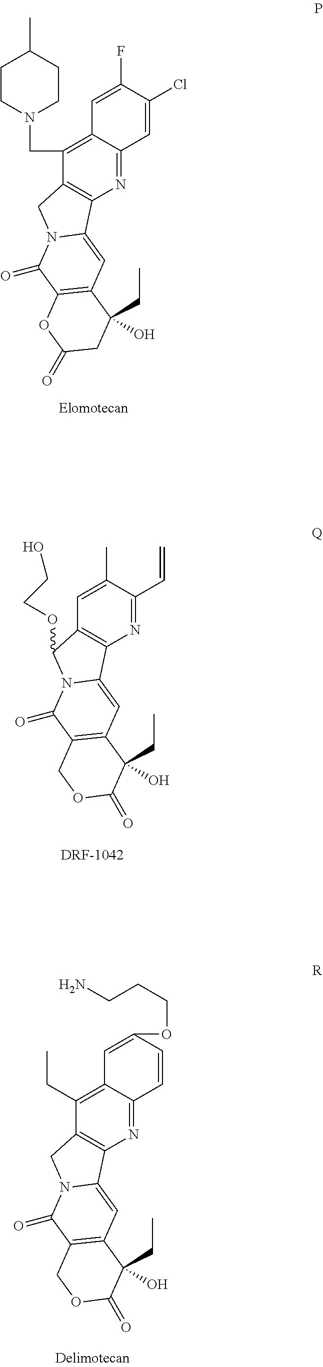 [SMILES: CC[C@@]1(O)CC(=O)Oc2c1cc1n(c2=O)Cc2c-1nc1cc(Cl)c(F)cc1c2CN1CCC(C)CC1 ; C=Cc1nc2c(cc1C)C(OCCO)n1c-2cc2c(c1=O)COC(=O)[C@]2(O)CC ; CCc1c2c(nc3ccc(OCCCN)cc13)-c1cc3c(c(=O)n1C2)COC(=O)[C@]3(O)CC]

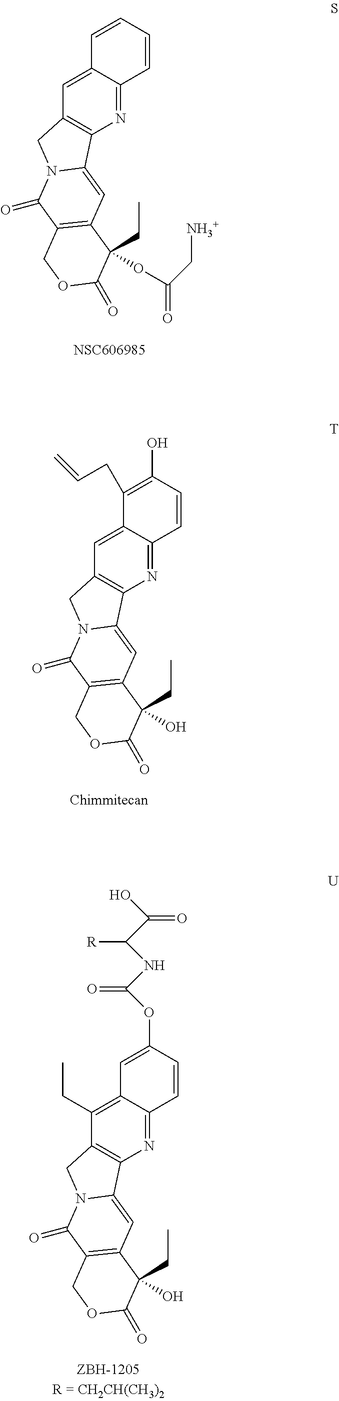

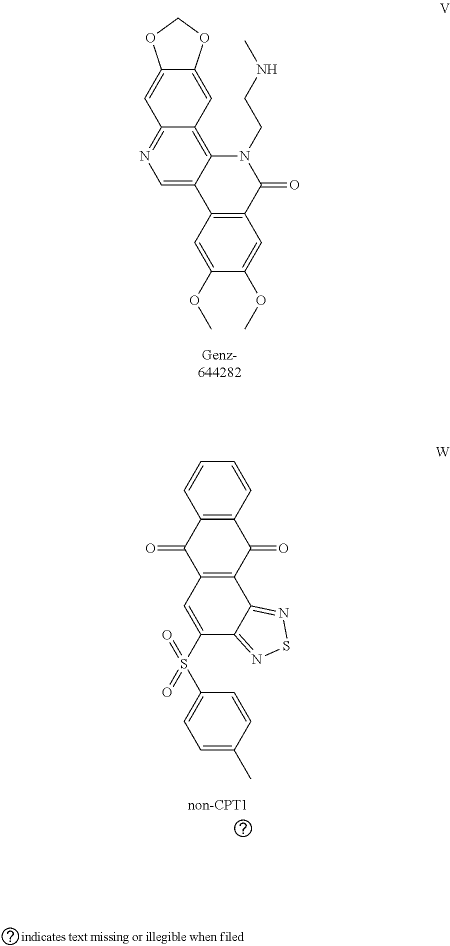 [SMILES: CNCCn1c(=O)c2cc(OC)c(OC)cc2c2cnc3cc4c(cc3c21)OCO4 ; Cc1ccc(S(=O)(=O)c2cc3c(c4nsnc24)C(=O)c2ccccc2C3=O)cc1]

Improving the therapeutic window of cytotoxic agents such as camptothecin and its derivatives remains a challenge.

Integrins are heterodimeric transmembrane proteins that may be expressed on the surface of cells, which play an important part in the adhesion of the cells to an extracellular matrix. They recognize extracellular glycoproteins such as fibronectin or vitronectin on the extracellular matrix via the RGD sequence occurring in these proteins (RGD is the single-letter code for the amino acid sequence arginine-glycine-aspartate). In general, integrins such as, for example, the vitronectin receptor, which is also called the αᵥβ₃ receptor, or alternatively the αᵥβ₅ receptor or the GpIIb/IIIa receptor play an important part in biological processes such as cell migration, angiogenesis and cell-matrix adhesion and thus for diseases in which these processes are crucial steps. Cancer, osteoporosis, arteriosclerosis, restenosis and ophthalmia may be mentioned by way of example.

The αᵥβ₃ receptor occurs, for example, in large amounts on growing endothelial cells and makes possible their adhesion to an extracellular matrix. The αᵥβ₃ receptor thus plays an important part in angiogenesis, i. e. the formation of new blood vessels, which is a crucial prerequisite for tumor growth and metastasis formation in carcinomatous disorders.

It was possible to show that the blockade of the above-mentioned receptors is an important starting point for the treatment of disorders of this type. If the adhesion of growing endothelial cells to an extracellular matrix is suppressed by blocking their corresponding integrin receptors, for example, by a cyclic peptide or a monoclonal antibody, angiogenesis does not occur, which leads to a stoppage or regression of tumour growth. In spite of compelling preclinical results demonstrating that the inhibition of integrin has therapeutic potential, clinical trials with integrin inhibitors targeting those integrins have repeatedly failed to demonstrate therapeutic benefits in cancer patients.

Conjugates provided herein can be selectively concentrated in a tumor tissue by incorporation of one or more binding molecules (i.e., target protein binders), which can bind to a target protein (e.g., receptor) that is expressed, preferably overexpressed, in tumor tissue. Conjugates provided herein may comprise one or more protease-cleavable payloads (e.g., therapeutic payloads) which, upon cleavage by a protease, exhibit cytotoxic or immune agonist properties. In order to reduce off-target cytotoxicity or immunostimulatory properties, a payload may be bound to a conjugate (as provided herein) via a protease-cleavable linker, wherein the protease is selectively expressed (e.g., overexpressed) by tumor cells, or by cells present in a tumor microenvironment. The targeted cytotoxic or immunostimulatory effects of a conjugate provided herein (or of a therapeutic payload conjugated thereto) can be further enhanced when the release of the payload takes place in the immediate vicinity of the tumor tissue (i.e., within a tumor microenvironment), or within a tumor cell. Of additional advantage and utility are therapeutic payloads which, upon release from a conjugate, can penetrate a tumor cell. Preferably, a therapeutic payload is not effluxed or transported outside of a cell.

In WO 2000/069472 enzyme-activated anti-tumor prodrug compounds are disclosed which can be specifically cleaved by collagenase (IV) and elastase. With respect to linking units cleavable by elastase this application describes that the specific tetrapeptide sequences Ala-Ala-Pro-Val and Ala-Ala-Pro-Nva are suitable. Furthermore, in this reference, no conjugates which comprise a moiety addressing αᵥβ₃ integrin receptors and a cytostatic are mentioned. Y. Liu et al. (Mol. Pharmaceutics 2012, 9, 168) describe conjugates of Auristatins linked to an αᵥβ₃ integrin targeting moiety via a legumain-cleavable linker.

In EP1238678 conjugates with cytotoxic agents are disclosed which target αᵥβ₃ integrins and have peptide linkers which can be specifically cleaved by elastase. With respect to linking units cleavable by elastase this application describes peptide sequences comprising Pro-Val and Pro-Leu. As toxophore moieties camptothecin and a quinolone carboxylic acid are exemplified.

Particular challenges of such conjugates include
sufficient solubility enabling intravenous administration in appropriate vehicles,
high tumor penetration of intact conjugates,
high stability in plasma to avoid systemic de-conjugation,
efficient binding to the targeted receptors in the tumor microenvironment,
efficient cleavage by enzymes present in the tumor microenvironment,
high cellular permeability and low efflux ratio of cleaved toxophore moieties to enhance tumor cell uptake versus re-distribution.

It is therefore an objective to develop conjugates which comprise a moiety addressing tumor targets and a payload which can be released from the conjugate preferably in tumor microenvironment, where the moiety in the conjugate addressing receptors in tumor tissue retains its ability to bind to the receptor and therefore provides tissue selectivity to such compounds. In addition, cleavability of the conjugates and drug release should be mediated by enzymes present and active in the tumor environment such as neutrophil elastase. Finally, the profile of the toxophore should be suitable for extracellular cleavage and release mechanism. The toxophore should be highly permeable into tumor cells and tissues and not be a substrate of drug transporters.

In WO2020094471 αᵥβ₃ integrin conjugates with 7-ethyl camptothecin have been described, and in WO2020094471, negatively charged carboxy groups and PEG units are incorporated into the linker to address solubility. A number of camptothecin derivatives have been investigated as a payload in Antibody-Drug-Conjugates, such as DxD in Enhertu (Modi et al, N. Engl. J. Med. 382, 610-621) and SN38 in Trodelvy (Rugo et al., Future Oncol. 16, 705-715), which both got recently approved.

Since small molecule drug conjugates may show favorable features over ADCs such as e. g. higher tumor penetration (Cazzamalli et al., J. Am. Chem. Soc. 2018, 140, 1617) the current invention comprises small molecule drug conjugates with selected camptothecin derivatives. Preferred camptothecin derivatives should show high potency, high membrane penetration properties and a low efflux ratio. Exemplary camptothecin derivatives described herein include 7-ethyl camptothecin (“7EC”), 10,11-methylenedioxy-camptothecin (“FL118”) and exatecan, or a derivative thereof (e.g., N-alkyl or N-acyl derivatives of exatecan, provided herein).

To improve tumor targeting and accumulation in the tumor microenvironment, the present invention relates to conjugates comprising one or more tumor binding molecules, linker units which can be selectively cleaved by tumor associated enzymes such as neutrophil elastase, and one or more payload molecules. Special structural features of such conjugates can be, but are not limited to a polyalkylamine spacer, multimeric binding achieved with a dendrimer approach with two or more binding molecules in the conjugate, and/or slow release prodrug residues to modulate (i) the PK of the conjugate, (ii) drug release from the conjugate, and/or (iii) the PK of the released free drug. Such conjugates have a tumor-specific action because of linkage to tumor homing molecules such as binders, via preferred linking units which can selectively be cleaved by tumor associated enzymes such as neutrophil elastase. The preferred linking units provide sufficient stability of the compound/conjugate in biological media, e.g. culture medium or serum and, at the same time, the desired intracellular action within tumor tissue as a result of its specific enzymatic or hydrolytic cleavability with release of the drug moiety.

In particular, the compounds of the present invention show one or more of the following features
Modified alkyl spacers interrupted by one or more N-alkyl amino groups with a beneficial impact on retention in tumor microenvironment having an acidic pH and furthermore allowing for high solubility
One or more tumor-binding moieties enabling high or moderate affinity
One or more payload moieties enabling high anti-proliferative potency
Despite significant steric challenges, these novel linkers appear highly susceptible for cleavage by enzymes present in tumor stroma such as neutrophil elastase
High stability of the conjugates in plasma and cytotoxic activity which is increased in the presence of elastase

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

As used herein, the term “about” a number refers to that number plus or minus 15% of that number. The term “about” a range refers to that range minus 15% of its lowest value and plus 15% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

As used herein, the term “payload” or “therapeutic payload” generally refers to a small molecule (i.e., non-protein) compound, having therapeutic (e.g., anti-cancer) activity in cells and/or tissues. Preferably, the therapeutic activity is exhibited after separation from a cleavable group. In some embodiments, the cleavable group is an enzymatically-cleavable group. In some embodiments, the therapeutic payload is activated following cleavage by a tumor-associated protein such as neutrophil elastase. A therapeutic payload may be, for example, a drug. In some embodiments, the therapeutic payload is a cytotoxic, cytostatic, or immunomodulatory compound. In some embodiments, the therapeutic payload is effective in killing or slowing the growth of cancer cells. In some embodiments, the therapeutic payload is a kinesin spindle protein inhibitor, a camptothecin or a derivative thereof, a CDK9 inhibitor, etc. as described herein.

As used herein, the term “target protein” generally refers to a protein that is expressed on the surface of a cell (e.g., a cancer cell), which can efficiently bind a small molecule binder. Payloads used herein generally refer to a compound with micromolar potency or better (e.g., sub-micromolar, nanomolar, sub-nanomolar, etc. as used in the art). Examples of target proteins as defined herein.

As used herein, the term “non-cleavable linker” refers to a linking unit of atoms (e.g., 1 to 200 atoms selected from C, H, N, O, S, and halogen) that is not known to be chemically or biologically unstable. The term “non-cleavable linker” is intended to differentiate from cleavable linkers (e.g., protease-cleavable linkers, self-immolative linkers, pH-sensitive linkers, etc.). A non-cleavable linker may be an alkyl or heteroalkyl linker, optionally interrupted by one or more cyclyl or heterocyclyl groups (e.g., click partners or artifacts therefrom). A non-cleavable linker may comprise a polymeric section (e.g., PEG, PEI, polysarcosine, etc.) and/or an alkyl section.

A non-cleavable linker may further comprise another functional group such as a small molecule target protein binder, a pharmacokinetic modulator (e.g., a —COOH group), and/or one or more therapeutic payloads. A non-cleavable may be conjoined with a protease-cleavable linker. In such an instance, the protease-cleavable linker may be cleaved by a protease (e.g., in a tumor microenvironment) to release the therapeutic payload, and leaving the non-cleavable linker stably bound to the antibody. In some embodiments, a non-cleavable linker and/or protease-cleavable linker disclosed herein does not comprise a self-immolative linker group, which may further enhance the stability (e.g., reduce off-target release) of the ADCs disclosed herein.

As used herein, C₁-Cₓ includes C₁-C₂, C₁-C₃ . . . C₁-Cₓ. By way of example only, a group designated as “C₁-C₆” indicates that there are one to six carbon atoms in the moiety, e.g., groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, e.g., the alkyl group is selected from among methyl, ethyl, propyl, iso-propyl, n-butyl, iso-butyl, sec-butyl, and t-butyl.

An “alkyl” group refers to an aliphatic hydrocarbon group. The alkyl group is branched or straight chain. In some embodiments, the “alkyl” group has 1 to 10 carbon atoms, e.g. a C₁-C₁₀alkyl. Whenever it appears herein, a numerical range such as “1 to 10” refers to each integer in the given range; e.g., “1 to 10 carbon atoms” means that the alkyl group consist of 1 carbon atom, 2 carbon atoms, 3 carbon atoms, etc., up to and including 10 carbon atoms, although the present definition also covers the occurrence of the term “alkyl” where no numerical range is designated. In some embodiments, an alkyl is a C₁-C₆alkyl. In one aspect the alkyl is methyl, ethyl, propyl, iso-propyl, n-butyl, iso-butyl, sec-butyl, or t-butyl. Typical alkyl groups include, but are in no way limited to, methyl, ethyl, propyl, isopropyl, butyl, isobutyl, sec-butyl, tertiary butyl, pentyl, neopentyl, or hexyl.

An “alkylene” group refers to a divalent alkyl radical. Any of the above mentioned monovalent alkyl groups may be an alkylene by abstraction of a second hydrogen atom from the alkyl. In some embodiments, an alkylene is a C₁-C₆alkylene. In other embodiments, an alkylene is a C₁-C₄alkylene. Typical alkylene groups include, but not limited to, —CH₂—, —CH₂CH₂—, —CH₂CH₂CH₂—, —CH₂CH₂CH₂CH₂—, and the like. In some embodiments, an alkylene is —CH₂—.

An “alkoxy” group refers to a —O(alkyl) group, where alkyl is as defined herein. Examples of alkoxy groups include —OCH₃, —OCH₂CH₃, —OCH₂CH₂CH₃, —OC(CH₃)₃, and the like.

An “hydroxyalkyl” refers to an alkyl in which one hydrogen atom is replaced by a hydroxyl. In some embodiments, a hydroxyalkyl is a C₁-C₄hydroxyalkyl. Typical hydroxyalkyl groups include, but not limited to, —CH₂OH, —CH₂CH₂OH, —CH₂CH₂CH₂OH, —CH₂CH₂CH₂CH₂OH, —C(CH₃)₂OH, and the like.

The term “alkylamine” refers to the —N(alkyl)ₓH_{y} group, where x is 0 and y is 2, or where x is 1 and y is 1, or where x is 2 and y is 0.

An “aminoalkyl” refers to an alkyl in which one hydrogen atom is replaced by an amino. In some embodiments, aminoalkyl is a C₁-C₄aminoalkyl. Typical aminoalkyl groups include, but not limited to, —CH₂NH₂, —CH₂CH₂NH₂, —CH₂CH₂CH₂NH₂, —CH₂CH₂CH₂CH₂NH₂, —C(CH₃)₂NH₂, and the like.

The term, “heteroalkyl” generally refers to a straight-chain and/or branched hydrocarbon chain which has 1 to 30 carbon atoms and may be interrupted once or more than once by one or more of the groups —O—, —S—, —C(═O)—, —S(═O)—, —S(═O)₂—, —NR^{y}—, —NR^{y}C(═O)—, —C(═O)—NR^{y}—, —NR^{y}NR^{y}—, —S(═O)₂—NR^{y}NR^{y}—, —C(═O)—NR^{y}NR^{y}—, —CR^{x}═N—O—, and where the hydrocarbon chain including the side chains, if present, may be substituted by —NH—C(═O)—NH₂, —C(═O)—OH, —OH, —NH₂, —NH—C(═NNH₂)—, sulfonamide, sulfone, sulfoxide, sulfonic acid, sulfamide, or a combination thereof. In this context, R^{y} in each case is —H, phenyl, C₁-C₁₀-alkyl, C₂-C₁₀-alkenyl or C₂-C₁₀-alkynyl, which may in turn be substituted in each case by —NHC(O)NH₂, —COOH, —OH, —NH₂, —NH—C(═NNH₂)—, sulfonamide, sulfone, sulfoxide, sulfonic acid, sulfamide, or a combination thereof. In this context, R^{x} is —H, C₁-C₃-alkyl or phenyl. The terms “heteroalkyl-aryl” and “heteroalkyl-heteroaryl” as used herein generally refer to a heteroalkyl group (as defined above) substituted with an aromatic carbocycle or an aromatic heterocycle respectively; and wherein each is optionally substituted.

The term “aromatic” refers to a planar ring having a delocalized n-electron system containing 4n+2 π electrons, where n is an integer. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (or rings which share adjacent pairs of carbon atoms) groups.

The term “carbocyclic” or “carbocycle” refers to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic. Carbocycles include aryls and cycloalkyls.

As used herein, the term “aryl” refers to an aromatic ring wherein each of the atoms forming the ring is a carbon atom. In one aspect, aryl is phenyl or a naphthyl. In some embodiments, an aryl is a phenyl. In some embodiments, an aryl is a phenyl, naphthyl, indanyl, indenyl, or tetrahydronaphthyl. In some embodiments, an aryl is a C₆-C₁₀aryl. Depending on the structure, an aryl group is a monoradical or a diradical (or an arylene group). As used herein, the term “aralkyl” generally refers to a monocyclic aromatic carbocycle (e.g., phenyl), to which a C₁₋₄-alkyl group is bonded. Illustrative aralkyl groups include benzyl and ethylphenyl.

The term “cycloalkyl” refers to a monocyclic or polycyclic aliphatic, non-aromatic radical, wherein each of the atoms forming the ring (or skeletal atoms) is a carbon atom. In some embodiments, cycloalkyls are spirocyclic or bridged compounds. In some embodiments, cycloalkyls are optionally fused with an aromatic ring, and the point of attachment is at a carbon that is not an aromatic ring carbon atom. Cycloalkyl groups include groups having from 3 to 10 ring atoms. In some embodiments, cycloalkyl groups are selected from among cyclopropyl, cyclobutyl, cyclopentyl, cyclopentenyl, cyclohexyl, cyclohexenyl, cycloheptyl, cyclooctyl, spiro[2.2]pentyl, norbornyl and bicycle[1.1.1]pentyl. In some embodiments, a cycloalkyl is a C₃-C₆cycloalkyl. In some embodiments, a cycloalkyl is a C₃-C₄cycloalkyl.

The term “halo” or, alternatively, “halogen” or “halide” means fluoro, chloro, bromo or iodo. In some embodiments, halo is fluoro, chloro, or bromo.

The term “fluoroalkyl” refers to an alkyl in which one or more hydrogen atoms are replaced by a fluorine atom. In one aspect, a fluoroalkyl is a C₁-C₆fluoroalkyl.

The term “optionally substituted” or “substituted” means that the referenced group is optionally substituted with one or more additional group(s) individually and independently selected from halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from halogen, —CN, —NH₂, —OH, —NH(CH₃), —N(CH₃)₂, —CH₃, —CH₂CH₃, —CHF₂, —CF₃, —OCH₃, —OCHF₂, and —OCF₃. In some embodiments, substituted groups are substituted with one or two of the preceding groups. In some embodiments, an optional substituent on an aliphatic carbon atom (acyclic or cyclic) includes oxo (═O).

“Small molecule,” as used herein, generally refers to any molecule having a molecular weight of about 1000 atomic mass units (Daltons) or less. In some embodiments, a moiety within a compound described herein is referred to as a small molecule, meaning that moiety has a molecular weight of about 1000 Da or less. Small molecules, as used herein, excludes proteins or antibodies, but may comprise peptides or amino acids. In some instances, a compound described herein is a conjugate of two small molecule moieties. Therefore, compounds described herein may be referred to as “small molecule drug conjugates” (SMDCs) or “small molecule prodrug conjugates” (SMPCs). For example, a SMPC may contain a cleavable group (e.g., enzymatically or chemically cleavable) such as an ester which, upon cleavage, results in a SMDC. In some examples, an SMPC (sometimes referred to as a prodrug) described herein can first be cleaved (e.g., in plasma) before an enzyme can efficiently recognize and cleave the SMDC, thus liberating the active agent(s) in two steps. In some embodiments, a SMPC enables slow conversion to the SMDC, which is cleaved more rapidly (i.e., in the presence of a suitable enzyme (e.g., a tumor associated enzyme such as elastase, legumain, or cathepsin)), thus enhancing the therapeutic window or reducing side effects.

“Pharmaceutically acceptable,” as used herein, refers a material, such as a carrier or diluent, which does not abrogate the biological activity or properties of the compound, and is relatively nontoxic at the concentration or amount used, e.g., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zürich: Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible and this capability can be manipulated as one aspect of delayed and sustained release behaviors. Also, because the salt-forming molecule can be in equilibrium with a neutral form, passage through biological membranes can be adjusted.

The term “acceptable” with respect to a formulation, composition or ingredient, as used herein, means having no persistent detrimental effect on the general health of the subject being treated.

The terms “administer,” “administering”, “administration,” and the like, as used herein, refer to the methods that may be used to enable delivery of compounds or compositions to the desired site of biological action. These methods include, but not limited to, oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally.

The terms “effective amount” or “therapeutically effective amount,” as used herein, refer to a sufficient amount of an agent or a compound being administered, which will relieve to some extent one or more of the symptoms of the disease or condition being treated. The result includes reduction or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The section headings used herein are for organizational purposes only and are not to be construed as limiting the subject matter described.

### Small Molecule-Drug Conjugates

Cancer cells or tumor microenvironment overexpress certain enzymes, including, but not limited to neutrophil elastase. Conjugates described herein are generally enzymatically cleavable by neutrophil elastase. In some embodiments, conjugates described herein are selectively cleavable in the microenvironment of cancer cells, with less cleavage taking place in the circulation or in healthy tissues. In some embodiments, a conjugate described herein is cytotoxic or immunostimulatory after activation by a tumor-associated enzyme, such as, neutrophil elastase, and/or legumain. In some embodiments, a conjugate described herein is non-toxic, in therapeutic concentrations, in the absence of said activation. The cytotoxic agents described herein are inhibitors of topoisomerase (topoisomerase I), cyclin-dependent kinase 9 (CDK9)/positive transcription elongation factor (P-TEFb), kinesin spindle protein (KSP), or of tubulin polymerization. In some embodiments, the cytotoxic agent is a small molecule, a peptide, a peptide derivative (e.g., an auristatin). In some embodiments, the conjugates described herein are immunostimulatory following activation by neutrophil elastase (e.g., via activation of toll-like receptors (TLR), which may include TLR 7 and/or TLR 8 (TLR7/8)). The cytotoxic or immunostimulatory agent may also be conjugated, e.g., via an enzymatically cleavable linker or a spacer, to a target protein binding moiety, or to a polyvalent linker to two or more target protein binders. In some embodiments, the target protein is αᵥβ₃ (avb3) integrin, αᵥβ₆ (avb6) integrin, fibroblast activation protein (FAP), folate receptor (FR), prostate-specific membrane antigen (PSMA), or carbonic anhydrase IX (CA IX or CA9).

In some embodiments, provided herein are small molecule-drug conjugates (SMDC) which have been designed for payload release and activation in the tumor microenvironment (TME). The linkers disclosed herein have been optimized to be substrate sequences for enzymes which are upregulated in the tumor microenvironment. Many of these enzymes are part of the protease family of enzymes which may be involved in the modulation of the tumor stroma and the motility of tumor cells. Some of these tumor microenvironment shaping proteases are, for example, serine proteases like plasmin activator, seprase, hepsin or kallikreins. Another family of protease in the TME are cysteine proteases such as cathepsin B and cathepsin K or aspartyl proteases such as cathepsin D and cathepsin E, but also other proteases such as heparanase, endoglycosidase and hyaluronidase were shown to be upregulated and activated within the TME. Such enzymes can be proteases such as matrix metalloproteases cathepsins and neutrophil elastase.

Another key aspect for SMDC performance is the physicochemical profile of the payload which is released. Payloads which are extracellularly cleaved in TME from the SMDCs disclosed in the current invention should be membrane permeable and efficiently penetrate into tumor tissue. The release of cell-permeable payloads is associated with a bystander killing effect, which is considered to be particularly beneficial for the treatment of tumors with heterogenous target expression.

Disclosed herein are conjugates comprising one or more small molecule target protein binders linked via a linker, and one or more therapeutic payloads linked via a protease-cleavable linker. The linker may further comprise a non-cleavable linker. Non-cleavable linkers described herein may be bivalent or multivalent. For example, in some embodiments, provided herein is a conjugate comprising a non-cleavable linker which conjoins a target protein binder to multiple therapeutic payloads, wherein each therapeutic payload is linked to the non-cleavable linker via a protease-cleavable linker. In other embodiments, the non-cleavable linker conjoins a single target protein binder to a single therapeutic payload, though a given conjugate may comprise multiple non-cleavable linkers each conjoined to a therapeutic payload via a protease-cleavable linker. Non-cleavable linkers may comprise functional elements, including physicochemical-modulating elements (e.g., solubility enhancers), pharmacokinetic-modulating elements (e.g., tumor-targeting or tumor-retained groups), proximity-modulating elements (e.g., spacers), or any combination thereof. In some embodiments, a non-cleavable linker disclosed herein (e.g., L¹, L²), is conjoined to a protease-cleavable linker (e.g., P¹, P^{1a}).

Therapeutic payloads for use in accordance with the present invention may be, for example, a drug. Thus, conjugates are generally referred to herein as SMDCs. However, the term SMDC as used herein may refer to any conjugate comprising a target protein binder and a therapeutic payload and is not limited specifically to therapeutic payload that is necessarily defined as a “drug.” A therapeutic payload can be, for example, a cytotoxic agent or an immunostimulatory agent. Preferably, the therapeutic payload is permeable to cell membranes (e.g., to tumor cell membranes). More preferably, the therapeutic payload is penetrant to tumor cells and produces cytotoxic or antiproliferative effects in a cell. A therapeutic payload may be configured to be released extracellularly in a tumor microenvironment (e.g., by via cleavage of a protease-cleavable linker by an extracellular tumor-associated protein (e.g., neutrophil elastase)). An extracellularly released therapeutic payload, e.g., a tumor-penetrant therapeutic payload, may enter a tumor cell and produce potent cytotoxic or immunostimulant effects. In some instances, the therapeutic payload is a microtubule toxin, DNA toxin, transcription toxin, or an immune stimulator. In some instances, the microtubule toxin is a maytansinoid, auristatin, epithilone, taxoid, tubulysin, eribulin alkaloid, vinca alkaloid, eribulin, or any combination thereof. In some instances, the DNA toxin is an anthracycline, topoisomerase I inhibitor, duacarmycin or analogs thereof, calichearmicins, DNA cross linking agents, bleomycin A2, dactinomucin, mitomycin C, or any combination thereof. In some instances, the transcription toxin is an amatoxin, thailanstatin A, oxidating phosphorylation inhibitor, protein kinase inhibitor, dihydrofolate reductase (DHFR) inhibitor, or histone deactylase inhibitor.

The present invention may further comprise one or more small molecule binders (SMBs) of a target protein. The target protein is, in some instances, a target protein on a tumor cell. In some embodiments, the target protein is selected from alpha-v beta-3 (“αᵥß₃” or “avß3”) integrin, alpha-v beta-6 (“αᵥß₆” or “avß6”) integrin, carbonic anhydrase IX (“CA9” or “CAIX”), fibroblast activating protein (“FAP”), prostate specific membrane antigen (“PSMA”), heat shock protein 90 (“Hsp 90”), folic acid receptor, glucose transporter 1 (GLUT1), somatostatin receptor, aminopeptidase N (APN), low density lipoprotein receptor-related protein 1 (LRP1), bombesin receptor, gonadotropin releasing hormone (GnRH) receptor, luteinizing hormone-releasing hormone (LHRH) receptor, p32, membrane type 1 matrix metalloprotease (MT1-MMP), Sortilin, or Nectin-4. Other target proteins are B-lymphocyte antigen CD20 (“CD20”), complement receptor type 2 (“CD21”), Lyb-2 (“CD72”), programmed cell death ligand 1 (“PD-L1”), carcinoembryonic antigen cell adhesion molecule (“CEACAM5”), galectin-3-binding protein (“Gal-3-BP”), leucine-rich alpha-2-glycoprotein 1 (“LRG1”), matrix metallopeptidase 9 (“MMP9”), tumour-associated glycoprotein 72 (“TAG72”), fibronectin 1 (“FN1”), tenascin-C (“TN-C”), collagen type XI alpha 1 chain (“COL11A1”), Collagen type XII alpha 1 chain (“COL12A1”), collagen type I alpha 1 chain (“COL1A1”), collagen type I alpha 2 chain (“COL1A2”), collagen type III alpha 1 chain (“COL3A1”), collagen type V alpha 1 chain (“COL5A1”), collagen type V alpha chain 2 (“COL5A2”), collagen type VI alpha 3 chain (“COL6A3”), collagen type VIII alpha 1 chain (“COL8A1”), inhibin subunit beta A (“INHBA”), periostin (“POSTN”), thrombospondin 2 (“THBS2”), zinc-alpha-2-glxcoprotein (“AZGP1”), AE binding protein 1 (“AEBP1”), anterior gradient 3 (“AGR3”), asporin (“ASPN”), BMP/retinoic acid inducible neural specific 3 (“BRINP3”), chitinase 3 like 1 (“CHI3L1”), cartilage intermediate layer protein (“CILP”), collagen type X alpha 1 chain (“COL10A1”), cartilage oligomeric matrix protein (“COMP”), cystatin SN (“CST1”), collagen triple helix repeat containing 1 (“CTHRC1”), epiphycan (“EPYC”), follicular dendritic cell secreted protein (“FDCSP”), gastrin releasing peptide (“GRP”), integrin binding sialoprotein (“IBSP”), interleukin 4 induced 1 (“IL4I1”), lumican (“LUM”), matrilin 3 (“MATN3”), midkine (“MDK”), microfibril associated protein 2 (“MFAP2”), matrix Gla protein (“MGP”), matrix metallopeptidase 1 (“MMP1”), matrix metallopeptidase 11 (“MMP11”), matrix metallopeptidase 12 (“MMP12”), matrix metallopeptidase 13 (“MMP13”), matrix metallopeptidase 3 (“MMP3”), matrix metallopeptidase 7 (“MMP7”), mucin like 1 (“MUCL1”), matrix remodeling associated 5 (“MXRA5”), signal peptide CUB domain and EGF like domain containing 2 (“SCUBE2”), secreted frizzled related protein 4 (“SFRP4”), stanniocalcin 2 (“STC2”) and zymogen granule protein 16B (“ZG16B”). Further examples of relevant target proteins can be found in F. Fauteux, Oncotarget, 2015, 7, 3, 2555. and in N. Ashman, Chem Soc Rev, 2022, Advance Article.

In some embodiments, provided herein is a compound or a pharmaceutically acceptable salt thereof, comprising one or more payloads conjugated to one or more target protein binders via a linker. In some embodiments, provided herein is a compound or a pharmaceutically acceptable salt thereof, having a structure of Formula (I):

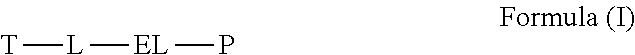

wherein:
T is a target protein binder;
L is a linker, optionally further comprising a second T, a second EL-P, and/or a pharmacokinetic modulating group (MOD);
EL is a peptide linker, and
P is a payload. In some embodiments, EL and P are conjoined via a self-immolative linker group. In some embodiments, EL and P are conjoined without a self-immolative linker.

In some embodiments, provided herein is a compound or a pharmaceutically acceptable salt thereof, having a structure of Formula (II), Formula (III), Formula (IV), Formula V Formula VI Formula (VII), Formula VIII or Formula IX):

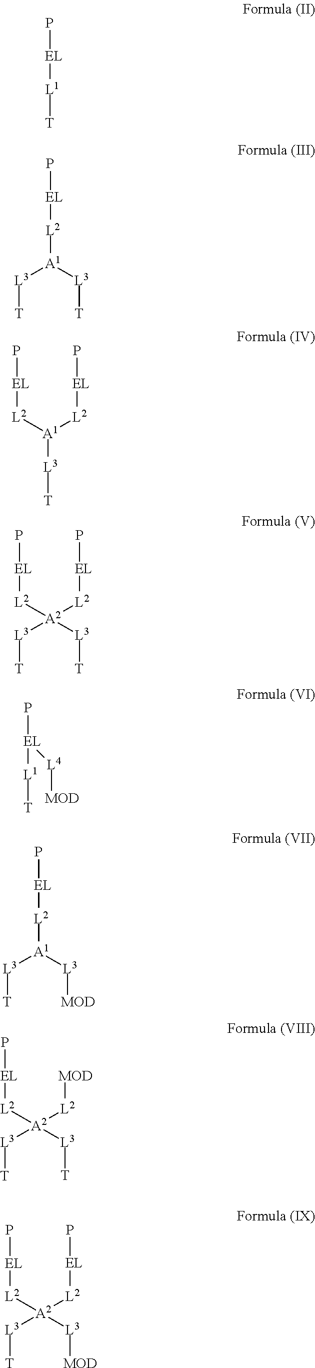

wherein:
P is a payload;
EL is a cleavable peptide linker, optionally further comprising a self-immolative group;
each L¹, L², L³, and L⁴ is independently a bivalent linker,
A¹ is a trivalent linker;
A² is a tetravalent linker;
T is a target protein binder; and
MOD is a pharmacokinetic modulating group.

In some embodiments, each target protein is independently selected from the group consisting of: integrin (e.g., alpha-v-beta-3), integrin (e.g., alpha-v-beta-6), PSMA, CAIX, FAP, folate, Hsp90, somatostatin, GLUT1, APN, LRP1, bombesin, GnRH, LHRH, MT1-MMP, P32, phosphatidyl serine or sortilin.

In some embodiments, the target protein binder is an integrin (e.g., alpha-v-beta-3 integrin) binder, integrin (e.g., alpha-v-beta-6 integrin) binder, PSMA binder, CAIX binder, FAP binder, folate receptor (FR) binder, Hsp90 binder, somatostatin binder, GLUT1 binder, APN binder, LRP1 binder, bombesin binder, GnRH binder, LHRH binder, MT1-MMP binder, P32 binder, phosphatidyl serine binder or sortilin binder. In some embodiments, the target protein binder (T) is a PSMA binder, a small molecule αᵥβ₃ integrin binder, a CAIX (CA9) binder, a FAP binder, a folate binder, or a Hsp90 binder. In some embodiments, the target protein binder (T) is a small molecule 43 (alpha-v-beta-3) integrin binder. In some embodiments, the target protein binder (T) is a FAP binder. In some embodiments, the target protein binder (T) is a PSMA binder. In some embodiments, the target protein binder (T) is a CAIX (CA9) binder. In some embodiments, the target protein binder (T) is a folate receptor (FR) binder. In some embodiments, the target protein binder (T) is a Hsp90 binder. In some embodiments, the target protein binder (T) is a PSMA binder, CAIX (CA9) binder, a FAP binder, a folate binder, or a Hsp90 binder.

In some embodiments, the target protein binder (T) of any one of formulae I, II, III, IV, V, VI, VII, VIII, or IX is:

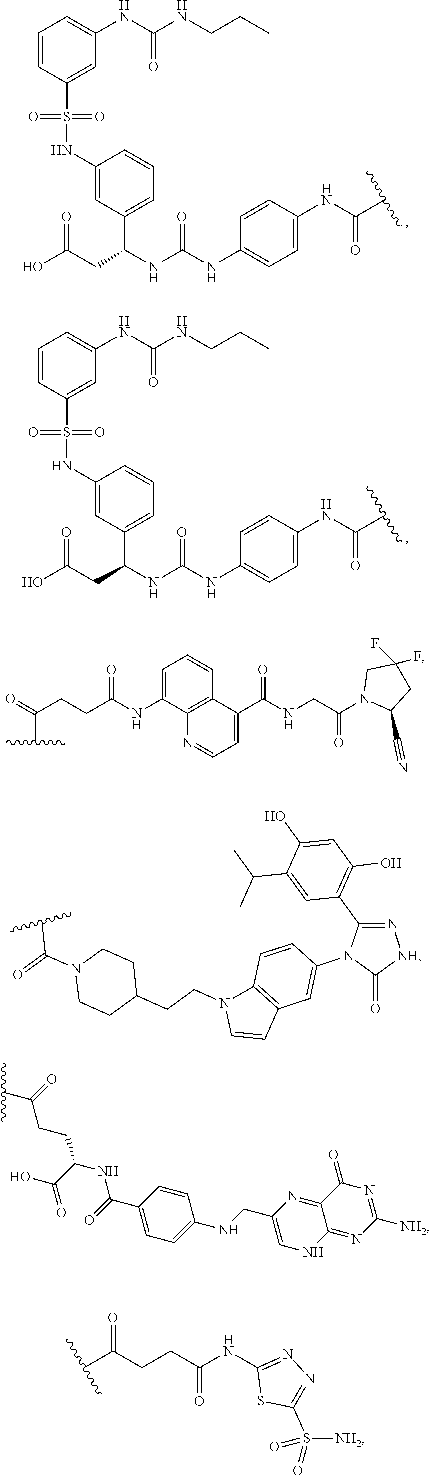 [SMILES: *C(=O)Nc1ccc(NC(=O)N[C@H](CC(=O)O)c2cccc(NS(=O)(=O)c3cccc(NC(=O)NCCC)c3)c2)cc1 ; *C(=O)Nc1ccc(NC(=O)N[C@@H](CC(=O)O)c2cccc(NS(=O)(=O)c3cccc(NC(=O)NCCC)c3)c2)cc1 ; *C(=O)CCC(=O)Nc1cccc2c(C(=O)NCC(=O)N3CC(F)(F)C[C@H]3C#N)ccnc12 ; *C(=O)N1CCC(CCn2ccc3cc(-n4c(-c5cc(C(C)C)c(O)cc5O)n[nH]c4=O)ccc32)CC1 ; *C(=O)CC[C@H](NC(=O)c1ccc(NCc2c[nH]c3nc(N)nc(=O)c-3n2)cc1)C(=O)O ; *C(=O)CCC(=O)Nc1nnc(S(N)(=O)=O)s1]

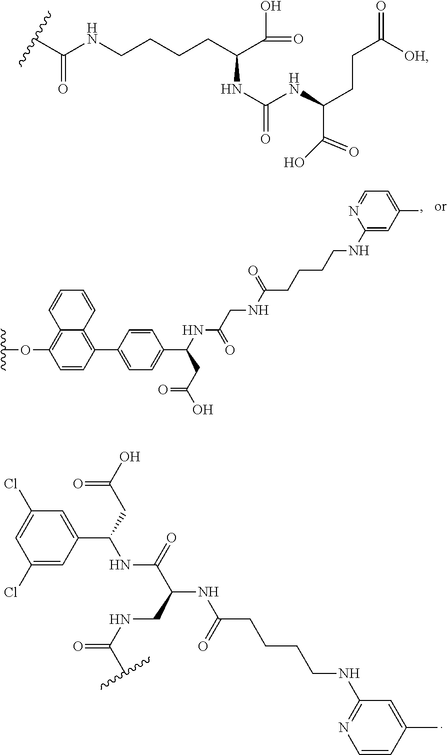

In some embodiments, P is a cytotoxic drug or immunostimulatory agent. In some embodiments, P is a cytotoxic compound. In some embodiments, P is an immunostimulatory agent. In some embodiments, P is a topoisomerase inhibitor, kinesin spindle protein inhibitor, cyclin dependent kinase 9 inhibitor, tubulin inhibitor, epidermal growth factor receptor inhibitor, a taxane, a diterpenoid, or an agonist of toll like receptor 7 and/or 8. In some embodiments, P is a cell-permeable topoisomerase inhibitor, a cell-permeable kinesin spindle protein inhibitor, a cell-permeable cyclin dependent kinase 9 inhibitor, a cell-permeable tubulin inhibitor, or a cell-permeable agonist of toll like receptor 7 and/or 8. In some embodiments, P is a topoisomerase inhibitor provided herein. In some embodiments, P is a CDK9 (e.g., PTEFb) inhibitor provided herein. In some embodiments, D¹ is an auristatin (e.g., auristatin E or monomethyl auristatin E). In some embodiments, P is a cell-permeable kinesin spindle protein inhibitor. In some embodiments, P is a toll-like receptor (e.g., TLR 7 and/or TLR8) agonist (e.g., resiquimod). In some embodiments, P is biologically active (i.e., cytotoxic or immunostimulatory) following activation by a protease such as neutrophil elastase. In some embodiments, P is not biologically active until activation via cleavage by neutrophil elastase.

In some embodiments, a payload (P) is selected from the group consisting of:

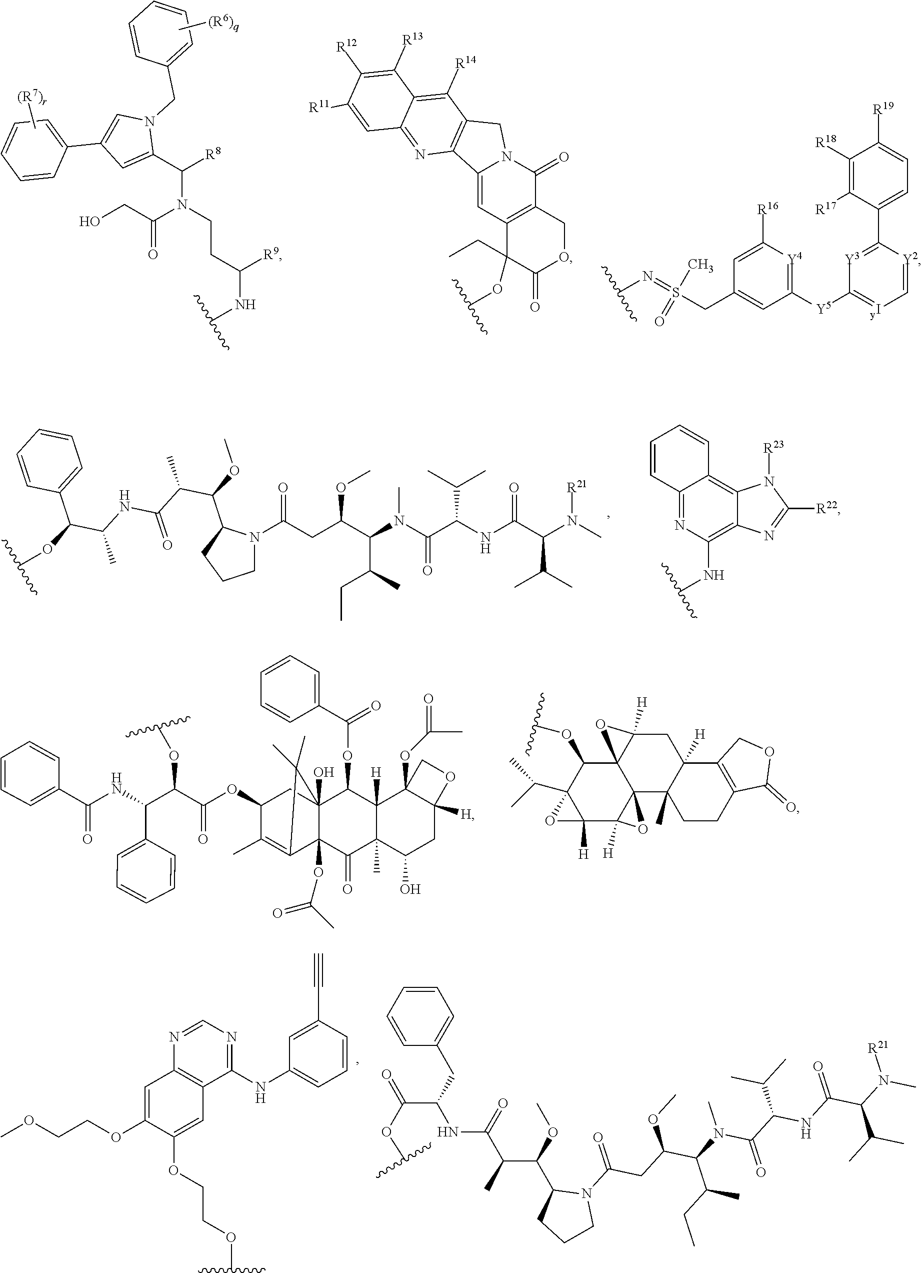

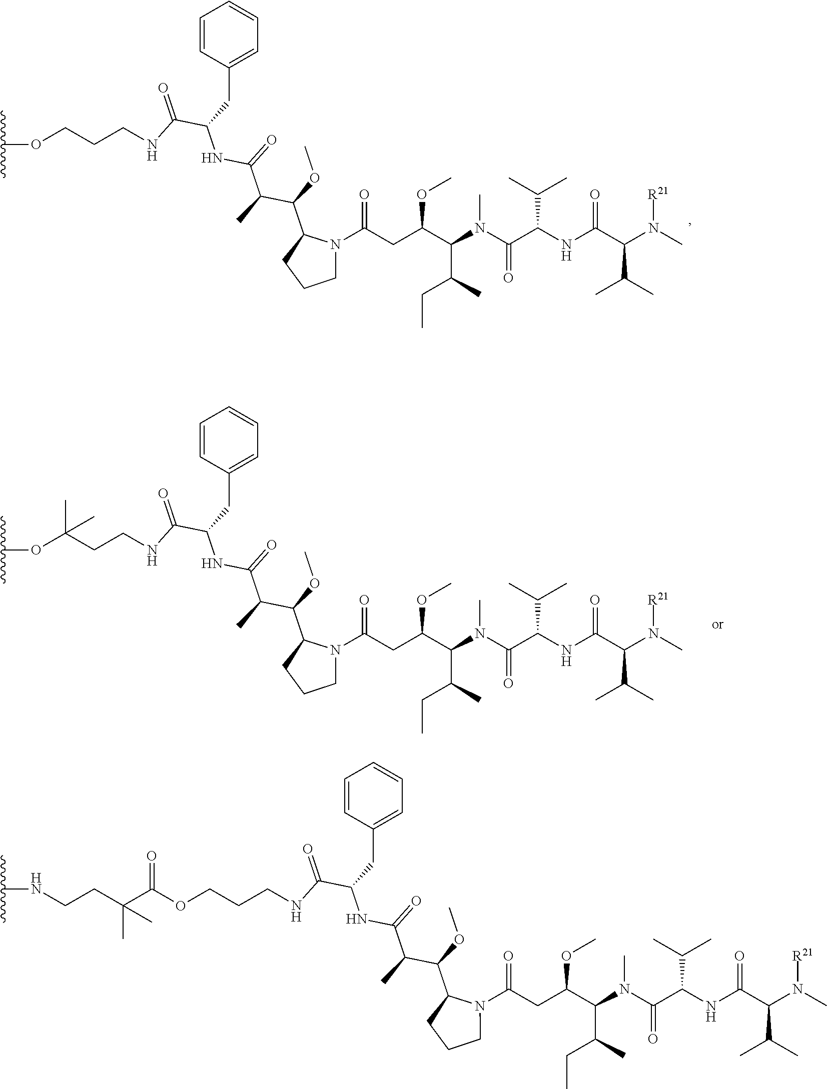

or a pharmaceutically acceptable salt thereof, wherein:
each R⁶ and R⁷ is independently hydrogen, halogen, CN, —C₁₋₆ alkyl, or C₁₋₆ haloalkyl;
R⁸ is hydrogen, halogen, CN, —C₁₋₆ alkyl, C₁₋₆ haloalkyl, C₃₋₆ cycloalkyl, or 5- to 7-membered heterocycloalkyl;
R⁹ is hydrogen, halogen, CN, C₁₋₆ alkyl, —C(O)NH₂, —C(O)NHC₁₋₆ alkyl, —C(O)N(C₁₋₆ alkyl)₂, —C(O)NHC₁₋₆ alkyl-C(O)NHC₁₋₆ alkyl, —C(O)NHC₁₋₆ alkyl-NHC(O)C₁₋₆ alkyl, —NH₂, —NHC₁₋₆ alkyl, —N(C₁₋₆ alkyl)₂, —NHC(O)C₁₋₆ alkyl, —OH, or —OC₁₋₆ alkyl; wherein each C₁₋₆ alkyl is substituted with 0-5 R¹⁰;
R¹⁰ is in each instance independently selected from the group consisting of hydrogen, halogen, CN, —COOH, —CONH₂, —NH₂, —NHCH₃, —N(CH₃)₂, —OH, and —OCH₃;
R¹¹ and R¹² are each independently hydrogen, halogen, C₁₋₆ alkyl, C₁₋₆ haloalkyl, or —OH; or R¹¹ and R¹², taken together, form a 5- or 6-membered heterocycle;
R¹³ and R¹⁴ are each independently hydrogen, C₁₋₆ alkyl, or C₁₋₆ alkylamine; or R¹³ and R¹⁴, taken together, form a C₆ carbocycle substituted with —N(R¹⁵)₂;
each R¹⁵ is independently hydrogen, C₁₋₆ alkyl, —C(O)C₁₋₆ alkyl, —C(O)NHC₁₋₆ alkyl or —C(O)OC₁₋₆ alkyl; wherein the C₁₋₆ alkyl of R¹⁵ is optionally substituted with halogen, hydroxy, phenyl, or heteroaryl; or R¹⁵ is a cleavable prodrug group;
R¹⁶ is hydrogen, halogen, C₁₋₆ alkyl, C₁₋₆ haloalkyl, —OH, —OCH₃, or —OCF₃;
R¹⁷ is hydrogen, halogen, C₁₋₆ alkyl, C₁₋₆ haloalkyl, —OH, —OCH₃, or —OCF₃; or R⁶ and R⁴, taken together, form a heteroalkylene group of the formula: —O—C₂₋₁₀ alkylene-O—, —NH—C₂₋₁₀ alkylene-O—, or —NH—C₂₋₁₀ alkylene-NH—; wherein the heteroalkylene group is optionally substituted with R²⁰;
R¹⁸ is hydrogen, halogen, C₁₋₆ alkyl, C₁₋₆ haloalkyl, —OH, —OCH₃, or —OCF₃; or R¹⁶ and R¹⁸, taken together, form a heteroalkylene group of the formula: —O—C₂₋₁₀ alkylene-O—, —NH—C₂₋₁₀ alkylene-O—, or —NH—C₂₋₁₀ alkylene-NH—; wherein the heteroalkylene group is optionally substituted with R²⁰;
R¹⁹ is hydrogen, halogen, C₁₋₆ alkyl, C₁₋₆ haloalkyl, —OH, —OCH₃, or —OCF₃;
R²⁰ is hydrogen, halogen, C₁₋₆ alkyl, C₁₋₆ haloalkyl, or —OH;
R²¹ is hydrogen or C₁₋₆ alkyl;
R²² is hydrogen or C₁₋₆ alkyl, wherein the C₁₋₆ alkyl is unsubstituted or substituted with R²⁴;
R²³ is hydrogen, C₁₋₆ alkyl, or benzyl, wherein the C₁₋₆ alkyl or benzyl is unsubstituted or substituted with one, two, or three R²³ groups;
R²⁴ is —OH, —O(C₁₋₆ alkyl), —NH₂, —NH(C₁₋₆ alkyl), —N(C₁₋₆ alkyl)₂, —SH, or —S(C₁₋₆ alkyl);
R²⁵ is —OH, —O(C₁₋₆ alkyl), —NH₂, —NH(C₁₋₆ alkyl), —N(C₁₋₆ alkyl)₂, —NHS(O)₂(C₁₋₆ alkyl), C₁₋₆ alkyl, C₁₋₆ aminoalkyl, or OCH₂CH₂NHC(O)(C₁₋₆ aminoalkyl);
each Y¹, Y², Y³, and Y⁴ is independently —CH, —CF, or N;
Y⁵ is CH₂, NH, or O;
q is 0, 1, 2, 3, 4, or 5; and
r is 0, 1, 2, 3, 4, or 5.

In some embodiments, provided herein is a small-molecule drug conjugate having the structure of Formula (X-A) or Formula (X-B):

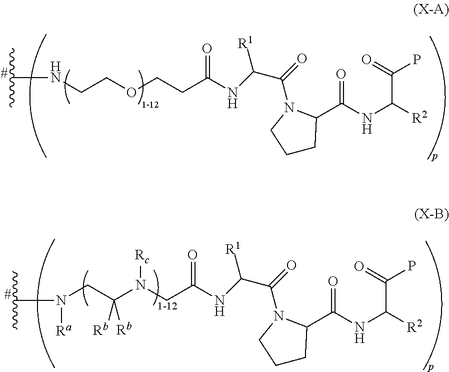

or a pharmaceutically acceptable salt thereof, wherein:
# denotes a bond to A¹, A², or T;
P is a therapeutic payload;
R¹ is hydrogen, —CH₂CONH₂, or —CH₂COOH;
R² is —CH₃, —CH(CH₃)₂, or —CH(CH₃)(CH₂—CH₃);
R^{a} is hydrogen or —CH₃;
R^{b} is in each instance, independently, hydrogen or CH₃; or two R^{b}, together with the carbon to which they are attached, form a carbonyl; and
R^{c} is hydrogen or —CH₃.

In some embodiments, provided herein is a compound or a pharmaceutically acceptable salt thereof, having a structure of Formula (II), Formula (III), Formula (IV), Formula (V), or Formula (VII):

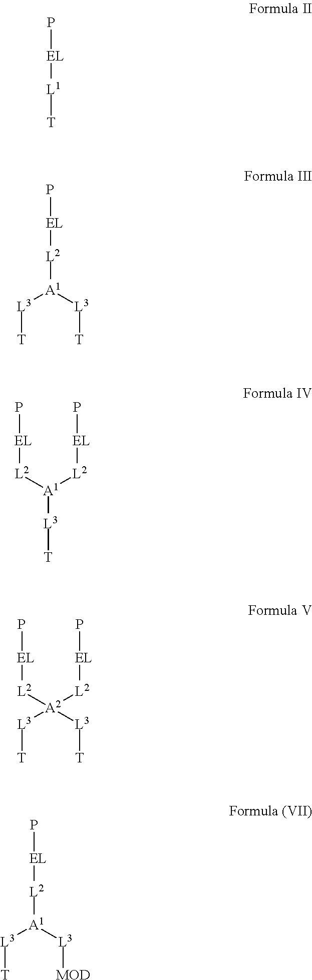

wherein:
P is a payload;
EL is a cleavable peptide linker;
each L¹, L², and L³ is independently a bivalent linker,
A¹ is a trivalent linker;
A² is a tetravalent linker;
T is a target protein binder; and
MOD is a pharmacokinetic modulating group.

In some embodiments, P is a tubulin polymerization inhibitor, topoisomerase inhibitor, oxidative phosphorylation inhibitor, kinase inhibitor, dihydrofolate reductase inhibitor, histone deacetylase inhibitor, microtubule inhibitor, or an immuonomodulator.

In some embodiments, provided herein is a compound or a pharmaceutically acceptable salt thereof, having a structure of Formula (II), Formula (III), Formula (IV), Formula (V), or Formula (VII), wherein:
each P is a tubulin polymerization inhibitor, a kinesin spindle protein inhibitor, a cyclin-dependent kinase inhibitor, an immunomodulator, an epidermal growth factor receptor inhibitor, or a microtubule inhibitor,
each EL is a cleavable peptide linker;
each L¹, L², and L³, is independently a bivalent linker,
A¹ is a trivalent linker;
A² is a tetravalent linker;
each T is a target protein binder (e.g., prostate-specific membrane antigen binder, carbonic anhydrase 9 binder, fibroblast activation protein binder, folate receptor binder, heat-shock protein 90 binder, an αvβ6 integrin binder or an αvβ3 integrin binder); and
MOD is a pharmacokinetic modulating group.

In some embodiments, P is an auristatin or auristatin derivative, a kinesin spindle protein inhibitor, a cyclin-dependent kinase 9 inhibitor, a toll-like receptor agonist, an epidermal growth factor receptor inhibitor, or a taxane.

In some embodiments, provided herein is a compound or a pharmaceutically acceptable salt thereof, having a structure of Formula (II), Formula (III), Formula (IV), Formula (V), or Formula (VII), wherein:
each T is a prostate-specific membrane antigen binder, carbonic anhydrase 9 binder, fibroblast activation protein binder, folate receptor binder, heat-shock protein 90 binder, or an αvβ6 integrin binder;
each L¹, L², and L³, is independently a bivalent linker;
A¹ is a trivalent linker;
A² is a tetravalent linker;
each EL is a cleavable peptide linker; and
each P is a payload (e.g., a tubulin polymerization inhibitor, topoisomerase inhibitor, oxidative phosphorylation inhibitor, kinase inhibitor, dihydrofolate reductase inhibitor, histone deacetylase inhibitor, microtubule inhibitor, or an immuonomodulator).

In some embodiments, each P is a camptothecin or camptothecin derivative, an auristatin or auristatin derivative, a kinesin spindle protein inhibitor, a cyclin-dependent kinase 9 inhibitor, a toll-like receptor agonist, an epidermal growth factor receptor inhibitor, or a taxane.

In some embodiments, provided herein is a compound having the structure of Formula II:

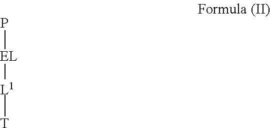

wherein:
P is a payload;
EL is a peptide linker;
L¹ is a bivalent linker, and
T is a target protein binder.

In some embodiments, the payload (P) of Formula II is a cytotoxic drug. In some embodiments, provided herein is a compound of Formula II, wherein the payload (P) is a topoisomerase inhibitor (Topo-i). In some embodiments, provided herein is a compound of Formula II, wherein the payload (P) is camptothecin, exatecan, deruxtecan, or a derivative thereof (e.g., 7-ethyl camptothecin, exatecan, N-alkyl exatecan, N-acyl exatecan, 5,6-methylenedioxoy camptothecin (“FL118”) or the like). In some embodiments, the payload (P) is 7-ethyl camptothecin. In some embodiments, the payload of Formula (II) is exatecan or FL118. In some embodiments, provided herein is a compound of Formula II, wherein the payload (P) is a kinesin spindle protein inhibitor (KSPi). In some embodiments, provided herein is a compound of Formula II, wherein the payload (P) is an auristatin. In some embodiments, the payload (P) of Formula II is an auristatin selected from auristatin E and monomethyl auristatin E (MMAE). In some embodiments, the payload of Formula II is monomethyl auristatin E (MMAE). In some embodiments, the payload of Formula II is an inhibitor of cyclin-dependent kinase 9 (CDK9) and/or positive transcription elongation factor (P-TEFb). In some embodiments, the payload of Formula II is an inhibitor of epidermal growth factor receptor (EGFR). In some embodiments, the payload of Formula II is a tubulin inhibitor (e.g., a tubulin polymerization inhibitor such as auristatin E (or monomethyl auristatin E), or a tubulin depolymerization inhibitor (e.g., paclitaxel)) In some embodiments, the payload of Formula II is an auristatin. In some embodiments, the payload of Formula II is monomethyl auristatin E. In some embodiments, the payload of Formula II is a taxane. In some embodiments, the payload of Formula II is paclitaxel. In some embodiments, the payload of Formula II is triptolide. In some embodiments, the payload of Formula II is erlotinib.

In some embodiments, the payload (P) of Formula II is an immune agonist. In some embodiments, the immune agonist of Formula II is a toll-like receptor agonist. In some embodiments, the payload (P) of Formula (II) is a toll-like receptor 7 and/or 8 (“TLR7/8) agonist. In some embodiments, the payload (P) of Formula H is resiquimod.
or a stereoisomer thereof; or a pharmaceutically acceptable salt thereof.

In some embodiments, provided herein is a compound having the structure of Formula III:

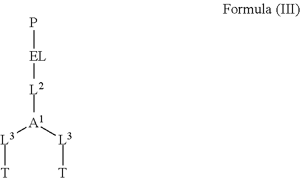

wherein:
P is a payload;
EL is a peptide linker;
each L² and L³ is independently a bivalent linker,
A¹ is a trivalent linker;
each T is a target protein binder.

In some embodiments, the payload (P) of Formula III is a cytotoxic drug or immune agonist as described herein. In some embodiments, provided herein is a compound of Formula III, wherein the payload (P) is a topoisomerase inhibitor (Topo-i). In some embodiments, provided herein is a compound of Formula III, wherein the payload (P) is a camptothecin, exatecan, deruxtecan, or derivative thereof (e.g., 7-ethyl camptothecin, exatecan, N-alkyl exatecan, N-acyl exatecan, 5,6-methylenedioxoy camptothecin (“FL118”) or the like. In some embodiments, provided herein is a compound of Formula III, wherein the payload (P) is a kinesin spindle protein inhibitor (KSPi). In some embodiments, provided herein is a compound of Formula III, wherein the payload (P) is an auristatin or an auristatin derivative (e.g., an auristatin selected from auristatin E, auristatin F and monomethyl auristatin E (MMAE) and monomethyl auristatin F (MMAF)). In some embodiments, the payload of Formula III is an inhibitor of cyclin-dependent kinase 9 (CDK9) and/or positive transcription elongation factor (P-TEFb). In some embodiments, the payload (P) of Formula (III) is a toll-like receptor 7 and/or 8 (“TLR7/8) agonist. In some embodiments, the payload (P) of Formula (III) is resiquimod. In some embodiments, the payload (P) is an EGFR inhibitor. In some embodiments, the payload (P) of Formula (III) is erlotinib. In some embodiments, the payload (P) of Formula (III) is an auristatin. In some embodiments, the payload (P) of Formula (III) is auristatin F or monomethyl auristatin E (MMAE).

In some embodiments, the target protein binder (T) is an integrin (e.g., alpha-v-beta-3 integrin) binder, alpha-v-beta-6 integrin) binder, PSMA binder, CAIX binder, FAP binder, folate receptor (FR) binder, Hsp90 binder, somatostatin binder, GLUT1 binder, APN binder, LRP1 binder, bombesin binder, GnRH binder, LHRH binder, MT1-MMP binder, P32 binder, phosphatidyl serine binder or sortilin binder. In some embodiments, the target protein binder (T) is a PSMA binder, a small molecule αᵥβ₃ integrin binder, a CAIX (CA9) binder, a FAP binder, a folate binder, or a Hsp90 binder. In some embodiments, the target protein binder (T) is a small molecule αᵥβ₃ (alpha-v-beta-3) integrin binder. In some embodiments, the target protein binder (T) is a FAP binder. In some embodiments, the target protein binder (T) is a PSMA binder. In some embodiments, the target protein binder (T) is a CAIX (CA9) binder. In some embodiments, the target protein binder (T) is a folate receptor (FR) binder. In some embodiments, the target protein binder (T) is a Hsp90 binder. In some embodiments, the target protein binder (T) is a PSMA binder, CAIX (CA9) binder, a FAP binder, a folate binder, or a Hsp90 binder.

In some embodiments, provided herein is a compound having the structure of Formula IV:

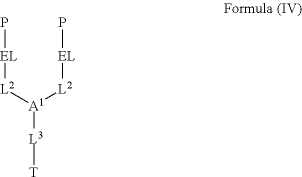

wherein:
each P is a payload;
each EL is a peptide linker;
each L² and L³ is independently a bivalent linker,
A¹ is a trivalent linker;
T is a target protein binder.

In some embodiments, provided herein is a compound having the structure of Formula V:

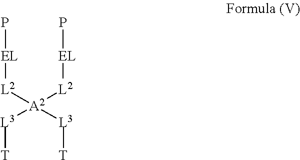

wherein:
each P is a payload;
each EL is a peptide linker;
each L² and L³ is independently a bivalent linker,
A² is a tetravalent linker;
each T is a target protein binder.

In some embodiments, provided herein is a compound having the structure of Formula VII:

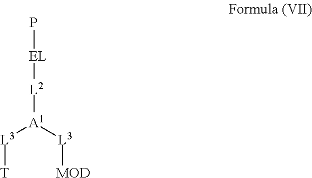

wherein:
P is a payload;
EL is a peptide linker;
each L² and L³ is independently a bivalent linker,
A¹ is a trivalent linker;
T is a target protein binder; and
MOD is a pharmacokinetic modulating group.

In some embodiments, provided herein is a compound or a pharmaceutically acceptable salt thereof, having a structure of Formula XI:

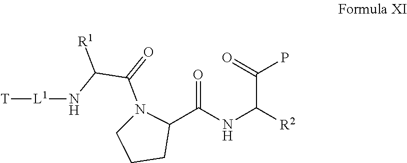

or a stereoisomer thereof; or a pharmaceutically acceptable salt thereof, wherein:
T is a target protein binder;
L¹ is a bivalent linker (e.g., a non-cleavable linker);
R¹ is hydrogen, C₁₋₆ alkyl, —CH₂CONH₂, —CH₂COOH, or —CH₂COOR³;
R² is —CH₃, —CH(CH₃)₂, —CH₂CH(CH₃)₂, or —CH(CH₃)(CH₂CH₃);
R³ is C₁₋₁₂ alkyl substituted with 0-3 instances of R⁴;
R⁴ is hydrogen, halogen, —C(O)OR⁵, —C(O)N(R⁵)₂, —N(R₅)₂, —N(R⁵)₃⁺, —OR⁵, —SR⁵, —S(O)R⁵, —S(O)₂R⁵, —S(O)₂N(R₅), —S(O)₂N(R₅)C(O)R₅, —S(O)₂N(R⁵)C(O)OR⁵, —N(R⁵)S(O)₂N(R⁵)₂, —N(R⁵)S(O)₂N(R⁵)C(O)R⁵, or —N(R⁵)S(O)₂N(R⁵)C(O)OR⁵;
R⁵ is in each instance, independently, hydrogen or C₁₋₆ alkyl; and
P is a payload (e.g., a therapeutic payload).

In some embodiments, L¹ is a bivalent linker comprising 2 to 20 polyethylene glycol groups.

In some embodiments, the compound of Formula XI has the structure:

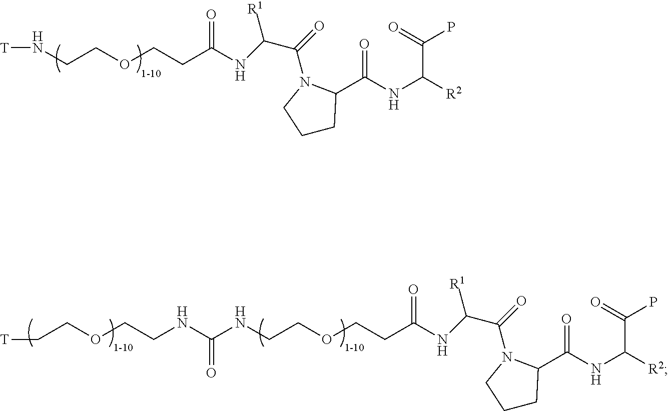

wherein:
T is a prostate-specific membrane antigen binder, carbonic anhydrase 9 binder, fibroblast activation protein binder, folate receptor binder, heat-shock protein 90 binder, or an αvβ6 integrin binder;
R¹ is hydrogen, —CH₃, —CH₂CH₃, —CH₂CH₂CH₃, —CH₂C(O)NH₂, or —CH₂C(O)OH;
R² is —CH₃, —CH(CH₃)₂, —CH₂CH(CH₃)₂, or —CH(CH₃)CH₂CH₃; and
P is a payload.

In some embodiments,
T is an αvβ3 integrin binder, and
P is a tubulin polymerization inhibitor, topoisomerase inhibitor, oxidative phosphorylation inhibitor, kinase inhibitor, dihydrofolate reductase inhibitor, histone deacetylase inhibitor, microtubule inhibitor, or an immuonomodulator.

In some embodiments,
T is a prostate-specific membrane antigen binder, carbonic anhydrase 9 binder, fibroblast activation protein binder, folate receptor binder, heat-shock protein 90 binder, an αvβ6 integrin binder, or an αvβ3 integrin binder, and
P is an auristatin or auristatin derivative, a kinesin spindle protein inhibitor, a cyclin-dependent kinase 9 inhibitor, a toll-like receptor agonist, an epidermal growth factor receptor inhibitor, or a taxane.

In some embodiments,
T is a prostate-specific membrane antigen binder, carbonic anhydrase 9 binder, fibroblast activation protein binder, folate receptor binder, heat-shock protein 90 binder, or an αvβ6 integrin binder, and
P is a camptothecin or camptothecin derivative, an auristatin or auristatin derivative, a kinesin spindle protein inhibitor, a cyclin-dependent kinase 9 inhibitor, a toll-like receptor agonist, an epidermal growth factor receptor inhibitor, or a taxane.

In some embodiments, P is a camptothecin or camptothecin derivative. In some embodiments, P is an auristatin or auristatin derivative.

In some embodiments, provided herein is a compound or a pharmaceutically acceptable salt thereof, having a structure of Formula XII:

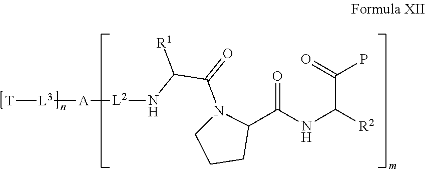

or a stereoisomer thereof; or a pharmaceutically acceptable salt thereof; wherein:
T is a target protein binder;
L² and L³ are each a bivalent linker (e.g., non-cleavable linker);
A is represents A¹ or A²,
   wherein A¹ is a trivalent linker, and A² is a tetravalent linker;
R¹ is hydrogen, C₁₋₆ alkyl, —CH₂CONH₂, —CH₂COOH, or —CH₂COOR³;
R² is —CH₃, —CH(CH₃)₂, —CH₂CH(CH₃)₂, or —CH(CH₃)(CH₂CH₃);
R³ is C₁₋₁₂ alkyl substituted with 0-3 instances of R⁴;
R⁴ is hydrogen, halogen, —C(O)OR⁵, —C(O)N(R⁵)₂, —N(R⁵)₂, —N(R⁵)₃⁺, —OR⁵, —SR⁵, —S(O)R⁵, —S(O)₂R⁵, —S(O)₂N(R⁵), —S(O)₂N(R⁵)C(O)R⁵, —S(O)₂N(R⁵)C(O)OR⁵, —N(R⁵)S(O)₂N(R⁵)₂, —N(R⁵)S(O)₂N(R⁵)C(O)R⁵, or —N(R⁵)S(O)₂N(R⁵)C(O)OR⁵;
R⁵ is in each instance, independently, hydrogen or C₁₋₆ alkyl;
P is a payload (e.g., a therapeutic payload);
m is 1 or 2; and
n is 1 or 2; wherein if n is 2, one instance of T is optionally replaced with MOD;
   wherein MOD is a pharmacokinetic modulating group (e.g., COOH).

In some embodiments, each of L² and L³ is a bivalent linker comprising 2 to 20 polyethylene glycol groups. In some embodiments, the compound of Formula XII has the structure:

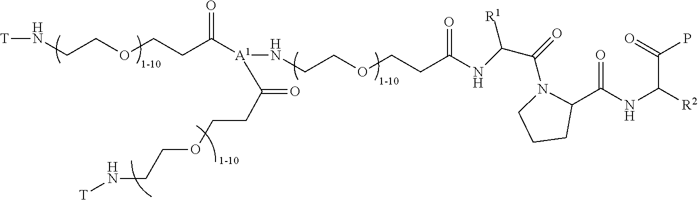

wherein:
each T is a target protein binder (e.g., a prostate-specific membrane antigen binder, carbonic anhydrase 9 binder, fibroblast activation protein binder, folate receptor binder, heat-shock protein 90 binder, or αvβ6 integrin binder);
R¹ is hydrogen, —CH₃, —CH₂CH₃, —CH₂CH₂CH₃, —CH₂C(O)NH₂, or —CH₂C(O)OH;
R² is —CH₃, —CH(CH₃)₂, —CH₂CH(CH₃)₂, or —CH(CH₃)CH₂CH₃; and
P is a payload.

In some embodiments,
each T is an αvβ3 integrin binder, and
P is a tubulin polymerization inhibitor, topoisomerase inhibitor, oxidative phosphorylation inhibitor, kinase inhibitor, dihydrofolate reductase inhibitor, histone deacetylase inhibitor, microtubule inhibitor, or an immuonomodulator.

In some embodiments,
each T is a prostate-specific membrane antigen binder, carbonic anhydrase 9 binder, fibroblast activation protein binder, folate receptor binder, heat-shock protein 90 binder, an αvβ6 integrin binder, or an αvβ3 integrin binder, and
P is an auristatin or auristatin derivative, a kinesin spindle protein inhibitor, a cyclin-dependent kinase 9 inhibitor, a toll-like receptor agonist, an epidermal growth factor receptor inhibitor, or a taxane.

In some embodiments,
each T is a prostate-specific membrane antigen binder, carbonic anhydrase 9 binder, fibroblast activation protein binder, folate receptor binder, heat-shock protein 90 binder, or an αvβ6 integrin binder, and
P is a camptothecin or camptothecin derivative, an auristatin or auristatin derivative, a kinesin spindle protein inhibitor, a cyclin-dependent kinase 9 inhibitor, a toll-like receptor agonist, an epidermal growth factor receptor inhibitor, or a taxane.

In some embodiments, P is a camptothecin or camptothecin derivative. In some embodiments, P is an auristatin or auristatin derivative.

In some embodiments, provided herein is a compound or a pharmaceutically acceptable salt thereof, having the structure of Formula XI-KSP or XII-KSP:

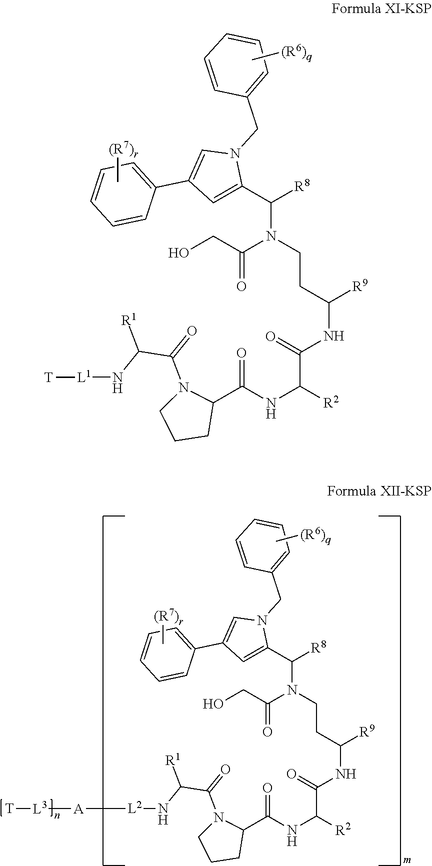

or a stereoisomer thereof; or a pharmaceutically acceptable salt thereof, wherein:
T is a target protein binder;
L¹, L², and L³ are each a bivalent linker (e.g., non-cleavable linker);
A is represents A¹ or A²,
   wherein A¹ is a trivalent linker, and A² is a tetravalent linker;
R¹ is hydrogen, C₁₋₆ alkyl, —CH₂CONH₂, —CH₂COOH, or —CH₂COOR³;
R² is —CH₃, —CH(CH₃)₂, —CH₂CH(CH₃)₂, or —CH(CH₃)(CH₂CH₃);
R³ is C₁₋₁₂ alkyl substituted with 0-3 instances of R⁴;
R⁴ is hydrogen, halogen, —C(O)OR, —C(O)N(RV)₂, —N(R⁵)₂, —N(R⁵)₃⁺, —OR, —SR⁵, —S(O)R⁵, —S(O)₂R⁵, —S(O)₂N(R⁵), —S(O)₂N(R⁵)C(O)R⁵, —S(O)₂N(R⁵)C(O)OR⁵, —N(R⁵)S(O)₂N(R⁵)₂, —N(R⁵)S(O)₂N(R⁵)C(O)R⁵, or —N(R⁵)S(O)₂N(R⁵)C(O)OR⁵;
R⁵ is in each instance, independently, hydrogen or C₁₋₆ alkyl;
each R⁶ and R⁵ is independently hydrogen, halogen, CN, —C₁₋₆ alkyl, or C₁₋₆ haloalkyl;
R⁸ is hydrogen, halogen, CN, —C₁₋₆ alkyl, C₁₋₆ haloalkyl, C₃₋₆ cycloalkyl, or 5- to 7-membered heterocycloalkyl;
R⁹ is hydrogen, halogen, CN, C₁₋₆ alkyl, —C(O)NH₂, —C(O)NHC₁₋₆ alkyl, —C(O)N(C₁₋₆ alkyl)₂, —C(O)NH—C₁₋₆ alkyl-C(O)NH—C₁₋₆ alkyl, —C(O)NHC₁₋₆ alkyl-NHC(O)—C₁₋₆ alkyl, —NH₂, —NHC₁₋₆ alkyl, —N(C₁₋₆ alkyl)₂, —NHC(O)C₁₋₆ alkyl, —OH, or —OC₁₋₆ alkyl; wherein each C₁₋₆ alkyl is substituted with 0-5 R¹⁰;
R¹⁰ is in each instance independently selected from the group consisting of hydrogen, halogen, CN, —COOH, —CONH₂, —NH₂, —NHCH₃, —N(CH₃)₂, —OH, and —OCH₃;
m is 1 or 2;
n is 1 or 2; wherein if n is 2, one instance of T is optionally replaced with MOD;
   wherein MOD is a pharmacokinetic modulating group (e.g., COOH);
q is 0, 1, 2, 3, 4, or 5; and
r is 0, 1, 2, 3, 4, or 5.

In some embodiments, each T is prostate-specific membrane antigen binder, carbonic anhydrase 9 binder, fibroblast activation protein binder, folate receptor binder, heat-shock protein 90 binder, an αvβ6 integrin binder, or an αvβ3 integrin binder.

In some embodiments, provided herein is a compound or a pharmaceutically acceptable salt thereof, having the structure of Formula XI-KSP′ or XII-KSP′:

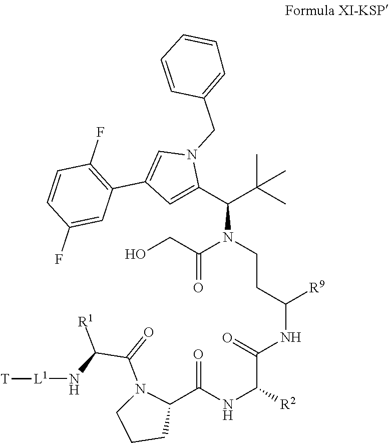

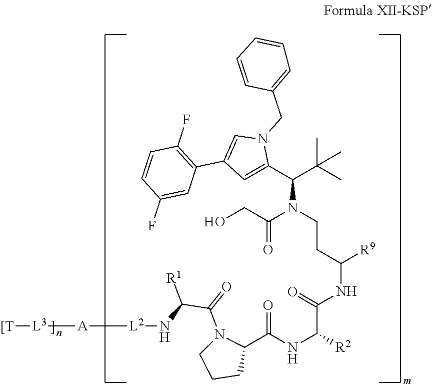

or a stereoisomer thereof; or a pharmaceutically acceptable salt thereof, wherein:
T is a target protein binder;
L¹, L², and L³ are each a bivalent linker (e.g., non-cleavable linker);
A is represents A¹ or A²,
   wherein A¹ is a trivalent linker, and A² is a tetravalent linker;
R¹ is hydrogen, —CH₂CONH₂, or —CH₂COOH;
R² is —CH₃ or —CH(CH₃)₂.
R⁹ is hydrogen, halogen, CN, C₁₋₆ alkyl, —C(O)NH₂, —C(O)NHC₁₋₆ alkyl, —C(O)N(C₁₋₆ alkyl)₂, —C(O)NH—C₁₋₆ alkyl-C(O)NH—C₁₋₆ alkyl, —C(O)NHC₁₋₆ alkyl-NHC(O)—C₁₋₆ alkyl, —NH₂, —NHC₁₋₆ alkyl, —N(C₁₋₆ alkyl)₂, —NHC(O)C₁₋₆ alkyl, —OH, or —OC₁₋₆ alkyl; wherein each C₁₋₆ alkyl is substituted with 0-5 R¹⁰;
R¹⁰ is in each instance independently selected from the group consisting of hydrogen, halogen, CN, —COOH, —CONH₂, —NH₂, —NHCH₃, —N(CH₃)₂, —OH, and —OCH₃;
m is 1 or 2; and
n is 1 or 2; wherein if n is 2, one instance of T is optionally replaced with MOD;
   wherein MOD is a pharmacokinetic modulating group (e.g., COOH).

In some embodiments, provided herein is a compound or a pharmaceutically acceptable salt thereof, having the structure of Formula XI-TOPO or XII-TOPO:

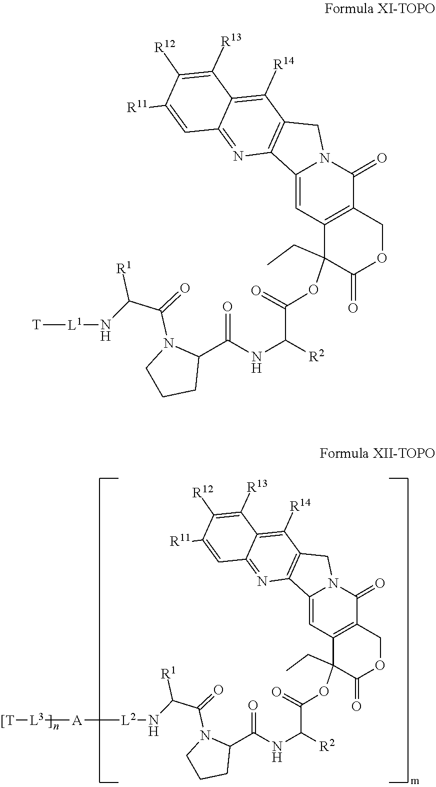

or a stereoisomer thereof; or a pharmaceutically acceptable salt thereof; wherein:
T is a target protein binder;
L¹, L², and L³ are each a bivalent linker (e.g., non-cleavable linker);
A is represents A¹ or A²,
   wherein A¹ is a trivalent linker, and A² is a tetravalent linker;
R¹ is hydrogen, C₁₋₆ alkyl, —CH₂CONH₂, —CH₂COOH, or —CH₂COOR³;
R² is —CH₃, —CH(CH₃)₂, —CH₂CH(CH₃)₂, or —CH(CH₃)(CH₂CH₃);
R³ is C₁₋₁₂ alkyl substituted with 0-3 instances of R⁴;
R⁴ is hydrogen, halogen, —C(O)OR⁵, —C(O)N(R⁵)₂, —N(R⁵)₂, —N(R⁵)₃⁺, —OR⁵, —SR⁵, —S(O)R⁵, —S(O)₂R⁵, —S(O)₂N(R⁵), —S(O)₂N(R⁵)C(O)R⁵, —S(O)₂N(R⁵)C(O)OR⁵, —N(R⁵)S(O)₂N(R⁵)₂, —N(R⁵)S(O)₂N(R⁵)C(O)R⁵, or —N(R⁵)S(O)₂N(R⁵)C(O)OR⁵;
R⁵ is in each instance, independently, hydrogen or C₁₋₆ alkyl;
R¹¹ and R¹² are each independently hydrogen, halogen, C₁₋₆ alkyl, C₁₋₆ haloalkyl, or —OH; or R¹¹ and R¹², taken together, form a 5- or 6-membered heterocycle;
R¹³ and R¹⁴ are each independently hydrogen, C₁₋₆ alkyl, or C₁₋₆ alkylamine; or R¹³ and R¹⁴, taken together, form a C₆ carbocycle substituted with —N(R¹⁵)₂;
each R¹⁵ is independently hydrogen, C₁₋₆ alkyl, —C(O)C₁₋₆ alkyl, —C(O)NHC₁₋₆ alkyl, or —C(O)OC₁₋₆ alkyl; wherein the C₁₋₆ alkyl of R¹⁵ is optionally substituted with halogen, hydroxy, phenyl, or heteroaryl; or R¹⁵ is a cleavable prodrug group;
m is 1 or 2; and
n is 1 or 2; wherein if n is 2, one instance of T is optionally replaced with MOD;
   wherein MOD is a pharmacokinetic modulating group (e.g., COOH).

In some embodiments, T is a prostate-specific membrane antigen binder, carbonic anhydrase 9 binder, fibroblast activation protein binder, folate receptor binder, heat-shock protein 90 binder, or an αvβ6 integrin binder.

In some embodiments, provided herein is a compound or a pharmaceutically acceptable salt thereof, having the structure of Formula XI-TOPO′ or XII-TOPO′:

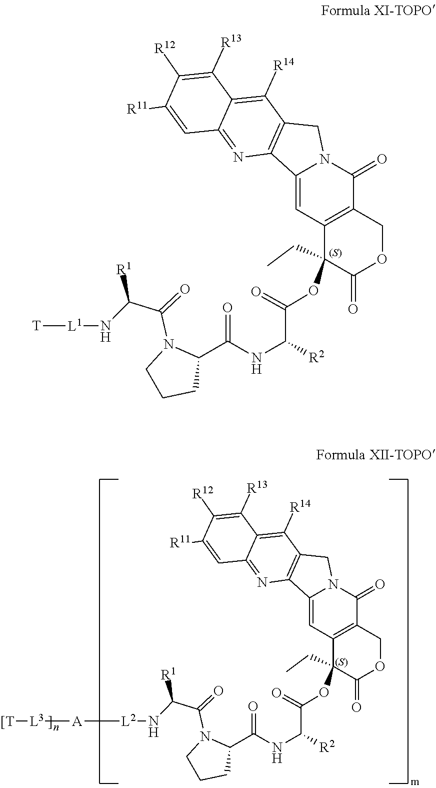

or a stereoisomer thereof; or a pharmaceutically acceptable salt thereof; wherein:
T is a target protein binder;
L¹, L², and L³ are each a bivalent linker (e.g., non-cleavable linker);
A is represents A¹ or A²,
   wherein A¹ is a trivalent linker, and A² is a tetravalent linker;
R¹ is hydrogen, —CH₂CONH₂, or —CH₂COOH;
R² is —CH₃ or —CH(CH₃)₂.
R¹¹ and R¹² are each independently hydrogen, halogen, C₁₋₆ alkyl, C₁₋₆ haloalkyl, or —OH; or R¹¹ and R¹², taken together, form a 5- or 6-membered heterocycle;
R¹³ and R¹⁴ are each independently hydrogen, C₁₋₆ alkyl, or C₁₋₆ alkylamine; or R¹³ and R¹⁴, taken together, form a C₆ carbocycle substituted with —N(R¹⁵)₂;
each R¹⁵ is independently hydrogen, C₁₋₆ alkyl, —C(O)C₁₋₆ alkyl, —C(O)NHC₁₋₆ alkyl, or —C(O)OC₁₋₆ alkyl; wherein the C₁₋₆ alkyl of R¹⁵ is optionally substituted with halogen, hydroxy, phenyl, or heteroaryl; or R¹⁵ is a cleavable prodrug group;
m is 1 or 2; and
n is 1 or 2; wherein if n is 2, one instance of T is optionally replaced with MOD;
   wherein MOD is a pharmacokinetic modulating group (e.g., COOH).

In some embodiments, provided herein is a compound or a pharmaceutically acceptable salt thereof, having the structure of Formula XI-CDK9 or XII-CDK9:

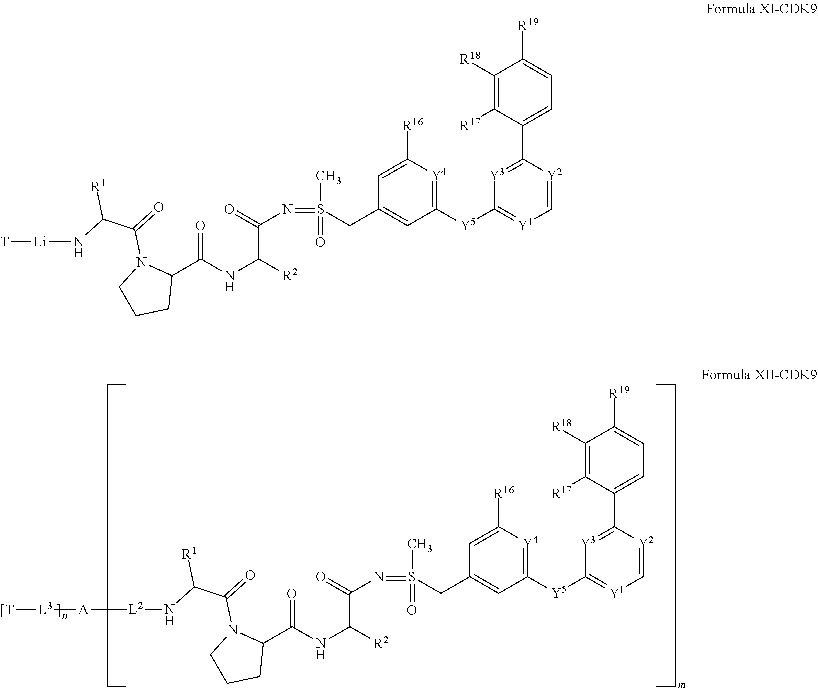

or a stereoisomer thereof; or a pharmaceutically acceptable salt thereof, wherein:
T is a target protein binder;
L¹, L², and L³ are each a bivalent linker (e.g., non-cleavable linker);
A is represents A¹ or A²,
   wherein A¹ is a trivalent linker, and A² is a tetravalent linker;
R¹ is hydrogen, C₁₋₆ alkyl, —CH₂CONH₂, —CH₂COOH, or —CH₂COOR³;
R² is —CH₃, —CH(CH₃)₂, —CH₂CH(CH₃)₂, or —CH(CH₃)(CH₂CH₃);
R³ is C₁₋₁₂ alkyl substituted with 0-3 instances of R⁴;
R⁴ is hydrogen, halogen, —C(O)OR⁵, —C(O)N(R⁵)₂, —N(R⁵)₂, —N(R⁵)₃⁺, —OR⁵, —SR⁵, —S(O)R⁵, —S(O)₂R⁵, —S(O)₂N(R⁵), —S(O)₂N(R⁵)C(O)R⁵, —S(O)₂N(R⁵)C(O)OR⁵, —N(R⁵)S(O)₂N(R⁵)₂, —N(R⁵)S(O)₂N(R⁵)C(O)R⁵, or —N(R⁵)S(O)₂N(R⁵)C(O)OR⁵;
R⁵ is in each instance, independently, hydrogen or C₁₋₆ alkyl;
R¹⁶ is hydrogen, halogen, C₁₋₆ alkyl, C₁₋₆ haloalkyl, —OH, —OCH₃, or —OCF₃;
R¹⁷ is hydrogen, halogen, C₁₋₆ alkyl, C₁₋₆ haloalkyl, —OH, —OCH₃, or —OCF₃;
R¹⁸ is hydrogen, halogen, C₁₋₆ alkyl, C₁₋₆ haloalkyl, —OH, —OCH₃, or —OCF₃;
or R¹⁶ and R¹⁷, taken together, form a heteroalkylene group of the formula: —O—C₂₋₁₀ alkylene-O—, —NH—C₂₋₁₀ alkylene-O—, or —NH—C₂₋₁₀ alkylene-NH—; or R⁶ and R^{1B}, taken together, form a heteroalkylene group of the formula: —O—C₂₋₁₀ alkylene-O—, —NH—C₂₋₁₀ alkylene-O—, or —NH—C₂₋₁₀ alkylene-NH—;
R¹⁹ is hydrogen, halogen, C₁₋₆ alkyl, C₁₋₆ haloalkyl, —OH, —OCH₃, or —OCF₃;
each Y¹, Y², Y³, and Y⁴ is independently —CH, —CF, or N;
Y⁵ is CH₂, NH, or O;
m is 1 or 2; and
n is 1 or 2; wherein if n is 2, one instance of T is optionally replaced with MOD;
   wherein MOD is a pharmacokinetic modulating group (e.g., COOH).

In some embodiments, T is a prostate-specific membrane antigen binder, carbonic anhydrase 9 binder, fibroblast activation protein binder, folate receptor binder, heat-shock protein 90 binder, or an αvβ6 integrin binder.

In some embodiments, provided herein is a compound or a pharmaceutically acceptable salt thereof, having the structure of Formula XI-CDK9′ or XII-CDK9′:

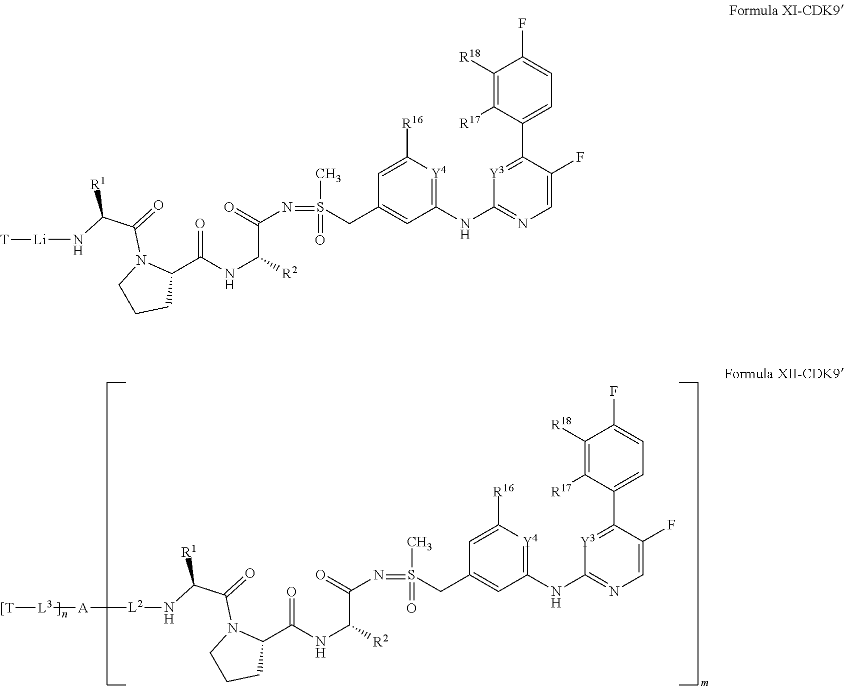

or a stereoisomer thereof; or a pharmaceutically acceptable salt thereof, wherein:
T is a target protein binder;
L¹, L², and L³ are each a bivalent linker (e.g., non-cleavable linker);
A is represents A¹ or A²,
   wherein A¹ is a trivalent linker, and A² is a tetravalent linker;
R¹ is hydrogen, —CH₂CONH₂, or —CH₂COOH;
R² is —CH₃ or —CH(CH₃)₂.
R¹⁶ is hydrogen,
R¹⁷ is hydrogen, —OH, —OCH₃, or —OCF₃;
R¹⁸ is hydrogen;
or R¹⁶ and R¹⁷, taken together, form a heteroalkylene group of the formula: —O—C2-10 alkylene-O—, —NH—C₂₋₁₀ alkylene-O—, or —NH—C₂₋₁₀ alkylene-NH—;
or R¹⁶ and R¹⁷, taken together, form a heteroalkylene group of the formula: —O—C2-10 alkylene-O—, —NH—C₂₋₁₀ alkylene-O—, or —NH—C₂₋₁₀ alkylene-NH—;
Y³ is CH or N;
Y⁴ is CH or N;
m is 1 or 2; and
n is 1 or 2; wherein if n is 2, one instance of T is optionally replaced with MOD;
   wherein MOD is a pharmacokinetic modulating group (e.g., COOH).

In some embodiments, provided herein is a compound or a pharmaceutically acceptable salt thereof, having the structure of Formula XI-TP or XII-TP:

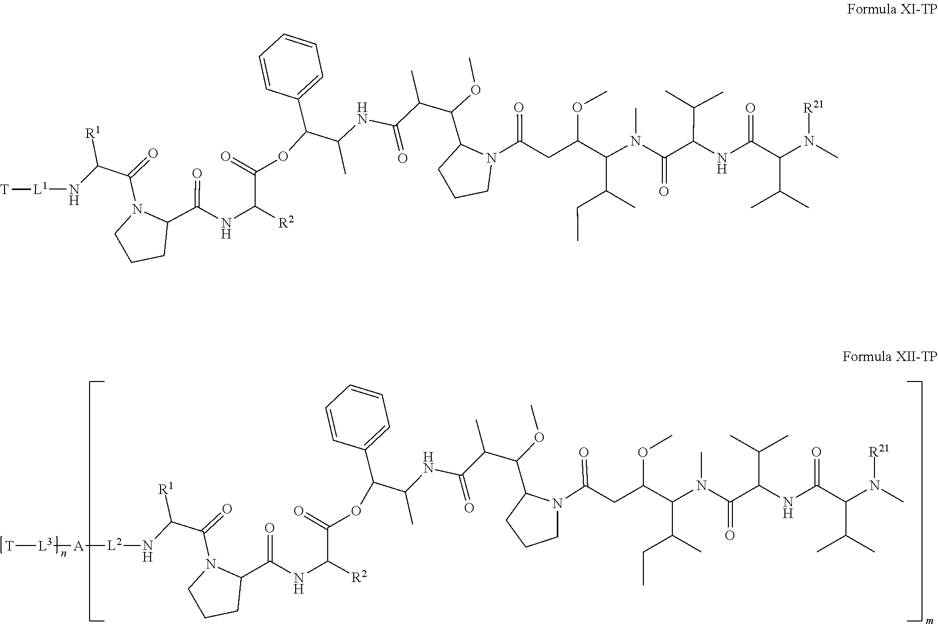

or a stereoisomer thereof; or a pharmaceutically acceptable salt thereof, wherein:
T is a target protein binder;
L¹, L², and L³ are each a bivalent linker (e.g., non-cleavable linker);
A is represents A¹ or A²,
   wherein A¹ is a trivalent linker, and A² is a tetravalent linker;
R¹ is hydrogen, C₁₋₆ alkyl, —CH₂CONH₂, —CH₂COOH, or —CH₂COOR³;
R² is —CH₃, —CH(CH₃)₂, —CH₂CH(CH₃)₂, or —CH(CH₃)(CH₂CH₃);
R³ is C₁₋₁₂ alkyl substituted with 0-3 instances of R⁴;
R⁴ is hydrogen, halogen, —C(O)OR⁵, —C(O)N(R⁵)₂, —N(R⁵)₂, —N(R⁵)₃⁺, —OR⁵, —SR⁵, —S(O)R⁵, —S(O)₂R⁵, —S(O)₂N(R⁵), —S(O)₂N(R⁵)C(O)R⁵, —S(O)₂N(R⁵)C(O)OR⁵, —N(R⁵)S(O)₂N(R⁵)₂, —N(R⁵)S(O)₂N(R⁵)C(O)R⁵, or —N(R⁵)S(O)₂N(R⁵)C(O)OR⁵;
R⁵ is in each instance, independently, hydrogen or C₁₋₆ alkyl;
R²¹ is hydrogen or —CH₃;
m is 1 or 2; and
n is 1 or 2; wherein if n is 2, one instance of T is optionally replaced with MOD;
   wherein MOD is a pharmacokinetic modulating group (e.g., COOH).

In some embodiments, T is a prostate-specific membrane antigen binder, carbonic anhydrase 9 binder, fibroblast activation protein binder, folate receptor binder, heat-shock protein 90 binder, or an αvβ6 integrin binder.

In some embodiments, provided herein is a compound or a pharmaceutically acceptable salt thereof, having the structure of Formula XI-TP′ or XII-TP′:

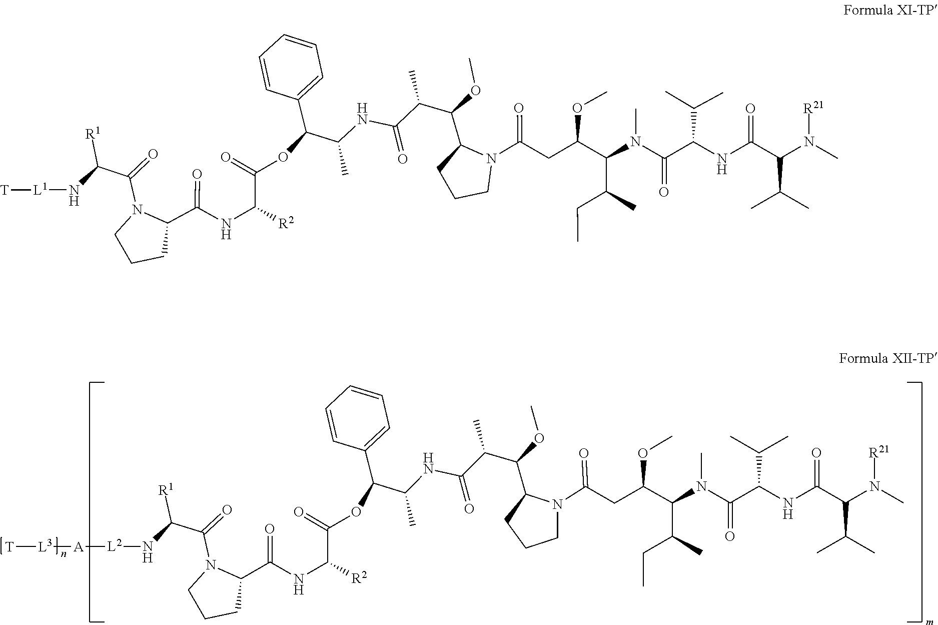

or a stereoisomer thereof; or a pharmaceutically acceptable salt thereof, wherein:
T is a target protein binder;
L¹, L², and L³ are each a bivalent linker (e.g., non-cleavable linker);
A is represents A¹ or A²,
   wherein A¹ is a trivalent linker, and A² is a tetravalent linker;
R¹ is hydrogen, —CH₂CONH₂, or —CH₂COOH;
R² is —CH₃ or —CH(CH₃)₂.
R²¹ is hydrogen or —CH₃;
m is 1 or 2; and
n is 1 or 2; wherein if n is 2, one instance of T is optionally replaced with MOD;
   wherein MOD is a pharmacokinetic modulating group (e.g., COOH).

In some embodiments, provided herein is a compound or a pharmaceutically acceptable salt thereof, having the structure of Formula XI-TP′ or XII-TP′:

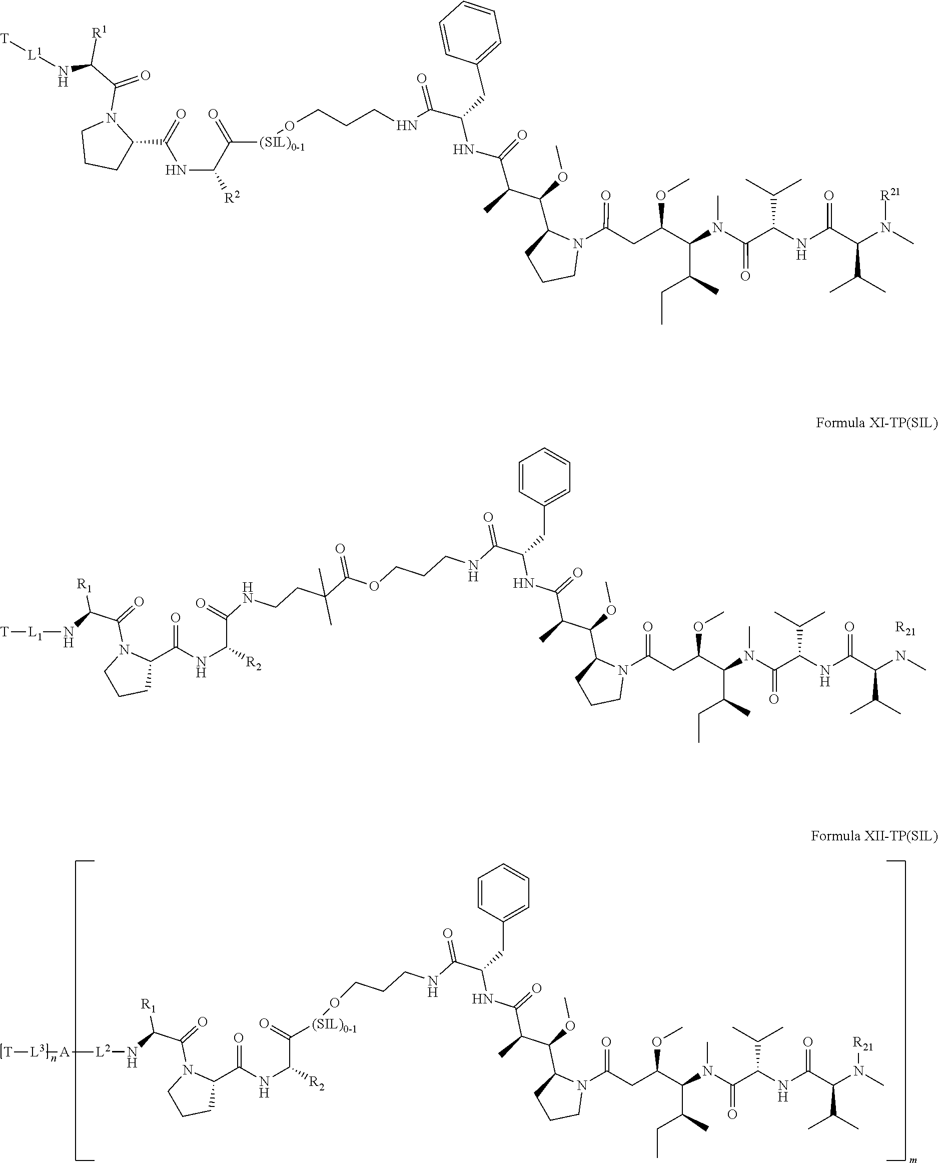

or a stereoisomer thereof; or a pharmaceutically acceptable salt thereof; wherein:
T is a target protein binder;
L¹, L², and L³ are each a bivalent linker (e.g., non-cleavable linker);
A is represents A¹ or A²,
   wherein A¹ is a trivalent linker, and A² is a tetravalent linker;
R¹ is hydrogen, —CH₂CONH₂, or —CH₂COOH;
R² is —CH₃ or —CH(CH₃)₂.
R²¹ is hydrogen or —CH₃;
SIL is a self-immolative linker (e.g., a C₂₋₁₈ heteroalkylene group)
m is 1 or 2; and
n is 1 or 2; wherein if n is 2, one instance of T is optionally replaced with MOD;
   wherein MOD is a pharmacokinetic modulating group (e.g., COOH).

In some embodiments, provided herein is a compound or a pharmaceutically acceptable salt thereof, having the structure of Formula XI-TLR or XII-TLR:

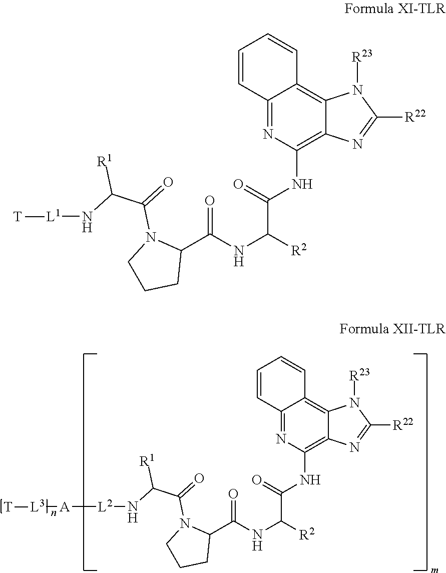

or a stereoisomer thereof; or a pharmaceutically acceptable salt thereof; wherein:
T is a target protein binder;
L¹, L², and L³ are each a bivalent linker (e.g., non-cleavable linker);
A is represents A¹ or A²,
   wherein A¹ is a trivalent linker, and A² is a tetravalent linker;
R¹ is hydrogen, C₁₋₆ alkyl, —CH₂CONH₂, —CH₂COOH, or —CH₂COOR³;
R² is —CH₃, —CH(CH₃)₂, —CH₂CH(CH₃)₂, or —CH(CH₃)(CH₂CH₃);
R³ is C₁₋₁₂ alkyl substituted with 0-3 instances of R⁴;
R⁴ is hydrogen, halogen, —C(O)OR⁵, —C(O)N(R⁵)₂, —N(R⁵)₂, —N(R⁵)₃⁺, —OR⁵, —SR⁵, —S(O)R⁵, —S(O)₂R⁵, —S(O)₂N(R⁵), —S(O)₂N(R⁵)C(O)R⁵, —S(O)₂N(R⁵)C(O)OR⁵, —N(R⁵)S(O)₂N(R⁵)₂, —N(R⁵)S(O)₂N(R⁵)C(O)R⁵, or —N(R⁵)S(O)₂N(R⁵)C(O)OR⁵;
R⁵ is in each instance, independently, hydrogen or C₁₋₆ alkyl;
R²² is hydrogen or C₁₋₆ alkyl, wherein the C₁₋₆ alkyl is unsubstituted or substituted with R²⁴.
R²³ is hydrogen, C₁₋₆ alkyl, or benzyl, wherein the C₁₋₆ alkyl or benzyl is unsubstituted or substituted with one, two, or three R²³ groups;
R²⁴ is —OH, —O(C₁₋₆ alkyl), —NH₂, —NH(C₁₋₆ alkyl), —N(C₁₋₆ alkyl)₂, —SH, or —S(C₁₋₆ alkyl);
R²⁵ is —OH, —O(C₁₋₆ alkyl), —NH₂, —NH(C₁₋₆ alkyl), —N(C₁₋₆ alkyl)₂, —NHS(O)₂(C₁₋₆ alkyl), C₁₋₆ alkyl, C₁₋₆ aminoalkyl, or OCH₂CH₂NHC(O)(C₁₋₆ aminoalkyl);
m is 1 or 2; and
n is 1 or 2; wherein if n is 2, one instance of T is optionally replaced with MOD;
   wherein MOD is a pharmacokinetic modulating group (e.g., COOH).

In some embodiments, T is a prostate-specific membrane antigen binder, carbonic anhydrase 9 binder, fibroblast activation protein binder, folate receptor binder, heat-shock protein 90 binder, or an αvβ6 integrin binder.

In some embodiments, provided herein is a compound or a pharmaceutically acceptable salt thereof, having the structure of Formula XI-TLR′ or XII-TLR′:

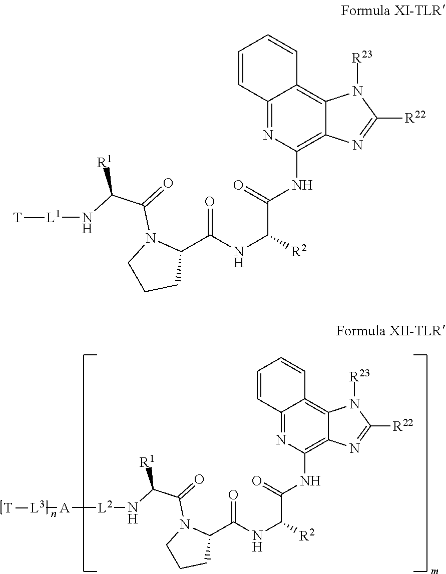

or a stereoisomer thereof; or a pharmaceutically acceptable salt thereof, wherein:
T is a target protein binder;
L¹, L², and L³ are each a bivalent linker (e.g., non-cleavable linker);
A is represents A¹ or A²,
   wherein A¹ is a trivalent linker, and A² is a tetravalent linker;
R¹ is hydrogen, —CH₂CONH₂, or —CH₂COOH;
R² is —CH₃ or —CH(CH₃)₂.
R²² is hydrogen or C₁₋₆ alkyl, wherein the C₁₋₆ alkyl is unsubstituted or substituted with R²⁴;
R²³ is hydrogen, C₁₋₆ alkyl, or benzyl, wherein the C₁₋₆ alkyl or benzyl is unsubstituted or substituted with one, two, or three R²³ groups;
R²⁴ is —OH, —O(C₁₋₆ alkyl), —NH₂, —NH(C₁₋₆ alkyl), —N(C₁₋₆ alkyl)₂, —SH, or —S(C₁₋₆ alkyl);
R²⁵ is —OH, —O(C₁₋₆ alkyl), —NH₂, —NH(C₁₋₆ alkyl), —N(C₁₋₆ alkyl)₂, —NHS(O)₂(C₁₋₆ alkyl), C₁₋₆ alkyl, C₁₋₆ aminoalkyl, or OCH₂CH₂NHC(O)(C₁₋₆ aminoalkyl);
m is 1 or 2; and
n is 1 or 2; wherein if n is 2, one instance of T is optionally replaced with MOD;
   wherein MOD is a pharmacokinetic modulating group (e.g., COOH).

In some embodiments, the target protein binder (T) is an αᵥβ₃ integrin (e.g., alpha-v-beta-3 integrin) binder, an αᵥβ₆ (avb6) integrin binder, PSMA binder, CAIX binder, FAP binder, folate receptor (FR) binder, Hsp90 binder, somatostatin binder, GLUT1 binder, APN binder, LRP1 binder, bombesin binder, GnRH binder, LHRH binder, MT1-MMP binder, P32 binder, phosphatidyl serine binder or sortilin binder. In some embodiments, the target protein binder (T) is a PSMA binder, a small molecule αᵥβ₃ integrin binder, a CAIX (CA9) binder, a FAP binder, a folate binder, or a Hsp90 binder. In some embodiments, the target protein binder (T) is a small molecule αᵥβ₃ (alpha-v-beta-3) integrin binder. In some embodiments, the target protein binder (T) is a small molecule an αᵥβ₆ (avb6) integrin binder. In some embodiments, the target protein binder (T) is a FAP binder. In some embodiments, the target protein binder (T) is a PSMA binder. In some embodiments, the target protein binder (T) is a CAIX (CA9) binder. In some embodiments, the target protein binder (T) is a folate receptor (FR) binder. In some embodiments, the target protein binder (T) is a Hsp90 binder. In some embodiments, the target protein binder (T) is a PSMA binder, CAIX (CA9) binder, a FAP binder, a folate binder, or a Hsp90 binder.

In some embodiments, T is:

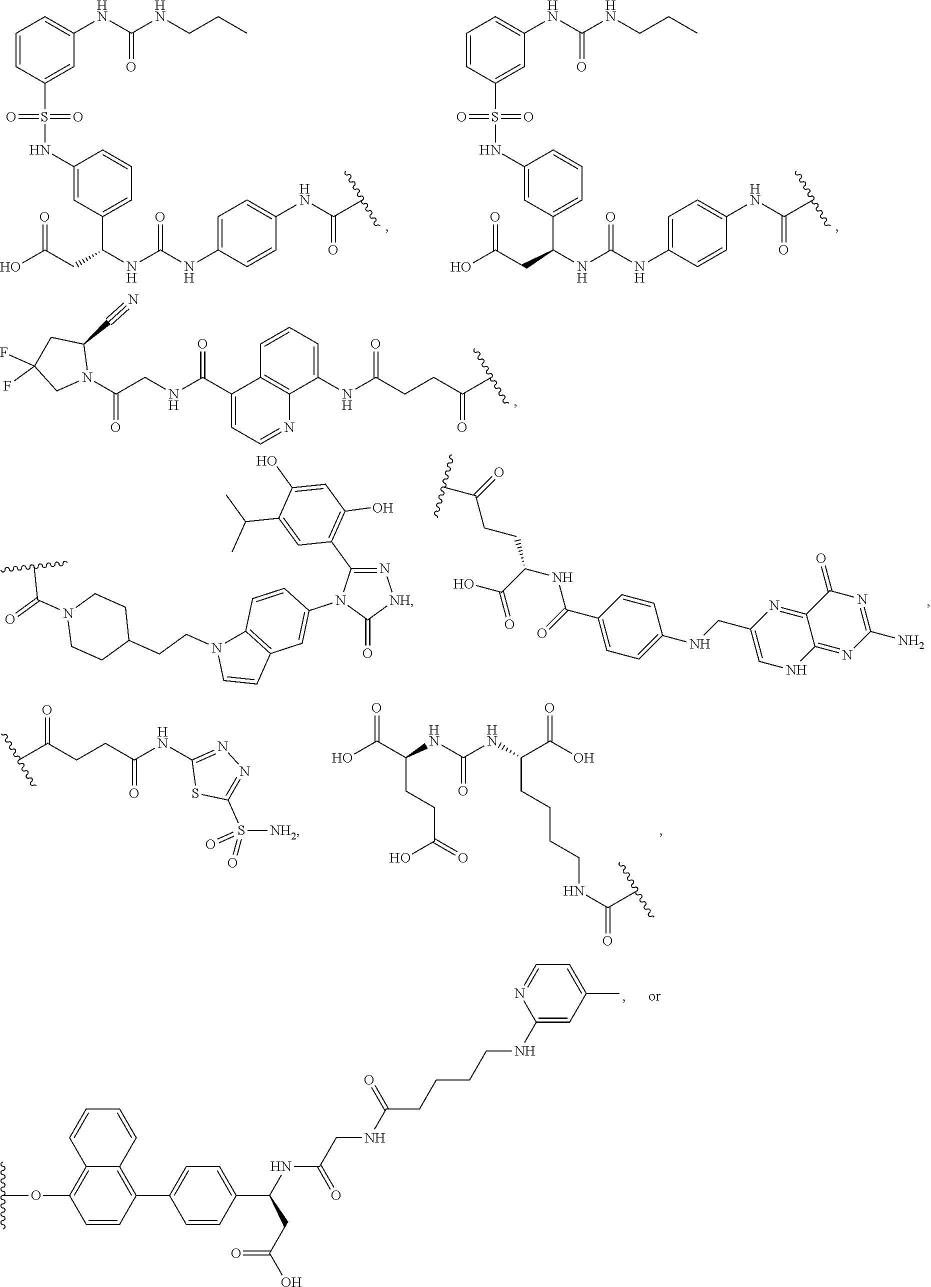

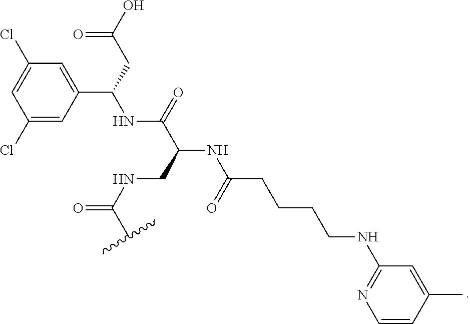 [SMILES: *C(=O)NC[C@H](NC(=O)CCCCNc1cc(C)ccn1)C(=O)N[C@@H](CC(=O)O)c1cc(Cl)cc(Cl)c1]

In some embodiments, each T is:

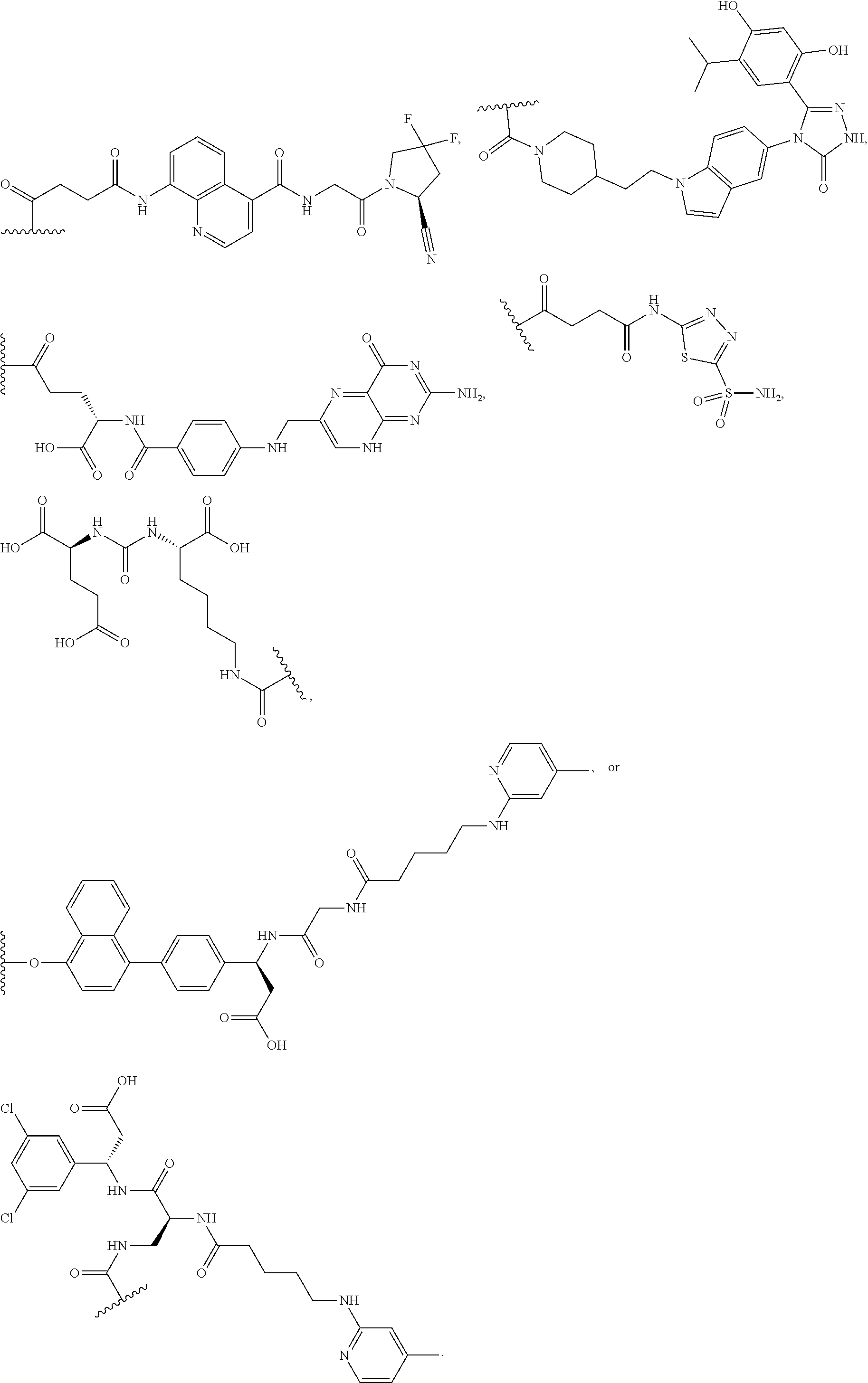

In some embodiments, a linker (e.g., one or more of L¹, L2, or L³) is an optionally substituted polyamine or polyamide linker. In some embodiments, the polyamine or polyamide linker of L¹, L², and/or L³ is substituted with one or more carbonyl groups. In some embodiments, the polyamine or polyamide linker of L¹, L², and/or L³ forms an aminium ion (or optionally multiple aminium ions) in an acidic tumor microenvironment. In some embodiments, the polyamine or polyamide linker of L¹, L², and/or L³ is selectively retained in a tumor microenvironment. In some embodiments, L¹, L², and/or L³ comprises a polyethylene glycol (PEG) linker. In some embodiments, the PEG linker of L¹, L², and/or L³ enhances the solubility of a conjugate and/or payload conjugated thereto.

In some embodiments, each L¹, L², and L³ is a bivalent linker having a structure represented by formula:

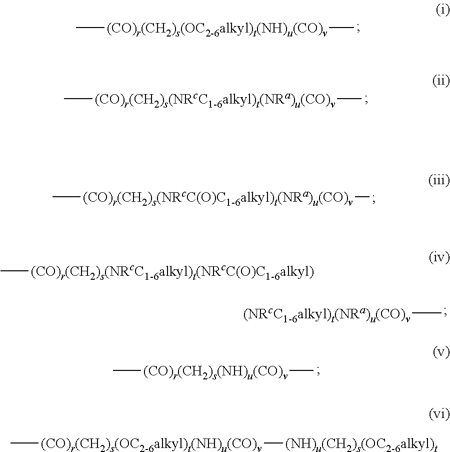

wherein:
R^{a} is, in each instance, independently selected from hydrogen or C₁₋₃ alkyl;
R^{c} is, in each instance, independently selected from hydrogen or C₁₋₃ alkyl;
r is 0 or 1; s is 0 to 10; t is 1 to 10; u is 0 or 1; and v is 0 or 1.

In some embodiments, each L¹, L², and L³ is a bivalent linker having a structure represented by formula:

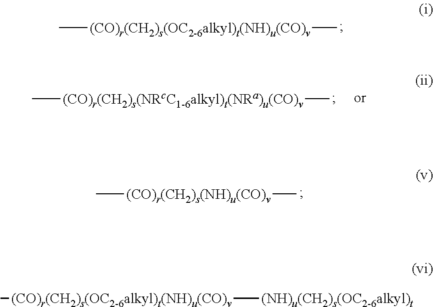

wherein each R^{a} and R^{c} is independently hydrogen or —CH₃;
r is 0 or 1;
s is 1 to 4;
t is 1 to 10;
u is 0 or 1; and
v is 0 or 1.

In some embodiments, each L¹, L², and L³ is independently a bivalent linker of the formula:

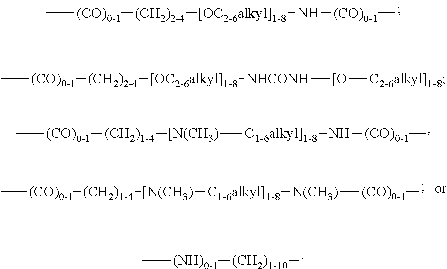

In some embodiments, L¹, L², and/or L³ is:

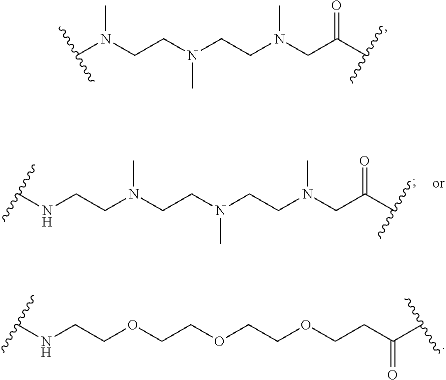 [SMILES: *C(=O)CN(C)CCN(C)CCN(*)C ; *NCCN(C)CCN(C)CCN(C)CC(*)=O ; *NCCOCCOCCOCCC(*)=O]

In some embodiments, L¹ is:

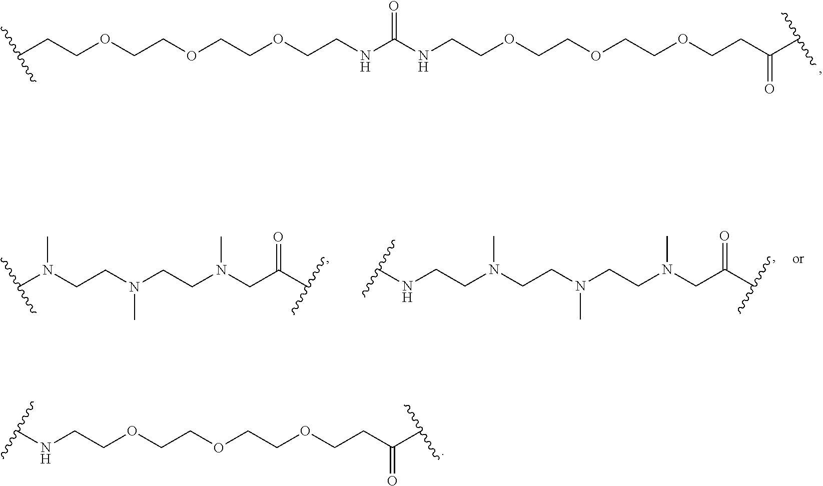 [SMILES: *CCOCCOCCOCCNC(=O)NCCOCCOCCOCCC(*)=O ; *C(=O)CN(C)CCN(C)CCN(*)C ; *NCCN(C)CCN(C)CCN(C)CC(*)=O ; *NCCOCCOCCOCCC(*)=O]

In some embodiments, L³ is:

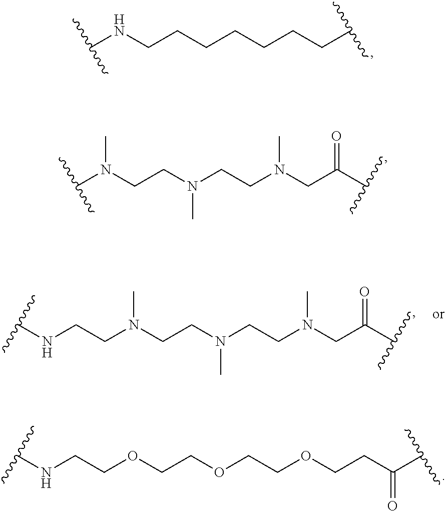 [SMILES: *CCCCCCCN* ; *C(=O)CN(C)CCN(C)CCN(*)C ; *NCCN(C)CCN(C)CCN(C)CC(*)=O ; *NCCOCCOCCOCCC(*)=O]

In some embodiments,
each L¹ is: —CO—(CH₂)₂₋₄—[OCH₂CH₂]₁₋₈—NHCONH—[OCH₂CH₂]₁₋₈, —CO—(CH₂)₂₋₄—(OCH₂CH₂)₁₋₈—NH—, —CO—(CH₂)₂₋₄—(N(CH₃)CH₂CH₂)₁₋₈—N(CH₃)—, or —CO—(CH₂)₂₋₄(N(CH₃)CH₂CH₂)₁₋₈—NH—;
each L² is: —CO—(CH₂)ₛ—(OCH₂CH₂)₁₋₈—NH—; and
each L³ is: —CO—(CH₂)ₛ—(OCH₂CH₂)₁₋₈—NH— or —NH—(CH₂)₁₋₁₀.

In some embodiments, L¹, L², and/or L³ is a bivalent linker comprising 2 to 20 polyethylene glycol groups.

In another aspect, provided herein is a compound of Formula I, II, III, IV, V, VI, VII, VIII, IX, X, XI, or XII, (including sub-formulae thereof) for use in the manufacture of a medicament for treating a disease. In another aspect, provided herein is a method of treating a disease or disorder in a subject in need thereof, the method comprising administering a therapeutically effective amount of a compound disclosed in any one or more of Formula I, II, III, IV, V, VI, VII, VIII, IX, X, XI, or XII, (including sub-formulae thereof), or a pharmaceutically acceptable salt or solvate thereof, to the subject. In some embodiments, the disease or disorder is a hyperproliferative disease or disorder. In some embodiments, provided herein is an antibody-drug conjugate for use as a medicament. In some embodiments, the medicament is for use in the treatment of a disease (e.g., a hyperproliferative disease or disorder). In some embodiments, the hyperproliferative disease or disorder is a cancer.

### Payloads (P)

As used herein, the term “payload” or “therapeutic payload” generally refers to a chemical group, generally a small molecule (i.e., non-protein) group, having therapeutic activity. Preferably, the therapeutic activity is enhanced (e.g., activated) after separation from a cleavable group. In some embodiments, the cleavable group is an enzymatically-cleavable group. In some embodiments, the payload is activated following cleavage by a tumor-associated protein such as neutrophil elastase. A therapeutic payload may be, for example, a drug. In some embodiments, the therapeutic payload is a cytotoxic, cytostatic, or immunomodulatory compound. In some embodiments, the payload is effective in killing or slowing the growth of cancer cells. In some embodiments, the payload is a kinesin spindle protein inhibitor, a camptothecin or a derivative thereof, a CDK9 inhibitor, an auristatin, or a taxane, as described herein. In some embodiments, the payload is a protease-cleavable kinesin spindle protein inhibitor (KSPi), a protease-cleavable camptothecin derivative, a protease-cleavable CDK9 inhibitor, a protease-cleavable auristatin, a protease-cleavable taxane, or a protease-cleavable cytotoxic or immunostimulant payload disclosed herein.

Payloads for use in accordance with the present invention may be, for example, a drug. Thus, conjugates are generally referred to herein as small molecule drug conjugates (SMDCs). A payload can be, for example, a cytotoxic agent or an immunostimulatory agent. Preferably, the therapeutic payload is permeable to cell membranes (e.g., to tumor cell membranes). More preferably, the therapeutic payload is penetrant to tumor cells and produces cytotoxic or antiproliferative effects in a cell. A therapeutic payload may be configured to be released extracellularly in a tumor microenvironment (e.g., by via cleavage of a protease-cleavable linker by an extracellular tumor-associated protein (e.g., neutrophil elastase)). An extracellularly released therapeutic payload, e.g., a tumor-penetrant therapeutic payload, may enter a tumor cell and produce potent cytotoxic or immunostimulant effects.

In some embodiments, the payload is a microtubule toxin (e.g., maytansinoids maytansin, DM1, DM4, DM21, DM23), auristatins (MMAE, MMAF, auristatin F, dolastatin, PF-06380101, amberstatin269, auristatin F-HPA, auristatin W analog, duostatin 5.2, duostatin5, MMAD, SHR152852, Combretastatin A (CBA)), epothilone (epothilone B, epothilone D, ixampra), taxoids (paclitaxel, docetaxel), tubulysins (tubulysin, Tub196, Tub114, Tub201, Tub255, AZ13599185), eribulin and vinca alcaloids (vinorelbine, vinflunine, vinblastine silanol), cytolysine (TAM470), hemiasterlin (SC209, E7974), eribulin.

In some embodiments, the payload is a DNA toxin. In some embodiments, the therapeutic payload is an anthracycline (e.g., doxorubicin, daunorubicin, epiburicin, PNU-159682, panobinostat), a topoisomerase I inhibitor (e.g., AZ′0133, camptothecin, belotecan, irinotecan, topotecan, DXd/DX8951, exatecan, FL-118, SN-38, VIP126), a duocarmycin or an analog thereof (e.g., duocarmycin, duocarmycin-hydroxy benzamide azaindole (DUBA), MED-A/DNAMGBA toxin), a calicheamicin, a DNA cross linking agent (e.g., PBD-dimers-FGX20-75, SC-DR003, SG2000, SG3199, SG1882, FGX2-62, indolino-benzodiazepine dimer-DGN462, DGN549, IGN-P1-, cyclopropylpyrroloindole, isoquinolidinobenzodiazepine-D211) bleomycin A2, dactinomycin, and mitomycin C.

In some embodiments, the payload is a transcription toxin. In some embodiments, the therapeutic payload is an amatoxin (e.g., targeting RNA polymerase II, e.g., amanitin), thailanstatin A (e.g., targeting spliceosome), an oxidative phosphorylation inhibitor (e.g., oligomycin), a protein kinase inhibitor (e.g., an inhibitor of protein kinase B (Akt) such as ipatasertib), an EGFR inhibitor (e.g., erlotinib), a VEGFR inhibitor, (e.g., sorafenib, sunitinib, bevacizumab, Lenvatinib, vandetanib, pazopanib, axitinib, cabozantinib, regorafenib, nintedanib, apatinib), a PDGFR inhibitor, a dihydrofolate reductase (DHFR) inhibitor, e.g., methotrexate, aminopterin, a histone deacetylase inhibitor (e.g., HST746AA1), or a kinesin spindle protein inhibitor (KSPi).

In some instances, the payload is a microtubule toxin, DNA toxin, transcription toxin, or an immune stimulator. In some instances, the microtubule toxin is a maytansinoid, auristatin, epithilone, taxoid, tubulysin, eribulin alkaloid, vinca alkaloid, eribulin, or any combination thereof. In some instances, the DNA toxin is an anthracycline, topoisomerase I inhibitor, duacarmycin or analogs thereof, calichearmicins, DNA cross linking agents, bleomycin A2, dactinomucin, mitomycin C, or any combination thereof. In some instances, the transcription toxin is an amnatoxin, thailanstatin A, oxidating phosphorylation inhibitor, protein kinase inhibitor, dihydrofolate reductase (DHFR) inhibitor, or histone deactylase inhibitor.

In some preferred embodiments, provided herein are compounds with payloads such as camptothecin derivatives, auristatin derivatives, CDK9/PTEFb derivatives, kinesin spindle protein inhibitor derivatives. In some embodiments, a payload (P) of Formula I, II, III, IV, V, VI, VII, VIII, IX, X, XI, or XII is represented by one of the following structures:

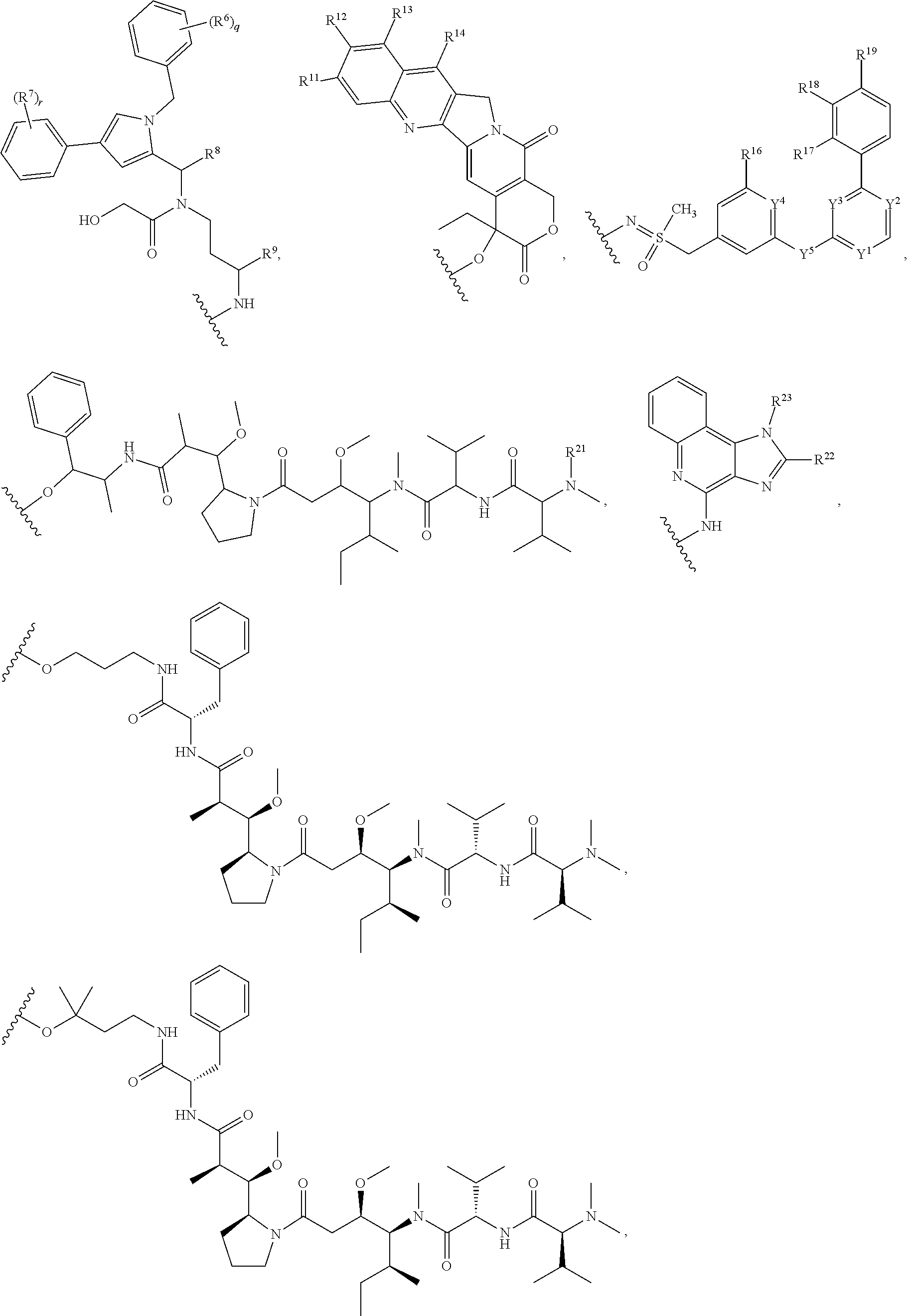

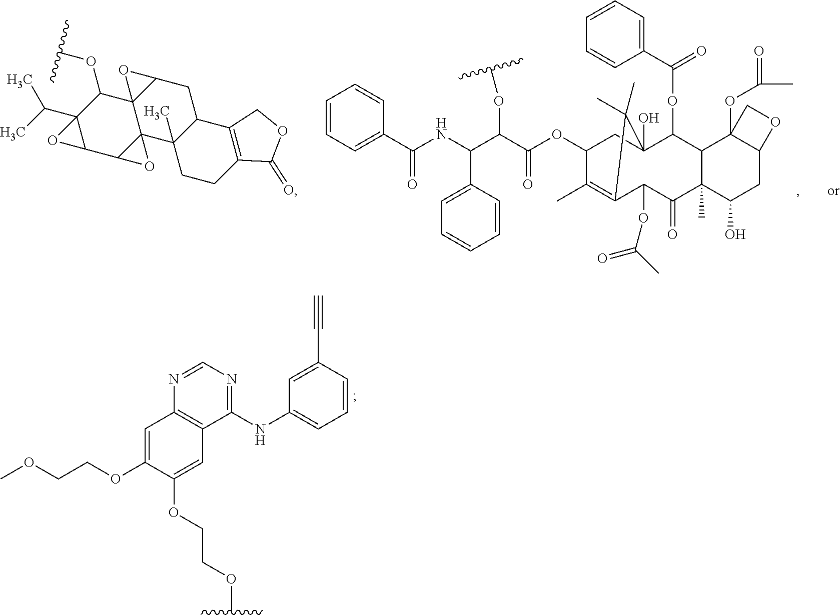

or a stereoisomer thereof, or a pharmaceutically acceptable salt thereof, wherein: each R⁶ and R⁷ is independently hydrogen, halogen, CN, —C₁₋₆ alkyl, or C₁. haloalkyl;
R⁸ is hydrogen, halogen, CN, —C₁₋₆ alkyl, C₁₋₆ haloalkyl, C₃₋₆ cycloalkyl, or 5- to 7-membered heterocycloalkyl;
R⁹ is hydrogen, halogen, CN, C₁₋₆ alkyl, —CH₂C(O)NH₂, —CH₂C(O)NHC₁₋₆ alkyl, —CH₂C(O)N(C₁₋₆ alkyl)₂, —C(O)NH₂, —C(O)NHC₁₋₆ alkyl, —C(O)N(C₁₋₆ alkyl)₂, —C(O)NHC₁₋₆ alkyl-C(O)NHC₁₋₆ alkyl, —C(O)NHC₁₋₄ alkyl-NHC(O)C₁₋₆ alkyl, —NH₂, —NHC₁₋₆ alkyl, —N(C₁₋₆ alkyl)₂, —NHC(O)C₁₋₆ alkyl, —OH, or —OC₁₋₆ alkyl; wherein each C₁₋₆ alkyl is substituted with 0-5 R¹⁰;
R¹⁰ is in each instance independently selected from the group consisting of hydrogen, halogen, CN, —COOH, —CONH₂, —NH₂, —NHCH₃, —N(CH₃)₂, —OH, and —OCH₃;
R¹¹ and R¹² are each independently hydrogen, halogen, C₁₋₆ alkyl, C₁₋₆ haloalkyl, or —OH; or R¹¹ and R¹², taken together, form a 5- or 6-membered heterocycle;
R¹³ and R¹⁴ are each independently hydrogen, C₁₋₆ alkyl, or C₁₋₆ alkylamine; or R¹³ and R¹⁴, taken together, form a C6 carbocycle substituted with —N(R¹⁵)₂;
each R¹⁵ is independently hydrogen, C₁₋₆ alkyl, —C(O)C₁₋₆ alkyl, —C(O)NHC₁₋₆ alkyl or —C(O)OC₁₋₆ alkyl; wherein the C₁₋₆ alkyl of R¹⁵ is optionally substituted with halogen, hydroxy, phenyl, or heteroaryl; or R¹⁵ is a cleavable prodrug group;
R¹⁶ is hydrogen, halogen, C₁₋₆ alkyl, C₁₋₆ haloalkyl, —OH, —OCH₃, or —OCF₃;
R¹⁷ is hydrogen, halogen, C₁₋₆ alkyl, C₁₋₆ haloalkyl, —OH, —OCH₃, or —OCF₃; or R¹⁶ and R¹⁷, taken together, form a heteroalkylene group of the formula: —O—C₂₋₁₀ alkylene-O—, —NH—C₂₋₁₀ alkylene-O—, or —NH—C₂₋₁₀ alkylene-NH—; wherein the heteroalkylene group is optionally substituted with R²¹;
R¹⁸ is hydrogen, halogen, C₁₋₆ alkyl, C₁₋₆ haloalkyl, —OH, —OCH₃, or —OCF₃; or R¹⁶ and R¹⁸, taken together, form a heteroalkylene group of the formula: —O—C2-10 alkylene-O—, —NH—C₂₋₁₀ alkylene-O—, or —NH—C₂₋₁₀ alkylene-NH—; wherein the heteroalkylene group is optionally substituted with R²⁰;
R⁹ is hydrogen, halogen, C₁₋₆ alkyl, C₁₋₆ haloalkyl, —OH, —OCH₃, or —OCF₃;
R²⁰ is hydrogen, halogen, C₁₋₆ alkyl, C₁₋₆ haloalkyl, or OH;
R²¹ is hydrogen or methyl;
R²² is hydrogen or C₁₋₆ alkyl, wherein the C₁₋₆ alkyl is unsubstituted or substituted with R²⁴;
R²³ is hydrogen, C₁₋₆ alkyl, or benzyl, wherein the C₁₋₆ alkyl or benzyl is unsubstituted or substituted with one, two, or three R²³ groups;
R²⁴ is —OH, —O(C₁₋₆ alkyl), —NH₂, —NH(C₁₋₆ alkyl), —N(C₁₋₆ alkyl)₂, —SH, or —S(C₁₋₆ alkyl);
R²⁵ is —OH, —O(C₁₋₆ alkyl), —NH₂, —NH(C₁₋₆ alkyl), —N(C₁₋₆ alkyl)₂, —NHS(O)₂(C₁₋₆ alkyl), C₁₋₆ alkyl, C₁₋₆ aminoalkyl, or OCH₂CH₂NHC(O)(C₁₋₆ aminoalkyl);
each Y¹, Y², Y³, and Y⁴ is independently —CH, —CF, or N;
Y⁵ is CH₂, NH, or O;
q is 0, 1, 2, 3, 4, or 5; and
r is 0, 1, 2, 3, 4, or 5.

In some embodiments, the payload (P) of Formula I, II, III, IV, V, VI, VII, VIII, IX, X, XI, or XII is:

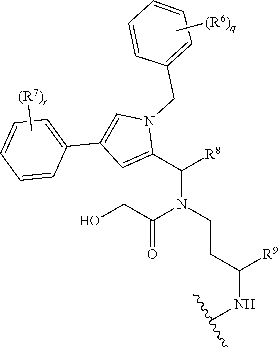

or a stereoisomer thereof, or a pharmaceutically acceptable salt thereof, wherein:
each R⁶ and R⁷ is independently hydrogen, halogen, CN, —C₁₋₆ alkyl, or C₁₋₆ haloalkyl;
R⁸ is hydrogen, halogen, CN, —C₁₋₆ alkyl, C₁₋₆ haloalkyl, C₃₋₆ cycloalkyl, or 5- to 7-membered heterocycloalkyl;
R⁹ is hydrogen, halogen, CN, C₁₋₆ alkyl, —C(O)NH₂, —C(O)NHC₁₋₆ alkyl, —C(O)N(C₁₋₆ alkyl)₂, —C(O)NHC₁₋₆ alkyl-C(O)NHC₁₋₆ alkyl, —C(O)NHC₁₋₆ alkyl-NHC(O)C₁₋₆ alkyl, —NH₂, —NHC₁₋₆ alkyl, —N(C₁₋₆ alkyl)₂, —NHC(O)C₁₋₆ alkyl, —OH, or —OC₁₋₆ alkyl; wherein each C₁₋₆ alkyl is substituted with 0-5 R¹⁰;
R¹⁰ is in each instance independently selected from the group consisting of hydrogen, halogen, CN, —COOH, —CONH₂, —NH₂, —NHCH₃, —N(CH₃)₂, —OH, and —OCH₃;
q is 0, 1, 2, 3, 4, or 5; and
r is 0, 1, 2, 3, 4, or 5.

In some embodiments, the payload (P) of Formula I, II, III, IV, V, VI, VII, VIII, IX, X, XI, or XII is:

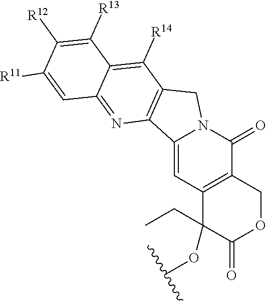

or a stereoisomer thereof, or a pharmaceutically acceptable salt thereof, wherein:
R¹¹ and R¹² are each independently hydrogen, halogen, C₁₋₆ alkyl, C₁₋₆ haloalkyl, or —OH; or R¹¹ and R¹², taken together, form a 5- or 6-membered heterocycle;
R¹³ and R¹⁴ are each independently hydrogen, C₁₋₆ alkyl, or C₁₋₆ alkylamine; or R¹³ and R¹⁴, taken together, form a C₆ carbocycle substituted with —N(R¹⁵)₂;
each R¹⁵ is independently hydrogen, C₁₋₆ alkyl, —C(O)C₁₋₆ alkyl, —C(O)NHC₁₋₆ alkyl or —C(O)OC₁₋₆ alkyl; wherein the C₁₋₆ alkyl of R¹⁵ is optionally substituted with halogen, hydroxy, phenyl, or heteroaryl; or R¹⁵ is a cleavable prodrug group (e.g, a benzyl ester).

In some embodiments, the payload (P) of Formula I, II, III, IV, V, VI, VII, VIII, IX, X, XI, or XII is:

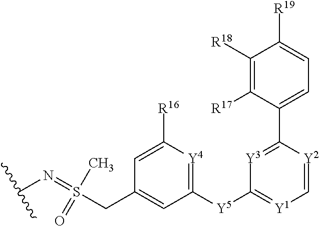

or a stereoisomer thereof, or a pharmaceutically acceptable salt thereof, wherein:
R¹⁶ is hydrogen, halogen, C₁₋₆ alkyl, C₁₋₆ haloalkyl, —OH, —OCH₃, or —OCF₃;
R¹⁷ is hydrogen, halogen, C₁₋₆ alkyl, C₁₋₆ haloalkyl, —OH, —OCH₃, or —OCF₃; or R¹⁶ and R¹⁷, taken together, form a heteroalkylene group of the formula: —O—C₂₋₁₀ alkylene-O—, —NH-C₂₋₁₀ alkylene-O—, or —NH—C₂₋₁₀ alkylene-NH—; wherein the heteroalkylene group is optionally substituted with R²¹;
R¹⁸ is hydrogen, halogen, C₁₋₆ alkyl, C₁₋₆ haloalkyl, —OH, —OCH₃, or —OCF₃; or R¹⁶ and R¹⁸, taken together, form a heteroalkylene group of the formula: —O—C₂₋₁₀ alkylene-O—, —NH—C₂₋₁₀ alkylene-O—, or —NH—C₂₋₁₀ alkylene-NH—; wherein the heteroalkylene group is optionally substituted with R²¹;
R¹⁹ is hydrogen, halogen, C₁₋₆ alkyl, C₁₋₆ haloalkyl, —OH, —OCH₃, or —OCF₃;
R²¹ is hydrogen, halogen, C₁₋₆ alkyl, C₁₋₆ haloalkyl, or OH;
each Y¹, Y², Y³, and Y⁴ is independently —CH, —CF, or N; and
Y is CH₂, NH, or O.

In some embodiments, the payload (P) of Formula I, II, III, IV, V, VI, VII, VIII, IX, X, XI, or XII is:

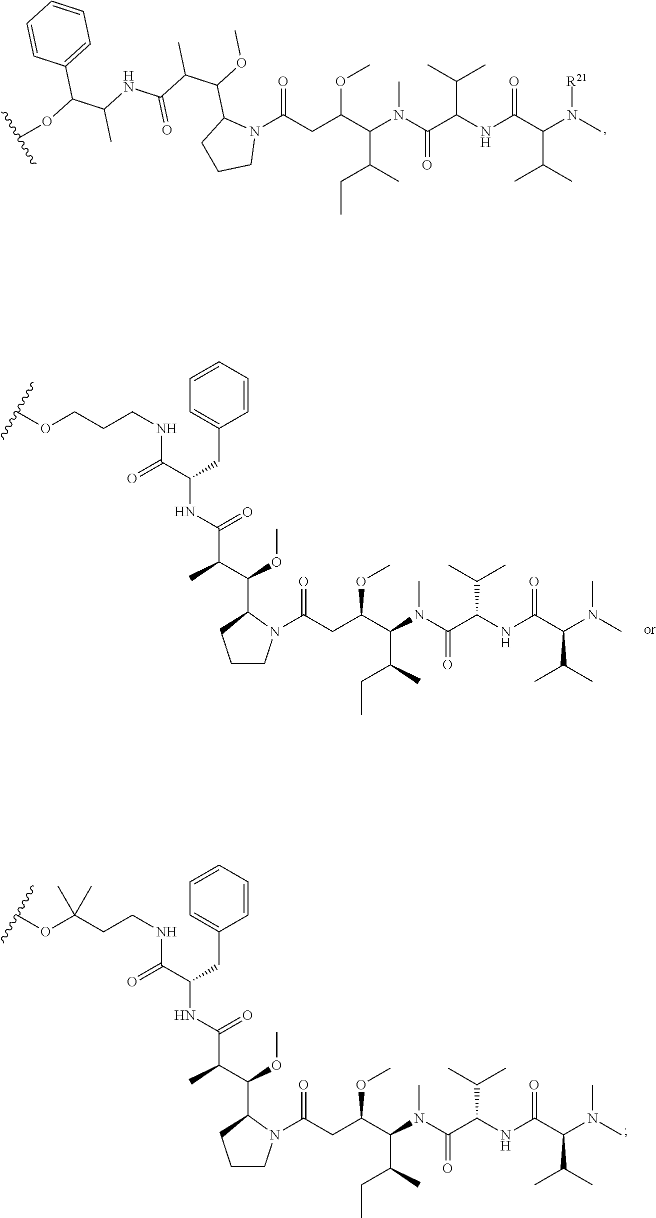

or a stereoisomer thereof, or a pharmaceutically acceptable salt thereof, wherein:
R²¹ is hydrogen or methyl.

In some embodiments, SIL is a self-immolative linker group. In some embodiments, SIL is C₂₋₁₈ heteroalkylene self-immolative linker group. In some embodiments, SIL is —(NH)₀₋₁—(O)₀₋₁(CR^{d}₂)₁₋₄(CO)₀₋₁—*; wherein each R^{d} is independently hydrogen or —CH₃; and * represents the bond to P. In some embodiments, SIL is —(NH—(CR^{d}₂)₁₋₄—CO*. In some embodiments, SIL is:

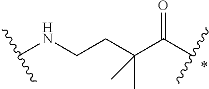 [SMILES: *NCCC(C)(C)C(*)=O]

wherein * represents the bond to P.

In some embodiments, R²¹ is hydrogen. In some embodiments, R²¹ is —CH₃. In some embodiments, P is:

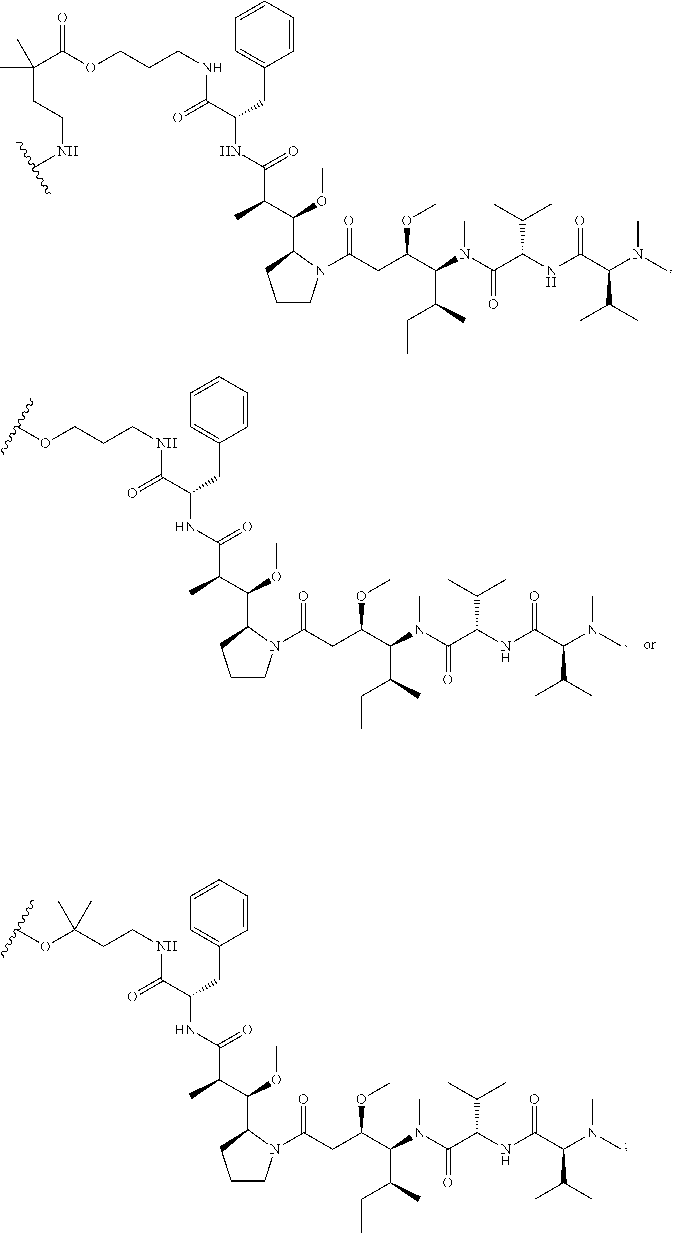 [SMILES: *NCCC(C)(C)C(=O)OCCCNC(=O)[C@H](Cc1ccccc1)NC(=O)[C@H](C)[C@@H](OC)[C@@H]1CCCN1C(=O)C[C@@H](OC)[C@H]([C@@H](C)CC)N(C)C(=O)[C@@H](NC(=O)[C@H](C(C)C)N(C)C)C(C)C ; *OCCCNC(=O)[C@H](Cc1ccccc1)NC(=O)[C@H](C)[C@@H](OC)[C@@H]1CCCN1C(=O)C[C@@H](OC)[C@H]([C@@H](C)CC)N(C)C(=O)[C@@H](NC(=O)[C@H](C(C)C)N(C)C)C(C)C ; *OC(C)(C)CCNC(=O)[C@H](Cc1ccccc1)NC(=O)[C@H](C)[C@@H](OC)[C@@H]1CCCN1C(=O)C[C@@H](OC)[C@H]([C@@H](C)CC)N(C)C(=O)[C@@H](NC(=O)[C@H](C(C)C)N(C)C)C(C)C]

or a pharmaceutically acceptable salt thereof.

In some embodiments, the payload (P) of Formula I, II, III, IV, V, VI, VII, VIII, IX, X, XI, or XII is:

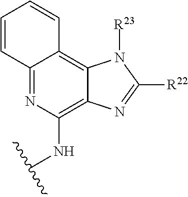

or a stereoisomer thereof, or a pharmaceutically acceptable salt thereof, wherein:
R²² is hydrogen or C₁₋₆ alkyl, wherein the C₁₋₆ alkyl is unsubstituted or substituted with R²⁴;
R²³ is hydrogen, C₁₋₆ alkyl, or benzyl, wherein the C₁₋₆ alkyl or benzyl is unsubstituted or substituted with one, two, or three R²¹ groups;
R²⁴ is —OH, —O(C₁₋₆ alkyl), —NH₂, —NH(C₁₋₆ alkyl), —N(C₁₋₆ alkyl)₂, —SH, or —S(C₁. alkyl);
R²⁵ is —OH, —O(C₁₋₆ alkyl), —NH₂, —NH(C₁₋₆ alkyl), —N(C₁₋₆ alkyl)₂, —NHS(O)₂(C₁. alkyl), C₁₋₆ alkyl, C₁₋₆ aminoalkyl, or OCH₂CH₂NHC(O)(C₁₋₆ aminoalkyl).

Examples of payloads (P) include, but are not limited to:

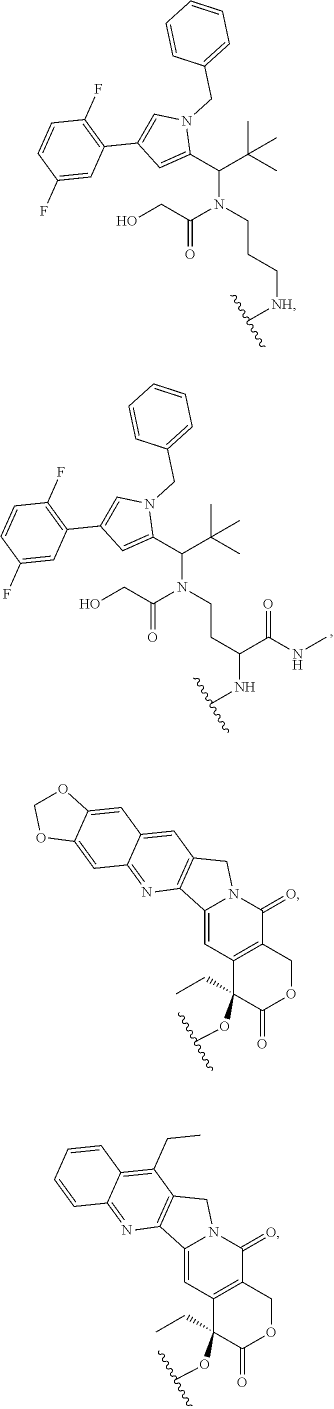 [SMILES: *NCCCN(C(=O)CO)C(c1cc(-c2cc(F)ccc2F)cn1Cc1ccccc1)C(C)(C)C ; *NC(CCN(C(=O)CO)C(c1cc(-c2cc(F)ccc2F)cn1Cc1ccccc1)C(C)(C)C)C(=O)NC ; *O[C@]1(CC)C(=O)OCc2c1cc1n(c2=O)Cc2cc3cc4c(cc3nc2-1)OCO4 ; *O[C@]1(CC)C(=O)OCc2c1cc1n(c2=O)Cc2c-1nc1ccccc1c2CC]

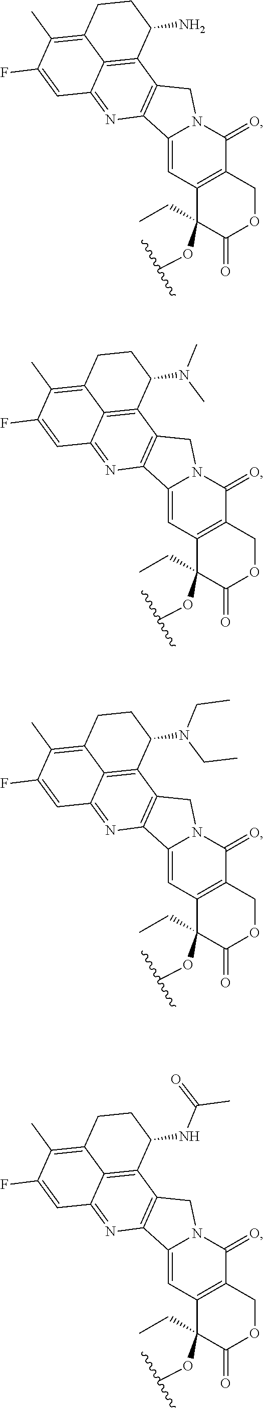 [SMILES: *O[C@]1(CC)C(=O)OCc2c1cc1n(c2=O)Cc2c-1nc1cc(F)c(C)c3c1c2[C@@H](N)CC3 ; *O[C@]1(CC)C(=O)OCc2c1cc1n(c2=O)Cc2c-1nc1cc(F)c(C)c3c1c2[C@@H](N(C)C)CC3 ; *O[C@]1(CC)C(=O)OCc2c1cc1n(c2=O)Cc2c-1nc1cc(F)c(C)c3c1c2[C@@H](N(CC)CC)CC3 ; *O[C@]1(CC)C(=O)OCc2c1cc1n(c2=O)Cc2c-1nc1cc(F)c(C)c3c1c2[C@@H](NC(C)=O)CC3]

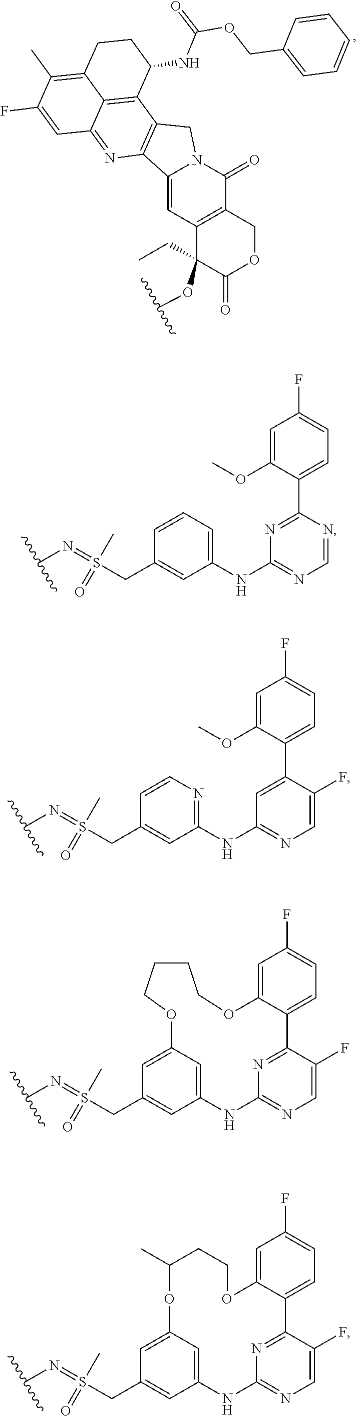 [SMILES: *O[C@]1(CC)C(=O)OCc2c1cc1n(c2=O)Cc2c-1nc1cc(F)c(C)c3c1c2[C@@H](NC(=O)OCc1ccccc1)CC3 ; *N=S(C)(=O)Cc1cccc(Nc2ncnc(-c3ccc(F)cc3OC)n2)c1 ; *N=S(C)(=O)Cc1ccnc(Nc2cc(-c3ccc(F)cc3OC)c(F)cn2)c1 ; *N=S(C)(=O)Cc1cc2cc(c1)OCCCCOc1cc(F)ccc1-c1nc(ncc1F)N2 ; *N=S(C)(=O)Cc1cc2cc(c1)OC(C)CCOc1cc(F)ccc1-c1nc(ncc1F)N2]

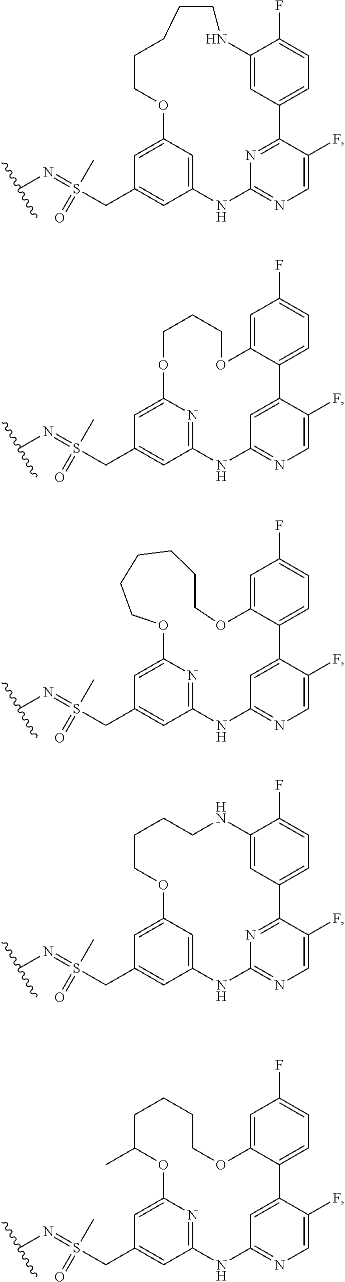 [SMILES: *N=S(C)(=O)Cc1cc2cc(c1)OCCCCCNc1cc(ccc1F)-c1nc(ncc1F)N2 ; *N=S(C)(=O)Cc1cc2nc(c1)OCCCOc1cc(F)ccc1-c1cc(ncc1F)N2 ; *N=S(C)(=O)Cc1cc2nc(c1)OCCCCCCOc1cc(F)ccc1-c1cc(ncc1F)N2 ; *N=S(C)(=O)Cc1cc2cc(c1)OCCCCNc1cc(ccc1F)-c1nc(ncc1F)N2 ; *N=S(C)(=O)Cc1cc2nc(c1)OC(C)CCCCOc1cc(F)ccc1-c1cc(ncc1F)N2]

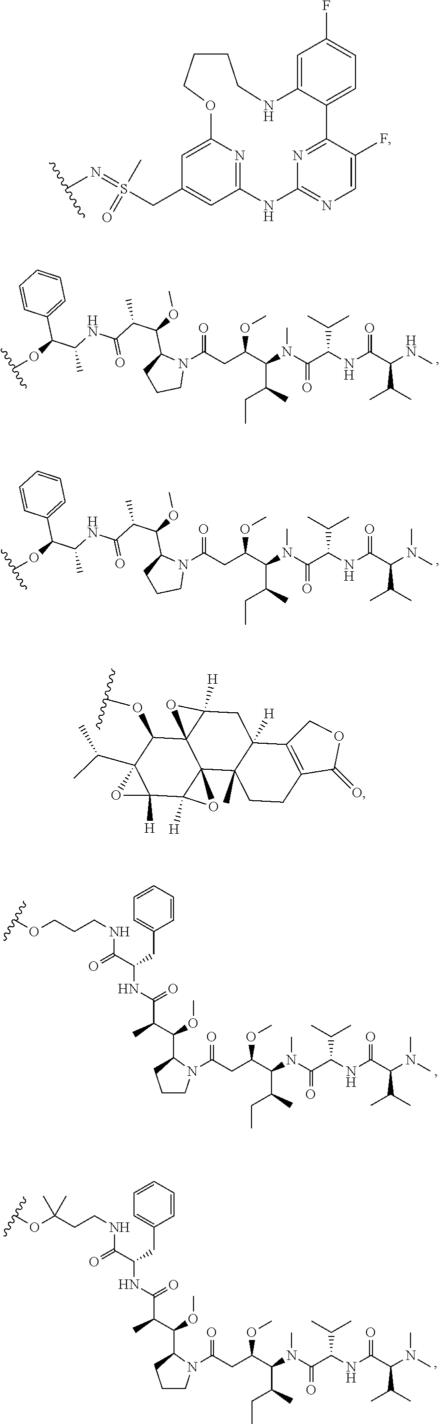

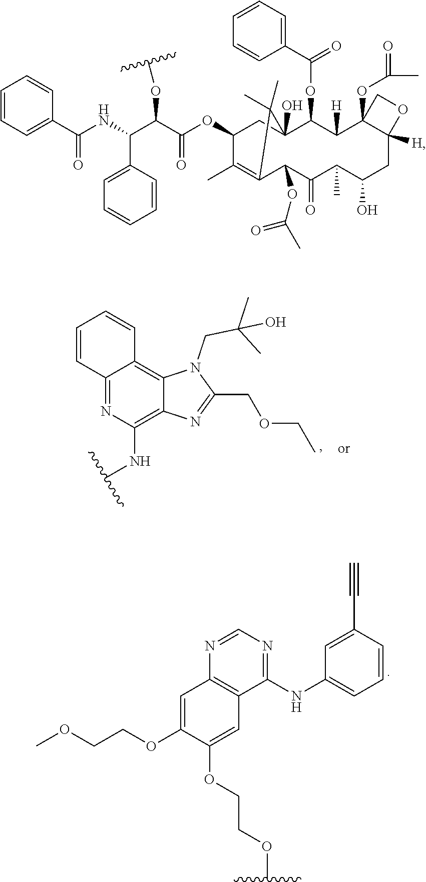

In some embodiments, the conjugate comprises a payload (P) having the structure:

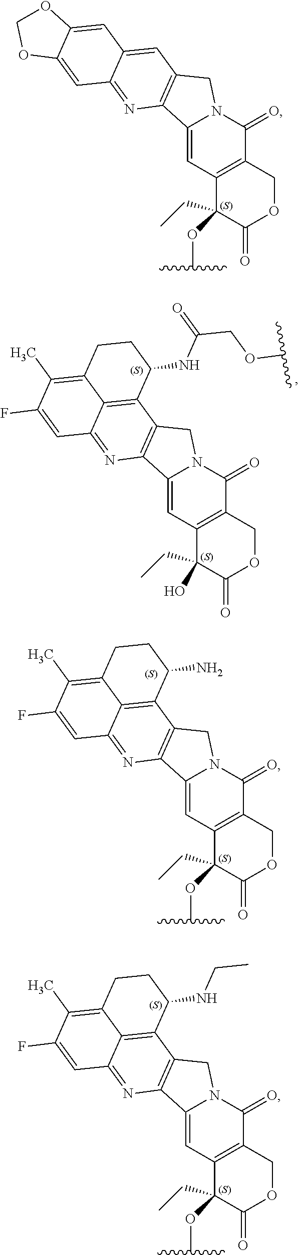

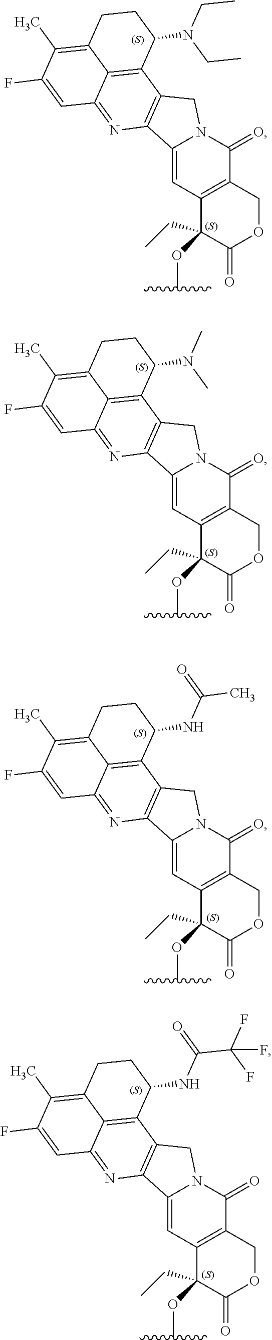

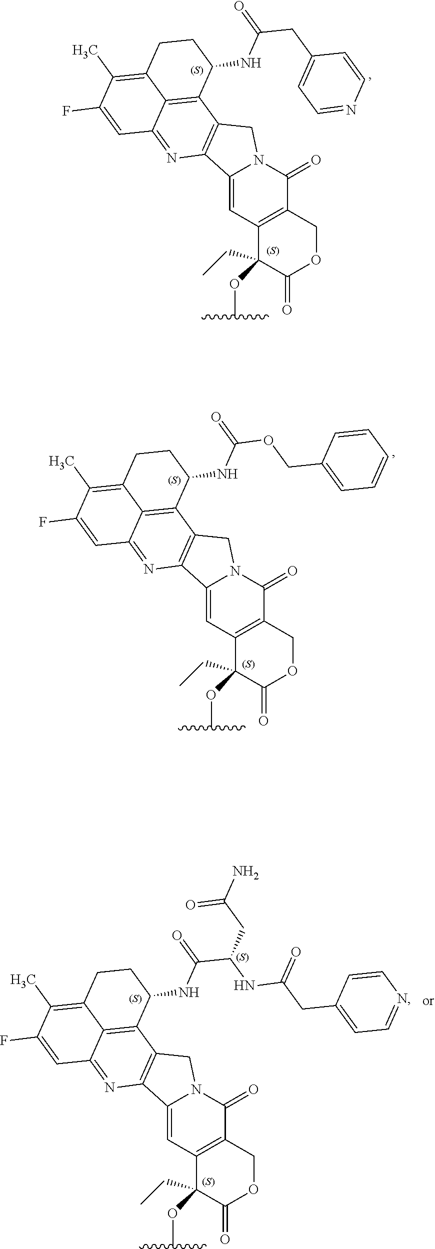

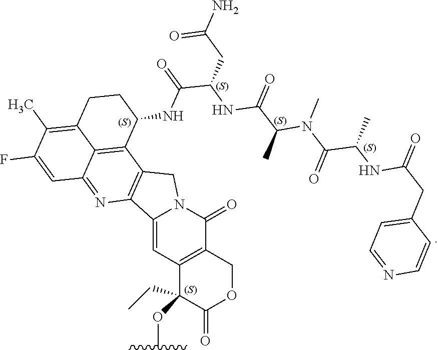

In some embodiments, provided herein is a small-molecule drug conjugate that is:

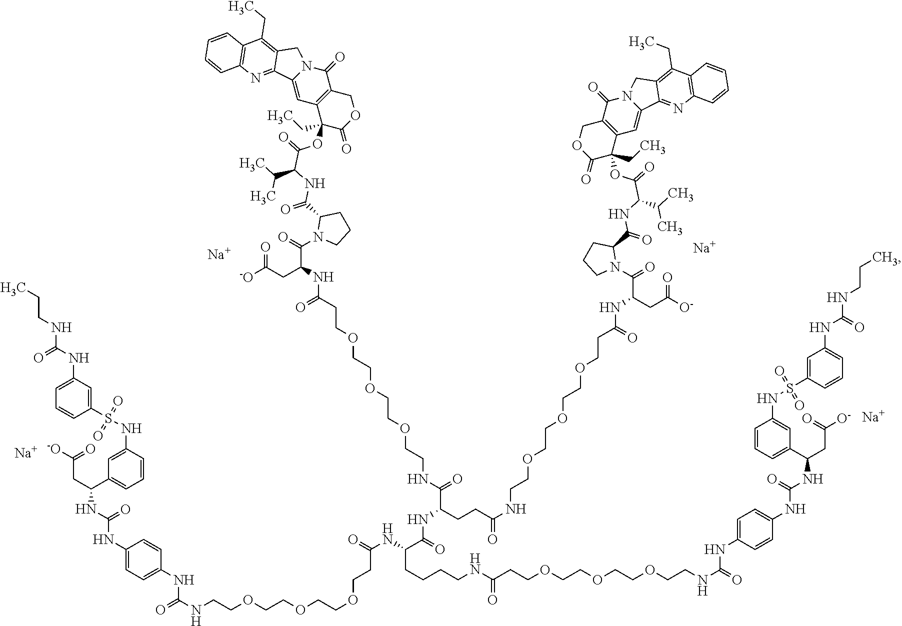

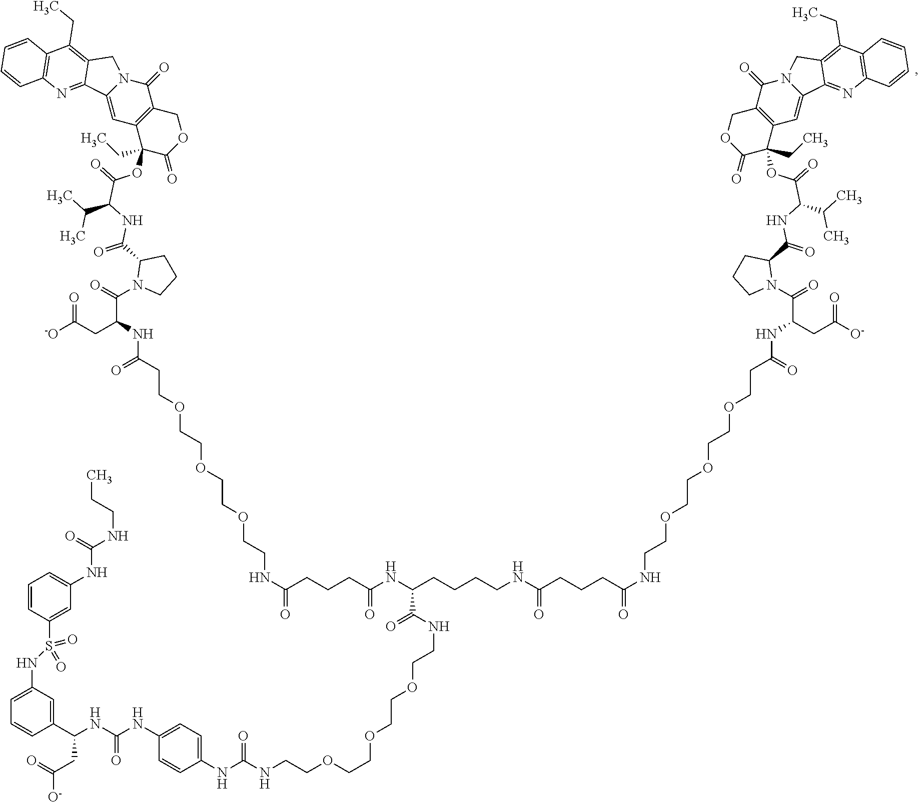

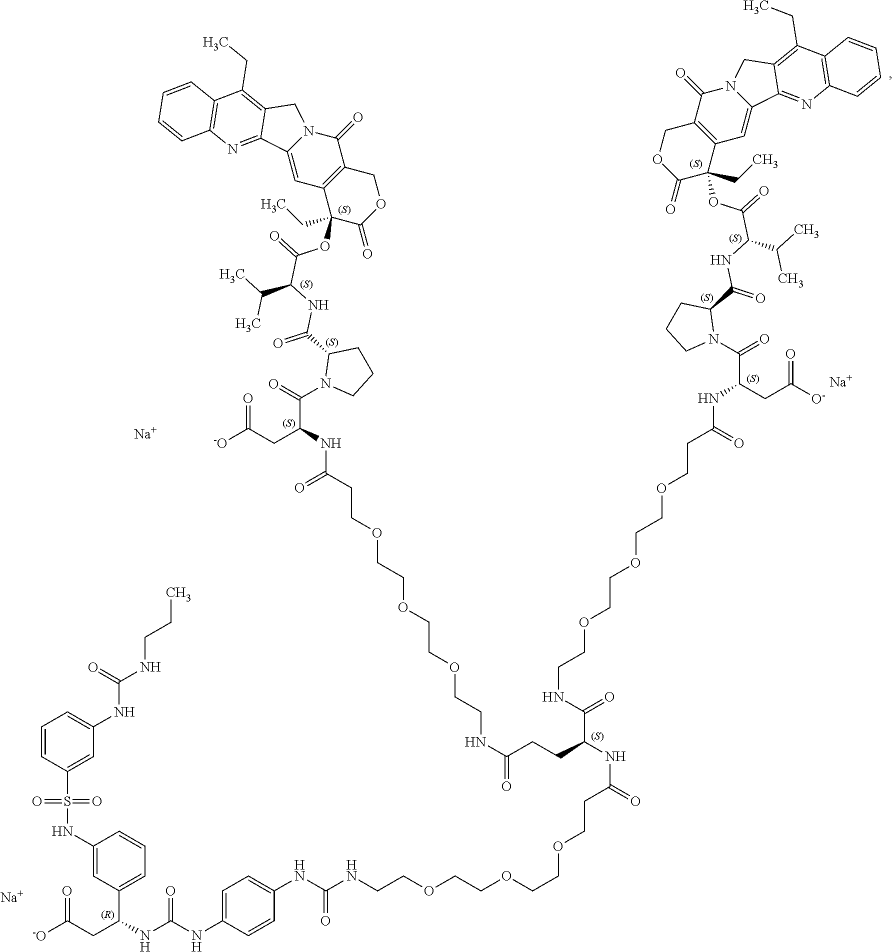

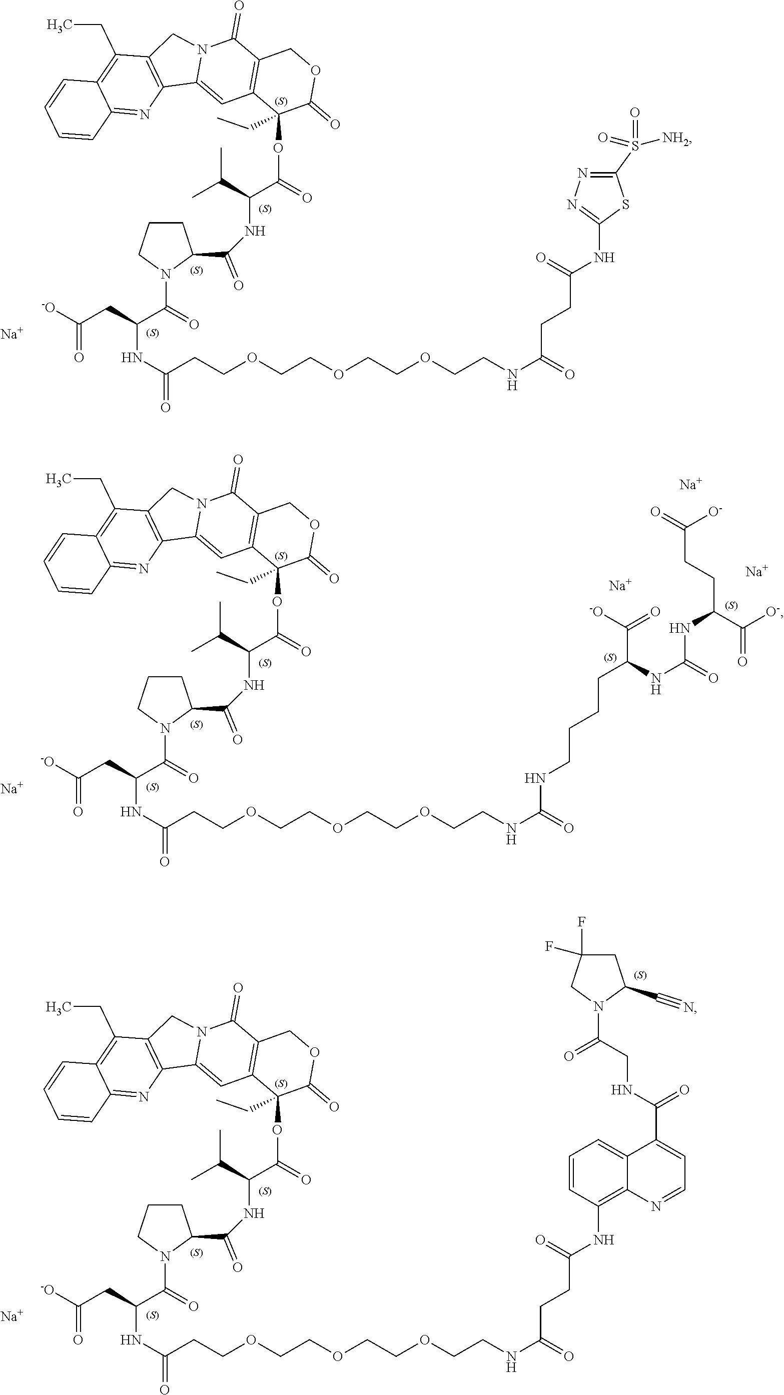

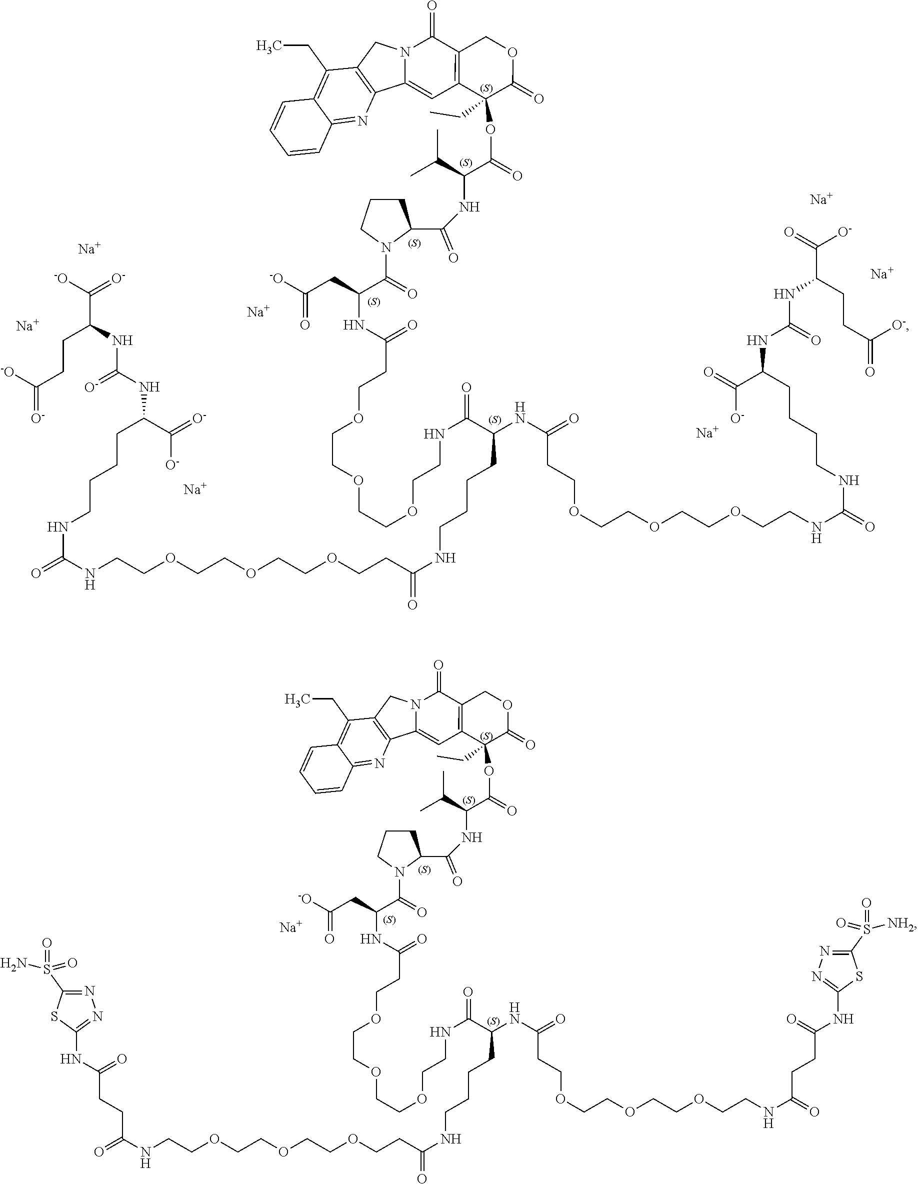

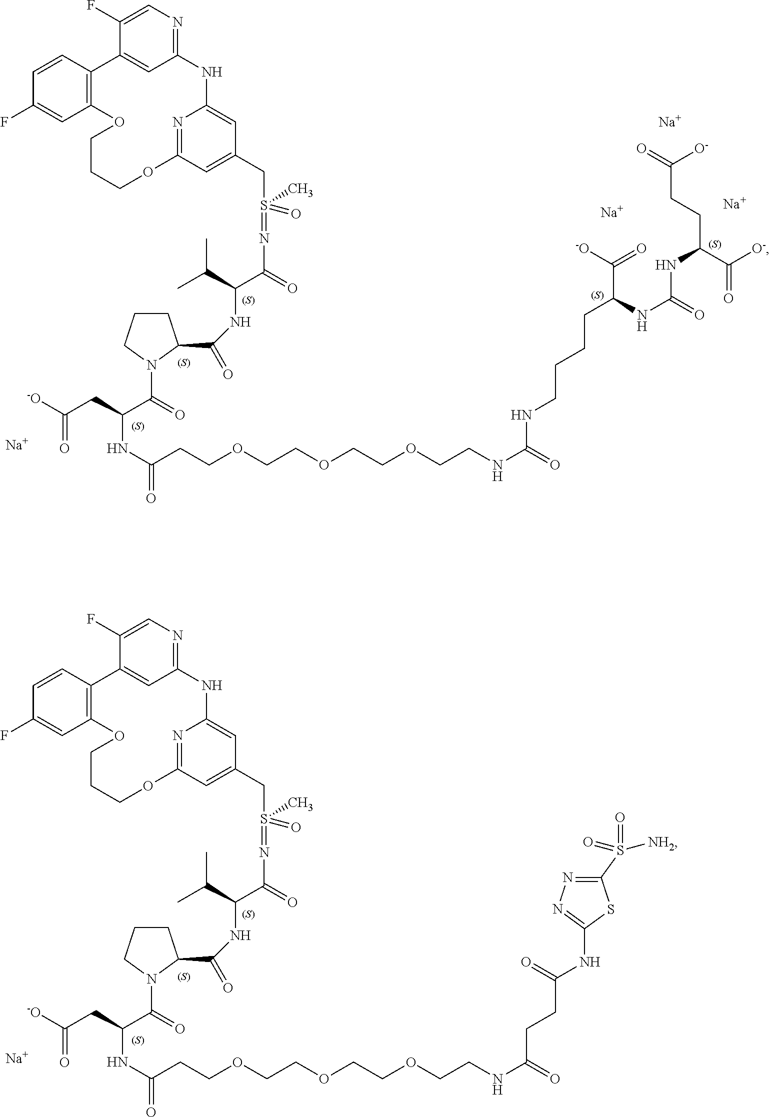

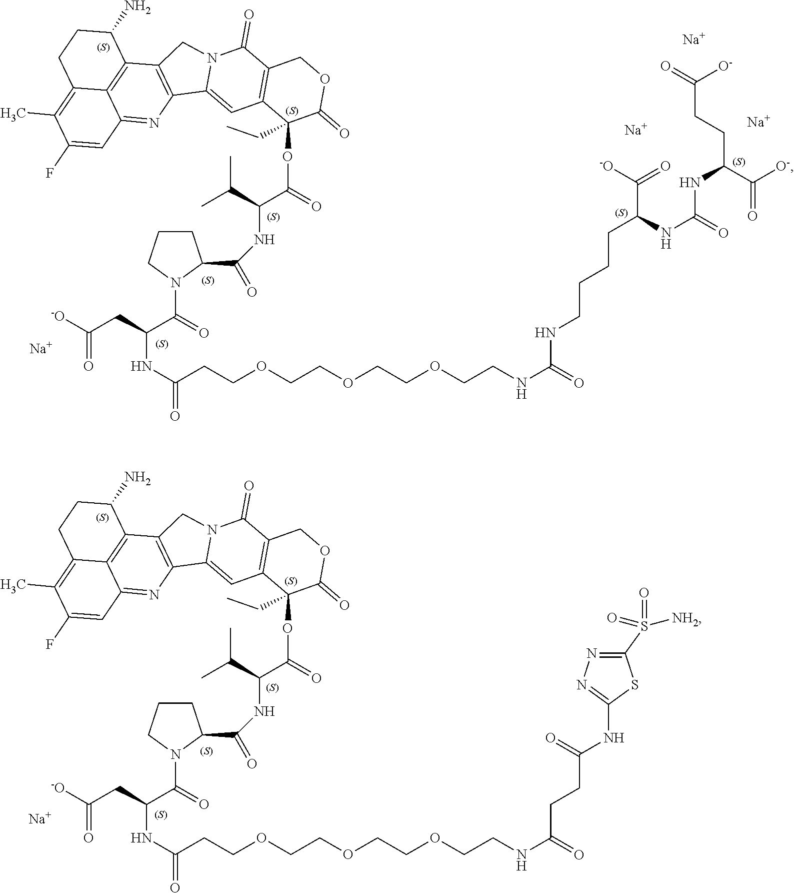

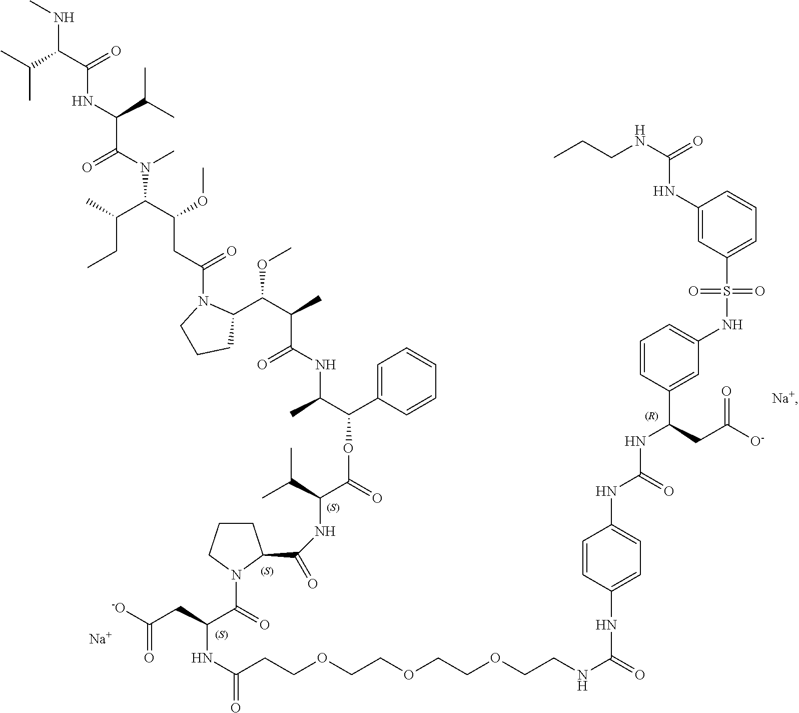

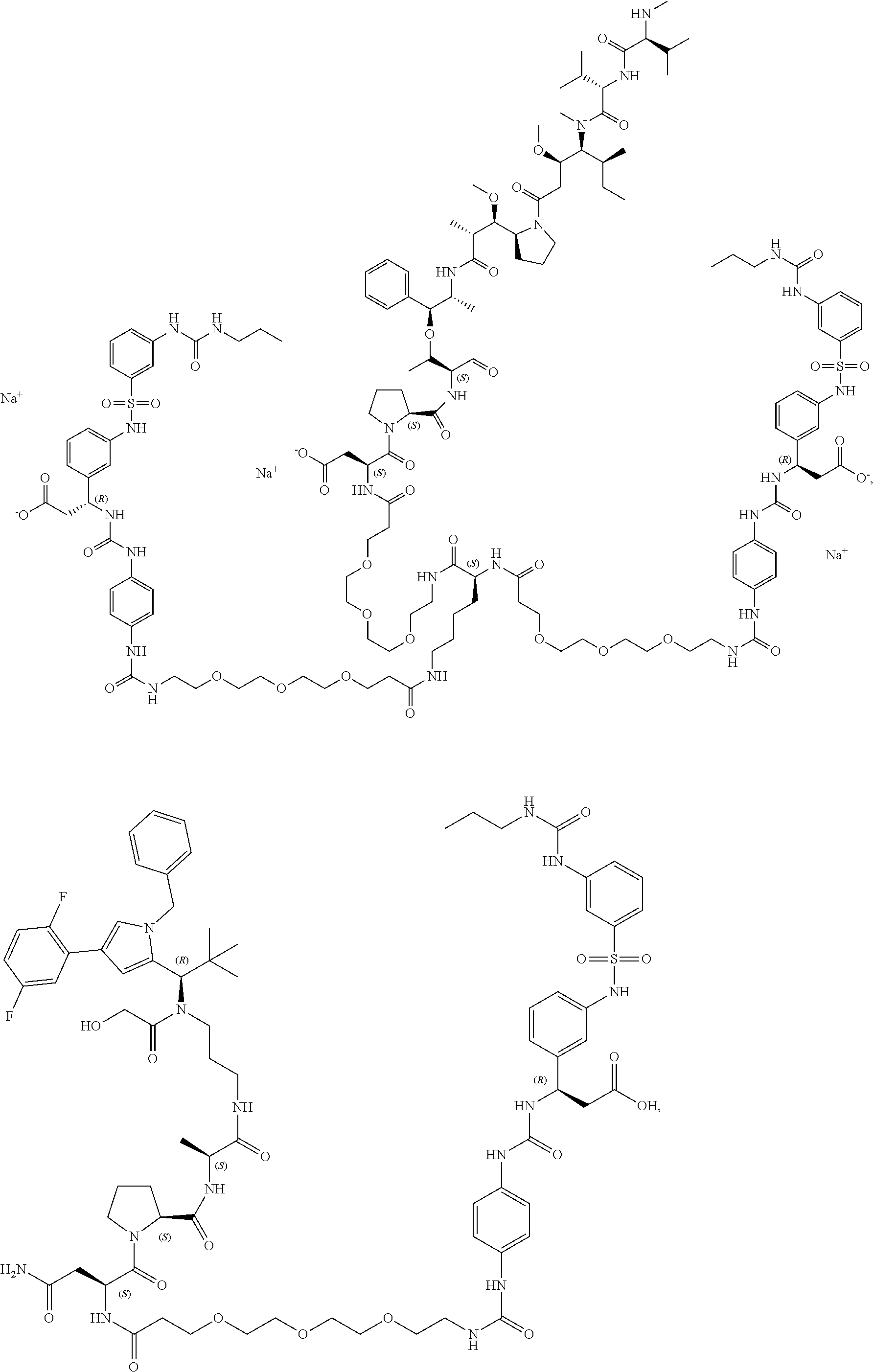

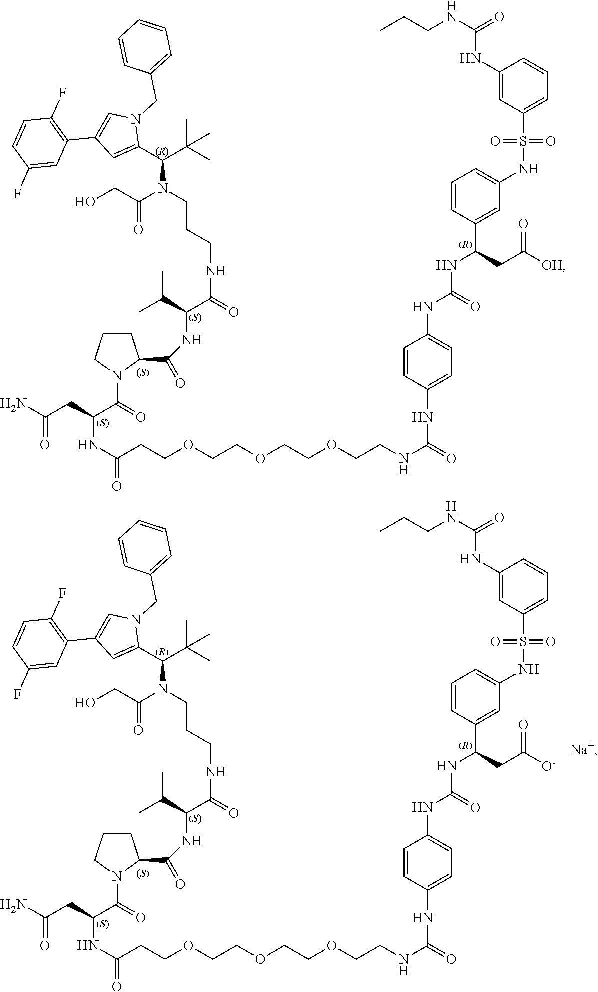

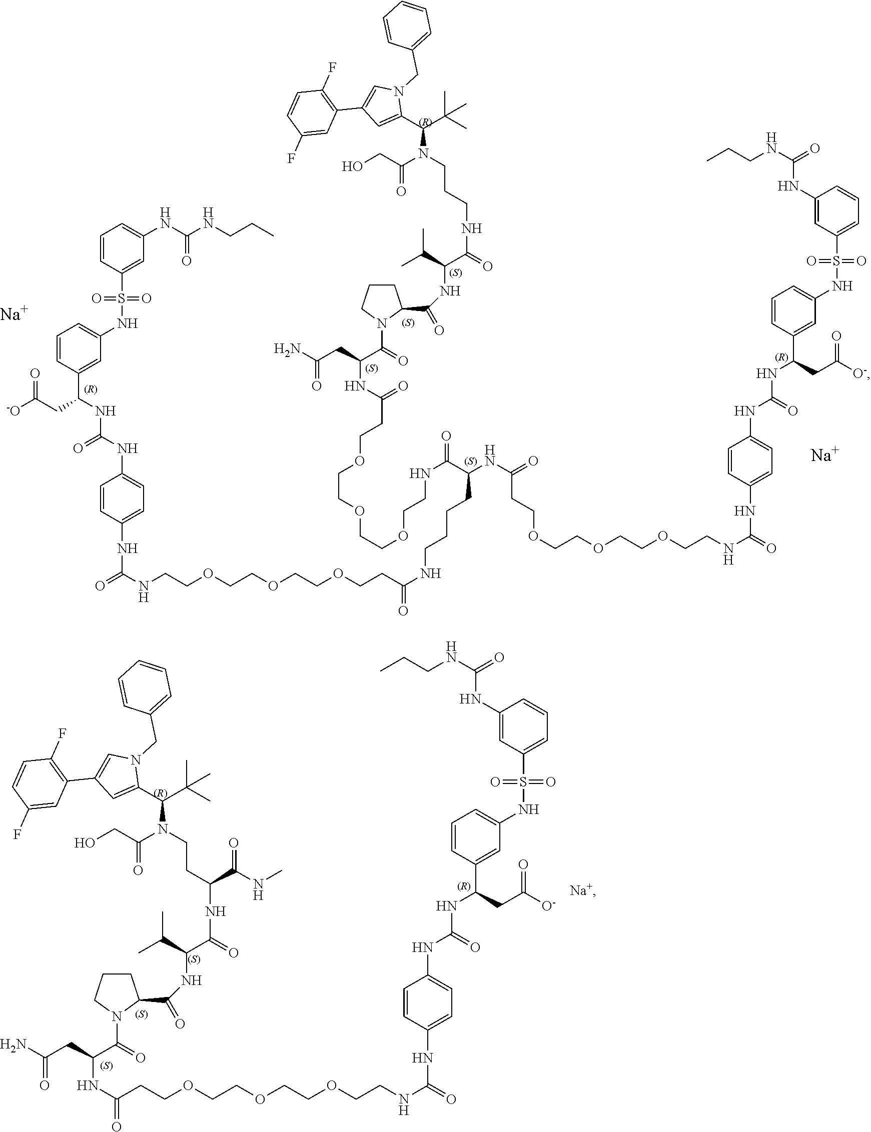

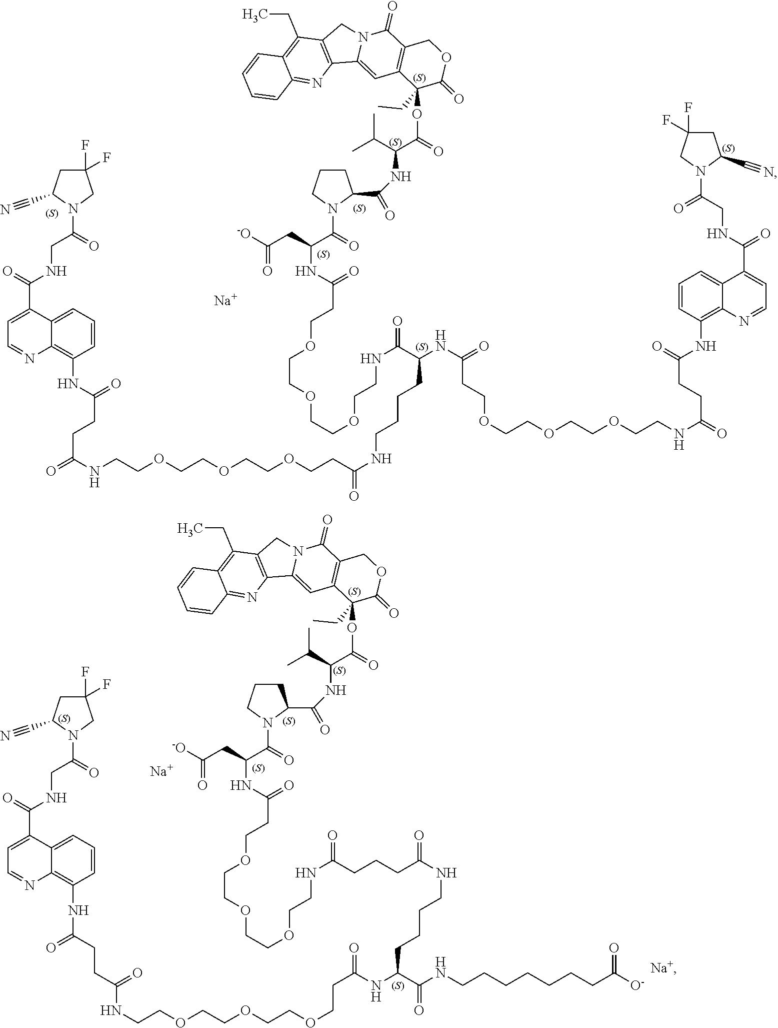

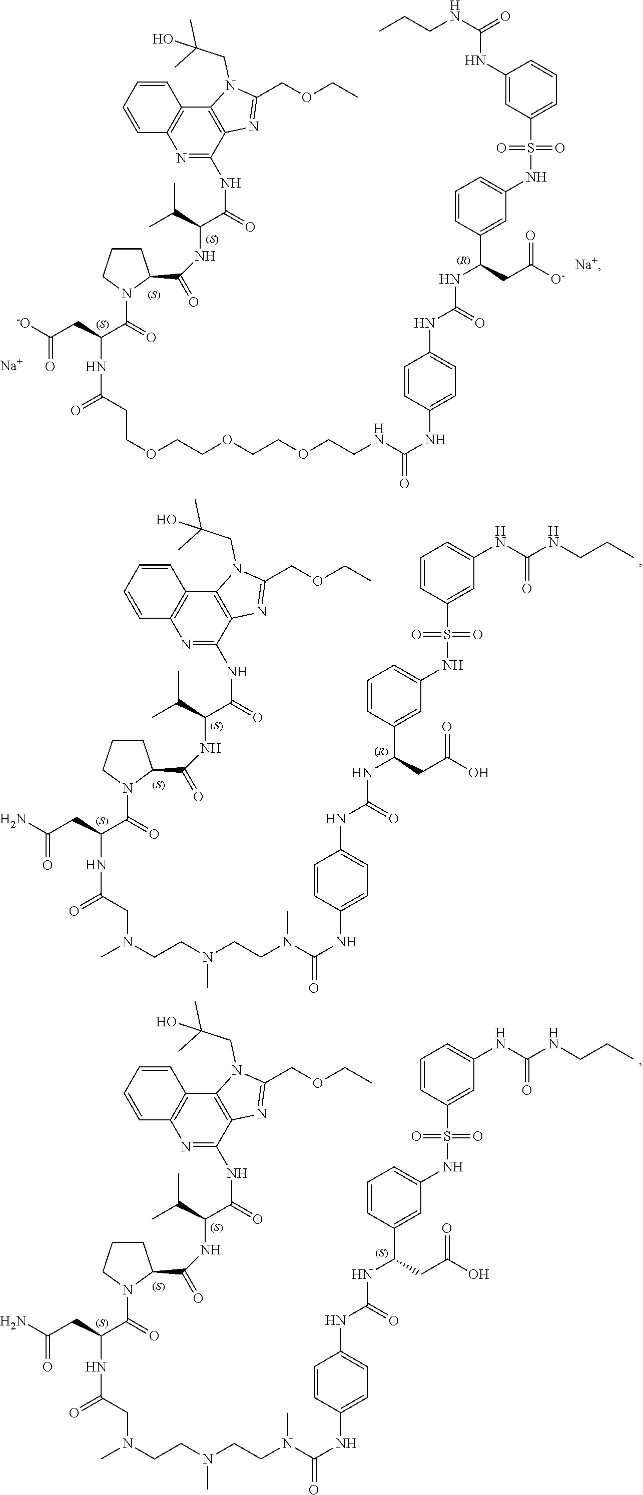

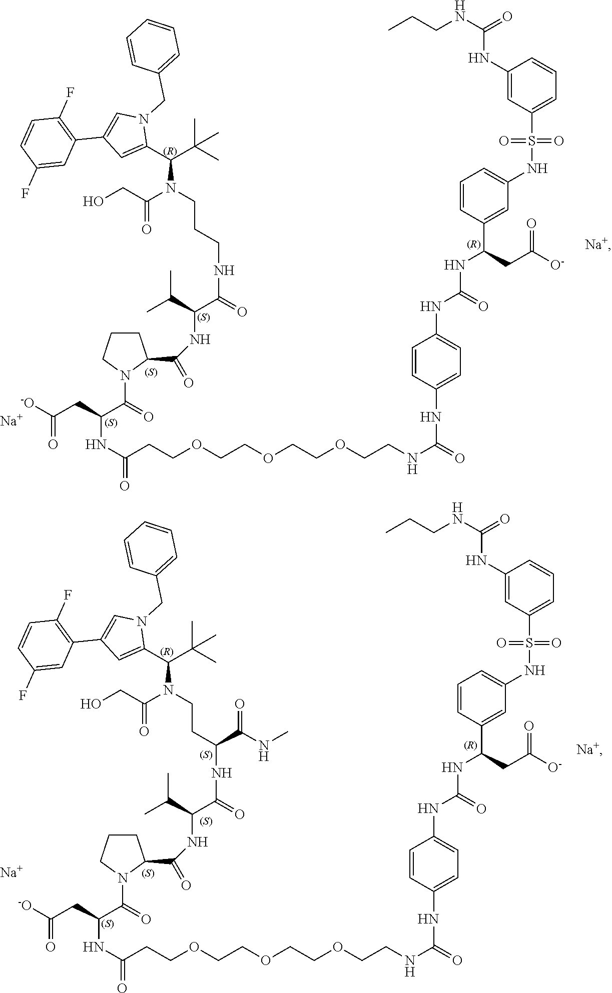

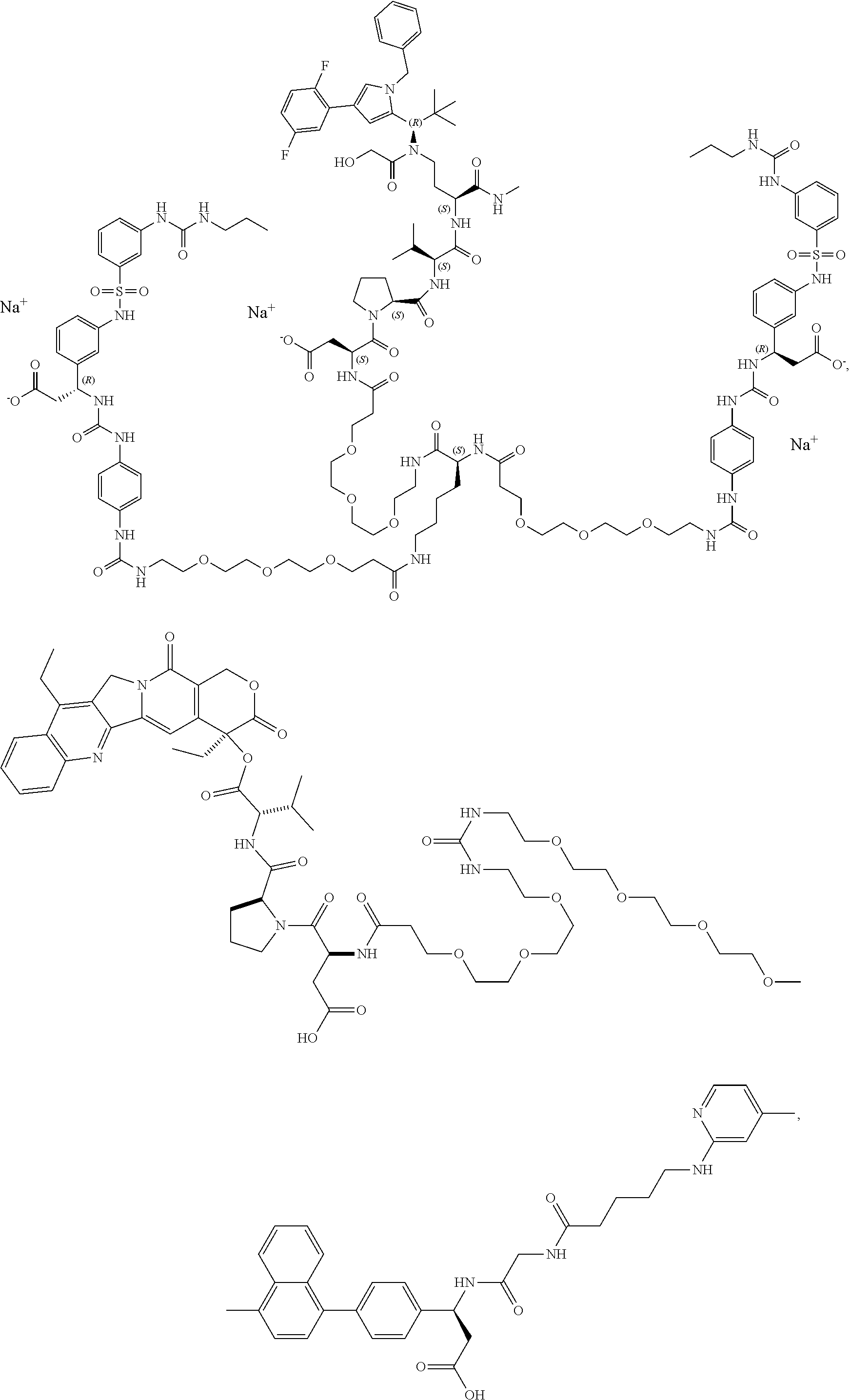

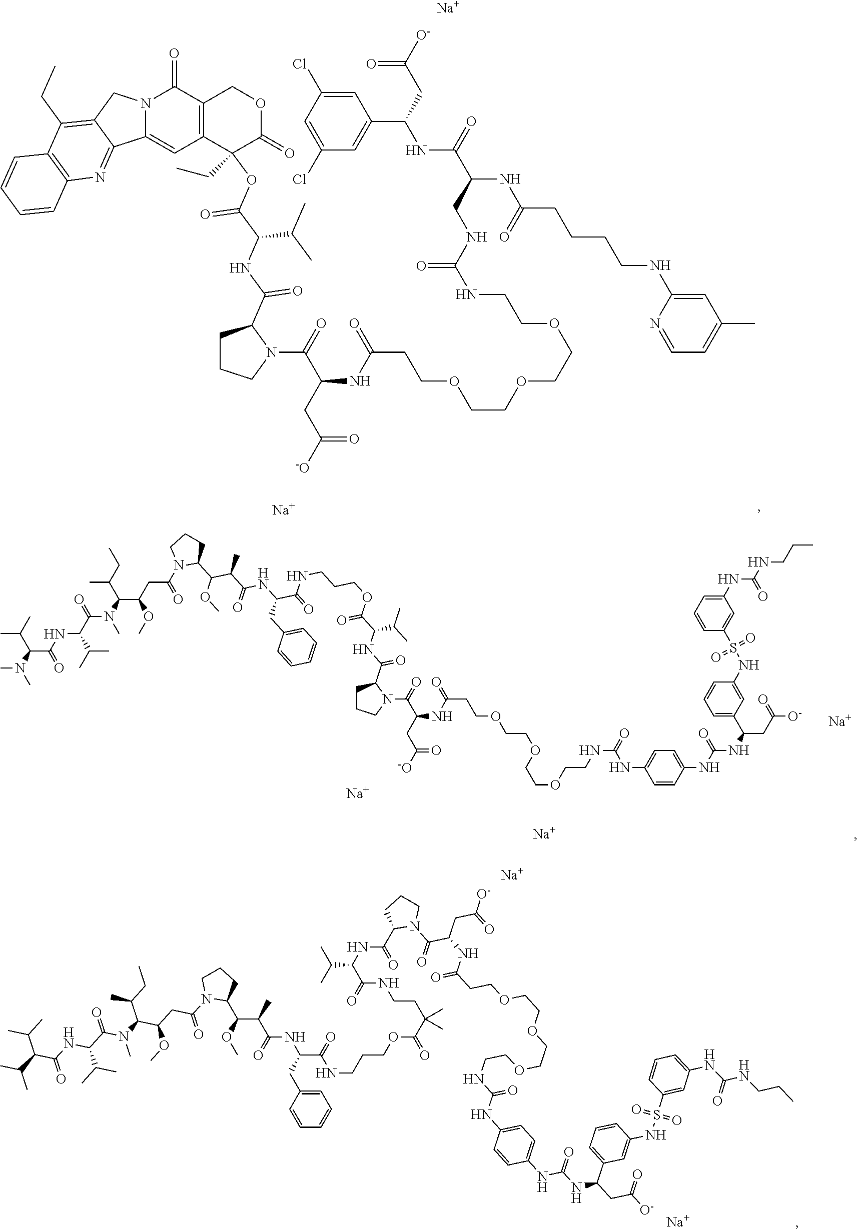

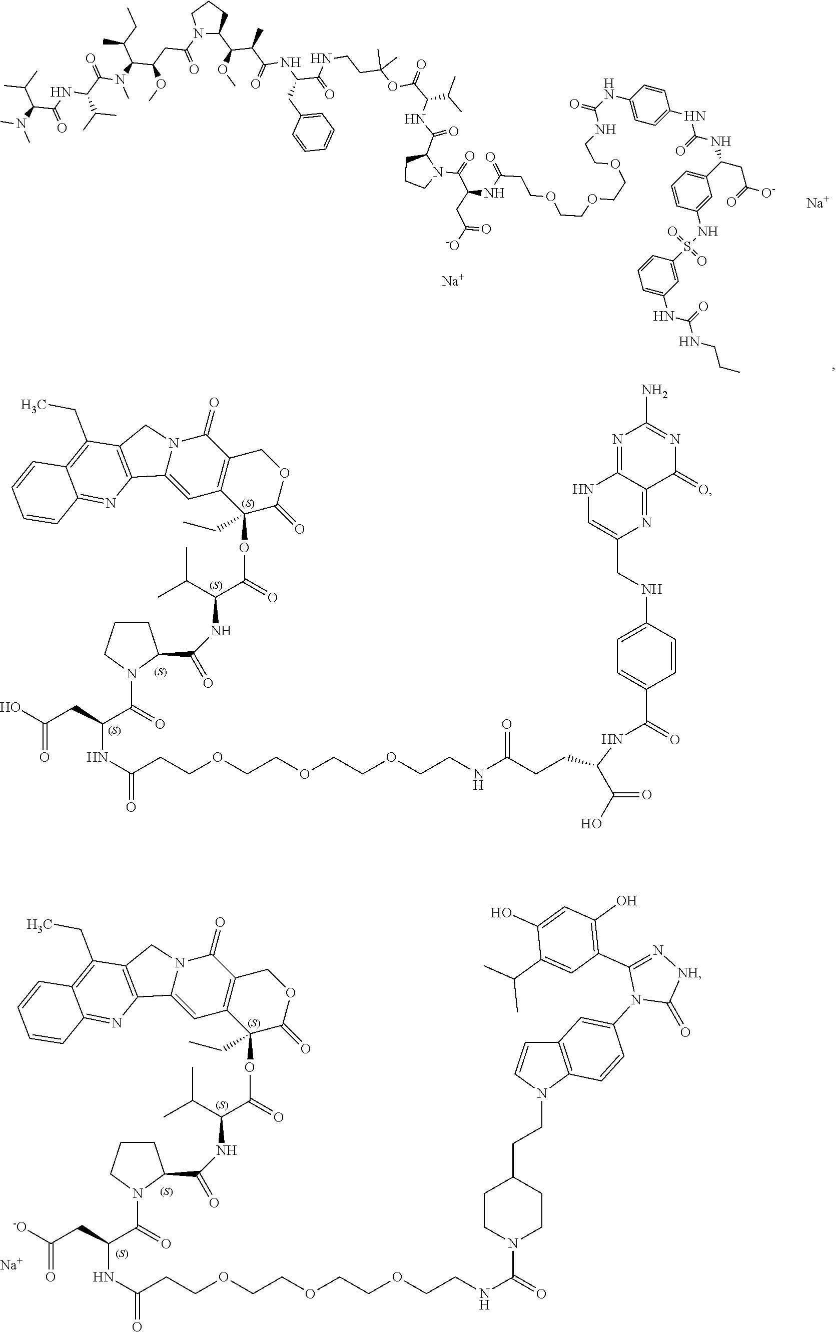

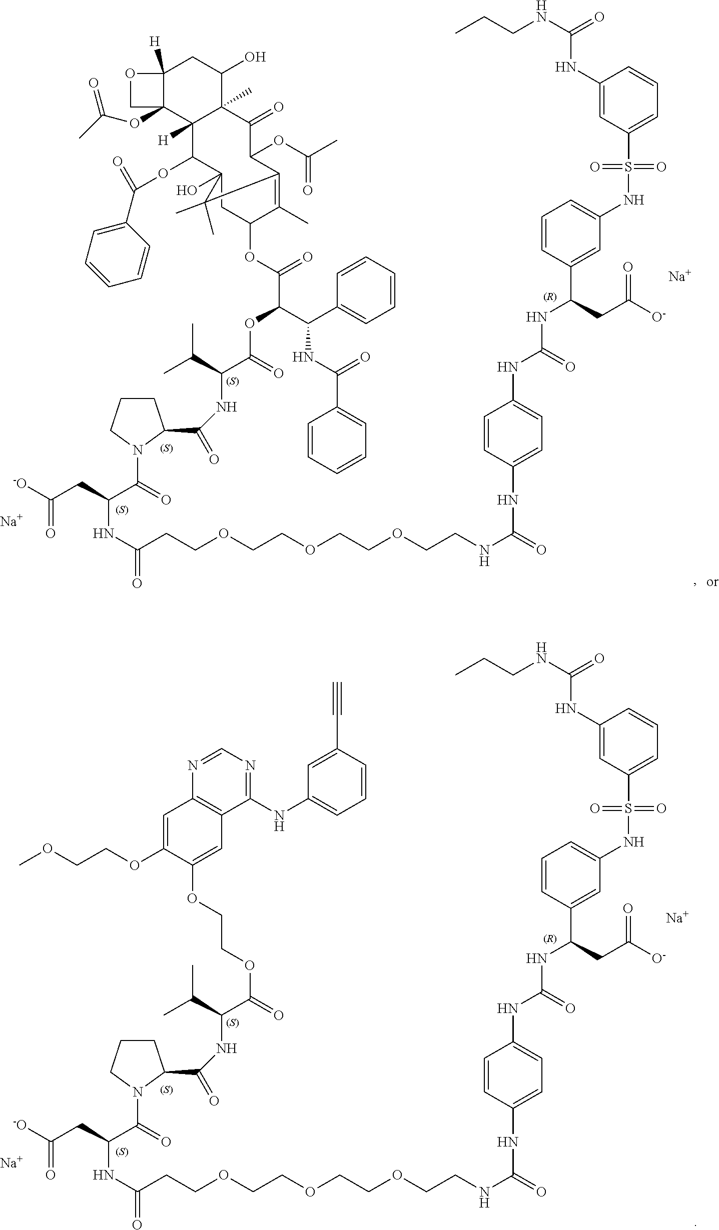

Non-Cleavable Linkers (L¹, L², and/or L³)

Provided herein are compounds having linkers or spacers (used interchangeably) which serve to create physical space between one or more elements of the compound or conjugate. In some embodiments, the linkers or spacers provide additional utility (e.g., functional linkers). For example, in some embodiments, a linker is provided with a particular length that enables enhanced cleavage of an adjacent enzymatically cleavable moiety (e.g., EL). In some embodiments, a linker is provided with a particular length that reduces steric hinderance and/or enables greater target binding. In some embodiments, a linker is provided in a particular length such that an integrin binder can bind an integrin receptor (e.g., an αᵥβ₃ integrin receptor) with potency (i.e., IC₅₀) that is 1.0⁻⁹ M or lower (e.g., 9E⁻¹⁰, 8E⁻¹⁰, 7E⁻¹⁰, 6E⁻¹⁰, 5E⁻¹⁰, 4E⁻¹⁰, 3E⁻¹⁰, 2E⁻¹⁰, 1E⁻¹⁰, or lower). In some embodiments, a linker is provided in a particular length such that an integrin binder can bind an integrin receptor (e.g., an αᵥβ₃ integrin receptor) with potency (i.e., IC₅₀) that is 1·10⁻¹⁰ M or lower (e.g., 9E⁻¹¹, 8E⁻¹¹, 7E⁻¹¹, 6E⁻¹¹, 5E⁻¹¹, 4E⁻¹¹, 3E⁻¹¹, 2E⁻¹¹, 1E⁻¹¹, or lower). In some embodiments, a linker is provided in a particular length such that an integrin binder can bind an integrin receptor (e.g., an αᵥβ₃ integrin receptor) with potency (i.e., IC₅₀) that is 1.0⁻¹¹ M or lower (e.g., 9E⁻¹², 8E⁻¹², 7E⁻¹², 6E⁻¹², 5E⁻¹², 4E⁻¹², 3E⁻¹², 2E⁻¹², 1E⁻¹², or lower).

In some embodiments, provided herein is a compound comprising a linker (e.g., L¹, L², and/or L³), wherein the linker enhances retention of the compound within a tumor microenvironment. In some embodiments, a linker disclosed herein (e.g., a polyamine or polyamide linker) increases the tumor to plasma ratio of a compound, compared to a reference compound comprising an alkyl or PEG linker. In some embodiments, provided herein is a compound comprising a trivalent linker (A¹) and two linker-binder groups (L²IN, L³IN), or a linker-binder and linker-MOD group (L²IN)(L³MOD), wherein the compound comprising the L³IN or L³MOD group has an increased half-life and/or increased AUC, compared to a compound comprising a bivalent linker. In some embodiments, provided herein is a compound comprising a trivalent linker and two integrin binding groups, wherein the compound comprising a trivalent linker and two integrin-bonding groups has an increased half-life and/or increased AUC, compared to a compound comprising a bivalent linker.

In some embodiments, a linker is a branched or linear chain of atoms selected from C, N, O, or S (each of which being substituted with hydrogens or bonds so as to fulfill standard valence). In some embodiments, a linker is a carbonyl (—C(O)—). In some embodiments, a linker contains six or less (non-hydrogen) atoms. In some embodiments, a linker contains about 10 to about 20 (non-hydrogen) atoms. In some embodiments, a linker contains about 10 to about 20 (non-hydrogen) atoms arranged in a linear chain. In some embodiments, a linker contains about 20 to about 30 (non-hydrogen) atoms. In some embodiments, a linker contains about 20 to about 30 (non-hydrogen) atoms arranged in a linear chain. In some embodiments, a linker contains about 30 to about 40 (non-hydrogen) atoms. In some embodiments, a linker contains about 30 to about 40 (non-hydrogen) atoms arranged in a linear chain. In some embodiments, multiple linkers are present, each of which having a different length. In some embodiments, a linker contains cyclic or branched moieties. In some embodiments, a linker is substituted with one or more alkyl, oxo, amino, or amide groups.

In some embodiments, each of L¹, L², and/or L³ contains or is terminally substituted with one or more carbonyl groups (—C(O)—), amine groups (e.g., —NH— or —N(CH₃)—), or amide groups (e.g., —C(O)NH—, —C(O)N(CH₃)—, —NHC(O)—, or N(CH₃)C(O)—).

In some embodiments, each of L¹, L², and/or L³ is a substituted or unsubstituted C₂₋₂₀ alkyl chain that is optionally interrupted one or more times by groups selected from —O—, —NH—, —N(CH₃)—, —C(O)—, —C(O)NH—, —C(O)N(CH₃)—, —C(O)O—, —NHC(O)—, —N(CH₃)C(O)—, —NHC(O)NH—, —S—, —S(O)—, —S(O)₂—, or any combination thereof. In some embodiments, L¹, L², and/or L³ is a substituted or unsubstituted C₂₋₂₀ alkyl chain that is optionally interrupted one or more times by groups selected from —C(O)—, —C(O)NH—, —C(O)N(CH₃)—, —C(O)O—, —NH—, —N(CH₃)—, —NHC(O)—, —N(CH₃)C(O)—, —NHC(O)NH—, —NHS(O)₂NH—, —NHS(O)₂NHC(O)—, —NHS(O)₂NHC(O)O—, —O—, —S—, —S(O)—, —S(O)₂—, —S(O)₂NH—, —S(O)₂NHC(O)—, —S(O)₂NHC(O)NH—, —S(O)₂NHC(O)O—, carbocyclyl, heterocyclyl, aralkyl, heteroaralkyl, or any combination thereof. In some embodiments, L¹, L², and/or L³ is a linker disclosed in WO2016207089, which is incorporated by reference in its entirety. In some embodiments, a linker comprises a C₁₋₃₀ alkyl or heteroalkyl group (optionally substituted), which is optionally interrupted by an aryl or heteroaryl group (e.g., a triazole, a dibenzocyclooctyne, or a derivative thereof). For example, a linker may comprise a dibenzylcyclooctyne (DBCO) derivative such as a DBCO NHS ester, or a chemical group formed therefrom. For example, an interrupting group may include:

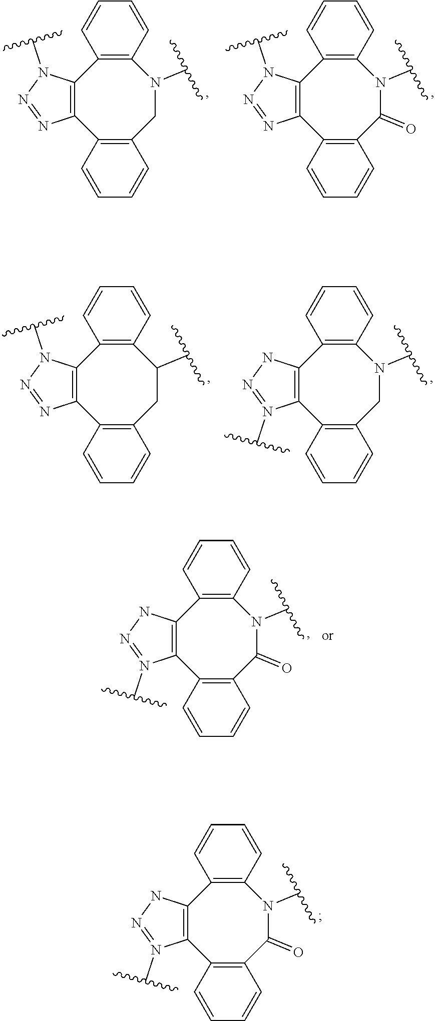

or a derivative thereof, wherein the interrupting group is substituted on each end to form a linker (e.g., L¹, L², and/or L³). In some embodiments, an interrupting group may include: —NHS(O)₂NH—, —NHS(O)₂NHC(O)—, —NHS(O)₂NHC(O)O—, or

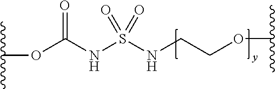

(wherein y is 1-20), or any combination thereof. In some embodiments, L¹, L², and/or L³ is a sulfamide linker, comprising one or more (e.g., one, two, three, or four) simple spacers defined herein, optionally interrupted (i.e., conjoined) by a sulfamide group (e.g., —NHS(O)₂NH—, —NHS(O)₂NHC(O)—, or —NHS(O)₂NHC(O)O—). In some embodiments, L¹, L², and/or L³ comprises a sulfamide group (—NHS(O)₂NH—). In some embodiments, L¹, L², and/or L³ is a sulfamide linker, consisting of one or more (e.g., one, two, three, or four) of the following groups: —NHS(O)₂NH—, —NHS(O)₂NHC(O)—, —NHS(O)₂NHC(O)O—, and

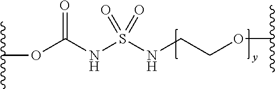

(wherein y is 1-20), and wherein the sulfamide linker is optionally conjoined to an adjacent group via a spanner (as defined herein).

In some embodiments, a linker is ionized in a biological system (e.g., within an acidic tumor microenvironment). In some embodiments, an ionized or partially charged (e.g., partially positive or partially negative) moiety or moieties within a linker enhance localization of the compound in a preferred locale (e.g., extracellularly, within a tumor microenvironment).

In some embodiments, a linker (e.g., one or more of L¹, L², and/or L³) is an optionally substituted polyamine linker or an optionally substituted polyamide linker.

In some embodiments, a polyamine or polyamide linker is a functional linker. In some embodiments, the functional linker is a polyamide or a sulfamide linker, wherein the functional linker increases retention of the compound within a tumor microenvironment (i.e., relative to an alkyl or PEG linker) and/or decreases liver exposure. In some embodiments, a functional linker (e.g., a polyamide linker) reduces immunogenic response to a compound containing said functional linker (i.e., relative to a PEG linker). In some embodiments, a polyamide or sulfamide linker disclosed herein improves one or more pharmacokinetic parameters. In some embodiments, a polyamide or sulfamide linker disclosed herein reduces liver toxicity, increase tumor-to-liver exposure, and/or increases liver clearance of a compound containing said functional linker. In some embodiments, a linker disclosed herein increases stability (i.e., reduces off-target release of a pa In some embodiments, biodistribution, AUC, and/or solubility are impacted (e.g., favorably) by a functional linker disclosed herein.

In some embodiments, the polyamine or polyamide linker of one or more of L¹, L², and/or L³ is substituted with one or more carbonyl groups. In some embodiments, the polyamine linker of one or more of L¹, L², and/or L³ forms an aminium ion (or optionally multiple aminium ions) in an acidic tumor microenvironment. In some embodiments, the polyamine or polyamide linker of one or more of L¹, L², and/or L³ is selectively retained in a tumor microenvironment.

In some embodiments, the present invention provides compounds having a structure of formulae disclosed herein, or a pharmaceutically acceptable salt thereof, or a stereoisomer or mixture of stereoisomers thereof, wherein each of L¹, L², and/or L³ is independently a bond, substituted or unsubstituted C₁₋₃₀ alkyl, or substituted or unsubstituted heteroalkyl. In some embodiments, each of L¹, L², and/or L³ is independently —C(O)—, substituted or unsubstituted C₂₋₃₀ alkyl, or substituted or unsubstituted heteroalkyl.

In some embodiments, each of L¹, L², and/or L³ is a substituted or unsubstituted C₂₋₂₀ alkyl chain that is optionally interrupted one or more times by groups, each independently selected from —O—, —S—, —NH—, —N(CH₃)—, —C(O)—, —C(O)NH—, —C(O)N(CH₃)—, —C(O)O—, —NHC(O)—, N(CH₃)C(O)—, or —NHC(O)NH—, or any combination thereof.

In some embodiments, each of L¹, L², and/or L³, contains (e.g., is terminally substituted with) one or more carbonyl groups (—C(O)—), amine groups (e.g., —NH— or —N(CH₃)—), or amide groups (e.g., —C(O)NH—, —C(O)N(CH₃)—, —NHC(O)—, or N(CH₃)C(O)—). In some embodiments, spacers such as L¹, L², and/or L³ contain one or more polymeric units selected from the group consisting of:

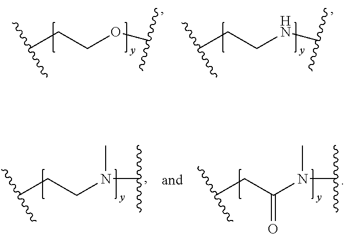

In some embodiments, the spacer comprises one to twenty polymeric units (i.e., y is 1-20). In some embodiments, y is between 2 and 6 (e.g., y is 2 to 6, 2 to 4, 2 or 3, 3 to 6, 3 or 4, etc.). In some embodiments, y is between 6 and 12 (e.g., y is 6 to 12, 6 to 10, 6 to 8, 8 to 12, 8 to 10, etc.). In some embodiments, y is 2, 3, or 4. In some embodiments, y is 8, 9, or 10. In some embodiments, one or more of L¹, L², and/or L³ is an optionally substituted polyamine linker or an optionally substituted polyamide linker.

In some embodiments, L¹, L², and/or L³ is a simple spacer selected from the group consisting of:

| | |
|---|---|
| —O— | —S— |
| —S(O)₂— | —C(O)— |
| —C₁₋₃₀ alkyl-, | —C(O)—C₁₋₃₀ alkyl |
| —C₁₋₃₀ alkyl-C(O)—, | —C(O)—C₁₋₃₀ alkyl-C(O)—, |
| —C₁₋₃₀ alkyl-C(O)NH—, | —C(O)—C₁₋₃₀ alkyl-C(O)NH—, |
| —C₁₋₃₀ alkyl-C(O)N(CH₃)—, | —C(O)—C₁₋₃₀ alkyl-C(O)N(CH₃)—, |
| —C₁₋₃₀ alkyl-NH—, | —C(O)—C₁₋₃₀ alkyl-NH—, |
| —C₁₋₃₀ alkyl-NHC(O)—, | —C(O)—C₁₋₃₀ alkyl-NHC(O)—, |
| —C₁₋₃₀ alkyl-N(CH₃)—, | —C(O)—C₁₋₃₀ alkyl-N(CH₃)—, |
| —C₁₋₃₀ alkyl-N(CH₃)C(O)—, | —C(O)—C₁₋₃₀ alkyl-N(CH₃)C(O)—, |
| —C(O)NH—, | —C(O)N(CH₃)— |
| —C(O)NH—C₁₋₃₀ alkyl-, | —C(O)N(CH₃)—C₁₋₃₀ alkyl |
| —C(O)NH—C₁₋₃₀ alkyl-C(O)—, | —C(O)N(CH₃)—C₁₋₃₀ alkyl-C(O)—, |
| —C(O)NH—C₁₋₃₀ alkyl-C(O)NH—, | —C(O)N(CH₃)—C₁₋₃₀ alkyl-C(O)NH—, |
| —C(O)NH—C₁₋₃₀ alkyl-C(O)N(CH₃)—, | —C(O)N(CH₃)—C₁₋₃₀ alkyl-C(O)N(CH₃)—, |
| —C(O)NH—C₁₋₃₀ alkyl-NH—, | —C(O)N(CH₃)—C₁₋₃₀ alkyl-NH—, |
| —C(O)NH—C₁₋₃₀ alkyl-NHC(O)—, | —C(O)N(CH₃)—C₁₋₃₀ alkyl-NHC(O)—, |
| —C(O)NH—C₁₋₃₀ alkyl-N(CH₃)—, | —C(O)N(CH₃)—C₁₋₃₀ alkyl-N(CH₃)—, |
| —C(O)NH—C₁₋₃₀ alkyl-N(CH₃)C(O)—, | —C(O)N(CH₃)—C₁₋₃₀ alkyl-N(CH₃)C(O)—, |
| —NH—, | —NHC(O)—, |
| —NH—C₁₋₃₀ alkyl-, | —NHC(O)—C₁₋₃₀ alkyl-, |
| —NH—C₁₋₃₀ alkyl-C(O)—, | —NHC(O)—C₁₋₃₀ alkyl-C(O)—, |
| —NH—C₁₋₃₀ alkyl-C(O)NH—, | —NHC(O)—C₁₋₃₀ alkyl-C(O)NH—, |
| —NH—C₁₋₃₀ alkyl-C(O)N(CH₃)—, | —NHC(O)—C₁₋₃₀ alkyl-C(O)N(CH₃)—, |
| —NH—C₁₋₃₀ alkyl-NH—, | —NHC(O)—C₁₋₃₀ alkyl-NH—, |
| —NH—C₁₋₃₀ alkyl-NHC(O)—, | —NHC(O)—C₁₋₃₀ alkyl-NHC(O)—, |
| —NH—C₁₋₃₀ alkyl-N(CH₃)—, | —NHC(O)—C₁₋₃₀ alkyl-N(CH₃)—, |
| —NH—C₁₋₃₀ alkyl-N(CH₃)C(O)—, | —NHC(O)—C₁₋₃₀ alkyl-N(CH₃C(O)—, |
| —N(CH₃)—, | —N(CH₃)C(O)—, |
| —N(CH₃)—C₁₋₃₀ alkyl-, | —N(CH₃)C(O)—C₁₋₃₀ alkyl-, |
| —N(CH₃)—C₁₋₃₀ alkyl-C(O)—, | —N(CH₃)C(O)—C₁₋₃₀ alkyl-C(O)—, |
| —N(CH₃)—C₁₋₃₀ alkyl-C(O)NH—, | —N(CH₃)C(O)—C₁₋₃₀ alkyl-C(O)NH—, |
| —N(CH₃)—C₁₋₃₀ alkyl-C(O)N(CH₃)—, | —N(CH₃)C(O)—C₁₋₃₀ alkyl-C(O)N(CH₃)—, |
| —N(CH₃)—C₁₋₃₀ alkyl-NH—, | —N(CH₃)C(O)—C₁₋₃₀ alkyl-NH—, |
| —N(CH₃)—C₁₋₃₀ alkyl-NHC(O)—, | —N(CH₃)C(O)—C₁₋₃₀ alkyl-NHC(O)—, |
| —N(CH₃)—C₁₋₃₀ alkyl-N(CH₃)—, | —N(CH₃)C(O)—C₁₋₃₀ alkyl-N(CH₃)—, |
| —N(CH₃)—C₁₋₃₀ alkyl-N(CH₃)C(O)—, and | —N(CH₃)C(O)—C₁₋₃₀ alkyl-N(CH₃C(O)—. |

In some embodiments, L¹, L², and/or L³ is a compound linker comprising two or more (e.g., one, two, three, or four) simple spacer elements defined above. In some embodiments, the two or more elements of the compound linker are conjoined by an interrupting group (e.g, a sulfamide group (e.g., —NHS(O)₂NH—, —NHS(O)₂NHC(O)—, or —NHS(O)₂NHC(O)O—), or a heteroaryl or heteroaralkyl (e.g., a substituted triazole, a substituted DBCO, or a combination or derivative thereof). In some embodiments, the compound linker further comprises one or more units selected from the group consisting of

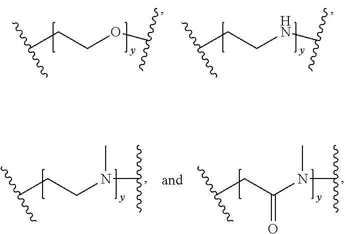

(e.g., wherein y is I to 20).

In some embodiments, L¹, L², and/or L³ is a combination of two or three simple spacers. In some embodiments, L¹, L², and/or L³ is a combination of two or three simple spacers, interrupted by (e.g., conjoined by) an amino acid, a dipeptide, or a tripeptide. In some embodiments, L¹, L², and/or L³ is a combination of two or three simple spacers, interrupted by (e.g., conjoined by) a cycloalkyl, heterocycloalkyl, aryl, or heteroaryl. In some embodiments, the interrupting group is a substituted triazole. In some embodiments, the interrupting group is an amino acid. In some embodiments, the interrupting group is: —S—, —S(O)—, —S(O)₂—, —NHS(O)₂NH—, —NHS(O)₂NHC(O)—, —NHS(O)₂NHC(O)O—,

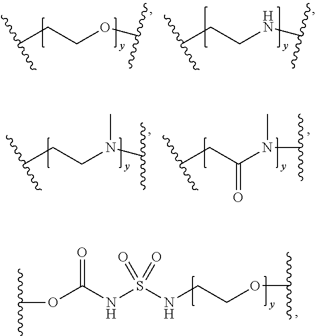

(wherein y is 1-20),

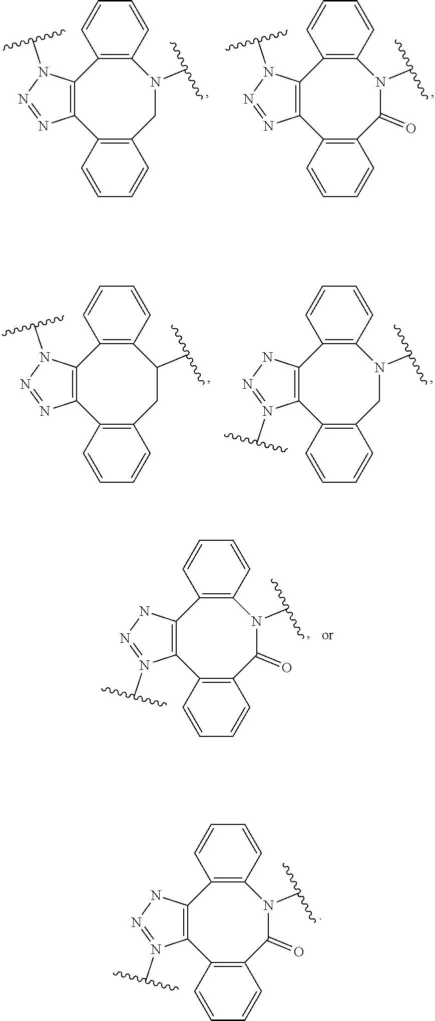

In some embodiments, L¹, L², and/or L³ is a substituted or unsubstituted C₂₋₂₀ alkyl chain that is optionally interrupted one or more times by groups selected from —C(O)—, —C(O)NH—, —C(O)N(CH₃)—, —C(O)O—, —NH—, —N(CH₃)—, —NHC(O)—, —N(CH₃)C(O)—, —NHC(O)NH—, —O—, —S—, —S(O)—, —S(O)₂—, carbocyclyl, heterocyclyl, aralkyl, heteroaralkyl, or any combination thereof. In some embodiments, L¹, L², and/or L³ is a linker disclosed in WO2016207089, which is incorporated by reference in its entirety. In some embodiments, a linker comprises two simple spacers which are conjoined via an aryl or heteroaryl group (e.g., a triazole, a dibenzocyclooctyne, or a derivative thereof). For example, a linker may comprise a dibenzylcyclooctyne (DBCO) derivative such as a DBCO NHS ester, or a chemical group formed therefrom. For example, an interrupting group may include:

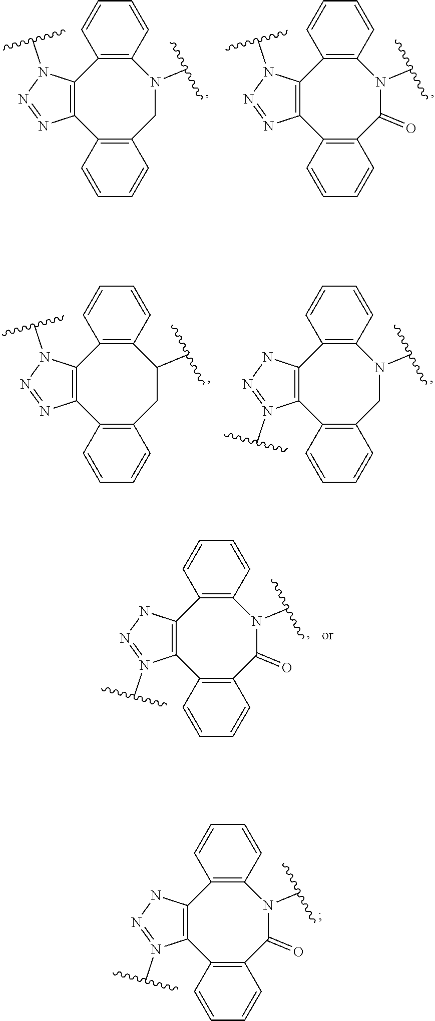

or a derivative thereof, wherein the interrupting group is substituted on each end to form a linker.

In some embodiments, L¹, L², and/or L³ is a linker having a structure represented by formula (i), (ii), (iii), (iv), or (v) below:

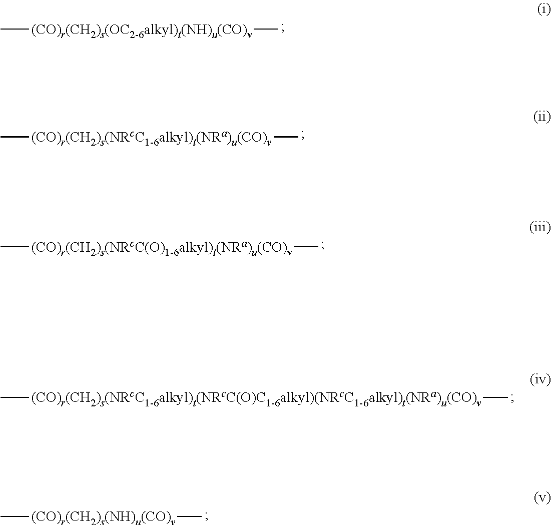

wherein:
R^{a} is, in each instance, independently selected from hydrogen or C₁₋₃ alkyl;
R^{c} is, in each instance, independently selected from hydrogen or C₁₋₃ alkyl;
r is 0 or 1; s is 0 to 10; t is 1 to 10; u is 0 or 1; and v is 0 or 1.

In some embodiments, L¹, L², and/or L³ is a polymeric linker selected from:

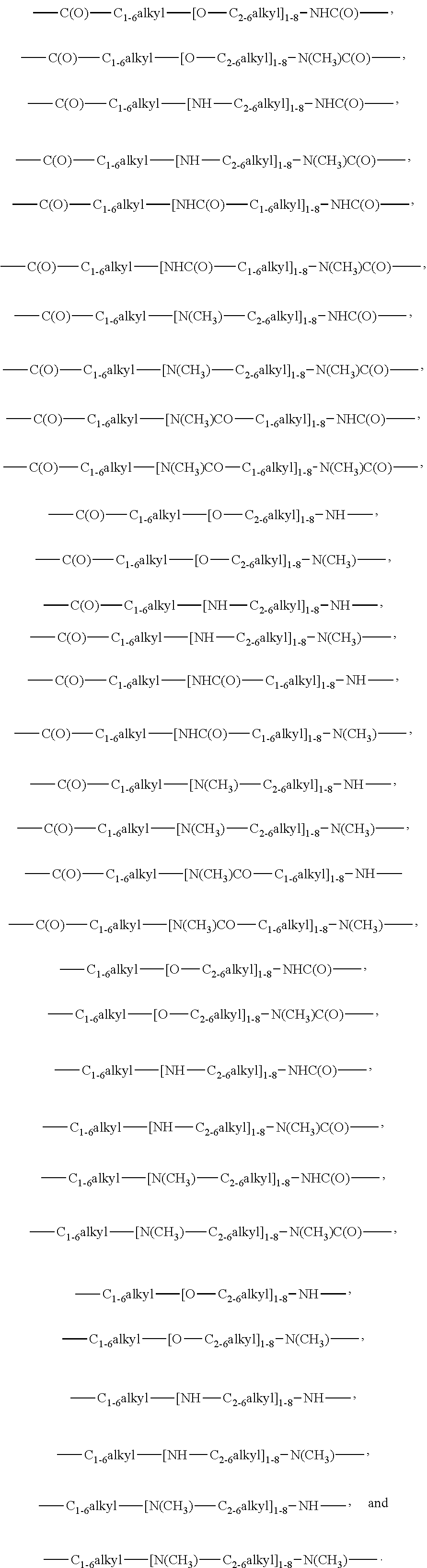

In some embodiments, L¹ is selected from:

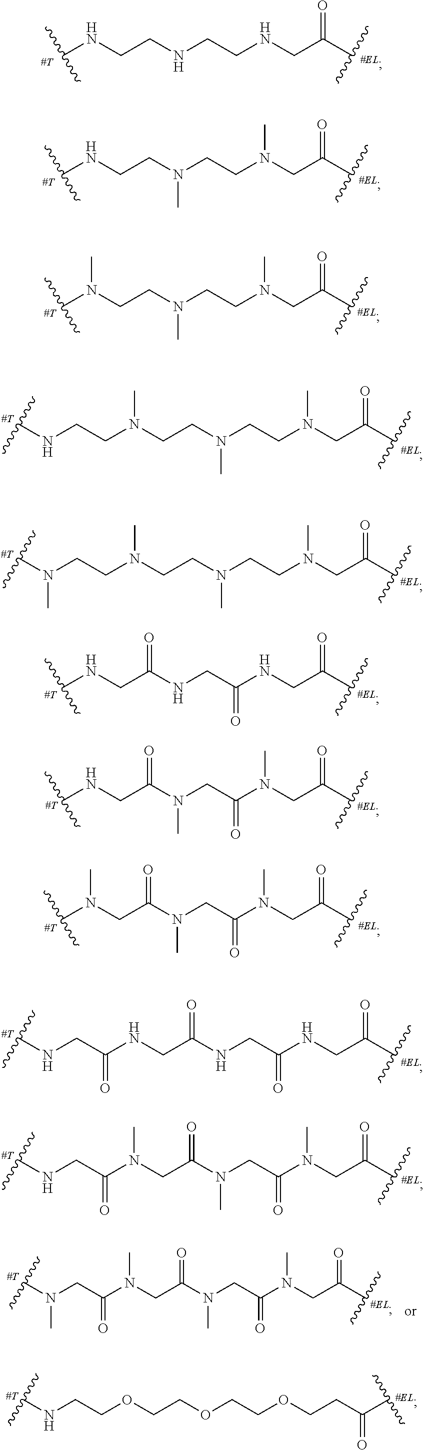

Wherein:
#EL is a bond to the peptide linker (EL); and
#T is a bond to a target protein binder (T).

In some embodiments, L² is selected from:

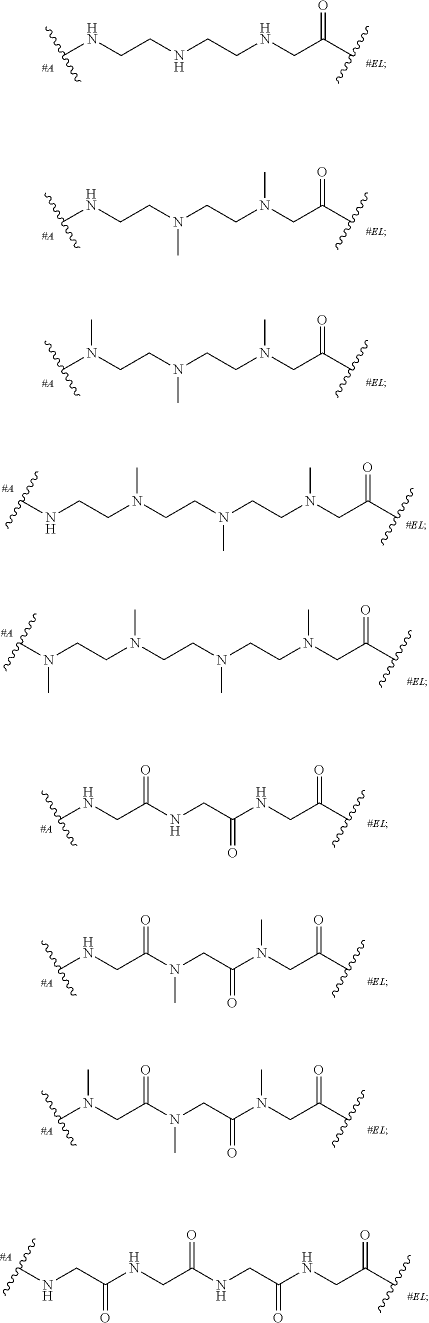

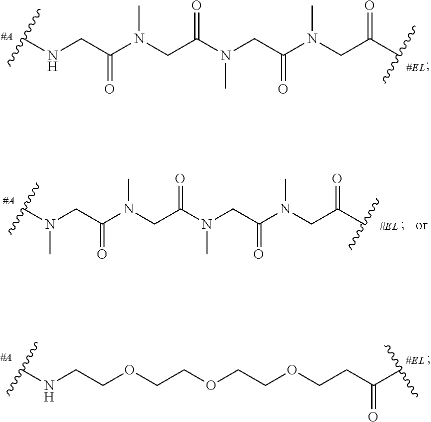 [SMILES: *NCC(=O)N(C)CC(=O)N(C)CC(=O)N(C)CC(*)=O ; *C(=O)CN(C)C(=O)CN(C)C(=O)CN(C)C(=O)CN(*)C ; *NCCOCCOCCOCCC(*)=O]

wherein:
#EL is a bond to EL; and
#A is a bond to a trivalent or tetravalent linker A (i.e., A¹ or A² respectively).

In some embodiments, L³ is selected from:

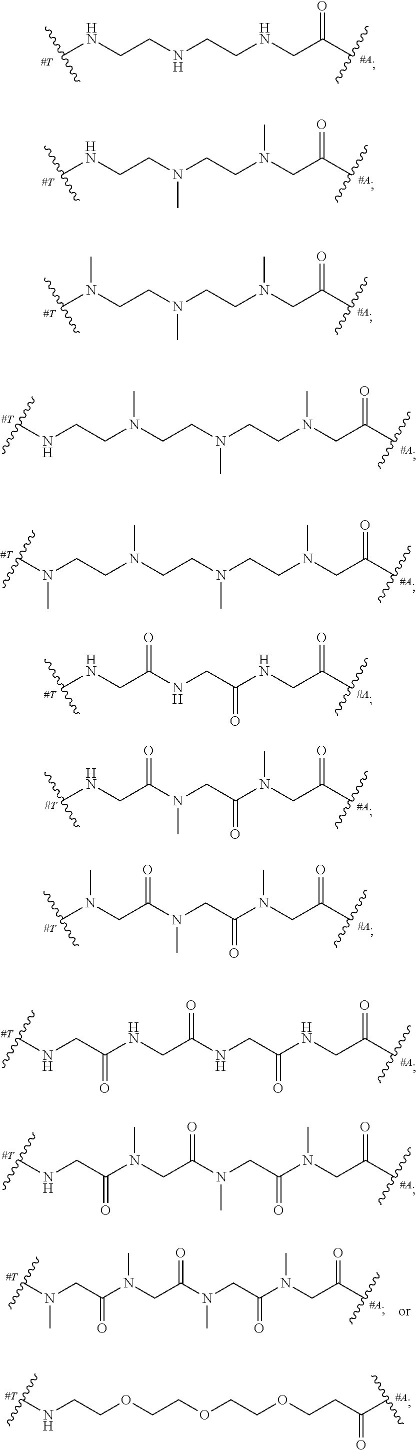

Wherein:
#A is a bond to a trivalent or tetravalent linker A (i.e., A¹ or A² respectively); and
#T is a bond to a target protein binder (T).

### Self-Immolative Linkers (SIL)

To assure efficient release of the free drug, it is optionally also possible to incorporate what are called self-immolative linker elements (SIL) between the enzymatic cleavage site and drug (Anticancer Agents in Medicinal Chemistry, 2008, 8, 618-637). The drug can be released by various mechanisms, for example after initial enzymatic release of a nucleophilic group by subsequent elimination via an electronic cascade (Bioorg. Med. Chem., 1999, 7, 1597; J. Med. Chem., 2002, 45, 937; Bioorg. Med. Chem., 2002, 10, 71) or by cyclization of the corresponding linker element (Bioorg. Med. Chem., 2003, 11, 2277; Bioorg. Med. Chem., 2007, 15, 4973; Bioorg. Med. Chem. Lett., 2007, 17, 2241, Org. Biomol. Chem., 2011, 9, 1846-1854) or by a combination of the two (Angew. Chem. Inter. Ed., 2005, 44, 4378). Examples of such linker elements are shown in Scheme 1:

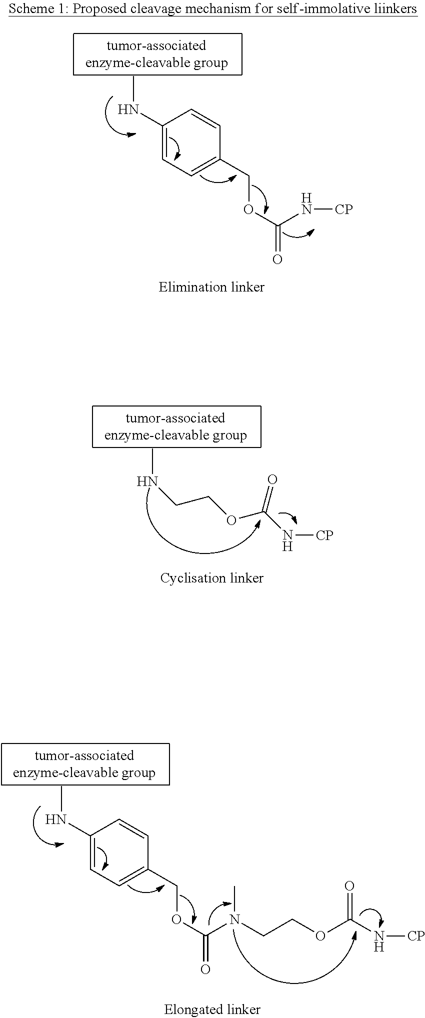

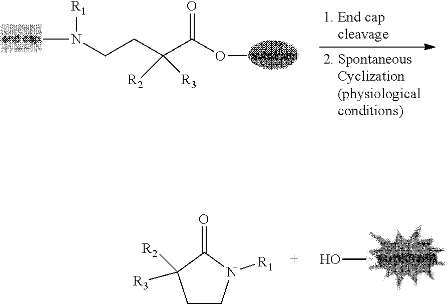

In some embodiments, SIL is a para-amino carbamate (PABC) group. In some embodiments, SIL is:

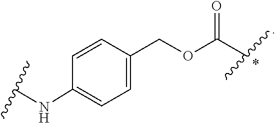 [SMILES: *Nc1ccc(COC(*)=O)cc1]

In some embodiments, SIL is absent (i.e., SIL is a bond). In some embodiments, SIL is

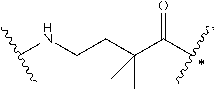 [SMILES: *NCCC(C)(C)C(*)=O]

wherein * denotes a bond to a nitrogen of the payload, and the unmarked radical forms a bond to the enzymatically cleavable peptide.

### Trivalent Linkers (A¹)

In some embodiments, a trivalent linker A¹ is a moiety containing 1 to 100 atoms selected from H, C, N, O, and S, configured to bond to linkers L² and L³. In some embodiments, A¹ contains a central atom or group (Y) that is N or CH. In some embodiments, the central atom or group (Y) has one or more arms (e.g., alkyl or heteroalkyl chains, optionally substituted with, or interrupted by, one or more groups independently selected from carbonyls (—C(O)—), ethers (—O—), amines (e.g., —NH— or —N(CH₃)—), or amides (e.g., —C(O)NH—, —C(O)N(CH₃)—, —NHC(O)—, or —N(CH₃)C(O)—, alternatively referred to as amide linkers). In some embodiments, A¹ is trivalent heteroalkyl radical. In some embodiments, A¹ is trivalent linker with amide linker arms (e.g., B¹, B², B³). In some embodiments, A¹ is a trivalent heteroaryl, heteroaralkyl, aryl, aralkyl, heteroalkyl-aryl, heteroalkyl-heteroaryl radical. In some embodiments, A¹ contains a central atom or group (Y) that is a carbocycle (e.g., cycloalkyl or aryl) or heterocycle (e.g., heterocycloalkyl or heteroaryl). In some embodiments, (Y) is phenyl, triazinyl, or triazolyl, each of which is optionally substituted by 1-3 heteroalkyl arms. In some embodiments, a linker (e.g., L2 or L³) is or comprises an alkyl, alkenyl, alkynyl, heteroalkyl, cycloalkyl, aryl, heterocyclyl, heteroaryl, aralkyl, heteroaralkyl, alkyl-heterocyclyl, heteroalkyl-heterocyclyl, alkyl-aralkyl, heteroalkyl-aralkyl, alkyl-heteroaralkyl, heteroalkyl-heteroaralkyl, alkyl-heterocycloalkyl, or any combination thereof. In some embodiments, a linker (e.g., L¹, L², or L³) is a heteroalkyl or alkyl linker, optionally interrupted with an aryl, heteroaryl, cycloalkyl, or heterocyclyl group. In some embodiments, the linker is interrupted with a click group (e.g., a substituted triazole).

In some embodiments, A or A¹ is a trivalent linker comprising arms B¹, B², and B³, and optionally further comprising spanners S¹, S², and S³, wherein the trivalent linker is configured to form an amide bond with an adjacent group (e.g., L² or L³). In some embodiments, A¹ is a trivalent amide linker. In this context, a trivalent amide linker can be a chemical group having three valencies (or capable of forming bonds to 3 agents), and generally composed of alkyl, carbonyl, and amine groups, and preferably capable of forming an amide bond with an adjacent group (e.g., L², L³). In some embodiments, the trivalent amide linker comprises a central amino acid (e.g., lysine, glutamine, glutamate, etc.), wherein the amino acid forms a bond with an adjacent group (e.g., L², L³), optionally bridged via a linking arm (e.g., B¹, B², B³) and/or a spanner (i.e., S¹, S², or S³). Preferably, a trivalent radical as described herein conjoins a payload (e.g., a protease-cleavable payload (P)) with two target protein binders, or with an target protein binder and a group MOD, and wherein the payload, target protein binder(s) and MOD may be conjoined via a linker (L² or L³).

In some embodiments, one or more of S¹, S², and S³ is a bond. In some embodiments, one or more of S¹, S², and S³ is —C(O)—C₂₋₆ alkyl-C(O)—, —N(CH₃)—C₁₋₆ alkyl-N(CH₃)—, —NH—C₁₋₆ alkyl-NH—, or —NH—C₁₋₆ alkyl-N(CH₃)—. In some embodiments, S¹, S², and/or S³ is —C(O)—C24 alkyl-C(O)— or —NH—C₁₋₆ alkyl-NH—.

In some embodiments, each B¹, B², and B³, is an amide linker (e.g., an alkyl amide and/or a polyamide linker). In some embodiments, each B¹, B², and B³, is an amide linker comprising 1 to about 32 atoms. In some embodiments, the 1 to 32 atoms are selected from carbon, nitrogen, and oxygen.

In some embodiments, each B¹, B², and B³, is selected from: —C(O)—;

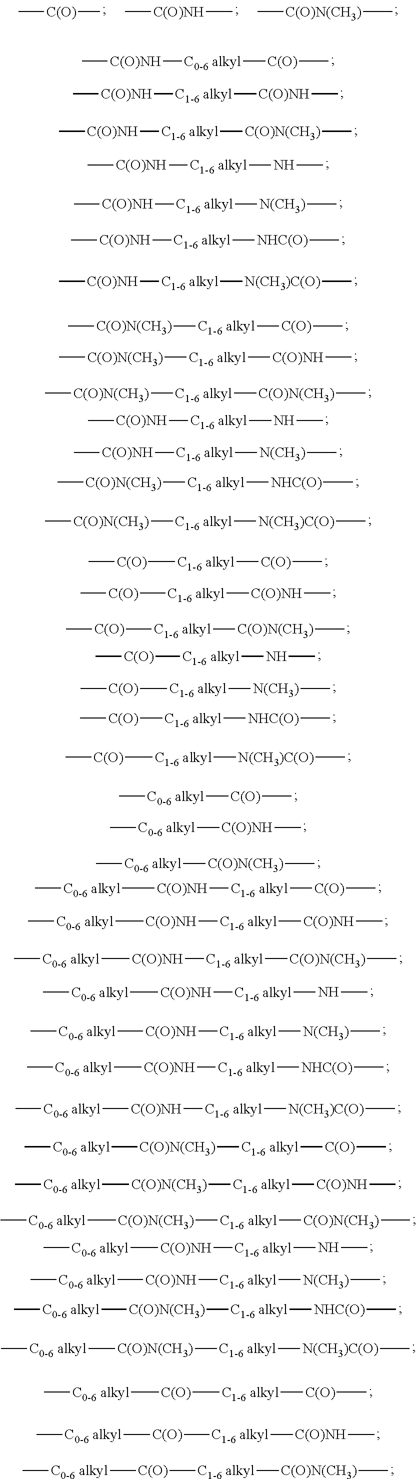

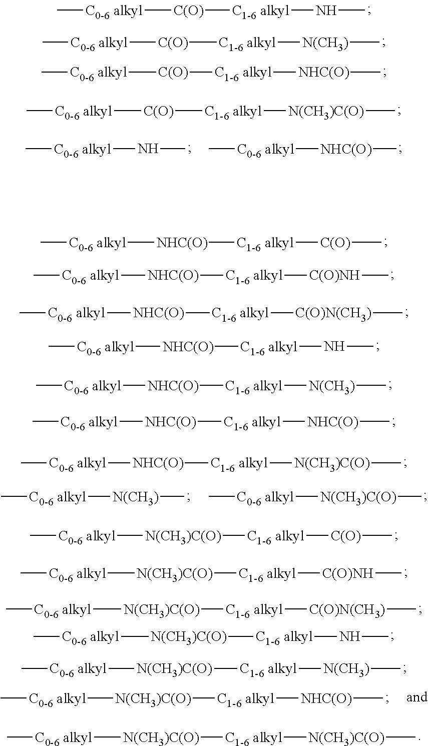

In some embodiments, A¹ is an amino acid or a derivative thereof. In some embodiments, A¹ is a Lys (e.g., L-Lys or D-Lys), Glu (L-Glu or D-Glu), or Asp (L-Asp or D-Asp), or a derivative thereof (e.g., substituted with one or more arms (e.g., B¹, B², or B³)).

In some embodiments, A¹ has one of the following structures:

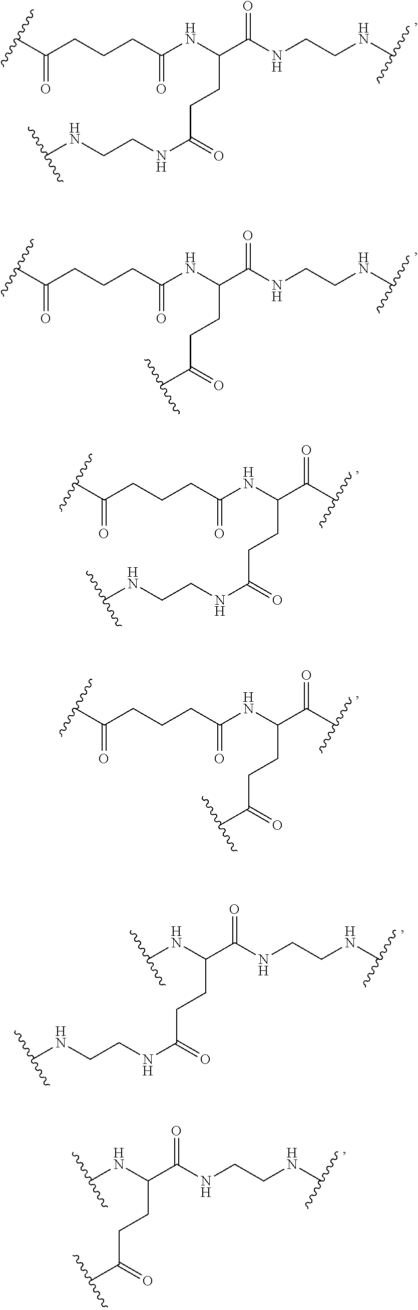 [SMILES: *NCCNC(=O)CCC(NC(=O)CCCC(*)=O)C(=O)NCCN* ; *NCCNC(=O)C(CCC(*)=O)NC(=O)CCCC(*)=O ; *NCCNC(=O)CCC(NC(=O)CCCC(*)=O)C(*)=O ; *C(=O)CCCC(=O)NC(CCC(*)=O)C(*)=O ; *NCCNC(=O)CCC(N*)C(=O)NCCN* ; *NCCNC(=O)C(CCC(*)=O)N*]

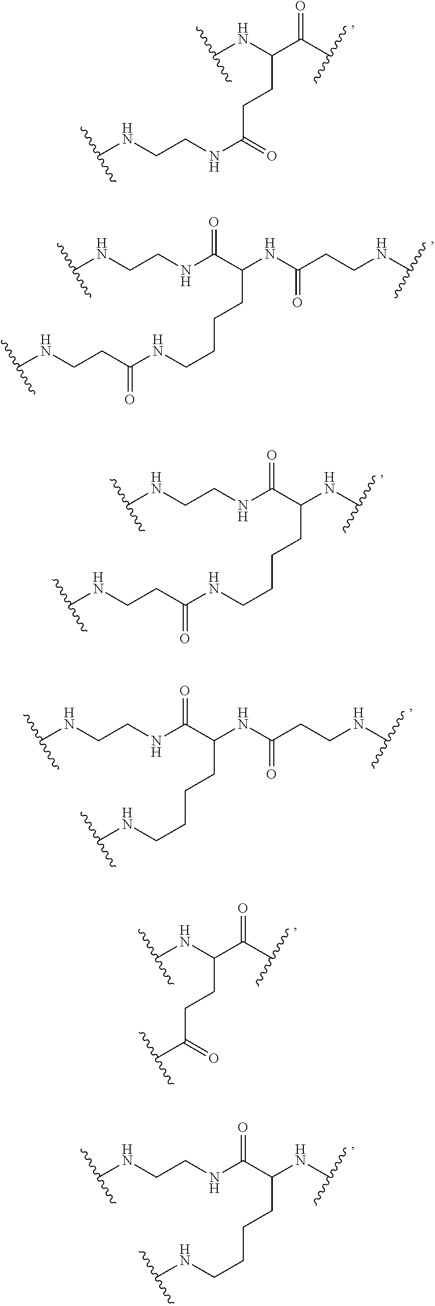 [SMILES: *NCCNC(=O)CCC(N*)C(*)=O ; *NCCNC(=O)C(CCCCNC(=O)CCN*)NC(=O)CCN* ; *NCCNC(=O)C(CCCCNC(=O)CCN*)N* ; *NCCCCC(NC(=O)CCN*)C(=O)NCCN* ; *NC(CCC(*)=O)C(*)=O ; *NCCCCC(N*)C(=O)NCCN*]

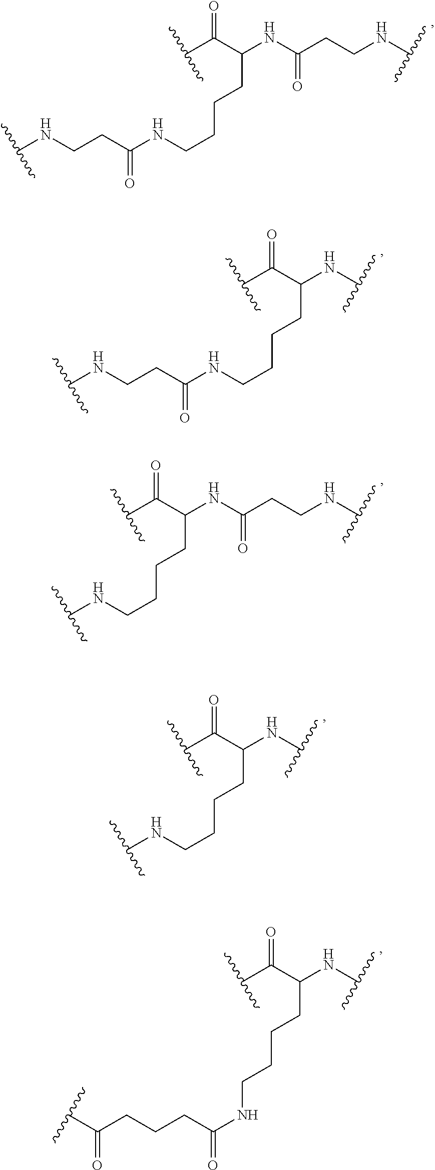 [SMILES: *NCCC(=O)NCCCCC(NC(=O)CCN*)C(*)=O ; *NCCC(=O)NCCCCC(N*)C(*)=O ; *NCCCCC(NC(=O)CCN*)C(*)=O ; *NCCCCC(N*)C(*)=O ; *NC(CCCCNC(=O)CCCC(*)=O)C(*)=O]

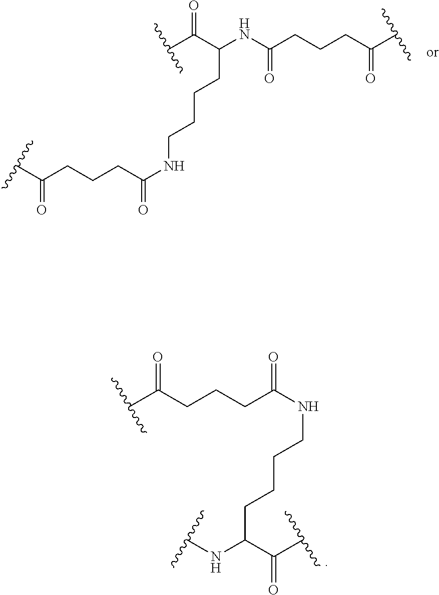 [SMILES: *C(=O)CCCC(=O)NCCCCC(NC(=O)CCCC(*)=O)C(*)=O ; *NC(CCCCNC(=O)CCCC(*)=O)C(*)=O]

### Tetravalent Linkers (A²)

In some embodiments, a tetravalent linker A² is a moiety containing 1 to 100 atoms selected from H, C, N, O, and S, configured to bond to four linkers (e.g., two L² and two L³). In some embodiments, A² is a diamino acid or a dipeptide. In some embodiments, A² is an amino acid or a peptide. In some embodiments, A² is a polypeptide. In some embodiments, A² is an amino acid or a diamino acid that is substituted with one or more arms (e.g., alkyl or heteroalkyl chains, optionally substituted with, or interrupted by, one or more groups independently selected from carbonyls (—C(O)—), ethers (—O—), amines (e.g., —NH— or —N(CH₃)—), or amides (e.g., —C(O)NH—, —C(O)N(CH₃)—, —NHC(O)—, or —N(CH₃)C(O)—, alternatively referred to as amide linkers). In some embodiments, A² is tetravalent heteroalkyl radical. In some embodiments, A² is a tetravalent heteroaryl, heteroaralkyl, aryl, aralkyl, heteroalkyl-aryl, heteroalkyl-heteroaryl radical.

In some embodiments, A² is a tetravalent linker comprising arms B¹, B², B³, and/or B⁴, wherein the tetravalent linker is configured to form an amide bond with four adjacent groups (e.g., L² or L³). In some embodiments, a tetravalent amide linker can be a chemical group having four valencies (or capable of forming bonds to four linkers), and generally composed of alkyl, carbonyl, and amine groups, and preferably capable of forming an amide bond with an adjacent group (e.g., L², L³). In some embodiments, the tetravalent amide linker comprises one or two central amino acid residues (e.g., a lysine, a glutamine, glutamate, or a combination thereof), wherein the amino acid forms a bond with an adjacent group (e.g., L², L³), optionally bridged via a linking arm and/or a spanner. Preferably, a tetravalent radical as described herein conjoins two payloads (e.g., two protease-cleavable payloads (P)) with two target protein binders; or two payloads with a target protein binder and a MOD; or a payload and a MOD with two target protein binders.

In some embodiments, A² has the following structure:

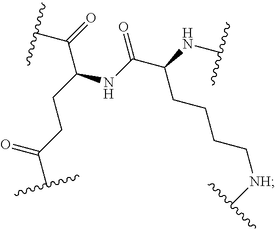 [SMILES: *NCCCC[C@H](N*)C(=O)N[C@@H](CCC(*)=O)C(*)=O]

### Physicochemical or Pharmacokinetic Modulators (MOD)

In some embodiments, the present disclosure provides conjugates comprising a physicochemical or pharmacokinetic modulator (“MOD”). In some embodiments, MOD is a physicochemical modulator. In some embodiments, MOD is a pharmacokinetic modulator. Generally, these terms are used interchangeably unless otherwise specified. A group MOD may be connected to the rest of the conjugate via a linker (e.g., a stable linker). In some embodiments, MOD is connected to EL via a linker (e.g., L⁴). In some embodiments, MOD is bonded to A¹ via a linker (e.g., L², L³). In some embodiments, MOD is a charged or a polar group. Examples of charged or polar groups include hydroxides and alcohols, carboxylates and carboxylic acids, aminiums and amines, guanidiniums and guanidines, and the like. In some embodiments, MOD is or comprises an amine. In some embodiments, MOD is a polyamine group. In some embodiments, MOD is —NR₂ or —NR₃⁺. In some embodiments, MOD is —C₁₋₆ alkyl-NR₂ or —C₁₋₆ alkyl-NR₃⁺ (e.g., —C₁₋₆ alkyl-NH₂, —C₁₋₆ alkyl-NHCH₃, —C₁₋₆ alkyl-N(CH₃)₂, or —C₁₋₆ alkyl-N(CH₃)₃⁺). In some embodiments, MOD is —C₁₋₆ alkyl-NHC(═NH⁺)NH₂. In some embodiments, MOD is a polyacid group. In some embodiments, MOD is —COOH or —COO—. In some embodiments, MOD is —C₁₋₆ alkyl-COOR (e.g., —C₁₋₆ alkyl-COOH, —C₁₋₆ alkyl-COO⁻, —C₁₋₆ alkyl-COO⁻Na⁺, or —C₁₋₆ alkyl-COOCH₃). In some embodiments, MOD is a group that is converted to a polar or charged group in vivo (e.g., MOD is an alkyl ester, which is cleaved in vivo to an alkyl acid, thereby reducing membrane permeability). In some embodiments, MOD is —OH or —O⁻. In some embodiments, MOD is an amino acid. In some embodiments, MOD is a basic amino acid. In some embodiments, MOD is Arg, His, or Lys. In some embodiments, MOD is an acidic amino acid. In some embodiments, MOD is Glu or Asp. In some embodiments, MOD is an unnatural amino acid (e.g., D-Glu or D-Asp). In some embodiments, MOD is a polar amino acid (e.g., Ser, Thr, Asn, Gln). In some embodiments, MOD is a PEG or polysarcosine group.

### Pharmaceutically Acceptable Salts

In one aspect, compounds described herein are in the form of pharmaceutically acceptable salts. As well, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

In some embodiments, pharmaceutically acceptable salts are obtained by reacting a conjugate described herein with an acid. In some embodiments, the conjugate described herein (e.g., free base form) is basic and is reacted with an organic acid or an inorganic acid. Inorganic acids include, but not limited to, hydrochloric acid, hydrobromic acid, sulfuric acid, phosphoric acid, nitric acid, and metaphosphoric acid. Organic acids include, but not limited to, 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (-L); malonic acid; mandelic acid (DL); methanesulfonic acid; naphthalene-1,5-disulfonic acid; naphthalene-2-sulfonic acid; nicotinic acid; oleic acid; oxalic acid; palmitic acid; pamoic acid; phosphoric acid; proprionic acid; pyroglutamic acid (− L); salicylic acid; sebacic acid; stearic acid; succinic acid; sulfuric acid; tartaric acid (+ L); thiocyanic acid; toluenesulfonic acid (p); and undecylenic acid.

In some embodiments, pharmaceutically acceptable salts of the inventive compounds especially include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, benzenesulphonic acid, toluenesulphonic acid, naphthalenedisulphonic acid, formic acid, acetic acid, trifluoroacetic acid, propionic acid, succinic acid, fumaric acid, maleic acid, lactic acid, tartaric acid, malic acid, citric acid, gluconic acid, benzoic acid, and embonic acid.

In some embodiments, a conjugate described herein is prepared as a chloride salt, sulfate salt, bromide salt, mesylate salt, maleate salt, citrate salt or phosphate salt.

In some embodiments, pharmaceutically acceptable salts are obtained by reacting a conjugate described herein with a base. In some embodiments, the conjugate described herein is acidic and is reacted with a base. In such situations, an acidic proton of the conjugate described herein is replaced by a metal ion, e.g., lithium, sodium, potassium, magnesium, calcium, or an aluminum ion. In some cases, compounds described herein coordinate with an organic base, such as, but not limited to, ethanolamine, diethanolamine, triethanolamine, tromethamine, meglumine, N-methylglucamine, dicyclohexylamine, tris(hydroxymethyl)methylamine. In other cases, compounds described herein form salts with amino acids such as, but not limited to, arginine, lysine, and the like. Acceptable inorganic bases used to form salts with compounds that include an acidic proton, include, but not limited to, aluminum hydroxide, calcium hydroxide, potassium hydroxide, sodium carbonate, potassium carbonate, sodium hydroxide, lithium hydroxide, and the like. In some embodiments, the compounds provided herein are prepared as a sodium salt, calcium salt, potassium salt, magnesium salt, meglumine salt, N-methylglucamine salt or ammonium salt. In some embodiments, the compounds provided herein are prepared as a sodium salt, e.g., a monosodium salt, a disodium salt, a trisodium salt, or a tetrasodium salt. In some embodiments, the salt is a disodium or trisodium salt. In some embodiments, the salt is a trifluoroacetate (TFA) salt, e.g., a mono-TFA, di-TFA, tri-TFA, or tetra-TFA salt.

In some embodiments, pharmaceutically acceptable salts of the inventive compounds also include salts derived from conventional bases, by way of example alkali metal salts (e.g. sodium and potassium salts), alkaline earth metal salts (e.g. calcium and magnesium salts), zinc salts and ammonium salts derived from ammonia or organic amines having 1 to 20 carbon atoms, by way of example ethylamine, diethylamine, triethylamine, N,N-ethyldiisopropylamine, monoethanolamine, diethanolamine, triethanolamine, dimethylaminoethanol, diethylaminoethanol, tris(hydroxymethyl)aminomethane, choline, benzalkonium, procaine, dibenzylamine, dicyclohexylamine, N-methylmorpholine, N-methylpiperidine, arginine, lysine, and 1,2-ethylenediamine.

It should be understood that a reference to a pharmaceutically acceptable salt includes the solvent addition forms. In some embodiments, solvates contain either stoichiometric or non-stoichiometric amounts of a solvent, and are formed during the process of crystallization with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

In some embodiments, invention is described as the forms of the inventive compounds which form a complex in the solid or liquid state by coordination with solvent molecules. Hydrates are a specific form of the solvates in which the coordination is with water. Solvates preferred in the context of the present invention are hydrates.

The methods and formulations described herein include the use of N-oxides (if appropriate), or pharmaceutically acceptable salts of compounds having the structure of any one of the formulae disclosed herein, as well as active metabolites of these compounds having the same type of activity.

In some embodiments, sites on the organic radicals (e.g. alkyl groups, aromatic rings) of compounds of formulae disclosed herein are susceptible to various metabolic reactions. Incorporation of appropriate substituents on the organic radicals will reduce, minimize or eliminate this metabolic pathway. In specific embodiments, the appropriate substituent to decrease or eliminate the susceptibility of the aromatic ring to metabolic reactions is, by way of example only, a halogen, deuterium, an alkyl group, a haloalkyl group, or a deuteroalkyl group.

In another embodiment, the compounds described herein are labeled isotopically (e.g. with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine chlorine, iodine, phosphorus, such as, for example, ², ³H, ¹³C, ¹⁴C, ¹⁵N, ¹⁸O, ¹⁷O, ³⁵S, ¹⁸F, ³⁶Cl, ¹²³I, ¹²⁴I, ¹²⁵I, ¹³¹I, ³²P and ³³P. In one aspect, isotopically-labeled compounds described herein, for example those into which radioactive isotopes such as ³H and ¹⁴C are incorporated, are useful in drug or substrate tissue distribution assays. In one aspect, substitution with isotopes such as deuterium affords certain therapeutic advantages resulting from greater metabolic stability, such as, for example, increased in vivo half-life or reduced dosage requirements.

In some embodiments, the present invention also encompasses all suitable isotopic variants of the inventive compounds. An isotopic variant of an inventive compound is understood here to mean a compound in which at least one atom within the inventive compound has been exchanged for another atom of the same atomic number, but with a different atomic mass than the atomic mass which usually or predominantly occurs in nature. Examples of isotopes which can be incorporated into an inventive compound are those of hydrogen, carbon, nitrogen, oxygen, phosphorus, sulfur, fluorine, chlorine, bromine and iodine, such as ²H (deuterium), ³H (tritium), ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I. Particular isotopic variants of an inventive compound, especially those in which one or more radioactive isotopes have been incorporated, may be beneficial, for example, for the examination of the mechanism of action or of the active ingredient distribution in the body; due to comparatively easy preparability and detectability, particularly compounds labelled with ³H, ¹⁴C, or ¹⁸F isotopes are suitable for the purpose. In addition, the incorporation of isotopes, for example of deuterium, can lead to particular therapeutic benefits as a consequence of greater metabolic stability of the compound, for example an extension of the half-life in the body or a reduction in the active dose required; such modifications of the inventive compounds may therefore possibly also constitute a preferred embodiment of the present invention. Isotopic variants of the inventive compounds can be prepared by commonly used processes known to those skilled in the art, for example by the methods described further down and the procedures described in the working examples, by using corresponding isotopic modifications of the respective reagents or starting compounds.

In some embodiments, the compounds disclosed herein possess one or more stereocenters and each stereocenter exists independently in either the R or S configuration. In some embodiments, the conjugate described herein exists in the R configuration. In some embodiments, the conjugate described herein exists in the S configuration. The compounds presented herein include all diastereomeric, individual enantiomers, atropisomers, and epimeric forms as well as the appropriate mixtures thereof. The compounds and methods provided herein include all cis, trans, syn, anti, entgegen (E), and zusammen (Z) isomers as well as the appropriate mixtures thereof.

Individual stereoisomers are obtained, if desired, by methods such as, stereoselective synthesis or the separation of stereoisomers by chiral chromatographic columns or the separation of diastereomers by either non-chiral or chiral chromatographic columns or crystallization and recrystallization in a proper solvent or a mixture of solvents. In certain embodiments, compounds disclosed herein are prepared as their individual stereoisomers by reacting a racemic mixture of the compound with an optically active resolving agent to form a pair of diastereoisomeric compounds/salts, separating the diastereomers and recovering the optically pure individual enantiomers. In some embodiments, resolution of individual enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In some embodiments, stereoisomers are obtained by stereoselective synthesis.

### Prodrugs

In some embodiments, compounds described herein are prepared as prodrugs. A “prodrug” refers to an agent that is converted into the parent drug in vivo. Prodrugs are often useful because, in some situations, they are easier to administer than the parent drug. They are, for instance, bioavailable by oral administration whereas the parent is not. Further or alternatively, the prodrug also has improved solubility in pharmaceutical compositions over the parent drug. In some embodiments, the design of a prodrug increases the effective water solubility. An example, without limitation, of a prodrug is a compound described herein, which is administered as an ester (the “prodrug”) but then is metabolically hydrolyzed to provide the active entity. A further example of a prodrug is a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

In some embodiments, a prodrug is activated upon enzymatic cleavage (e.g., a tumor-associated enzyme such as neutrophil elastase). In some embodiments, the prodrug contains a peptide sequence that is recognized by a given enzyme. In some embodiments, a prodrug is selectively cleaved by a specific enzyme.

In some embodiments, a prodrug further comprises a non-peptidic prodrug moiety (e.g., an ester) which is released in vivo. Non-peptidic prodrug moiety indicates that the bond being metabolized or broken to release the active agent is not a peptide bond (—C(O)—NH—). In some embodiments, the non-peptidic prodrug moiety is an alkyl ester (including substitutions such as alkyl substitutions on the alkyl group). In some embodiments, the non-peptidic prodrug moiety is an ester of an amino acid, such as an aspartate or glutamate residue. In some embodiments, the alkyl ester prodrug is indicated by Asp* or Glu*, indicating an alkyl (e.g., substituted alkyl) group is masking the carboxylic acid moiety of the side-chain. In some embodiments, provided herein are prodrug compounds wherein an alkylamine (—C₁₋₆alkyl-NR₂) or alkylaminium (—C₁₋₆alkyl-NR₃⁺) group is released in vivo (where “R” as used here is hydrogen or C₁₋₆alkyl) from an amino acid ester (e.g., Asp* or Glu*) to liberate the acid moiety. In some embodiments, a non-peptidic prodrug moiety (e.g., an ester (e.g., an alkylamine or alkylaminium ester)) is released (i.e., cleaved) in plasma, while an enzymatically cleavable moiety remains intact (e.g., when the cleaving enzyme is not present or abundant). In some embodiments, the prodrug ester is slowly cleaved in plasma with no detectable release of the enzymatically cleavable moiety or the payload (e.g., the cytotoxic or cytostatic ligand) attached thereto. In some embodiments, the non-peptidic (i.e., ester) prodrug moiety is cleaved before the peptidic prodrug moiety. In some embodiments, the non-peptidic (i.e., ester) prodrug moiety is cleaved independently of protease activity (e.g., non-proteolytic cleavage). In some embodiments, the non-peptidic (i.e., ester) prodrug moiety is cleaved to a degree of about 50% (f 5%) slowly. As used here, “slowly” means the prodrug is cleaved to a degree of about 50% after at least two hours in plasma. In some embodiments, slow cleavage constitutes about 50% prodrug release (i.e., release of the ester moiety to yield the active carboxylic acid) after about 2, about 3, about 4, about 5, about 6, about 8, about 10, or about 12 hours in plasma. In some embodiments, slow cleavage constitutes about 50% prodrug release after about 2 hours to about 12 hours in plasma. In some embodiments, an ester prodrug is cleaved to a degree of about 50% after about 2 hours to about 6 hours in plasma. In some embodiments, an ester prodrug is cleaved to a degree of about 50% after about 2 hours to about 4 hours in plasma. As described previously, the non-peptidic (i.e., ester) prodrug moiety is cleaved independently (e.g., in the absence or presence) of proteolytic enzymes such as cathepsin B, legumain, or neutrophil elastase. As shown in FIG. 2 and FIG. 3, in some embodiments, the ester prodrug moiety is cleaved to a degree of about 50% after about 3 or 4 hours (and within about 6 hours) of administration in vivo. The degradation product of such a prodrug is, in some embodiments, the parent conjugate sans alkyl ester, meaning the enzymatically cleavable portion, cytotoxic or cytostatic moiety, linker/spacer, and integrin binder remain intact. In some embodiments, enzymatic cleavage takes place after release of the ester prodrug moiety. In some embodiments, the protease responsible for cleaving the enzymatically cleavable moiety recognizes the free Asp or Glu residue, but not the Asp* or Glu* prodrug ester.

Prodrugs of the compounds described herein include, but not limited to, esters, ethers, carbonates, thiocarbonates, N-acyl derivatives, N-acyloxyalkyl derivatives, N-alkyloxyacyl derivatives, quaternary derivatives of tertiary amines, N-Mannich bases, Schiff bases, amino acid conjugates, phosphate esters, and sulfonate esters. See for example Design of Prodrugs, Bundgaard, A. Ed., Elsevier, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference. In some embodiments, a hydroxyl group in the compounds disclosed herein is used to form a prodrug, wherein the hydroxyl group is incorporated into an acyloxyalkyl ester, alkoxycarbonyloxyalkyl ester, alkyl ester, aryl ester, phosphate ester, sugar ester, ether, and the like. In some embodiments, a hydroxyl group in the compounds disclosed herein is a prodrug wherein the hydroxyl is then metabolized in vivo to provide a carboxylic acid group. In some embodiments, a carboxyl group is used to provide an ester or amide (e.g., the prodrug), which is then metabolized in vivo to provide a carboxylic acid group. In some embodiments, compounds described herein are prepared as alkyl ester prodrugs.

Prodrug forms of the herein described compounds, wherein the prodrug is metabolized in vivo to produce a conjugate described herein as set forth herein are included within the scope of the claims. In some cases, some of the herein-described compounds is a prodrug for another derivative or active compound.

In some embodiments, any one of the hydroxyl group(s), amino group(s), or carboxylic acid group(s) are functionalized in a suitable manner to provide a prodrug moiety. In some embodiments, the prodrug moiety is as described above.

### Pharmaceutical Compositions

The present invention provides a pharmaceutical composition comprising a compound of formulae disclosed herein, or a pharmaceutically acceptable salt thereof; or a stereoisomer or mixture of stereoisomers thereof; and a pharmaceutically acceptable excipient.

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the compounds described herein are administered either alone or in combination with pharmaceutically acceptable carriers, excipients or diluents, in a pharmaceutical composition. Administration of the compounds and compositions described herein can be effected by any method that enables delivery of the compounds to the site of action. These methods include, though not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route may depend upon for example the condition and disorder of the recipient. By way of example only, compounds described herein can be administered locally to the area in need of treatment, by for example, local infusion during surgery, topical application such as creams or ointments, injection, catheter, or implant. The administration can also be by direct injection at the site of a diseased tissue or organ.

In some embodiments, pharmaceutical compositions suitable for oral administration are presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. In some embodiments, the active ingredient is presented as a bolus, electuary or paste.

Pharmaceutical compositions which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. In some embodiments, the tablets are coated or scored and are formulated to provide slow or controlled release of the active ingredient therein. All formulations for oral administration should be in dosages suitable for such administration. The push-fit capsules can contain the active ingredients in admixture with filler such as lactose, binders such as starches, or lubricants such as talc or magnesium stearate and, optionally, stabilizers. In soft capsules, the active compounds may be dissolved or suspended in suitable liquids, such as fatty oils, liquid paraffin, or liquid polyethylene glycols. In some embodiments, stabilizers are added. Dragee cores are provided with suitable coatings. For this purpose, concentrated sugar solutions may be used, which may optionally contain gum arabic, talc, polyvinyl pyrrolidone, carbopol gel, polyethylene glycol, or titanium dioxide, lacquer solutions, and suitable organic solvents or solvent mixtures. Dyestuffs or pigments may be added to the tablets or Dragee coatings for identification or to characterize different combinations of active compound doses.

In some embodiments, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions, or emulsions in oily or aqueous vehicles, and may contain formulatory agents such as suspending, stabilizing, or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Pharmaceutical compositions for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical compositions may also be formulated as a depot preparation. Such long-acting formulations may be administered by implantation (for example subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

Pharmaceutical compositions may be administered topically, that is by non-systemic administration. This includes the application of a compound of the present invention externally to the epidermis or the buccal cavity and the instillation of such a compound into the ear, eye and nose, such that the compound does not significantly enter the blood stream. In contrast, systemic administration refers to oral, intravenous, intraperitoneal, and intramuscular administration.

Pharmaceutical compositions suitable for topical administration include liquid or semi-liquid preparations suitable for penetration through the skin to the site of inflammation such as gels, liniments, lotions, creams, ointments, or pastes, and drops suitable for administration to the eye, ear or nose. The active ingredient may comprise, for topical administration, from 0.001% to 10% w/w, for instance from 1% to 2% by weight of the formulation.

Pharmaceutical compositions for administration by inhalation are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, pharmaceutical preparations may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

In addition to the ingredients particularly mentioned above, the compounds and compositions described herein may include other agents conventional in the art having regard to the type of formulation in question, for example those suitable for oral administration may include flavoring agents.

### Compounds for Use in Treating Diseases or Disorders

In some embodiments, the present invention provides compounds of formulae disclosed herein, or a pharmaceutically acceptable salt thereof, or a stereoisomer or mixture of stereoisomers thereof, for the treatment of a disease or disorder.

In some embodiments, the present invention provides compounds of formulae disclosed herein, or a pharmaceutically acceptable salt thereof, or a stereoisomer or mixture of stereoisomers thereof, for use in the manufacture of a medicament for treating a disease or disorder described herein.

In some embodiments, the present invention provides compounds of formulae disclosed herein, or a pharmaceutically acceptable salt thereof, or a stereoisomer or mixture of stereoisomers thereof, for the treatment of a hyperproliferative disorder.

In some embodiments, the present invention provides compounds of formulae disclosed herein, or a pharmaceutically acceptable salt thereof, or a stereoisomer or mixture of stereoisomers thereof, for the treatment of a cancer.

In some embodiments, the present invention provides compounds of formulae disclosed herein, or a pharmaceutically acceptable salt thereof, or a stereoisomer or mixture of stereoisomers thereof, wherein for the treatment of an autoimmune disorder.

### Methods of Dosing and Treatment Regimens

In some embodiments, the present invention provides a method of treating a disease or disorder in a subject, comprising administering a therapeutically effective amount of a compound of formulae disclosed herein, or a pharmaceutically acceptable salt thereof, or a stereoisomer or mixture of stereoisomers thereof, or a pharmaceutical composition thereof, to an individual in need thereof. In some embodiments, the compound used in the method of treating a disease or disorder is a compound of Formula (I), Formula (II), Formula (III), Formula (IV), Formula (V), Formula (VI), Formula (VII), Formula (VIII), or Formula (IX), or a pharmaceutically acceptable salt thereof, or a stereoisomer or mixture of stereoisomers thereof.

In some embodiments, the present invention provides a method of treating a hyperproliferative disorder in a subject, comprising administering a therapeutically effective amount of a compound of formulae disclosed herein, or a pharmaceutically acceptable salt thereof, or a stereoisomer or mixture of stereoisomers thereof, or a pharmaceutical composition thereof, to an individual in need thereof.

In some embodiments, the present invention provides a method of treating a cancer in a subject, comprising administering a therapeutically effective amount of a compound of formulae disclosed herein, or a pharmaceutically acceptable salt thereof, or a stereoisomer or mixture of stereoisomers thereof, or a pharmaceutical composition thereof, to an individual in need thereof.

In some embodiments, the present invention provides a method of treating an autoimmune disorder in a subject, comprising administering a therapeutically effective amount of a compound of formulae disclosed herein, or a pharmaceutically acceptable salt thereof, or a stereoisomer or mixture of stereoisomers thereof, or a pharmaceutical composition thereof, to an individual in need thereof.

### EXAMPLES

As used above, and throughout the description of the invention, the following abbreviations, unless otherwise indicated, shall be understood to have the following meanings:

### Abbreviations

Abu: γ-amino butyric acid
ACN: acetonitrile
Boc: tert.-butyloxycarbonyl
Bzl: benzyl
DCM: dichloromethane
DIEA: N, N diisopropyl ethyl amine (Hünig's base)
DMAP: dimethylamino pyridine
DMF: dimethyl formamide
DMSO: dimethyl sulfoxide
EDCI: 1-Ethyl-3-(3-dimethylaminopropyl)carbodiimide
ee: enantiomeric excess
Fmoc: fluorenyl-9-methoxycarbonyl
HATU: 2-(1H-7-Azabenzotriazol-1-yl)-1,1,3,3-tetramethyluronium hexafluorophosphate
HBTU: Hexafluorophosphate Benzotriazole Tetramethyl Uronium
HPLC: high-performance liquid chromatography
MTBE: methyl tert.-butyl ether
NMP: N-methyl pyrrolidone
RP: reverse phase
rt: room temperature
T3P: propanephosphonic acid anhydride
TFA: trifluoroacetic acid
THF: tetrahydrofuran
TLC: thin-layer chromatography

The following examples are provided for illustrative purposes only and not to limit the scope of the claims provided herein.

### Analytical Methods (LC-MS)

Method 1 (LC-MS): Instrument: Waters ACQUITY SQD UPLC System; Column: Waters Acquity UPLC HSS T3 1.8μ 50×1 mm; Eluent A: 11 Water+0.25 mL 99% formic acid, Eluent B: 11 acetonitrile+0.25 mL 99% formic acid; Gradient: 0.0 min 90% A→1.2 min 5% A→2.0 min 5% A Stove: 50° C.; Flow: 0.40 mL/min; UV-Detection: 208-400 nm.

Method 2 (LC-MS): System MS: Thermo Scientific FT-MS; System UHPLC+: Thermo Scientific UltiMate 3000; Column: Waters, HSST3, 2.1×75 mm, C18 1.8 μm; Eluent A: 11 Water+0.01% Formic acid; Eluent B: 11 Acetonitrile+0.01% Formic acid; Gradient: 0.0 min 10% B→2.5 min 95% B→3.5 min 95% B; Oven: 50° C.; Flow: 0.90 ml/min; UV-Detection: 210 nm/Optimum Integration Path 210-300 nm.

Method 3 (LC-MS): System MS: Waters TOF instrument; System UPLC: Waters Acquity I-CLASS; Column: Waters, HSST3, 2.1×50 mm, C18 1.8 μm; Eluent A: 11 Water+0.01% Formic acid; Eluent B: 11 Acetonitrile+0.01% Formic acid; Gradient: 0.0 min 2% B→0.5 min 2% B→7.5 min 95% B→10.0 min 95% B; Oven: 50° C.; Flow: 1.00 ml/min; UV-Detection: 210 nm.

Method 4 (LC-MS): System MS: Thermo Scientific FT-MS; System UHPLC+: Thermo Scientific Vanquish; Column: Waters, HSST3, 2.1×75 mm, C18 1.8 μm; Eluent A: 11 Water+0.01% Formic acid; Eluent B: 11 Acetonitrile+0.01% Formic acid; Gradient: 0.0 min 10% B→2.5 min 95% B→3.5 min 95% B; Oven: 50° C.; Flow: 0.90 ml/min; UV-Detection: 210 nm.

Method 5 (LC-MS): System MS: Waters TOF instrument; System UPLC: Waters Acquity I-CLASS; Column: Waters Acquity UPLC HSS T3 1.8 μm 50×1 mm; Eluent A: 11 Water+0.100 ml 99% Formic acid, Eluent B: 11 Acetonitrile+0.100 ml 99% Formic acid; Gradient: 0.0 min 90% A→0.1.2 min 5% A→2.0 min 5% A Oven: 50° C.; Flow: 0.40 ml/min; UV-Detection: 210 nm.

Method 6 (LC-MS): System MS: Waters TOF instrument; System UPLC: Waters Acquity I-CLASS; Column: Waters Acquity UPLC HSS T3 1.8 μm 50×1 mm; Eluent A: 11 Water+0.100 ml 99% Formic acid, Eluent B: 11 Acetonitrile+0.100 ml 99% Formic acid; Gradient: 0.0 min 95% A→6.0 min 5% A→7.5 min 5% A Oven: 50° C.; Flow: 0.35 ml/min; UV-Detection: 210 nm.

Method 7 (LC-MS): Instrument: Waters Single Quad MS System; Instrument Waters UPLC Acquity; Column: Waters BEH C18 1.7μ 50×2.1 mm; Eluent A: 11 Water+1.0 mL (25% ig Ammonia)/L, Eluent B: 11 Acetonitrile; Gradient: 0.0 min 92% A→0.1 min 92% A→1.8 min 5% A→3.5 min 5% A; Oven: 50° C.; Flow: 0.45 mL/min; UV-Detection: 210 nm.

Method 8 (LC-MS): Instrument: Waters ACQUITY SQD UPLC System; Column: Waters Acquity UPLC HSS T3 1.8 μm 50×1 mm; Eluent A: 11 Water+0.25 ml 99% Formic acid, Eluent B: 11 Acetonitrile+0.25 ml 99% Formic acid; Gradient: 0.0 min 90% A→1.2 min 5% A→2.0 min 5% A Oven: 50° C.; Flow: 0.40 ml/min; UV-Detection: 210 nm.

Method 9 (LC-MS): System MS: Waters TOF instrument; System UPLC: Waters Acquity I-CLASS; Column: Waters Acquity UPLC HSS T3, 2.1×150 mm, 1.8 μm; Eluent A: 1 L Water+0.100 mL 99% trifluoroacetic acid, Eluent B: 1 L Acetonitrile+0.100 mL 99% trifluoroacetic acid; Gradient: 0.0 min 5% B→1 min 5% B→13 min 95% B→15 min 95% B; Oven: 50° C.; Flow: 0.60 mL/min; UV-Detection: 210 nm.

### Synthesis Examples

### Building Blocks—Intermediates

### Example S1: Preparation of (3R)-3-{[(4-aminophenyl)carbamoyl]amino}-3-{-[({3-[(propyl carbamoyl)amino]phenyl}sulfonyl)amino]phenyl}propanoic acid (Intermediate 1)

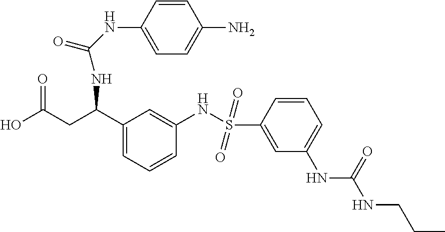 [SMILES: CCCNC(=O)Nc1cccc(S(=O)(=O)Nc2cccc([C@@H](CC(=O)O)NC(=O)Nc3ccc(N)cc3)c2)c1]

The synthesis of Intermediate 1 has been described in WO2020/094471. ¹H-NMR (500 MHz, D4-methanol): δ=0.93 (t, 3H), 1.5 (m, 2H), 2.74 (d, 2H), 3.1 (dt, 2H), 5.15 (t, 1H), 6.68 (d, 2H), 6.85 (d, 1H), 7.05 (d, 1H), 7.1 (d, 1H), 7.13 (t, 1H), 7.28-7.4 (m, 3H), 7.6 (s, 1H), 7.66 (d, 1H).

### Example S2: Preparation of (3R)-3-{[(4-{[(4-nitrophenoxy)carbonyl]amino}phenyl)carbamoyl]amino}-3-{3-[({3-[(propylcarbamoyl)amino]phenyl}sulfonyl)amino]phenyl}propanoic acid (Intermediate 2)

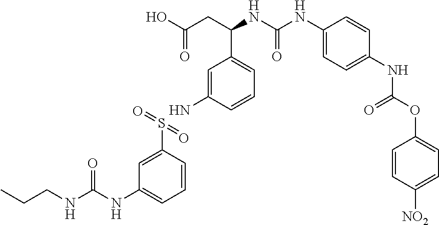

The synthesis of Intermediate 2 has been described in WO2020/094471.

### Example S3: Preparation of (2S)-1-[(19S)-19-(2-tert-butoxy-2-oxoethyl)-2,2-dimethyl-4,17,20-trioxo-3,8,11,14-tetraoxa-5,18-diazaicosan-20-yl]pyrrolidine-2-carboxylic acid (Intermediate 3)

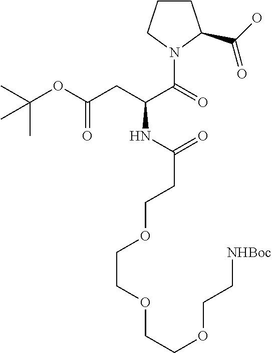 [SMILES: CC(C)(C)OC(=O)C[C@H](NC(=O)CCOCCOCCOCCNC(=O)OC(C)(C)C)C(=O)N1CCC[C@H]1C(=O)O]

Intermediate 3 was synthesized using classical methods of peptide synthesis starting with the coupling of Z-Asp(OtBu)-OH with benzyl L-prolinate hydrochloride (1:1) in THF in the presence of T3P and DIPEA and subsequent removal of the Z-protecting group as well as the benzyl ester by hydrogenolysis over Pd/C to give (2S)-1-[(2S)-2-amino-4-tert-butoxy-4-oxobutanoyl]pyrrolidine-2-carboxylic acid. This partially protected dipeptide was acylated with tert-butyl{2-[2-(2-{3-[(2,5-dioxopyrrolidin-1-yl)oxy]-3-oxopropoxy}ethoxy)ethoxy]ethyl}carbamate to give the title compound. Tert-butyl{2-[2-(2-{3-[(2,5-dioxopyrrolidin-1-yl)oxy]-3-oxopropoxy}ethoxy) ethoxy]ethyl}carbamate was previously prepared by reacting 2,2-dimethyl-4-oxo-3,8,11,14-tetraoxa-5-azaheptadecan-17-oic acid with N-Hydroxysuccinimide in dioxane in the presence of EDCI. LC-MS: Rₜ=0.81 min; MS (ESIpos): m/z=590 [M+H]⁺.

### Example S4: Preparation of (4S)-4,11-diethyl-3,14-dioxo-3,4,12,14-tetrahydro-1H-pyrano[3′,4′:6,7]indolizino[1,2-b]quinolin-4-yl L-valinate⋅trifluoroacetic acid (1:1) (Intermediate 4)

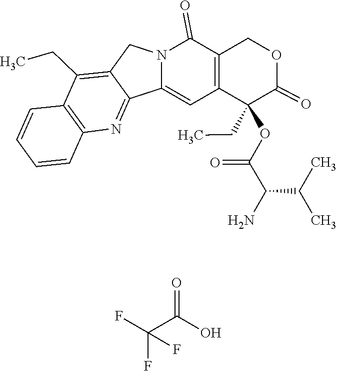 [SMILES: CCc1c2c(nc3ccccc13)-c1cc3c(c(=O)n1C2)COC(=O)[C@@]3(CC)OC(=O)[C@@H](N)C(C)C ; O=C(O)C(F)(F)F]

2.59 g (10.6 mmol) of N-(tert-butoxycarbonyl)-valine-N-carboxyanhydride and 0.5 g of 4-(N,N-dimethylamino)-pyridine were added to a stirred suspension of 2 g (5.3 mmol) of (4S)-4,11-diethyl-4-hydroxy-1H-pyrano[3′,4′:6,7]indolizino[1,2-b]quinoline-3,14(4H,12H)-dione in 150 ml of absolute dichloromethane. The mixture was stirred at rt for 20 h and subsequently concentrated in vacuo. 8 ml ACN were added to the residue and subsequently 5 mL diethyl ether. The mixture was filtrated and the remaining residue was dried in vacuo. 2964 mg (92% yield) of the protected intermediate were obtained. LC-MS: Rt=1.19 min; MS (ESIpos): m/z=576 (M+H)⁺. Next, 2964 mg (5.15 mmol) of this Boc-protected intermediate compound in 6 ml of dichloromethane and 60 ml of anhydrous trifluoroacetic acid was stirred for 30 min. at rt and subsequently sonicated for 1 h. After concentrating in vacuo the product was lyophilized from a mixture of acetonitrile/water. 3.622 g (quant) of Intermediate 4 obtained. LC-MS: Rt=0.68 min; MS (ESIpos): m/z=476 [M+H]⁺.

### Example S5: Preparation of tert-butyl (19S)-19-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-2,2-dimethyl-4,17-dioxo-3,8,11,14-tetraoxa-5,18-diazahenicosan-21-oate (Intermediate 5)

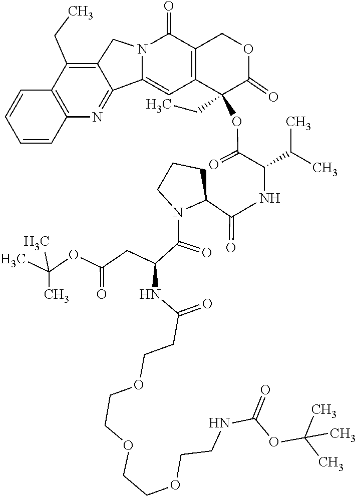 [SMILES: CCc1c2c(nc3ccccc13)-c1cc3c(c(=O)n1C2)COC(=O)[C@@]3(CC)OC(=O)[C@@H](NC(=O)[C@@H]1CCCN1C(=O)[C@H](CC(=O)OC(C)(C)C)NC(=O)CCOCCOCCOCCNC(=O)OC(C)(C)C)C(C)C]

(2S)-1-[(19S)-19-(2-Tert-butoxy-2-oxoethyl)-2,2-dimethyl-4,17,20-trioxo-3,8,11,14-tetraoxa-5,18-diazaicosan-20-yl]pyrrolidine-2-carboxylic acid (Intermediate 3) (50.0 g, 84.8 mmol) was dissolved in 900 ml DMF and the solution was cooled down to 0° C. 1.1 eq 1-(3-Dimethylaminopropyl)-3-ethylcarbodiimidhydrochloride (17.0 g, 88.8 mmol) and 1.3 eq Ethyl cyanohydroxyiminoacetate (14.9 g, 105 mmol) and trifluoroacetic acid-(4S)-4,11-diethyl-3,14-dioxo-3,4,12,14-tetrahydro-1H-pyrano[3′,4′:6,7]indolizino[1,2-b]quinolin-4-yl L-valinate (1/1) (Intermediate 4) (47.6 g, 80.8 mmol) were added and subsequently 3.0 eq (42 ml, 240 mmol) N,N-diisopropylethylamine was added dropwise. The mixture was stirred overnight at 0° C. It was diluted with 4 L EtOAc and the organic layer was washed with 10% aqueous citric acid (2×21), with 10% aqueous NaHCO₃-solution (2×2 l) and with saturated aqueous NaCl-solution (2×31). Subsequently it was dried over Mg₂SO₄, filtered and concentrated. The residue was dissolved in 250 ml DCM and purified using flash chromatography (DCM:MeOH 100:1). Relevant fractions were collected and evaporated in vacuo to yield Intermediate 5 as a yellow foam (72.8 g, 98% purity, 84% yield).

### Example S6: Preparation of 4S)-4,11-diethyl-3,14-dioxo-3,4,12,14-tetrahydro-1H pyrano[3′,4′:6,7]indolizino[1,2-b]quinolin-4-yl N-(3-{2-[2-(2-aminoethoxy)ethoxy]ethoxy}propanoyl)-L-alpha-aspartyl-L-prolyl-L-valinate⋅trifluoroacetic acid (1:1) (Intermediate 6)

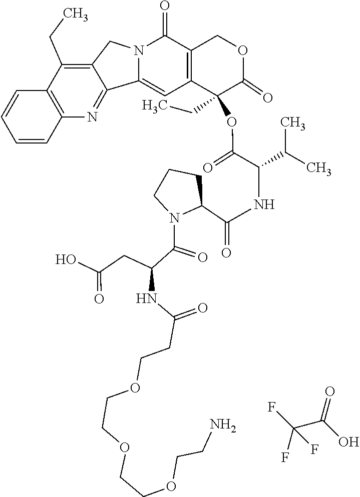 [SMILES: CCc1c2c(nc3ccccc13)-c1cc3c(c(=O)n1C2)COC(=O)[C@@]3(CC)OC(=O)[C@@H](NC(=O)[C@@H]1CCCN1C(=O)[C@H](CC(=O)O)NC(=O)CCOCCOCCOCCN)C(C)C ; O=C(O)C(F)(F)F]

12 g (11.5 mmol) of tert-butyl (19S)-19-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl} pyrrolidine-1-carbonyl]-2,2-dimethyl-4,17-dioxo-3,8,11,14-tetraoxa-5,18-diazahenicosan-21-oate (Intermediate 5) was dissolved in 100 ml of dichloromethane and 40 ml of anhydrous trifluoroacetic acid was added and the solution was stirred for 2 days at rt. After concentrating in vacuo 50 ml toluene were added and again evaporated. The residue was dissolved in 50 ml DCM/MeOH and subsequently poured into 600 ml of diethyl ether. The precipitating product (Intermediate 6) was filtered, washed with diethyl ether and dried in vacuo. (11.7 g, 95% purity, 97% yield).

### Example S7: Preparation of (4S)-4,11-diethyl-3,14-dioxo-3,4,12,14-tetrahydro-1H-pyrano[3′,4′:6,7]indolizino[1,2-b]quinolin-4-yl N-(2,2-dimethyl-4,17-dioxo-3,8,11,14-tetraoxa-5-azaheptadecan-17-yl)-L-alpha-aspartyl-L-prolyl-L-valinate (Intermediate 7)

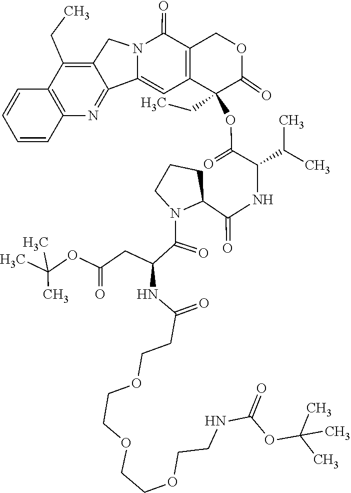 [SMILES: CCc1c2c(nc3ccccc13)-c1cc3c(c(=O)n1C2)COC(=O)[C@@]3(CC)OC(=O)[C@@H](NC(=O)[C@@H]1CCCN1C(=O)[C@H](CC(=O)OC(C)(C)C)NC(=O)CCOCCOCCOCCNC(=O)OC(C)(C)C)C(C)C]

1 g (995 μmol) of (4S)-4,11-diethyl-3,14-dioxo-3,4,12,14-tetrahydro-1H pyrano[3′,4′:6,7]indolizino[1,2-b]quinolin-4-yl N-(3-{2-[2-(2-aminoethoxy)ethoxy]ethoxy}propanoyl)-L-alpha-aspartyl-L-prolyl-L-valinate⋅trifluoroacetic acid (1:1) (Intermediate 6) was dissolved in 11 ml DMF and 1-[(tert-butoxycarbonyl)oxy]pyrrolidine-2,5-dione (214 mg, 995 μmol) and 1 eq N,N-diisopropylethylamine (170 μl, 1000 μmol) were added. The batch was stirred for 20 h at rt. Another 1/2 eq of BOC-OSu and N,N-diisopropylethylamine were added and the mixture was stirred for further 3 h at rt. Then the batch was evaporated to dryness using a rotary evaporator. The residue was separated by HPLC. Relevant fractions were collected and evaporated in vacuo to yield 744 mg (100% purity, 75% yield) of Intermediate 7 as a yellow foam. LC-MS: Rₜ=4.19 min; MS (ESIpos): m/z=991 [M+H]⁺.

### Example S8: Preparation of (4S)-4,11-Diethyl-3,14-dioxo-3,4,12,14-tetrahydro-1H-pyrano[3′,4′:6,7]indolizino[1,2-b]quinolin-4-yl N-[3-(2-{2-[2-(L-lysylamino)ethoxy]ethoxy}ethoxy)propanoyl]-L-alpha-aspartyl-L-prolyl-L-valinate⋅trifluoroacetic acid (1:1) (Intermediate 8)

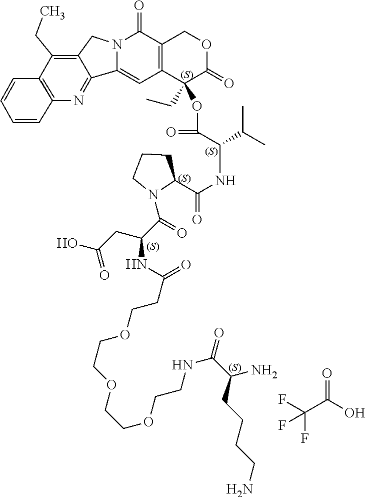

Step 1: (4S)-4,11-diethyl-3,14-dioxo-3,4,12,14-tetrahydro-1H-pyrano[3′,4′:6,7]indolizino[1,2-b]quinolin-4-yl N-(2,2-dimethyl-4,17-dioxo-3,8,11,14-tetraoxa-5-azaheptadecan-17-yl)-L-alpha-aspartyl-L-prolyl-L-valinate (Intermediate 7) (500 mg, 498 μmol) was dissolved in 50 mL DMF and 1.2 eq 2,5-dioxopyrrolidin-1-yl N²,N⁶-bis(tert-butoxycarbonyl)-L-lysinate (265 mg, 597 μmol) as well as 260 μL N,N-diisopropylethylamine were added. After stirring for 2 h at rt the mixture was concentrated in vacuo and the residual was purified by prep. HPLC. Relevant fractions were collected and concentrated in vacuo to yield Boc-Intermediate 8 as a light yellow foam (506 mg, 98% purity, 81% yield). LC-MS: Rₜ=1.93 min; MS (ESIpos): m/z=1218 [M+H]⁺.

Step 2: 506 mg (415 μmol) of Boc-Intermediate 8 was dissolved in 10 ml DCM and 3 ml TFA was added and the reaction mixture was stirred for 30 min at rt. It was concentrated in vacuo, the residue was dissolved in ACN/H₂O and lyophilized to give Intermediate 8 as a light yellow foam. (700 mg (100% purity, quant.). LC-MS: Rₜ=1.77 min; MS (ESIpos): m/z=510 [M+2H]⁻.

### Example S9 (Intermediate 9): Preparation of (4S)-4,11-diethyl-3,14-dioxo-3,4,12,14-tetrahydro-1H-pyrano[3′,4′:6,7]indolizino[1,2-b]quinolin-4-yl N-(3-{2-[2-(2-([N²,N⁶-bis(3-{2-[2-(2-aminoethoxy)ethoxy]ethoxy}propanoyl)-L-lysyl]amino)ethoxy)ethoxy]ethoxy}propanoyl)-L-alpha-aspartyl-L-prolyl-L-valinate⋅trifluoroacetic acid (1/2) (Intermediate 9)

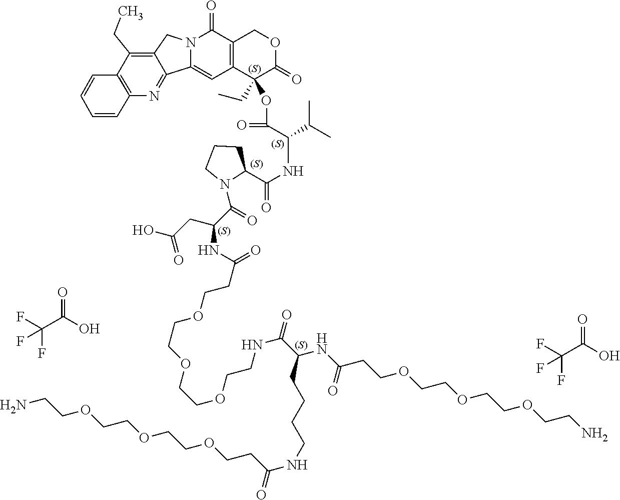

Step 1: (4S)-4,11-Diethyl-3,14-dioxo-3,4,12,14-tetrahydro-1H-pyrano[3′,4′:6,7]indolizino[1,2-b]quinolin-4-yl N-[3-(2-{2-[2-(L-lysylamino)ethoxy]ethoxy}ethoxy)propanoyl]-L-alpha-aspartyl-L-prolyl-L-valinate (Intermediate 8) (70 mg, 62 μmol) was dissolved in 8 mL DMF and 2.2 eq tert-butyl {2-[2-(2-{-[(2,5-dioxopyrrolidin-1-yl)oxy]-3-oxopropoxy}ethoxy)ethoxy]ethyl}carbamate(56.9 mg, 136 μmol) as well as 65 μL N,N-diisopropylethylamine were added. After stirring for 4 h at rt the mixture was concentrated in vacuo and the residual was purified by prep. HPLC. Relevant fractions were collected and concentrated in vacuo to yield (Boc-Intermediate 9) as a light yellow foam (60 mg, 87% purity, 52% yield).

Step 2: 60 mg (37 μmol) of Boc-Intermediate 9 were dissolved in 10 ml DCM and 2 ml TFA was added and the reaction mixture was stirred for 30 min at rt. It was concentrated in vacuo, the residue was dissolved in ACN/H₂O and lyophilized to give Intermediate 9 as a colorless foam. (60 mg, 100% purity, 98% yield). LC-MS: Rₜ=1.86 min; MS (ESIpos): m/z=1425 [M+H]⁺.

### Example S10: Preparation of tert-butyl {2-[2-(2-{3-[(2,5-dioxopyrrolidin-1-yl)oxy]-3-oxopropoxy}ethoxy)ethoxy]ethyl}carbamate (Intermediate 10)

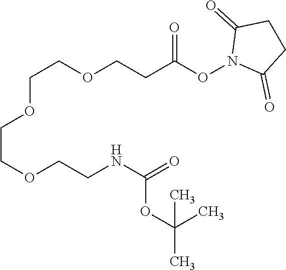 [SMILES: CC(C)(C)OC(=O)NCCOCCOCCOCCC(=O)ON1C(=O)CCC1=O]

To a suspension of 2,2-dimethyl-4-oxo-3,8,11,14-tetraoxa-5-azaheptadecan-17-oic acid (3.00 g, 9.33 mmol) in DCM (50 ml), were added 1-hydroxypyrrolidine-2,5-dione (1.61 g, 14.0 mmol), EDCI (2.15 g, 11.2 mmol) and DMAP (5.00 mg, 40.9 μmol). The reaction was stirred for 1 hour at RT and concentrated in vacuo. The residue was purified by preparative HPLC to give Intermediate 10 (3.15 g, 90% purity, 72% yield) as a colorless oil. LC-MS (Method 2): Rₜ=1.38 min; MS (ESIpos): m/z=419 [M+H]⁺.

### Example S11: (4S)-4,11-diethyl-3,14-dioxo-3,4,12,14-tetrahydro-1H-pyrano[3′,4′:6,7]indolizino[1,2-b]quinolin-4-yl N-{18-[(2,5-dioxopyrrolidin-1-yl)oxy]-14,18-dioxo-4,7,10-trioxa-13-azaoctadecanan-1-oyl}-L-alpha-aspartyl-L-prolyl-L-valinate (Intermediate 11)

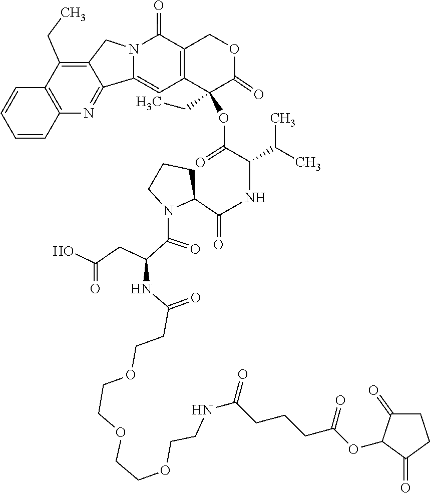 [SMILES: CCc1c2c(nc3ccccc13)-c1cc3c(c(=O)n1C2)COC(=O)[C@@]3(CC)OC(=O)[C@@H](NC(=O)[C@@H]1CCCN1C(=O)[C@H](CC(=O)O)NC(=O)CCOCCOCCOCCNC(=O)CCCC(=O)OC1C(=O)CCC1=O)C(C)C]

To a solution of (4S)-4,11-diethyl-3,14-dioxo-3,4,12,14-tetrahydro-1H-pyrano[3′,4′:6,7]indolizino[1,2-b]quinolin-4-yl N-(3-{2-[2-(2-aminoethoxy)ethoxy]ethoxy}propanoyl)-L-alpha-aspartyl-L-prolyl-L-valinate.trifluoroacetic acid (1/1) (123 mg, 123 μmol) (Intermediate 6) in DMF (10 ml) were added 1,1′-[(1,5-dioxopentane-1,5-diyl)bis(oxy)]di(pyrrolidine-2,5-dione) (120 mg, 368 μmol) and DIEA (64 μl, 370 μmol). The mixture was stirred at rt for 30 min and then concentrated under reduced pressure. The residue was purified by preparative HPLC to afford Intermediate 11 (82.0 mg, 98% purity, 59% yield) as a colorless foam. LC-MS (Method 3): Rₜ=3.65 min; MS (ESIpos): m/z=1102 [M+H]⁺.

### Example S12: Preparation of bis(2,5-dioxopyrrolidin-1-yl) N²,N⁶-bis(tert-butoxycarbonyl)-L-lysyl-L-glutamate (Intermediate 12)

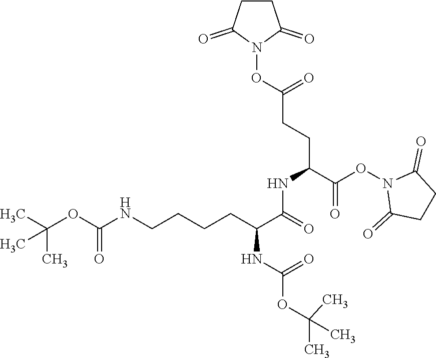 [SMILES: CC(C)(C)OC(=O)NCCCC[C@H](NC(=O)OC(C)(C)C)C(=O)N[C@@H](CCC(=O)ON1C(=O)CCC1=O)C(=O)ON1C(=O)CCC1=O]

To a solution of N²,N⁶-bis(tert-butoxycarbonyl)-L-lysyl-L-glutamic acid (50.0 mg, 82% purity, 86.3 μmol, CAS Nr=1975207-44-3) in DMF (10 ml) were added 1-hydroxypyrrolidine-2,5-dione (149 mg, 1.29 mmol), HATU (164 mg, 432 μmol) and DIEA (75 μl, 430 μmol). The mixture was stirred at rt for 1 h and then concentrated under reduced pressure. The residue was purified by preparative HPLC and then lyophilized to afford Intermediate 12 (20.5 mg, 90% purity, 32% yield) as an amorphous residue. LC-MS (Method 4): Rt=1.79 min; MS (ESIpos): m/z=670 [M+H]+

### Example S13: Preparation of (3S,19S,37S)-19-{[(2S)-2,6-diaminohexanoyl]amino}-3,37-bis[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-5,18,22,35-tetraoxo-8,11,14,26,29,32-hexaoxa-4,17,23,36-tetraazanonatriacontane-1,39-dioic acid-trifluoroacetic acid (1/2) (Intermediate 13)

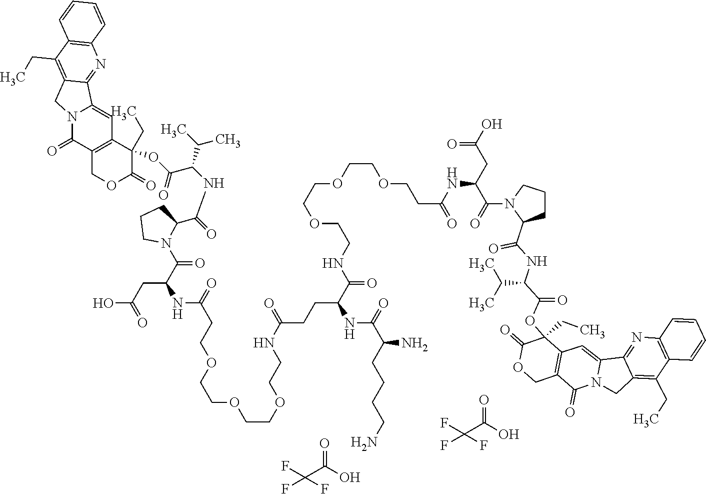 [SMILES: CCc1c2c(nc3ccccc13)-c1cc3c(c(=O)n1C2)COC(=O)[C@@]3(CC)OC(=O)[C@@H](NC(=O)[C@@H]1CCCN1C(=O)[C@H](CC(=O)O)NC(=O)CCOCCOCCOCCNC(=O)CC[C@H](NC(=O)[C@@H](N)CCCCN)C(=O)NCCOCCOCCOCCC(=O)N[C@@H](CC(=O)O)C(=O)N1CCC[C@H]1C(=O)N[C@H](C(=O)O[C@]1(CC)C(=O)OCc2c1cc1n(c2=O)Cc2c-1nc1ccccc1c2CC)C(C)C)C(C)C ; O=C(O)C(F)(F)F ; O=C(O)C(F)(F)F]

Step 1: To a solution of bis(2,5-dioxopyrrolidin-1-yl) N2,N6-bis(tert-butoxycarbonyl)-L-lysyl-L-glutamate (20.0 mg, 90% purity, 27.0 μmol) (Intermediate 12) in DMF (10 ml) were added (4S)-4,11-diethyl-3,14-dioxo-3,4,12,14-tetrahydro-1H-pyrano[3′,4′:6,7]indolizino[1,2-b]quinolin-4-yl N-(3-{2-[2-(2-aminoethoxy)ethoxy]ethoxy}propanoyl)-L-alpha-aspartyl-L-prolyl-L-valinate-trifluoroacetic acid (1/1) (57.0 mg, 56.7 μmol) (Intermediate 6) and DIEA (19 μl, 110 μmol). The mixture was stirred at rt for 5h. (4S)-4,11-Diethyl-3,14-dioxo-3,4,12,14-tetrahydro-1H-pyrano[3′,4′:6,7]indolizino[1,2-b]quinolin-4-yl N-(3-{2-[2-(2-aminoethoxy)ethoxy]ethoxy}propanoyl)-L-alpha-aspartyl-L-prolyl-L-valinate-trifluoroacetic acid (1/1) (41.0 mg) (Intermediate 6) was then added and the mixture was stirred for 72 h at rt and then concentrated under reduced pressure. The residue was purified by preparative HPLC and then lyophilized to afford (3S,19S,37S)-19-({(2S)-2,6-bis[(tert-butoxycarbonyl)amino]hexanoyl}amino)-3,37-bis[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-5,18,22,35-tetraoxo-8,11,14,26,29,32-hexaoxa-4,17,23,36-tetraazanonatriacontane-1,39-dioic acid (51 mg, 93% purity, 79% yield) as a yellow amorphous residue. LC-MS (Method 3): Rt=4.91 min; MS (ESIpos): m/z=1111 [M+2H]²⁺.

Step 2: To a solution of ((3S,19S,37S)-19-({(2S)-2,6-bis[(tert-butoxycarbonyl)amino]hexanoyl}amino)-3,37-bis[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-5,18,22,35-tetraoxo-8,11,14,26,29,32-hexaoxa-4,17,23,36-tetraazanonatriacontane-1,39-dioic acid (51.0 mg, 93% purity, 21.3 μmol) in DCM (10 ml), was added TFA (2.0 ml). The mixture was stirred at rt for 30 min and then concentrated under reduced pressure. The residue was dissolved in ACN/H₂O and lyophilized to afford Intermediate 13 (50 mg, 92% purity, 96% yield) as a yellow amorphous residue. LC-MS (Method 3): Rt=3.28 min; MS (ESIpos): m/z=1011 [M+2H]²⁺.

### Example S14: Preparation of (3S,19S,37S)-19-{[(2S)-19-amino-2-(3-{2-[2-(2-aminoethoxy)ethoxy]ethoxy}propanamido)-8-oxo-11,14,17-trioxa-7-azanonadecanan-1-oyl]amino}-3,37-bis[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-5,18,22,35-tetraoxo-8,11,14,26,29,32-hexaoxa-4,17,23,36-tetraazanonatriacontane-1,39-dioic acid-trifluoroacetic acid (1/2) (Intermediate 14)

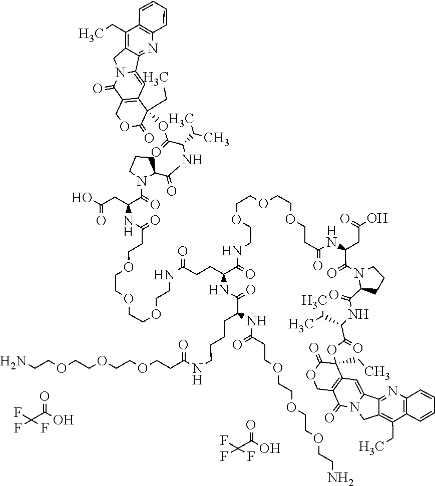 [SMILES: O=C(O)C(F)(F)F ; CCc1c2c(nc3ccccc13)-c1cc3c(c(=O)n1C2)COC(=O)[C@@]3(CC)OC(=O)[C@@H](NC(=O)[C@@H]1CCCN1C(=O)[C@H](CC(=O)O)NC(=O)CCOCCOCCOCCNC(=O)CC[C@H](NC(=O)[C@H](CCCCNC(=O)CCOCCOCCOCCN)NC(=O)CCOCCOCCOCCN)C(=O)NCCOCCOCCOCCC(=O)N[C@@H](CC(=O)O)C(=O)N1CCC[C@H]1C(=O)N[C@H](C(=O)O[C@]1(CC)C(=O)OCc2c1cc1n(c2=O)Cc2c-1nc1ccccc1c2CC)C(C)C)C(C)C ; O=C(O)C(F)(F)F]

Step 1: To a solution of (3S,19S,37S)-19-{[(2S)-2,6-diaminohexanoyl]amino}-3,37-bis[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-5,18,22,35-tetraoxo-8,11,14,26,29,32-hexaoxa-4,17,23,36-tetraazanonatriacontane-1,39-dioic acid-trifluoroacetic acid (1/2) (50.0 mg, 92% purity, 20.5 μmol) (Intermediate 13) in DME (10 ml) were added tert-butyl {2-[2-(2-{3-[(2,5-dioxopyrrolidin-1-yl)oxy]-3-oxopropoxy}ethoxy)ethoxy]ethyl}carbamate (Intermediate 10) (22.9 mg, 90% purity, 49.1 μmol) and DIEA (18 μl, 100 μmol). The mixture was stirred overnight at rt and then concentrated under reduced pressure. The residue was purified by preparative HPLC and then lyophilized to afford (3S,19S,37S)-3,37-bis[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1l-carbonyl]-19-(((23 S)-23-[(2,2-dimethyl-4,17-dioxo-3,8,11,14-tetraoxa-5-azaheptadecan-17-yl)amino]-2,2-dimethyl-4,17,24-trioxo-3,8,11,14-tetraoxa-5,18-diazatetracosan-24-yl)amino)-5,18,22,35-tetraoxo-8,11,14,26,29,32-hexaoxa-4,17,23,36-tetraazanonatriacontane-1,39-dioic acid (36 mg, 100% purity, 67% yield) as a yellow amorphous residue. LC-MS (Method 3): Rt=4.84 min; MS (ESIpos): m/z=1315 [M+2H]²⁺.

Step 2: To a solution of (3S,19S,37S)-3,37-bis[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-19-({(23S)-23-[(2,2-dimethyl-4,17-dioxo-3,8,11,14-tetraoxa-5-azaheptadecan-17-yl)amino]-2,2-dimethyl-4,17,24-trioxo-3,8,11,14-tetraoxa-5,18-diazatetracosan-24-yl}amino)-5,18,22,35-tetraoxo-8,11,14,26,29,32-hexaoxa-4,17,23,36-tetraazanonatriacontane-1,39-dioic acid (36.0 mg, 13.7 μmol) in DCM (10 ml), was added TFA (1.5 ml). The mixture was stirred at rt for 30 min and then concentrated under reduced pressure. The residue was dissolved in ACN/H₂O and lyophilized to afford Intermediate 14 (36 mg, 100% purity, 99% yield) as a yellow amorphous residue. LC-MS (Method 3): Rt=3.24 min; MS (ESIpos): m/z=810 [M+3H]³⁺

### Example S15: Preparation of tert-butyl [2-(2-{2-[2-(L-lysylamino)ethoxy]ethoxy}ethoxy)ethyl]carbamate (Intermediate 15)

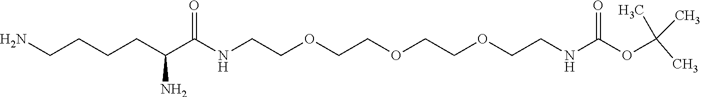 [SMILES: CC(C)(C)OC(=O)NCCOCCOCCOCCNC(=O)[C@@H](N)CCCCN]

Step 1: To a solution of N²,N⁶-bis[(benzyloxy)carbonyl]-L-lysine (50.0 mg, 121 μmol) in DMF (10 ml), were added tert-butyl (2-{2-[2-(2-aminoethoxy)ethoxy]ethoxy}ethyl)carbamate (38.8 mg, 133 μmol), HATU (59.6 mg, 157 μmol) and N,N-diisopropylethylamine (84 μl, 480 μmol). The mixture was stirred at rt for 10 min and then concentrated under reduced pressure. The residue was purified by preparative HPLC to afford benzyl [(19S)-19-{[(benzyloxy)carbonyl]amino}-2,2-dimethyl-4,18-dioxo-3,8,11,14-tetraoxa-5,17-diazatricosan-23-yl]carbamate (73 mg, 100% purity, 88% yield) as a resinous residue. LC-MS (Method 4): Rt=2.04 min; MS (ESIpos): m/z=689 [M+H]⁺

Step 2: Benzyl [(19S)-19-{[(benzyloxy)carbonyl]amino}-2,2-dimethyl-4,18-dioxo-3,8,11,14-tetraoxa-5,17-diazatricosan-23-yl]carbamate (73.0 mg, 106 μmol) was dissolved in methanol (20 ml) and dichloromethane (5 ml). Pd/C 10% (10.0 mg) was added and the reaction was hydrogenated at RT for 1 h and filtered. The mother liquor was concentrated under reduced pressure and dried under high vacuum to afford Intermediate 15 (40 mg, 100% purity, 90% yield) as an amorphous residue. LC-MS (Method 2): Rt=0.48 min; MS (ESIpos): m/z=421 [M+H]⁺.

### Example S16: Preparation of (3S,24S,49S)-24-[(2-(2-[2-(2-aminoethoxy)ethoxy]ethoxy)ethyl)carbamoyl]-3,49-bis[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-5,18,22,30,34,47-hexaoxo-8,11,14,38,41,44-hexaoxa-4,17,23,29,35,48-hexaazahenpentacontane-1,51-dioic acid-trifluoroacetic acid (1/1) (Intermediate 16)

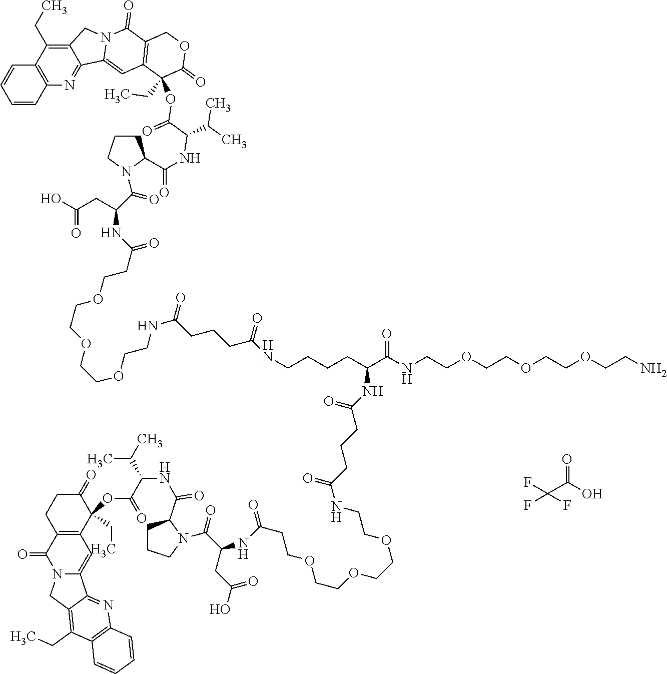 [SMILES: CCc1c2c(nc3ccccc13)-c1cc3c(c(=O)n1C2)CCC(=O)[C@@]3(CC)OC(=O)[C@@H](NC(=O)[C@@H]1CCCN1C(=O)[C@H](CC(=O)O)NC(=O)CCOCCOCCOCCNC(=O)CCCC(=O)N[C@@H](CCCCNC(=O)CCCC(=O)NCCOCCOCCOCCC(=O)N[C@@H](CC(=O)O)C(=O)N1CCC[C@H]1C(=O)N[C@H](C(=O)O[C@]1(CC)C(=O)OCc2c1cc1n(c2=O)Cc2c-1nc1ccccc1c2CC)C(C)C)C(=O)NCCOCCOCCOCCN)C(C)C ; O=C(O)C(F)(F)F]

Step 1: To a solution of tert-butyl [2-(2-{2-[2-(L-lysylamino)ethoxy]ethoxy}ethoxy)ethyl]carbamate (7.30 mg, 17.4 mol) (Intermediate 15) in DMF (10 ml) were added (4S)-4,11-diethyl-3,14-dioxo-3,4,12,14-tetrahydro-1H-pyrano[3′,4′:6,7]indolizino[1,2-b]quinolin-4-yl N-{18-[(2,5-dioxopyrrolidin-1-yl)oxy]-14,18-dioxo-4,7,10-trioxa-13-azaoctadecanan-1-oyl}-L-alpha-aspartyl-L-prolyl-L-valinate (41.0 mg, 98% purity, 36.5 μmol) (Intermediate 11) and DIEA (12 μl, 69 μmol). The mixture was stirred overnight at rt and then concentrated under reduced pressure. The residue was purified by preparative HPLC and then lyophilized to afford (3S,24S,49S)-3,49-bis[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-24-[(2,2-dimethyl-4-oxo-3,8,11,14-tetraoxa-5-azahexadecan-16-yl)carbamoyl]-5,18,22,30,34,47-hexaoxo-8,11,14,38,41,44-hexaoxa-4,17,23,29,35,48-hexaazahenpentacontane-1,51-dioic acid (27 mg, 98% purity, 63% yield) as an amorphous residue. LC-MS (Method 3): Rt=4.46 min; MS (ESIpos): m/z=1198 [M+2H]²⁺.

Step 2: To a solution of (3S,24S,49S)-3,49-bis[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-24-[(2,2-dimethyl-4-oxo-3,8,11,14-tetraoxa-5-azahexadecan-16-yl)carbamoyl]-5,18,22,30,34,47-hexaoxo-8,11,14,38,41,44-hexaoxa-4,17,23,29,35,48-hexaazahenpentacontane-1,51-dioic acid (27.0 mg, 98% purity, 11.0 μmol) in DCM (10 ml), was added TFA (1.0 ml). The mixture was stirred at rt for 30 min and then concentrated under reduced pressure. The residue was dissolved in ACN/H₂O and lyophilized to afford Intermediate 16 (28 mg, 92% purity, 98% yield) as a yellow amorphous residue. LC-MS (Method 3): Rt=3.59 min; MS (ESIpos): m/z=1147 [M+2H]²⁺.

### Example S17: Preparation of (3S,19S,37S)-19-amino-3,37-bis[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-5,18,22,35-tetraoxo-8,11,14,26,29,32-hexaoxa-4,17,23,36-tetraazanonatriacontane-1,39-dioic acid-trifluoroacetic acid (1/1) (Intermediate 17)

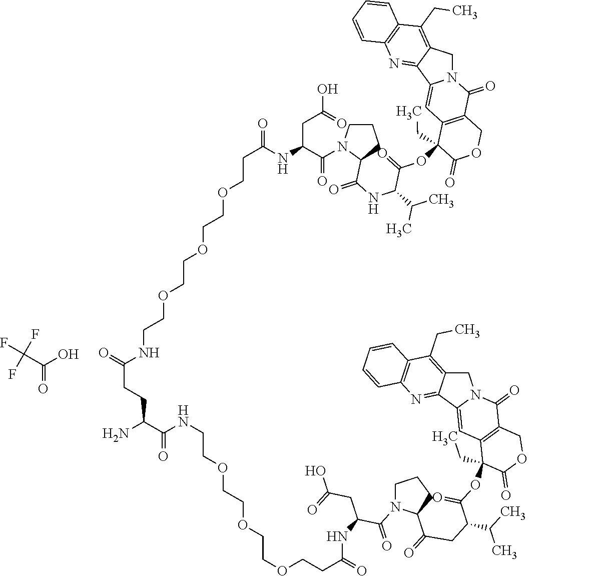 [SMILES: O=C(O)C(F)(F)F ; CCc1c2c(nc3ccccc13)-c1cc3c(c(=O)n1C2)COC(=O)[C@@]3(CC)OC(=O)[C@@H](CC(=O)[C@@H]1CCCN1C(=O)[C@H](CC(=O)O)NC(=O)CCOCCOCCOCCNC(=O)[C@@H](N)CCC(=O)NCCOCCOCCOCCC(=O)N[C@@H](CC(=O)O)C(=O)N1CCC[C@H]1C(=O)N[C@H](C(=O)O[C@]1(CC)C(=O)OCc2c1cc1n(c2=O)Cc2c-1nc1ccccc1c2CC)C(C)C)C(C)C]

Step 1: To a solution of bis(2,5-dioxopyrrolidin-1-yl)N-(tert-butoxycarbonyl)-L-glutamate (6.59 mg, 14.9 μmol, CAS Nr=246234-73-1) in DMF (1.5 ml) were added (4S)-4,11-diethyl-3,14-dioxo-3,4,12,14-tetrahydro-1H-pyrano[3′,4′:6,7]indolizino[1,2-b]quinolin-4-yl N-(3-{2-[2-(2-aminoethoxy)ethoxy]ethoxy}propanoyl)-L-alpha-aspartyl-L-prolyl-L-valinate-trifluoroacetic acid (1/1) (30.0 mg, 29.9 μmol) (Intermediate 6) and DIEA (16 μl, 90 μmol). The mixture was stirred at rt for 1 h and then concentrated under reduced pressure. The residue was purified by preparative HPLC and then lyophilized to afford (3S,19S,37S)-19-[(tert-butoxycarbonyl)amino]-3,37-bis[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-5,18,22,35-tetraoxo-8,11,14,26,29,32-hexaoxa-4,17,23,36-tetraazanonatriacontane-1,39-dioic acid (16.5 mg, 100% purity, 55% yield) as an amorphous residue. LC-MS (Method 3): Rt=4.53 min; MS (ESIpos): m/z=997 [M+2H]²⁺

Step 2: To a solution of (3S,19S,37S)-19-[(tert-butoxycarbonyl)amino]-3,37-bis[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-5,18,22,35-tetraoxo-8,11,14,26,29,32-hexaoxa-4,17,23,36-tetraazanonatriacontane-1,39-dioic acid (16.0 mg, 100% purity, 8.03 μmol) in DCM (1.5 ml), was added TFA (0.75 ml). The mixture was stirred at rt for 1 h and then concentrated under reduced pressure. The residue was dissolved in ACN/H₂O and lyophilized to afford Intermediate 17 (16.1 mg, 100% purity, 100% yield) as an amorphous residue. LC-MS (Method 3): Rt=3.71 min; MS (ESIpos): m/z=1893 [M+H]⁺.

### Example S18: Preparation of (3S,19S,37S)-19-(3-{2-[2-(2-aminoethoxy)ethoxy]ethoxy}propanamido)-3,37-bis[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-5,18,22,35-tetraoxo-8,11,14,26,29,32-hexaoxa-4,17,23,36-tetraazanonatriacontane-1,39-dioic acid-trifluoroacetic acid (1/1) (Intermediate 18)

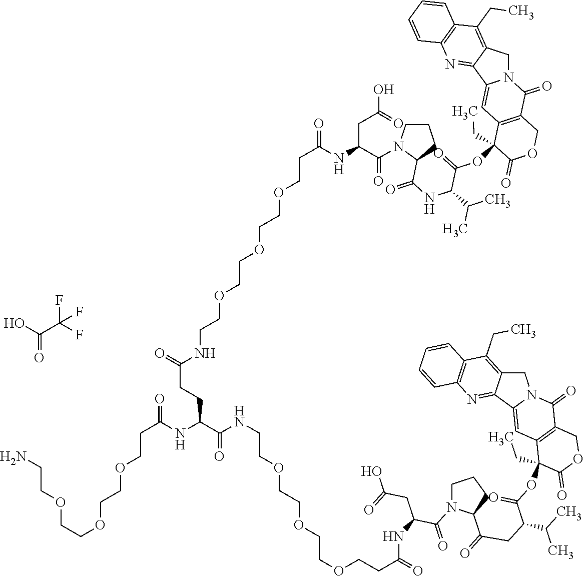 [SMILES: O=C(O)C(F)(F)F ; CCc1c2c(nc3ccccc13)-c1cc3c(c(=O)n1C2)COC(=O)[C@@]3(CC)OC(=O)[C@@H](CC(=O)[C@@H]1CCCN1C(=O)[C@H](CC(=O)O)NC(=O)CCOCCOCCOCCNC(=O)[C@H](CCC(=O)NCCOCCOCCOCCC(=O)N[C@@H](CC(=O)O)C(=O)N1CCC[C@H]1C(=O)N[C@H](C(=O)O[C@]1(CC)C(=O)OCc2c1cc1n(c2=O)Cc2c-1nc1ccccc1c2CC)C(C)C)NC(=O)CCOCCOCCOCCN)C(C)C]

Step 1: Toa solution of(3S,19S,37S)-19-amino-3,37-bis[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-5,18,22,35-tetraoxo-8,11,14,26,29,32-hexaoxa-4,17,23,36-tetraazanonatriacontane-1,39-dioic acid-trifluoroacetic acid (1/1) (16.0 mg, 100% purity, 7.97 μmol) (Intermediate 17) in DMF (1.6 ml) were added tert-butyl {2-[2-(2-{3-[(2,5-dioxopyrrolidin-1-yl)oxy]-3-oxopropoxy}ethoxy)ethoxy]ethyl}carbamate (Intermediate 10) (4.00 mg, 9.57 μmol) and DIEA (5.6 μl, 32 μmol). The mixture was stirred at rt for 3 h and then concentrated under reduced pressure. The residue was purified by preparative HPLC and then dried under high vacuum to afford (3S,19S,37S)-3,37-bis[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-19-[(2,2-dimethyl-4,17-dioxo-3,8,11,14-tetraoxa-5-azaheptadecan-17-yl)amino]-5,18,22,35-tetraoxo-8,11,14,26,29,32-hexaoxa-4,17,23,36-tetraazanonatriacontane-1,39-dioic acid (12.8 mg, 100% purity, 73% yield). LC-MS (Method 3): Rt=4.59 min; MS (ESIpos): m/z=1098 [M+2H]²⁺.

Step 2: To a solution of (3S,19S,37S)-3,37-bis[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-19-[(2,2-dimethyl-4,17-dioxo-3,8,11,14-tetraoxa-5-azaheptadecan-17-yl)amino]-5,18,22,35-tetraoxo-8,11,14,26,29,32-hexaoxa-4,17,23,36-tetraazanonatriacontane-1,39-dioic acid (12.6 mg, 5.74 μmol) in DCM (2 ml), was added TFA (0.5 ml). The mixture was stirred at rt for 1 h and then concentrated under reduced pressure. The residue was dissolved in ACN/H₂O and lyophilized to afford Intermediate 18 (12.1 mg, 100% purity, 95% yield) as an amorphous residue.

LC-MS (Method 3): Rt=3.65 min; MS (ESIpos): m/z=1048 [M+2H]²⁺

### Example S19: Preparation of pentafluorophenyl 4-oxo-4-[(5-sulfamoyl-1,3,4-thiadiazol-2-yl)amino]butanoate (Intermediate 19)

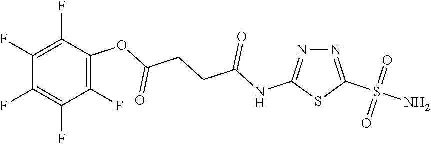 [SMILES: NS(=O)(=O)c1nnc(NC(=O)CCC(=O)Oc2c(F)c(F)c(F)c(F)c2F)s1]

To a solution of 4-oxo-4-[(5-sulfamoyl-1,3,4-thiadiazol-2-yl)amino]butanoic acid (100 mg, 357 μmol, CAS Nr: 78851-85-1) in pyridine (100 ml) were added pentafluorophenol (131 mg, 714 μmol) and EDCI (137 mg, 714 μmol; CAS Nr: 25952-53-8). The mixture was stirred at rt for 20 h and then concentrated under reduced pressure. The residue was dissolved in ethyl acetate and water and the two layers were separated. The organic phase was washed with water, dried over magnesium sulphate and concentrated under reduced pressure to afford Intermediate 19 (36 mg, 23% yield) which was used in the next step without further purification.

### Example S20: Preparation of 3-[[6-chloro-4-(methylsulfanylmethyl)-2-pyridyl]oxy]propan-1-ol (Intermediate 20)

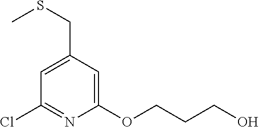

To a solution of propane-1,3-diol (10.97 g, 144.16 mmol, 10.45 mL, 2.5 eq) in THF (144 mL) was added NaH (3.00 g, 74.96 mmol, 60% purity, 1.3 eq) at 0° C. under N₂. The mixture was stirred at 20° C. for 30 min, then 2,6-dichloro-4-(methylsulfanylmethyl)pyridine (12 g, 57.66 mmol, 1 eq) was added to the reaction mixture at 20° C. under N₂. The mixture was stirred at 70° C. for 15.5 h. The reaction mixture was poured into NH₄Cl (100 mL). The aqueous phase was extracted with ethyl acetate (3×200 mL). The combined organic phase was washed with brine (2×200 mL), dried with anhydrous Na₂SO₄, filtered, and concentrated in vacuo. The crude product was purified by column chromatography (SiO₂, petroleum ether/ethyl acetate=26:1 to 10:1) to give Intermediate 20 (11 g, 44.40 mmol, 77% yield) as a colorless oil. ¹H NMR (400 MHz, CDC₃) δ 6.90 (s, 1H), 6.59 (d, J=1.0 Hz, 1H), 4.48 (t, J=6.0 Hz, 2H), 3.75 (t, J=5.8 Hz, 2H), 3.54 (s, 2H), 2.45 (br s, 1H), 2.03-1.95 (m, 5H).

### Example S21: Preparation of 4-[2-[3-[[6-chloro-4-(methylsulfanylmethyl)-2-pyridyl]oxy]propoxy]-4-fluoro-phenyl]-5-fluoro-pyridin-2-amine (Intermediate 21)

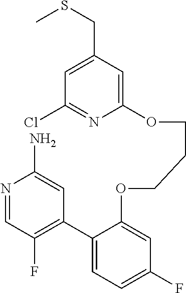 [SMILES: CSCc1cc(Cl)nc(OCCCOc2cc(F)ccc2-c2cc(N)ncc2F)c1]

To a mixture of 3-[[6-chloro-4-(methylsulfanylmethyl)-2-pyridyl]oxy]propan-1-ol (10.43 g, 42.08 mmol, 1.1 eq) and 2-(2-amino-5-fluoro-4-pyridyl)-5-fluoro-phenol (8.5 g, 38.26 mmol, 1 eq) in toluene (100 mL) was added CMBP (27.70 g, 114.77 mmol, 3 eq) at 20° C. under N₂. The mixture was stirred at 110° C. for 16 hours. The reaction mixture was poured into water (40 mL). The aqueous phase was extracted with ethyl acetate (3×50 mL). The combined organic phase was washed with brine (2×50 mL), dried with anhydrous Na₂SO₄, filtered, and concentrated in vacuo. The crude product was purified by column chromatography (SiO₂, petroleum ether/ethyl acetate=20:1 to 8:1) to give Intermediate 21(16.7 g, 25.87 mmol, 68% yield, 70% purity) as a brown oil. ¹H NMR (400 MHz, CDCl₃-d) δ 7.94 (d, J=2.0 Hz, 1H), 7.21 (dd, J=6.6, 8.8 Hz, 1H), 6.88 (s, 1H), 6.77-6.64 (m, 2H), 6.55 (s, 1H), 6.52-6.43 (m, 1H), 4.46 (s, 2H), 4.42-4.30 (m, 2H), 4.16-4.06 (m, 2H), 3.52 (s, 2H), 2.16 (quin, J=6.0 Hz, 2H), 1.98 (s, 3H).

### Example S22: Preparation of 5,22-difluoro-15-(methylsulfanylmethyl)-8,12-dioxa-18,20,24-triazatetracyclo[17.3.1.1\hat{ }{13,17}.0\hat{ }{2,7}]tetracosa-1(22),2,4,6,13,15,17(24),19(23),20-nonaene (Intermediate 22)

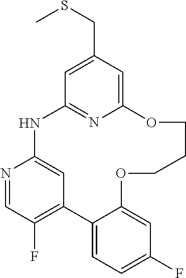 [SMILES: CSCc1cc2nc(c1)OCCCOc1cc(F)ccc1-c1cc(ncc1F)N2]

To a mixture of Intermediate 21(10 g, 15.49 mmol, 70% purity, 1 eq) in toluene (100 mL) and NMP (20 mL) was added K₃PO₄ (16.44 g, 77.45 mmol, 5 eq), XPhos (738.41 mg, 1.55 mmol, 0.1 eq) and Xphos Pd G1 (572.16 mg, 774.48 umol, 0.05 eq) at 20° C. under N₂. The mixture was stirred at 110° C. for 3 hours. The reaction mixture was poured into water (50 mL). The aqueous phase was extracted with ethyl acetate (3×80 mL). The combined organic phase was washed with brine (2×100 mL), dried with anhydrous Na₂SO₄, filtered, and concentrated in vacuo. The crude product was purified by column chromatography (SiO₂, petroleum ether:ethyl acetate=20:1 to 5:1) to give Intermediate 22 (5 g, 12.03 mmol, 77.70% yield) as a light-yellow solid. ¹H NMR (400 MHz, CDCl₃) δ 8.80 (d, J=6.0 Hz, 1H), 8.17 (d, J=2.8 Hz, 1H), 7.61 (ddd, J=3.8, 6.6, 8.4 Hz, 1H), 7.26 (br s, 1H), 6.89-6.56 (m, 2H), 6.22 (s, 2H), 4.68-4.55 (m, 2H), 4.12-3.99 (m, 2H), 3.53 (s, 2H), 2.32-2.18 (m, 2H), 2.05 (s, 3H).

### Example S23: Preparation of (5,22-difluoro-8,12-dioxa-18,20,24-triazatetracyclo[17.3.1.113,17.02,7]tetracosa-1(22),2,4,6,13,15,17(24),19(23),20-nonaen-15-yl)methyl-imino-methyl-oxo-λ⁶-sulfane (Intermediate 23, Racemic Mixture and Intermediate 23-a & 23-b, Single Enantiomers)

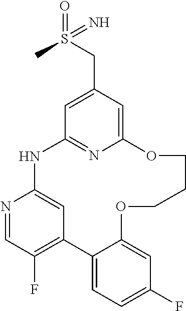 [SMILES: C[S@](=N)(=O)Cc1cc2nc(c1)OCCCOc1cc(F)ccc1-c1cc(ncc1F)N2]

To a mixture of Intermediate 22 (400 mg, 962.80 umol, 1 eq) in DCM (10 mL) was added ammonia:carbamic acid (751.66 mg, 9.63 mmol, 10 eq) and PhI(OAc)₂ (775.28 mg, 2.41 mmol, 2.5 eq) at 20° C. under N₂. The mixture was stirred at 20° C. for 16 hours. 12 reactions were conducted in parallel. The reaction mixture was poured into water (50 mL). The aqueous phase was extracted with DCM (3×80 mL). The combined organic phase was washed with brine (2×80 mL), dried with anhydrous Na₂SO₄, filtered, and concentrated in vacuo. The reaction mixture was purified by column chromatography (SiO₂, petroleum ether/ethyl acetate=1:1 to ethyl acetate:MeOH=50:1) to give Intermediate 23 (2.1 g, 4.47 mmol, 38.6% yield, 95% purity) as a light-yellow solid. ¹H NMR (400 MHz, DMSO-4) δ 9.71 (s, 1H), 8.69 (s, 1H), 8.31 (d, J=2.4 Hz, 1H), 7.57 (br s, 1H), 7.08 (dd, J=2.2, 11.4 Hz, 1H), 6.90 (dt, J=2.4, 8.4 Hz, 1H), 6.58 (s, 1H), 6.26 (s, 1H), 4.61-4.41 (m, 2H), 4.34-4.22 (m, 2H), 4.19-4.05 (m, 2H), 3.73 (s, 1H), 2.87 (s, 3H), 2.10 (br d, J=6.0 Hz, 2H).

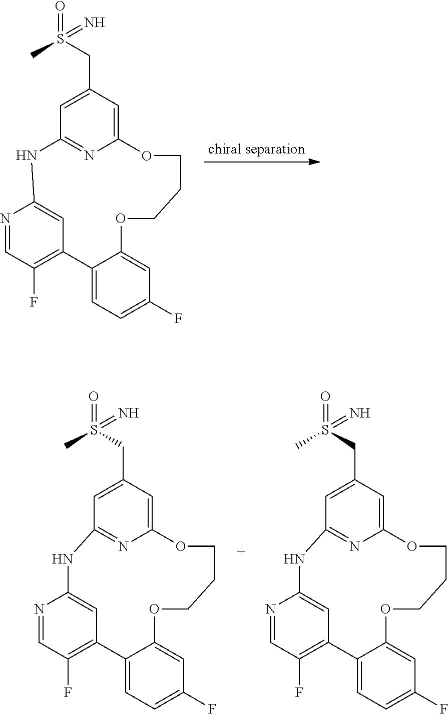 [SMILES: C[S@](=N)(=O)Cc1cc2nc(c1)OCCCOc1cc(F)ccc1-c1cc(ncc1F)N2 ; C[S@](=N)(=O)Cc1cc2nc(c1)OCCCOc1cc(F)ccc1-c1cc(ncc1F)N2 ; C[S@@](=N)(=O)Cc1cc2nc(c1)OCCCOc1cc(F)ccc1-c1cc(ncc1F)N2]

Intermediate 23 was separated by prep-SFC(column: DAICEL CHIRALCEL OJ (250 mm*50 mm, 10 um); mobile phase: [0.1% NH₃H₂O ETOH]; B %: 60%-60%,14 min, Rₜ=1.67, Rₜ2=2.183) to give Intermediate 23-a (750 mg, 1.52 mmol, 35.4% yield, 99.18% purity) as an off-white solid and Intermediate 23-b (810 mg, 1.62 mmol, 37.7% yield, 97.77% purity) as a light-yellow solid. Note: The R/S configuration of these 2 compounds were not confirmed.

Intermediate 23-a: ¹H NMR (400 MHz, DMSO-d₆) δ 9.71 (s, 1H), 8.69 (d, J=6.0 Hz, 1H), 8.31 (d, J=2.4 Hz, 1H), 7.57 (dt, J=2.8, 4.2 Hz, 1H), 7.07 (dd, J=2.2, 11.4 Hz, 1H), 6.89 (dt, J=2.4, 8.4 Hz, 1H), 6.58 (s, 1H), 6.26 (s, 1H), 4.59-4.42 (m, 2H), 4.28 (d, J=2.2 Hz, 2H), 4.19-4.05 (m, 2H), 3.74 (s, 1H), 2.88 (s, 3H), 2.09 (br d, J=6.2 Hz, 2H). The desired enantiomer (optical rotation −4.88°±0.00, 20C, 589 nm) was obtained with 100% ee at Rₜ 1.63-1.83 min. LC-MS: Rₜ=2.38 min; MS (ESIpos): m/z=447 [M+H]⁺. Single (−) isomer, absolute stereochemistry unknown.

Intermediate 23-b: ¹H NMR (400 MHz, DMSO-d) δ 9.70 (s, 1H), 8.68 (br d, J=5.8 Hz, 1H), 8.34-8.27 (m, 1H), 7.62-7.52 (m, 1H), 7.07 (br d, J=9.8 Hz, 1H), 6.94-6.84 (m, 1H), 6.58 (s, 1H), 6.26 (s, 1H), 4.59-4.41 (m, 2H), 4.38-4.19 (m, 2H), 4.11 (br s, 2H), 3.74 (s, 1H), 2.88 (s, 3H), 2.18-2.01 (m, 2H). The desired enantiomer (optical rotation 3.86° 0.00, 20C, 589 nm) was obtained with 100% ee at Rₜ 2.05-2.42 min. LC-MS: Rₜ=2.38 min; MS (ESIpos): m/z=447 [M+H]⁺. Single (+) isomer, absolute stereochemistry unknown.

### Example S24: Preparation of tert-butyl N-[(1S)-1-[[(5,22-difluoro-8,12-dioxa-18,20-diazatetracyclo[17.3.1.113,17.02,7]tetracosa-1(23),2(7),3,5,13(24),14,16,19,21-nonaen-15-yl)methyl-methyl-oxo-λ⁶-sulfanylidene]carbamoyl]-2-methyl-propyl]carbamate (Intermediate 24)

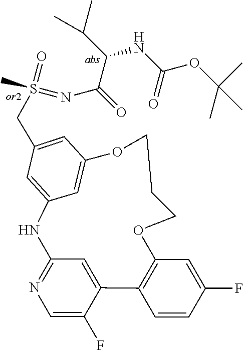 [SMILES: CC(C)[C@H](NC(=O)OC(C)(C)C)C(=O)N=[S@@](C)(=O)Cc1cc2cc(c1)OCCCOc1cc(F)ccc1-c1cc(ncc1F)N2]

(5,22-difluoro-8,12-dioxa-18,20,24-triazatetracyclo[17.3.1.113,17.02,7]tetracosa-1(22),2,4,6,13,15,17(24),19(23),20-nonaen-15-yl)methyl-imino-methyl-oxo-λ⁶-sulfane (300 mg, 671 μmol) (Intermediate 23-b) was dissolved in DMF (30 mL). N-(tert-butoxycarbonyl)-L-valine (204 mg, 940 μmol), HATU (510 mg, 1.34 mmol) and DIEA (468 μl, 2.69 mmol) were added. The reaction was stirred at RT for 16 hours and then concentrated in vacuo. The residue was dissolved in DCM and washed with a citric acid solution (5% in water) and concentrated. Water was then added, and the resulting precipitate was filtered, washed with water and dried over high vacuum to afford Intermediate 24 (400 mg, 75.4% purity, 69.5% yield). LC-MS (Method 2): Rₜ=2.42 min; MS (ESIpos): m/z=646 [M+H]⁺.

### Example S25: Preparation of Trifluoroacetic Acid. (2S)-2-amino-N-[(5,22-difluoro-8,12-dioxa-18,20-diazatetracyclo[17.3.1.113,17.02,7]tetracosa-1(23),2(7),3,5,13(24),14,16,19,21-nonaen-15-yl)methyl-methyl-oxo-λ⁶-sulfanylidene]-3-methyl-butanamide (1/1) (Intermediate 25)

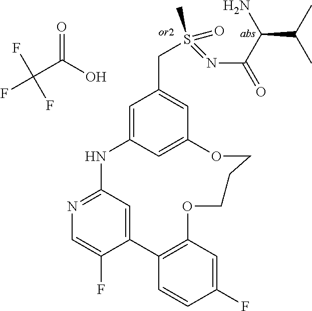 [SMILES: O=C(O)C(F)(F)F ; CC(C)[C@H](N)C(=O)N=[S@@](C)(=O)Cc1cc2cc(c1)OCCCOc1cc(F)ccc1-c1cc(ncc1F)N2]

Tert-butyl N-[(1S)-1-[[(5,22-difluoro-8,12-dioxa-18,20-diazatetracyclo[17.3.1.113,17.02,7]tetracosa-1(23),2(7),3,5,13(24),14,16,19,21-nonaen-15-yl)methyl-methyl-oxo-λ⁶-sulfanylidene]carbamoyl]-2-methyl-propyl]carbamate (400 mg, 75.4% purity, 467 μmol) (Intermediate 24) was dissolved in DCM (30 mL), then TFA (10 ml) was added. The reaction was stirred for 30 min at RT and was then concentrated in vacuo. The residue was dissolved in ACN/water and freeze-dried. The residue was purified over preparative HPLC and lyophilized to give Intermediate 25 (286 mg, 100% purity, 92.8% yield) as an amorphous residue. LC-MS (Method 2): Rₜ=1.41 min; MS (ESIpos): m/z=546 [M+H].

### Example S26: Preparation of tert-butyl (3S)-3-[3-[2-[2-[2-(tert-butoxycarbonylamino)ethoxy]ethoxy]ethoxy]propanoylamino]-4-[(2S)-2-[[(15)-1-[[(5,22-difluoro-8,12-dioxa-18,20,24-triazatetracyclo[17.3.1.113,17.02,7]tetracosa-1(23),2(7),3,5,13(24),14,16,19,21-nonaen-15-yl)methyl-methyl-oxo-4′-sulfanylidene]carbamoyl]-2-methyl-propyl]carbamoyl]pyrrolidin-1-yl]-4-oxo-butanoate (Intermediate 26)

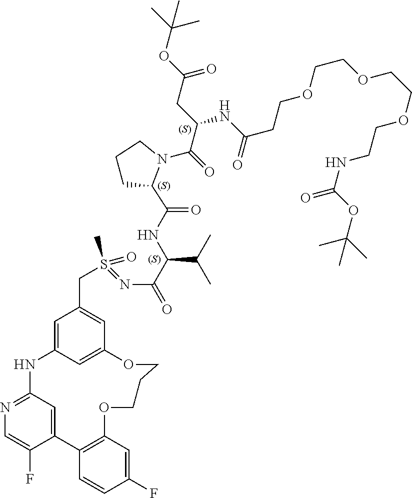

Trifluoroacetic acid. (2S)-2-amino-N-[(5,22-difluoro-8,12-dioxa-18,20-diazatetracyclo[17.3.1.113,17.02,7]tetracosa-1(23),2(7),3,5,13(24),14,16,19,21-nonaen-15-yl)methyl-methyl-oxo-λ⁶-sulfanylidene]-3-methyl-butanamide (1/1) (266 mg, 403 μmol) (Intermediate 25) was dissolved in DMF (80 ml). (2S)-1-[(19S)-19-(2-Tert-butoxy-2-oxoethyl)-2,2-dimethyl-4,17,20-trioxo-3,8,11,14-tetraoxa-5,18-diazaicosan-20-yl]pyrrolidine-2-carboxylic acid (Intermediate 3) (262 mg, 443 μmol), HATU (245 mg, 645 μmol) and DIEA (211 μl, 1.21 mmol) were added. The reaction was stirred at RT for 1 h 30 and was then concentrated in vacuo. The residue was separated by prep. HPLC to give Intermediate 26 (326 mg, 100% purity, 72.4% yield). LC-MS (Method 2): Rₜ=2.33 min; MS (ESIpos): m/z=1117 [M+H]⁺.

### Example S27: Preparation of N-(3-{2-[2-(2-aminoethoxy)ethoxy]ethoxy}propanoyl)-L-alpha-aspartyl-L-prolyl-N—[(R*)—{[15,19-difluoro-3,4-dihydro-2H,11H-10,6-(azeno)-12,16-(metheno)-1,5,11,13-benzodioxadiazacyclooctadecin-8-yl]methyl}(methyl)oxo-lambda⁶-sulfanylidene]-L-valinamide.trifluoroacetic acid (1/1) (Intermediate 27)

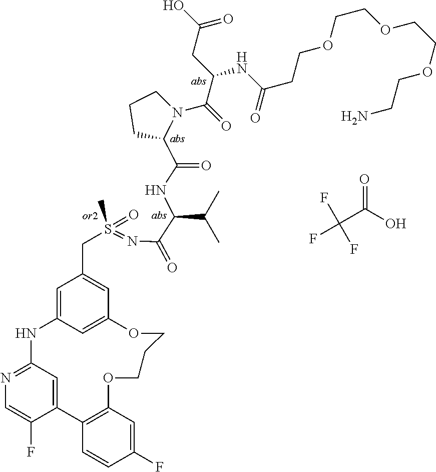 [SMILES: CC(C)[C@H](NC(=O)[C@@H]1CCCN1C(=O)[C@H](CC(=O)O)NC(=O)CCOCCOCCOCCN)C(=O)N=[S@@](C)(=O)Cc1cc2cc(c1)OCCCOc1cc(F)ccc1-c1cc(ncc1F)N2 ; O=C(O)C(F)(F)F]

Tert-butyl (3S)-3-[3-[2-[2-[2-(tert-butoxycarbonylamino)ethoxy]ethoxy]ethoxy]propanoylamino]-4-[(2S)-2-[[(15)-1-[[(5,22-difluoro-8,12-dioxa-18,20,24-triazatetracyclo[17.3.1.113,17.02,7]tetracosa-1(23),2(7),3,5,13(24),14,16,19,21-nonaen-15-yl)methyl-methyl-oxo-λ⁶-sulfanylidene]carbamoyl]-2-methyl-propyl]carbamoyl]pyrrolidin-1-yl]-4-oxo-butanoate (326 mg, 292 μmol) (Intermediate 26) was dissolved in DCM (80 ml), then TFA (20 ml) was added. The reaction was stirred for 3 hours at RT and was concentrated in vacuo. The residue was dissolved in ACN/water and freeze-dried to give Intermediate 27 (374 mg, 100% purity, quant.) as an amorphous residue. LC-MS (Method 5): Rₜ=0.78 min; MS (ESIpos): m/z=960 [M+H]⁺.

### Example S28: Preparation of Benzyl [(1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl]carbamate (Intermediate 28)

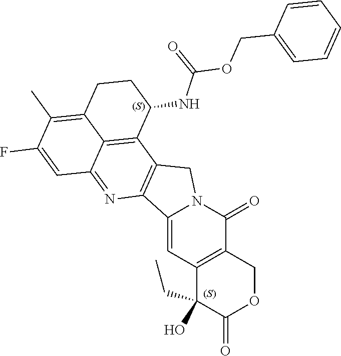

Methane sulfonic acid-(1S,9S)-1-amino-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (1/1) (exatecan mesylate) (50.0 mg, 94.1 μmol) was dissolved in DMF (10.0 mL), then 1-{[(benzyloxy)carbonyl]oxy}pyrrolidine-2,5-dione (28.1 mg, 113 μmol) and DIEA (49 μl, 280 μmol) were added. The reaction was stirred overnight at rt. The reaction was concentrated in vacuo and the residue was mixed with ACN/H₂O/DMF. A solid precipitated, which was filtered off, dried and purified by prep. HPLC to give Intermediate 28 (50.0 mg, 92% purity, 87% yield) as a white foam. LC-MS (Method 6): Rₜ=3.21 min; MS (ESIpos): m/z=570 [M+H]⁺.

### Example S29: Preparation of (1S,9S)-1-{[(Benzyloxy)carbonyl]amino}-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-(tert-butoxycarbonyl)-L-valinate (Intermediate 29)

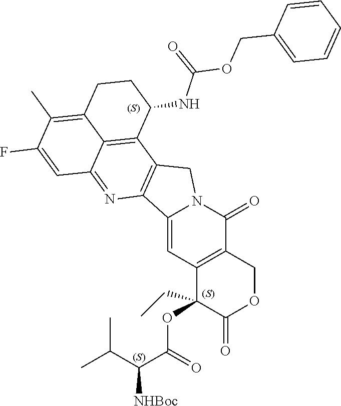

Benzyl [(1 S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl]carbamate (Intermediate 28) (50.0 mg, 87.8 μmol) was added to DCM (20.0 mL), then tert-butyl (4S)-2,5-dioxo-4-(propan-2-yl)-1,3-oxazolidine-3-carboxylate (51.2 mg, 211 μmol) and DMAP (19.3 mg, 158 μmol; CAS: 1122-58-3) were added. The mixture was refluxed while stirring for 7 h. The reaction was concentrated in vacuo. Water was added, upon which the product precipitated. The mixture was filtered, the filter residue was very poorly soluble, but was soluble in DMSO+ACN or MeOH/DCM. The compound was purified by prep. HPLC to give Intermediate 29 (54.0 mg, 98% purity, 78% yield). LC-MS (Method 2): Rₜ=2.48 min; MS (ESIpos): m/z=769 [M+H]⁺.

### Example S30: Preparation of Trifluoroacetic acid-(1S,9S)-1-{[(benzyloxy)carbonyl]amino}-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl L-valinate (1/1) (Intermediate 30)

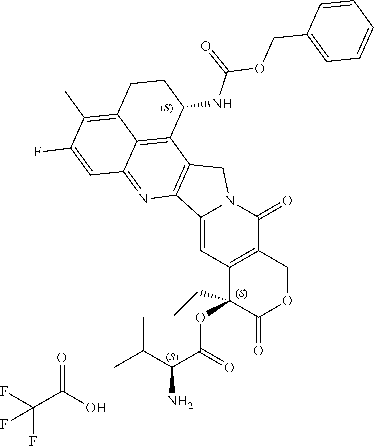

(1S,9S)-1-([(Benzyloxy)carbonyl]amino)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-(tert-butoxycarbonyl)-L-valinate (Intermediate 29) (44.0 mg, 98% purity, 55.9 μmol) was dissolved in DCM (6.3 mL), TFA (1.3 mL) was added and the reaction was stirred at rt for 30 min. The reaction was concentrated in vacuo, the residue was dissolved in ACN/H₂O and lyophilized to give Intermediate 30 (44.0 mg, 98% purity, 98% yield). LC-MS (Method 6): R, =2.56 min; MS (ESIpos): m/z=669 [M+H]⁺.

### Example S31: Preparation of Tert-butyl (19S)-19-{[(2S)-2-{[(2S)-1-{[(1S,9S)-1-{[(benzyloxy)carbonyl]amino}-9-ethyl-5-fluoro-4-methyl-10,13-dioxo-2,3,9,10,13,15-hexahydro-1H,12H-benzo[de]pyrano [3′,4′:6,7]indolizino [1,2-b]quinolin-9-yl]oxy}-3-methyl-1-oxobutan-2-yl]carbamoyl}pyrrolidine-1-carbonyl]-2,2-dimethyl-4,17-dioxo-3,8,11,14-tetraoxa-5,18-diazahenicosan-21-oate (Intermediate 31)

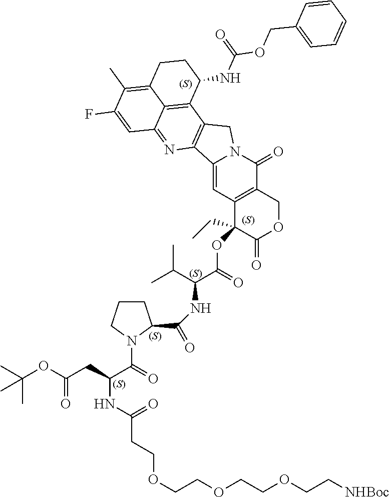

To a solution of trifluoroacetic acid-(1S,9S)-1-{[(benzyloxy)carbonyl]amino}-9-ethyl-5-fluoro-4-methyl-10,13-dioxo-2,3,9,10,13,15-hexahydro-1H,12H-benzo[de]pyrano [3′,4′:6,7]indolizino [1,2-b]quinolin-9-yl L-valinate (1/1) (44.0 mg, 98% purity, 54.9 μmol) (Intermediate 30) and (2S)-1-[(19S)-19-(2-tert-butoxy-2-oxoethyl)-2,2-dimethyl-4,17,20-trioxo-3,8,11,14-tetraoxa-5,18-diazaicosan-20-yl]pyrrolidine-2-carboxylic acid (Intermediate 3) (38.9 mg, 65.9 μmol) in DMF (8.0 mL) were added HATU ((27.1 mg, 71.4 μmol) and DIEA (29 μl, 160 μmol). The solution was stirred at rt for 30 min and then concentrated in vacuo. The residue was purified by prep. HPLC, then lyophilized to give the Intermediate 31 (50.0 mg, 100% purity, 73% yield) as a yellow foam. LC-MS (Method 6): R: =4.28 min; MS (ESIpos): m/z=1239 [M+H]⁺.

### Example S32: Tert-butyl (19S)-19-[(2S)-2-{[(2S)-1-{[(1S,9S)-1-amino-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl]oxy}-3-methyl-1-oxobutan-2-yl]carbamoyl}pyrrolidine-1-carbonyl]-2,2-dimethyl-4,17-dioxo-3,8,11,14-tetraoxa-5,18-diazahenicosan-21-oate (Intermediate 32)

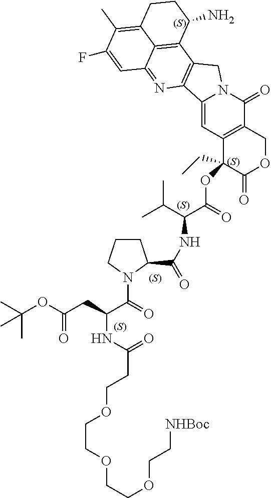

Tert-butyl (19S)-19-[(2S)-2-{[(2S)-1-{[(1S,9S)-1-{[(benzyloxy)carbonyl]amino}-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl]oxy}-3-methyl-1-oxobutan-2-yl]carbamoyl}pyrrolidine-1-carbonyl]-2,2-dimethyl-4,17-dioxo-3,8,11,14-tetraoxa-5,18-diazahenicosan-21-oate (350 mg, 282 μmol) (Intermediate 31) was dissolved in ethanol (50 ml). The catalyst Pd/C 10%(50.0 mg) was added. The reaction was hydrogenated for 2 hour at standard pressure. The catalyst was filtered off and the filtrate was concentrated on a rotary evaporator. The residue was dissolved in ACN/H2O and freeze-dried to give Intermediate 32(287 mg, 86% purity, 79% yield) as a colorless foam. LC-MS (Method 3): Rₜ=3.59 min; MS (ESIpos): m/z=1107 [M+H]⁺.

### Example S33: (1S,9S)-1-Amino-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-(3-{2-[2-(2-aminoethoxy) ethoxy]ethoxy}propanoyl)-L-alpha-aspartyl-L-prolyl-L-valinate trifluoroacetic acid (1/1) (Intermediate 33)

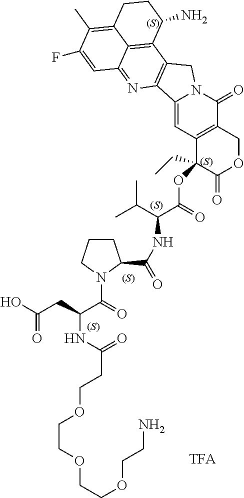

Tert-butyl (19S)-19-[(2S)-2-{[(2S)-1-{[(1S,9S)-1-amino-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl]oxy}-3-methyl-1-oxobutan-2-yl]carbamoyl}pyrrolidine-1-carbonyl]-2,2-dimethyl-4,17-dioxo-3,8,11,14-tetraoxa-5,18-diazahenicosan-21-oate (287 mg, 86% purity, 223 μmol) (Intermediate 32) was dissolved in DCM (30 ml), then TFA (5.0 ml) was added. The reaction was stirred for 1 hour at RT, then concentrated in vacuo. The residue was purified by prep. HPLC to give Intermediate 33 (216 mg, 95% purity, 87% yield) as a yellow foam. LC-MS (Method 3): Rₜ=2.04 min; MS (ESIpos): m/z=950 [M+H]⁺.

### Example S34: Preparation of trifluoroacetic acid. N-[(benzyloxy)carbonyl]-N-methyl-L-valyl-N-[(3R,4S,5S)-3-methoxy-1-{(2S)-2-[(1R,2R)-1-methoxy-2-methyl-3-oxo-3-{[(1S,2R)-1-phenyl-1-(L-valyloxy)propan-2-yl]amino}propyl]pyrrolidin-1-yl}-5-methyl-1-oxoheptan-4-yl]-N-methyl-L-valinamide (1/1) (Intermediate 34)

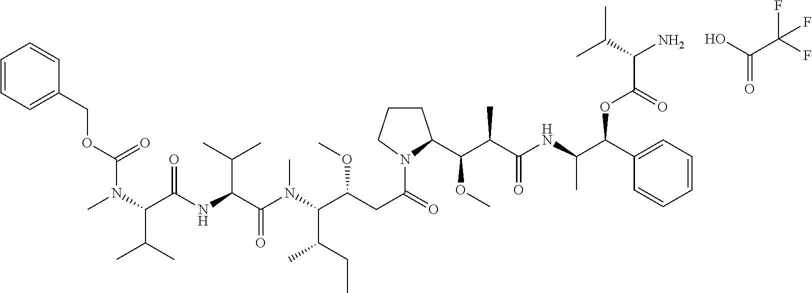 [SMILES: CC[C@H](C)[C@@H]([C@@H](CC(=O)N1CCC[C@H]1[C@H](OC)[C@@H](C)C(=O)N[C@H](C)[C@@H](OC(=O)[C@@H](N)C(C)C)c1ccccc1)OC)N(C)C(=O)[C@@H](NC(=O)[C@H](C(C)C)N(C)C(=O)OCc1ccccc1)C(C)C ; O=C(O)C(F)(F)F]

Step 1: To a solution of Monomethyl auristatin E (N-methyl-L-valyl-N-[(3R,4S,5S)-1-{(2S)-2-[(1R,2R)-3-{[(1S,2R)-1-hydroxy-1-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide, 200 mg, 279 μmol) in DMF (28 ml) were added 1-{[(benzyloxy)carbonyl]oxy}pyrrolidine-2,5-dione (83.3 mg, 334 μmol) and DIEA (150 μl, 840 μmol). The mixture was stirred at rt for 20 h and then concentrated under reduced pressure. The residue was purified by preparative HPLC and lyophilized to afford N-[(benzyloxy)carbonyl]-N-methyl-L-valyl-N-[(3R,4S,5S)-1-{(2S)-2-[(1R,2R)-3-{[(1S,2R)-1-hydroxy-1-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide (224 mg, 100% purity, 94% yield). LC-MS (Method 2): Rₜ=2.29 min; MS (ESIpos): m/z=853 [M+H]⁺.

Step 2: To a solution of N-[(benzyloxy)carbonyl]-N-methyl-L-valyl-N-[(3R,4S,5S)-1-{(2S)-2-[(1R,2R)-3-{[(1S,2R)-1-hydroxy-1-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide (222 mg, 100% purity, 260 μmol) in DCM (40 ml) were added tert-butyl (4S)-4-methyl-2,5-dioxo-1,3-oxazolidine-3-carboxylate (190 mg, 781 μmol) and DMAP (63.6 mg, 521 μmol). The mixture was refluxed for 16 h and then concentrated under reduced pressure. The residue was purified by preparative HPLC and lyophilized to afford N-[(benzyloxy)carbonyl]-N-methyl-L-valyl-N-[(3R,4S,5S)-1-{(2S)-2-[(1R,2R)-3-{[(1S,2R)-1-{[N-(tert-butoxycarbonyl)-L-valyl]oxy}-1-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide (267 mg, 100% purity, 98% yield). LC-MS (Method 3): Rₜ=6.35 min; MS (ESIpos): m/z=1052 [M+H]⁺.

Step 3: To a solution of N-[(benzyloxy)carbonyl]-N-methyl-L-valyl-N-[(3R,4S,5S)-1-{(2S)-2-[(1R,2R)-3-{[(1S,2R)-1-([N-(tert-butoxycarbonyl)-L-valyl]oxy)-1-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide (323 mg, 100% purity, 307 μmol) in DCM (50 ml), was added TFA (5.0 ml). The mixture was stirred at rt for 2 h and then concentrated under reduced pressure. The residue was dissolved in ACN/H₂O and lyophilized to afford Intermediate 34 (339 mg, 100% purity, quant.) as an amorphous residue. LC-MS (Method 2): Rₜ=1.80 min; MS (ESIpos): m/z=952 [M+H]⁺.

### Example S35: Preparation of (1S,2R)-2-({(2R,3R)-3-[(2S)-1-{(3R,4S,5S)-4-[{N-[(benzyloxy)carbonyl]-N-methyl-L-valyl-L-valyl}(methyl)amino]-3-methoxy-5-methylheptanoyl}pyrrolidin-2-yl]-3-methoxy-2-methylpropanoyl}amino)-1-phenylpropyl N-(3-{2-[2-(2-aminoethoxy)ethoxy]ethoxy}propanoyl)-L-alpha-aspartyl-L-prolyl-L-valinate.trifluoroacetic acid (1/1) (Intermediate 35)

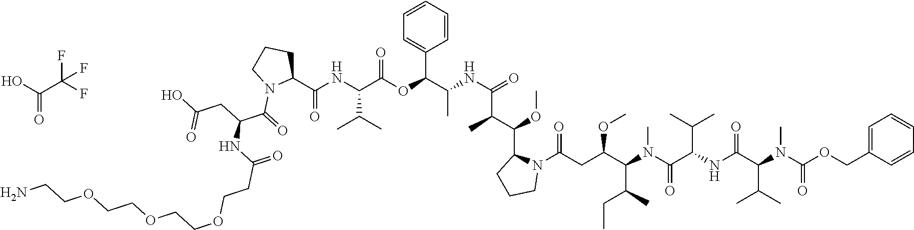 [SMILES: O=C(O)C(F)(F)F ; CC[C@H](C)[C@@H]([C@@H](CC(=O)N1CCC[C@H]1[C@H](OC)[C@@H](C)C(=O)N[C@H](C)[C@@H](OC(=O)[C@@H](NC(=O)[C@@H]1CCCN1C(=O)[C@H](CC(=O)O)NC(=O)CCOCCOCCOCCN)C(C)C)c1ccccc1)OC)N(C)C(=O)[C@@H](NC(=O)[C@H](C(C)C)N(C)C(=O)OCc1ccccc1)C(C)C]

Step 1: To a solution of trifluoroacetic acid. N-[(benzyloxy)carbonyl]-N-methyl-L-valyl-N-[(3R,4S,5S)-3-methoxy-1-((2S)-2-[(1R,2R)-1-methoxy-2-methyl-3-oxo-3-{[(1S,2R)-1-phenyl-1-(L-valyloxy)propan-2-yl]amino}propyl]pyrrolidin-1-yl)-5-methyl-1-oxoheptan-4-yl]-N-methyl-L-valinamide (1/1) (339 mg, 100% purity, 318 μmol) (Intermediate 34) in DMF (20 ml) were added (2S)-1-[(19S)-19-(2-tert-butoxy-2-oxoethyl)-2,2-dimethyl-4,17,20-trioxo-3,8,11,14-tetraoxa-5,18-diazaicosan-20-yl]pyrrolidine-2-carboxylic acid (206 mg, 350 μmol) (Intermediate 3), HATU (193 mg, 508 μmol) and DIEA (170 μl, 950 μmol). The mixture was stirred at rt for 1 h 30 and then concentrated under reduced pressure. The residue was purified by preparative HPLC and lyophilized to afford tert-butyl (19S)-19-[(2S)-2-({(2S)-1-[(1S,2R)-2-{[(2R,3R)-3-{(2S)-1-[(5S,8S,11S,12R)-11-[(2S)-butan-2-yl]-12-methoxy-4,10-dimethyl-3,6,9,14-tetraoxo-1-phenyl-5,8-di(propan-2-yl}-2-oxa-4,7,10-triazatetradecan-14-yl]pyrrolidin-2-yl)-3-methoxy-2-methylpropanoyl]amino}-1-phenylpropoxy]-3-methyl-1-oxobutan-2-yl}carbamoyl)pyrrolidine-1-carbonyl]-2,2-dimethyl-4,17-dioxo-3,8,11,14-tetraoxa-5,18-diazahenicosan-21-oate (447 mg, 100% purity, 92% yield) as an amorphous residue. LC-MS (Method 3): Rₜ=6.26 min; MS (ESIpos): m/z=1523 [M+H]⁺.

Step 2: To a solution of tert-butyl (19S)-19-[(2S)-2-({(2S)-1-[(1S,2R)-2-{[(2R,3R)-3-((2S)-1-[(5S,8S,11S,12R)-11-[(2S)-butan-2-yl]-12-methoxy-4,10-dimethyl-3,6,9,14-tetraoxo-1-phenyl-5,8-di(propan-2-yl)-2-oxa-4,7,10-triazatetradecan-14-yl]pyrrolidin-2-yl}-3-methoxy-2-methylpropanoyl]amino}-1-phenylpropoxy]-3-methyl-1-oxobutan-2-yl)carbamoyl)pyrrolidine-1-carbonyl]-2,2-dimethyl-4,17-dioxo-3,8,11,14-tetraoxa-5,18-diazahenicosan-21-oate (447 mg, 100% purity, 294 μmol) in DCM (50 ml), was added TFA (5 ml). The mixture was stirred at rt for 2 h and then concentrated under reduced pressure. The residue was dissolved in ACN/H₂O and lyophilized to afford Intermediate 35 (497 mg, 93% purity, quant.) as an amorphous residue. LC-MS (Method 3): Rₜ=4.16 min; MS (ESIpos): m/z=1367 [M+H]⁺.

### Example S36: Preparation of (1S,2R)-2-({(2R,3R)-3-[(2S)-1-{(3R,4S,5S)-4-[(N-[(benzyloxy)carbonyl]-N-methyl-L-valyl-L-valyl}(methyl)amino]-3-methoxy-5-methylheptanoyl)pyrrolidin-2-yl]-3-methoxy-2-methylpropanoyl}amino)-1-phenylpropyl N-[3-(2-{2-[2-(L-lysylamino)ethoxy]ethoxy}ethoxy)propanoyl]-L-alpha-aspartyl-L-prolyl-L-valinate trifluoroacetic acid (1/2) (Intermediate 36)

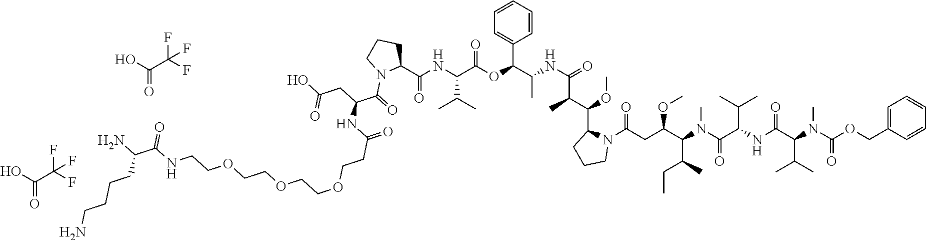 [SMILES: O=C(O)C(F)(F)F ; CC[C@H](C)[C@@H]([C@@H](CC(=O)N1CCC[C@H]1[C@H](OC)[C@@H](C)C(=O)N[C@H](C)[C@@H](OC(=O)[C@@H](NC(=O)[C@@H]1CCCN1C(=O)[C@H](CC(=O)O)NC(=O)CCOCCOCCOCCNC(=O)[C@@H](N)CCCCN)C(C)C)c1ccccc1)OC)N(C)C(=O)[C@@H](NC(=O)[C@H](C(C)C)N(C)C(=O)OCc1ccccc1)C(C)C ; O=C(O)C(F)(F)F]

Step 1:(1S,2R)-2-({(2R,3R)-3-[(2S)-1-{(3R,4S,5S)-4-[{N-[(benzyloxy)carbonyl]-N-methyl-L-valyl-L-valyl}(methyl)amino]-3-methoxy-5-methylheptanoyl}pyrrolidin-2-yl]-3-methoxy-2-methylpropanoyl}amino)-1-phenylpropyl N-(3-{2-[2-(2-aminoethoxy)ethoxy]ethoxy}propanoyl)-L-alpha-aspartyl-L-prolyl-L-valinate.trifluoroacetic acid (1/1) (245 mg, 93% purity, 153 μmol) (Intermediate 35) was dissolved in 25 mL DMF and 2,5-dioxopyrrolidin-1-yl N²,N⁶-bis(tert-butoxycarbonyl)-L-lysinate (88.3 mg, 199 μmol) as well as 80 μL N,N-diisopropylethylamine were added. After stirring for 2 h at rt the mixture was concentrated in vacuo and the residue was purified by preparative HPLC to yield (1S,2R)-2-({(2R,3R)-3-[(2S)-1-{(3R,4S,5S)-4-[{N-[(benzyloxy)carbonyl]-N-methyl-L-valyl-L-valyl}(methyl)amino]-3-methoxy-5-methylheptanoyl}pyrrolidin-2-yl]-3-methoxy-2-methylpropanoyl}amino)-1-phenylpropyl N-(3-{2-[2-(2-{[N²,N⁶-bis(tert-butoxycarbonyl)-L-lysyl]amino}ethoxy)ethoxy]ethoxy}propanoyl)-L-alpha-aspartyl-L-prolyl-L-valinate (205 mg, 100% purity, 79% yield). LC-MS (Method 3): Rₜ=5.73 min; MS (ESIpos): m/z=1695 [M+H]⁺.

Step 2: (1S,2R)-2-({(2R,3R)-3-[(2S)-1-{(3R,4S,5S)-4-[{N-[(benzyloxy)carbonyl]-N-methyl-L-valyl-L-valyl}(methyl)amino]-3-methoxy-5-methylheptanoyl}pyrrolidin-2-yl]-3-methoxy-2-methylpropanoyl}amino)-1-phenylpropyl N-(3-{2-[2-(2-{[N²,N⁶-bis(tert-butoxycarbonyl)-L-lysyl]amino}ethoxy)ethoxy]ethoxy}propanoyl)-L-alpha-aspartyl-L-prolyl-L-valinate (204 mg, 100% purity, 120 μmol) was dissolved in 20 ml DCM and 5 ml TFA was added and the reaction mixture was stirred for 1 h 30 at rt and concentrated in vacuo, The residue was dissolved in ACN/H₂O and lyophilized to give Intermediate 36 as an amorphous residue. (205 mg, 100% purity, 99% yield). LC-MS (Method 3): Rₜ=3.56 min; MS (ESIpos): m/z=1495 [M+H]⁺.

### Example S37: Preparation of (1S,2R)-2-({(2R,3R)-3-[(2S)-1-{(3R,4S,5S)-4-[{N-[(benzyloxy)carbonyl]-N-methyl-L-valyl-L-valyl}(methyl)amino]-3-methoxy-5-methylheptanoyl}pyrrolidin-2-yl]-3-methoxy-2-methylpropanoyl)amino)-1-phenylpropyl N-(3-(2-[2-(2-{[N²,N⁶-bis(3-(2-[2-(2-aminoethoxy)ethoxy]ethoxy}propanoyl)-L-lysyl]amino)ethoxy)ethoxy]ethoxy}propanoyl)-L-alpha-aspartyl-L-prolyl-L-valinate.trifluoroacetic acid (1/2) (Intermediate 37)

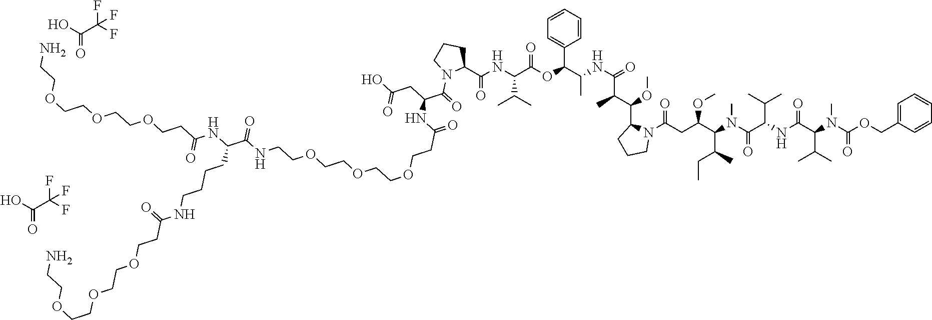 [SMILES: O=C(O)C(F)(F)F ; CC[C@H](C)[C@@H]([C@@H](CC(=O)N1CCC[C@H]1[C@H](OC)[C@@H](C)C(=O)N[C@H](C)[C@@H](OC(=O)[C@@H](NC(=O)[C@@H]1CCCN1C(=O)[C@H](CC(=O)O)NC(=O)CCOCCOCCOCCNC(=O)[C@H](CCCCNC(=O)CCOCCOCCOCCN)NC(=O)CCOCCOCCOCCN)C(C)C)c1ccccc1)OC)N(C)C(=O)[C@@H](NC(=O)[C@H](C(C)C)N(C)C(=O)OCc1ccccc1)C(C)C ; O=C(O)C(F)(F)F]

Step 1: (1S,2R)-2-({(2R,3R)-3-[(2S)-1-{(3R,4S,5S)-4-[{N-[(benzyloxy)carbonyl]-N-methyl-L-valyl-L-valyl}(methyl)amino]-3-methoxy-5-methylheptanoyl}pyrrolidin-2-yl]-3-methoxy-2-methylpropanoyl}amino)-1-phenylpropyl N-[3-(2-{2-[2-(L-lysylamino)ethoxy]ethoxy}ethoxy)propanoyl]-L-alpha-aspartyl-L-prolyl-L-valinate.trifluoroacetic acid (1/2) (204 mg, 119 μmol) (Intermediate 36) was dissolved in 25 mL DM4F and tert-butyl {2-[2-(2-{3-[(2,5-dioxopyrrolidin-1-yl)oxy]-3-oxopropoxy}ethoxy)ethoxy]ethyl}carbaminate (119 mg, 284 μmol μmol) as well as 170 μL N,N-diisopropylethylamine were added. After stirring for 2 h at rt the mixture was concentrated in vacuo and the residue was purified by prep. HPLC to yield (1S,2R)-2-({(2R,3R)-3-[(2S)-1-{(3R,4S,5S)-4-[{N-[(benzyloxy)carbonyl]-N-methyl-L-valyl-L-valyl}(methyl)amino]-3-methoxy-5-methylheptanoyl}pyrrolidin-2-yl]-3-methoxy-2-methylpropanoyl}amino)-1-phenylpropyl N-(3-{2-[2-(2-{[N²,N⁶-bis(2,2-dimethyl-4,17-dioxo-3,8,11,14-tetraoxa-5-azaheptadecan-17-yl)-L-lysyl]amino}ethoxy)ethoxy]ethoxy}propanoyl)-L-alpha-aspartyl-L-prolyl-L-valinate (207 mg, 100% purity, 83% yield) as an amorphous residue. LC-MS (Method 3): Rₜ=5.48 min; MS (ESIpos): m/z=2101 [M+H]⁺.

Step 2: (1S,2R)-2-({(2R,3R)-3-[(2S)-1-{(3R,4S,5S)-4-[{N-[(benzyloxy)carbonyl]-N-methyl-L-valyl-L-valyl}(methyl)amino]-3-methoxy-5-methylheptanoyl}pyrrolidin-2-yl]-3-methoxy-2-methylpropanoyl}amino)-1-phenylpropyl N-(3-{2-[2-(2-{[N²,N⁶-bis(2,2-dimethyl-4,17-dioxo-3,8,11,14-tetraoxa-5-azaheptadecan-17-yl)-L-lysyl]amino}ethoxy)ethoxy]ethoxy}propanoyl)-L-alpha-aspartyl-L-prolyl-L-valinate (206 mg, 100% purity, 98.2 μmol) was dissolved in 20 ml DCM and 5 ml TFA was added and the reaction mixture was stirred for 1 h at rt and concentrated in vacuo. The residue was dissolved in ACN/H₂O and lyophilized to give Intermediate 37 (206 mg, 100% purity, 98% yield) as an amorphous residue. LC-MS (Method 3): Rₜ=3.54 min; MS (ESIpos): m/z=1901 [M+H].

### Example S38: Preparation of N-{3-[{(1R)-1-[1-benzyl-4-(2,5-difluorophenyl)-1H-pyrrol-2-yl]-2,2-dimethylpropyl}(hydroxyacetyl)amino]propyl}-L-alaninamide (1/1) (Intermediate 38)

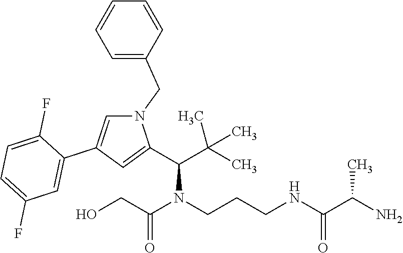 [SMILES: C[C@H](N)C(=O)NCCCN(C(=O)CO)[C@@H](c1cc(-c2cc(F)ccc2F)cn1Cc1ccccc1)C(C)(C)C]

Step 1: To a solution of trifluoroacetic acid. N-(3-aminopropyl)-N-{(1R)-1-[1-benzyl-4-(2,5-difluorophenyl)-1H-pyrrol-2-yl]-2,2-dimethylpropyl}-2-hydroxyacetamide (1/1) (100 mg, 171 μmol, as described in WO2015096982) in DMF (25 ml) were added 2,5-dioxopyrrolidin-1-yl N-[(benzyloxy)carbonyl]-L-alaninate (110 mg, 343 μmol) and DIEA (120 μl, 690 μmol). The mixture was stirred at rt for 1 h and concentrated under reduced pressure. The residue was purified over preparative HPLC to afford N-{3-[{(1R)-1-[1-benzyl-4-(2,5-difluorophenyl)-1H-pyrrol-2-yl]-2,2-dimethylpropyl}(hydroxyacetyl)amino]propyl}-L-alaninamide (101 mg, 98% purity, 86% yield) as a colorless foam. LC-MS (Method 3): Rₜ=5.43 min; MS (ESIpos): m/z=675 [M+H]⁺.

Step 2: To a solution of benzyl [(2S)-1-({3-[{(1R)-1-[1-benzyl-4-(2,5-difluorophenyl)-1H-pyrrol-2-yl]-2,2-dimethylpropyl}(hydroxyacetyl)amino]propyl}amino)-1-oxopropan-2-yl]carbamate (101 mg, 150 μmol) in DCM (12.5 ml) and methanol (12.5 ml) was added Pd/C (20 mg). The reaction was hydrogenated at rt for 2 h and filtered. The mother liquor was concentrated in vacuo and lyophilized to give Intermediate 38 (80 mg) as a colorless foam which was used in the next step without further purification.

### Example S39: Preparation of (2S)-1-[(2S)-4-amino-2-[3-[2-[2-[2-(tert-butoxycarbonylamino)ethoxy]ethoxy]ethoxy]propanoylamino]-4-oxo-butanoyl]pyrrolidine-2-carboxylic acid (Intermediate 39)

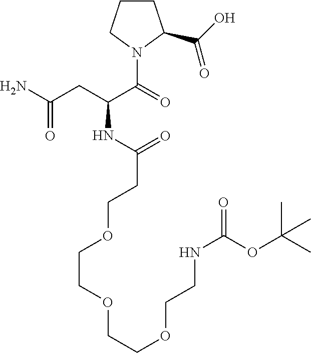 [SMILES: CC(C)(C)OC(=O)NCCOCCOCCOCCC(=O)N[C@@H](CC(N)=O)C(=O)N1CCC[C@H]1C(=O)O]

Intermediate 39 was synthesized using classical peptide synthesis methods starting with the coupling of (2S)-4-amino-2-(tert-butoxycarbonylamino)-4-oxo-butanoic acid with benzyl (2S)-pyrrolidine-2-carboxylate.hydrochloride in DMF in the presence of N,N-Diisopropylethylamine and subsequent removal of the Boc-protecting group with TFA in DCM. This partially protected dipeptide was acylated with tert-butyl {2-[2-(2-{3-[(2,5-dioxopyrrolidin-1-yl)oxy]-3-oxopropoxy}ethoxy)ethoxy]ethyl}carbamate (Intermediate 10) in DMF in the presence of N, N-Diisopropylethylamine. In the final step the benzylester was removed by hydrogenolysis over 10% Pd/charcoal. LC-MS (Method 2): R: =1.00 min; MS (ESIpos): m/z=533 [M+H]⁺.

### Example S40: Preparation of trifluoroacetic acid. N²-(3-{2-[2-(2-aminoethoxy)ethoxy]ethoxy}propanoyl)-L-asparaginyl-L-prolyl-N-{3-[{(1R)-1-[1-benzyl-4-(2,5-difluorophenyl)-1H-pyrrol-2-yl]-2,2-dimethylpropyl}(hydroxyacetyl)amino]propyl}-L-alaninamide (1/1) (Intermediate 40)

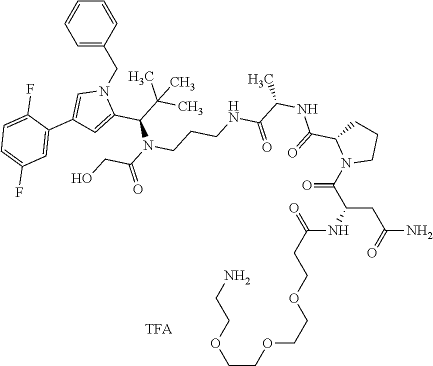 [SMILES: C[C@H](NC(=O)[C@@H]1CCCN1C(=O)[C@H](CC(N)=O)NC(=O)CCOCCOCCOCCN)C(=O)NCCCN(C(=O)CO)[C@@H](c1cc(-c2cc(F)ccc2F)cn1Cc1ccccc1)C(C)(C)C]

Step 1: To a solution of N-{3-[{(1R)-1-[1-benzyl-4-(2,5-difluorophenyl)-1H-pyrrol-2-yl]-2,2-dimethylpropyl}(hydroxyacetyl)amino]propyl}-L-alaninamide (40.0 mg, 74.0 μmol) (Intermediate 38) in DMF (10 ml) were added N²-(2,2-dimethyl-4,17-dioxo-3,8,11,14-tetraoxa-5-azaheptadecan-17-yl)-L-asparaginyl-L-proline (47.3 mg, 88.8 μmol) (Intermediate 39), HATU (36.6 mg, 96.2 μmol) and DIEA (39 μl, 220 μmol). The mixture was stirred at rt for 2h and then concentrated under reduced pressure. The residue was purified by preparative HPLC to afford N²-(2,2-dimethyl-4,17-dioxo-3,8,11,14-tetraoxa-5-azaheptadecan-17-yl)-L-asparaginyl-L-prolyl-N-{3-[{(1R)-1-[1-benzyl-4-(2,5-difluorophenyl)-1H-pyrrol-2-yl]-2,2-dimethylpropyl}(hydroxyacetyl)amino]propyl}-L-alaninamide (62 mg, 96% purity, 77% yield) as a light yellow foam. LC-MS (Method 3): Rₜ=4.83 min; MS (ESIpos): m/z=1056 [M+H]⁺

Step 2: To a solution of N²-(2,2-dimethyl-4,17-dioxo-3,8,11,14-tetraoxa-5-azaheptadecan-17-yl)-L-asparaginyl-L-prolyl-N-{3-[{(1R)-1-[1-benzyl-4-(2,5-difluorophenyl)-1H-pyrrol-2-yl]-2,2-dimethylpropyl}(hydroxyacetyl)amino]propyl}-L-alaninamide (62.0 mg, 58.8 μmol) in trifluoroethanol (5 ml), was added zinc chloride (64.1 mg, 470 μmol). The mixture was stirred at 50° C. for 5 h. EDTA (137 mg, 470 μmol) and water 0.1% TFA (4 ml) were then added and the resulting mixture was purified over preparative HPLC to afford Intermediate 40 (55 mg, 100% purity, 88% yield) as a colorless foam. LC-MS (Method 3): Rₜ=3.46 min; MS (ESIpos): m/z=956 [M+H]⁺.

### Example S41: Preparation of N-{3-[{(1R)-1-[1-benzyl-4-(2,5-difluorophenyl)-1H-pyrrol-2-yl]-2,2-dimethylpropyl}(hydroxyacetyl)amino]propyl}-L-valinamide (Intermediate 41)

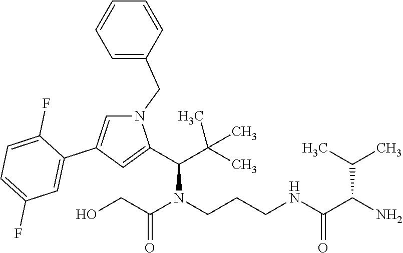 [SMILES: CC(C)[C@H](N)C(=O)NCCCN(C(=O)CO)[C@@H](c1cc(-c2cc(F)ccc2F)cn1Cc1ccccc1)C(C)(C)C]

Step 1: To a solution of trifluoroacetic acid. N-(3-aminopropyl)-N-{(1R)-1-[1-benzyl-4-(2,5-difluorophenyl)-1H-pyrrol-2-yl]-2,2-dimethylpropyl}-2-hydroxyacetamide (1/1) (250 mg, 428 μmol, as described in WO2015096982) in DMF (20 ml) were added 2,5-dioxopyrrolidin-1-yl N-[(benzyloxy)carbonyl]-L-valinate (298 mg, 857 μmol) and DIEA (300 μl, 1.7 mmol). The mixture was stirred at rt for 1 h and then diluted with water and ethyl acetate. The layers were separated and the organic phase was then concentrated under reduced pressure. The residue was purified over preparative HPLC and lyophilized to N-{3-[{(1R)-1-[1-benzyl-4-(2,5-difluorophenyl)-1H-pyrrol-2-yl]-2,2-dimethylpropyl}(hydroxyacetyl)amino]propyl}-N²-[(benzyloxy)carbonyl]-L-valinamide (252 mg, 98% purity, 82% yield) as an amorphous residue. LC-MS (Method 3): Rₜ=5.72 min; MS (ESIpos): m/z=703 [M+H]⁺

Step 2: To a solution of N-{3-[{(1R)-1-[1-benzyl-4-(2,5-difluorophenyl)-1H-pyrrol-2-yl]-2,2-dimethylpropyl}(hydroxyacetyl)amino]propyl}-N²-[(benzyloxy)carbonyl]-L-valinamide (250 mg, 98% purity, 349 μmol) in DCM (15 ml) and methanol (15 ml) was added Pd/C (45 mg). The reaction was hydrogenated at rt for 2h 30 and filtered over celite. The mother liquor was concentrated in vacuo to give Intermediate 41 (117 mg, 100% purity, 59% yield) as a colorless resin which was used in the next step without further purification.

### Example S42: Preparation of trifluoroacetic acid. N²-(3-{2-[2-(2-aminoethoxy)ethoxy]ethoxy}propanoyl)-L-asparaginyl-L-prolyl-N-{3-[{(1R)-1-[1-benzyl-4-(2,5-difluorophenyl)-1H-pyrrol-2-yl]-2,2-dimethylpropyl}(hydroxyacetyl)amino]propyl}-L-valinamide (1/1) (Intermediate 42)

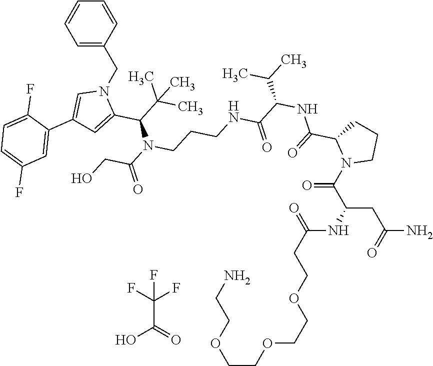 [SMILES: CC(C)[C@H](NC(=O)[C@@H]1CCCN1C(=O)[C@H](CC(N)=O)NC(=O)CCOCCOCCOCCN)C(=O)NCCCN(C(=O)CO)[C@@H](c1cc(-c2cc(F)ccc2F)cn1Cc1ccccc1)C(C)(C)C ; O=C(O)C(F)(F)F]

Step 1: To a solution of N-{3-[{(1R)-1-[1-benzyl-4-(2,5-difluorophenyl)-1H-pyrrol-2-yl]-2,2-dimethylpropyl}(hydroxyacetyl)amino]propyl}-L-valinamide (115 mg, 202 μmol) (Intermediate 41) in DMF (20 ml) were added N²-(2,2-dimethyl-4,17-dioxo-3,8,11,14-tetraoxa-5-azaheptadecan-17-yl)-L-asparaginyl-L-proline (140 mg, 92% purity, 243 μmol) (Intermediate 39), HATU (100 mg, 263 μmol) and DIEA (110 μl, 610 μmol). The mixture was stirred at rt for 2 h and then concentrated under reduced pressure. The residue was purified by preparative HPLC and lyophilized to afford N²-(2,2-dimethyl-4,17-dioxo-3,8,11,14-tetraoxa-5-azaheptadecan-17-yl)-L-asparaginyl-L-prolyl-N-{3-[{(1R)-1-[1-benzyl-4-(2,5-difluorophenyl)-1H-pyrrol-2-yl]-2,2-dimethylpropyl}(hydroxyacetyl)amino]propyl}-L-valinamide (153 mg, 76% purity, 53% yield) as an amorphous residue. LC-MS (Method 3): Rₜ=5.01 min; MS (ESIpos): m/z=1084 [M+H]⁺.

Step 2: To a solution of N²-(2,2-dimethyl-4,17-dioxo-3,8,11,14-tetraoxa-5-azaheptadecan-17-yl)-L-asparaginyl-L-prolyl-N-{3-[{(1R)-1-[1-benzyl-4-(2,5-difluorophenyl)-1H-pyrrol-2-yl]-2,2-dimethylpropyl}(hydroxyacetyl)amino]propyl}-L-valinamide (280 mg, 96% purity, 247 μmol) in Trifluoroethanol (20 ml), was added zinc chloride (269 mg, 1.98 mmol). The mixture was stirred at 50° C. for 5 h. EDTA (577 mg, 1.98 mmol) and water 0.1% TFA (6 ml) were then added and the resulting mixture was purified over preparative HPLC and lyophilized to afford Intermediate 42 (192 mg, 100% purity, 71% yield) as an amorphous residue. LC-MS (Method 3): Rₜ=3.62 min; MS (ESIpos): m/z=984 [M+H]⁺.

### Example S43: Preparation of (4S)-4,11-Diethyl-3,14-dioxo-3,4,12,14-tetrahydro-1H-pyrano[3′,4′:6,7]indolizino[1,2-b]quinolin-4-yl N-[3-(2-{2-[2-(L-lysylamino)ethoxy]ethoxy}ethoxy)propanoyl]-L-alpha-aspartyl-L-prolyl-L-valinate⋅trifluoroacetic acid (1:1) (Intermediate 43)

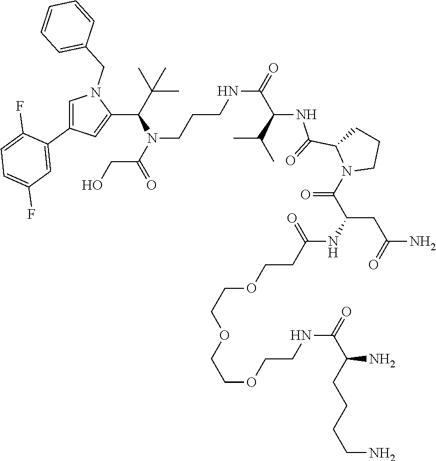 [SMILES: CC(C)[C@H](NC(=O)[C@@H]1CCCN1C(=O)[C@H](CC(N)=O)NC(=O)CCOCCOCCOCCNC(=O)[C@@H](N)CCCCN)C(=O)NCCCN(C(=O)CO)[C@@H](c1cc(-c2cc(F)ccc2F)cn1Cc1ccccc1)C(C)(C)C]

Step 1: Trifluoroacetic acid. N²-(3-{2-[2-(2-aminoethoxy)ethoxy]ethoxy}propanoyl)-L-asparaginyl-L-prolyl-N-{3-[{(1R)-1-[1-benzyl-4-(2,5-difluorophenyl)-1H-pyrrol-2-yl]-2,2-dimethylpropyl}(hydroxyacetyl)amino]propyl}-L-valinamide (1/1) (290 mg, 264 μmol) (Intermediate 42) was dissolved in 30 mL DMF and 2,5-dioxopyrrolidin-1-yl N²,N⁶-bis[(benzyloxy)carbonyl]-L-lysinate (185 mg, 95% purity, 344 μmol) as well as 92 μL N,N-diisopropylethylamine were added. After stirring for 1 h at rt, the mixture was concentrated in vacuo and the residual was purified by preparative HPLC to yield N²-[3-(2-{2-[2-({N²,N⁶-bis[(benzyloxy)carbonyl]-L-lysyl}amino)ethoxy]ethoxy}ethoxy)propanoyl]-L-asparaginyl-L-prolyl-N-{3-[{(1R)-1-[1-benzyl-4-(2,5-difluorophenyl)-1H-pyrrol-2-yl]-2,2-dimethylpropyl}(hydroxyacetyl)amino]propyl}-L-valinamide (295 mg, 98% purity, 79% yield) as an amorphous residue. LC-MS (Method 2): Rₜ=2.27 min; MS (ESIpos): m/z=1379 [M+H]⁺

Step 2: To a solution of N²-[3-(2-{2-[2-({N²,N⁶-bis[(benzyloxy)carbonyl]-L-lysyl}amino)ethoxy]ethoxy}ethoxy)propanoyl]-L-asparaginyl-L-prolyl-N-{3-[{(1R)-1-[1-benzyl-4-(2,5-difluorophenyl)-1H-pyrrol-2-yl]-2,2-dimethylpropyl}(hydroxyacetyl)amino]propyl}-L-valinamide (295 mg, 98% purity, 209 μmol) in DCM (15 ml) and methanol (15 ml) was added Pd/C (50 mg). The reaction was hydrogenated at rt for 2 h and filtered over celite. The mother liquor was concentrated in vacuo and lyophilized to afford Intermediate 43 (205 mg, 92% purity, 81% yield) as a white amorphous residue. LC-MS (Method 3): Rₜ=3.32 min; MS (ESIpos): m/z=1110 [M+H]⁺.

### Example S44: Preparation of trifluoroacetic acid. N²-(3-{2-[2-(2-{[N²,N⁶-bis(3-(2-[2-(2-aminoethoxy)ethoxy]ethoxy}propanoyl)-L-lysyl]amino)ethoxy)ethoxy]ethoxy}propanoyl)-L-asparaginyl-L-prolyl-N-{3-[{(1R)-1-[1-benzyl-4-(2,5-difluorophenyl)-1H-pyrrol-2-yl]-2,2-dimethylpropyl}(hydroxyacetyl)amino]propyl}-L-valinamide (2/1) (Intermediate 44)

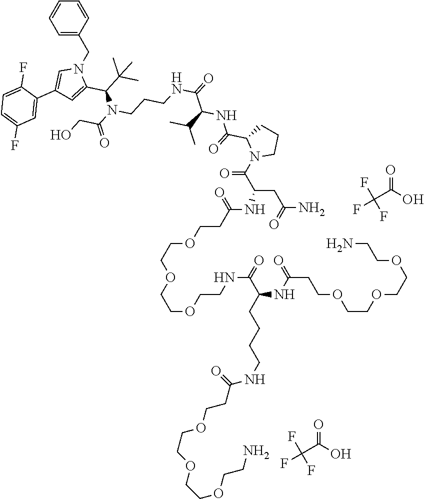 [SMILES: CC(C)[C@H](NC(=O)[C@@H]1CCCN1C(=O)[C@H](CC(N)=O)NC(=O)CCOCCOCCOCCNC(=O)[C@H](CCCCNC(=O)CCOCCOCCOCCN)NC(=O)CCOCCOCCOCCN)C(=O)NCCCN(C(=O)CO)[C@@H](c1cc(-c2cc(F)ccc2F)cn1Cc1ccccc1)C(C)(C)C ; O=C(O)C(F)(F)F ; O=C(O)C(F)(F)F]

Step 1: (N²-[3-(2-{2-[2-(L-lysylamino)ethoxy]ethoxy}ethoxy)propanoyl]-L-asparaginyl-L-prolyl-N-{3-[{(1R)-1-[1-benzyl-4-(2,5-difluorophenyl)-1H-pyrrol-2-yl]-2,2-dimethylpropyl}(hydroxyacetyl)amino]propyl}-L-valinamide (205 mg, 92% purity, 170 μmol) (Intermediate 43) was dissolved in 30 mL DMF and 2.2 eq tert-butyl {2-[2-(2-{3-[(2,5-dioxopyrrolidin-1-yl)oxy]-3-oxopropoxy}ethoxy)ethoxy]ethyl}carbamate (170 mg, 407 μmol) as well as 180 μL N,N-diisopropylethylamine were added. After stirring for 4 h at rt the mixture was concentrated in vacuo and the residual was purified by preparative HPLC and lyophilized to yield N²-(3-{2-[2-(2-{[N²,N⁶-bis(2,2-dimethyl-4,17-dioxo-3,8,11,14-tetraoxa-5-azaheptadecan-17-yl)-L-lysyl]amino}ethoxy)ethoxy]ethoxy}propanoyl)-L-asparaginyl-L-prolyl-N-{3-[{(1R)-1-[1-benzyl-4-(2,5-difluorophenyl)-1H-pyrrol-2-yl]-2,2-dimethylpropyl}(hydroxyacetyl)amino]propyl}-L-valinamide (250 mg, 93% purity, 80% yield) as an amorphous residue. LC-MS (Method 3): Rₜ=5.20 min; MS (ESIpos): m/z=1716 [M+H]⁺

Step 2: To a solution of N²-(3-{2-[2-(2-{[N²,N⁶-bis(2,2-dimethyl-4,17-dioxo-3,8,11,14-tetraoxa-5-azaheptadecan-17-yl)-L-lysyl]amino}ethoxy)ethoxy]ethoxy}propanoyl)-L-asparaginyl-L-prolyl-N-{3-[{(1R)-1-[1-benzyl-4-(2,5-difluorophenyl)-1H-pyrrol-2-yl]-2,2-dimethylpropyl}(hydroxyacetyl)amino]propyl}-L-valinamide (250 mg, 93% purity, 135 μmol) in Trifluoroethanol (12 ml), was added zinc chloride (148 mg, 1.08 mmol). The mixture was stirred at 50° C. for 2h 30. The resulting mixture was diluted in acetonitrile/water and EDTA (317 mg, 1.08 mmol) was then added. The resulting mixture was filtered, purified over preparative HPLC and lyophilized to afford Intermediate 44 (187 mg, 100% purity, 79% yield) as an amorphous residue. LC-MS (Method 3): Rₜ=3.35 min; MS (ESIpos): m/z=1516 [M+H]⁺

### Example S45: Preparation of trifluoroacetic acid. benzyl L-prolyl-L-valinate (2/1) (Intermediate 45)

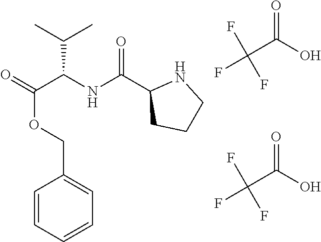

Intermediate 45 was synthesized using classical peptide synthesis methods starting with the coupling of 1-(tert-butoxycarbonyl)-L-proline with 4-methylbenzene-1-sulfonic acid. benzyl L-valinate (1/1) in DMF in the presence of HATU and N,N-Diisopropylethylamine and subsequent removal of the Boc-protecting group with TFA in DCM. LC-MS (Method 2): R.=0.94 min; MS (ESIpos): m/z=305 [M+H]⁺

### Example S46: Preparation of N²-(2,2-dimethyl-4,17-dioxo-3,8,11,14-tetraoxa-5-azaheptadecan-17-yl)-L-asparaginyl-L-prolyl-L-valine (Intermediate 46)

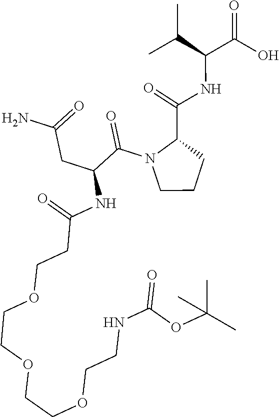 [SMILES: CC(C)[C@H](NC(=O)[C@@H]1CCCN1C(=O)[C@H](CC(N)=O)NC(=O)CCOCCOCCOCCNC(=O)OC(C)(C)C)C(=O)O]

Intermediate 46 was synthesized using classical peptide synthesis methods starting with the coupling of Intermediate 45 with 2,5-dioxopyrrolidin-1-yl N²-(tert-butoxycarbonyl)-L-asparaginate in DMF in the presence of N,N-Diisopropylethylamine and subsequent removal of the Boc-protecting group with TFA in DCM. This partially protected tripeptide was acylated with tert-butyl {2-[2-(2-{3-[(2,5-dioxopyrrolidin-1-yl)oxy]-3-oxopropoxy}ethoxy)ethoxy]ethyl}carbamate (Intermediate 10) in DMF in the presence of N, N-Diisopropylethylamine. In the final step the benzylester was removed by hydrogenolysis over 10% Pd/charcoal. LC-MS (Method 2): Rₜ=1.10 min; MS (ESIpos): m/z=632 [M+H]⁺

### Example S47: Preparation of N²-(3-{2-[2-(2-aminoethoxy)ethoxy]ethoxy}propanoyl)-L-asparaginyl-L-prolyl-N-[(2S)-4-[{(1R)-1-[1-benzyl-4-(2,5-difluorophenyl)-1H-pyrrol-2-yl]-2,2-dimethylpropyl}(hydroxyacetyl)amino]-1-(methylamino)-1-oxobutan-2-yl]-L-valinamide (Intermediate 47)

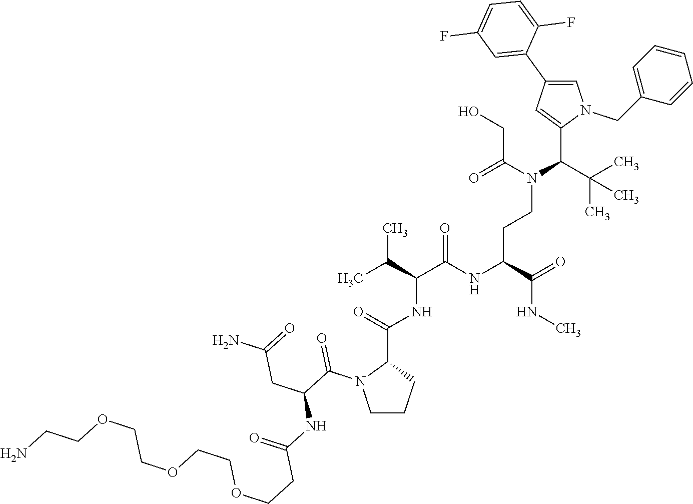 [SMILES: CNC(=O)[C@H](CCN(C(=O)CO)[C@@H](c1cc(-c2cc(F)ccc2F)cn1Cc1ccccc1)C(C)(C)C)NC(=O)[C@@H](NC(=O)[C@@H]1CCCN1C(=O)[C@H](CC(N)=O)NC(=O)CCOCCOCCOCCN)C(C)C]

Step 1: To a solution of (2S)-2-amino-4-[{(1R)-1-[1-benzyl-4-(2,5-difluorophenyl)-1H-pyrrol-2-yl]-2,2-dimethylpropyl}(hydroxyacetyl)amino]-N-methylbutanamide (63.0 mg, 120 μmol, CAS Nr: 1800460-13-2, as described in WO2015096982) in DMF (5 ml) were added N²-(2,2-dimethyl-4,17-dioxo-3,8,11,14-tetraoxa-5-azaheptadecan-17-yl)-L-asparaginyl-L-prolyl-L-valine (90.7 mg, 144 μmol) (Intermediate 46), HATU (59.1 mg, 156 μmol) and DIEA (63 μl, 360 μmol). The mixture was stirred at rt for 1 h and then concentrated under reduced pressure. The residue was purified by preparative HPLC and lyophilized to afford N²-(2,2-dimethyl-4,17-dioxo-3,8,11,14-tetraoxa-5-azaheptadecan-17-yl)-L-asparaginyl-L-prolyl-N-[(2S)-4-[{(1R)-1-[1-benzyl-4-(2,5-difluorophenyl)-1H-pyrrol-2-yl]-2,2-dimethylpropyl}(hydroxyacetyl)amino]-1-(methylamino)-1-oxobutan-2-yl]-L-valinamide (92 mg, 98% purity, 66% yield) as an amorphous residue. LC-MS (Method 3): Rₜ=4.93 min; MS (ESIpos): m/z=1141 [M+H]⁺

Step 2: To a solution of N²-(2,2-dimethyl-4,17-dioxo-3,8,11,14-tetraoxa-5-azaheptadecan-17-yl)-L-asparaginyl-L-prolyl-N-[(2S)-4-[{(1R)-1-[1-benzyl-4-(2,5-difluorophenyl)-1H-pyrrol-2-yl]-2,2-dimethylpropyl}(hydroxyacetyl)amino]-1-(methylamino)-1-oxobutan-2-yl]-L-valinamide (92.0 mg, 98% purity, 79.2 μmol) in Trifluoroethanol (4 ml), was added zinc chloride (86.3 mg, 633 μmol). The mixture was stirred at 50° C. for 5 h. The mixture was freezed at −20° C. for 4 days and then stirred again at 50° C. for 3 h. EDTA (185 mg, 633 μmol) and water 0.1% TFA (3 ml) were then added and the resulting mixture was purified over preparative HPLC and lyophilized to afford Intermediate 47 (84 mg, 96% purity, 97% yield) as an amorphous residue. LC-MS (Method 3): Rₜ=3.44 min; MS (ESIpos): m/z=1041 [M+H]⁺.

### Example S48: Preparation of trifluoroacetic acid. N-[(3RS)-3-amino-4,4-difluorobutyl]-N-{(1R)-1-[1-benzyl-4-(2,5-difluorophenyl)-1H-pyrrol-2-yl]-2,2-dimethylpropyl}-2-hydroxyacetamide (1/1) (Intermediate 48)

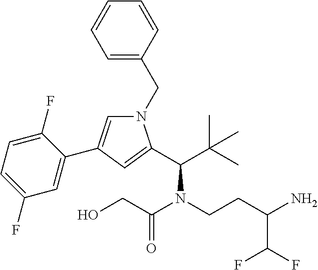 [SMILES: CC(C)(C)[C@H](c1cc(-c2cc(F)ccc2F)cn1Cc1ccccc1)N(CCC(N)C(F)F)C(=O)CO]

Step 1: To a solution of(1R)-1-[1-benzyl-4-(2,5-difluorophenyl)-1H-pyrrol-2-yl]-2,2-dimethylpropan-1-amine (159 mg, 448 μmol, as described in WO2015096982, CAS Nr: 1800455-85-9) in DCM (20 ml) were added sodium triacetoxyborohydride (133 mg, 627 μmol) and acetic acid (38 μl). The mixture was stirred at rt for 5 min and a solution of tert-butyl [(2RS)-1,1-difluoro-4-oxobutan-2-yl]carbamate (150 mg, 672 μmol) in DCM (5 ml) was then added. The mixture was stirred at rt for 3 h and concentrated under reduced pressure. The residue was diluted with ethyl acetate, washed twice with a saturated sodium carbonate solution, and once with brine. dried over magnesium sulphate, filtered and evaporated under reduced pressure to afford tert-butyl [(2RS)-4-({(1R)-1-[1-benzyl-4-(2,5-difluorophenyl)-1H-pyrrol-2-yl]-2,2-dimethylpropyl}amino)-1,1-difluorobutan-2-yl]carbamate (250 mg, 87% purity, 86% yield). LC-MS (Method 2): Rₜ=1.74 min; MS (ESIpos): m/z=562 [M+H])

Step 2: To a solution of tert-butyl [(2RS)-4-({(1R)-1-[1-benzyl-4-(2,5-difluorophenyl)-1H-pyrrol-2-yl]-2,2-dimethylpropyl}amino)-1,1-difluorobutan-2-yl]carbamate (250 mg, 87% purity, 385 μmol) in DCM (50 ml), was added, under argon, triethylamine (270 pd, 1.9 mmol). 2-Chloro-2-oxoethyl acetate (140 μl, 1.3 mmol) was the added at 0° C. and the mixture was stirred at rt for 6 h and concentrated under reduced pressure. The residue was diluted with ethyl acetate, washed with a saturated citric acid solution, water, twice with a saturated sodium hydrogen carbonate solution and once with brine. The organic phase was dried over magnesium sulphate, filtered and evaporated under reduced pressure to afford 2-({(1R)-1-[1-benzyl-4-(2,5-difluorophenyl)-1H-pyrrol-2-yl]-2,2-dimethylpropyl}{(3RS)-3-[(tert-butoxycarbonyl)amino]-4,4-difluorobutyl}amino)-2-oxoethyl acetate (326 mg, 77% purity, 99% yield) as a resinous residue. LC-MS (Method 2): Rₜ=2.59 min; MS (ESIpos): m/z=662 [M+H]⁺

Step 3: To a solution of 2-({(1R)-1-[1-benzyl-4-(2,5-difluorophenyl)-1H-pyrrol-2-yl]-2,2-dimethylpropyl}{(3RS)-3-[(tert-butoxycarbonyl)amino]-4,4-difluorobutyl}amino)-2-oxoethyl acetate (326 mg, 77% purity, 379 μmol) in THF (30 ml), were added water (15 ml) and LiOH aqueous solution (1.9 ml, 2.0 M, 3.8 mmol). The mixture was stirred at rt for 3 h and then neutralized with TFA. Ethyl acetate was then added. The organic phase was washed twice with water and brine, dried over magnesium sulphate and evaporated under reduced pressure. The residue was purified over preparative HPLC to afford tert-butyl {(2RS)-4-[{(1R)-1-[1-benzyl-4-(2,5-difluorophenyl)-1H-pyrrol-2-yl]-2,2-dimethylpropyl}(hydroxyacetyl)amino]-1,1-difluorobutan-2-yl}carbamate (180 mg, 92% purity, 70% yield) as an amorphous residue. LC-MS (Method 2): Rₜ=2.58 min; MS (ESIpos): m/z=619 [M+H]⁺.

Step 4: To a solution of tert-butyl {(2RS)-4-[{(1R)-1-[1-benzyl-4-(2,5-difluorophenyl)-1H-pyrrol-2-yl]-2,2-dimethylpropyl}(hydroxyacetyl)amino]-1,1-difluorobutan-2-yl}carbamate (180 mg, 92% purity, 267 μmol) in Trifluoroethanol (15 ml), was added zinc chloride (219 mg, 1.60 mmol). The mixture was stirred at 50° C. for 1 h 30. EDTA (469 mg, 1.60 mmol) was added and the resulting mixture was diluted with water and acetonitrile, filtered, purified over preparative HPLC and lyophilized to afford Intermediate 48 (76 mg, 100% purity, 45% yield) as an amorphous residue. LC-MS (Method 2): Rₜ=1.66 min; MS (ESIpos): m/z=520 [M+H]⁺.

### Example S51: Preparation of benzyl N-{5-[(2,5-dioxopyrrolidin-1-yl)oxy]-5-oxopentanoyl}-N-methylglycyl-N-methylglycyl-N-methylglycyl-N-methylglycyl-N-methylglycyl-N-methylglycyl-N-methylglycyl-N-methylglycinate (Intermediate 51)

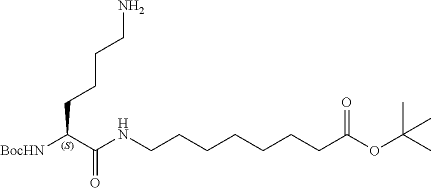

Step 1: To a solution of N⁶-[(benzyloxy)carbonyl]-N²-(tert-butoxycarbonyl)-L-lysine (600 mg, 1.58 mmol) in DMF (15 ml), were added tert-butyl 8-aminooctanoate (374 mg, 1.73 mmol), HATU (780 mg, 2.05 mmol) and N,N-diisopropylethylamine (410 μl, 2.4 mmol). The mixture was stirred at rt for 1 h. Ethyl acetate was then added and the organic layer was washed with water and brine. The organic phase was then dried over sodium sulphate and concentrated under reduced pressure. The residue was purified by preparative HPLC and lyophilized to afford tert-butyl 8-({N6-[(benzyloxy)carbonyl]-N2-(tert-butoxycarbonyl)-L-lysyl}amino)octanoate (940 mg, 89% purity, 91% yield) as an amorphous residue. LC-MS (Method 2): Rₜ=2.33 min; MS (ESIpos): m/z=578 [M+H]⁺.

Step 2: Tert-butyl 8-({N6-[(benzyloxy)carbonyl]-N2-(tert-butoxycarbonyl)-L-lysyl}amino)octanoate (938 mg, 1.62 mmol) was dissolved in methanol (25 ml) and dichloromethane (25 ml). Pd/C 10% (125 mg) was added and the reaction was hydrogenated at RT for 5 h and filtered over celite. The mother liquor was concentrated under reduced pressure to afford Intermediate 51 (674 mg, 91% purity, 85% yield) as a colorless resin. LC-MS (Method 4): Rₜ=1.41 min; MS (ESIpos): m/z=444 [M+H]⁺.

### Example S52: Preparation of(4S)-4,11-diethyl-3,14-dioxo-3,4,12,14-tetrahydro-1H-pyrano[3′,4′:6,7]indolizino[1,2-b]quinolin-4-yl N-[(24S)-24-amino-33-carboxy-14,18,25-trioxo-4,7,10-trioxa-13,19,26-triazatritriacontanan-1-oyl]-L-alpha-aspartyl-L-prolyl-L-valinate-trifluoroacetic acid (1/2) (Intermediate 52)

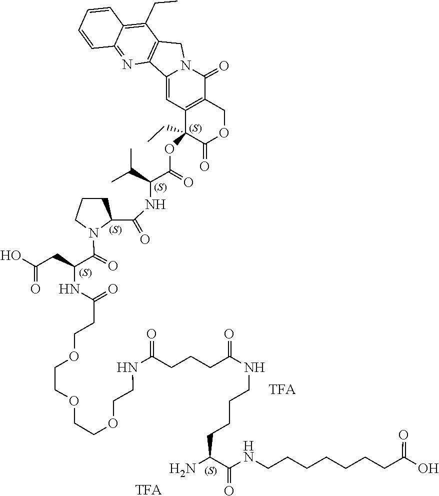

Step 1: To a solution of (4S)-4,11-diethyl-3,14-dioxo-3,4,12,14-tetrahydro-1H-pyrano[3′,4′:6,7]indolizino[1,2-b]quinolin-4-yl N-{18-[(2,5-dioxopyrrolidin-1-yl)oxy]-14,18-dioxo-4,7,10-trioxa-13-azaoctadecanan-1-oyl}-L-alpha-aspartyl-L-prolyl-L-valinate (65.0 mg, 93% purity, 54.6 μmol) (Intermediate 11) in DMF (2.5 ml) were added tert-butyl 8-{[N²-(tert-butoxycarbonyl)-L-lysyl]amino}octanoate (39.8 mg, 91% purity, 82.0 μmol) (Intermediate 51) and DIEA (38 μl, 220 μmol). The mixture was stirred at rt for 2 h and then concentrated under reduced pressure. The residue was purified by preparative HPLC and lyophilized to afford (4S)-4,11-diethyl-3,14-dioxo-3,4,12,14-tetrahydro-1H-pyrano[3′,4′:6,7]indolizino[1,2-b]quinolin-4-yl N-{(14S)-14-[(tert-butoxycarbonyl)amino]-2,2-dimethyl-4,13,20,24,37-pentaoxo-3,28,31,34-tetraoxa-12,19,25-triazaheptatriacontan-37-yl}-L-alpha-aspartyl-L-prolyl-L-valinate (66 mg, 92% purity, 78% yield) as a yellow foam. LC-MS (Method 3): Rₜ=4.92 min; MS (ESIpos): m/z=1431 [M+H]⁺

Step 2: To a solution of (4S)-4,11-diethyl-3,14-dioxo-3,4,12,14-tetrahydro-1H-pyrano[3′,4′:6,7]indolizino[1,2-b]quinolin-4-yl N-{(14S)-14-[(tert-butoxycarbonyl)amino]-2,2-dimethyl-4,13,20,24,37-pentaoxo-3,28,31,34-tetraoxa-12,19,25-triazaheptatriacontan-37-yl}-L-alpha-aspartyl-L-prolyl-L-valinate (65.0 mg, 92% purity, 41.8 μmol) in DCM (2.5 ml), was added TFA (1.2 ml). The mixture was stirred at rt for 1 h and then concentrated under reduced pressure. The residue was dissolved in ACN/H₂O and lyophilized to afford Intermediate 52 (78 mg, 94% purity, 117%) as an amorphous residue. LC-MS (Method 3): Rₜ=3.08 min; MS (ESIpos): m/z=1275 [M+H]⁺.

### Example S53: Preparation of (4S)-4,11-diethyl-3,14-dioxo-3,4,12,14-tetrahydro-1H-pyrano[3′,4′:6,7]indolizino[1,2-b]quinolin-4-yl N-{(24S)-37-amino-24-[(7-carboxyheptyl)carbamoyl]-14,18,26-trioxo-4,7,10,29,32,35-hexaoxa-13,19,25-triazaheptatriacontanan-1-oyl}-L-alpha-aspartyl-L-prolyl-L-valinate-trifluoroacetic acid (1/2) (Intermediate 53)

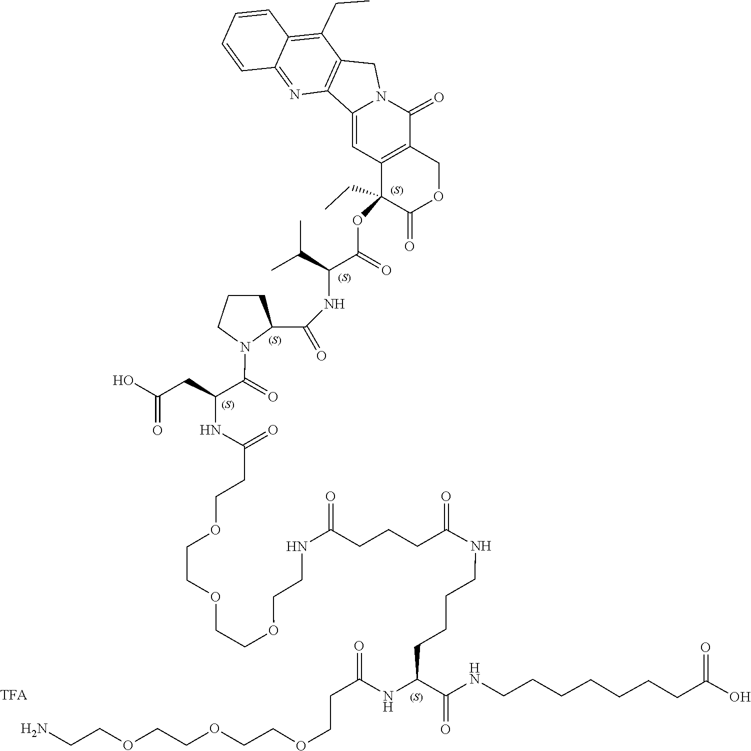

Step 1: To a solution of (4S)-4,11-diethyl-3,14-dioxo-3,4,12,14-tetrahydro-1H-pyrano[3′,4′:6,7]indolizino[1,2-b]quinolin-4-yl N-[(24S)-24-amino-33-carboxy-14,18,25-trioxo-4,7,10-trioxa-13,19,26-triazatritriacontanan-1-oyl]-L-alpha-aspartyl-L-prolyl-L-valinate-trifluoroacetic acid (1/2) (75.0 mg, 94% purity, 47.1 μmol) (Intermediate 52) in DMF (5 ml) was added tert-butyl {2-[2-(2-{3-[(2,5-dioxopyrrolidin-1-yl)oxy]-3-oxopropoxy}ethoxy)ethoxy]ethyl}carbamate (47.3 mg, 113 μmol) and DIEA (66 μl, 380 μmol). The mixture was stirred at rt for 1 h and then concentrated under reduced pressure. The residue was purified by preparative HPLC and lyophilized to afford (4S)-4,11-diethyl-3,14-dioxo-3,4,12,14-tetrahydro-1H-pyrano[3′,4′:6,7]indolizino[1,2-b]quinolin-4-yl N-{(19S)-19-[(7-carboxyheptyl)carbamoyl]-2,2-dimethyl-4,17,25,29,42-pentaoxo-3,8,11,14,33,36,39-heptaoxa-5,18,24,30-tetraazadotetracontan-42-yl}-L-alpha-aspartyl-L-prolyl-L-valinate (77 mg, 90% purity, 93% yield) as an amorphous residue. LC-MS (Method 3): Rₜ=4.10 min; MS (ESIpos): m/z=1578 [M+H]⁺.

Step 2: To a solution of (4S)-4,11-diethyl-3,14-dioxo-3,4,12,14-tetrahydro-1H-pyrano[3′,4′:6,7]indolizino[1,2-b]quinolin-4-yl N-{(19S)-19-[(7-carboxyheptyl)carbamoyl]-2,2-dimethyl-4,17,25,29,42-pentaoxo-3,8,11,14,33,36,39-heptaoxa-5,18,24,30-tetraazadotetracontan-42-yl}-L-alpha-aspartyl-L-prolyl-L-valinate (76.0 mg, 90% purity, 43.4 μmol) in DCM (2.5 ml), was added TFA (1.2 ml). The mixture was stirred at rt for 1 h and then concentrated under reduced pressure. The residue was dissolved in ACN/H₂O and lyophilized to afford Intermediate 53 (88 mg, 90% purity, 107%) as an amorphous residue. LC-MS (Method 3): Rₜ=3.10 min; MS (ESIpos): m/z=1478 [M+H]⁺.

### Example S54: Preparation of (3S)-3-{[(4-{[(4-nitrophenoxy)carbonyl]amino}phenyl)carbamoyl]amino}-3-{3-[({3-[(propylcarbamoyl)amino]phenyl}sulfonyl)amino]phenyl}propanoic acid (Intermediate 54)

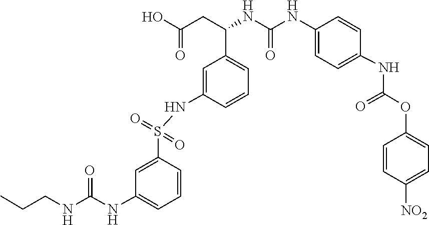 [SMILES: CCCNC(=O)Nc1cccc(S(=O)(=O)Nc2cccc([C@H](CC(=O)O)NC(=O)Nc3ccc(NC(=O)Oc4ccc([N+](=O)[O-])cc4)cc3)c2)c1]

The synthesis of Intermediate 54 has been described in WO2020/094471. LC-MS: Rₜ=0.89 min; MS (ESIpos): m/z=720 [M+H]⁺.

### Example S55: Preparation of N-{[methyl(2-{methyl[2-(methylamino)ethyl]amino}ethyl)amino]acetyl}-L-asparaginyl-L-prolyl-N-[2-(ethoxymethyl)-1-(2-hydroxy-2-methylpropyl)-1H-imidazo[4,5-c]quinolin-4-yl]-L-valinamide trifluoroacetate (1:1) (Intermediate 55)

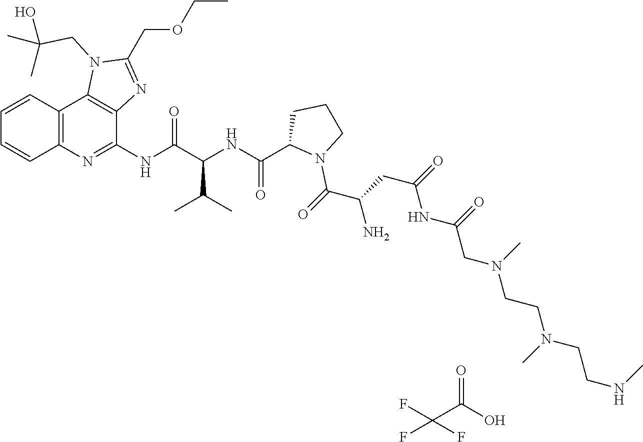 [SMILES: CCOCc1nc2c(NC(=O)[C@@H](NC(=O)[C@@H]3CCCN3C(=O)[C@@H](N)CC(=O)NC(=O)CN(C)CCN(C)CCNC)C(C)C)nc3ccccc3c2n1CC(C)(C)O ; O=C(O)C(F)(F)F]

Intermediate 56 (45.0 mg, 49.4 umol) was dissolved in DCM (8.0 ml). TFA (1.50 ml) was added and the reaction was stirred at RT for 30 min. The reaction was concentrated in vacuo, dissolved in ACN/H2O and lyophilised to give Intermediate 55 (45.0 mg, 100% purity, 99%/yield) as a white residue. LC-MS (Method 2): Rₜ=0.90 min; MS (ESIneg): m/z=808 [M−H]⁻.

### Example S56: Preparation of N-(2,2,5,8,11-pentamethyl-4,13-dioxo-3-oxa-5,8,11-triazatridecan-13-yl)-L-asparaginyl-L-prolyl-N-[2-(ethoxymethyl)-1-(2-hydroxy-2-methylpropyl)-1H-imidazo[4,5-c]quinolin-4-yl]-L-valinamide (Intermediate 56)

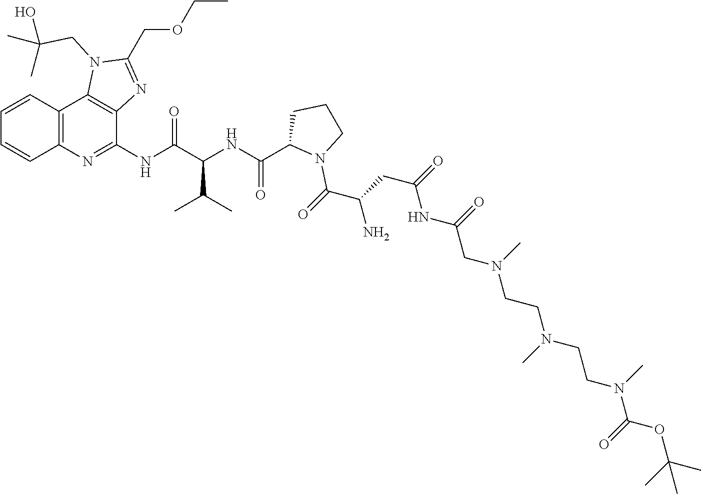 [SMILES: CCOCc1nc2c(NC(=O)[C@@H](NC(=O)[C@@H]3CCCN3C(=O)[C@@H](N)CC(=O)NC(=O)CN(C)CCN(C)CCN(C)C(=O)OC(C)(C)C)C(C)C)nc3ccccc3c2n1CC(C)(C)O]

Intermediate 58 (38.0 mg, 51.4 umol) was added to DMF (10.0 ml), then Intermediate 57 (30.7 mg, 61.7 umol), HATU (39.1 mg, 102.9 umol) and DIEA (35.8 ul, 205.7 umol) were added and the reaction was stirred overnight at RT. The reaction was then concentrated in vacuo. The residue was purified by prep HPLC, concentrated, and lyophilised to give Intermediate 56 (45.0 mg, 100% purity, 96% yield). LC-MS (Method 2): Rₜ=1.20 min; MS (ESIneg): m/z=908 [M−H]⁻.

### Example S57: Preparation of N-{2-[{2-[(tert-butoxycarbonyl)(methyl)amino]ethyl}(methyl)amino]ethyl}-N-methylglycine (Intermediate 57)

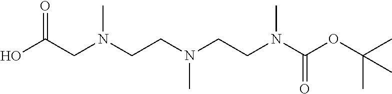 [SMILES: CN(CCN(C)CC(=O)O)CCN(C)C(=O)OC(C)(C)C]

Tert-butyl methyl(2-{methyl[2-(methylamino)ethyl]amino}ethyl)carbamate was obtained starting from commercially available N,N′-dimethyl-N-[2-(methylamino)ethyl]ethane-1,2-diamine.

Benzyl bromoacetate (609 mg, 2.66 mmol) was initially dissolved under argon in acetonitrile (25 mL) and potassium carbonate (734 mg, 5.31 mmol) was added. The solution was cooled to 0° C. and a solution of tert-butyl methyl(2-{methyl[2-(methylamino)ethyl]amino}ethyl)carbamate (652 mg, 2.66 mmol) in acetonitrile was added. The batch was stirred at rt for 1 h and subsequently filtered. The filtrate was evaporated in vacuo and the remaining residue was purified by prep. HPLC. Relevant fractions were collected and evaporated to dryness. 665 mg (97% purity, 62% yield) of the protected intermediate benzyl N-{2-[{2-[(tert-butoxycarbonyl)methyl)amino]ethyl}(methyl)amino]ethyl}-N-methylglycinate were obtained as a colorless oil. This intermediate (687 mg, 1.75 mmol) was dissolved in DCM/methanol and hydrogenated over 10% Pd on charcoal at rt for 2 h. The catalyst was filtered off and the filtrate was concentrated in vacuo to give Intermediate 57 (555 mg, 61% purity, 64% yield) as a colorless oil. LC-MS (Method 7): Rₜ=0.74 min; MS (ESIpos): m/z=304 [M+H]⁺.

### Example S58: Preparation of L-asparaginyl-L-prolyl-N-[2-(ethoxymethyl)-1-(2-hydroxy-2-methylpropyl)-1H-imidazo[4,5-c]quinolin-4-yl]-L-valinamide trifluoroacetate (1:1) (Intermediate 58)

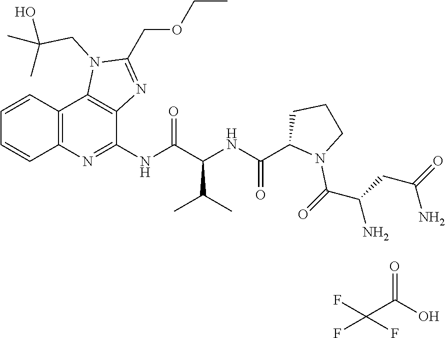

Intermediate 59 (40.0 mg, 0.051 mmol) was dissolved in DCM (10.0 ml), TFA (1.0 ml) was added, and the reaction was stirred at RT for 45 min. The reaction was concentrated in vacuo, the residue was dissolved in ACN/H2O and lyophilised to give Intermediate 58 (38.0 mg, 100% purity, quantitative yield) as a white residue. LC-MS (Method 2): 14=1.10 min; MS (ESIpos): m/z=632 [M+H]⁺.

### Example S59: Preparation of N²-(tert-butoxycarbonyl)-L-asparaginyl-L-prolyl-N-[2-(ethoxymethyl)-1-(2-hydroxy-2-methylpropyl)-1H-imidazo[4,5-c]quinolin-4-yl]-L-valinamide (Intermediate 59)

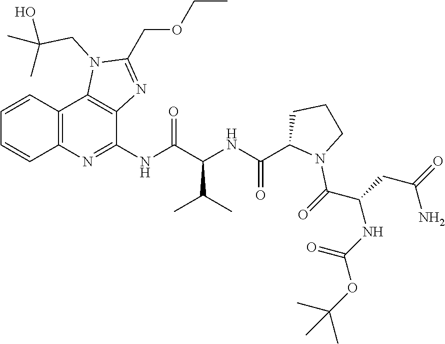 [SMILES: CCOCc1nc2c(NC(=O)[C@@H](NC(=O)[C@@H]3CCCN3C(=O)[C@H](CC(N)=O)NC(=O)OC(C)(C)C)C(C)C)nc3ccccc3c2n1CC(C)(C)O]

To a solution of Boc-Asn-OH (34.8 mg, 150.0 umol) in DMF (10.0 ml) were added HOBT (28.7 mg, 187.3 umol), EDCI (35.9 mg, 187.3 umol). The reaction was stirred for 10 min at RT, then Intermediate 60 (80.0 mg, 124.9 umol) and DIEA (65.2 ul, 374.6 umol) were added, and the reaction was stirred for 2 h at RT. The reaction was concentrated in vacuo. The residue was purified by prep HPLC and lyophilized to give Intermediate 59 (40.0 mg, 92% purity, 41% yield) as a white residue. LC-MS (Method 3): Rₜ=3.18 min; MS (ESIpos): m/z=725 [M+H]⁺.

### Example S60: Preparation of L-prolyl-N-[2-(ethoxymethyl)-1-(2-hydroxy-2-methylpropyl)-1H-imidazo[4,5-c]quinolin-4-yl]-L-valinamide trifluoroacetate (1:1) (Intermediate 60)

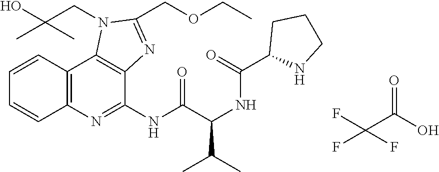 [SMILES: CCOCc1nc2c(NC(=O)[C@@H](NC(=O)[C@@H]3CCCN3)C(C)C)nc3ccccc3c2n1CC(C)(C)O ; O=C(O)C(F)(F)F]

Intermediate 61(178.5 mg, 254.9 mmol) was dissolved in DCM (12.0 ml), TFA (3.0 ml) was added and the reaction was stirred at RT for 30 min. The reaction was concentrated in vacuo. The residue was dissolved in ACN/H2O and lyophilized to give Intermediate 60 (165.0 mg, 98% purity, quantitative yield). LC-MS (Method 2): Rₜ=1.10 min; MS (ESIpos): m/z=509 [M−H]⁻.

### Example S61: Preparation of 1-(tert-butoxycarbonyl)-L-prolyl-N-[2-(ethoxymethyl)-1-(2-hydroxy-2-methylpropyl)-1H-imidazo[4,5-c]quinolin-4-yl]-L-valinamide (Intermediate 61)

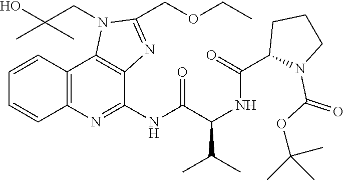 [SMILES: CCOCc1nc2c(NC(=O)[C@@H](NC(=O)[C@@H]3CCCN3C(=O)OC(C)(C)C)C(C)C)nc3ccccc3c2n1CC(C)(C)O]

Intermediate 62 (172.0 mg, 0.326 mmol) was added to DMF (10.0 ml). Boc-Pro-OH (77.2 mg, 0.359 mmol), HATU (186.0 mg, 489.1 umol) and DIEA (126.4 mg, 978.1 umol) were added, and the reaction was stirred for 30 min at RT. The reaction was concentrated in vacuo and the residue was purified by prep HPLC, concentrated, and lyophilised to give Intermediate 61 (178.0 mg, 87% purity, 78% yield) as a white foam. LC-MS (Method 2): Rₜ=1.65 min; MS (ESIpos): m/z=611 [M+H]⁺.

### Example S62: Preparation of N-[2-(ethoxymethyl)-1-(2-hydroxy-2-methylpropyl)-1H-imidazo[4,5-c]quinolin-4-yl]-L-valinamide trifluoroacetate (1:1) (Intermediate 62)

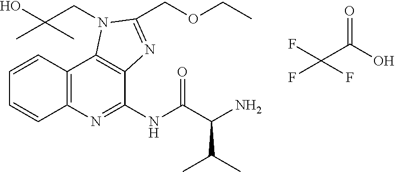 [SMILES: CCOCc1nc2c(NC(=O)[C@@H](N)C(C)C)nc3ccccc3c2n1CC(C)(C)O ; O=C(O)C(F)(F)F]

Intermediate 63 (160 mg, 311.7 umol) was dissolved in DCM (10.0 ml), TFA (2.0 ml) was added, and the reaction was stirred at RT for 1.5h. The reaction was concentrated in vacuo, dissolved in ADC/H2O and lyophilized to give Intermediate 62 (172 mg, 100% purity, 67% yield) as a white foam. LC-MS (Method 2): Rₜ=0.99 min; MS (ESIpos): m/z=414 [M+H]⁺.

### Example S63: Preparation of tert-butyl [(2S)-1-{[2-(ethoxymethyl)-1-(2-hydroxy-2-methylpropyl)-1H-imidazo[4,5-c]quinolin-4-yl]amino}-3-methyl-1-oxobutan-2-yl]carbamate (Intermediate 63)

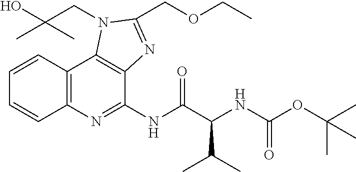 [SMILES: CCOCc1nc2c(NC(=O)[C@@H](NC(=O)OC(C)(C)C)C(C)C)nc3ccccc3c2n1CC(C)(C)O]

1-(4-Amino-2-(ethoxymethyl)-1H (Resiquimod) (80.0 mg, 0.254 mmol) was added to DCM (15.0 ml). Then, Boc-Val-NCA (123.8 mg, 0.509 mmol) and DMAP (31.1 mg, 0.254 mmol) were added. The mixture was heated and was stirred under reflux for 2 h. The reaction was concentrated in vacuo and the residue was purified by prep. HPLC, concentrated, and lyophilized to give Intermediate 63 (160 mg, 96% purity, quantitative yield) as a white foam. LC-MS (Method 2): Rₜ=1.64 min; MS (ESIpos): m/z=514 [M+H]⁺.

### Example S64: Preparation of N-{14-[4-({[(1R)-2-carboxy-1-{3-[({3-[(propylcarbamoyl)amino]phenyl}sulfonyl)amino]phenyl}ethyl]carbamoyl}amino)anilino]-14-oxo-4,7,10-trioxa-13-azatetradecan-1-oyl}-L-alpha-aspartyl-L-prolyl-N-[2-(ethoxymethyl)-1-(2-hydroxy-2-methylpropyl)-1H-imidazo[4,5-c]quinolin-4-yl]-L-valinamide (Intermediate 64)

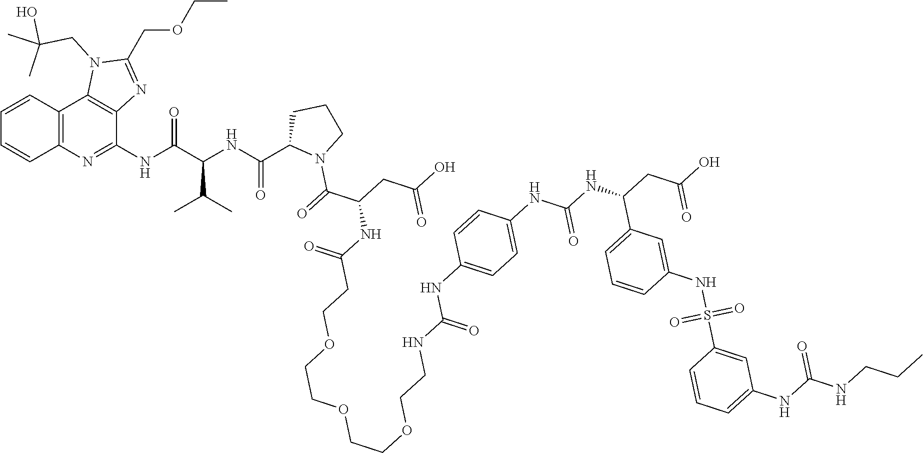 [SMILES: CCCNC(=O)Nc1cccc(S(=O)(=O)Nc2cccc([C@@H](CC(=O)O)NC(=O)Nc3ccc(NC(=O)NCCOCCOCCOCCC(=O)N[C@@H](CC(=O)O)C(=O)N4CCC[C@H]4C(=O)N[C@H](C(=O)Nc4nc5ccccc5c5c4nc(COCC)n5CC(C)(C)O)C(C)C)cc3)c2)c1]

Intermediate 65 (60.9 mg, 64.6 umol) was dissolved in DMF (10.0 ml). Intermediate 2 (46.5 mg, 64.6 umol) and DIEA (225.0 ul, 1291.6 umol) were added. The reaction was stirred for 15 minutes at RT and was then concentrated in vacuo. The residue was separated by prep. HPLC to give Intermediate 64 (58.0 mg, 97% purity, 62% yield) as a colorless foam. LC-MS (Method 2): Rₜ=1.40 min; MS (ESIneg): m/z=1407 [M−H]⁻.

### Example S65: Preparation of N-(3-{2-[2-(2-aminoethoxy)ethoxy]ethoxy}propanoyl)-L-alpha-aspartyl-L-prolyl-N-[2-(ethoxymethyl)-1-(2-hydroxy-2-methylpropyl)-1H-imidazo[4,5-c]quinolin-4-yl]-L-valinamide trifluoroacetate (1:1) (Intermediate 65)

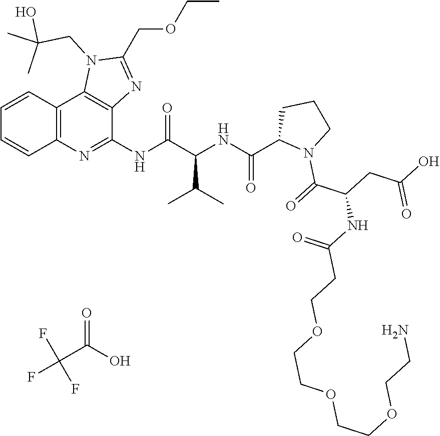 [SMILES: CCOCc1nc2c(NC(=O)[C@@H](NC(=O)[C@@H]3CCCN3C(=O)[C@H](CC(=O)O)NC(=O)CCOCCOCCOCCN)C(C)C)nc3ccccc3c2n1CC(C)(C)O ; O=C(O)C(F)(F)F]

Intermediate 66 (58.8 mg, 63.3 umol) was dissolved in DCM (5.0 ml). TFA (1.0 ml) was added and the reaction was stirred for 30 min at rt. The reaction was concentrated on an oil pump and the residue was dissolved in ACN/H2O and lyophilised to give Intermediate 65 (60.9 mg, 100% purity, quantitative yield) as a colorless foam. LC-MS (Method 2): Rₜ=0.96 min; MS (ESIneg): m/z=827 [M−H]⁻.

### Example S66: Preparation of N-(2,2-dimethyl-4,17-dioxo-3,8,11,14-tetraoxa-5-azaheptadecan-17-yl)-L-alpha-aspartyl-L-prolyl-N-[2-(ethoxymethyl)-1-(2-hydroxy-2-methylpropyl)-1H-imidazo[4,5-c]quinolin-4-yl]-L-valinamide (Intermediate 66)

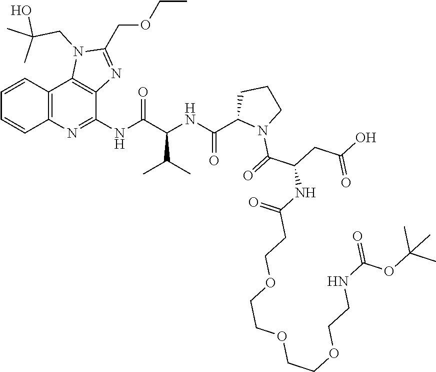 [SMILES: CCOCc1nc2c(NC(=O)[C@@H](NC(=O)[C@@H]3CCCN3C(=O)[C@H](CC(=O)O)NC(=O)CCOCCOCCOCCNC(=O)OC(C)(C)C)C(C)C)nc3ccccc3c2n1CC(C)(C)O]

Intermediate 67 (69.0 mg, 0.093 mmol) was dissolved in DMF (8.0 ml). tBoc-N-amido-PEG3-NHS ester (46.8 mg, 0.112 mmol) and DIEA (48.7 ul, 279.8 umol) were added. The reaction was stirred at RT for 3 h. The reaction was evaporated to dryness and the residue was separated by prep. HPLC to give Intermediate 66 (58.8 mg, 100% purity, 68% yield) as a colorless foam. LC-MS (Method 2): Rₜ=1.45 min; MS (ESIpos): m/z=929 [M+H]⁺.

### Example S67: Preparation of L-alpha-aspartyl-L-prolyl-N-[2-(ethoxymethyl)-1-(2-hydroxy-2-methylpropyl)-1H-imidazo[4,5-c]quinolin-4-yl]-L-valinamide-trifluoroacetic acid (1/1) (Intermediate 67)

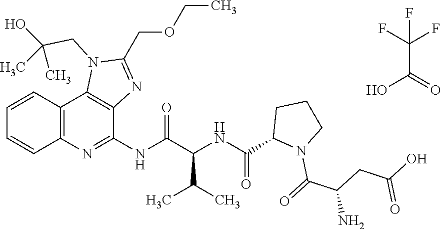 [SMILES: CCOCc1nc2c(NC(=O)[C@@H](NC(=O)[C@@H]3CCCN3C(=O)[C@@H](N)CC(=O)O)C(C)C)nc3ccccc3c2n1CC(C)(C)O ; O=C(O)C(F)(F)F]

Intermediate 68 (200 mg, 235 μmol) was dissolved in DCM (15 ml) and TFA (5.0 ml) was added. The reaction was stirred at RT for 4.5 h and then concentrated in vacuo. The residue was dissolved in ACN/H2O and lyophilised. The residue was again redissolved in DCM/MeOH, ether was added until cloudy. The emulsion was cooled briefly then decanted. The remaining residue was dissolved in ACN/H2O and lyophilised again to give Intermediate 67 (148 mg, 85% yield). LC-MS (Method 8): Rₜ=0.53 min; MS (ESIneg): m/z=624 [M−H]⁻.

### Example S68: Preparation of tert-butyl (3S)-3-[(tert-butoxycarbonyl)amino]-4-[(2S)-2-{[(2S)-1-{[2-(ethoxymethyl)-1-(2-hydroxy-2-methylpropyl)-1H-imidazo[4,5-c]quinolin-4-yl]amino}-3-methyl-1-oxobutan-2-yl]carbamoyl}pyrrolidin-1-yl]-4-oxobutanoate (Intermediate 68)

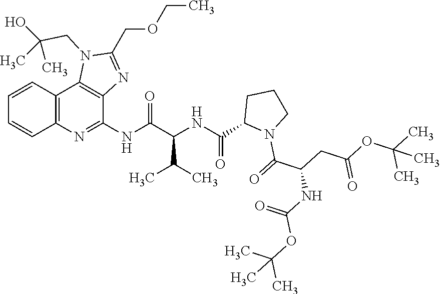 [SMILES: CCOCc1nc2c(NC(=O)[C@@H](NC(=O)[C@@H]3CCCN3C(=O)[C@H](CC(=O)OC(C)(C)C)NC(=O)OC(C)(C)C)C(C)C)nc3ccccc3c2n1CC(C)(C)O]

Intermediate 62 (160 mg, 303 μmol), Intermediate 69 (141 mg, 364 μmol), HATU (150 mg, 394 μmol) and DIEA (160 μl, 910 μmol) were dissolved in DMF (15 ml) and stirred at RT for 10 min. The reaction was concentrated in vacuo and purified by prep HPLC to give Intermediate 68 (200 mg, 92% purity, 77% yield). LC-MS (Method 2): Rₜ=1.89 min; MS (ESIpos): m/z=782 [M+H]⁺.

### Example S69: Preparation of (2S)-1-((2S)-4-tert-butoxy-2-[(tert-butoxycarbonyl)amino]-4-oxobutanoyl)pyrrolidine-2-carboxylic acid (Intermediate 69)

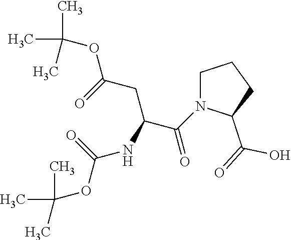 [SMILES: CC(C)(C)OC(=O)C[C@H](NC(=O)OC(C)(C)C)C(=O)N1CCC[C@H]1C(=O)O]

Intermediate 70 (7.47 g, 15.7 mmol) was dissolved in methanol (1.0 L) and Pd/C 10% (1.30g) was added. The reaction was hydrogenated at RT, filtered, concentrated, and extracted with ether. The compound was dried on the high vacuum line to give Intermediate 69 (5.92 g, 97% purity, 95% yield) as a foam. LC-MS (Method 2): Rt=1.61 min; MS (ESIpos): m/z=387 [M+H]⁺.

### Example S70: Preparation of benzyl (2S)-1-((2S)-4-tert-butoxy-2-[(tert-butoxycarbonyl)amino]-4-oxobutanoyl)pyrrolidine-2-carboxylate (Intermediate 70)

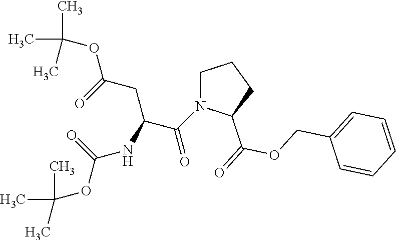 [SMILES: CC(C)(C)OC(=O)C[C@H](NC(=O)OC(C)(C)C)C(=O)N1CCC[C@H]1C(=O)OCc1ccccc1]

Boc-Asp(OtBu)-OSu (0.390 g, 1.01 mmol) was dissolved in DMF (10 ml). H-Pro-OBzl·HCl (0.244 g, 1.01 mmol) and DIEA (0.352 ml, 2.02 mmol) were added and the reaction was stirred at RT overnight. The reaction was concentrated in vacuo, dissolved in DCM and extracted with H2O. The organic phase was purified by flash column chromatography (DCM/3% MeOH). The product was concentrated in vacuo and dried on the high vacuum line to give Intermediate 70 (0.41 g, 100% purity, 85% yield). LC-MS (Method 2): Rₜ=2.20 min; MS (ESIpos): m/z=477 [M+H]⁺.

### Example S71 Preparation of N-[(2S)-4-[{(1R)-1-[1-benzyl-4-(2,5-difluorophenyl)-1H-pyrrol-2-yl]-2,2-dimethylpropyl}(glycoloyl)amino]-1-(methylamino)-1-oxobutan-2-yl]-L-valinamide trifluoroacetate (1:1) (Intermediate 71)

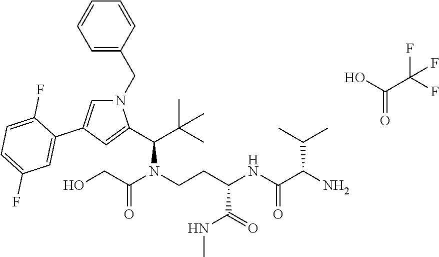 [SMILES: CNC(=O)[C@H](CCN(C(=O)CO)[C@@H](c1cc(-c2cc(F)ccc2F)cn1Cc1ccccc1)C(C)(C)C)NC(=O)[C@@H](N)C(C)C ; O=C(O)C(F)(F)F]

Step 1: (2S)-2-amino-4-[{(1R)-1-[1-benzyl-4-(2,5-difluorophenyl)-1H-pyrrol-2-yl]-2,2-dimethylpropyl}(glycoloyl)amino]-N-methylbutanamide trifluoroacetate (1:1) (100.0 mg, 0.155 mmol), the synthesis of which is described in WO2015096982, was dissolved in DMF (2.5 ml). N-(tert-butoxycarbonyl)valine (40.5 mg, 0.187 mmol), HATU (88.7 mg, 0.233 mmol) and DIEA (81.2 ul, 0.466 mmol) were added and the reaction was stirred at RT for 3.5h. The residues was purified by prep HPLC and then lyophilised to give tert-butyl N-[(1S)-1-[[(1S)-3-[[(1R)-1-[1-benzyl-4-(2,5-difluorophenyl)pyrrol-2-yl]-2,2-dimethyl-propyl]-(2-hydroxyacetyl)amino]-1-(methylcarbamoyl)propyl]carbamoyl]-2-methyl-propyl]carbamate (99.4 mg, 100% purity, 88% yield). LC-MS (Method 2): Rt=2.43 min; MS (ESIpos): m/z=724 [M−H]⁻.

Step 2: tert-butyl N-[(1S)-1-[[(1S)-3-[[(1R)-1-[1-benzyl-4-(2,5-difluorophenyl)pyrrol-2-yl]-2,2-dimethyl-propyl]-(2-hydroxyacetyl)amino]-1-(methylcarbamoyl)propyl]carbamoyl]-2-methyl-propyl]carbamate (99.2 mg, 0.137 mmol) was dissolved in 2,2,2-trifluoroethanol (4.0 ml). ZnCl₂ (111.8 mg, 0.82 mmol) was added and the reaction was stirred for 2 h at 50 C. EDTA (239.6 mg, 0.82 mmol) and H₂O+0.1% TFA (2 ml) were added and the residue was purified by prep HPLC and then lyophilised to give Intermediate 71(94.6 mg, 100% purity, 94% yield). LC-MS (Method 2): Rt=1.53 min; MS (ESIpos): m/z=626 [M+H].

### Example S72 Preparation of N-(3-{2-[2-(2-aminoethoxy)ethoxy]ethoxy}propanoyl)-L-alpha-aspartyl-L-prolyl-N-[(2S)-4-[{(1R)-1-[1-benzyl-4-(2,5-difluorophenyl)-1H-pyrrol-2-yl]-2,2-dimethylpropyl}(glycoloyl)amino]-1-(methylamino)-1-oxobutan-2-yl]-L-valinamide trifluoroacetate (1:1)) (Intermediate 72)

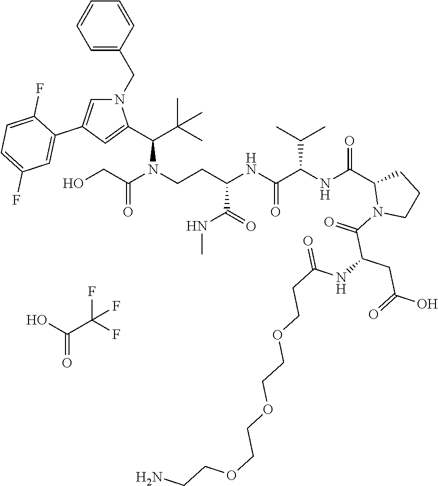 [SMILES: CNC(=O)[C@H](CCN(C(=O)CO)[C@@H](c1cc(-c2cc(F)ccc2F)cn1Cc1ccccc1)C(C)(C)C)NC(=O)[C@@H](NC(=O)[C@@H]1CCCN1C(=O)[C@H](CC(=O)O)NC(=O)CCOCCOCCOCCN)C(C)C ; O=C(O)C(F)(F)F]

Step 1: To a solution of Intermediate 71 (94.4 mg, 0.128 mmol) and Intermediate 3 (82.8 mg, 0.14 mmol) in DMF (3.0 ml) were added HATU (77.6 mg, 0.204 mmol) and DIEA (66.7 ul, 0.383 mmol). The reaction was stirred at RT for 1.5h. The residue was purified by prep HPLC and then lyophilised to give tert-butyl (19S)-19-{[(2S)-2-{[(2S)-1-{[(2S)-4-[{(1R)-1-[1-benzyl-4-(2,5-difluorophenyl)-1H-pyrrol-2-yl]-2,2-dimethylpropyl}(glycoloyl)amino]-1-(methylamino)-1-oxobutan-2-yl]amino}-3-methyl-1-oxobutan-2-yl]carbamoyl}pyrrolidin-1-yl]carbonyl}-2,2-dimethyl-4,17-dioxo-3,8,11,14-tetraoxa-5,18-diazahenicosan-21-oate (143.7 mg, 100% purity, 94% yield). LC-MS (Method 2): Rt=2.41 min; MS (ESIpos): m/z=1197 [M+H]⁺.

Step 2: tert-butyl (19S)-19-{[(2S)-2-{[(2S)-1-{[(2S)-4-[{(1R)-1-[1-benzyl-4-(2,5-difluorophenyl)-1H-pyrrol-2-yl]-2,2-dimethylpropyl}(glycoloyl)amino]-1-(methylamino)-1-oxobutan-2-yl]amino}-3-methyl-1-oxobutan-2-yl]carbamoyl}pyrrolidin-1-yl]carbonyl}-2,2-dimethyl-4,17-dioxo-3,8,11,14-tetraoxa-5,18-diazahenicosan-2I-oate (143.4 mg, 0.120 mmol) was dissolved in 2,2,2-trifluoroethanol (4.0 ml). ZnCl₂ (97.9 mg, 0.719 mmol) was added and the reaction was stirred for 2 h at 50 C. 3 further equivalents of ZnCl₂ were added and the reaction was stirred for 1 h at 50 C. EDTA (210.0 mg, 0.719 mmol) and H20+0.1% TFA (2 ml) were added. The residue was purified by prep HPLC and then lyophilised to give Intermediate 72 (125.6 mg, 100% purity, 91% yield). LC-MS (Method 2): Rt=1.54 min; MS (ESIpos): m/z=1041 [M+H]⁺.

### Example S73 Preparation of (3S)-3-[3-[2-[2-(2-aminoethoxy)ethoxy]ethoxy]propanoylamino]-4-[(2S)-2-[[(1S)-1-[3-[[(1R)-1-[1-benzyl-4-(2,5-difluorophenyl)pyrrol-2-yl]-2,2-dimethyl-propyl]-(2-hydroxyacetyl)amino]propylcarbamoyl]-2-methyl-propyl]carbamoyl]pyrrolidin-1-yl]-4-oxo-butanoic acid; 2,2,2-trifluoroacetic acid (Intermediate 73)

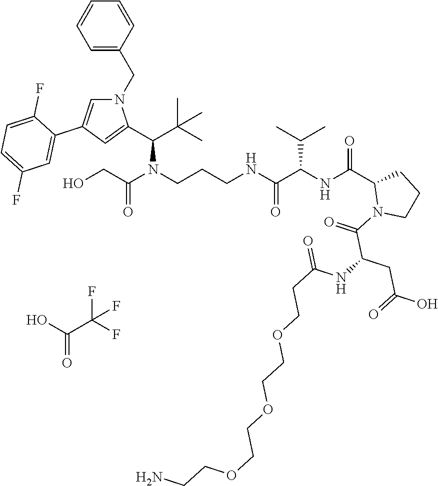 [SMILES: CC(C)[C@H](NC(=O)[C@@H]1CCCN1C(=O)[C@H](CC(=O)O)NC(=O)CCOCCOCCOCCN)C(=O)NCCCN(C(=O)CO)[C@@H](c1cc(-c2cc(F)ccc2F)cn1Cc1ccccc1)C(C)(C)C ; O=C(O)C(F)(F)F]

Step 1: To a solution of Intermediate 41(100 mg, 0.143 mmol) and Intermediate 3 (100.9 mg, 0.171 mmol) in DMF (20 ml) were added HATU (70.4 mg, 0.185 mmol) and DIEA (74.5 ul, 0.428 mmol). The reaction was stirred at RT for 1 h. The solvent was evaporated and the residue was purified by prep HPLC and then lyophilized to give tert-butyl (3S)-4-[(2S)-2-[[(1S)-1-[3-[[(1R)-1-[1-benzyl-4-(2,5-difluorophenyl)pyrrol-2-yl]-2,2-dimethyl-propyl]-(2-hydroxyacetyl)amino]propylcarbamoyl]-2-methyl-propyl]carbamoyl]pyrrolidin-1-yl]-3-[3-[2-[2-[2-(tert-butoxycarbonylamino)ethoxy]ethoxy]ethoxy]propanoylamino]-4-oxo-butanoate (164 mg, 99/a purity, 100% yield). LC-MS (Method 3): Rt=5.87 min; MS (ESIpos): m/z=1141 [M+H]⁺.

Step 2: tert-butyl (3S)-4-[(2S)-2-[[(1S)-1-[3-[[(1R)-1-[1-benzyl-4-(2,5-difluorophenyl)pyrrol-2-yl]-2,2-dimethyl-propyl]-(2-hydroxyacetyl)amino]propylcarbamoyl]-2-methyl-propyl]carbamoyl]pyrrolidin-1-yl]-3-[3-[2-[2-[2-(tert-butoxycarbonylamino)ethoxy]ethoxy]ethoxy]propanoylamino]-4-oxo-butanoate (25 mg, 0.022 mmol) was dissolved in 2,2,2-trifluoroethanol (5 ml). ZnCl₂ (23.9 mg, 0.175 mmol) was added and the reaction was stirred for 5h at 50 C. EDTA (51.2 mg, 0.175 mmol) and water+0.1% TFA (3 ml) were added. The mixture was purified by prep HPLC and then lyophilised to give Intermediate 73 (20 mg, 98% purity, 81% yield) as an amorphous residue. LC-MS (Method 3): Rt=3.89 min; MS (ESIpos): m/z=984 [M+H]⁺.

### Example S74: Preparation of N-[3-(2-{2-[2-(L-lysylamino)ethoxy]ethoxy}ethoxy)propanoyl]-L-alpha-aspartyl-L-prolyl-N-[(2S)-4-[{(1R)-1-[1-benzyl-4-(2,5-difluorophenyl)-1H-pyrrol-2-yl]-2,2-dimethylpropyl}(hydroxyacetyl)amino]-1-(methylamino)-1-oxobutan-2-yl]-L-valinamide (Intermediate 74)

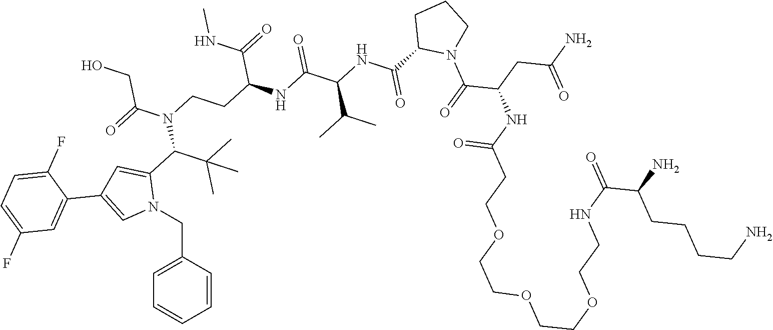 [SMILES: CNC(=O)[C@H](CCN(C(=O)CO)[C@@H](c1cc(-c2cc(F)ccc2F)cn1Cc1ccccc1)C(C)(C)C)NC(=O)[C@@H](NC(=O)[C@@H]1CCCN1C(=O)[C@H](CC(N)=O)NC(=O)CCOCCOCCOCCNC(=O)[C@@H](N)CCCCN)C(C)C]

Step 1: N-(3-{2-[2-(2-aminoethoxy)ethoxy]ethoxy}propanoyl)-L-alpha-aspartyl-L-prolyl-N-[(2S)-4-[{(1R)-1-[1-benzyl-4-(2,5-difluorophenyl)-1H-pyrrol-2-yl]-2,2-dimethylpropyl}(hydroxyacetyl)amino]-1-(methylamino)-1-oxobutan-2-yl]-L-valinamide.trifluoroacetic acid (1/1) (355 mg, 307 μmol) (Intermediate 72) was dissolved in 12 mL DMF and 2,5-dioxopyrrolidin-1-yl N²,N⁶-bis[(benzyloxy)carbonyl]-L-lysinate (215 mg, 95% purity, 399 μmol) as well as 110 μL N,N-diisopropylethylamine were added. After stirring for 4 h at rt, the mixture was concentrated in vacuo and the residue was purified by preparative HPLC to yield N-[3-(2-{2-[2-({N2,N6-bis[(benzyloxy)carbonyl]-L-lysyl}amino)ethoxy]ethoxy}ethoxy)propanoyl]-L-alpha-aspartyl-L-prolyl-N-[(2S)-4-[{(1R)-1-[1-benzyl-4-(2,5-difluorophenyl)-1H-pyrrol-2-yl]-2,2-dimethylpropyl}(hydroxyacetyl)amino]-1-(methylamino)-1-oxobutan-2-yl]-L-valinamide (412 mg, 100% purity, 93% yield) as white foam. LC-MS (Method 2): Rₜ=5.35 min; MS (ESIpos): m/z=1438 [M+H]⁺

Step 2: To a solution of N-[3-(2-{2-[2-({N²,N⁶-bis[(benzyloxy)carbonyl]-L-lysyl}amino)ethoxy]ethoxy}ethoxy)propanoyl]-L-alpha-aspartyl-L-prolyl-N-[(2S)-4-[{(1R)-1-[1-benzyl-4-(2,5-difluorophenyl)-1H-pyrrol-2-yl]-2,2-dimethylpropyl}(hydroxyacetyl)amino]-1-(methylamino)-1-oxobutan-2-yl]-L-valinamide (410 mg, 285 μmol) in DCM (10 ml) and methanol (10 ml) was added Pd/C (40 mg). The reaction was hydrogenated at rt for 4 h and filtered over celite. The mother liquor was concentrated in vacuo to afford Intermediate 74 (354 mg, 100% purity, quant.). LC-MS (Method 2): Rₜ=1.28 min; MS (ESIpos): m/z=1168 [M−H]⁻.

### Example S75 Preparation of N-(3-{2-[2-(2-{[N2,N6-bis(3-{2-[2-(2-aminoethoxy)ethoxy]ethoxy}propanoyl)-L-lysyl]amino}ethoxy)ethoxy]ethoxy}propanoyl)-L-alpha-aspartyl-L-prolyl-N-[(2S)-4-[{(1R)-1-[1-benzyl-4-(2,5-difluorophenyl)-1H-pyrrol-2-yl]-2,2-dimethylpropyl}(hydroxyacetyl)amino]-1-(methylamino)-1-oxobutan-2-yl]-L-valinamide.trifluoroacetic acid (1/2) (Intermediate 75)

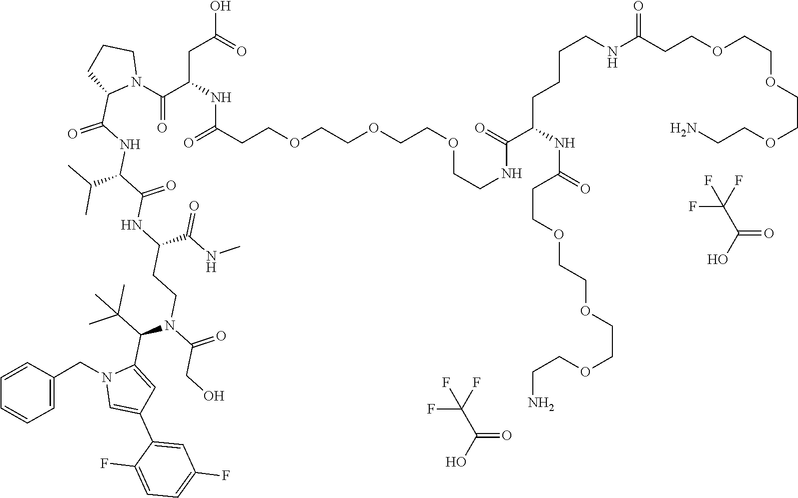

Step 1: N-[3-(2-{2-[2-(L-lysylamino)ethoxy]ethoxy}ethoxy)propanoyl]-L-alpha-aspartyl-L-prolyl-N-[(2S)-4-[{(1R)-1-[1-benzyl-4-(2,5-difluorophenyl)-1H-pyrrol-2-yl]-2,2-dimethylpropyl}(hydroxyacetyl)amino]-1-(methylamino)-1-oxobutan-2-yl]-L-valinamide (352 mg, 301 μmol) (Intermediate 74) was dissolved in 12 mL DMF and 2.2 eq tert-butyl {2-[2-(2-{3-[(2,5-dioxopyrrolidin-1-yl)oxy]-3-oxopropoxy}ethoxy)ethoxy]ethyl}carbamate (302 mg, 722 μmol) as well as 310 μL N,N-diisopropylethylamine were added. After stirring for 2h 30 at rt the mixture was concentrated in vacuo and the residue was purified by preparative HPLC and lyophilized to yield N-(3-{2-[2-(2-{[N²,N⁶-bis(2,2-dimethyl-4,17-dioxo-3,8,11,14-tetraoxa-5-azaheptadecan-17-yl)-L-lysyl]amino}ethoxy)ethoxy]ethoxy}propanoyl)-L-alpha-aspartyl-L-prolyl-N-[(2S)-4-[{(1R)-1-[1-benzyl-4-(2,5-difluorophenyl)-1H-pyrrol-2-yl]-2,2-dimethylpropyl}(hydroxyacetyl)amino]-1-(methylamino)-1-oxobutan-2-yl]-L-valinamide (449 mg, 100% purity, 84% yield) as a white foam. LC-MS (Method 9): Rₜ=9.77 min; MS (ESIpos): m/z=1177 [M+H]⁺

Step 2: To a solution of N-(3-{2-[2-(2-{[N2,N6-bis(2,2-dimethyl-4,17-dioxo-3,8,11,14-tetraoxa-5-azaheptadecan-17-yl)-L-lysyl]amino}ethoxy)ethoxy]ethoxy}propanoyl)-L-alpha-aspartyl-L-prolyl-N-[(2S)-4-[{(1R)-1-[1-benzyl-4-(2,5-difluorophenyl)-1H-pyrrol-2-yl]-2,2-dimethylpropyl}(hydroxyacetyl)amino]-1-(methylamino)-1-oxobutan-2-yl]-L-valinamide (447 mg, 252 μmol) in Trifluoroethanol (8.0 ml), was added zinc chloride (206 mg, 1.51 mmol). The mixture was stirred at 50° C. for 3 h. The resulting mixture was diluted in water and EDTA (441 mg, 1.51 mmol) was then added. The resulting mixture was filtered, purified over preparative HPLC and lyophilized to afford Intermediate 75 (371 mg, 100% purity, 82% yield) as a white foam. LC-MS (Method 3): R₄=3.18 min; MS (ESIpos): m/z=1576 [M+H]⁺

### Example S76 Preparation of (4S)-4,11-diethyl-3,14-dioxo-3,4,12,14-tetrahydro-1H-pyrano[3′,4′:6,7]indolizino[1,2-b]quinolin-4-yl N-[14-(4-nitrophenoxy)-14-oxo-4,7,10-trioxa-13-azatetradecanan-1-oyl]-L-alpha-aspartyl-L-prolyl-L-valinate (Intermediate 76)

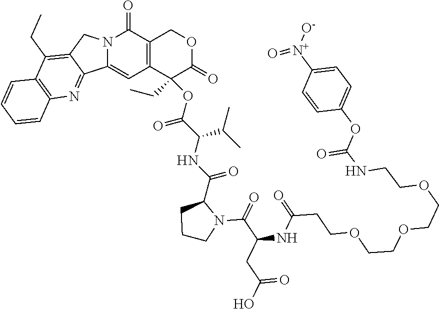 [SMILES: CCc1c2c(nc3ccccc13)-c1cc3c(c(=O)n1C2)COC(=O)[C@@]3(CC)OC(=O)[C@@H](NC(=O)[C@@H]1CCCN1C(=O)[C@H](CC(=O)O)NC(=O)CCOCCOCCOCCNC(=O)Oc1ccc([N+](=O)[O-])cc1)C(C)C]

To a solution of (4S)-4,11-diethyl-3,14-dioxo-3,4,12,14-tetrahydro-1H-pyrano[3,4′:6,7]indolizino[1,2-b]quinolin-4-yl N-(3-{2-[2-(2-aminoethoxy)ethoxy]ethoxy}propanoyl)-L-alpha-aspartyl-L-prolyl-L-valinate.trifluoroacetic acid (1/1) (20.0 mg, 19.9 μmol) Intermediate 6 in THF (5.0 ml) was added 4-nitrophenyl carbonochloridate (6.42 mg, 31.8 μmol). The mixture was stirred at rt for 2 h and then concentrated under reduced pressure. The residue was purified over preparative HPLC and freeze dried to afford Intermediate 76 (6.0 mg, 100% purity, 29% yield) as a colorless foam. LC-MS (Method 2): Rₜ=1.77 min; MS (ESIpos): m/z=1056 [M−H]⁻.

### Example S77 Preparation of methyl (3S)-3-[4-[4-[2-[2-[2-(2-azidoethoxy)ethoxy]ethoxy]ethoxy]-1-naphthyl]phenyl]-3-[[2-[5-[(4-methyl-2-pyridyl)amino]pentanoylamino]acetyl]amino]propanoate (Intermediate 77)

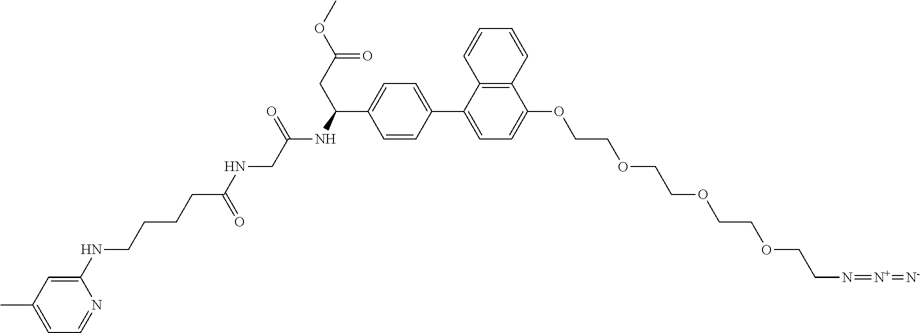 [SMILES: COC(=O)C[C@H](NC(=O)CNC(=O)CCCCNc1cc(C)ccn1)c1ccc(-c2ccc(OCCOCCOCCOCCN=[N+]=[N-])c3ccccc23)cc1]

Intermediate 77 was prepared using the procedure described in WO2022056273 for the synthesis of methyl (3S)-3-[4-[4-[2-[2-[2-[2-(2-azidoethoxy)ethoxy]ethoxy]ethoxy]ethoxy]-1-naphthyl]phenyl]-3-[[2-[5-[(4-methyl-2-pyridyl)amino]pentanoylamino]acetyl]amino]propanoate (CAS Nr: 2763952-63-0).

### Example S78 Preparation of (3S)-3-[4-[4-[2-[2-[2-(2-aminoethoxy)ethoxy]ethoxy]ethoxy]-1-naphthyl]phenyl]-3-[[2-[5-[(4-methyl-2-pyridyl)amino]pentanoylamino]acetyl]amino]propanoic acid (Intermediate 78)

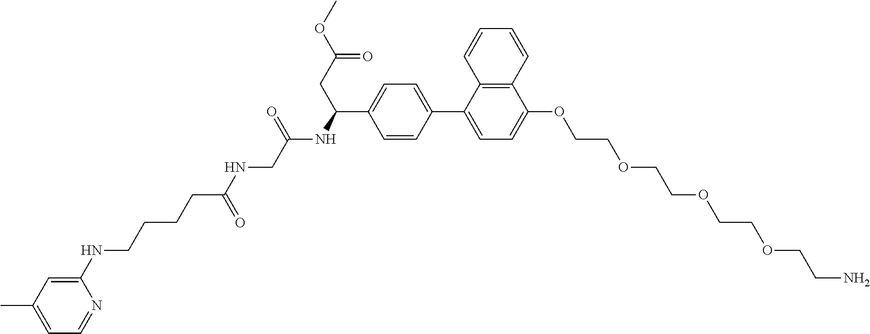 [SMILES: COC(=O)C[C@H](NC(=O)CNC(=O)CCCCNc1cc(C)ccn1)c1ccc(-c2ccc(OCCOCCOCCOCCN)c3ccccc23)cc1]

Intermediate 78 was prepared starting from Intermediate 77 by reduction of the azide using triphenylphosphine in a mixture of THF and water followed by saponification of the methyl ester using Lithium Hydroxide in a mixture of THF and water. LC-MS (Method 2): Rt=0.90 min; MS: m/z=728 [M−H]⁻.

### Example S79 Preparation of (3S)-3-[[(2S)-3-amino-2-[5-[tert-butoxycarbonyl-(4-methyl-2-pyridyl)amino]pentanoylamino]propanoyl]amino]-3-(3,5-dichlorophenyl)propanoic acid (Intermediate 79)

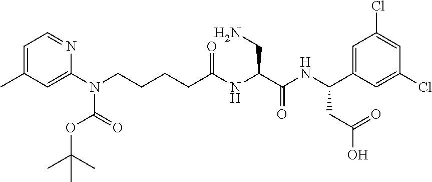 [SMILES: Cc1ccnc(N(CCCCC(=O)N[C@@H](CN)C(=O)N[C@@H](CC(=O)O)c2cc(Cl)cc(Cl)c2)C(=O)OC(C)(C)C)c1]

Intermediate 79 was prepared using the procedure described in WO2015179823 for the synthesis of methyl 3-[[(2S)-6-amino-2-[5-[tert-butoxycarbonyl(2-pyridyl)amino]pentanoylamino]hexanoyl]amino]-3-(3,5-dichlorophenyl)propanoate (CAS Nr: 1831059-47-2), followed by saponification of the methyl ester using lithium hydroxide. LC-MS (Method 2): Rt=1.38 min; MS: m/z=608 [M−H]⁻.

### Example S80 Preparation of (3S)-3-[(N-{5-[(tert-butoxycarbonyl)(4-methylpyridin-2-yl)amino]pentanoyl}-3-{[(4-nitrophenoxy)carbonyl]amino}-L-alanyl)amino]-3-(3,5-dichlorophenyl)propanoic acid (Intermediate 80)

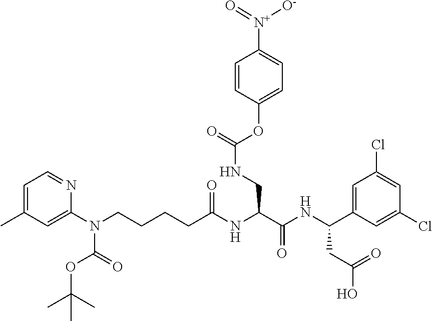 [SMILES: Cc1ccnc(N(CCCCC(=O)N[C@@H](CNC(=O)Oc2ccc([N+](=O)[O-])cc2)C(=O)N[C@@H](CC(=O)O)c2cc(Cl)cc(Cl)c2)C(=O)OC(C)(C)C)c1]

To a solution of (3S)-3-[(3-amino-N-{5-[(tert-butoxycarbonyl)(4-methylpyridin-2-yl)amino]pentanoyl}-L-alanyl)amino]-3-(3,5-dichlorophenyl)propanoic acid (30.0 mg, 49.1 μmol) Intermediate 79 in THF (5.0 ml) was added 4-nitrophenyl carbonochloridate (6.42 mg, 31.8 μmol). The mixture was stirred at rt for 2 h and then concentrated under reduced pressure. The residue was purified over preparative HPLC and freeze dried to afford Intermediate 80 (6.0 mg, 100% purity, 29% yield) as a colorless foam. LC-MS (Method 2): Rₜ=1.77 min; MS: m/z=1056 [M−H].

### Example S81 Preparation of 3-[(N-{(2R,3R)-3-[(2S)-1-{(3R,4S,5S)-4-[(N,N-dimethyl-L-valyl-L-valyl}(methyl)amino]-3-methoxy-5-methylheptanoyl}pyrrolidin-2-yl]-3-methoxy-2-methylpropanoyl}-L-phenylalanyl)amino]propyl N-(3-{2-[2-(2-aminoethoxy)ethoxy]ethoxy}propanoyl)-L-alpha-aspartyl-L-prolyl-L-valinate.trifluoroacetic acid (Intermediate 81)

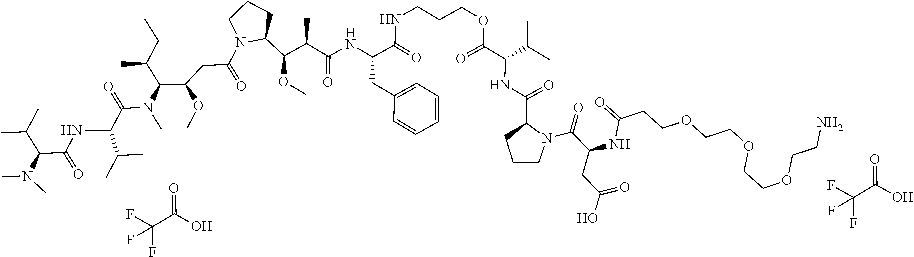 [SMILES: CC[C@H](C)[C@@H]([C@@H](CC(=O)N1CCC[C@H]1[C@H](OC)[C@@H](C)C(=O)N[C@@H](Cc1ccccc1)C(=O)NCCCOC(=O)[C@@H](NC(=O)[C@@H]1CCCN1C(=O)[C@H](CC(=O)O)NC(=O)CCOCCOCCOCCN)C(C)C)OC)N(C)C(=O)[C@@H](NC(=O)[C@H](C(C)C)N(C)C)C(C)C ; O=C(O)C(F)(F)F ; O=C(O)C(F)(F)F]

Step 1: To a solution of trifluoroacetic acid. N,N-dimethyl-L-valyl-N-[(3R,4S,5S)-3-methoxy-1-{(2S)-2-[(1R,2R)-1-methoxy-2-methyl-3-oxo-3-{[(2S)-1-oxo-3-phenyl-1-{[3-(L-valyloxy)propyl]amino}propan-2-yl]amino}propyl]pyrrolidin-1-yl}-5-methyl-1-oxoheptan-4-yl]-N-methyl-L-valinamide (1/1) (25.0 mg, 24.6 μmol) (prepared according to WO2015054659, CASNr=1699749-70-6) and (2S)-1-[(19S)-19-(2-tert-butoxy-2-oxoethyl)-2,2-dimethyl-4,17,20-trioxo-3,8,11,14-tetraoxa-5,18-diazaicosan-20-yl]pyrrolidine-2-carboxylic acid (17.4 mg, 29.5 μmol) Intermediate 3 in DMF (1.0 ml) were added HATU (12.2 mg, 32.0 μmol) and DIEA (13 μl, 74 μmol). The reaction was stirred at RT for 1 h. The residue was purified by prep HPLC and then lyophilised to give tert-butyl (19S)-19-[(2S)-2-{[(3R,4R,7S,15S)-7-benzyl-3-((2S)-1-[(3R,4S,5S)-4-{[(2S)-2-{[(2S)-2-(dimethylamino)-3-methylbutanoyl]amino}-3-methylbutanoyl](methyl)amino}-3-methoxy-5-methylheptanoyl]pyrrolidin-2-yl)-4,16-dimethyl-5,8,14-trioxo-2,13-dioxa-6,9-diazaheptadecan-15-yl]carbamoyl}pyrrolidine-1-carbonyl]-2,2-dimethyl-4,17-dioxo-3,8,11,14-tetraoxa-5,18-diazahenicosan-21-oate (24 mg, 100% purity, 66% yield) as a colorless foam. LC-MS (Method 3): Rt=4.15 min; MS (ESIpos): m/z=1474 [M+H]⁺.

Step 2: To a solution of tert-butyl (19S)-19-[(2S)-2-{[(3R,4R,7S,15S)-7-benzyl-3-{(2S)-1-[(3R,4S,5S)-4-{[(2S)-2-{[(2S)-2-(dimethylamino)-3-methylbutanoyl]amino}-3-methylbutanoyl](methyl)amino}-3-methoxy-5-methylheptanoyl]pyrrolidin-2-yl}-4,16-dimethyl-5,8,14-trioxo-2,13-dioxa-6,9-diazaheptadecan-15-yl]carbamoyl}pyrrolidine-1-carbonyl]-2,2-dimethyl-4,17-dioxo-3,8,11,14-tetraoxa-5,18-diazahenicosan-21-oate (23.0 mg, 15.6 μmol) in DCM (1.0 ml) was added TFA (130 μl). The mixture was stirred at rt for 3 h and then concentrated under reduced pressure. The residue was dissolved in acetonitrile and water and then freeze dried to afford Intermediate 81 (24 mg, 92% purity, 92% yield) as a colorless foam. LC-MS (Method 3): Rₜ=2.81 min; MS: m/z=1318 [M−H]⁻.

### Example S82 Preparation of 3-aminopropyl 4-[(tert-butoxycarbonyl)amino]-2,2-dimethylbutanoate (Intermediate 82)

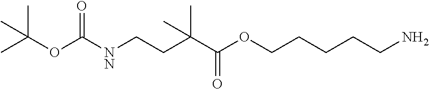

Step 1: To a solution of 4-[(tert-butoxycarbonyl)amino]-2,2-dimethylbutanoic acid (144 mg, 621 μmol) in DMF (1 ml), were added, under Argon, TBTU (230 mg, 717 μmol) and DIEA (330 μl, 1.9 mmol). The mixture was stirred at 50° C. for 3h. A solution of benzyl (3-hydroxypropyl)carbamate (100 mg, 478 μmol) in DMF (0.5 ml) was then added: The mixture was stirred at 50° C. for 20 h. The mixture was then purified over preparative HPLC and freeze dried to afford 3-{[(benzyloxy)carbonyl]amino}propyl 4-[(tert-butoxycarbonyl)amino]-2,2-dimethylbutanoate (124 mg, 99% purity, 61% yield) as a white foam. LC-MS (Method 2): Rₜ=2.09 min; MS (ESIpos): m/z=423 [M+H]⁺.

Step 2: 3-{[(benzyloxy)carbonyl]amino}propyl 4-[(tert-butoxycarbonyl)amino]-2,2-dimethylbutanoate (122 mg, 289 μmol) was dissolved in ethanol (20 ml). Pd/C 10% (12 mg) was added and the reaction was hydrogenated at RT for 4 h and filtered over celite. The mother liquor was concentrated in vacuo to give Intermediate 82 (82 mg, 100% purity, 98% yield).

### Example S83 Preparation of trifluoroacetic acid. N,N-dimethyl-L-valyl-N-[(3R,4S,5S)-1-{(2S)-2-[(3R,4R,7S)-17-amino-7-benzyl-4,15,15-trimethyl-5,8,14-trioxo-2,13-dioxa-6,9-diazaheptadecan-3-yl]pyrrolidin-1-yl}-3-methoxy-5-methyl-1-oxoheptan-4-yl]-N-methyl-L-valinamide (2/1) (Intermediate 83)

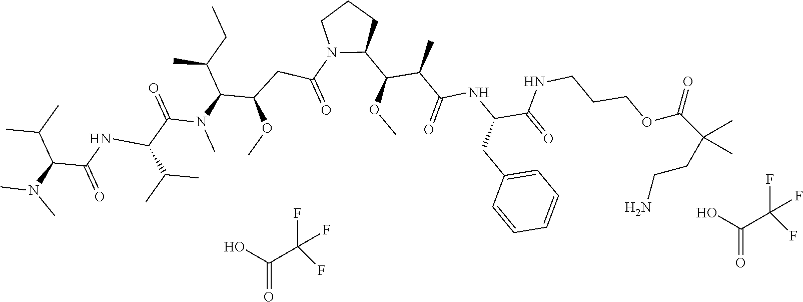

Step 1: To a solution of 3-aminopropyl 4-[(tert-butoxycarbonyl)amino]-2,2-dimethylbutanoate (9.22 mg, 32.0 μmol) Intermediate 82 in DMF (0.5 ml), were added, under Argon, HATU (9.73 mg, 25.6 μmol) and DIEA (6.7 μl, 38 μmol). The mixture was stirred at 50° C. for 3h. A solution of N,N-dimethyl-L-valyl-N-[(3R,4S,5S)-1-{(2S)-2-[(1R,2R)-3-{[(1S)-1-carboxy-2-phenylethyl]amino}-1-methoxy-2-methyl-3-oxopropyl]pyrrolidin-1-yl}-3-methoxy-5-methyl-1-oxoheptan-4-yl]-N-methyl-L-valinamide.trifluoroacetic acid (1/1) (11.0 mg, 12.8 μmol) in DMF (0.05 ml) was then added: The mixture was stirred at 50° C. for 20 h. The mixture was then purified over preparative HPLC and freeze dried to afford 3-{[(benzyloxy)carbonyl]amino}propyl 4-[(tert-butoxycarbonyl)amino]-2,2-dimethylbutanoate (14 mg, 100% purity, quant.) as an amorphous residue. LC-MS (Method 2): Rₜ=1.62 min; MS (ESIpos): m/z=1017 [M+H]⁺.

Step 2: To a solution of N,N-dimethyl-L-valyl-N-[(3R,4S,5S)-1-{(2S)-2-[(3R,4R,7S)-7-benzyl-4,15,15,21,21-pentamethyl-5,8,14,19-tetraoxo-2,13,20-trioxa-6,9,18-triazadocosan-3-yl]pyrrolidin-1-yl}-3-methoxy-5-methyl-1-oxoheptan-4-yl]-N-methyl-L-valinamide (38.0 mg, 37.4 μmol) in DCM (4 ml) was added TFA (1 ml). The mixture was stirred 2 h at rt and then concentrated under reduced pressure. The residue was dissolved in acetonitrile and water and then freeze dried to afford Intermediate 83 (40 mg, 100% purity, 93% yield) as a white foam. LC-MS (Method 2): Rₜ=1.04 min; MS (ESIneg): m/z=961 [M−H+HCOO]⁻.

### Example S84 Preparation of trifluoroacetic acid. N,N-dimethyl-L-valyl-N-[(3R,4S,5S)-1-{(2S)-2-[(3R,4R,7S)-7-benzyl-4,15,15-trimethyl-5,8,14-trioxo-17-(L-valylamino)-2,13-dioxa-6,9-diazaheptadecan-3-yl]pyrrolidin-1-yl}-3-methoxy-5-methyl-1-oxoheptan-4-yl]-N-methyl-L-valinamide (1/1) (Intermediate 84)

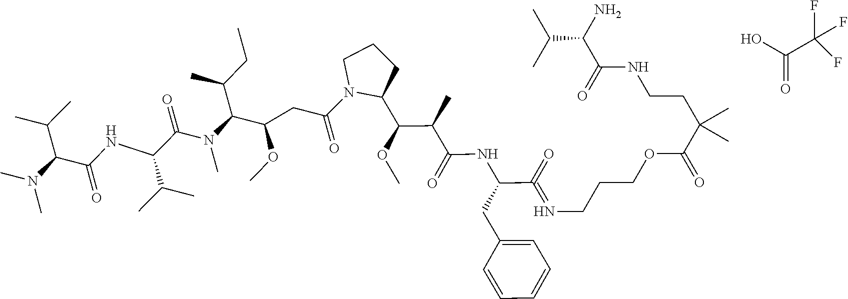

Step 1: To a solution of trifluoroacetic acid. N,N-dimethyl-L-valyl-N-[(3R,4S,5S)-1-{(2S)-2-[(3R,4R,7S)-17-amino-7-benzyl-4,15,15-trimethyl-5,8,14-trioxo-2,13-dioxa-6,9-diazaheptadecan-3-yl]pyrrolidin-1-yl}-3-methoxy-5-methyl-1-oxoheptan-4-yl]-N-methyl-L-valinamide (2/1) (39.0 mg, 34.1 μmol) (Intermediate 83) in DCM (1.0 ml), were added 2,5-dioxopyrrolidin-1-yl N-(tert-butoxycarbonyl)-L-valinate (21.4 mg, 68.2 μmol), 4-(dimethylamino)pyridin (4.16 mg, 34.1 μmol) and DIEA (12 μl, 68 μmol). The mixture was stirred 2 h at rt and then concentrated under reduced pressure. The residue was purified over preparative HPLC and freeze dried to afford trifluoroacetic acid. N,N-dimethyl-L-valyl-N-[(3R,4S,5S)-1-{(2S)-2-[(3R,4R,7S)-7-benzyl-17-{[N-(tert-butoxycarbonyl)-L-valyl]amino}-4,15,15-trimethyl-5,8,14-trioxo-2,13-dioxa-6,9-diazaheptadecan-3-yl]pyrrolidin-1-yl}-3-methoxy-5-methyl-1-oxoheptan-4-yl]-N-methyl-L-valinamide (1/1) (38 mg, 100% purity, 91% yield) as a colorless foam. LC-MS (Method 2): R: =1.66 min; MS (ESIpos): m/z=1116 [M+H]⁺.

Step 2: To a solution of trifluoroacetic acid. N,N-dimethyl-L-valyl-N-[(3R,4S,5S)-1-{(2S)-2-[(3R,4R,7S)-7-benzyl-17-{[N-(tert-butoxycarbonyl)-L-valyl]amino}-4,15,15-trimethyl-5,8,14-trioxo-2,13-dioxa-6,9-diazaheptadecan-3-yl]pyrrolidin-1-yl}-3-methoxy-5-methyl-1-oxoheptan-4-yl]-N-methyl-L-valinamide (1/1) (37.0 mg, 30.1 μmol) in DCM (2 ml) was added TFA (500 μl). The mixture was stirred 2 h at rt and then concentrated under reduced pressure. The residue was dissolved in acetonitrile and water and freeze dried to afford Intermediate 84 (33 mg, 90% purity, 87% yield). LC-MS (Method 2): Rₜ=1.13 min; MS (ESIneg): m/z=1060 [M−H+CH₃COOH]⁻.

### Example S85 Preparation of N-(3-{2-[2-(2-aminoethoxy)ethoxy]ethoxy}propanoyl)-L-alpha-aspartyl-L-prolyl-N-(4-{3-[(N-{(2R,3R)-3-[(2S)-1-{(3R,4S,5S)-4-[(N,N-dimethyl-L-valyl-L-valyl}(methyl)amino]-3-methoxy-5-methylheptanoyl}pyrrolidin-2-yl]-3-methoxy-2-methylpropanoyl}-L-phenylalanyl)amino]propoxy)-3,3-dimethyl-4-oxobutyl)-L-valinamide.trifluoroacetic acid (1/1) (Intermediate 85)

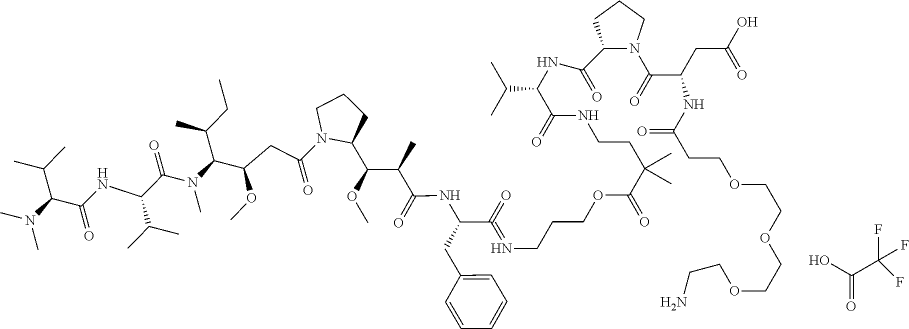

Step 1: To a solution of trifluoroacetic acid. N,N-dimethyl-L-valyl-N-[(3R,4S,5S)-1-{(2S)-2-[(3R,4R,7S)-7-benzyl-,15,15-trimethyl-5,8,14-trioxo-17-(L-valylamino)-2,13-dioxa-6,9-diazaheptadecan-3-yl]pyrrolidin-1-yl}-3-methoxy-5-methyl-1-oxoheptan-4-yl]-N-methyl-L-valinamide (1/1) (33.0 mg, 90% purity, 26.3 μmol) Intermediate 84 in DMF (1.3 nil) were added HATU (13.0 mg, 34.2 μmol) and DIEA (14 μl, 79 μmol). (2S)-1-[(19S)-19-(2-tert-butoxy-2-oxoethyl)-2,2-dimethyl-4,17,20-trioxo-3,8,11,14-tetraoxa-5,18-diazaicosan-20-yl]pyrrolidine-2-carboxylic acid (18.6 mg, 31.6 μmol) Intermediate 3 was then added and the reaction was stirred at RT for 1 h. The residue was purified by prep HPLC and then lyophilised to give trifluoroacetic acid. tert-butyl (19S)-19-[(2S)-2-{[(3R,4R,7S,20S)-7-benzyl-3-{(2S)-1-[(3R,4S,5S)-4-{[(2S)-2-{[(2S)-2-(dimethylamino)-3-methylbutanoyl]amino}-3-methylbutanoyl](methyl)amino}-3-methoxy-5-methylheptanoyl]pyrrolidin-2-yl}-4,15,15,21-tetramethyl-5,8,14,19-tetraoxo-2,13-dioxa-6,9,18-triazadocosan-20-yl]carbamoyl}pyrrolidine-1-carbonyl]-2,2-dimethyl-4,17-dioxo-3,8,11,14-tetraoxa-5,18-diazahenicosan-21-oate (1/1) (44 mg, 70% purity, 69% yield) as an amorphous residue. LC-MS (Method 3): Rt=4.23 min; MS (ESIpos): mi/z=1587 [M+H]⁺.

Step 2: To a solution of trifluoroacetic acid. tert-butyl (19S)-19-[(2S)-2-{[(3R,4R,7S,20S)-7-benzyl-3-{(2S)-1-[(3R,4S,5S)-4-{[(2S)-2-{[(2S)-2-(dimethylamino)-3-methylbutanoyl]amino}-3-methylbutanoyl](methyl)amino}-3-methoxy-5-methylheptanoyl]pyrrolidin-2-yl}-4,15,15,21-tetramethyl-5,8,14,19-tetraoxo-2,13-dioxa-6,9,18-triazadocosan-20-yl]carbamoyl}pyrrolidine-1-carbonyl]-2,2-dimethyl-4,17-dioxo-3,8,11,14-tetraoxa-5,18-diazahenicosan-21-oate (1/1) (43.0 mg, 70% purity, 17.7 μmol) in DCM (1.5 ml) was added TFA (500 μl). The mixture was stirred at rt for 2 h and then concentrated under reduced pressure. The residue was dissolved in acetonitrile and water and then freeze dried to afford Intermediate 85 (25 mg, 94% purity, 86% yield) as a white foam. LC-MS (Method 3): R:=2.95 min, MS: m/z=716 [M+2H]²⁺.

### Example S86 Preparation of 4-amino-2-methylbutan-2-yl N-(tert-butoxycarbonyl)-L-valinate (Intermediate 86)

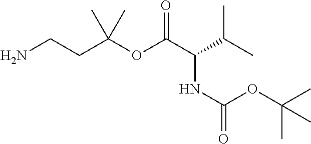 [SMILES: CC(C)[C@H](NC(=O)OC(C)(C)C)C(=O)OC(C)(C)CCN]

Step 1: To a solution of benzyl (3-hydroxy-3-methylbutyl)carbamate (50.0 mg, 211 μmol) in DCM (1 ml), were added, at 0° C., DMAP (25.7 mg, 211 μmol) and N-(tert-butoxycarbonyl)-L-valine (91.6 mg, 421 μmol). The mixture was stirred at 0° C. for 10 min and 1-(3-dimethylaminopropyl)-3-ethylcarbodiimidhydrochlorid (80.8 mg, 421 μmol) was added. The reaction was then stirred overnight at rt. The mixture was purified over preparative HPLC and freeze dried to afford 3 4-{[(benzyloxy)carbonyl]amino}-2-methylbutan-2-yl N-(tert-butoxycarbonyl)-L-valinate (36 mg, 91% purity, 36% yield) as a colorless oil. LC-MS (Method 4): Rₜ=2.33 min; MS (ESIpos): m/z=459 [M+Na]+.

Step 2: 4-{[(benzyloxy)carbonyl]amino}-2-methylbutan-2-yl N-(tert-butoxycarbonyl)-L-valinate (97.0 mg, 85% purity, 189 μmol) was dissolved in ethanol (20 ml). Pd/C 10% (12 mg) was added and the reaction was hydrogenated at RT for 4h. Pd/C 10% (10 mg) was then added and the reaction was hydrogenated at RT overnight and filtered over celite. The mother liquor was concentrated in vacuo to give Intermediate 86 (76 mg, 75% purity, 100% yield).

### Example S87 Preparation of trifluoroacetic acid. N,N-dimethyl-L-valyl-N-[(3R,4S,5S)-3-methoxy-1-{(2S)-2-[(1R,2R)-1-methoxy-2-methyl-3-{[(2S)-1-{[3-methyl-3-(L-valyloxy)butyl]amino}-1-oxo-3-phenylpropan-2-yl]amino}-3-oxopropyl]pyrrolidin-1-yl}-5-methyl-1-oxoheptan-4-yl]-N-methyl-L-valinamide (1/1) (Intermediate 87)

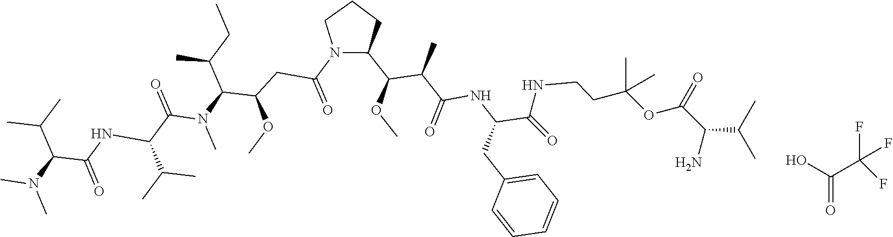

Step 1: To a solution of 4-amino-2-methylbutan-2-yl N-(tert-butoxycarbonyl)-L-valinate (29.3 mg, 75% purity, 72.7 μmol) Intermediate 86 in DMF (0.9 ml), were added HATU (22.1 mg, 58.1 μmol) and DIEA (15 μl, 87 μmol). The mixture was stirred at 50° C. for 1h. A solution of N N,N-dimethyl-L-valyl-N-[(3R,4S,5S)-1-{(2S)-2-[(1R,2R)-3-{[(1S)-1-carboxy-2-phenylethyl]amino}-1-methoxy-2-methyl-3-oxopropyl]pyrrolidin-1-yl}-3-methoxy-5-methyl-1-oxoheptan-4-yl]-N-methyl-L-valinamide.trifluoroacetic acid (1/1) (25.0 mg, 29.1 μmol) in DMF (0.05 ml) was then added: The mixture was stirred at 50° C. for 2 h. The mixture was then purified over preparative HPLC and freeze dried to afford N,N-dimethyl-L-valyl-N-[(3R,4S,5S)-1-{(2S)-2-[(1R,2R)-3-{[(1S)-1-carboxy-2-phenylethyl]amino}-1-methoxy-2-methyl-3-oxopropyl]pyrrolidin-1-yl}-3-methoxy-5-methyl-1-oxoheptan-4-yl]-N-methyl-L-valinamide.trifluoroacetic acid (1/1) (18 mg, 81% purity, 49% yield) as an amorphous residue. LC-MS (Method 2): Rₜ=1.74 min; MS (ESIpos): m/z=1031 [M+H]⁺.

Step 2: To a solution of N,N-dimethyl-L-valyl-N-[(3R,4S,5S)-1-{(2S)-2-[(1R,2R)-3-(((2S)-1-[(3-{[N-(tert-butoxycarbonyl)-L-valyl]oxy)-3-methylbutyl)amino]-1-oxo-3-phenylpropan-2-yl}amino)-1-methoxy-2-methyl-3-oxopropyl]pyrrolidin-1-yl}-3-methoxy-5-methyl-1-oxoheptan-4-yl]-N-methyl-L-valinamide (18.0 mg, 81% purity, 14.2 μmol) in DCM (1.5 ml) was added TFA (0.38 ml). The mixture was stirred 2 h at rt and then concentrated under reduced pressure. The residue was dissolved in acetonitrile and water and then freeze dried to afford Intermediate 87 (14.5 mg, 95% purity, 93% yield). LC-MS (Method 2): Rₜ=1.08 min; MS (ESIneg): m/z=975 [M−H+HCOO—H]⁻.

### Example S88 Preparation of (3R)-3-[({4-[({(14S)-14-[(2S)-2-{[(3R,4R,7S,15S)-7-benzyl-3-{(2S)-1-[(3R,4S,5S)-4-{[(2S)-2-{[(2S)-2-(dimethylamino)-3-methylbutanoyl]amino}-3-methylbutanoyl](methyl)amino}-3-methoxy-5-methylheptanoyl]pyrrolidin-2-yl}-4,12,12,16-tetramethyl-5,8,14-trioxo-2,13-dioxa-6,9-diazaheptadecan-15-yl]carbamoyl}pyrrolidine-1-carbonyl]-18,18-dimethyl-12,16-dioxo-3,6,9,17-tetraoxa-13-azanonadecan-1-yl}carbamoyl)amino]phenyl}carbamoyl)amino]-3-[3-({3-[(propylcarbamoyl)amino]benzene-1-sulfonyl}amino)phenyl]propanoic acid (Intermediate 88)

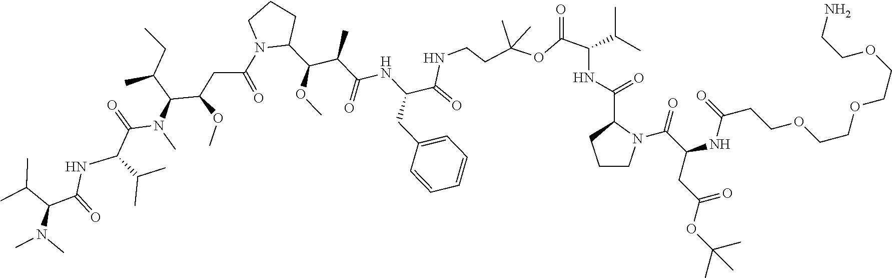 [SMILES: CC[C@H](C)[C@@H]([C@@H](CC(=O)N1CCCC1[C@H](OC)[C@@H](C)C(=O)N[C@@H](Cc1ccccc1)C(=O)NCCC(C)(C)OC(=O)[C@@H](NC(=O)[C@@H]1CCCN1C(=O)[C@H](CC(=O)OC(C)(C)C)NC(=O)CCOCCOCCOCCN)C(C)C)OC)N(C)C(=O)[C@@H](NC(=O)[C@H](C(C)C)N(C)C)C(C)C]

Step 1: To a solution of trifluoroacetic acid. N,N-dimethyl-L-valyl-N-[(3R,4S,5S)-3-methoxy-1-{(2S)-2-[(1R,2R)-1-methoxy-2-methyl-3-{[(2S)-1-{[3-methyl-3-(L-valyloxy)butyl]amino}-1-oxo-3-phenylpropan-2-yl]amino}-3-oxopropyl]pyrrolidin-1-yl}-5-methyl-1-oxoheptan-4-yl]-N-methyl-L-valinamide (1/1) (14.5 mg, 95% purity, 13.1 μmol) Intermediate 87 in DMF (0.8 ml) were added HATU (6.49 mg, 17.1 μmol) and DIEA (6.9 μl, 39 μmol). (2S)-1-[(19S)-19-(2-tert-butoxy-2-oxoethyl)-2,2-dimethyl-4,17,20-trioxo-3,8,11,14-tetraoxa-5,18-diazaicosan-20-yl]pyrrolidine-2-carboxylic acid (9.28 mg, 15.7 μmol) Intermediate 3 was then added and the reaction was stirred at RT for 2 h. The residue was purified by prep HPLC and then lyophilised to give tert-butyl (19S)-19-[(2S)-2-{[(3R,4R,7S,15S)-7-benzyl-3-{(2S)-1-[(3R,4S,5S)-4-{[(2S)-2-{[(2S)-2-(dimethylamino)-3-methylbutanoyl]amino}-3-methylbutanoyl](methyl)amino}-3-methoxy-5-methylheptanoyl]pyrrolidin-2-yl}-4,12,12,16-tetramethyl-5,8,14-trioxo-2,13-dioxa-6,9-diazaheptadecan-15-yl]carbamoyl}pyrrolidine-1-carbonyl]-2,2-dimethyl-4,17-dioxo-3,8,11,14-tetraoxa-5,18-diazahenicosan-21-oate (16 mg, 100% purity, 81% yield) as an amorphous residue. LC-MS (Method 3): Rt=4.29 min; MS (ESIpos): m/z=751 [M+2H]²⁺.

Step 2: To a solution of tert-butyl (19S)-19-[(2S)-2-{[(3R,4R,7S,15S)-7-benzyl-3-{(2S)-1-[(3R,4S,5S)-4-{[(2S)-2-{[(2S)-2-(dimethylamino)-3-methylbutanoyl]amino}-3-methylbutanoyl](methyl)amino}-3-methoxy-5-methylheptanoyl]pyrrolidin-2-yl}-4,12,12,16-tetramethyl-5,8,14-trioxo-2,13-dioxa-6,9-diazaheptadecan-15-yl]carbamoyl}pyrrolidine-1-carbonyl]-2,2-dimethyl-4,17-dioxo-3,8,11,14-tetraoxa-5,18-diazahenicosan-21-oate (16.0 mg, 10.7 μmol) in DCM (1.0 ml) was added TFA (280 μl). The mixture was stirred at rt for 2 h and then concentrated under reduced pressure. The residue was dissolved in acetonitrile and water and then freeze dried to afford Intermediate 88 (14 mg, 83% purity, 77% yield) as a white foam. LC-MS (Method 3): Rₜ=3.26 min; MS: m/z=701 [M+2H]²⁺.

### Compounds

### Example 1: Preparation of tetrasodium; (3S)-3-[3-[2-[2-[2-[[(4S)-4-[[(2S)-2,6-bis[3-[2-[2-[2-[[4-[[(1R)-2-carboxylato-1-[3-[[3-(propylcarbamoylamino)phenyl]sulfonylamino]phenyl]ethyl]carbamoylamino]phenyl]carbamnoylamino]ethoxy]ethoxy]ethoxy]propanoylamino]hexanoyl]amino]-5-[2-[2-[2-[3-[[(1S)-1-(carboxylatomethyl)-2-[(2S)-2-[[(1 S)-1-[[(19S)-10,19-diethyl-14,18-dioxo-17-oxa-3,13-diazapentacyclo[11.8.0.0\hat{ }(2,11).0\hat{ }(4,9).0\hat{ }(15,20)]henicosa-1(21),2,4(9),5,7,10,15(20)-heptaen-19-yl]oxycarbonyl]-2-methyl-propyl]carbamoyl]pyrrolidin-1-yl]-2-oxo-ethyl]amino]-3-oxo-propoxy]ethoxy]ethoxy]ethylamino]-5-oxo-pentanoyl]amino]ethoxy]ethoxy]ethoxy]propanoylamino]-4-[(2S)-2-[[(1 S)-1-[[(19S)-10,19-diethyl-14,18-dioxo-17-oxa-3,13-diazapentacyclo[11.8.0.0\hat{ }{2,11}.0\hat{ }{4,9}.0\hat{ }{15,20}]henicosa-1(21),2,4(9),5,7,10,15(20)-heptaen-19-yl]oxycarbonyl]-2-methyl-propyl]carbamoyl]pyrrolidin-1-yl]-4-oxo-butanoate (Compound 1)

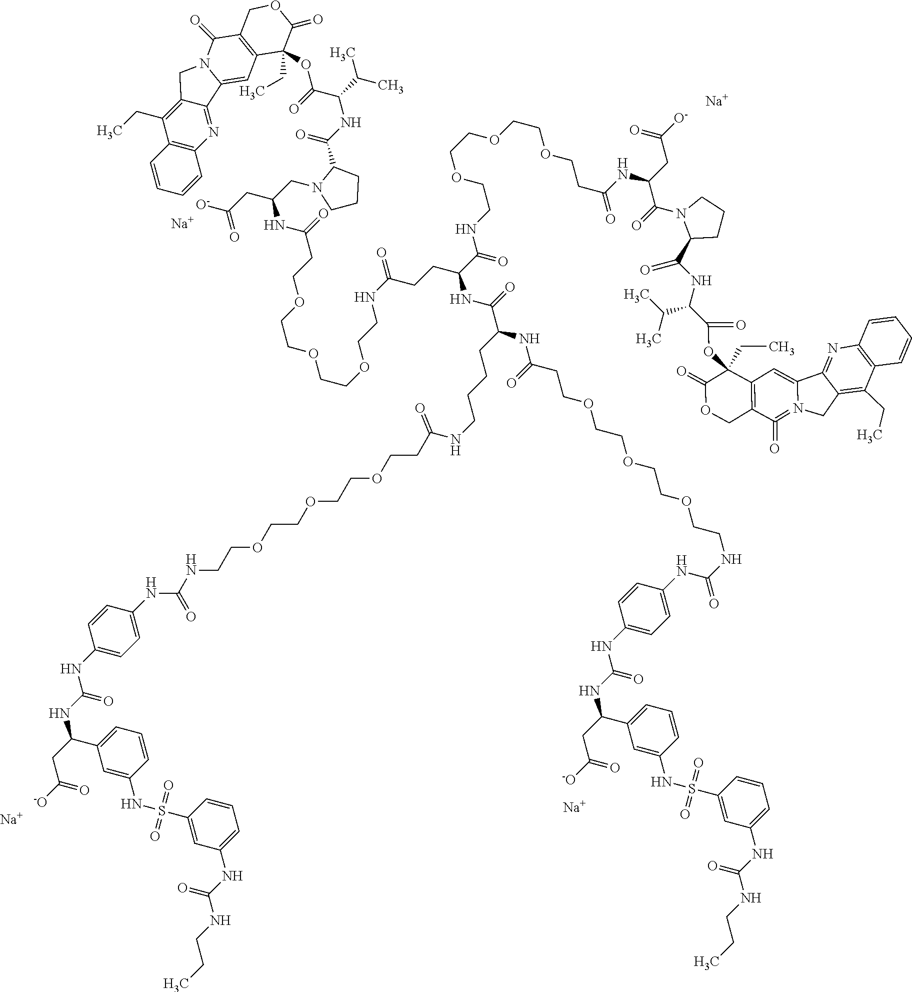

Step 1: To a solution of (3S,19S,37S)-19-{[(2S)-19-amino-2-(3-{2-[2-(2-aminoethoxy)ethoxy]ethoxy}propanamido)-8-oxo-11,14,17-trioxa-7-azanonadecanan-1-oyl]amino}-3,37-bis[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-5,18,22,35-tetraoxo-8,11,14,26,29,32-hexaoxa-4,17,23,36-tetraazanonatriacontane-1,39-dioic acid-trifluoroacetic acid (1/2) (20.0 mg, 7.53 μmol) (Intermediate 14) in DMF (8 ml), were added (3R)-3-{[(4-{[(4-nitrophenoxy)carbonyl]amino}phenyl)carbamoyl]amino}-3-[3-({3-[(propylcarbamoyl)amino]benzene-1-sulfonyl}amino)phenyl]propanoic acid (12.5 mg, 95% purity, 16.6 μmol) (Intermediate 2) and DIEA (6.6 μl, 38 μmol). The mixture was stirred at rt for 1 h 30 and then concentrated under reduced pressure. The residue was purified over preparative HPLC and freeze dried to afford (3S,19S,37S)-19-({(2S)-21-{4-[({(1R)-2-carboxy-1-[3-({3-[(propylcarbamoyl)amino]benzene-1-sulfonyl}amino)phenyl]ethyl}carbamoyl}amino]anilino}-2-[(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)amino]-8,21-dioxo-11,14,17-trioxa-7,20-diazahenicosanan-1-oyl)amino)-3,37-bis[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-5,18,22,35-tetraoxo-8,11,14,26,29,32-hexaoxa-4,17,23,36-tetraazanonatriacontane-1,39-dioic acid (16 mg, 100% purity, 59% yield). LC-MS (Method 3): Rₜ=4.58 min; MS (ESIpos): m/z=1795 [M+2H]²⁺.

Step 2: To a solution of (3S,19S,37S)-19-({(2S)-21-{4-[({(1R)-2-carboxy-1-[3-({3-[(propylcarbamoyl)amino]benzene-1-sulfonyl}amino)phenyl]ethyl}carbamoyl)amino]anilino}-2-[(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)amino]-8,21-dioxo-11,14,17-trioxa-7,20-diazahenicosanan-1-oyl}amino)-3,37-bis[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-5,18,22,35-tetraoxo-8,11,14,26,29,32-hexaoxa-4,17,23,36-tetraazanonatriacontane-1,39-dioic acid (16.0 mg, 4.46 μmol) in dioxane/water (1:1, 8 mL) was added a sodium hydroxide solution (18 μl, 1 M, 18 μmol). The solution was freeze-dried to give Compound 1 (16.0 mg, 100% purity, 98% yield). LC-MS: R: =4.59 min; MS (ESIpos): m/z=1795 [M+2H]²⁺. (Method 3): ¹H-NMR (600 MHz, DMSO-d6) δ[ppm]: 0.068 (1.61), 0.808 (4.01), 0.826 (8.62), 0.844 (4.76), 0.883 (2.81), 0.902 (5.70), 0.922 (8.64), 0.939 (6.79), 1.221 (1.68), 1.237 (2.81), 1.282 (2.77), 1.302 (5.24), 1.321 (3.08), 1.342 (2.22), 1.360 (3.40), 1.378 (3.15), 1.397 (1.66), 1.920 (1.49), 2.071 (1.22), 2.189 (1.89), 2.206 (2.16), 2.287 (2.08), 2.303 (1.51), 2.328 (2.79), 2.367 (1.85), 2.587 (1.24), 2.671 (1.45), 2.711 (1.28), 2.738 (0.75), 2.933 (2.29), 2.947 (2.33), 3.213 (4.91), 3.419 (7.88), 3.442 (16.00), 3.450 (15.54), 3.476 (7.49), 3.508 (15.79), 3.568 (8.24), 3.585 (4.07), 3.790 (1.76), 3.997 (1.15), 4.890 (1.13), 4.908 (1.17), 4.960 (1.45), 5.331 (2.66), 5.479 (4.63), 6.182 (0.57), 6.698 (1.24), 6.936 (1.36), 6.954 (1.74), 7.053 (1.47), 7.072 (2.14), 7.093 (1.07), 7.185 (2.39), 7.207 (4.38), 7.235 (4.76), 7.258 (2.45), 7.268 (2.33), 7.288 (2.54), 7.308 (1.47), 7.342 (1.66), 7.603 (1.22), 7.719 (1.78), 7.738 (1.76), 7.854 (2.18), 8.026 (2.18), 8.047 (2.33), 8.262 (1.91), 8.282 (2.14), 8.426 (0.92), 8.761 (1.17), 9.945 (0.65).

### Example 2: Preparation of trisodium (3S)-3-[3-[2-[2-[2-[[5-[[(5S)-5-[[5-[2-[2-[2-[3-[[(1S)-1-(carboxylatomethyl)-2-[(2S)-2-[[(1S)-1-[[(19S)-10,19-diethyl-14,18-dioxo-17-oxa-3,13-diazapentacyclo[11.8.0.0^{2,11}0.^{4,9}.0^{15,20},]henicosa-1(21),2,4(9),5,7,10,15(20)-heptaen-19-yl]oxycarbonyl]-2-methyl-propyl]carbamoyl]pyrrolidin-1-yl]-2-oxo-ethyl]amino]-3-oxo-propoxy]ethoxy]ethoxy]ethylamino]-5-oxo-pentanoyl]amino]-6-[2-[2-[2-[2-[[4-[[(1R)-2-carboxylato-1-[3-[[3-(propylcarbamoylamino)phenyl]sulfonylamino]phenyl]ethyl]carbamoylamino]phenyl]carbamoylamino]ethoxy]ethoxy]ethoxy]ethylamino]-6-oxo-hexyl]amino]-5-oxo-pentanoyl]amino]ethoxy]ethoxy]ethoxy]propanoylamino]-4-[(2S)-2-[[(1S)-1-[[(19S)-10,19-diethyl-14,18-dioxo-17-oxa-3,13-diazapentacyclo[11.8.0.0^{2,11}.0^{4,9}.0^{15,20}]henicosa-1(21),2,4(9),5,7,10,15(20)-heptaen-19-yl]oxycarbonyl]-2-methyl-propyl]carbamoyl]pyrrolidin-1-yl]-4-oxo-butanoate (Compound 2)

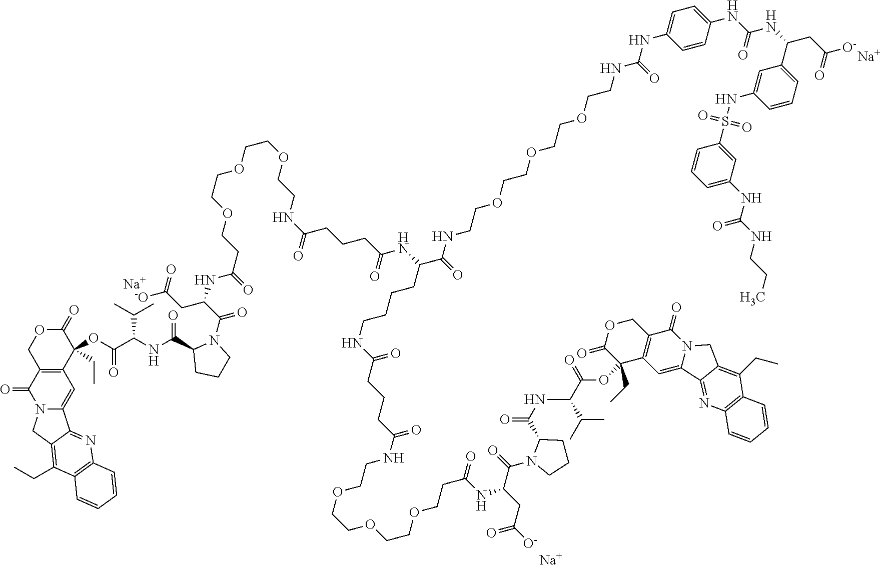

Step 1: To a solution of (3S,24S,49S)-24-[(2-{2-[2-(2-aminoethoxy)ethoxy]ethoxy}ethyl)carbamoyl]-3,49-bis[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-5,18,22,30,34,47-hexaoxo-8,11,14,38,41,44-hexaoxa-4,17,23,29,35,48-hexaazahenpentacontane-1,51-dioic acid. trifluoroacetic acid (1/1) (28.0 mg, 92% purity, 10.7 μmol) (Intermediate 16) in DMF (8 ml), were added (3R)-3-{[(4-{[(4-nitrophenoxy)carbonyl]amino}phenyl)carbamoyl]amino}-3-[3-({3-[(propylcarbamoyl)amino]benzene-1-sulfonyl}amino)phenyl]propanoic acid (8.50 mg, 11.8 μmol) (Intermediate 2) and DIEA (7.5 μl, 43 μmol). The mixture was stirred at rt for 15 min and then concentrated under reduced pressure. The residue was purified over preparative HPLC and freeze dried to afford (3S,24S,49S)-24-[(13-{4-[({(1R)-2-carboxy-1-[3-({3-[(propylcarbamoyl)amino]benzene-1-sulfonyl}amino)phenyl]ethyl}carbamoyl)amino]anilino}-13-oxo-3,6,9-trioxa-12-azatridecane-1-yl)carbamoyl]-3,49-bis[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-5,18,22,30,34,47-hexaoxo-8,11,14,38,41,44-hexaoxa-4,17,23,29,35,48-hexaazahenpentacontane-1,51-dioic acid (16 mg, 100% purity, 52% yield). LC-MS (Method 3): Rₜ=4.38 min; MS (ESIpos): m/z=1438 [M+2H]²⁺

Step 2: To a solution of (3S,24S,49S)-24-[(13-{4-[({(1R)-2-carboxy-1-[3-({3-[(propylcarbamoyl)amino]benzene-1-sulfonyl}amino)phenyl]ethyl}carbamoyl)amino]anilino}-13-oxo-3,6,9-trioxa-12-azatridecane-1-yl)carbamoyl]-3,49-bis[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-5,18,22,30,34,47-hexaoxo-8,11,14,38,41,44-hexaoxa-4,17,23,29,35,48-hexaazahenpentacontane-1,51-dioic acid (16.0 mg, 5.56 μmol) in dioxane/water (1:1, 6 mL) was added a sodium hydroxide solution (17 μl, 1 M, 17 μmol). The solution was freeze-dried to give Compound 2 (16.0 mg, 100% purity, 98% yield). LC-MS (Method 3): Rₜ=4.38 min; MS (ESIpos): m/z=1438 [M+2H]²⁺. ¹H-NMR (600 MHz, DMSO-d6) δ [ppm]: −0.007 (10.69), 0.062 (1.35), 0.800 (1.68), 0.818 (3.63), 0.837 (2.08), 0.875 (6.20), 0.899 (5.03), 0.915 (4.01), 1.005 (0.46), 1.229 (1.22), 1.280 (2.26), 1.299 (4.75), 1.317 (2.92), 1.346 (1.65), 1.363 (1.45), 1.382 (0.69), 1.677 (1.32), 1.846 (0.61), 2.010 (2.67), 2.028 (2.62), 2.078 (1.35), 2.097 (1.57), 2.217 (1.83), 2.288 (1.19), 2.322 (1.50), 2.341 (1.42), 2.361 (1.70), 2.664 (1.04), 2.705 (0.84), 2.910 (1.09), 2.925 (1.17), 2.956 (1.14), 2.971 (1.07), 3.151 (2.92), 3.195 (3.15), 3.424 (15.26), 3.429 (16.00), 3.487 (4.04), 3.505 (6.91), 3.545 (1.63), 3.561 (1.65), 3.574 (1.40), 3.830 (0.86), 3.960 (0.56), 4.147 (0.48), 4.815 (0.74), 4.958 (0.46), 5.176 (0.69), 5.317 (2.74), 5.451 (4.06), 6.626 (0.58), 6.642 (0.69), 6.925 (0.56), 6.946 (0.79), 7.027 (0.63), 7.046 (0.94), 7.157 (0.69), 7.177 (0.84), 7.227 (4.17), 7.265 (0.71), 7.285 (0.94), 7.305 (0.51), 7.388 (0.51), 7.493 (1.09), 7.586 (0.91), 7.687 (0.69), 7.705 (1.30), 7.723 (0.91), 7.831 (1.14), 7.852 (1.70), 7.934 (0.97), 7.993 (1.96), 8.013 (1.55), 8.174 (0.97), 8.196 (0.71), 8.253 (1.42), 8.275 (1.32), 8.771 (0.61), 8.873 (0.94), 11.250 (0.63).

### Example 3 Preparation of trisodium (3S,19S,37S)-19-[(14-{4-[({(1R)-2-carboxylato-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)amino]-3,37-bis[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-5,18,22,35-tetraoxo-8,11,14,26,29,32-hexaoxa-4,17,23,36-tetraazanonatriacontane-1,39-dioate (Compound 3)

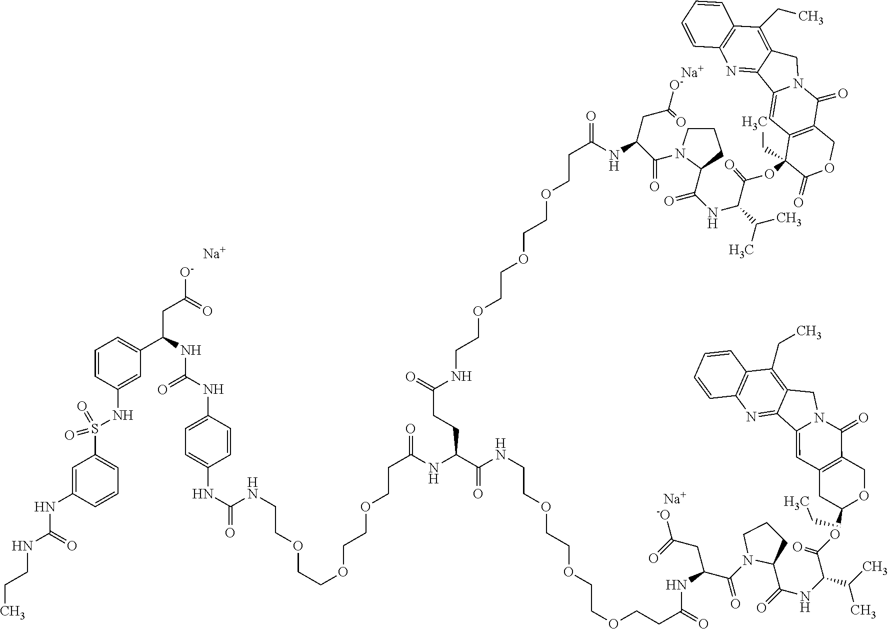

Step 1: To a solution of (3S,19S,37S)-19-(3-{2-[2-(2-aminoethoxy)ethoxy]ethoxy}propanamido)-3,37-bis[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-5,18,22,35-tetraoxo-8,11,14,26,29,32-hexaoxa-4,17,23,36-tetraazanonatriacontane-1,39-dioic acid-trifluoroacetic acid (1/1) (12.0 mg, 5.43 μmol) (Intermediate 18) in DMF (5 ml), were added (3R)-3-{[(4-{[(4-nitrophenoxy)carbonyl]amino}phenyl)carbamoyl]amino}-3-[3-({3-[(propylcarbamoyl)amino]benzene-1-sulfonyl}amino)phenyl]propanoic acid (4.52 mg, 95% purity, 5.97 μmol) (Intermediate 2) and DIEA (2.4 μl, 14 μmol). The mixture was stirred at rt for 15 min and then concentrated under reduced pressure. The residue was purified over preparative HPLC and dried under vacuum to afford (3S,19S,37S)-19-[(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)amino]-3,37-bis[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-5,18,22,35-tetraoxo (7.9 mg, 100% purity, 54% yield). LC-MS (Method 3): Rₜ=4.46 min; MS (ESIpos): m/z=1338 [M+2H]²⁺.

Step 2: To a solution of (3S,19S,37S)-19-[(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)amino]-3,37-bis[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-5,18,22,35-tetraoxo-8,11,14,26,29,32-hexaoxa-4,17,23,36-tetraazanonatriacontane-1,39-dioic acid (7.80 mg, 100% purity, 2.91 μmol) in dioxane/water (1:1, 3 mL) was added a sodium hydroxide solution (8.7 μl, 1 M, 8.7 μmol). The solution was freeze-dried to give Compound 3 (7.9 mg, 100% purity, 99% yield) as a colorless foam. LC-MS (Method 3): Rₜ=4.47 min; MS (ESIpos): m/z=1339 [M+2H]²⁺. ¹H-NMR (600 MHz, DMSO-d6) δ[ppm]: 0.812 (2.53), 0.825 (4.94), 0.837 (2.79), 0.863 (5.75), 0.871 (6.39), 0.876 (7.61), 0.889 (3.86), 0.902 (4.70), 0.912 (4.62), 1.233 (0.84), 1.290 (3.11), 1.303 (6.10), 1.315 (3.25), 1.342 (1.02), 1.353 (1.68), 1.365 (1.66), 1.378 (0.87), 1.685 (0.46), 1.827 (0.99), 1.984 (1.07), 2.072 (1.36), 2.124 (0.90), 2.148 (0.96), 2.234 (1.89), 2.245 (2.56), 2.257 (2.15), 2.268 (1.57), 2.350 (1.31), 2.384 (2.96), 2.423 (1.31), 2.564 (0.75), 2.612 (1.13), 2.652 (1.05), 2.665 (0.75), 2.691 (0.61), 2.915 (1.28), 2.927 (1.31), 3.129 (1.45), 3.140 (1.92), 3.148 (1.89), 3.159 (2.26), 3.172 (2.50), 3.184 (2.21), 3.194 (2.21), 3.207 (1.89), 3.388 (4.27), 3.398 (3.02), 3.409 (3.89), 3.424 (16.00), 3.431 (9.47), 3.476 (3.48), 3.489 (3.80), 3.501 (7.14), 3.519 (2.26), 3.524 (2.58), 3.530 (5.17), 3.545 (1.92), 3.566 (5.43), 3.584 (3.02), 3.588 (2.90), 3.597 (2.61), 3.609 (1.48), 3.625 (0.67), 3.736 (0.84), 3.749 (1.31), 3.764 (0.81), 3.830 (1.10), 3.838 (0.93), 3.852 (0.87), 3.866 (0.84), 3.990 (0.55), 4.023 (0.96), 4.186 (0.70), 4.328 (1.42), 4.415 (0.44), 4.423 (0.44), 4.432 (0.44), 4.649 (0.52), 4.800 (0.87), 4.837 (0.90), 4.958 (0.61), 5.226 (1.05), 5.240 (1.05), 5.283 (0.46), 5.320 (2.87), 5.450 (5.34), 6.624 (0.73), 6.937 (0.52), 7.040 (0.87), 7.161 (0.75), 7.172 (0.93), 7.217 (0.96), 7.232 (3.31), 7.238 (3.25), 7.283 (0.75), 7.390 (0.44), 7.492 (1.31), 7.655 (2.73), 7.693 (0.93), 7.705 (1.60), 7.718 (1.13), 7.839 (1.05), 7.852 (1.68), 7.865 (1.74), 7.965 (0.73), 7.994 (2.00), 8.009 (1.71), 8.043 (0.73), 8.092 (1.28), 8.105 (1.28), 8.192 (0.64), 8.256 (2.21), 8.270 (2.06), 8.525 (0.58), 8.826 (0.55), 8.897 (0.64), 8.966 (0.73), 9.186 (0.44), 11.304 (0.52).

### Example 4: Preparation of sodium (4S)-4,11-diethyl-3,14-dioxo-3,4,12,14-tetrahydro-1H-pyrano[3′,4′:6,7]indolizino[1,2-b]quinolin-4-yl 1-{(2S)-2-(carboxylatomethyl)-4,17,20-trioxo-20-[(5-sulfamoyl-1,3,4-thiadiazol-2-yl)amino]-7,10,13-trioxa-3,16-diazaicosanan-1-oyl}-L-prolyl-L-valinate (Compound 4)

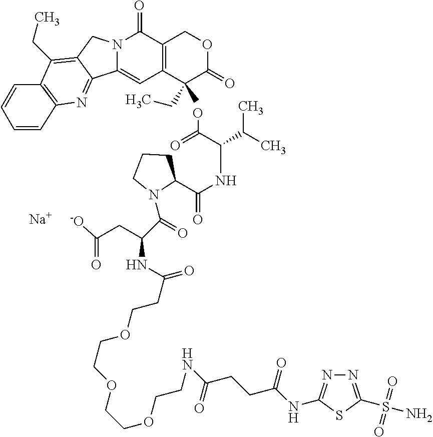

Step 1: To a solution of (4S)-4,11-diethyl-3,14-dioxo-3,4,12,14-tetrahydro-1H-pyrano[3′,4′:6,7]indolizino[1,2-b]quinolin-4-yl N-(3-{2-[2-(2-aminoethoxy)ethoxy]ethoxy}propanoyl)-L-alpha-aspartyl-L-prolyl-L-valinate.trifluoroacetic acid (1/1) (22.0 mg, 21.9 μmol) (Intermediate 6) in DMF (4 ml), were added 4-oxo-4-[(5-sulfamoyl-1,3,4-thiadiazol-2-yl)amino]butanoic acid (7.98 mg, 28.5 μmol, CAS Nr: 78851-85-1), HATU (12.5 mg, 32.8 μmol) and DIEA (11 μl, 66 μmol). The mixture was stirred at rt for 2 h and then concentrated under reduced pressure. The residue was purified over preparative HPLC and freeze dried to afford (4S)-4,11-diethyl-3,14-dioxo-3,4,12,14-tetrahydro-1H-pyrano[3′,4′:6,7]indolizino[1,2-b]quinolin-4-yl N-{14,17-dioxo-17-[(5-sulfamoyl-1,3,4-thiadiazol-2-yl)amino]-4,7,10-trioxa-13-azaheptadecanan-1-oyl}-L-alpha-aspartyl-L-prolyl-L-valinate (2.0 mg, 100% purity, 8% yield) as a yellow foam. LC-MS (Method 3): Rₜ=3.54 min; MS (ESIpos): m/z=1153 [M+H].

Step 2: To a solution of (4S)-4,11-diethyl-3,14-dioxo-3,4,12,14-tetrahydro-1H-pyrano[3′,4′:6,7]indolizino[1,2-b]quinolin-4-yl N-{14,17-dioxo-17-[(5-sulfamoyl-1,3,4-thiadiazol-2-yl)amino]-4,7,10-trioxa-13-azaheptadecanan-1-oyl}-L-alpha-aspartyl-L-prolyl-L-valinate (2.00 mg, 1.73 μmol) in dioxane/water (1:1, 2 mL) was added a sodium hydroxide solution (17 μl, 0.1 M, 1.7 μmol). The solution was freeze-dried to give Compound 4 (1.42 mg, 93% purity, 64% yield) as a colorless foam. LC-MS (Method 3): Rₜ=3.54 min; MS (ESIpos): m/z=1152 [M+H]⁺.

### Example 5: Preparation of tetrasodium (4S)-4,11-diethyl-3,14-dioxo-3,4,12,14-tetrahydro-1H-pyrano[3′,4′:6,7]indolizino[1,2-b]quinolin-4-yl 1-[(2S,23S,27S)-23,27,29-tricarboxylato-2-(carboxylatomethyl)-4,17,25-trioxo-7,10,13-trioxa-3,16,18,24,26-pentaazanonacosanan-1-oyl]-L-prolyl-L-valinate (Compound 5)

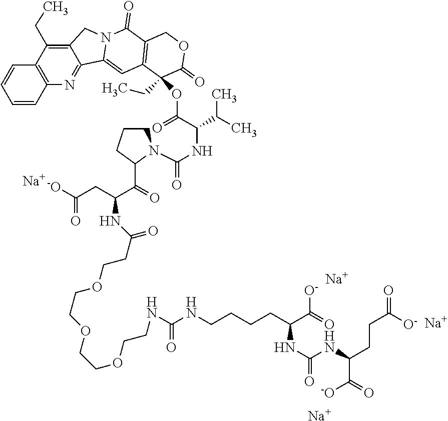

Step 1: To a solution of (4S)-4,11-diethyl-3,14-dioxo-3,4,12,14-tetrahydro-1H-pyrano[3′,4′:6,7]indolizino[1,2-b]quinolin-4-yl N-[(3S,7S)-1,3,7-tricarboxy-5,13,26-trioxo-17,20,23-trioxa-4,6,12,14-tetraazahexacosan-26-yl]-L-alpha-aspartyl-L-prolyl-L-valinate (Intermediate 6) in DMF (10 ml), were added (di-tert-butyl N-{[(2S)-1-tert-butoxy-6-{[(4-nitrophenoxy)carbonyl]amino}-1-oxohexan-2-yl]carbamoyl}-L-glutamate (39.0 mg, 59.7 μmol, CAS Nr: 1610413-98-3, as described in WO2014078484) and DIEA (26 μl, 150 μmol). The mixture was stirred at rt for 2 h and then concentrated under reduced pressure. The residue was purified over preparative HPLC to afford (7S,11S,32S)-7,11-bis(tert-butoxycarbonyl)-32-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-2,2-dimethyl-4,9,17,30-tetraoxo-3,21,24,27-tetraoxa-8,10,16,18,31-pentaazatetratriacontan-34-oic acid (50 mg, 100% purity, 72% yield) as a yellow foam. LC-MS (Method 3): Rₜ=5.04 min; MS (ESIpos): m/z=1404 [M+H]⁺

Step 2: To a solution of (7S,11S,32S)-7,11-bis(tert-butoxycarbonyl)-32-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-2,2-dimethyl-4,9,17,30-tetraoxo-3,21,24,27-tetraoxa-8,10,16,18,31-pentaazatetratriacontan-34-oic acid (50.0 mg, 35.6 μmol) in DCM (5 ml), was added TFA (5 ml). The reaction mixture was stirred for 1 h and concentrated in vacuo. The residue was dissolved in ACN/H₂O and lyophilized to give (4S)-4,11-diethyl-3,14-dioxo-3,4,12,14-tetrahydro-1H-pyrano[3′,4′:6,7]indolizino[1,2-b]quinolin-4-yl N-[(3S,7S)-1,3,7-tricarboxy-5,13,26-trioxo-17,20,23-trioxa-4,6,12,14-tetraazahexacosan-26-yl]-L-alpha-aspartyl-L-prolyl-L-valinate (39 mg, 97% purity, 86% yield) as a colorless foam. LC-MS (Method 3): Rₜ=3.40 min; MS (ESIpos): m/z=1237 [M+H]⁺.

Step 3: To a solution of (4S)-4,11-diethyl-3,14-dioxo-3,4,12,14-tetrahydro-1H-pyrano[3′,4′:6,7]indolizino[1,2-b]quinolin-4-yl N-[(3S,7S)-1,3,7-tricarboxy-5,13,26-trioxo-17,20,23-trioxa-4,6,12,14-tetraazahexacosan-26-yl]-L-alpha-aspartyl-L-prolyl-L-valinate (39.0 mg, 31.5 μmol) in dioxane/water (1:1, 10 mL) was added a sodium hydroxide solution (130 μl, 1 M, 130 μmol). The solution was freeze-dried to give Compound 5 (42 mg, 99% purity, 100% yield) as a colorless foam. LC-MS (Method 3): Rₜ=3.39 min; MS (ESIpos): m/z=1232 [M+H]⁺. ¹H-NMR (400 MHz, DMSO-d6) delta [ppm]: 1H-NMR (400 MHz, DMSO-d6) delta [ppm]: 0.779 (0.32), 0.791 (0.32), 0.798 (0.33), 0.803 (0.34), 0.823 (0.37), 0.836 (0.51), 0.873 (3.88), 0.892 (8.04), 0.908 (8.96), 0.923 (7.89), 0.931 (7.80), 0.948 (6.27), 0.983 (0.62), 0.989 (0.60), 1.000 (0.69), 1.011 (0.81), 1.018 (0.75), 1.046 (0.33), 1.054 (0.30), 1.058 (0.30), 1.066 (0.31), 1.076 (0.32), 1.083 (0.34), 1.089 (0.31), 1.094 (0.32), 1.097 (0.31), 1.107 (0.30), 1.118 (0.32), 1.123 (0.31), 1.128 (0.32), 1.133 (0.37), 1.137 (0.39), 1.148 (0.47), 1.154 (0.50), 1.157 (0.52), 1.180 (0.85), 1.195 (1.09), 1.208 (1.30), 1.235 (2.14), 1.243 (1.98), 1.247 (2.00), 1.252 (2.05), 1.290 (5.16), 1.308 (7.08), 1.327 (3.78), 1.356 (0.62), 1.367 (0.46), 1.376 (0.39), 1.387 (0.41), 1.392 (0.41), 1.397 (0.41), 1.404 (0.44), 1.409 (0.48), 1.420 (0.63), 1.428 (0.71), 1.450 (0.89), 1.457 (0.91), 1.467 (0.91), 1.474 (0.80), 1.482 (0.72), 1.524 (0.38), 1.529 (0.38), 1.540 (0.44), 1.547 (0.55), 1.577 (0.96), 1.591 (1.05), 1.602 (1.09), 1.608 (1.09), 1.623 (0.99), 1.638 (0.94), 1.644 (0.88), 1.695 (0.42), 1.704 (0.36), 1.706 (0.36), 1.715 (0.34), 1.723 (0.37), 1.727 (0.38), 1.730 (0.38), 1.750 (0.45), 1.754 (0.51), 1.789 (0.93), 1.792 (0.95), 1.801 (0.97), 1.805 (1.01), 1.812 (0.99), 1.817 (1.01), 1.825 (0.98), 1.830 (0.99), 1.834 (0.99), 1.849 (1.08), 1.856 (1.06), 1.869 (1.09), 1.872 (1.10), 1.875 (1.11), 1.877 (1.09), 1.879 (1.08), 1.884 (1.11), 1.889 (1.13), 1.893 (1.20), 1.898 (1.23), 1.902 (1.27), 1.915 (1.36), 1.924 (1.35), 1.953 (0.96), 1.964 (1.07), 1.967 (1.09), 1.988 (1.24), 1.993 (1.26), 2.001 (1.23), 2.003 (1.19), 2.032 (0.56), 2.037 (0.55), 2.066 (0.94), 2.096 (1.65), 2.106 (1.79), 2.146 (1.09), 2.159 (1.03), 2.167 (1.04), 2.181 (1.40), 2.199 (2.67), 2.216 (3.70), 2.235 (3.71), 2.285 (1.65), 2.303 (2.16), 2.321 (1.93), 2.326 (1.91), 2.332 (1.94), 2.338 (2.01), 2.362 (1.85), 2.379 (1.33), 2.399 (1.07), 2.408 (0.75), 2.414 (0.77), 2.433 (0.56), 2.471 (1.04), 2.570 (0.32), 2.583 (0.39), 2.606 (0.85), 2.610 (0.80), 2.625 (0.97), 2.641 (0.89), 2.669 (1.00), 2.690 (0.39), 2.702 (0.35), 2.706 (0.34), 2.716 (0.43), 2.740 (0.39), 2.745 (0.44), 2.751 (0.39), 2.764 (0.39), 2.769 (0.38), 2.782 (0.39), 2.792 (0.41), 2.797 (0.44), 2.819 (0.47), 2.827 (0.48), 2.846 (0.53), 2.868 (0.69), 2.919 (2.49), 2.955 (1.04), 2.960 (0.95), 2.963 (0.92), 2.968 (0.87), 2.977 (0.85), 2.982 (0.81), 2.990 (0.81), 3.000 (0.82), 3.016 (0.83), 3.026 (0.92), 3.031 (0.95), 3.053 (1.16), 3.105 (3.40), 3.138 (1.69), 3.150 (1.71), 3.174 (2.08), 3.211 (3.52), 3.215 (3.60), 3.231 (3.76), 3.242 (3.42), 3.337 (9.80), 3.353 (9.73), 3.569 (16.00), 3.590 (6.48), 3.608 (5.01), 3.686 (1.65), 3.689 (1.60), 3.698 (1.46), 3.718 (1.27), 3.742 (1.11), 3.748 (1.08), 3.759 (1.09), 3.789 (1.64), 3.814 (2.21), 3.818 (2.19), 3.838 (1.80), 3.862 (1.88), 3.883 (2.60), 3.903 (2.57), 3.925 (1.77), 3.966 (0.52), 3.984 (0.32), 3.986 (0.30), 3.995 (0.31), 4.815 (1.11), 4.830 (1.07), 4.832 (1.08), 4.847 (0.55), 5.088 (1.09), 5.095 (0.99), 5.097 (0.96), 5.103 (1.09), 5.267 (0.31), 5.274 (0.43), 5.298 (0.52), 5.326 (3.39), 5.376 (0.31), 5.436 (0.31), 5.469 (5.15), 6.130 (0.41), 6.177 (1.74), 6.183 (1.71), 6.217 (0.82), 6.227 (0.75), 6.247 (0.76), 6.251 (0.81), 6.262 (0.78), 6.269 (0.78), 6.278 (0.71), 7.514 (2.68), 7.705 (0.97), 7.723 (1.60), 7.742 (1.09), 7.849 (1.15), 7.868 (1.70), 7.885 (1.02), 8.019 (2.38), 8.040 (1.74), 8.220 (1.35), 8.238 (1.30), 8.265 (1.84), 8.286 (1.50), 8.741 (0.92), 8.757 (0.88), 8.759 (0.88).

### Example 6: Preparation of sodium (4S)-4,11-diethyl-3,14-dioxo-3,4,12,14-tetrahydro-1H-pyrano[3′,4′:6,7]indolizino[1,2-b]quinolin-4-yl 1-[(2S)-2-(carboxylatomethyl)-20-{[4-({2-[(2S)-2-cyano-4,4-difluoropyrrolidin-1-yl]-2-oxoethyl}carbamoyl)quinolin-8-yl]amino}-4,17,20-trioxo-7,10,13-trioxa-3,16-diazaicosanan-1-oyl]-L-prolyl-L-valinate (Compound 6)

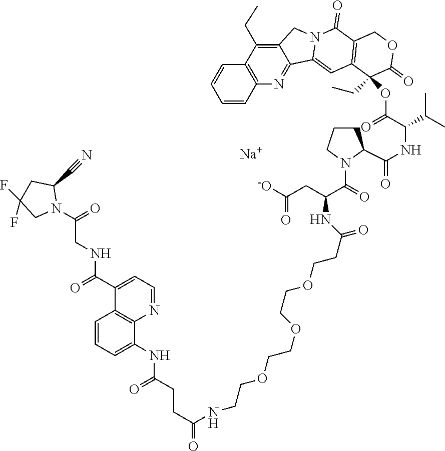

Step 1: To a solution of 4-{[4-({2-[(2S)-2-cyano-4,4-difluoropyrrolidin-1-yl]-2-oxoethyl}carbamoyl)quinolin-8-yl]amino}-4-oxobutanoic acid (20.0 mg, 43.5 μmol) in DMF (1 ml) and DCM (1 ml), were added HATU (16.6 mg, 43.5 μmol) and DIEA (30 μl, 170 μmol). After stirring for 15 min, (4S)-4,11-diethyl-3,14-dioxo-3,4,12,14-tetrahydro-1H-pyrano[3′,4′:6,7]indolizino[1,2-b]quinolin-4-yl N-(3-{2-[2-(2-aminoethoxy)ethoxy]ethoxy}propanoyl)-L-alpha-aspartyl-L-prolyl-L-valinate.trifluoroacetic acid (1/1) (43.8 mg, 43.5 μmol) (Intermediate 6) was added. The mixture was stirred at rt for 1h and then concentrated under reduced pressure. The residue was purified over preparative HPLC and freeze dried to afford ((4S)-4,11-diethyl-3,14-dioxo-3,4,12,14-tetrahydro-1H-pyrano[3′,4′:6,7]indolizino[1,2-b]quinolin-4-yl N-(17-{[4-({2-[(2S)-2-cyano-4,4-difluoropyrrolidin-1-yl]-2-oxoethyl}carbamoyl)quinolin-8-yl]amino}-14,17-dioxo-4,7,10-trioxa-13-azaheptadecanan-1-oyl}-L-alpha-aspartyl-L-prolyl-L-valinate (29.5 mg, 100% purity, 51% yield). LC-MS (Method 3): Rₜ=3.95 min; MS (ESIpos): m/z=1333 [M+H]⁺.

Step 2: To a solution of (4S)-4,11-diethyl-3,14-dioxo-3,4,12,14-tetrahydro-1H-pyrano[3′,4′:6,7]indolizino[1,2-b]quinolin-4-yl N-(17-{[4-({2-[(2S)-2-cyano-4,4-difluoropyrrolidin-1-yl]-2-oxoethyl}carbamoyl)quinolin-8-yl]amino}-14,17-dioxo-4,7,10-trioxa-13-azaheptadecanan-1-oyl}-L-alpha-aspartyl-L-prolyl-L-valinate (29.2 mg, 21.9 μmol) in dioxane/water (1:1, 6 mL) was added a sodium hydroxide solution (48 μl, 1 M, 48 μmol). The solution was freeze-dried to give Compound 6 (29.1 mg, 100% purity, 98% yield) as a colorless foam. LC-MS (Method 3): Rₜ=3.95 min; MS (ESIpos): m/z=1333 [M+H]⁺. ¹H-NMR (400 MHz, DMSO-d6) δ[ppm]: 0.841 (2.08), 0.860 (7.04), 0.877 (5.54), 0.914 (3.62), 0.930 (3.77), 1.283 (2.03), 1.302 (4.11), 1.321 (2.16), 1.764 (0.50), 1.775 (0.48), 1.780 (0.52), 1.789 (0.51), 1.906 (0.51), 1.912 (0.59), 1.930 (0.81), 1.941 (0.90), 1.952 (0.83), 1.965 (0.89), 2.011 (0.44), 2.032 (1.05), 2.046 (0.64), 2.059 (1.21), 2.070 (0.95), 2.211 (0.59), 2.227 (1.26), 2.244 (1.56), 2.258 (1.42), 2.278 (1.32), 2.294 (0.83), 2.318 (0.94), 2.335 (1.23), 2.352 (0.78), 2.371 (0.58), 2.618 (0.55), 2.645 (0.60), 2.655 (0.60), 2.664 (0.56), 2.669 (0.60), 2.676 (0.52), 2.682 (0.59), 2.763 (1.35), 2.780 (2.52), 2.798 (1.46), 2.813 (0.88), 2.848 (0.65), 2.868 (0.48), 2.938 (0.48), 3.121 (0.59), 3.137 (0.68), 3.149 (0.87), 3.157 (1.03), 3.170 (2.26), 3.184 (2.71), 3.197 (1.78), 3.211 (1.74), 3.231 (1.34), 3.245 (0.97), 3.379 (2.76), 3.388 (3.72), 3.399 (2.48), 3.406 (2.66), 3.423 (16.00), 3.446 (2.41), 3.471 (0.70), 3.478 (1.02), 3.491 (0.70), 3.518 (1.37), 3.526 (1.61), 3.539 (4.68), 3.566 (4.28), 3.577 (1.35), 3.592 (1.12), 3.673 (0.70), 3.694 (1.10), 3.714 (0.64), 3.803 (0.59), 3.827 (0.83), 3.835 (0.68), 3.845 (0.78), 3.995 (0.43), 4.012 (0.74), 4.117 (0.49), 4.148 (0.51), 4.177 (0.55), 4.198 (0.45), 4.230 (1.67), 4.242 (1.65), 4.284 (0.44), 4.291 (0.49), 4.322 (0.63), 4.632 (0.46), 4.638 (0.47), 4.650 (0.48), 4.655 (0.47), 4.747 (0.47), 4.753 (0.59), 4.762 (0.66), 4.769 (0.56), 4.774 (0.49), 5.168 (0.89), 5.173 (0.94), 5.190 (0.96), 5.282 (1.01), 5.291 (0.54), 5.314 (2.50), 5.319 (2.52), 5.443 (3.89), 7.593 (0.76), 7.613 (1.48), 7.634 (1.00), 7.643 (1.80), 7.654 (1.95), 7.690 (0.70), 7.713 (3.91), 7.728 (1.01), 7.830 (0.72), 7.849 (1.18), 7.867 (0.80), 7.965 (1.55), 7.977 (1.93), 7.986 (1.60), 7.997 (1.47), 8.050 (1.12), 8.069 (1.17), 8.113 (0.65), 8.128 (1.25), 8.142 (0.70), 8.254 (1.32), 8.276 (1.20), 8.520 (1.18), 8.623 (1.41), 8.642 (1.36), 8.989 (1.94), 8.999 (1.85), 9.176 (0.88), 9.194 (0.94), 9.247 (0.56), 9.261 (1.05), 9.274 (0.63), 10.137 (2.29).

### Example 7: Preparation of heptasodium (4S)-4,11-diethyl-3,14-dioxo-3,4,12,14-tetrahydro-1H-pyrano[3′,4′:6,7]indolizino[1,2-b]quinolin-4-yl 1-[(2S)-15-{[N2,N6-bis(3S,7S)(1,3,7-tricarboxylato-5,13,26-trioxo-17,20,23-trioxa-4,6,12,14-tetraazahexacosan-26-yl)-L-lysyl]amino}-2-(carboxylatomethyl)-4-oxo-7,10,13-trioxa-3-azapentadecanan-1-oyl]-L-prolyl-L-valinate (Compound 7)

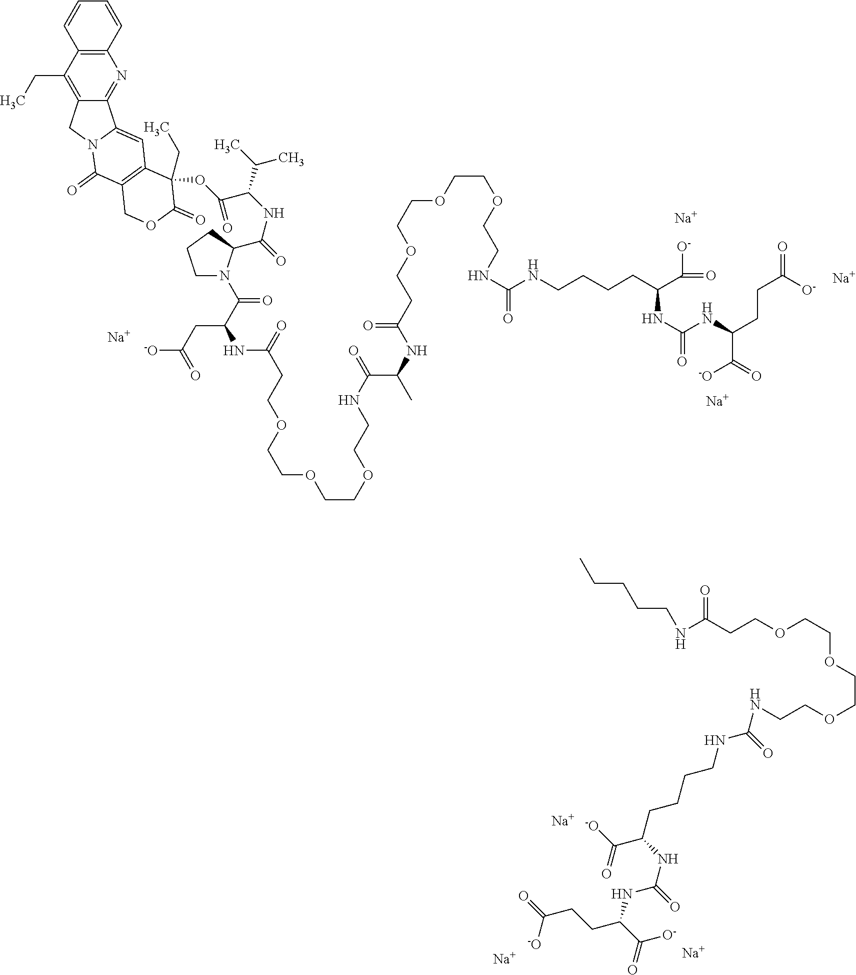

Step 1: To a solution of (4S)-4,11-diethyl-3,14-dioxo-3,4,12,14-tetrahydro-1H-pyrano[3′,4′:6,7]indolizino[1,2-b]quinolin-4-yl N-(3-{2-[2-(2-{[N²,N⁶-bis(3-{2-[2-(2-aminoethoxy)ethoxy]ethoxy}propanoyl)-L-lysyl]amino}ethoxy)ethoxy]ethoxy}propanoyl)-L-alpha-aspartyl-L-prolyl-L-valinate.trifluoroacetic acid (1/2) (25.0 mg, 90% purity, 13.6 μmol)

(Intermediate 9) in DMF (3.8 ml), were added (di-tert-butyl N-{[(2S)-1-tert-butoxy-6-{[(4-nitrophenoxy)carbonyl]amino}-1-oxohexan-2-yl]carbamoyl}-L-glutamate (39.0 mg, 59.7 μmol, CAS Nr: 1610413-98-3, as described in WO2014078484) and DIEA (7.1 μl, 41 μmol). The mixture was stirred at rt for 4 h and then concentrated under reduced pressure. The residue was purified over preparative HPLC to afford (7S,11S,36S,52S)-36-{[(7S,11S)-7,11-bis(tert-butoxycarbonyl)-2,2-dimethyl-4,9,17,30-tetraoxo-3,21,24,27-tetraoxa-8,10,16,18-tetraazatriacontan-30-yl]amino}-7,11-bis(tert-butoxycarbonyl)-52-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-2,2-dimethyl-4,9,17,30,37,50-hexaoxo-3,21,24,27,41,44,47-heptaoxa-8,10,16,18,31,38,51-heptaazatetrapentacontan-54-oic acid (16 mg, 96% purity, 46% yield) as a colorless foam. LC-MS (Method 3): Rₜ=5.71 min; MS (ESIpos): m/z=2452 [M+H]⁺

Step 2: To a solution of (7S,11S,36S,52S)-36-{[(7S,11S)-7,11-bis(tert-butoxycarbonyl)-2,2-dimethyl-4,9,17,30-tetraoxo-3,21,24,27-tetraoxa-8,10,16,18-tetraazatriacontan-30-yl]amino}-7,11-bis(tert-butoxycarbonyl)-52-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-2,2-dimethyl-4,9,17,30,37,50-hexaoxo-3,21,24,27,41,44,47-heptaoxa-8,10,16,18,31,38,51-heptaazatetrapentacontan-54-oic acid (16.0 mg, 6.52 μmol) in DCM (3 ml), was added TFA (3 ml). The reaction mixture was stirred for 4 h and concentrated in vacuo. The residue was dissolved in ACN/H₂O and lyophilized to give (4S)-4,11-diethyl-3,14-dioxo-3,4,12,14-tetrahydro-1H-pyrano[3′,4′:6,7]indolizino[1,2-b]quinolin-4-yl N-(3-{2-[2-{2-([N²,N⁶-bis(3S,7S)(1,3,7-tricarboxy-5,13,26-trioxo-17,20,23-trioxa-4,6,12,14-tetraazahexacosan-26-yl)-L-lysyl]amino}ethoxy)ethoxy]ethoxy}propanoyl)-L-alpha-aspartyl-L-prolyl-L-valinate (13 mg, 95% purity, 89% yield) as a yellow foam. LC-MS (Method 3): Rₜ=3.33 min; MS (ESIpos): m/z=2115 [M+H]⁺

Step 3: To a solution of (4S)-4,11-diethyl-3,14-dioxo-3,4,12,14-tetrahydro-1H-pyrano[3′,4′:6,7]indolizino[1,2-b]quinolin-4-yl N-(3-{2-[2-(2-{[N²,N⁶-bis(3S,7S)(1,3,7-tricarboxy-5,13,26-trioxo-17,20,23-trioxa-4,6,12,14-tetraazahexacosan-26-yl)-L-lysyl]amino}ethoxy)ethoxy]ethoxy}propanoyl)-L-alpha-aspartyl-L-prolyl-L-valinate (13.0 mg, 6.14 μmol) in dioxane/water (1:1, 3 mL) was added a sodium hydroxide solution (43 μl, 1 M, 43 μmol). The solution was freeze-dried to give Compound 7 (14 mg, 95% purity, 95% yield) as a colorless foam. LC-MS (Method 3): R: =3.29 min; MS (ESIpos): m/z=2108 [M+H]⁺. ¹H-NMR (400 MHz, DMSO-d6) delta [ppm]: 0.787 (0.16), 0.801 (0.24), 0.810 (0.20), 0.825 (0.22), 0.869 (0.98), 0.887 (2.04), 0.901 (1.93), 0.905 (1.75), 0.917 (1.89), 0.927 (3.20), 0.942 (16.00), 0.945 (7.68), 0.958 (15.29), 0.962 (3.48), 0.976 (0.33), 0.992 (0.18), 1.000 (0.22), 1.007 (0.24), 1.015 (0.24), 1.023 (0.22), 1.041 (0.20), 1.055 (0.22), 1.087 (0.16), 1.097 (0.18), 1.111 (0.18), 1.119 (0.22), 1.135 (0.16), 1.140 (0.18), 1.152 (0.44), 1.157 (0.36), 1.171 (0.87), 1.189 (0.75), 1.198 (0.64), 1.202 (0.73), 1.230 (1.13), 1.235 (1.46), 1.244 (2.09), 1.260 (2.04), 1.275 (2.26), 1.292 (2.75), 1.314 (3.00), 1.332 (1.69), 1.369 (0.47), 1.379 (0.40), 1.384 (0.33), 1.393 (0.27), 1.404 (0.27), 1.412 (0.24), 1.432 (0.36), 1.443 (0.44), 1.485 (0.64), 1.495 (0.53), 1.508 (0.40), 1.522 (0.29), 1.534 (0.31), 1.540 (0.36), 1.547 (0.47), 1.565 (0.82), 1.569 (0.84), 1.574 (0.87), 1.583 (0.95), 1.588 (1.00), 1.592 (1.00), 1.597 (0.91), 1.601 (0.87), 1.630 (0.42), 1.640 (0.27), 1.659 (0.18), 1.669 (0.20), 1.673 (0.18), 1.681 (0.16), 1.690 (0.20), 1.765 (0.22), 1.770 (0.27), 1.772 (0.24), 1.786 (0.47), 1.793 (0.47), 1.798 (0.44), 1.806 (0.53), 1.828 (0.53), 1.843 (0.51), 1.861 (0.40), 1.889 (0.29), 1.907 (0.33), 1.910 (0.38), 1.914 (0.33), 1.920 (0.33), 1.927 (0.38), 1.935 (0.38), 1.950 (0.36), 1.953 (0.33), 1.957 (0.36), 1.961 (0.36), 1.964 (0.36), 1.970 (0.36), 1.978 (0.33), 1.984 (0.33), 2.007 (0.42), 2.013 (0.38), 2.055 (0.24), 2.072 (0.38), 2.082 (0.36), 2.085 (0.36), 2.096 (0.40), 2.102 (0.44), 2.105 (0.49), 2.118 (0.60), 2.123 (0.60), 2.145 (0.69), 2.159 (0.75), 2.192 (0.47), 2.196 (0.49), 2.201 (0.49), 2.215 (1.00), 2.219 (1.00), 2.233 (1.33), 2.248 (0.89), 2.253 (0.89), 2.267 (1.00), 2.278 (1.24), 2.287 (1.04), 2.294 (1.69), 2.311 (1.20), 2.320 (0.95), 2.326 (1.26), 2.331 (1.60), 2.335 (1.44), 2.339 (0.98), 2.352 (1.02), 2.359 (0.75), 2.369 (1.73), 2.385 (0.78), 2.398 (0.82), 2.406 (1.07), 2.414 (0.67), 2.424 (1.95), 2.434 (0.71), 2.441 (1.93), 2.449 (0.58), 2.459 (1.13), 2.465 (0.93), 2.473 (1.09), 2.572 (0.27), 2.586 (0.20), 2.591 (0.22), 2.599 (0.40), 2.624 (0.20), 2.633 (0.27), 2.644 (0.36), 2.663 (0.64), 2.668 (1.00), 2.673 (1.22), 2.677 (0.98), 2.682 (0.71), 2.704 (0.40), 2.713 (0.93), 2.734 (0.29), 2.744 (0.24), 2.748 (0.29), 2.762 (0.33), 2.768 (0.33), 2.771 (0.31), 2.775 (0.29), 2.786 (0.33), 2.790 (0.33), 2.811 (0.33), 2.823 (0.42), 2.828 (0.42), 2.833 (0.40), 2.848 (0.47), 2.858 (0.42), 2.868 (0.44), 2.876 (0.49), 2.894 (0.78), 2.918 (1.73), 2.930 (1.66), 2.941 (1.55), 2.958 (1.78), 2.974 (2.22), 2.991 (1.98), 3.001 (1.07), 3.007 (1.22), 3.022 (0.87), 3.029 (0.89), 3.033 (0.89), 3.048 (0.93), 3.056 (0.95), 3.059 (1.00), 3.069 (1.07), 3.104 (2.49), 3.117 (2.66), 3.140 (1.95), 3.162 (2.33), 3.168 (2.49), 3.177 (2.66), 3.194 (2.71), 3.200 (2.77), 3.224 (3.51), 3.244 (4.31), 3.246 (4.28), 3.529 (4.93), 3.559 (4.33), 3.570 (9.94), 3.636 (1.35), 3.645 (1.20), 3.650 (1.20), 3.660 (1.11), 3.668 (1.02), 3.674 (0.98), 3.678 (1.02), 3.679 (1.00), 3.686 (0.91), 3.689 (0.89), 3.711 (0.80), 3.719 (0.69), 3.723 (0.67), 3.726 (0.64), 3.741 (0.60), 3.761 (0.51), 3.767 (0.53), 3.777 (0.49), 3.795 (0.49), 3.800 (0.55), 3.805 (0.53), 3.817 (0.67), 3.828 (1.02), 3.848 (1.51), 3.860 (0.98), 3.868 (0.93), 3.885 (0.49), 3.901 (0.49), 3.924 (0.69), 3.942 (0.84), 3.979 (0.27), 3.986 (0.16), 3.995 (0.20), 3.999 (0.16), 4.141 (0.20), 4.154 (0.29), 4.163 (0.38), 4.170 (0.27), 4.177 (0.38), 4.182 (0.29), 4.783 (0.09), 4.790 (0.11), 4.793 (0.16), 4.797 (0.16), 4.815 (0.36), 4.846 (0.13), 5.127 (0.33), 5.143 (0.29), 5.149 (0.31), 5.319 (0.24), 5.334 (1.04), 5.351 (0.20), 5.465 (1.64), 6.095 (0.18), 6.100 (0.16), 6.126 (0.60), 6.139 (1.00), 6.162 (0.49), 6.171 (0.24), 6.182 (0.18), 6.246 (0.13), 6.257 (0.18), 6.263 (0.22), 6.271 (0.20), 6.281 (0.29), 6.287 (0.29), 6.292 (0.22), 6.300 (0.22), 6.305 (0.24), 6.319 (0.27), 6.328 (0.20), 6.332 (0.20), 6.340 (0.24), 6.344 (0.22), 6.350 (0.31), 6.362 (0.42), 6.372 (0.42), 6.381 (0.36), 7.501 (0.07), 7.520 (0.09), 7.543 (1.11), 7.691 (0.07), 7.711 (0.29), 7.728 (0.44), 7.732 (0.40), 7.749 (0.31), 7.761 (0.09), 7.850 (0.29), 7.865 (0.42), 7.868 (0.51), 7.896 (0.11), 7.936 (0.27), 7.950 (0.42), 7.963 (0.22), 8.011 (0.69), 8.024 (0.13), 8.033 (0.42), 8.128 (0.20), 8.143 (0.27), 8.148 (0.40), 8.164 (0.20), 8.213 (0.24), 8.219 (0.33), 8.274 (0.60), 8.294 (0.51), 8.310 (−0.09), 8.339 (0.31), 8.357 (0.29), 8.712 (0.22), 8.720 (0.20).

### Example 8: Preparation of sodium (4S)-4,11-diethyl-3,14-dioxo-3,4,12,14-tetrahydro-1H-pyrano[3′,4′:6,7]indolizino[1,2-b]quinolin-4-yl 1-[(2S)-15-[(N2,N6-bis{14,17-dioxo-17-[(5-sulfamoyl-1,3,4-thiadiazol-2-yl)amino]-4,7,10-trioxa-13-azaheptadecanan-1-oyl}-L-lysyl)amino]-2-(carboxylatomethyl)-4-oxo-7,10,13-trioxa-3-azapentadecanan-1-oyl]-L-prolyl-L-valinate (Compound 8)

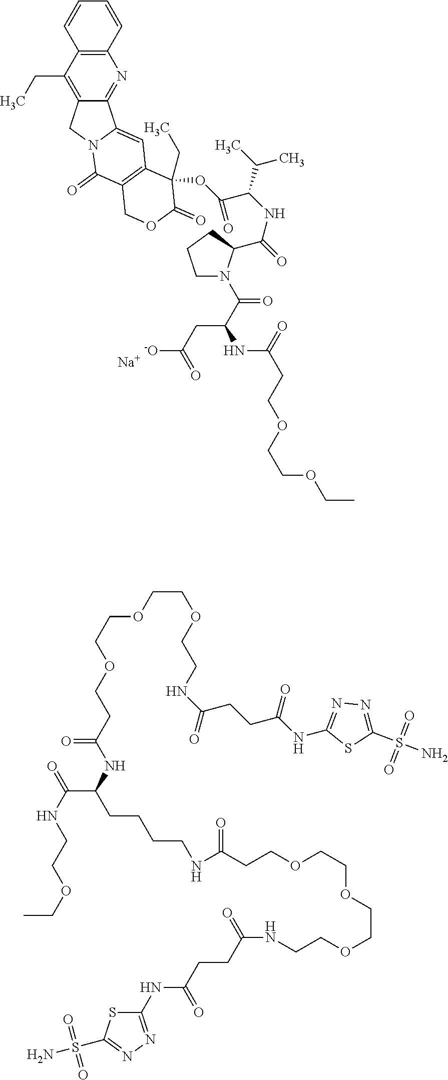

Step 1: To a solution of (4S)-4,11-diethyl-3,14-dioxo-3,4,12,14-tetrahydro-1H-pyrano[3′,4′:6,7]indolizino[1,2-b]quinolin-4-yl N-(3-{2-[2-(2-{[N²,N⁶-bis(3-{2-[2-(2-aminoethoxy)ethoxy]ethoxy}propanoyl)-L-lysyl]amino}ethoxy)ethoxy]ethoxy}propanoyl)-L-alpha-aspartyl-L-prolyl-L-valinate.trifluoroacetic acid (1/2) (15.0 mg, 9.07 μmol) (Intermediate 9) in DMF (2 ml), were added pentafluorophenyl 4-oxo-4-[(5-sulfamoyl-1,3,4-thiadiazol-2-yl)amino]butanoate (32.4 mg, 72.6 μmol) (Intermediate 19) and DIEA (13 μl, 73 μmol). The mixture was stirred at rt for 1 h and then concentrated under reduced pressure. The residue was purified over preparative HPLC and freeze dried to afford (4S)-4,11-diethyl-3,14-dioxo-3,4,12,14-tetrahydro-1H-pyrano[3′,4′:6,7]indolizino[1,2-b]quinolin-4-yl N-{3-[2-(2-{2-[(N2,N6-bis(14,17-dioxo-17-[(5-sulfamoyl-1,3,4-thiadiazol-2-yl)amino]-4,7,10-trioxa-13-azaheptadecanan-1-oyl}-L-lysyl)amino]ethoxy)ethoxy)ethoxy]propanoyl}-L-alpha-aspartyl-L-prolyl-L-valinate (9.2 mg, 98% purity, 51% yield) as a yellow foam. LC-MS (Method 3): Rₜ=3.52 min; MS (ESIpos): m/z=1949 [M+H]⁺

Step 2: To a solution of (4S)-4,11-diethyl-3,14-dioxo-3,4,12,14-tetrahydro-1H-pyrano[3′,4′:6,7]indolizino[1,2-b]quinolin-4-yl N-{3-[2-(2-{2-[{N2,N6-bis(14,17-dioxo-17-[(5-sulfamoyl-1,3,4-thiadiazol-2-yl)amino]-4,7,10-trioxa-13-azaheptadecanan-1-oyl}-L-lysyl)amino]ethoxy}ethoxy)ethoxy]propanoyl}-L-alpha-aspartyl-L-prolyl-L-valinate (9.20 mg, 4.72 μmol) in dioxane/water (1:1, 3 mL) was added a sodium hydroxide solution (47 μl, 1 M, 47 μmol). The solution was freeze-dried to give Compound 8 (9 mg, 96% purity, 93% yield) as a colorless foam. LC-MS (Method 3): Rₜ=3.53 min; MS (ESIpos): m/z=1948 [M+H]⁺. ¹H-NMR (400 MHz, DMSO-d6) delta [ppm]: −0.062 (0.15), 0.069 (0.10), 0.825 (0.11), 0.830 (0.23), 0.847 (0.25), 0.852 (0.13), 0.869 (0.12), 0.891 (0.58), 0.909 (1.26), 0.928 (0.74), 0.940 (1.74), 0.956 (1.72), 0.995 (0.13), 1.000 (0.09), 1.012 (0.15), 1.146 (0.07), 1.161 (0.09), 1.166 (0.09), 1.178 (0.13), 1.194 (0.17), 1.202 (0.20), 1.208 (0.20), 1.217 (0.22), 1.235 (0.37), 1.261 (0.14), 1.271 (0.13), 1.292 (0.65), 1.312 (1.44), 1.330 (0.91), 1.347 (0.37), 1.361 (0.24), 1.366 (0.23), 1.404 (0.07), 1.421 (0.08), 1.430 (0.10), 1.465 (0.16), 1.478 (0.14), 1.496 (0.08), 1.502 (0.08), 1.544 (0.08), 1.546 (0.08), 1.567 (0.13), 1.578 (0.15), 1.581 (0.15), 1.588 (0.13), 1.619 (0.08), 1.892 (0.12), 1.903 (0.16), 1.909 (0.19), 1.919 (0.24), 1.935 (0.25), 1.956 (0.17), 1.974 (0.10), 1.978 (0.10), 1.986 (0.13), 1.998 (0.16), 2.019 (0.16), 2.031 (0.17), 2.037 (0.16), 2.049 (0.10), 2.090 (0.09), 2.111 (0.16), 2.117 (0.15), 2.131 (0.18), 2.139 (0.17), 2.148 (0.16), 2.151 (0.17), 2.161 (0.25), 2.181 (0.40), 2.185 (0.39), 2.205 (0.42), 2.226 (0.29), 2.246 (0.20), 2.268 (0.47), 2.284 (0.93), 2.301 (0.55), 2.322 (0.34), 2.331 (0.54), 2.336 (0.54), 2.346 (0.52), 2.368 (0.68), 2.383 (0.66), 2.398 (0.39), 2.419 (0.37), 2.447 (0.72), 2.465 (1.59), 2.483 (1.12), 2.570 (0.07), 2.579 (0.07), 2.584 (0.08), 2.598 (0.07), 2.610 (0.08), 2.620 (0.08), 2.626 (0.08), 2.633 (0.08), 2.663 (0.87), 2.680 (1.48), 2.697 (0.65), 2.713 (0.31), 2.726 (0.25), 2.749 (0.21), 2.768 (0.20), 2.779 (0.10), 2.783 (0.10), 2.790 (0.15), 2.796 (0.10), 2.805 (0.09), 2.808 (0.09), 2.813 (0.09), 2.822 (0.09), 2.835 (0.10), 2.837 (0.10), 2.845 (0.10), 2.850 (0.10), 2.864 (0.10), 2.871 (0.10), 2.874 (0.11), 2.880 (0.11), 2.891 (0.11), 2.903 (0.12), 2.911 (0.13), 2.915 (0.12), 2.919 (0.13), 2.922 (0.14), 2.926 (0.14), 2.947 (0.25), 2.958 (0.31), 2.963 (0.30), 2.976 (0.50), 2.994 (0.54), 3.010 (0.33), 3.028 (0.23), 3.034 (0.23), 3.037 (0.23), 3.039 (0.22), 3.042 (0.23), 3.054 (0.24), 3.060 (0.24), 3.064 (0.25), 3.071 (0.26), 3.097 (0.30), 3.105 (0.33), 3.116 (0.37), 3.124 (0.46), 3.141 (0.55), 3.153 (0.69), 3.169 (1.13), 3.177 (1.98), 3.191 (2.18), 3.205 (1.45), 3.218 (1.31), 3.238 (1.50), 3.386 (16.00), 3.459 (5.96), 3.468 (8.56), 3.477 (5.68), 3.489 (7.84), 3.520 (1.45), 3.526 (1.32), 3.551 (1.65), 3.557 (2.02), 3.568 (3.73), 3.572 (2.12), 3.583 (2.48), 3.589 (2.31), 3.597 (1.60), 3.602 (2.92), 3.610 (2.56), 3.626 (0.43), 3.633 (0.82), 3.640 (0.82), 3.659 (0.29), 3.667 (0.26), 3.677 (0.24), 3.700 (0.21), 3.707 (0.20), 3.713 (0.20), 3.722 (0.22), 3.749 (0.38), 3.765 (0.40), 3.785 (0.26), 3.810 (0.19), 3.818 (0.16), 3.830 (0.15), 3.838 (0.14), 3.844 (0.13), 3.854 (0.09), 3.862 (0.08), 3.864 (0.08), 3.868 (0.08), 3.874 (0.08), 3.882 (0.15), 3.890 (0.09), 3.915 (0.07), 3.925 (0.07), 3.948 (0.07), 3.964 (0.34), 3.976 (0.30), 3.982 (0.37), 3.994 (0.53), 4.004 (0.30), 4.012 (0.36), 4.023 (0.41), 4.042 (0.39), 4.062 (0.22), 4.161 (0.13), 4.165 (0.09), 4.181 (0.22), 4.194 (0.24), 4.201 (0.18), 4.215 (0.13), 4.330 (0.16), 4.833 (0.52), 4.840 (0.99), 4.846 (0.58), 4.878 (0.26), 4.898 (0.45), 4.917 (0.27), 4.933 (0.09), 5.288 (0.09), 5.335 (0.58), 5.344 (0.57), 5.392 (0.08), 5.487 (1.19), 7.082 (0.07), 7.298 (0.88), 7.719 (0.20), 7.737 (0.35), 7.758 (0.25), 7.789 (0.19), 7.800 (0.33), 7.813 (0.19), 7.847 (0.23), 7.864 (0.35), 7.885 (0.23), 7.897 (0.09), 7.912 (0.20), 7.925 (0.35), 7.939 (0.20), 7.964 (0.46), 7.969 (0.51), 7.983 (0.97), 7.997 (0.39), 8.040 (0.45), 8.061 (0.36), 8.097 (0.07), 8.109 (0.07), 8.117 (0.07), 8.123 (0.07), 8.126 (0.07), 8.136 (0.10), 8.139 (0.11), 8.146 (0.09), 8.158 (0.10), 8.164 (0.10), 8.169 (0.08), 8.176 (0.08), 8.185 (0.08), 8.199 (0.09), 8.208 (0.09), 8.227 (0.12), 8.242 (0.28), 8.264 (0.26), 8.280 (0.44), 8.302 (0.38), 8.335 (0.33), 8.355 (0.30), 12.024 (0.12), 12.030 (0.12), 12.033 (0.11), 12.040 (0.11), 12.050 (0.09).

### Example 9: Preparation of tetrasodium 1-[(2S,23S,27S)-23,27,29-tricarboxylato-2-(carboxylatomethyl)-4,17,25-trioxo-7,10,13-trioxa-3,16,18,24,26-pentaazanonacosanan-1-oyl]-L-prolyl-N—[(R*)—{[15,19-difluoro-3,4-dihydro-2H,11H-10,6-(azeno)-12,16-(metheno)-1,5,11,13-benzodioxadiazacyclooctadecin-8-yl]methyl}(methyl)oxo-lambda6-sulfanylidene]-L-valinamide (Compound 9)

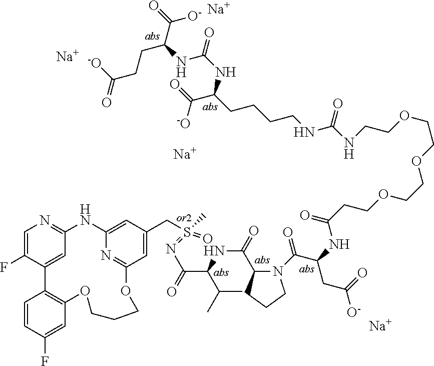

Step 1: To a solution of N-(3-{2-[2-(2-aminoethoxy)ethoxy]ethoxy}propanoyl)-L-alpha-aspartyl-L-prolyl-N—[(R*)—{[15,19-difluoro-3,4-dihydro-2H,11H-10,6-(azeno)-12,16-(metheno)-1,5,11,13-benzodioxadiazacyclooctadecin-8-yl]methyl}(methyl)oxo-lambda⁶-sulfanylidene]-L-valinamide.trifluoroacetic acid (1/1) (27.0 mg, 90% purity, 22.6 μmol) (Intermediate 27) in DMF (5 ml), were added (di-tert-butyl N-{[(2S)-1-tert-butoxy-6-{[(4-nitrophenoxy)carbonyl]amino}-1-oxohexan-2-yl]carbamoyl}-L-glutamate (17.7 mg, 27.1 μmol, CAS Nr: 1610413-98-3, as described in WO2014078484) and DIEA (12 μl, 68 μmol). The mixture was stirred at rt for 5 h and then concentrated under reduced pressure. The residue was purified over preparative HPLC to afford (7S,11S,32S)-7,11-bis(tert-butoxycarbonyl)-32-[(2S)-2-{[(2S)-1-{[(R*)—{[5,22-difluoro-8,12-dioxa-18,20,24-triazatetracyclo[17.3.1.1^{13,17}.0^{2,7}]tetracosa-1(23),2,4,6,13(24),14,16,19,21-nonaen-15-yl]methyl}(methyl)oxo-lambda⁶-sulfanylidene]amino}-3-methyl-1-oxobutan-2-yl]carbamoyl}pyrrolidine-1-carbonyl]-2,2-dimethyl-4,9,17,30-tetraoxo-3,21,24,27-tetraoxa-8,10,16,18,31-pentaazatetratriacontan-34-oic acid (33 mg, 98% purity, 97% yield) as a yellow foam. LC-MS (Method 3): Rₜ=5.53 min; MS (ESIpos): m/z=1474 [M+H]⁺

Step 2: To a solution of (7S,11S,32S)-7,11-bis(tert-butoxycarbonyl)-32-[(2S)-2-{[(2S)-1-{[(R*)—([5,22-difluoro-8,12-dioxa-18,20,24-triazatetracyclo[17.3.1.1^{13,17}.0^{2,7}]tetracosa-1(23),2,4,6,13(24),14,16,19,21-nonaen-15-yl]methyl}(methyl)oxo-lambda⁶-sulfanylidene]amino)-3-methyl-1-oxobutan-2-yl]carbamoyl}pyrrolidine-1-carbonyl]-2,2-dimethyl-4,9,17,30-tetraoxo-3,21,24,27-tetraoxa-8,10,16,18,31-pentaazatetratriacontan-34-oic acid (33.0 mg, 22.4 μmol) in DCM (5.6 ml), was added TFA (1.1 ml). The reaction mixture was stirred for 1 h and concentrated in vacuo. The residue was dissolved in ACN/H₂O and lyophilized to give N-[(3S,7S)-1,3,7-tricarboxy-5,13,26-trioxo-17,20,23-trioxa-4,6,12,14-tetraazahexacosan-26-yl]-L-alpha-aspartyl-L-prolyl-N—[(R*)—{[15,19-difluoro-3,4-dihydro-2H,11H-10,6-(azeno)-12,16-(metheno)-1,5,11,13-benzodioxadiazacyclooctadecin-8-yl]methyl}(methyl)oxo-lambda6-sulfanylidene]-L-valinamide (29 mg, 94% purity, 93% yield) as a colorless foam. LC-MS (Method 3): Rₜ=3.95 min; MS (ESIpos): m/z=1306 [M+H]⁺.

Step 3: To a solution of N-[(3S,7S)-1,3,7-tricarboxy-5,13,26-trioxo-17,20,23-trioxa-4,6,12,14-tetraazahexacosan-26-yl]-L-alpha-aspartyl-L-prolyl-N—[(R*)-{[15,19-difluoro-3,4-dihydro-2H,11H-10,6-(azeno)-12,16-(metheno)-1,5,11,13-benzodioxadiazacyclooctadecin-8-yl]methyl}(methyl)oxo-lambda6-sulfanylidene]-L-valinamide (29.0 mg, 22.2 μmol) in dioxane/water (1:1, 10 mL) was added a sodium hydroxide solution (89 μl, 1 M, 89 μmol). The solution was freeze-dried to give Compound 9 (32 mg, 93% purity, 96% yield) as a colorless foam. LC-MS (Method 3): Rₜ=3.93 min; MS (ESIpos): m/z=1302 [M+H]⁺. ¹H-NMR (400 MHz, DMSO-d6) delta [ppm]: 0.803 (2.74), 0.809 (3.02), 0.820 (3.11), 0.826 (2.88), 0.860 (0.41), 0.875 (0.24), 1.003 (0.17), 1.020 (0.19), 1.132 (0.24), 1.149 (0.29), 1.159 (0.19), 1.166 (0.21), 1.173 (0.23), 1.192 (0.32), 1.210 (0.41), 1.215 (0.46), 1.234 (0.94), 1.254 (0.62), 1.280 (0.92), 1.287 (0.86), 1.323 (0.28), 1.328 (0.25), 1.375 (0.15), 1.409 (0.15), 1.436 (0.23), 1.440 (0.26), 1.450 (0.33), 1.463 (0.33), 1.469 (0.36), 1.471 (0.35), 1.482 (0.31), 1.492 (0.28), 1.505 (0.21), 1.513 (0.19), 1.525 (0.18), 1.538 (0.27), 1.556 (0.47), 1.568 (0.55), 1.580 (0.55), 1.582 (0.54), 1.585 (0.54), 1.598 (0.43), 1.607 (0.35), 1.629 (0.16), 1.748 (0.14), 1.756 (0.16), 1.762 (0.21), 1.766 (0.21), 1.786 (0.30), 1.796 (0.37), 1.807 (0.38), 1.819 (0.40), 1.825 (0.41), 1.829 (0.40), 1.840 (0.51), 1.859 (0.66), 1.872 (0.61), 1.878 (0.65), 1.890 (0.46), 1.894 (0.46), 1.944 (0.78), 1.956 (0.70), 2.001 (0.15), 2.006 (0.14), 2.010 (0.15), 2.020 (0.13), 2.025 (0.16), 2.038 (0.17), 2.043 (0.20), 2.062 (0.43), 2.093 (1.16), 2.109 (1.25), 2.126 (1.11), 2.142 (0.57), 2.160 (0.29), 2.174 (0.20), 2.178 (0.21), 2.215 (0.33), 2.233 (0.66), 2.248 (0.70), 2.258 (0.69), 2.274 (0.83), 2.294 (0.82), 2.310 (0.44), 2.327 (0.48), 2.331 (0.62), 2.336 (0.61), 2.339 (0.52), 2.355 (0.58), 2.370 (0.65), 2.391 (0.38), 2.408 (0.30), 2.431 (0.23), 2.437 (0.26), 2.447 (0.24), 2.453 (0.29), 2.468 (0.36), 2.576 (0.35), 2.599 (0.16), 2.618 (0.13), 2.669 (0.36), 2.673 (0.47), 2.705 (0.13), 2.714 (0.33), 2.736 (0.13), 2.757 (0.16), 2.760 (0.16), 2.763 (0.16), 2.769 (0.18), 2.781 (0.19), 2.787 (0.17), 2.794 (0.17), 2.822 (0.19), 2.826 (0.19), 2.840 (0.20), 2.846 (0.22), 2.861 (0.25), 2.868 (0.26), 2.916 (1.02), 2.927 (1.01), 2.952 (0.40), 2.961 (0.38), 2.967 (0.38), 2.977 (0.36), 2.986 (0.36), 2.995 (0.39), 3.002 (0.38), 3.008 (0.37), 3.019 (0.40), 3.025 (0.40), 3.037 (0.44), 3.049 (0.47), 3.055 (0.49), 3.075 (0.61), 3.106 (1.34), 3.117 (1.44), 3.124 (1.33), 3.140 (1.03), 3.152 (1.06), 3.160 (0.84), 3.169 (1.13), 3.177 (0.95), 3.210 (5.69), 3.228 (1.48), 3.259 (1.74), 3.360 (8.65), 3.392 (16.00), 3.520 (5.89), 3.525 (5.59), 3.550 (4.99), 3.556 (4.40), 3.569 (4.50), 3.582 (3.65), 3.589 (3.76), 3.597 (3.12), 3.602 (5.42), 3.609 (4.85), 3.633 (1.81), 3.640 (1.73), 3.658 (0.84), 3.674 (0.77), 3.696 (0.66), 3.702 (0.63), 3.707 (0.62), 3.714 (0.63), 3.720 (0.62), 3.736 (0.77), 3.746 (0.64), 3.759 (0.79), 3.776 (0.72), 3.788 (0.78), 3.800 (1.08), 3.810 (1.06), 3.815 (0.99), 3.821 (0.99), 3.830 (0.89), 3.844 (0.61), 3.871 (0.25), 3.890 (0.22), 3.898 (0.21), 3.902 (0.23), 3.933 (0.42), 3.946 (0.55), 3.950 (0.55), 3.963 (0.89), 3.976 (0.77), 3.982 (0.72), 3.994 (1.02), 4.003 (0.88), 4.012 (0.77), 4.024 (1.05), 4.040 (0.56), 4.068 (0.16), 4.076 (0.15), 4.095 (0.33), 4.112 (0.87), 4.119 (1.03), 4.155 (0.15), 4.161 (0.14), 4.167 (0.13), 4.240 (0.16), 4.252 (0.17), 4.264 (0.15), 4.324 (0.19), 4.330 (0.74), 4.415 (0.24), 4.426 (0.28), 4.438 (0.29), 4.450 (0.48), 4.465 (0.75), 4.476 (0.55), 4.490 (0.56), 4.502 (0.52), 4.512 (0.67), 4.531 (0.49), 4.636 (0.22), 4.641 (0.24), 4.652 (0.24), 4.658 (0.23), 4.754 (0.21), 4.773 (0.54), 4.793 (0.74), 4.833 (1.45), 4.839 (2.13), 4.845 (1.72), 4.880 (0.27), 4.892 (0.15), 4.908 (0.54), 6.126 (0.15), 6.161 (0.60), 6.166 (0.61), 6.183 (0.62), 6.201 (0.52), 6.208 (0.42), 6.213 (0.41), 6.220 (0.41), 6.225 (0.46), 6.236 (0.41), 6.256 (0.26), 6.271 (0.18), 6.304 (1.87), 6.656 (1.54), 6.872 (0.32), 6.878 (0.36), 6.893 (0.57), 6.899 (0.63), 6.914 (0.29), 6.919 (0.33), 7.056 (0.53), 7.061 (0.53), 7.084 (0.52), 7.090 (0.49), 7.557 (0.29), 7.566 (0.32), 7.575 (0.39), 7.578 (0.45), 7.584 (0.42), 7.596 (0.30), 7.605 (0.24), 7.813 (0.19), 7.837 (0.19), 7.975 (0.50), 7.997 (0.46), 8.234 (0.54), 8.248 (0.56), 8.340 (1.23), 8.346 (1.15), 8.490 (0.33), 8.680 (0.99), 8.695 (0.94), 9.927 (0.99).

### Example 10: Preparation of sodium 1-{(2S)-2-(carboxylatomethyl)-4,17,20-trioxo-20-[(5-sulfamoyl-1,3,4-thiadiazol-2-yl)amino]-7,10,13-trioxa-3,16-diazaicosanan-1-oyl}-L-prolyl-N—[(R*)—{[15,19-difluoro-3,4-dihydro-2H,11H-10,6-(azeno)-12,16-(metheno)-1,5,11,13-benzodioxadiazacyclooctadecin-8-yl]methyl}(methyl)oxo-lambda6-sulfanylidene]-L-valinamide (Compound 10)

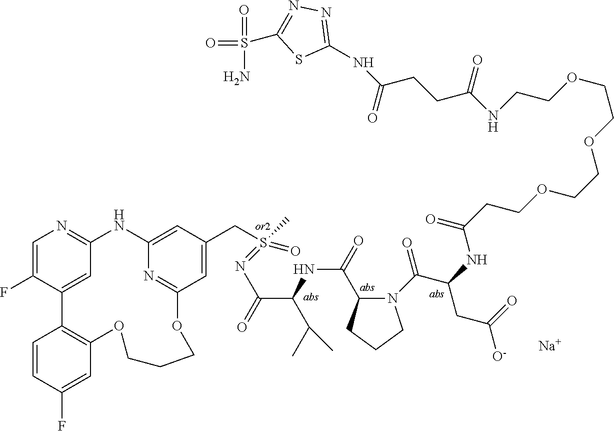 [SMILES: CC(C)[C@H](NC(=O)[C@@H]1CCCN1C(=O)[C@H](CC(=O)[O-])NC(=O)CCOCCOCCOCCNC(=O)CCC(=O)Nc1nnc(S(N)(=O)=O)s1)C(=O)N=[S@](C)(=O)Cc1cc2nc(c1)OCCCOc1cc(F)ccc1-c1cc(ncc1F)N2 ; [Na+]]

Step 1: To a solution of N-(3-{2-[2-(2-aminoethoxy)ethoxy]ethoxy}propanoyl)-L-alpha-aspartyl-L-prolyl-N—[(R*)—{[15,19-difluoro-3,4-dihydro-2H,11H-10,6-(azeno)-12,16-(metheno)-1,5,11,13-benzodioxadiazacyclooctadecin-8-yl]methyl}(methyl)oxo-lambda⁶-sulfanylidene]-L-valinamide.trifluoroacetic acid (1/1) (25.0 mg, 90% purity, 20.9 μmol) (Intermediate 27) in DMF (5.9 ml), were added pentafluorophenyl 4-oxo-4-[(5-sulfamoyl-1,3,4-thiadiazol-2-yl)amino]butanoate (99.6 mg, 75% purity, 167 μmol) (Intermediate 19) and DIEA (29 μl, 170 μmol). The mixture was stirred at rt for 1 h and then concentrated under reduced pressure. The residue was purified over preparative HPLC and freeze dried to afford N-{14,17-dioxo-17-[(5-sulfamoyl-1,3,4-thiadiazol-2-yl)amino]-4,7,10-trioxa-13-azaheptadecanan-1-oyl}-L-alpha-aspartyl-L-prolyl-N—[(R*)-[{15,19-difluoro-3,4-dihydro-2H,11H-10,6-(azeno)-12,16-(metheno)-1,5,11,13-benzodioxadiazacyclooctadecin-8-yl]methyl}(methyl)oxo-lambda6-sulfanylidene]-L-valinamide (18 mg, 87% purity, 61% yield) LC-MS (Method 3): Rₜ=4.09 min; MS (ESIpos): m/z=1223 [M+H]⁺.

Step 2: To a solution of N-{14,17-dioxo-17-[(5-sulfamoyl-1,3,4-thiadiazol-2-yl)amino]-4,7,10-trioxa-13-azaheptadecanan-1-oyl}-L-alpha-aspartyl-L-prolyl-N—[(R*)—{[15,19-difluoro-3,4-dihydro-2H,11H-10,6-(azeno)-12,16-(metheno)-1,5,11,13-benzodioxadiazacyclooctadecin-8-yl]methyl}(methyl)oxo-lambda6-sulfanylidene]-L-valinamide (18.0 mg, 14.7 μmol) in dioxane/water (1:1, 5 mL) was added a sodium hydroxide solution (150 μl, 0.1 M, 15 μmol). The solution was freeze-dried to give Compound 10 (18.4 mg, 90% purity, 91% yield) as a colorless foam. LC-LC-MS (Method 3): Rₜ=4.09 min; MS (ESIpos): m/z=1222 [M+H]⁺. 1H-NMR (400 MHz, DMSO-d6) delta [ppm]: 0.819 (4.05), 0.835 (4.44), 0.851 (4.39), 0.868 (4.33), 0.902 (0.42), 1.235 (0.80), 1.826 (0.24), 1.840 (0.40), 1.854 (0.67), 1.866 (0.83), 1.876 (0.84), 1.895 (0.71), 1.915 (0.69), 1.928 (0.57), 1.958 (1.80), 1.972 (1.29), 2.081 (1.59), 2.097 (1.84), 2.113 (1.52), 2.127 (0.96), 2.144 (0.48), 2.157 (0.32), 2.297 (0.29), 2.318 (1.28), 2.333 (2.64), 2.350 (1.33), 2.380 (0.71), 2.401 (0.77), 2.422 (0.98), 2.442 (0.98), 2.457 (1.45), 2.474 (2.99), 2.612 (0.23), 2.615 (0.23), 2.632 (0.24), 2.649 (0.24), 2.684 (2.30), 2.701 (2.88), 2.720 (1.84), 2.735 (0.79), 2.751 (0.25), 2.850 (0.24), 2.858 (0.24), 2.871 (0.25), 2.875 (0.27), 2.896 (0.27), 2.903 (0.26), 2.912 (0.28), 2.915 (0.28), 2.918 (0.29), 2.936 (0.30), 2.939 (0.32), 2.945 (0.33), 2.950 (0.37), 2.963 (0.33), 2.972 (0.37), 2.990 (0.39), 2.999 (0.42), 3.005 (0.45), 3.013 (0.46), 3.021 (0.47), 3.028 (0.48), 3.038 (0.50), 3.041 (0.51), 3.044 (0.50), 3.050 (0.53), 3.067 (0.60), 3.073 (0.62), 3.082 (0.64), 3.099 (0.75), 3.105 (0.77), 3.110 (0.80), 3.129 (0.93), 3.163 (2.68), 3.186 (10.51), 3.206 (2.86), 3.219 (2.18), 3.236 (2.43), 3.374 (15.20), 3.468 (11.51), 3.491 (16.00), 3.521 (3.33), 3.557 (4.51), 3.569 (6.77), 3.571 (7.01), 3.590 (4.08), 3.603 (4.42), 3.610 (4.21), 3.634 (2.09), 3.640 (1.94), 3.653 (1.75), 3.674 (1.67), 3.691 (1.34), 3.748 (0.52), 3.758 (0.49), 3.769 (0.45), 3.779 (0.45), 3.781 (0.45), 3.793 (0.40), 3.799 (0.40), 3.802 (0.40), 3.806 (0.39), 3.812 (0.38), 3.821 (0.40), 3.833 (0.35), 3.840 (0.34), 3.848 (0.32), 3.863 (0.29), 3.870 (0.31), 3.877 (0.31), 3.889 (0.27), 3.918 (0.27), 3.948 (0.27), 3.965 (0.62), 3.980 (0.66), 3.983 (0.68), 3.996 (0.99), 4.013 (0.66), 4.025 (0.51), 4.044 (0.24), 4.058 (1.01), 4.072 (1.10), 4.079 (1.15), 4.093 (1.11), 4.124 (2.12), 4.164 (0.24), 4.456 (0.28), 4.499 (1.61), 4.511 (2.11), 4.524 (1.74), 4.747 (1.08), 4.781 (1.47), 4.841 (1.69), 4.861 (1.71), 4.877 (1.05), 4.894 (1.66), 4.912 (0.49), 6.318 (3.43), 6.587 (2.88), 6.640 (0.24), 6.880 (0.60), 6.902 (1.19), 6.924 (0.68), 7.065 (1.08), 7.089 (1.08), 7.093 (1.04), 7.559 (0.45), 7.568 (0.58), 7.579 (0.83), 7.584 (0.83), 7.597 (0.62), 7.607 (0.53), 7.777 (1.15), 7.799 (1.15), 7.980 (0.77), 7.993 (1.43), 8.006 (0.77), 8.179 (0.23), 8.186 (0.25), 8.192 (0.27), 8.212 (0.33), 8.221 (0.30), 8.233 (0.32), 8.247 (0.32), 8.256 (0.28), 8.263 (0.29), 8.286 (0.45), 8.303 (1.37), 8.327 (2.86), 8.333 (2.52), 8.676 (0.24), 8.689 (1.76), 8.703 (1.67), 9.653 (2.40), 11.994 (0.13), 12.005 (0.16), 12.014 (0.18), 12.019 (0.18), 12.027 (0.19), 12.045 (0.21), 12.049 (0.19), 12.067 (0.15), 12.086 (0.12), 12.092 (0.12), 12.095 (0.12).

### Example 11: Preparation of tetrasodium (1S,9S)-1-amino-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl 1-[(2S,23S,27S)-23,27,29-tricarboxylato-2-(carboxylatomethyl)-4,17,25-trioxo-7,10,13-trioxa-3,16,18,24,26-pentaazanonacosanan-1-oyl]-L-prolyl-L-valinate (Compound 11)

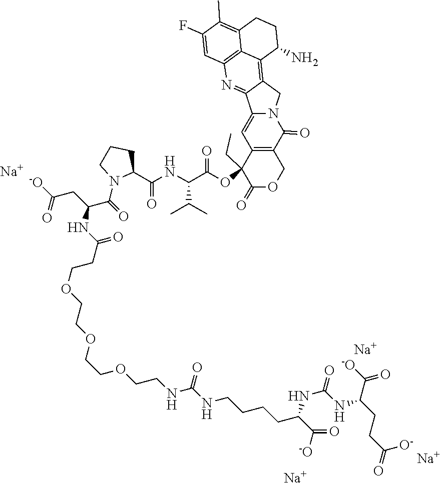

Step 1: To a solution of (1S,9S)-1-amino-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-(3-{2-[2-(2-aminoethoxy)ethoxy]ethoxy}propanoyl)-L-alpha-aspartyl-L-prolyl-L-valinate.trifluoroacetic acid (1/1) (40.0 mg, 100% purity, 37.6 μmol) (Intermediate 33) in DMF (8 ml), were added (di-tert-butyl N-{[(2S)-1-tert-butoxy-6-{[(4-nitrophenoxy)carbonyl]amino}-1-oxohexan-2-yl]carbamoyl}-L-glutamate (27.0 mg, 41.4 μmol, CAS Nr: 1610413-98-3, as described in WO2014078484) and DIEA (19.6 μl, 113 μmol). The mixture was stirred at rt for 16 h and then concentrated under reduced pressure. The residue was purified over preparative HPLC and freeze dried to afford (7S,11S,32S)-32-[(2S)-2-{[(2S)-1-{[(1S,9S)-1-amino-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl]oxy}-3-methyl-1-oxobutan-2-yl]carbamoyl}pyrrolidine-1-carbonyl]-7,11-bis(tert-butoxycarbonyl)-2,2-dimethyl-4,9,17,30-tetraoxo-3,21,24,27-tetraoxa-8,10,16,18,31-pentaazatetratriacontan-34-oic acid (24.1 mg, 100% purity, 44% yield). LC-MS (Method 3): Rₜ=3.96 min; MS (ESIpos): m/z=1464 [M+H]⁺

Step 2: To a solution of (7S,11S,32S)-32-[(2S)-2-{[(2S)-1-{[(1S,9S)-1-amino-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl]oxy}-3-methyl-1-oxobutan-2-yl]carbamoyl}pyrrolidine-1-carbonyl]-7,11-bis(tert-butoxycarbonyl)-2,2-dimethyl-4,9,17,30-tetraoxo-3,21,24,27-tetraoxa-8,10,16,18,31-pentaazatetratriacontan-34-oic acid (29.1 mg, 19.9 μmol) in DCM (3 ml), was added TFA (3 ml). The reaction mixture was stirred for 2 h and concentrated in vacuo. The residue was purified over preparative HPLC and lyophilized to give (1S,9S)-1-amino-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-[(3S,7S)-1,3,7-tricarboxy-5,13,26-trioxo-17,20,23-trioxa-4,6,12,14-tetraazahexacosan-26-yl]-L-alpha-aspartyl-L-prolyl-L-valinate (25.3 mg, 91% purity, 89% yield) as an amorphous residue. LC-MS (Method 3): Rₜ=2.60 min; MS (ESIpos): m/z=1296 [M+H]⁺.

Step 3: To a solution of (1S,9S)-1-amino-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-[(3S,7S)-1,3,7-tricarboxy-5,13,26-trioxo-17,20,23-trioxa-4,6,12,14-tetraazahexacosan-26-yl]-L-alpha-aspartyl-L-prolyl-L-valinate (25.1 mg, 19.4 μmol) in dioxane/water (1:1, 8 mL) was added a sodium hydroxide solution (78 μl, 1 M, 78 μmol). The solution was freeze-dried to give Compound 11(25.9 mg, 100% purity, 97% yield) as a colorless foam. LC-MS (Method 3): Rₜ=2.56 min; MS (ESIpos): m/z=1296 [M+H]⁺. ¹H-NMR (500 MHz, D₂O) δ [ppm]: 0.932 (2.26), 0.947 (3.89), 0.962 (2.03), 0.969 (1.13), 0.997 (3.74), 1.011 (3.54), 1.024 (3.47), 1.038 (3.34), 1.261 (0.81), 1.274 (1.24), 1.290 (1.00), 1.385 (0.83), 1.398 (0.97), 1.407 (0.87), 1.872 (0.67), 1.936 (0.83), 1.952 (0.84), 2.000 (1.74), 2.161 (1.93), 2.178 (2.70), 2.194 (1.66), 2.235 (4.33), 2.255 (1.29), 2.270 (1.56), 2.386 (0.75), 2.399 (0.70), 2.470 (0.76), 2.483 (0.81), 2.502 (0.75), 2.531 (0.95), 2.543 (1.71), 2.555 (0.85), 2.741 (0.67), 2.990 (1.34), 3.004 (2.40), 3.017 (1.31), 3.027 (0.70), 3.060 (0.78), 3.187 (1.27), 3.198 (2.48), 3.209 (1.38), 3.390 (0.90), 3.401 (0.86), 3.414 (0.98), 3.425 (1.19), 3.437 (0.66), 3.480 (1.85), 3.491 (3.03), 3.502 (1.94), 3.584 (3.68), 3.594 (16.00), 3.603 (6.25), 3.609 (7.97), 3.622 (12.20), 3.639 (1.38), 3.644 (0.66), 3.652 (0.90), 3.656 (0.91), 3.669 (0.86), 3.675 (0.95), 3.685 (4.05), 3.689 (2.37), 3.692 (2.97), 3.695 (3.70), 3.704 (2.90), 3.718 (4.58), 3.725 (0.79), 3.735 (1.34), 3.747 (2.39), 3.755 (1.82), 3.759 (2.15), 3.778 (1.27), 3.783 (1.43), 3.918 (13.08), 3.939 (2.10), 3.945 (1.66), 4.369 (1.64), 4.382 (1.27), 5.514 (0.96), 5.547 (1.27), 5.666 (1.20), 5.700 (0.83), 7.321 (2.47), 7.434 (0.93), 7.455 (0.78), 8.412 (4.99).

### Example 12: Preparation of sodium (1S,9S)-1-amino-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl 1-{(2S)-2-(carboxylatomethyl)-4,17,20-trioxo-20-[(5-sulfamoyl-1,3,4-thiadiazol-2-yl)amino]-7,10,13-trioxa-3,16-diazaicosanan-1-oyl}-L-prolyl-L-valinate (Compound 12)

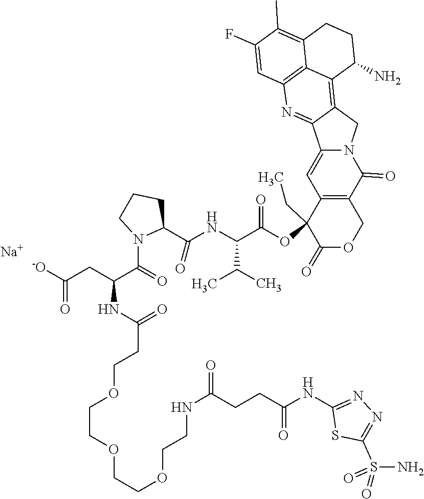

Step 1: To a solution of (1S,9S)-1-amino-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-(3-{2-[2-(2-aminoethoxy)ethoxy]ethoxy}propanoyl)-L-alpha-aspartyl-L-prolyl-L-valinate.trifluoroacetic acid (1/1) (40.0 mg, 100% purity, 37.6 μmol) (Intermediate 33) in DMF (10 ml), were added pentafluorophenyl 4-oxo-4-[(5-sulfamoyl-1,3,4-thiadiazol-2-yl)amino]butanoate (101 mg, 75% purity, 169 μmol) (Intermediate 19) and DIEA (29 μl, 170 μmol). The mixture was stirred at rt for 1 h and then concentrated under reduced pressure. The residue was purified over preparative HPLC and freeze dried to afford (1S,9S)-1-amino-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-{14,17-dioxo-17-[(5-sulfamoyl-1,3,4-thiadiazol-2-yl)amino]-4,7,10-trioxa-13-azaheptadecanan-1-oyl}-L-alpha-aspartyl-L-prolyl-L-valinate (12.2 mg, 100% purity, 27% yield) as an amorphous residue. LC-MS (Method 2): Rₜ=1.05 min; MS (ESIneg): m/z=1210 [M−H]⁻

Step 2: To a solution of (1S,9S)-1-amino-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-{14,17-dioxo-17-[(5-sulfamoyl-1,3,4-thiadiazol-2-yl)amino]-4,7,10-trioxa-13-azaheptadecanan-1-oyl}-L-alpha-aspartyl-L-prolyl-L-valinate (12.0 mg, 9.90 μmol) in dioxane/water (1:1, 5 mL) was added a sodium hydroxide solution (9.9 μl, 1 M, 9.9 μmol). The solution was freeze-dried to give Compound 12 (12 mg, 100% purity, 98% yield) as a colorless foam. LC-MS (Method 3): Rₜ=2.59 min; MS (ESIpos): m/z=1212 [M+H]⁺. ¹H-NMR (400 MHz, DMSO-d6) δ[ppm]: 0.821 (1.34), 0.841 (2.16), 0.860 (4.32), 0.875 (5.73), 0.888 (7.71), 0.902 (10.43), 0.916 (6.13), 0.984 (1.12), 1.000 (1.20), 1.829 (1.15), 1.848 (1.30), 1.857 (1.19), 1.863 (1.16), 1.873 (1.31), 1.877 (1.32), 1.880 (1.26), 1.889 (1.64), 1.904 (1.25), 1.931 (1.17), 1.939 (1.11), 1.957 (1.13), 1.965 (1.30), 1.973 (1.28), 2.028 (1.12), 2.031 (1.12), 2.041 (1.09), 2.049 (1.09), 2.056 (1.13), 2.074 (1.08), 2.168 (1.11), 2.198 (2.78), 2.207 (2.75), 2.215 (2.88), 2.223 (2.68), 2.225 (2.64), 2.244 (2.20), 2.260 (2.35), 2.283 (2.41), 2.299 (3.00), 2.317 (3.52), 2.322 (3.27), 2.327 (3.03), 2.336 (3.04), 2.361 (7.22), 2.371 (7.66), 2.386 (5.74), 2.406 (3.46), 2.566 (1.23), 2.660 (1.63), 2.665 (1.85), 2.669 (1.77), 2.694 (2.61), 2.705 (1.55), 3.106 (1.90), 3.116 (1.79), 3.133 (2.93), 3.150 (4.70), 3.164 (5.31), 3.179 (4.02), 3.200 (3.42), 3.560 (16.00), 3.579 (6.90), 3.594 (5.66), 3.600 (5.54), 3.749 (1.29), 3.754 (1.35), 3.757 (1.36), 3.764 (1.40), 3.770 (1.48), 3.800 (3.10), 3.813 (3.17), 3.816 (3.19), 3.836 (2.16), 3.886 (1.66), 3.906 (2.37), 3.927 (1.49), 4.241 (1.10), 4.322 (1.69), 4.406 (1.13), 4.610 (1.14), 4.613 (1.11), 4.810 (1.36), 4.829 (1.44), 4.950 (1.13), 4.958 (1.12), 4.970 (1.48), 5.344 (2.24), 5.396 (1.52), 5.445 (3.54), 5.458 (3.64), 7.408 (2.21), 7.414 (2.51), 7.487 (1.33), 7.504 (1.28), 7.512 (1.46), 7.530 (1.16), 7.959 (1.37), 7.971 (2.23), 7.984 (1.46), 8.217 (1.51), 8.234 (1.81), 8.399 (1.16), 8.555 (1.14), 8.568 (1.13).

### Example 13: disodium (1S,2R)-2-({(2R,3R)-3-methoxy-3-[(2S)-1-((3R,4S,5S)-3-methoxy-5-methyl-4-[methyl(N-methyl-L-valyl-L-valyl)amino]heptanoyl)pyrrolidin-2-yl]-2-methylpropanoyl}amino)-1-phenylpropyl 1-[(2S)-2-(carboxylatomethyl)-17-{4-[({(1R)-2-carboxylato-1-[3-({3-[(propylcarbamoyl)amino]benzene-1-sulfonyl}amino)phenyl]ethyl}carbamoyl)amino]anilino}-4,17-dioxo-7,10,13-trioxa-3,16-diazaheptadecanan-1-oyl]-L-prolyl-L-valinate (Compound 13)

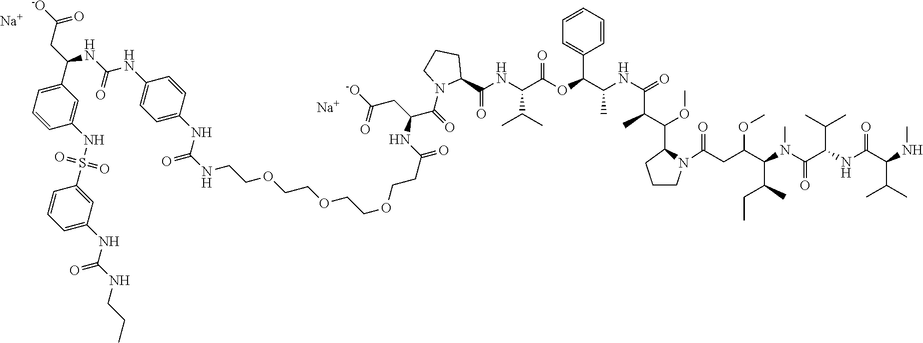

Step 1: To a solution of (1S,2R)-2-({(2R,3R)-3-[(2S)-1-{(3R,4S,5S)-4-[{N-[(benzyloxy)carbonyl]-N-methyl-L-valyl-L-valyl}(methyl)amino]-3-methoxy-5-methylheptanoyl}pyrrolidin-2-yl]-3-methoxy-2-methylpropanoyl}amino)-1-phenylpropyl N-(3-{2-[2-(2-aminoethoxy)ethoxy]ethoxy}propanoyl)-L-alpha-aspartyl-L-prolyl-L-valinate.trifluoroacetic acid (1/1) (246 mg, 93% purity, 154 μmol) (Intermediate 35) in DMF (40 ml), were added (3R)-3-{[(4-{[(4-nitrophenoxy)carbonyl]amino}phenyl)carbamoyl]amino}-3-[3-({3-[(propylcarbamoyl)amino]benzene-1-sulfonyl}amino)phenyl]propanoic acid (175 mg, 95% purity, 231 μmol) (Intermediate 2) and DIEA (130 μl, 770 μmol). The mixture was stirred at rt for 1 h and then concentrated under reduced pressure. The residue was purified over preparative HPLC and freeze dried to afford (1S,2R)-2-({(2R,3R)-3-[(2S)-1-{(3R,4S,5S)-4-[{N-[(benzyloxy)carbonyl]-N-methyl-L-valyl-L-valyl}(methyl)amino]-3-methoxy-5-methylheptanoyl)pyrrolidin-2-yl]-3-methoxy-2-methylpropanoyl}amino)-1-phenylpropyl N-(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl}-L-alpha-aspartyl-L-prolyl-L-valinate (241 mg, 97% purity, 78% yield) as an amorphous residue. LC-MS (Method 3): Rₜ=5.15 min; MS (ESIpos): m/z=1947 [M+H]⁺.

Step 2: (1S,2R)-2-({(2R,3R)-3-[(2S)-1-{(3R,4S,5S)-4-[{N-[(benzyloxy)carbonyl]-N-methyl-L-valyl-L-valyl}(methyl)amino]-3-methoxy-5-methylheptanoyl)pyrrolidin-2-yl]-3-methoxy-2-methylpropanoyl}amino)-1-phenylpropyl N-(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl}-L-alpha-aspartyl-L-prolyl-L-valinate (189 mg, 97.3 μmol) was dissolved in ethanol (30 ml). Pd/C 10% (18.9 mg) was added and the reaction was hydrogenated at RT for 4h 30 and filtered. The mother liquor was concentrated in vacuo, purified over preparative HPLC and lyophilized to give (1S,2R)-2-({(2R,3R)-3-methoxy-3-[(2S)-1-{(3R,4S,5S)-3-methoxy-5-methyl-4-[methyl(N-methyl-L-valyl-L-valyl)amino]heptanoyl)pyrrolidin-2-yl]-2-methylpropanoyl}amino)-1-phenylpropyl N-(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl}-L-alpha-aspartyl-L-prolyl-L-valinate (116 mg, 94% purity, 61% yield) as an amorphous residue. LC-MS (Method 3): Rₜ=3.49 min; MS (ESIpos): m/z=1813 [M+H]⁺.

Step 3: To a solution of (1S,2R)-2-({(2R,3R)-3-methoxy-3-[(2S)-1-((3R,4S,5S)-3-methoxy-5-methyl-4-[methyl(N-methyl-L-valyl-L-valyl)amino]heptanoyl)pyrrolidin-2-yl]-2-methylpropanoyl)amino)-1-phenylpropyl N-(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl}-L-alpha-aspartyl-L-prolyl-L-valinate (25.0 mg, 94% purity, 12.9 μmol) in dioxane/water (1:1, 14 mL) was added a sodium hydroxide solution (26 μl, 1 M, 26 μmol). The solution was freeze-dried to give Compound 13 (23.9 mg, 96% purity, 96% yield) as a colorless foam. LC-MS (Method 3): Rₜ=3.49 min; MS (ESIpos): m/z=1813 [M+H]⁺. ¹H-NMR (400 MHz, DMSO-d6) delta [ppm]: −0.148 (0.05), −0.034 (0.04), 0.146 (0.04), 0.655 (0.04), 0.664 (0.05), 0.682 (0.23), 0.698 (0.26), 0.710 (0.26), 0.718 (0.15), 0.727 (0.27), 0.756 (0.37), 0.776 (0.40), 0.794 (0.24), 0.811 (0.55), 0.822 (0.53), 0.830 (1.23), 0.847 (1.28), 0.861 (0.87), 0.872 (0.71), 0.886 (0.91), 0.898 (0.75), 0.925 (0.12), 0.937 (0.09), 0.943 (0.09), 0.962 (0.08), 0.984 (0.09), 0.992 (0.09), 0.996 (0.08), 1.017 (0.24), 1.034 (0.29), 1.043 (0.35), 1.060 (0.34), 1.074 (0.27), 1.091 (0.22), 1.223 (0.04), 1.238 (0.12), 1.262 (0.06), 1.265 (0.06), 1.272 (0.06), 1.285 (0.07), 1.292 (0.07), 1.298 (0.07), 1.308 (0.07), 1.320 (0.09), 1.338 (0.18), 1.357 (0.30), 1.374 (0.29), 1.393 (0.16), 1.405 (0.05), 1.410 (0.06), 1.418 (0.05), 1.424 (0.05), 1.446 (0.06), 1.451 (0.06), 1.476 (0.08), 1.479 (0.07), 1.487 (0.07), 1.503 (0.08), 1.507 (0.08), 1.511 (0.07), 1.519 (0.07), 1.527 (0.06), 1.537 (0.06), 1.550 (0.05), 1.556 (0.05), 1.566 (0.05), 1.579 (0.04), 1.668 (0.06), 1.686 (0.09), 1.693 (0.08), 1.700 (0.11), 1.717 (0.11), 1.727 (0.12), 1.729 (0.11), 1.743 (0.12), 1.747 (0.11), 1.758 (0.12), 1.766 (0.11), 1.773 (0.11), 1.782 (0.12), 1.791 (0.13), 1.797 (0.13), 1.799 (0.13), 1.812 (0.13), 1.820 (0.14), 1.824 (0.14), 1.835 (0.16), 1.847 (0.14), 1.857 (0.16), 1.863 (0.16), 1.869 (0.16), 1.878 (0.15), 1.883 (0.15), 1.897 (0.13), 1.911 (0.09), 1.919 (0.09), 1.930 (0.09), 1.948 (0.11), 1.957 (0.10), 1.964 (0.12), 1.985 (0.11), 2.004 (0.08), 2.011 (0.08), 2.032 (0.06), 2.040 (0.06), 2.044 (0.05), 2.056 (0.05), 2.071 (0.04), 2.076 (0.04), 2.083 (0.06), 2.097 (0.05), 2.104 (0.04), 2.111 (0.07), 2.123 (0.09), 2.144 (0.57), 2.156 (0.11), 2.160 (0.12), 2.174 (0.57), 2.191 (0.14), 2.214 (0.14), 2.231 (0.21), 2.246 (0.16), 2.271 (0.13), 2.277 (0.11), 2.289 (0.11), 2.306 (0.07), 2.331 (0.13), 2.352 (0.08), 2.369 (0.14), 2.389 (0.12), 2.406 (0.20), 2.409 (0.20), 2.416 (0.20), 2.440 (0.12), 2.447 (0.12), 2.457 (0.12), 2.466 (0.14), 2.503 (16.00), 2.577 (0.14), 2.580 (0.14), 2.586 (0.14), 2.617 (0.11), 2.626 (0.09), 2.645 (0.12), 2.662 (0.13), 2.675 (0.20), 2.692 (0.09), 2.713 (0.08), 2.811 (0.05), 2.870 (0.04), 2.879 (0.04), 2.889 (0.05), 2.905 (0.13), 2.921 (0.27), 2.937 (0.26), 2.953 (0.16), 2.974 (0.36), 3.004 (0.06), 3.011 (0.06), 3.022 (0.08), 3.025 (0.08), 3.041 (0.08), 3.050 (0.08), 3.061 (0.08), 3.068 (0.10), 3.078 (0.09), 3.084 (0.09), 3.090 (0.09), 3.106 (0.10), 3.124 (0.18), 3.142 (0.29), 3.151 (0.49), 3.178 (0.70), 3.196 (0.35), 3.212 (0.82), 3.221 (0.84), 3.238 (0.43), 3.256 (0.89), 3.426 (1.03), 3.437 (0.74), 3.468 (0.53), 3.478 (0.45), 3.482 (0.45), 3.500 (0.73), 3.519 (1.17), 3.544 (0.41), 3.551 (0.36), 3.568 (8.52), 3.594 (0.23), 3.601 (0.24), 3.615 (0.27), 3.640 (0.09), 3.647 (0.09), 3.661 (0.07), 3.672 (0.06), 3.678 (0.07), 3.690 (0.07), 3.696 (0.07), 3.704 (0.07), 3.711 (0.06), 3.743 (0.06), 3.754 (0.08), 3.757 (0.08), 3.775 (0.12), 3.781 (0.13), 3.789 (0.14), 3.800 (0.20), 3.818 (0.18), 3.831 (0.16), 3.836 (0.13), 3.933 (0.08), 3.952 (0.14), 3.972 (0.10), 3.983 (0.07), 3.991 (0.07), 3.995 (0.08), 4.004 (0.08), 4.012 (0.08), 4.017 (0.08), 4.022 (0.07), 4.034 (0.04), 4.042 (0.04), 4.094 (0.04), 4.107 (0.04), 4.117 (0.04), 4.137 (0.06), 4.153 (0.06), 4.158 (0.06), 4.164 (0.06), 4.173 (0.07), 4.179 (0.06), 4.189 (0.07), 4.196 (0.07), 4.203 (0.07), 4.213 (0.06), 4.219 (0.07), 4.230 (0.05), 4.237 (0.06), 4.248 (0.04), 4.331 (0.15), 4.381 (0.10), 4.414 (0.06), 4.427 (0.07), 4.439 (0.04), 4.554 (0.06), 4.575 (0.08), 4.597 (0.06), 4.610 (0.06), 4.635 (0.15), 4.650 (0.12), 4.655 (0.13), 4.665 (0.06), 4.713 (0.07), 4.728 (0.12), 4.743 (0.12), 4.944 (0.06), 4.967 (0.10), 5.577 (0.09), 5.592 (0.08), 5.634 (0.08), 5.644 (0.06), 5.649 (0.07), 6.615 (0.04), 6.620 (0.04), 6.642 (0.16), 6.658 (0.15), 6.752 (0.04), 6.792 (0.06), 6.797 (0.05), 6.808 (0.05), 6.812 (0.04), 6.930 (0.14), 6.947 (0.18), 7.041 (0.16), 7.061 (0.23), 7.080 (0.10), 7.168 (0.16), 7.187 (0.24), 7.197 (0.09), 7.215 (0.20), 7.240 (0.62), 7.246 (0.71), 7.272 (0.37), 7.283 (0.68), 7.291 (0.57), 7.311 (0.25), 7.324 (0.12), 7.329 (0.12), 7.347 (0.04), 7.515 (0.24), 7.758 (0.05), 7.769 (0.04), 7.778 (0.05), 7.854 (0.23), 7.856 (0.23), 7.984 (0.11), 8.004 (0.12), 8.008 (0.14), 8.034 (0.07), 8.098 (0.07), 8.110 (0.09), 8.113 (0.10), 8.128 (0.07), 8.163 (0.14), 8.184 (0.14), 8.236 (0.06), 8.545 (0.06), 8.551 (0.06), 8.556 (0.06), 8.562 (0.06), 8.578 (0.05), 8.585 (0.04), 8.729 (0.10), 8.744 (0.08), 8.747 (0.07), 8.809 (0.19), 9.111 (0.03), 9.122 (0.04), 9.130 (0.04), 9.140 (0.05), 9.144 (0.05), 9.149 (0.04), 11.210 (0.15).

### Example 14: Preparation of trisodium (1S,2R)-2-({(2R,3R)-3-methoxy-3-[(2S)-1-{(3R,4S,5S)-3-methoxy-5-methyl-4-[methyl(N-methyl-L-valyl-L-valyl)amino]heptanoyl)pyrrolidin-2-yl]-2-methylpropanoyl}amino)-1-phenylpropyl 1-[(2S)-15{[N2,N6-bis(14-{4-[({(1R)-2-carboxylato-1-[3-((3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl}-L-lysyl]amino)-2-(carboxylatomethyl)-4-oxo-7,10,13-trioxa-3-azapentadecanan-1-oyl]-L-prolyl-L-valinate (Compound 14)

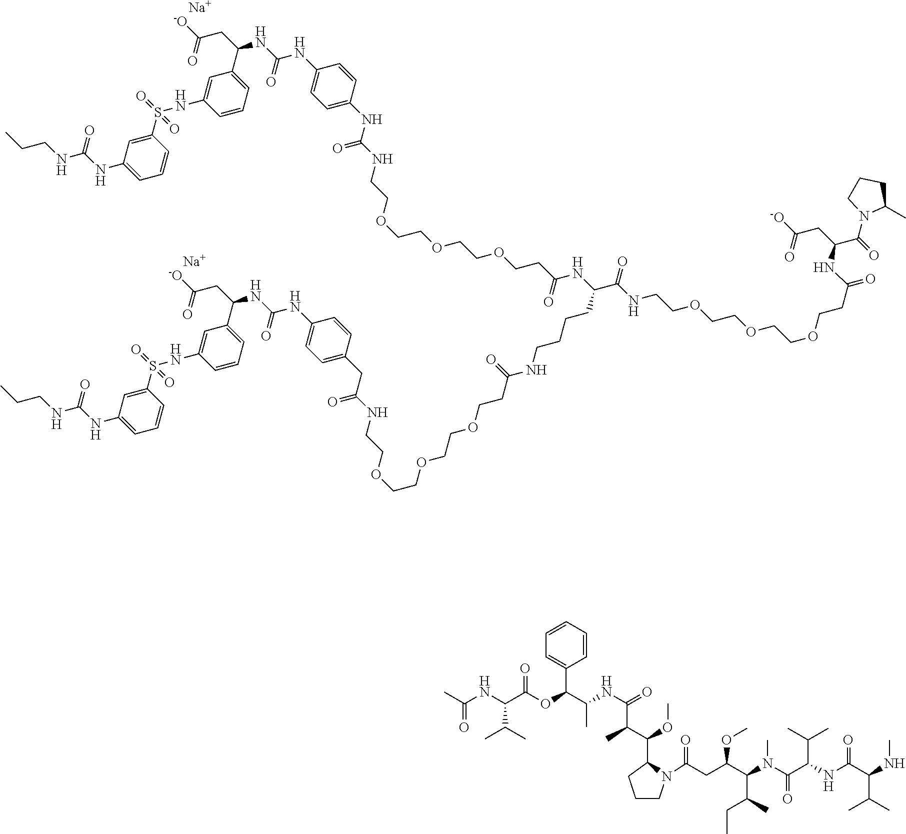

Step 1: To a solution of (1S,2R)-2-({(2R,3R)-3-[(2S)-1-((3R,4S,5S)-4-[{(N-[(benzyloxy)carbonyl]-N-methyl-L-valyl-L-valyl}(methyl)amino]-3-methoxy-5-methylheptanoyl)pyrrolidin-2-yl]-3-methoxy-2-methylpropanoyl}amino)-1-phenylpropyl N-(3-{2-[2-(2-{[N²,N⁶-bis(3-(2-[2-(2-aminoethoxy)ethoxy]ethoxy}propanoyl)-L-lysyl]amino)ethoxy)ethoxy]ethoxy}propanoyl)-L-alpha-aspartyl-L-prolyl-L-valinate.trifluoroacetic acid (1/2) (204 mg, 100% purity, 95.9 μmol) (Intermediate 37) in DMF (20 ml), were added (3R)-3-{[(4-{[(4-nitrophenoxy)carbonyl]amino}phenyl)carbamoyl]amino}-3-[3-({3-[(propylcarbamoyl)amino]benzene-1-sulfonyl}amino)phenyl]propanoic acid (138 mg, 192 μmol) (Intermediate 2) and DIEA (330 μl, 1.9 mmol). The mixture was stirred at rt for 2 h and then concentrated under reduced pressure. The residue was purified over preparative HPLC and freeze dried to afford (1S,2R)-2-({(2R,3R)-3-[(2S)-1-{(3R,4S,5S)-4-[(N-[(benzyloxy)carbonyl]-N-methyl-L-valyl-L-valyl}(methyl)amino]-3-methoxy-5-methylheptanoyl)pyrrolidin-2-yl]-3-methoxy-2-methylpropanoyl)amino)-1-phenylpropyl N-(3-{2-[2-(2-{[N²,N⁶-bis(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl}-L-lysyl]amino}ethoxy)ethoxy]ethoxy}propanoyl)-L-alpha-aspartyl-L-prolyl-L-valinate (244 mg, 100% purity, 83% yield) as an amorphous residue. LC-MS (Method 3): Rₜ=5.07 min; MS (ESIpos): m/z=1531 [M+2H]2+

Step 2: (1S,2R)-2-({(2R,3R)-3-[(2S)-1-{(3R,4S,5S)-4-[(N-[(benzyloxy)carbonyl]-N-methyl-L-valyl-L-valyl}(methyl)amino]-3-methoxy-5-methylheptanoyl)pyrrolidin-2-yl]-3-methoxy-2-methylpropanoyl}amino)-1-phenylpropyl N-(3-{2-[2-(2-{[N²,N⁶-bis(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl}-L-lysyl]amino}ethoxy)ethoxy]ethoxy}propanoyl)-L-alpha-aspartyl-L-prolyl-L-valinate (244 mg, 79.7 μmol) was dissolved in ethanol (30 ml). Pd/C 10% (24.4 mg) was added, and the reaction was hydrogenated at RT for 20 h and filtered over celite. The mother liquor was concentrated in vacuo, purified over preparative HPLC and lyophilized to give (1S,2R)-2-({(2R,3R)-3-methoxy-3-[(2S)-1-{(3R,4S,5S)-3-methoxy-5-methyl-4-[methyl(N-methyl-L-valyl-L-valyl)amino]heptanoyl)pyrrolidin-2-yl]-2-methylpropanoyl}amino)-1-phenylpropyl N-{3-{2-[2-(2-([N2,N6-bis(14-{4-[({(1R)-2-carboxy-1-[3-((3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl}-L-lysyl]amino)ethoxy)ethoxy]ethoxy}propanoyl)-L-alpha-aspartyl-L-prolyl-L-valinate (123 mg, 100% purity, 53% yield) as an amorphous residue. LC-MS (Method 3): Rₜ=3.68 min; MS (ESIpos): m/z=1464 [M+2H]²⁺.

Step 3: To a solution of (1S,2R)2-2-({(2R,3R)-3-methoxy-3-[(2S)-1-{(3R,4S,5S)-3-methoxy-5-methyl-4-[methyl(N-methyl-L-valyl-L-valyl)amino]heptanoyl)pyrrolidin-2-yl]-2-methylpropanoyl)amino)-1-phenylpropyl N-(3-{2-[2-{2-([N2,N6-bis(14-{4-[({(1R)-2-carboxy-1-[3-((3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl}-L-lysyl]amino}ethoxy)ethoxy]ethoxy}propanoyl)-L-alpha-aspartyl-L-prolyl-L-valinate (25.0 mg, 100% purity, 8.54 μmol) in dioxane/water (1:1, 8 mL) was added a sodium hydroxide solution (26 μl, 1 M, 26 μmol). The solution was freeze-dried to give Compound 14 (24.8 mg, 100% purity, 97% yield) as a colorless foam. LC-MS (Method 3): Rₜ=3.68 min; MS (ESIpos): m/z=1464 [M(free acid)+2H]²⁺. ¹H-NMR (400 MHz, DMSO-d6) delta [ppm]: 0.662 (1.01), 0.678 (1.09), 0.702 (1.06), 0.718 (1.06), 0.754 (1.83), 0.775 (1.98), 0.809 (5.38), 0.828 (11.36), 0.846 (7.70), 0.858 (3.63), 0.872 (3.44), 0.885 (5.02), 0.899 (3.96), 1.015 (1.31), 1.041 (1.78), 1.058 (1.81), 1.072 (1.31), 1.089 (1.06), 1.235 (1.02), 1.318 (0.98), 1.336 (2.30), 1.354 (3.61), 1.372 (3.22), 1.390 (1.65), 1.473 (0.72), 1.889 (0.94), 1.966 (0.61), 2.142 (3.22), 2.172 (3.10), 2.222 (0.88), 2.270 (1.53), 2.286 (2.28), 2.302 (1.23), 2.329 (0.85), 2.367 (1.47), 2.378 (1.97), 2.394 (1.31), 2.583 (1.26), 2.615 (0.80), 2.641 (0.69), 2.670 (1.22), 2.711 (0.43), 2.903 (1.10), 2.919 (2.31), 2.935 (2.37), 2.951 (1.22), 2.974 (2.40), 3.123 (1.20), 3.140 (1.66), 3.151 (2.88), 3.168 (2.48), 3.178 (4.12), 3.210 (5.89), 3.220 (5.07), 3.255 (4.50), 3.402 (5.76), 3.409 (5.75), 3.424 (6.06), 3.438 (3.92), 3.482 (11.78), 3.514 (16.00), 3.568 (9.32), 3.591 (4.13), 3.600 (2.43), 3.800 (0.79), 3.829 (0.68), 3.899 (0.59), 4.004 (0.40), 4.194 (0.72), 4.329 (0.62), 4.373 (0.49), 4.633 (0.79), 4.653 (0.75), 4.727 (0.50), 4.969 (0.90), 5.565 (0.18), 5.573 (0.36), 5.582 (0.22), 5.588 (0.38), 5.635 (0.32), 5.649 (0.33), 6.457 (0.52), 6.631 (1.42), 6.650 (1.41), 6.930 (1.34), 6.948 (1.70), 7.038 (1.57), 7.057 (2.39), 7.076 (1.03), 7.166 (1.53), 7.186 (2.37), 7.197 (2.06), 7.220 (4.36), 7.240 (5.06), 7.263 (2.38), 7.274 (2.78), 7.284 (3.33), 7.294 (3.24), 7.314 (1.38), 7.358 (0.97), 7.378 (0.90), 7.507 (2.50), 7.837 (0.81), 7.864 (2.29), 7.980 (0.87), 8.003 (0.70), 8.029 (0.36), 8.040 (0.29), 8.050 (0.57), 8.070 (0.59), 8.112 (0.48), 8.120 (0.35), 8.179 (1.43), 8.200 (1.31), 8.457 (0.58), 8.561 (0.35), 8.664 (0.58), 8.673 (0.57), 8.695 (0.58), 8.758 (1.05), 8.858 (1.78), 11.235 (1.69).

### Example 15: Preparation of N2-(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl}-L-asparaginyl-L-prolyl-N-{3-[{(1R)-1-[1-benzyl-4-(2,5-difluorophenyl)-1H-pyrrol-2-yl]-2,2-dimethylpropyl}(hydroxyacetyl)amino]propyl}-L-alaninamide (Compound 15)

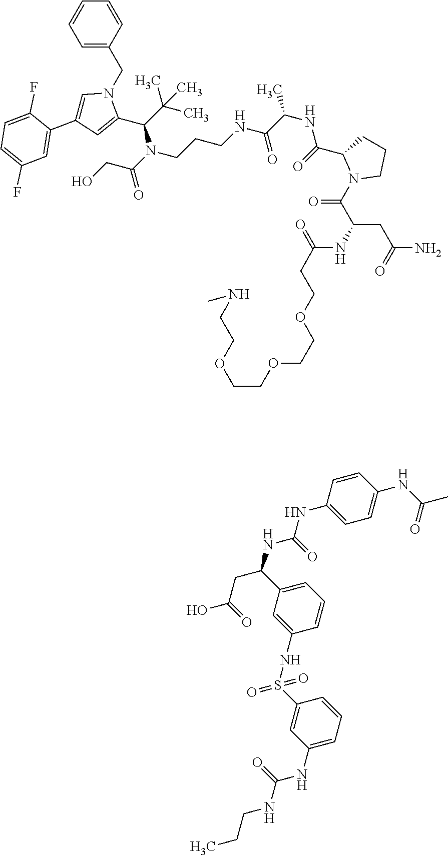 [SMILES: CNCCOCCOCCOCCC(=O)N[C@@H](CC(N)=O)C(=O)N1CCC[C@H]1C(=O)N[C@@H](C)C(=O)NCCCN(C(=O)CO)[C@@H](c1cc(-c2cc(F)ccc2F)cn1Cc1ccccc1)C(C)(C)C ; CCCNC(=O)Nc1cccc(S(=O)(=O)Nc2cccc([C@@H](CC(=O)O)NC(=O)Nc3ccc(NC(C)=O)cc3)c2)c1]

To a solution of trifluoroacetic acid. N²-(3-{2-[2-(2-aminoethoxy)ethoxy]ethoxy}propanoyl)-L-asparaginyl-L-prolyl-N-{3-[{(1R)-1-[1-benzyl-4-(2,5-difluorophenyl)-1H-pyrrol-2-yl]-2,2-dimethylpropyl}(hydroxyacetyl)amino]propyl}-L-alaninamide (1/1) (15.0 mg, 14.0 μmol) (Intermediate 40) in DMF (7 ml), were added (3R)-3-{[(4-{[(4-nitrophenoxy)carbonyl]amino}phenyl)carbamoyl]amino}-3-[3-({3-[(propylcarbamoyl)amino]benzene-1-sulfonyl}amino)phenyl]propanoic acid (10.1 mg, 14.0 μmol) (Intermediate 2) and DIEA (24 μl, 140 μmol). The mixture was stirred at rt for 30 min and then concentrated under reduced pressure. The residue was purified over preparative HPLC and freeze dried to afford Compound 15 (15 mg, 100% purity, 70% yield) as a colorless foam. LC-MS (Method 3): Rₜ=4.58 min; MS (ESIpos): m/z=1535 [M+H]⁺. ¹H-NMR (600 MHz, DMSO-d6) delta [ppm]: 0.386 (0.32), 0.395 (0.31), 0.825 (16.00), 0.843 (3.33), 0.856 (6.65), 0.868 (3.36), 1.160 (0.31), 1.177 (3.50), 1.189 (3.47), 1.235 (0.43), 1.244 (0.38), 1.256 (0.34), 1.259 (0.31), 1.264 (0.26), 1.380 (0.65), 1.398 (0.28), 1.410 (1.13), 1.422 (2.09), 1.434 (2.05), 1.446 (1.06), 1.839 (1.56), 1.845 (1.57), 1.855 (0.80), 1.866 (0.30), 2.037 (0.26), 2.047 (0.38), 2.051 (0.49), 2.058 (0.38), 2.066 (0.33), 2.310 (0.26), 2.324 (0.58), 2.335 (1.47), 2.345 (1.51), 2.356 (0.67), 2.362 (0.58), 2.371 (0.71), 2.387 (0.86), 2.397 (0.56), 2.517 (0.63), 2.520 (0.61), 2.523 (0.52), 2.575 (0.58), 2.589 (0.62), 2.600 (0.53), 2.615 (0.80), 2.631 (2.04), 2.643 (1.97), 2.654 (0.45), 2.669 (0.44), 2.680 (0.43), 2.689 (0.28), 2.693 (0.29), 2.704 (0.26), 2.807 (0.38), 2.816 (0.42), 2.828 (0.36), 2.837 (0.29), 3.017 (0.74), 3.027 (1.60), 3.038 (1.60), 3.049 (0.71), 3.122 (0.27), 3.132 (0.32), 3.139 (0.46), 3.146 (0.33), 3.157 (0.34), 3.165 (0.26), 3.215 (1.42), 3.223 (1.46), 3.288 (0.34), 3.293 (0.37), 3.311 (0.55), 3.332 (0.34), 3.337 (0.33), 3.400 (0.27), 3.408 (0.32), 3.420 (1.81), 3.429 (2.99), 3.438 (1.64), 3.461 (2.17), 3.465 (2.16), 3.469 (2.93), 3.482 (3.21), 3.487 (2.56), 3.490 (2.56), 3.496 (1.77), 3.531 (2.03), 3.556 (2.67), 3.561 (3.07), 3.567 (3.24), 3.572 (3.70), 3.577 (3.68), 3.582 (3.04), 3.588 (2.83), 3.622 (1.53), 3.633 (1.53), 3.640 (1.44), 3.649 (1.37), 3.660 (0.97), 3.705 (0.39), 3.725 (0.53), 3.737 (0.77), 3.750 (0.62), 3.753 (0.64), 3.762 (0.36), 3.765 (0.39), 3.994 (1.03), 4.020 (1.20), 4.049 (0.68), 4.062 (0.98), 4.074 (0.64), 4.212 (0.70), 4.217 (0.61), 4.225 (0.68), 4.231 (0.65), 4.255 (1.17), 4.281 (0.97), 4.778 (0.32), 4.792 (0.63), 4.802 (0.64), 4.816 (0.32), 4.882 (0.84), 4.908 (1.01), 4.980 (0.33), 4.991 (0.83), 5.004 (0.80), 5.016 (0.32), 5.156 (1.01), 5.182 (0.88), 5.601 (1.86), 6.064 (0.58), 6.192 (0.52), 6.201 (0.93), 6.211 (0.51), 6.615 (0.98), 6.629 (0.91), 6.898 (0.89), 6.900 (0.89), 6.913 (1.08), 6.923 (1.82), 6.958 (0.45), 6.966 (0.50), 6.972 (0.77), 6.980 (1.37), 6.994 (1.25), 7.026 (1.07), 7.140 (2.03), 7.143 (2.19), 7.156 (1.79), 7.169 (0.85), 7.180 (2.03), 7.192 (2.51), 7.206 (9.82), 7.217 (0.91), 7.220 (0.85), 7.224 (0.82), 7.235 (0.49), 7.242 (1.13), 7.255 (2.04), 7.267 (1.98), 7.280 (2.46), 7.293 (0.81), 7.331 (1.83), 7.343 (2.63), 7.357 (1.57), 7.368 (0.50), 7.398 (1.18), 7.411 (0.87), 7.505 (2.05), 7.635 (0.38), 7.641 (0.44), 7.646 (0.48), 7.651 (0.67), 7.657 (0.46), 7.662 (0.42), 7.668 (0.35), 7.804 (1.02), 7.817 (0.98), 8.054 (2.12), 8.197 (1.00), 8.210 (0.95), 8.326 (1.97), 8.346 (1.91), 8.748 (2.29), 10.268 (2.86), 12.030 (0.02), 12.034 (0.02), 12.052 (0.02), 12.060 (0.03), 12.075 (0.02), 12.081 (0.03), 12.102 (0.03), 12.108 (0.03), 12.113 (0.02), 12.122 (0.03), 12.130 (0.02), 12.145 (0.03), 12.147 (0.02), 12.149 (0.03), 12.157 (0.03), 12.161 (0.03), 12.163 (0.03), 12.171 (0.03), 12.175 (0.03), 12.183 (0.03), 12.188 (0.03), 12.193 (0.03), 12.198 (0.05), 12.199 (0.04), 12.203 (0.03), 12.208 (0.03), 12.216 (0.04), 12.227 (0.04), 12.238 (0.04), 12.244 (0.04), 12.249 (0.04), 12.253 (0.03), 12.256 (0.04), 12.278 (0.04), 12.282 (0.04), 12.284 (0.04), 12.290 (0.04), 12.296 (0.04), 12.302 (0.05), 12.306 (0.04), 12.316 (0.04), 12.333 (0.04), 12.345 (0.04), 12.347 (0.04), 12.351 (0.04), 12.354 (0.04), 12.357 (0.04), 12.359 (0.03), 12.389 (0.04), 12.397 (0.03), 12.400 (0.03), 12.404 (0.03), 12.408 (0.02), 12.411 (0.02), 12.417 (0.02), 12.419 (0.03), 12.422 (0.02), 12.432 (0.03), 12.438 (0.03), 12.444 (0.03), 12.455 (0.03), 12.460 (0.02), 12.467 (0.03), 12.470 (0.03), 12.472 (0.03), 12.501 (0.03), 12.505 (0.02).

### Example 16: Preparation of N²-(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl}-L-asparaginyl-L-prolyl-N-{3-[{(1R)-1-[1-benzyl-4-(2,5-difluorophenyl)-1H-pyrrol-2-yl]-2,2-dimethylpropyl}(hydroxyacetyl)amino]propyl}-L-valinamide (Compound 16)

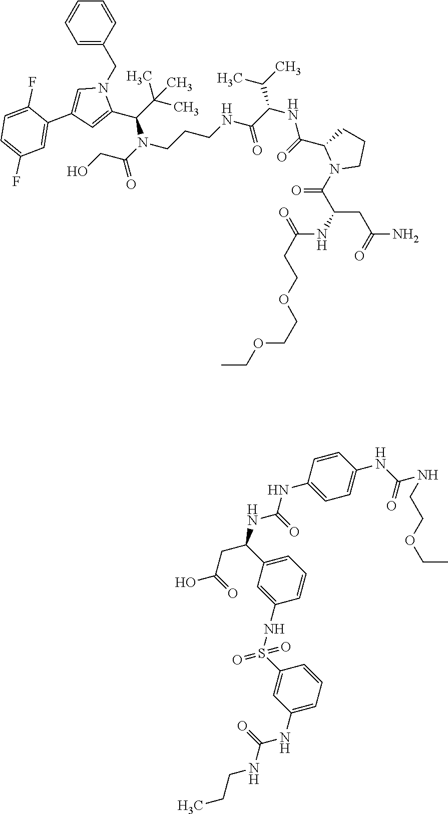 [SMILES: CCOCCOCCC(=O)N[C@@H](CC(N)=O)C(=O)N1CCC[C@H]1C(=O)N[C@H](C(=O)NCCCN(C(=O)CO)[C@@H](c1cc(-c2cc(F)ccc2F)cn1Cc1ccccc1)C(C)(C)C)C(C)C ; CCCNC(=O)Nc1cccc(S(=O)(=O)Nc2cccc([C@@H](CC(=O)O)NC(=O)Nc3ccc(NC(=O)NCCOCC)cc3)c2)c1]

To a solution of trifluoroacetic acid-N2-(3-{2-[2-(2-aminoethoxy)ethoxy]ethoxy}propanoyl)-L-asparaginyl-L-prolyl-N-{3-[{(1R)-1-[1-benzyl-4-(2,5-difluorophenyl)-1H-pyrrol-2-yl]-2,2-dimethylpropyl}(hydroxyacetyl)amino]propyl}-L-valinamide (1/1) (15.0 mg, 13.7 μmol) (Intermediate 42) in DMF (6.8 ml), were added (3R)-3-{[(4-{[(4-nitrophenoxy)carbonyl]amino)phenyl)carbamoyl]amino}-3-[3-((3-[(propylcarbamoyl)amino]benzene-1-sulfonyl}amino)phenyl]propanoic acid (9.84 mg, 13.7 μmol) (Intermediate 2) and DIEA (24 μl, 140 μmol). The mixture was stirred at rt for 30 min and then concentrated under reduced pressure. The residue was purified over preparative HPLC and freeze dried to afford Compound 16 (12 mg, 100% purity, 56% yield) as a colorless foam LC-MS (Method 3): Rₜ=4.74 min; MS (ESIpos): m/z=1563 [M+H]⁺. ¹H-NMR (600 MHz, DMSO-d6) delta [ppm]: 0.067 (0.22), 0.322 (0.26), 0.331 (0.34), 0.340 (0.34), 0.350 (0.26), 0.741 (3.54), 0.753 (3.66), 0.774 (3.65), 0.785 (3.77), 0.800 (0.67), 0.821 (16.00), 0.842 (3.15), 0.854 (6.29), 0.866 (3.14), 0.909 (0.25), 1.201 (0.28), 1.213 (0.35), 1.221 (0.38), 1.232 (0.42), 1.377 (0.40), 1.396 (0.28), 1.409 (1.15), 1.421 (2.09), 1.432 (2.04), 1.444 (1.04), 1.836 (1.26), 1.849 (0.87), 1.859 (0.69), 1.866 (0.67), 1.878 (0.69), 1.889 (0.63), 1.901 (0.37), 1.956 (0.29), 1.969 (0.40), 1.980 (0.42), 1.989 (0.30), 2.299 (0.32), 2.310 (0.87), 2.323 (1.74), 2.334 (2.12), 2.345 (1.11), 2.358 (0.32), 2.384 (0.25), 2.515 (0.57), 2.521 (0.85), 2.612 (0.31), 2.629 (2.00), 2.641 (1.92), 2.652 (0.26), 2.691 (0.25), 2.703 (0.38), 2.714 (0.43), 2.726 (0.34), 2.779 (0.40), 2.788 (0.47), 2.800 (0.35), 2.811 (0.25), 3.015 (0.77), 3.026 (1.64), 3.036 (1.60), 3.047 (0.70), 3.120 (0.24), 3.129 (0.28), 3.146 (0.50), 3.164 (0.35), 3.172 (0.29), 3.214 (1.45), 3.222 (1.47), 3.285 (0.42), 3.304 (0.61), 3.324 (0.40), 3.330 (0.39), 3.347 (0.30), 3.349 (0.30), 3.363 (0.35), 3.375 (0.36), 3.419 (2.10), 3.428 (3.33), 3.438 (2.11), 3.458 (3.03), 3.461 (2.96), 3.466 (3.77), 3.482 (4.53), 3.487 (4.17), 3.490 (4.46), 3.498 (3.99), 3.551 (3.96), 3.562 (3.99), 3.572 (2.61), 3.590 (1.40), 3.598 (1.30), 3.607 (1.28), 3.648 (0.48), 3.655 (0.61), 3.667 (0.84), 3.680 (0.69), 3.889 (0.68), 3.902 (0.94), 3.916 (0.65), 4.001 (0.99), 4.027 (1.13), 4.249 (1.17), 4.275 (0.96), 4.333 (0.75), 4.345 (0.69), 4.350 (0.69), 4.781 (0.31), 4.793 (0.86), 4.805 (0.86), 4.817 (0.31), 4.882 (0.85), 4.908 (1.05), 4.978 (0.33), 4.990 (0.83), 5.003 (0.82), 5.014 (0.33), 5.153 (0.97), 5.179 (0.86), 5.601 (1.96), 6.064 (0.56), 6.191 (0.52), 6.200 (0.92), 6.209 (0.52), 6.614 (0.95), 6.627 (0.90), 6.898 (0.96), 6.917 (2.13), 6.928 (1.18), 6.947 (0.28), 6.953 (0.45), 6.967 (0.75), 6.980 (1.48), 6.993 (1.23), 7.138 (2.08), 7.141 (2.21), 7.155 (1.74), 7.168 (0.83), 7.181 (2.09), 7.194 (2.95), 7.204 (9.65), 7.220 (0.98), 7.229 (0.50), 7.241 (0.86), 7.254 (1.96), 7.265 (1.74), 7.269 (1.47), 7.278 (2.17), 7.291 (0.79), 7.330 (1.82), 7.342 (2.46), 7.355 (1.00), 7.382 (0.23), 7.399 (1.88), 7.409 (1.15), 7.495 (1.52), 7.559 (0.50), 7.568 (0.94), 7.578 (0.52), 7.622 (0.41), 7.627 (0.49), 7.632 (0.53), 7.638 (0.78), 7.648 (1.40), 7.662 (0.96), 8.052 (2.08), 8.161 (0.99), 8.174 (0.95), 8.327 (1.93), 8.345 (1.87), 8.748 (2.29), 10.267 (2.77), 12.106 (0.03), 12.109 (0.04), 12.180 (0.05), 12.185 (0.05), 12.199 (0.04), 12.209 (0.04), 12.211 (0.04), 12.216 (0.05), 12.219 (0.05), 12.222 (0.05), 12.227 (0.04), 12.241 (0.05), 12.251 (0.04), 12.254 (0.04), 12.259 (0.05), 12.266 (0.04), 12.271 (0.03), 12.281 (0.04), 12.284 (0.05), 12.289 (0.05), 12.295 (0.04), 12.304 (0.04), 12.314 (0.05), 12.316 (0.05), 12.322 (0.05), 12.331 (0.05), 12.333 (0.04), 12.346 (0.04), 12.349 (0.04), 12.358 (0.04), 12.373 (0.03), 12.378 (0.04), 12.412 (0.04), 12.441 (0.03).

### Example 17: Preparation of sodium N²-(14-{4-[({(1R)-2-carboxylato-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)-L-asparaginyl-L-prolyl-N-{3-[{(1R)-1-[1-benzyl-4-(2,5-difluorophenyl)-1H-pyrrol-2-yl]-2,2-dimethylpropyl}(hydroxyacetyl)amino]propyl}-L-valinamide (Compound 17)

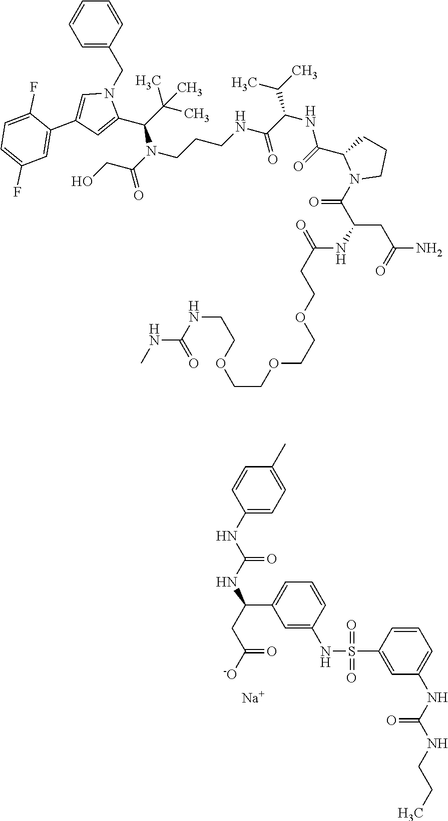

To a solution of N²-(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)-L-asparaginyl-L-prolyl-N-{3-[{(1R)-1-[1-benzyl-4-(2,5-difluorophenyl)-1H-pyrrol-2-yl]-2,2-dimethylpropyl}(hydroxyacetyl)amino]propyl}-L-valinamide (100 mg, 93% purity, 59.6 μmol) (Compound 16) in dioxane/water (1:1, 20 mL) was added a sodium hydroxide solution (600 μl, 0.1 M, 60 μmol). The solution was freeze-dried to give Compound 17 (100 mg, 95% purity, quant.) as a colorless foam. LC-MS (Method 3): Rₜ=4.72 min; MS (ESIpos): m/z=1563 [M+H]⁺. ¹H-NMR (600 MHz, DMSO-d6) delta [ppm]: 0.409 (0.14), 0.421 (0.14), 0.442 (0.14), 0.453 (0.15), 0.489 (0.63), 0.510 (0.13), 0.522 (0.25), 0.534 (0.12), 0.856 (0.00), 0.859 (0.01), 0.869 (0.01), 0.880 (0.01), 0.884 (0.01), 0.889 (0.01), 0.894 (0.01), 0.900 (0.01), 1.076 (0.04), 1.088 (0.08), 1.100 (0.07), 1.112 (0.04), 1.504 (0.04), 1.617 (0.01), 1.625 (0.01), 1.637 (0.01), 1.648 (0.02), 1.656 (0.01), 1.661 (0.01), 1.991 (0.06), 2.002 (0.08), 2.013 (0.05), 2.053 (0.03), 2.166 (3.41), 2.169 (4.57), 2.172 (3.32), 2.209 (3.87), 2.295 (0.07), 2.307 (0.06), 2.360 (0.01), 2.370 (0.01), 2.382 (0.02), 2.393 (0.01), 2.437 (0.01), 2.447 (0.01), 2.456 (0.02), 2.466 (0.01), 2.479 (0.01), 2.691 (0.06), 2.702 (0.06), 2.796 (0.01), 2.815 (0.02), 2.821 (0.01), 2.833 (0.01), 2.835 (0.01), 2.841 (0.01), 2.881 (0.07), 2.890 (0.07), 2.992 (16.00), 3.087 (0.06), 3.096 (0.10), 3.106 (0.05), 3.126 (0.07), 3.134 (0.09), 3.150 (0.09), 3.159 (0.07), 3.186 (0.51), 3.235 (0.15), 3.557 (0.02), 3.571 (0.03), 3.583 (0.02), 3.664 (0.02), 3.673 (0.02), 3.690 (0.02), 3.700 (0.02), 3.912 (0.02), 3.922 (0.02), 3.939 (0.02), 3.948 (0.02), 4.000 (0.03), 4.013 (0.03), 4.018 (0.03), 4.240 (0.03), 4.249 (0.06), 4.259 (0.02), 4.449 (0.01), 4.453 (0.01), 4.461 (0.03), 4.473 (0.03), 4.485 (0.01), 4.550 (0.03), 4.576 (0.04), 4.644 (0.01), 4.657 (0.03), 4.670 (0.03), 4.681 (0.01), 4.814 (0.01), 4.821 (0.04), 4.848 (0.03), 5.269 (0.08), 5.725 (0.02), 5.734 (0.04), 5.743 (0.02), 5.861 (0.01), 5.871 (0.01), 5.881 (0.01), 5.887 (0.01), 6.294 (0.01), 6.305 (0.01), 6.317 (0.01), 6.585 (0.08), 6.646 (0.05), 6.659 (0.05), 6.807 (0.05), 6.821 (0.07), 6.834 (0.03), 6.849 (0.08), 6.862 (0.11), 6.873 (0.42), 6.907 (0.04), 6.920 (0.06), 6.933 (0.06), 6.938 (0.05), 6.947 (0.08), 6.959 (0.03), 6.999 (0.07), 7.011 (0.10), 7.024 (0.04), 7.069 (0.05), 7.164 (0.06), 7.237 (0.04), 7.317 (0.05), 7.331 (0.04), 7.715 (0.03), 7.830 (0.04), 7.842 (0.04), 7.997 (0.08), 8.023 (0.03), 9.929 (0.04).

### Example 18: Preparation of disodium N²-(3-{2-[2-(2-{[N²,N⁶-bis(14-{4-[({(1R)-2-carboxylato-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)-L-lysyl]amino}ethoxy)ethoxy]ethoxy}propanoyl)-L-asparaginyl-L-prolyl-N-{3-[{(1R)-1-[1-benzyl-4-(2,5-difluorophenyl)-1H-pyrrol-2-yl]-2,2-dimethylpropyl}(hydroxyacetyl)amino]propyl}-L-valinamide (Compound 18)

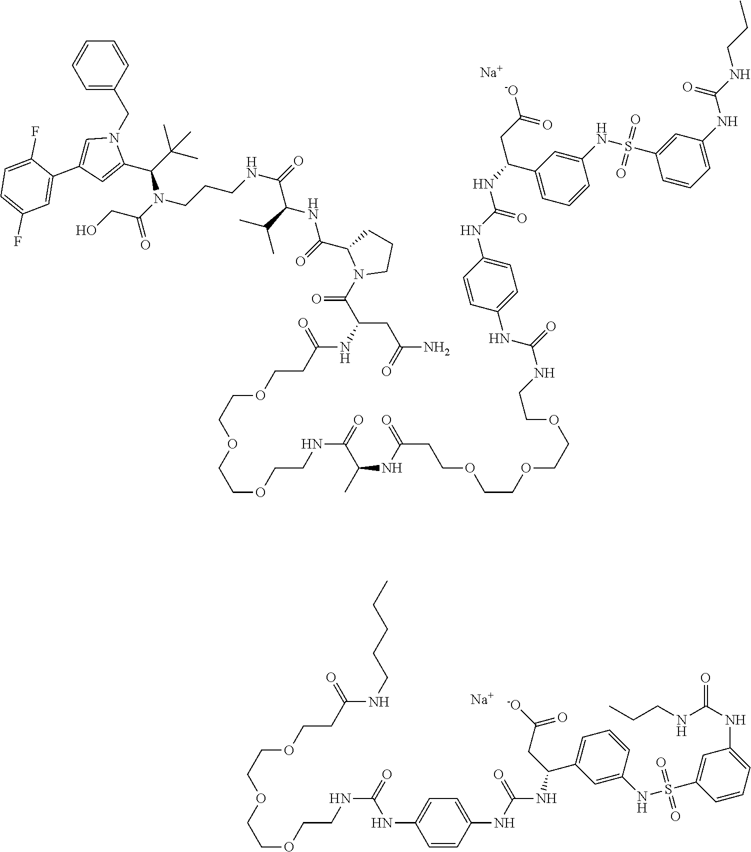

Step 1: To a solution of trifluoroacetic acid. N²-(3-{2-[2-(2-{[N²,N⁶-bis(3-{2-[2-(2-aminoethoxy)ethoxy]ethoxy}propanoyl)-L-lysyl]amino}ethoxy)ethoxy]ethoxy}propanoyl)-L-asparaginyl-L-prolyl-N-{3-[{(1R)-1-[1-benzyl-4-(2,5-difluorophenyl)-1H-pyrrol-2-yl]-2,2-dimethylpropyl}(hydroxyacetyl)amino]propyl}-L-valinamide (2/1) (187 mg, 107 μmol) (Intermediate 44) in DMF (20 ml), were added (3R)-3-{[(4-{[(4-nitrophenoxy)carbonyl]amino}phenyl)carbamoyl]amino}-3-[3-({3-[(propylcarbamoyl)amino]benzene-1-sulfonyl}amino)phenyl]propanoic acid (178 mg, 95% purity, 236 μmol) (Intermediate 2) and DIEA (190 μl, 1.1 mmol). The mixture was stirred at rt for 15 min and then concentrated under reduced pressure. The residue was purified over preparative HPLC and freeze dried to afford N²-(3-{2-[2-(2-{[N²,N⁶-bis(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)-L-lysyl]amino}ethoxy)ethoxy]ethoxy}propanoyl)-L-asparaginyl-L-prolyl-N-{3-[{(1R)-1-[1-benzyl-4-(2,5-difluorophenyl)-1H-pyrrol-2-yl]-2,2-dimethylpropyl}(hydroxyacetyl)amino]propyl}-L-valinamide (155 mg, 90% purity, 49% yield) as a white amorphous residue. LC-MS (Method 3): Rₜ=4.85 min; MS (ESIpos): m/z=2677 [M+H]⁺.

Step 2: To a solution of N²-(3-{2-[2-(2-{[N²,N⁶-bis(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)-L-lysyl]amino}ethoxy)ethoxy]ethoxy}propanoyl)-L-asparaginyl-L-prolyl-N-{3-[{(1R)-1-[1-benzyl-4-(2,5-difluorophenyl)-1H-pyrrol-2-yl]-2,2-dimethylpropyl}(hydroxyacetyl)amino]propyl}-L-valinamide (155 mg, 90% purity, 52.2 μmol) in dioxane/water (1:1, 30 mL) was added a sodium hydroxide solution (100 μl, 1 M, 100 μmol). The solution was freeze-dried to give Compound 18 (155 mg, 91% purity, 99% yield) as an amorphous residue. LC-MS (Method 3): Rₜ=4.83 min; MS (ESIpos): m/z=2677 [M+H]⁺1H-NMR (400 MHz, DMSO-d6) delta [ppm]: 0.317 (0.27), 0.327 (0.32), 0.339 (0.32), 0.358 (0.23), 0.741 (2.83), 0.757 (3.02), 0.774 (3.06), 0.790 (3.25), 0.822 (13.21), 0.836 (12.57), 0.855 (5.90), 0.871 (0.32), 0.906 (0.27), 0.909 (0.27), 1.189 (0.55), 1.208 (0.78), 1.211 (0.73), 1.217 (0.78), 1.228 (0.87), 1.236 (0.96), 1.265 (0.37), 1.318 (0.37), 1.336 (0.73), 1.344 (0.91), 1.361 (2.24), 1.379 (3.38), 1.397 (3.11), 1.415 (1.74), 1.434 (0.59), 1.444 (0.37), 1.449 (0.37), 1.463 (0.37), 1.469 (0.41), 1.475 (0.32), 1.480 (0.32), 1.565 (0.32), 1.582 (0.37), 1.592 (0.32), 1.601 (0.32), 1.802 (0.37), 1.833 (1.14), 1.842 (1.14), 1.861 (0.91), 1.878 (0.82), 1.895 (0.64), 1.913 (0.37), 1.954 (0.32), 1.962 (0.27), 1.971 (0.37), 1.974 (0.37), 1.999 (0.32), 2.270 (1.10), 2.286 (2.29), 2.303 (1.55), 2.326 (1.60), 2.332 (1.65), 2.341 (1.46), 2.351 (1.19), 2.369 (1.74), 2.384 (1.37), 2.402 (0.64), 2.421 (0.37), 2.437 (0.27), 2.463 (0.27), 2.466 (0.32), 2.562 (0.59), 2.573 (1.42), 2.589 (1.23), 2.593 (1.14), 2.599 (1.37), 2.612 (1.37), 2.640 (0.50), 2.653 (0.46), 2.664 (0.32), 2.669 (0.50), 2.674 (0.59), 2.678 (0.50), 2.693 (0.27), 2.702 (0.37), 2.714 (0.64), 2.732 (0.37), 2.747 (0.27), 2.750 (0.27), 2.758 (0.27), 2.763 (0.27), 2.778 (0.41), 2.791 (0.46), 2.810 (0.32), 2.820 (0.27), 2.825 (0.27), 2.932 (0.27), 2.945 (1.28), 2.962 (2.74), 2.977 (3.15), 2.994 (1.97), 3.011 (0.59), 3.017 (0.59), 3.043 (0.27), 3.055 (0.27), 3.061 (0.27), 3.064 (0.27), 3.072 (0.27), 3.091 (0.27), 3.097 (0.37), 3.100 (0.32), 3.109 (0.41), 3.124 (0.55), 3.141 (0.78), 3.156 (0.82), 3.174 (1.42), 3.190 (1.83), 3.204 (2.10), 3.212 (3.29), 3.226 (3.47), 3.239 (1.97), 3.254 (1.01), 3.415 (6.22), 3.429 (6.72), 3.443 (4.02), 3.458 (5.30), 3.463 (5.85), 3.467 (5.76), 3.475 (6.95), 3.482 (15.91), 3.516 (15.68), 3.518 (16.00), 3.543 (1.46), 3.551 (2.06), 3.559 (3.75), 3.568 (4.89), 3.576 (4.02), 3.583 (3.43), 3.590 (3.20), 3.602 (3.89), 3.610 (3.15), 3.626 (0.64), 3.634 (1.01), 3.641 (1.01), 3.666 (0.55), 3.683 (0.46), 3.882 (0.55), 3.903 (0.69), 3.921 (0.55), 3.931 (0.50), 3.963 (0.41), 3.977 (0.41), 3.983 (0.55), 3.994 (0.78), 4.004 (0.55), 4.012 (0.46), 4.025 (0.64), 4.040 (0.46), 4.166 (0.32), 4.180 (0.37), 4.186 (0.55), 4.201 (0.55), 4.207 (0.41), 4.221 (0.37), 4.240 (0.50), 4.252 (0.55), 4.279 (0.37), 4.291 (0.37), 4.330 (0.87), 4.348 (0.59), 4.356 (0.55), 4.582 (0.50), 4.595 (0.82), 4.609 (0.41), 4.774 (0.27), 4.791 (0.64), 4.801 (0.27), 4.810 (0.64), 4.828 (0.32), 4.833 (0.73), 4.840 (1.28), 4.846 (0.73), 4.876 (0.69), 4.908 (0.55), 4.916 (0.87), 4.956 (0.50), 4.972 (1.01), 4.991 (1.05), 5.006 (0.50), 5.147 (0.78), 5.188 (0.64), 5.602 (1.65), 6.058 (0.87), 6.073 (1.65), 6.086 (0.82), 6.736 (1.10), 6.756 (1.19), 6.918 (1.60), 6.931 (1.05), 6.947 (2.01), 6.968 (2.51), 6.989 (0.59), 6.998 (0.46), 7.021 (0.27), 7.052 (0.50), 7.058 (0.55), 7.066 (0.55), 7.071 (0.59), 7.081 (1.97), 7.100 (2.74), 7.120 (1.23), 7.179 (2.01), 7.186 (3.20), 7.200 (4.43), 7.209 (6.54), 7.214 (3.79), 7.222 (3.25), 7.230 (6.58), 7.253 (2.65), 7.273 (2.79), 7.293 (3.43), 7.313 (1.60), 7.327 (1.78), 7.346 (2.10), 7.363 (0.87), 7.415 (1.01), 7.430 (0.27), 7.496 (1.28), 7.545 (0.37), 7.548 (0.37), 7.573 (1.33), 7.586 (0.96), 7.611 (0.64), 7.619 (0.64), 7.627 (0.59), 7.636 (0.78), 7.652 (1.28), 7.661 (0.73), 7.673 (0.91), 7.689 (0.32), 7.696 (0.37), 7.725 (1.05), 7.767 (0.32), 7.780 (0.69), 7.793 (1.10), 7.807 (0.64), 7.822 (0.32), 7.826 (0.27), 7.839 (0.32), 7.842 (0.32), 7.857 (0.50), 7.863 (0.55), 7.872 (0.55), 7.881 (0.59), 7.884 (0.59), 7.895 (0.87), 7.909 (1.19), 7.925 (1.42), 7.946 (1.01), 8.164 (0.78), 8.184 (0.78), 8.329 (3.02), 8.639 (1.05), 10.007 (0.27), 10.029 (0.50), 10.035 (0.50), 10.039 (0.50), 10.042 (0.50), 10.064 (0.32), 10.075 (0.27), 12.033 (1.65).

### Example 19: Preparation of sodium N²-(14-{4-[({(1R)-2-carboxylato-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)-L-asparaginyl-L-prolyl-N-[(2S)-4-[{(1R)-1-[1-benzyl-4-(2,5-difluorophenyl)-1H-pyrrol-2-yl]-2,2-dimethylpropyl}(hydroxyacetyl)amino]-1-(methylamino)-1-oxobutan-2-yl]-L-valinamide (Compound 19)

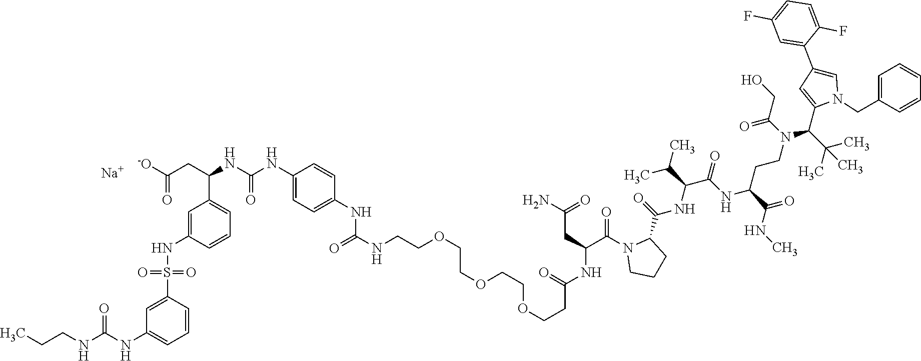

Step 1: To a solution of N²-(3-{2-[2-(2-aminoethoxy)ethoxy]ethoxy}propanoyl)-L-asparaginyl-L-prolyl-N-[(2S)-4-[{(1R)-1-[1-benzyl-4-(2,5-difluorophenyl)-1H-pyrrol-2-yl]-2,2-dimethylpropyl}(hydroxyacetyl)amino]-1-(methylamino)-1-oxobutan-2-yl]-L-valinamide (20.0 mg, 96% purity, 18.4 μmol) (Intermediate 47) in DMF (1.5 ml), were added (3R)-3-{[(4-{[(4-nitrophenoxy)carbonyl]amino)phenyl)carbamoyl]amino}-3-[3-((3-[(propylcarbamoyl)amino]benzene-1-sulfonyl}amino)phenyl]propanoic acid (14.5 mg, 20.2 μmol) (Intermediate 2) and DIEA (13 μl, 73 μmol). The mixture was stirred at rt for 30 min and then concentrated under reduced pressure. The residue was purified over preparative HPLC and freeze dried to afford (N²-(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)-L-asparaginyl-L-prolyl-N-[(2S)-4-[{(1R)-1-[1-benzyl-4-(2,5-difluorophenyl)-1H-pyrrol-2-yl]-2,2-dimethylpropyl}(hydroxyacetyl)amino]-1-(methylamino)-1-oxobutan-2-yl]-L-valinamide (23 mg, 93% purity, 72% yield) as an amorphous residue. LC-MS (Method 3): Rₜ=4.62 min; MS (ESIpos): m/z=1620 [M+H]⁺.

Step 2: To a solution of N²-(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)-L-asparaginyl-L-prolyl-N-[(2S)-4-[{(1R)-1-[1-benzyl-4-(2,5-difluorophenyl)-1H-pyrrol-2-yl]-2,2-dimethylpropyl}(hydroxyacetyl)amino]-1-(methylamino)-1-oxobutan-2-yl]-L-valinamide (23.0 mg, 93% purity, 13.3 μmol) in dioxane/water (1:1, 6 mL) was added a sodium hydroxide solution (13 μl, 1 M, 13 μmol). The solution was freeze-dried to give Compound 19 (22 mg, 94% purity, 95% yield) as a colorless foam. LC-MS (Method 3): Rₜ=4.64 min; MS (ESIpos): m/z=1621 [M+H]⁺. ¹H-NMR (400 MHz, DMSO-d6) delta [ppm]: 0.793 (3.00), 0.814 (16.00), 0.826 (8.00), 0.844 (3.17), 1.353 (1.67), 1.371 (1.50), 1.854 (1.17), 2.253 (2.83), 2.265 (2.83), 2.331 (1.67), 2.931 (1.33), 3.213 (1.83), 3.311 (2.33), 3.432 (2.67), 3.446 (1.67), 3.472 (2.67), 3.484 (2.33), 3.522 (10.50), 3.568 (6.50), 5.601 (1.67), 6.896 (1.50), 6.933 (1.00), 7.163 (2.00), 7.178 (3.00), 7.200 (3.33), 7.255 (3.17), 7.278 (2.17), 7.318 (1.83), 7.337 (2.17), 7.491 (1.17), 7.873 (1.00), 8.312 (1.50).

### Example 20: Preparation of sodium (4S)-4,11-diethyl-3,14-dioxo-3,4,12,14-tetrahydro-1H-pyrano[3′,4′:6,7]indolizino[1,2-b]quinolin-4-yl 1-[(2S)-15-{[N2,N6-bis(17-{[4-({2-[(2S)-2-cyano-4,4-difluoropyrrolidin-1-yl]-2-oxoethyl}carbamoyl)quinolin-8-yl]amino}-14,17-dioxo-4,7,10-trioxa-13-azaheptadecanan-1-oyl}-L-lysyl]amino)-2-(carboxylatomethyl)-4-oxo-7,10,13-trioxa-3-azapentadecanan-1-oyl]-L-prolyl-L-valinate (Compound 20)

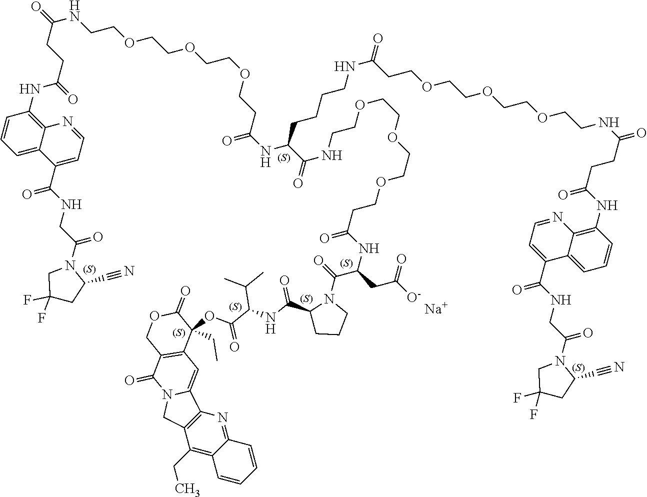

Step 1: To a solution of 4-{[4-({2-[(2S)-2-cyano-4,4-difluoropyrrolidin-1-yl]-2-oxoethyl}carbamoyl)quinolin-8-yl]amino}-4-oxobutanoic acid (15.9 mg, 34.5 μmol) in DMF (1.5 ml) and DCM (1.5 ml), were added HATU (13.1 mg, 34.5 μmol) and DIEA (24 μl, 140 μmol). After stirring for 15 min, (4S)-4,11-diethyl-3,14-dioxo-3,4,12,14-tetrahydro-1H-pyrano[3′,4′:6,7]indolizino[1,2-b]quinolin-4-yl N-(3-{2-[2-(2-{[N²,N⁶-bis(3-{2-[2-(2-aminoethoxy)ethoxy]ethoxy}propanoyl)-L-lysyl]amino}ethoxy)ethoxy]ethoxy}propanoyl)-L-alpha-aspartyl-L-prolyl-L-valinate.trifluoroacetic acid (1/2) (30.0 mg, 95% purity, 17.3 μmol)) (Intermediate 9) was added. The mixture was stirred at rt for 45 min and then concentrated under reduced pressure. The residue was purified over preparative HPLC and freeze dried to afford (4S)-4,11-diethyl-3,14-dioxo-3,4,12,14-tetrahydro-1H-pyrano[3′,4′:6,7]indolizino[1,2-b]quinolin-4-yl N-(3-{2-[2-(2-{[N²,N⁶-bis(17-{[4-({2-[(2S)-2-cyano-4,4-difluoropyrrolidin-1-yl]-2-oxoethyl}carbamoyl)quinolin-8-yl]amino}-14,17-dioxo-4,7,10-trioxa-13-azaheptadecanan-1-oyl)-L-lysyl]amino}ethoxy)ethoxy]ethoxy}propanoyl)-L-alpha-aspartyl-L-prolyl-L-valinate (20.9 mg, 98% purity, 51% yield). LC-MS (Method 3): Rₜ=4.06 min; MS (ESIpos): m/z=1154 [M+2H]²⁺.

Step 2: To a solution of (4S)-4,11-diethyl-3,14-dioxo-3,4,12,14-tetrahydro-1H-pyrano[3′,4′:6,7]indolizino[1,2-b]quinolin-4-yl N-(3-{2-[2-(2-{[N²,N⁶-bis(17-{[4-({2-[(2S)-2-cyano-4,4-difluoropyrrolidin-1-yl]-2-oxoethyl}carbamoyl)quinolin-8-yl]amino}-14,17-dioxo-4,7,10-trioxa-13-azaheptadecanan-1-oyl)-L-lysyl]amino}ethoxy)ethoxy]ethoxy}propanoyl)-L-alpha-aspartyl-L-prolyl-L-valinate (20.6 mg, 98% purity, 8.71 μmol) in dioxane/water (1:1, 4 mL) was added a sodium hydroxide solution (8.7 μl, 1.0 M, 8.7 μmol). The solution was freeze-dried to give Compound 20 (20.1 mg, 100% purity, 99% yield) as a colorless foam. LC-MS (Method 3): Rₜ=4.06 min; MS (ESIpos): m/z=2307 [M+H]⁺. ¹H-NMR (500 MHz, DMSO-d6) δ[ppm]: 0.91 (t, J=7.48 Hz, 4H) 0.95 (dd, J=6.64, 3.89 Hz, 5H) 1.01 (m, 1H) 1.22 (m, 3H) 1.30 (br t, J=7.63 Hz, 4H) 1.34 (m, 2H) 1.45 (m, 1H) 1.57 (m, 1H) 1.93 (m, 2H) 2.02 (m, 1H) 2.19 (br d, J=7.02 Hz, 4H) 2.28 (t, J=6.56 Hz, 3H) 2.36 (m, 5H) 2.48 (m, 4H) 2.52 (m, 2H) 2.54 (s, 7H) 2.64 (s, 1H) 2.80 (br t, J=7.02 Hz, 8H) 2.98 (br s, 4H) 3.20 (m, 12H) 3.38 (m, 11H) 3.47 (m, 25H) 3.57 (m, 14H) 3.72 (m, 1H) 3.75 (m, 1H) 4.09 (m, 1H) 4.14 (br d, J=11.29 Hz, 1H) 4.17 (br s, 1H) 4.19 (br s, 2H) 4.23 (s, 1H) 4.25 (d, J=6.26 Hz, 2H) 4.29 (m, 1H) 4.34 (m, 1H) 4.84 (m, 1H) 4.92 (q, J=7.07 Hz, 1H) 5.18 (dd, J=9.23, 2.36 Hz, 2H) 5.33 (br d, J=6.26 Hz, 2H) 5.49 (s, 2H) 7.25 (br s, 1H) 7.62 (m, 1H) 7.65 (d, J=4.43 Hz, 2H) 7.73 (t, J=7.71 Hz, 1H) 7.78 (br t, J=5.57 Hz, 1H) 7.86 (s, 1H) 7.90 (br s, 1H) 7.93 (br d, J=8.24 Hz, 1H) 7.98 (br d, J=7.32 Hz, 5H) 8.05 (d, J=8.09 Hz, 1H) 8.15 (br d, J=7.17 Hz, 1H) 8.29 (d, J=8.24 Hz, 1H) 8.37 (br d, J=8.24 Hz, 1H) 8.64 (d, J=7.63 Hz, 2H) 9.00 (d, J=4.42 Hz, 2H) 9.14 (br t, J=5.87 Hz, 2H) 10.14 (s, 2H) 12.40 (m, 1H).

### Example 21: Preparation of disodium (4S)-4,11-diethyl-3,14-dioxo-3,4,12,14-tetrahydro-1H-pyrano[3′,4′:6,7]indolizino[1,2-b]quinolin-4-yl 1-[(2S,27S)-27-[(7-carboxylatoheptyl)carbamoyl]-2-(carboxylatomethyl)-45-{[4-({2-[(2S)-2-cyano-4,4-difluoropyrrolidin-1-yl]-2-oxoethyl}carbamoyl)quinolin-8-yl]amino}-4,17,21,29,42,45-hexaoxo-7,10,13,32,35,38-hexaoxa-3,16,22,28,41-pentaazapentatetracontanan-1-oyl]-L-prolyl-L-valinate (Compound 21)

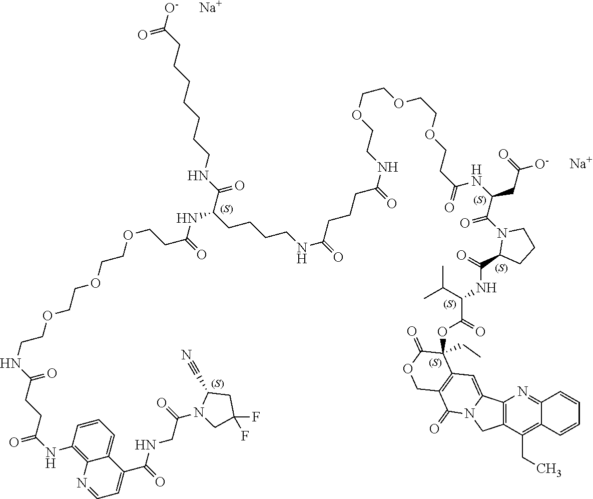

Step 1: To a solution of 4-{[4-({2-[(2S)-2-cyano-4,4-difluoropyrrolidin-1-yl]-2-oxoethyl}carbamoyl)quinolin-8-yl]amino}-4-oxobutanoic acid (7.69 mg, 100% purity, 16.7 μmol) in DMF (1.5 ml) and DCM (1.5 ml), were added HATU (6.37 mg, 16.7 μmol) and DIEA (12 μl, 67 μmol). After stirring for 15 min, (4S)-4,11-diethyl-3,14-dioxo-3,4,12,14-tetrahydro-1H-pyrano[3′,4′:6,7]indolizino[1,2-b]quinolin-4-yl N-{(24S)-37-amino-24-[(7-carboxyheptyl)carbamoyl]-14,18,26-trioxo-4,7,10,29,32,35-hexaoxa-13,19,25-triazaheptatriacontanan-1-oyl}-L-alpha-aspartyl-L-prolyl-L-valinate.trifluoroacetic acid (1/2) (30.0 mg, 95% purity, 16.7 μmol) (Intermediate 53) was added. The mixture was stirred overnight at rt. A solution of 4-{[4-({2-[(2S)-2-cyano-4,4-difluoropyrrolidin-1-yl]-2-oxoethyl}carbamoyl)quinolin-8-yl]amino}-4-oxobutanoic acid (7.69 mg, 100% purity, 16.7 μmol), HATU (6.37 mg, 16.7 μmol) and DIEA (12 μl, 67 μmol) in DMF (1.5 ml) and DCM (1.5 ml) was stirred for 15 min and then added to the reaction mixture. After 30 min, the mixture was concentrated under reduced pressure. The residue was purified over preparative HPLC and freeze dried to afford of (4S)-4,11-diethyl-3,14-dioxo-3,4,12,14-tetrahydro-1H-pyrano[3′,4′:6,7]indolizino[1,2-b]quinolin-4-yl N-[(24S)-24-[(7-carboxyheptyl)carbamoyl]-42-{[4-({2-[(2S)-2-cyano-4,4-difluoropyrrolidin-1-yl]-2-oxoethyl}carbamoyl)quinolin-8-yl]amino}-14,18,26,39,42-pentaoxo-4,7,10,29,32,35-hexaoxa-13,19,25,38-tetraazadotetracontanan-1-oyl]-L-alpha-aspartyl-L-prolyl-L-valinate (17.1 mg, 100% purity, 53% yield). LC-MS (Method 3): Rₜ=3.94 min; MS (ESIpos): m/z=1919 [M+H]⁺.

Step 2: To a solution of (4S)-4,11-diethyl-3,14-dioxo-3,4,12,14-tetrahydro-1H-pyrano[3′,4′:6,7]indolizino[1,2-b]quinolin-4-yl N-[(24S)-24-[(7-carboxyheptyl)carbamoyl]-42-{[4-({2-[(2S)-2-cyano-4,4-difluoropyrrolidin-1-yl]-2-oxoethyl}carbamoyl)quinolin-8-yl]amino}-14,18,26,39,42-pentaoxo-4,7,10,29,32,35-hexaoxa-13,19,25,38-tetraazadotetracontanan-1-oyl]-L-alpha-aspartyl-L-prolyl-L-valinate (16.8 mg, 100% purity, 8.75 μmol) in dioxane/water (1:1, 4 mL) was added a sodium hydroxide solution (18 μl, 1 M, 18 μmol). The solution was freeze-dried to give Compound 21 (16.8 mg, 96% purity, 94% yield) as a colorless foam. LC-MS (Method 3): Rₜ=3.94 min; MS (ESIpos): m/z=1917 [M+H]⁺. ¹H-NMR (500 MHz, DMSO-d6) δ[ppm]: 0.87 (m, 6H) 0.92 (d, J=6.56 Hz, 3H) 1.21 (br s, 9H) 1.31 (m, 5H) 1.35 (br s, 4H) 1.44 (br d, J=6.87 Hz, 5H) 1.57 (m, 1H) 1.67 (br t, J=7.32 Hz, 2H) 1.80 (m, 1H) 1.96 (m, 2H) 2.03 (m, 7H) 2.14 (br dd, J=14.72, 4.20 Hz, 1H) 2.25 (m, 4H) 2.37 (m, 4H) 2.48 (br s, 1H) 2.54 (m, 5H) 2.64 (br d, J=1.83 Hz, 1H) 2.69 (br dd, J=15.18, 10.30 Hz, 1H) 2.80 (br t, J=7.02 Hz, 4H) 2.97 (m, 5H) 3.05 (m, 2H) 3.15 (m, 4H) 3.21 (m, 8H) 3.43 (br d, J=3.81 Hz, 13H) 3.47 (br d, J=2.29 Hz, 12H) 3.57 (m, 11H) 3.57 (m, 1H) 3.71 (m, 3H) 3.86 (br d, J=7.78 Hz, 2H) 4.01 (br s, 2H) 4.16 (br d, J=5.80 Hz, 3H) 4.24 (br t, J=6.18 Hz, 2H) 4.31 (m, 2H) 4.78 (m, 1H) 5.18 (br d, J=7.02 Hz, 1H) 5.30 (br d, J=9.92 Hz, 1H) 5.33 (br d, J=3.81 Hz, 2H) 5.45 (s, 2H) 7.64 (m, 2H) 7.71 (m, 2H) 7.86 (br d, J=8.09 Hz, 1H) 7.91 (br d, J=5.95 Hz, 2H) 7.99 (dd, J=8.16, 4.35 Hz, 3H) 8.05 (br s, 1H) 8.13 (br d, J=7.78 Hz, 2H) 8.28 (d, J=8.09 Hz, 1H) 8.65 (d, J=7.02 Hz, 1H) 9.00 (d, J=4.27 Hz, 2H) 9.22 (s, 1H) 10.15 (s, 1H).

### Example 22: Preparation of disodium 1-{(2S)-2-(carboxylatomethyl)-17-[4-(([(1R)-2-carboxylato-1-{3-[({3-[(propylcarbamoyl)amino]phenyl}sulfonyl)amino]phenyl)ethyl]carbamoyl}amino)anilino]-4,17-dioxo-7,10,13-trioxa-3,16-diazaheptadecan-1-oyl}-L-prolyl-N-[2-(ethoxymethyl)-1-(2-hydroxy-2-methylpropyl)-1H-imidazo[4,5-c]quinolin-4-yl]-L-valinamide (Compound 22)

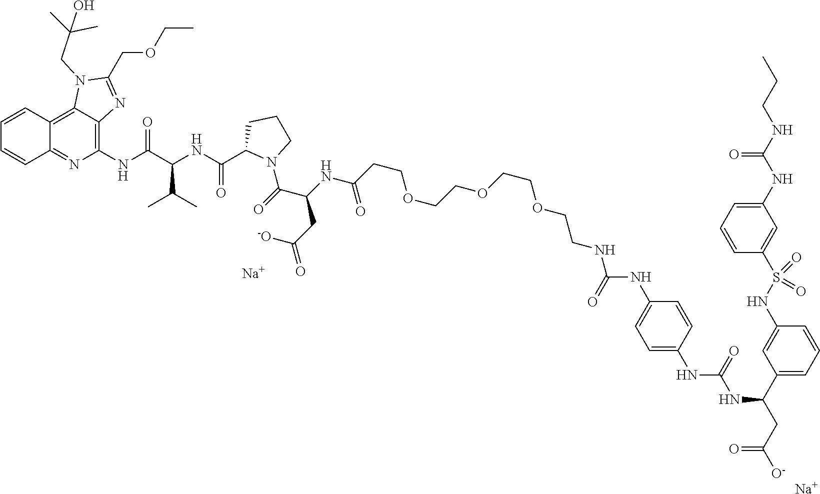

Intermediate 64 (58.0 mg, 41.1 umol) was dissolved in dioxane/water (1:1, 10.0 ml) and a 1M sodium hydroxide solution (82.3 ul) was added. The solution was freeze-dried to give Compound 22 (58.0 mg, 100% purity, 97% yield) as a colorless foam. LC-MS (Method 6): Rₜ=2.46 min; MS (ESIpos): m/z=705 [M+2H]².

### Example 23: Preparation of N-{[(2-[{2-[{[4-({[(1R)-2-carboxy-1-{3-[({3-[(propylcarbamoyl)amino]phenyl}sulfonyl)amino]phenyl}ethyl]carbamoyl}amino)phenyl]carbamoyl}(methyl)amino]ethyl)(methyl)amino]ethyl}(methyl)amino]acetyl}-L-asparaginyl-L-prolyl-N-[2-(ethoxymethyl)-1-(2-hydroxy-2-methylpropyl)-1H-imidazo[4,5-c]quinolin-4-yl]-L-valinamide (Compound 23)

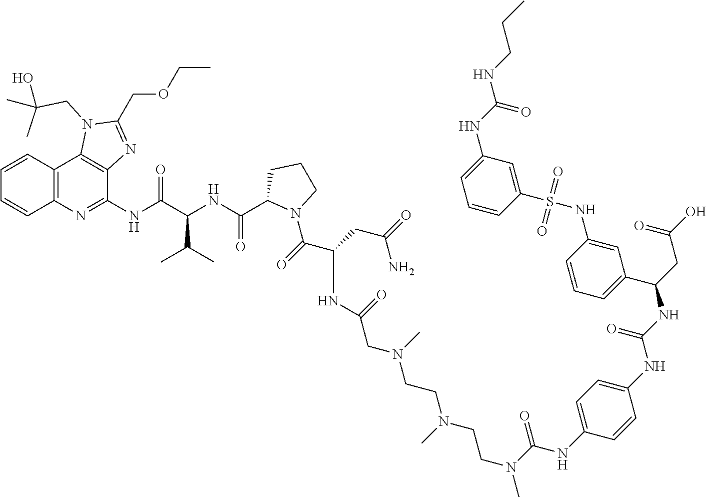 [SMILES: CCCNC(=O)Nc1cccc(S(=O)(=O)Nc2cccc([C@@H](CC(=O)O)NC(=O)Nc3ccc(NC(=O)N(C)CCN(C)CCN(C)CC(=O)N[C@@H](CC(N)=O)C(=O)N4CCC[C@H]4C(=O)N[C@H](C(=O)Nc4nc5ccccc5c5c4nc(COCC)n5CC(C)(C)O)C(C)C)cc3)c2)c1]

To a solution of Intermediate 55 (10.0 mg, 10.8 umol) in DMF (6.0 ml) were added Example S2 (14.0 mg, 19.5 umol) and DIEA (9.43 ul, 54.1 umol). The reaction was stirred overnight at RT, concentrated in vacuo. The residue was purified by prep. HPLC, concentrated and lyophilized to give Compound 23 (16.0 mg, 93% purity, 99% yield) as a white foam. LC-MS (Method 6): Rt=2.15 min; MS (ESIpos): m/z=1390 [M+H]+.

### Example 24: Preparation of N-{[{2-[{2-[{[4-({[(1S)-2-carboxy-1-{3-[({3-[(propylcarbamoyl)amino]phenyl}sulfonyl)amino]phenyl}ethyl]carbamoyl}amino)phenyl]carbamoyl}(methyl)amino]ethyl}(methyl)amino]ethyl}(methyl)amino]acetyl}-L-asparaginyl-L-prolyl-N-[2-(ethoxymethyl)-1-(2-hydroxy-2-methylpropyl)-1H-imidazo[4,5-c]quinolin-4-yl]-L-valinamide (Compound 24)

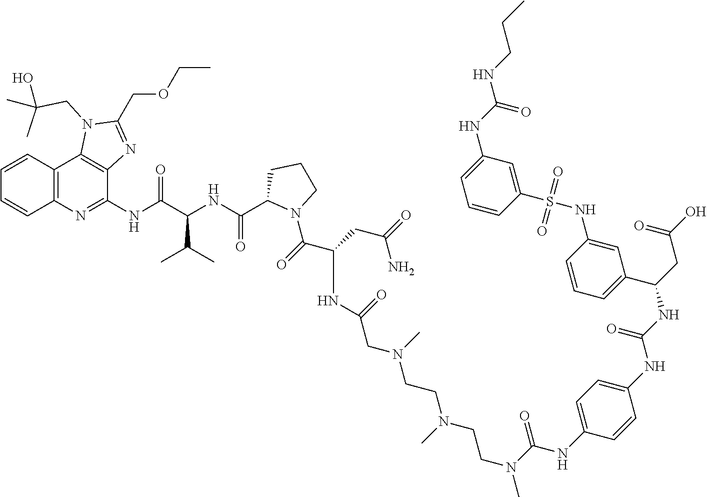 [SMILES: CCCNC(=O)Nc1cccc(S(=O)(=O)Nc2cccc([C@H](CC(=O)O)NC(=O)Nc3ccc(NC(=O)N(C)CCN(C)CCN(C)CC(=O)N[C@@H](CC(N)=O)C(=O)N4CCC[C@H]4C(=O)N[C@H](C(=O)Nc4nc5ccccc5c5c4nc(COCC)n5CC(C)(C)O)C(C)C)cc3)c2)c1]

To a solution of Intermediate 55 (10.0 mg, 10.8 umol) in DMF (6.0 ml) were added Intermediate 54 (14.0 mg, 19.5 umol) and DIEA (9.43 ul, 54.1 umol). The reaction was then stirred overnight at RT and concentrated in vacuo. The residue was purified by prep. HPLC, then concentrated and lyophilized to give Compound 24 (14.0 mg, 87% purity, 81% yield) as a yellow foam. LC-MS (Method 3): Rₜ=3.01 min; MS (ESIpos): m/z=1390 [M+H]⁺.

### Example 25: Preparation of disodium 1-[(2S)-2-(carboxylatomethyl)-17-{4-[({(1R)-2-carboxylato-1-[3-({3-[(propylcarbamoyl)amino]benzene-1-sulfonyl}amino)phenyl]ethyl}carbamoyl)amino]anilino}-4,17-dioxo-7,10,13-trioxa-3,16-diazaheptadecanan-1-oyl]-L-prolyl-N-{3-[({1R)-1-[1-benzyl-4-(2,5-difluorophenyl)-1H-pyrrol-2-yl]-2,2-dimethylpropyl}(hydroxyacetyl)amino]propyl}-L-valinamide (Compound 25)

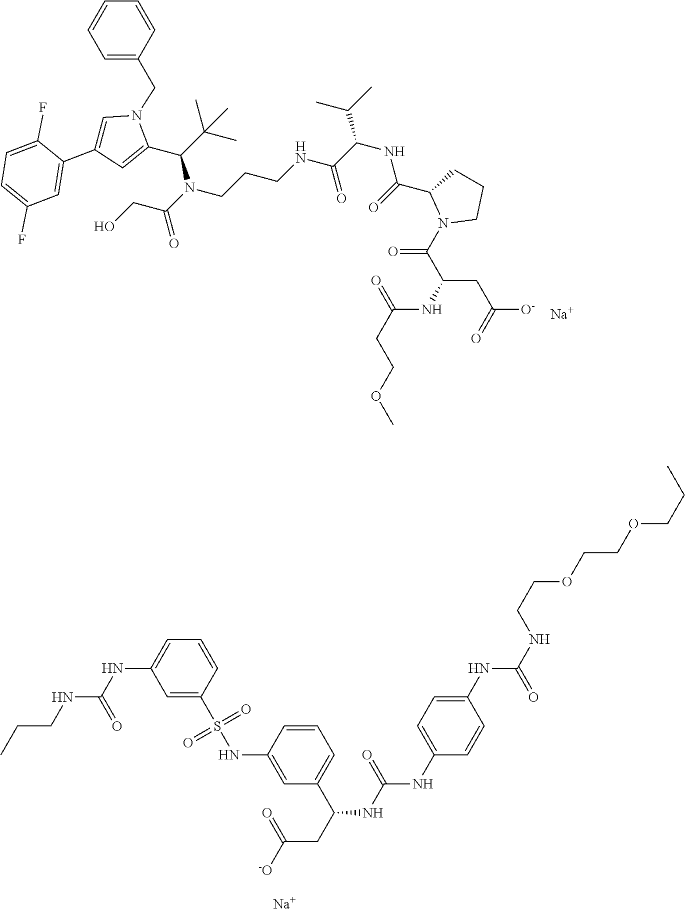

Step I: To a solution of N-(3-{2-[2-(2-aminoethoxy)ethoxy]ethoxy}propanoyl)-L-alpha-aspartyl-L-prolyl-N-{3-[{(1R)-1-[1-benzyl-4-(2,5-difluorophenyl)-1H-pyrrol-2-yl]-2,2-dimethylpropyl}(hydroxyacetyl)amino]propyl}-L-valinamide.trifiuoroacetic acid (1/1)(20.0 mg, 98% purity, 17.9 μmol) (Intermediate 73) in DMF (2.0 ml), were added (3R)-3-{[(4-{[(4-nitrophenoxy)carbonyl]amino}phenyl)carbamoyl]amino}-3-[3-({3-[(propylcarbamoyl)amino]benzene-1-sulfonyl}amino)phenyl]propanoic acid (12.9 mg, 17.9 μmol) (Intermediate 2) and DIEA (31 μl, 180 μmol). The mixture was stirred at rt for 1 h and then concentrated under reduced pressure. The residue was purified over preparative HPLC and freeze dried to afford N-(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)-L-alpha-aspartyl-L-prolyl-N-{3-[{(1R)-1-[1-benzyl-4-(2,5-difluorophenyl)-1H-pyrrol-2-yl]-2,2-dimethylpropyl}(hydroxyacetyl)amino]propyl}-L-valinamide (20 mg, 100% purity, 72% yield) as a colorless foam. LC-MS (Method 3): Rₜ=4.86 min; MS (ESIpos): m/z=1565 [M+H]⁺.

Step 2: To a solution of N-(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)-L-alpha-aspartyl-L-prolyl-N-{3-[{(1R)-1-[1-benzyl-4-(2,5-difluorophenyl)-1H-pyrrol-2-yl]-2,2-dimethylpropyl}(hydroxyacetyl)amino]propyl}-L-valinamide (20.0 mg, 26 μmol) in dioxane/water (1:1, 4 mL) was added a sodium hydroxide solution (26 μl, 1 M, 13 μmol). The solution was freeze-dried to give Compound 25 (23 mg, 94% purity, quant.) as a colorless foam. MS (Method 3): Rₜ=4.86 min; MS (ESIpos): m/z=1563 [M+H]⁺.

### Example 26: Preparation of disodium 1-[(2S)-2-(carboxylatomethyl)-17-{4-[({(1R)-2-carboxylato-1-[3-({3-[(propylcarbamoyl)amino]benzene-1-sulfonyl}amino)phenyl]ethyl}carbamoyl)amino]anilino}-4,17-dioxo-7,10,13-trioxa-3,16-diazaheptadecanan-1-oyl]-L-prolyl-N-[(2S)-4-[{(1R)-1-[1-benzyl-4-(2,5-difluorophenyl)-1H-pyrrol-2-yl]-2,2-dimethylpropyl}(hydroxyacetyl)amino]-1-(methylamino)-1-oxobutan-2-yl]-L-valinamide (Compound 26)

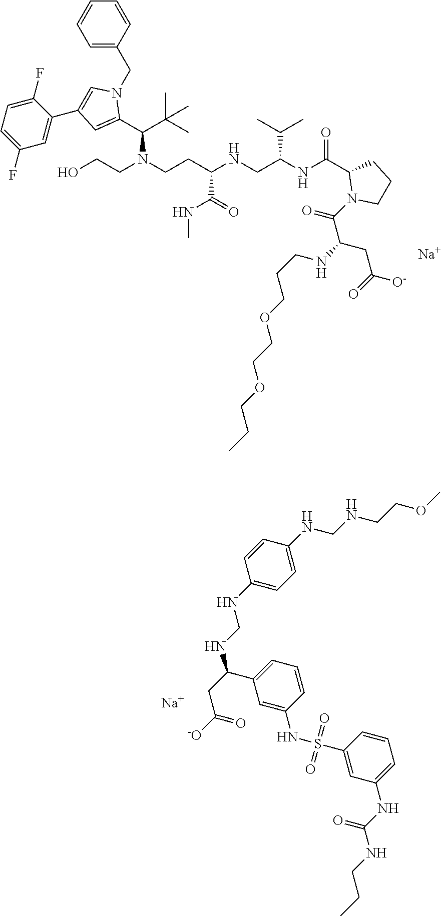

Step 1: To a solution of N-(3-{2-[2-(2-aminoethoxy)ethoxy]ethoxy}propanoyl)-L-alpha-aspartyl-L-prolyl-N-[(2S)-4-[{(1R)-1-[1-benzyl-4-(2,5-difluorophenyl)-1H-pyrrol-2-yl]-2,2-dimethylpropyl}(hydroxyacetyl)amino]-1-(methylamino)-1-oxobutan-2-yl]-L-valinamide.trifluoroacetic acid (1/1) (20.0 mg, 100% purity, 17.3 μmol) (Intermediate 72) in DMF (2.1 ml), were added (3R)-3-{[(4-{[(4-nitrophenoxy) carbonyl]amino}phenyl) carbamoyl]amino}-3-[3-({3-[(propylcarbamoyl)amino]benzene-1-sulfonyl}amino)phenyl]propanoic acid (14.4 mg, 95% purity, 19.0 μmol) (Intermediate 2) and DIEA (30 μl, 170 μmol). The mixture was stirred at rt for 1 h and then concentrated under reduced pressure. The residue was purified over preparative HPLC and freeze dried to afford N-(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)-L-alpha-aspartyl-L-prolyl-N-[(2S)-4-[{(1R)-1-[1-benzyl-4-(2,5-difluorophenyl)-1H-pyrrol-2-yl]-2,2-dimethylpropyl}(hydroxyacetyl)amino]-1-(methylamino)-1-oxobutan-2-yl]-L-valinamide (22.3 mg, 99% purity, 78% yield). LC-MS (Method 3): Rₜ=4.72 min; MS (ESIpos): m/z=1622 [M+H]⁺.

Step 2: To a solution of N-(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)-L-alpha-aspartyl-L-prolyl-N-[(2S)-4-[{(1R)-1-[1-benzyl-4-(2,5-difluorophenyl)-1H-pyrrol-2-yl]-2,2-dimethylpropyl}(hydroxyacetyl)amino]-1-(methylamino)-1-oxobutan-2-yl]-L-valinamide (21.8 mg, 99% purity, 13.2 μmol) in dioxane/water (1:1, 6 mL) was added a sodium hydroxide solution (26 μl, 1 M, 26 μmol). The solution was freeze-dried to give Compound 26 (21.8 mg, 100% purity, 99% yield) as a colorless foam. LC-MS (Method 3): Rₜ=4.72 min; MS (ESIpos): m/z=1620 [M+H]⁺. ¹H-NMR (400 MHz, DMSO-d6) delta [ppm]: 0.74-0.86 (m, 2H), 1.39 (q, 1H), 1.86-1.92 (m, 1H), 1.98-2.03 (m, 1H), 2.26-2.38 (m, 1H), 2.45 (br d, 1H), 2.54 (s, 5H), 2.58-2.61 (m, 1H), 2.67 (t, 1H), 2.95-3.00 (m, 1H), 3.08-3.14 (m, 1H), 3.16-3.32 (m, 1H), 3.40-3.52 (m, 2H), 3.56-3.60 (m, 1H), 3.73-3.78 (m, 1H), 4.29-4.32 (m, 1H), 5.61 (s, 1H), 6.93-6.98 (m, 1H), 7.12-7.34 (m, 1H), 7.46 (br s, 1H), 7.77-7.81 (m, 1H), 8.38 (s, 1H).

### Example 27: Preparation of trisodium 1-[(2S)-15-{[N²,N⁶-bis(14-{4-[({(1R)-2-carboxylato-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl}-L-lysyl]amino)-2-(carboxylatomethyl)-4-oxo-7,10,13-trioxa-3-azapentadecanan-1-oyl]-L-prolyl-N-[(2S)-4-[{(1R)-1-[1-benzyl-4-(2,5-difluorophenyl)-1H-pyrrol-2-yl]-2,2-dimethylpropyl}(hydroxyacetyl)amino]-1-(methylamino)-1-oxobutan-2-yl]-L-valinamide (Compound 27)

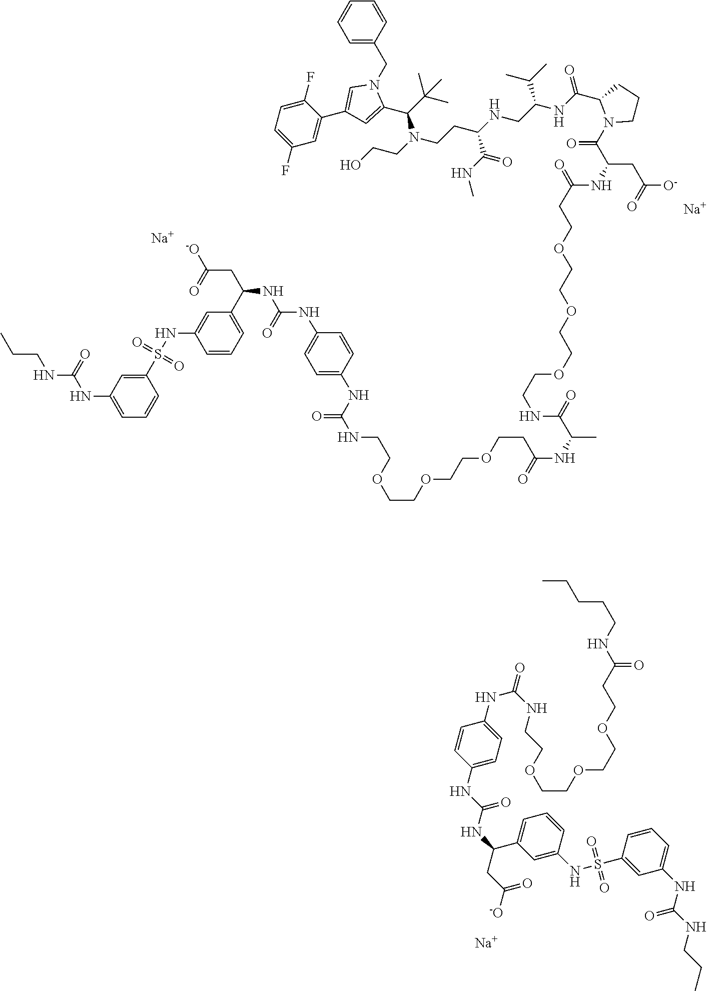

Step 1: To a solution of N-(3-{2-[2-(2-{[N2,N6-bis(3-{2-[2-(2-aminoethoxy)ethoxy]ethoxy}propanoyl)-L-lysyl]amino}ethoxy)ethoxy]ethoxy}propanoyl)-L-alpha-aspartyl-L-prolyl-N-[(2S)-4-[{((1R)-1-[1-benzyl-4-(2,5-difluorophenyl)-1H-pyrrol-2-yl]-2,2-dimethylpropyl}(hydroxyacetyl)amino]-1-(methylamino)-1-oxobutan-2-yl]-L-valinamide.trifluoroacetic acid (1/2) (250 mg, 139 μmol) (Intermediate 75) in DMF (8.0 ml), were added (3R)-3-{[(4-{[(4-nitrophenoxy)carbonyl]amino}phenyl)carbamoyl]amino}-3-[3-({3-[(propylcarbamoyl)amino]benzene-1-sulfonyl}amino)phenyl]propanoic acid (231 mg, 95% purity, 305 μmol) (Intermediate 2) and DIEA (240 μl, 1.4 mmol). The mixture was stirred at rt for 1 h and then concentrated under reduced pressure. The residue was purified over preparative HPLC and freeze dried to afford N-(3-{2-[2-(2-{[N2,N6-bis(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl}-L-lysyl]amino)ethoxy)ethoxy]ethoxy}propanoyl)-L-alpha-aspartyl-L-prolyl-N-[(2S)-4-[{(1R)-1-[1-benzyl-4-(2,5-difluorophenyl)-1H-pyrrol-2-yl]-2,2-dimethylpropyl}(hydroxyacetyl)amino]-1-(methylamino)-1-oxobutan-2-yl]-L-valinamide (319 mg, 97% purity, 81% yield) as a colorless foam. LC-MS (Method 3): Rₜ=4.74 min; MS: m/z=1369 [M−H]⁻.

Step 2: To a solution N-(3-{2-[2-(2-{[N²,N⁶-bis(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)-L-lysyl]amino}ethoxy)ethoxy]ethoxy}propanoyl)-L-alpha-aspartyl-L-prolyl-N-[(2S)-4-[{(1R)-1-[1-benzyl-4-(2,5-difluorophenyl)-1H-pyrrol-2-yl]-2,2-dimethylpropyl}(hydroxyacetyl)amino]-1-(methylamino)-1-oxobutan-2-yl]-L-valinamide (318 mg, 97% purity, 112 μmol) in dioxane/water (1:1, 50 mL) was added a sodium hydroxide solution (340 μl, 1 M, 340 μmol). The mixture was dissolved in an ultrasonic batch and then freeze-dried to give Compound 27 (328 mg, 96% purity, 100% yield) as a white foam. LC-MS (Method 3): Rₜ=4.72 min; MS (ESIpos): m/z=1368 [M+2H]²⁺. ¹H-NMR (400 MHz, DMSO-d6) delta [ppm]: 0.61-0.91 (m, 6H), 1.16-1.27 (m, 1H), 1.28-1.48 (m, 2H), 1.79-2.08 (m, 2H), 2.19-2.43 (m, 3H), 2.54-2.55 (m, 2H), 2.55 (br s, 1H), 2.56-2.78 (m, 2H), 2.90-3.16 (m, 3H), 3.19-3.25 (m, 2H), 3.41-3.54 (m, 9H), 3.57 (s, 2H), 3.58 (br s, 1H), 3.59-3.71 (m, 1H), 3.88-4.05 (m, 1H), 4.07-4.24 (m, 1H), 4.76-5.02 (m, 1H), 6.00-6.12 (m, 1H), 6.32-6.56 (m, 1H), 6.68-6.90 (m, 1H), 6.90-7.00 (m, 1H), 7.04-7.19 (m, 2H), 7.21 (s, 2H), 7.22-7.35 (m, 2H), 7.49 (br s, 1H), 7.65-7.89 (m, 1H), 7.89-7.97 (m, 1H), 7.99 (br s, 1H), 8.00-8.15 (m, 1H), 8.34 (s, 1H), 8.35-8.43 (m, 1H), 10.19-10.27 (m, 1H).

### Example 28: Preparation of (4S)-4,11-diethyl-3,14-dioxo-3,4,12,14-tetrahydro-1H-pyrano[3′,4′:6,7]indolizino[1,2-b]quinolin-4-yl N-(26-{[4-(4-{(1S)-2-carboxy-1-[(N-{5-[(4-methylpyridin-2-yl)amino]pentanoyl}glycyl)amino]ethyl}phenyl)naphthalen-1-yl]oxy}-14-oxo-4,7,10,18,21,24-hexaoxa-13,15-diazahexacosanan-1-oyl)-L-alpha-aspartyl-L-prolyl-L-valinate (Compound 28)

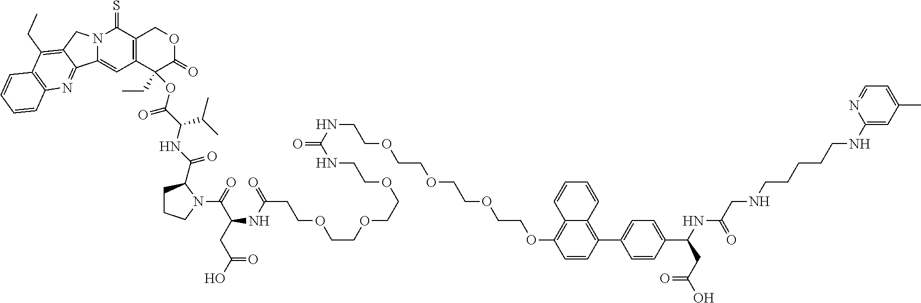 [SMILES: CCc1c2c(nc3ccccc13)-c1cc3c(c(=S)n1C2)COC(=O)[C@@]3(CC)OC(=O)[C@@H](NC(=O)[C@@H]1CCCN1C(=O)[C@H](CC(=O)O)NC(=O)CCOCCOCCOCCNC(=O)NCCOCCOCCOCCOc1ccc(-c2ccc([C@H](CC(=O)O)NC(=O)CNCCCCCNc3cc(C)ccn3)cc2)c2ccccc12)C(C)C]

Step 1: To a solution of (4S)-4,11-diethyl-3,14-dioxo-3,4,12,14-tetrahydro-1H-pyrano[3′,4′:6,7]indolizino[1,2-b]quinolin-4-yl N-[14-(4-nitrophenoxy)-14-oxo-4,7,10-trioxa-13-azatetradecanan-1-oyl]-L-alpha-aspartyl-L-prolyl-L-valinate (6.00 mg, 5.68 μmol) (Intermediate 76) in DMF (100 μl), were added (3S)-3-{4-[4-(2-{2-[2-(2-aminoethoxy)ethoxy]ethoxy}ethoxy)naphthalen-1-yl]phenyl}-3-[(N-{5-[(4-methylpyridin-2-yl)amino]pentanoyl}glycyl)amino]propanoic acid (4.15 mg, 5.68 μmol) (Intermediate 78) and DIEA (9.9 μl, 57 μmol). The mixture was stirred overnight at rt and then concentrated under reduced pressure. The residue was purified over preparative HPLC and freeze dried to afford Compound 28 (3.0 mg, 100% purity, 32% yield) as a white foam. LC-MS (Method 3): Rₜ=3.68 min; MS (ESIpos): m/z=1647 [M+H]⁺. ¹H-NMR (400 MHz, DMSO-d6) delta [ppm]: 0.88-1.03 (m, 5H), 1.30 (t, 2H), 1.46-1.62 (m, 2H), 1.85-2.03 (m, 2H), 2.08-2.28 (m, 5H), 2.31-2.48 (m, 3H), 2.52-2.56 (m, 9H), 2.67 (dt, 1H), 2.71-2.83 (m, 2H), 2.97-3.25 (m, 12H), 3.44-3.64 (m, 15H), 3.66-3.77 (m, 3H), 3.89-3.94 (m, 1H), 4.08 (t, 1H), 4.29-4.33 (m, 1H), 4.84 (dd, 1H), 4.89-4.95 (m, 1H), 5.25-5.34 (m, 1H), 5.49 (s, 1H), 5.97 (br t, 1H), 6.26-6.30 (m, 1H), 7.04 (d, 1H), 7.24 (s, 1H), 7.32 (d, 1H), 7.36-7.40 (m, 1H), 7.42-7.55 (m, 2H), 7.71-7.78 (m, 2H), 7.86 (t, 1H), 8.03-8.10 (m, 1H), 8.16 (br d, 1H), 8.23-8.30 (m, 1H), 8.52 (br d, 1H).

### Example 29: Preparation of disodium (4S)-4,11-diethyl-3,14-dioxo-3,4,12,14-tetrahydro-1H-pyrano[3′,4′:6,7]indolizino[1,2-b]quinolin-4-yl 1-{(2S,20S)-20-{[(1S)-2-carboxylato-1-(3,5-dichlorophenyl)ethyl]carbamoyl}-2-(carboxylatomethyl)-26-[(4-methylpyridin-2-yl)amino]-4,17,22-trioxo-7,10,13-trioxa-3,16,18,21-tetraazahexacosanan-1-oyl}-L-prolyl-L-valinate (Compound 29)

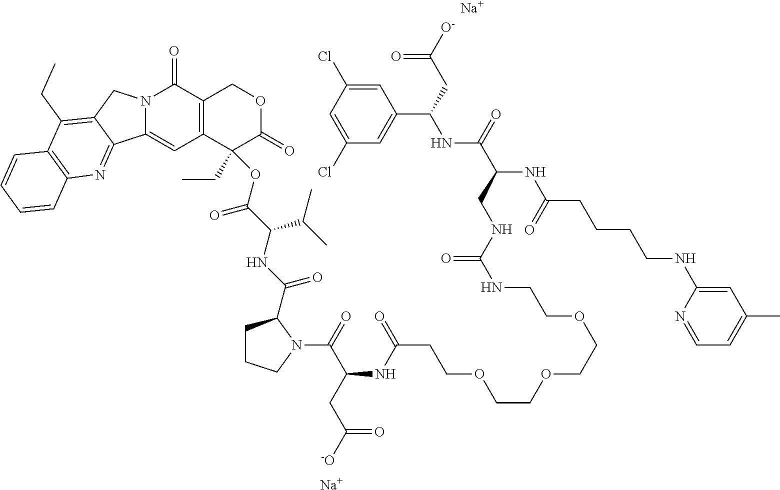

Step 1: To a solution of (3S)-3-[(N-{5-[(tert-butoxycarbonyl)(4-methylpyridin-2-yl)amino]pentanoyl}-3-{[(4-nitrophenoxy)carbonyl]amino}-L-alanyl)amino]-3-(3,5-dichlorophenyl)propanoic acid (28.0 mg, 95% purity, 34.1 μmol) (Intermediate 80) in DMF (0.3 ml), were added 4S)-4,11-diethyl-3,14-dioxo-3,4,12,14-tetrahydro-1H-pyrano[3′,4′:6,7]indolizino[1,2-b]quinolin-4-yl N-(3-{2-[2-(2-aminoethoxy)ethoxy]ethoxy}propanoyl)-L-alpha-aspartyl-L-prolyl-L-valinate.trifiuoroacetic acid (1/1) (34.3 mg, 34.1 μmol) (Intermediate 6) and DIEA (59 μl, 340 μmol). The mixture was stirred overnight at rt and then concentrated under reduced pressure. The residue was purified over preparative HPLC and freeze dried to afford (4S)-4,11-diethyl-3,14-dioxo-3,4,12,14-tetrahydro-1H-pyrano[3′,4′:6,7]indolizino[1,2-b]quinolin-4-yl N-[(12S)-12-{[(1S)-2-carboxy-1-(3,5-dichlorophenyl)ethyl]carbamoyl}-2,2-dimethyl-5-(4-methylpyridin-2-yl)-4,10,15,28-tetraoxo-3,19,22,25-tetraoxa-5,11,14,16-tetraazaoctacosan-28-yl]-L-alpha-aspartyl-L-prolyl-L-valinate (15 mg, 95% purity, 27% yield) as a colorless foam. LC-MS (Method 3): R: =4.86 min; MS (ESIpos): mi/z=1528 [M+H]⁺.

Step 2: To a solution of 4S)-4,11-diethyl-3,14-dioxo-3,4,12,14-tetrahydro-1H-pyrano[3′,4′:6,7]indolizino[1,2-b]quinolin-4-yl N-[(12S)-12-{[(1S)-2-carboxy-1-(3,5-dichlorophenyl)ethyl]carbamoyl}-2,2-dimethyl-5-(4-methylpyridin-2-yl)-4,10,15,28-tetraoxo-3,19,22,25-tetraoxa-5,11,14,16-tetraazaoctacosan-28-yl]-L-alpha-aspartyl-L-prolyl-L-valinate (14.0 mg, 95% purity, 8.72 μmol) in DCM (800 μl) was added TFA (140 μl). The mixture was stirred overnight at rt and then concentrated under reduced pressure. The residue was purified over preparative HPLC and freeze dried to afford (4S)-4,11-diethyl-3,14-dioxo-3,4,12,14-tetrahydro-1H-pyrano[3′,4′:6,7]indolizino[1,2-b]quinolin-4-yl N-{(17S)-17-{[(1S)-2-carboxy-1-(3,5-dichlorophenyl)ethyl]carbamoyl}-23-[(4-methylpyridin-2-yl)amino]-14,19-dioxo-4,7,10-trioxa-13,15,18-triazatricosanan-1-oyl}-L-alpha-aspartyl-L-prolyl-L-valinate (10 mg, 100% purity, 80% yield). LC-MS (Method 3): Rₜ=3.72 min; MS (ESIpos): m/z=1427 [M+H]⁺.

Step 3: To a solution of (4S)-4,11-diethyl-3,14-dioxo-3,4,12,14-tetrahydro-1H-pyrano[3′,4′:6,7]indolizino[1,2-b]quinolin-4-yl N-{(17S)-17-{[(1S)-2-carboxy-1-(3,5-dichlorophenyl)ethyl]carbamoyl}-23-[(4-methylpyridin-2-yl)amino]-14,19-dioxo-4,7,10-trioxa-13,15,18-triazatricosanan-1-oyl}-L-alpha-aspartyl-L-prolyl-L-valinate (10.0 mg, 7.01 μmol) in dioxane/water (1:1, 2.5 mL) was added a sodium hydroxide solution (14 μl, 1 M, 14 μmol). The solution was freeze-dried. The residue was purified over preparative HPLC and then lyophilized. The residue was dissolved in in dioxane/water (1:1, 2.5 mL) and a sodium hydroxide solution (14 μl, 1 M, 14 μmol) was added. The solution was freeze-dried to give Compound 29 (9.0 mg, 100% purity, 87% yield) as a colorless foam. LC-MS (Method 2): Rₜ=1.49 min; MS (ESIpos): m/z=1425 [M−H]^{- 1}H-NMR (400 MHz, DMSO-d6) delta [ppm]: 0.84-0.97 (m, 1H), 1.16-1.36 (m, 1H), 1.44-1.65 (m, 1H), 1.85-1.96 (m, 1H), 2.00 (br d, 1H), 2.08 (s, 1H), 2.10-2.30 (m, 1H), 2.33-2.47 (m, 1H), 2.53-2.56 (m, 6H), 2.57-2.71 (m, 1H), 3.04-3.28 (m, 2H), 3.43-3.70 (m, 3H), 3.79-3.94 (m, 1H), 4.07-4.13 (m, 1H), 4.64-5.21 (m, 1H), 5.32 (s, 1H), 5.44-5.49 (m, 1H), 6.24 (d, 1H), 6.30 (s, 1H), 6.65 (s, 1H), 7.29 (d, 1H), 7.32-7.35 (m, 1H), 7.69-7.77 (m, 1H), 8.20-8.29 (m, 1H).

### Example 30: Preparation of disodium 3-[(N-{(2R,3R)-3-[(2S)-1-{(3R,4S,5S)-4-[(N,N-dimethyl-L-valyl-L-valyl}(methyl)amino]-3-methoxy-5-methylheptanoyl)pyrrolidin-2-yl]-3-methoxy-2-methylpropanoyl}-L-phenylalanyl)amino]propyl 1-[(2S)-2-(carboxylatomethyl)-17-{4-[({(1R)-2-carboxylato-1-[3-({3-[(propylcarbamoyl)amino]benzene-1-sulfonyl}amino)phenyl]ethyl}carbamoyl)amino]anilino}-4,17-dioxo-7,10,13-trioxa-3,16-diazaheptadecanan-1-oyl]-L-prolyl-L-valinate (Compound 30)

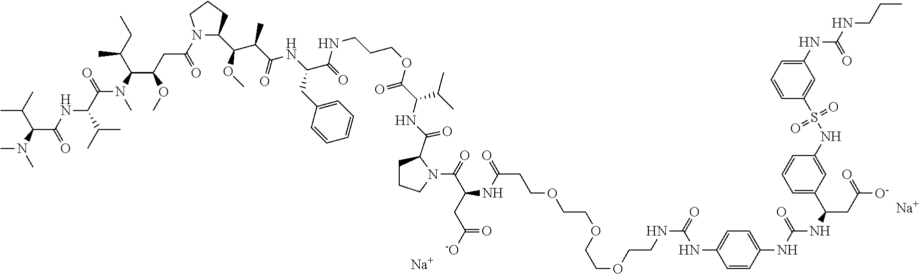

Step 1: To a solution of 3-[(N-{(2R,3R)-3-[(2S)-1-{(3R,4S,5S)-4-[(N,N-dimethyl-L-valyl-L-valyl}(methyl)amino]-3-methoxy-5-methylheptanoyl)pyrrolidin-2-yl]-3-methoxy-2-methylpropanoyl)-L-phenylalanyl)amino]propyl N-(3-(2-[2-(2-aminoethoxy)ethoxy]ethoxy}propanoyl)-L-alpha-aspartyl-L-prolyl-L-valinate.trifluoroacetic acid (1/2) (23.5 mg, 92% purity, 14.0 μmol) (Intermediate 81) in DMF (1.2 ml), were added (3R)-3-{[(4-{[(4-nitrophenoxy)carbonyl]amino}phenyl)carbamoyl]amino}-3-[3-({3-[(propylcarbamoyl)amino]benzene-1-sulfonyl}amino)phenyl]propanoic acid (10.1 mg, 14.0 μmol) (Intermediate 2) and DIEA (10.1 mg, 14.0 μmol). The mixture was stirred at rt for 1 h and then concentrated under reduced pressure. The residue was purified over preparative HPLC and freeze dried to afford 3-[(N-{(2R,3R)-3-[(2S)-1-({3R,4S,5S)-4-[(N,N-dimethyl-L-valyl-L-valyl}(methyl)amino]-3-methoxy-5-methylheptanoyl}pyrrolidin-2-yl]-3-methoxy-2-methylpropanoyl}-L-phenylalanyl)amino]propyl N-(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl}-L-alpha-aspartyl-L-prolyl-L-valinate (24 mg, 99% purity, 89% yield) as an amorphous residue. LC-MS (Method 3): Rₜ=3.52 min; MS (ESIpos): m/z=1899 [M+H]⁺.

Step 2: To a solution of 3-[(N-({2R,3R)-3-[(2S)-1-({3R,4S,5S)-4-[(N,N-dimethyl-L-valyl-L-valyl}(methyl)amino]-3-methoxy-5-methylheptanoyl)pyrrolidin-2-yl]-3-methoxy-2-methylpropanoyl)-L-phenylalanyl)amino]propyl N-(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl}-L-alpha-aspartyl-L-prolyl-L-valinate (23.0 mg, 99% purity, 12.0 μmol) in dioxane/water (1:1, 4 mL) was added a sodium hydroxide solution (120 μl, 0.1 M, 12 μmol). The solution was freeze-dried to give Compound 30 (23 mg, 100% purity, 99% yield) as a colorless foam. LC-MS (Method 3): Rₜ=3.64 min; MS (ESIpos): m/z=1897 [M+H]⁺. ¹H-NMR (600 MHz, DMSO-d6) delta [ppm]: 0.03 (br s, 1H), 0.75-0.77 (m, 1H), 0.83-0.91 (m, 10H), 0.91-0.98 (m, 7H), 1.02-1.07 (m, 1H), 1.26-1.34 (m, 1H), 1.39-1.53 (m, 2H), 1.66-1.71 (m, 1H), 1.73-1.88 (m, 3H), 1.95-2.09 (m, 2H), 2.13-2.23 (m, 1H), 2.24-2.38 (m, 3H), 2.44-2.46 (m, 1H), 2.57-2.61 (m, 1H), 2.62-2.69 (m, 2H), 2.71-2.82 (m, 4H), 2.99-3.12 (m, 4H), 3.14-3.29 (m, 8H), 3.40 (br s, 1H), 3.43 (br t, 1H), 3.45-3.63 (m, 12H), 3.76-3.82 (m, 1H), 3.93-4.05 (m, 3H), 4.08-4.12 (m, 1H), 4.42 (br d, 1H), 4.55-4.70 (m, 1H), 4.81-4.86 (m, 1H), 4.97-5.02 (m, 1H), 6.05-6.07 (m, 1H), 6.20-6.24 (m, 1H), 6.61-6.65 (m, 1H), 6.89-6.92 (m, 1H), 6.98 (br d, 1H), 7.12-7.18 (m, 2H), 7.19-7.29 (m, 6H), 7.40 (br d, 1H), 7.93-8.00 (m, 1H), 8.04-8.10 (m, 1H), 8.27-8.31 (m, 1H), 8.33 (br s, 1H), 8.35 (br s, 1H), 8.75-8.78 (m, 1H), 10.26 (s, 1H), 12.00-12.03 (m, 1H), 12.25-12.35 (m, 1H).

### Example 31: Preparation of disodium (16S)-16-[(2S)-2-{[(3R,4R,7S,20S)-7-benzyl-3-{(2S)-1-[(3R,4S,5S)-4-{[(2S)-2-{[(2S)-2-(dimethylamino)-3-methylbutanoyl]amino}-3-methylbutanoyl](methyl)amino}-3-methoxy-5-methylheptanoyl]pyrrolidin-2-yl}-4,15,15,21-tetramethyl-5,8,14,19-tetraoxo-2,13-dioxa-6,9,18-triazadocosan-20-yl]carbamoyl}pyrrolidine-1-carbonyl]-1-{4-[({(1R)-2-carboxylato-1-[3-({3-[(propylcarbamoyl)amino]benzene-1-sulfonyl}amino)phenyl]ethyl}carbamoyl)amino]anilino}-1,14-dioxo-5,8,11-trioxa-2,15-diazaoctadecan-18-oate (Compound 31)

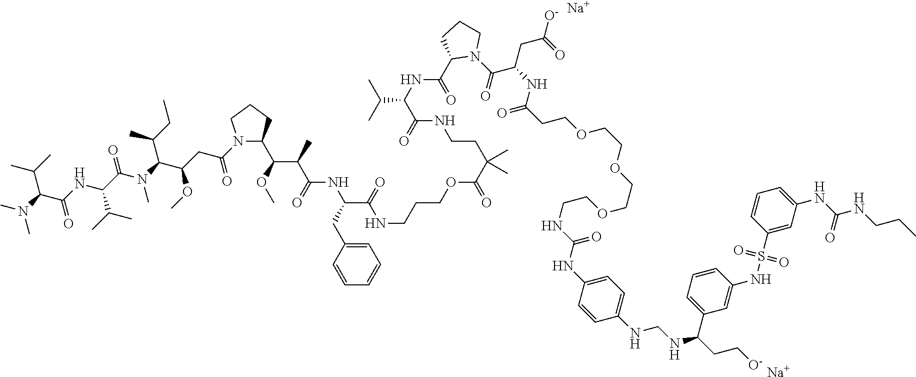 [SMILES: [Na+] ; CCCNC(=O)Nc1cccc(S(=O)(=O)Nc2cccc([C@@H](CC[O-])NCNc3ccc(NC(=O)NCCOCCOCCOCCC(=O)N[C@@H](CC(=O)[O-])C(=O)N4CCC[C@H]4C(=O)N[C@H](C(=O)NCCC(C)(C)C(=O)OCCCNC(=O)[C@H](Cc4ccccc4)NC(=O)[C@H](C)[C@@H](OC)[C@@H]4CCCN4C(=O)C[C@@H](OC)[C@H]([C@@H](C)CC)N(C)C(=O)[C@@H](NC(=O)[C@H](C(C)C)N(C)C)C(C)C)C(C)C)cc3)c2)c1 ; [Na+]]

Step 1: To a solution of N-(3-{2-[2-(2-aminoethoxy)ethoxy]ethoxy}propanoyl)-L-alpha-aspartyl-L-prolyl-N-(4-{3-[(N-{(2R,3R)-3-[(2S)-1-{(3R,4S,5S)-4-[(N,N-dimethyl-L-valyl-L-valyl}(methyl)amino]-3-methoxy-5-methylheptanoyl}pyrrolidin-2-yl]-3-methoxy-2-methylpropanoyl}-L-phenylalanyl)amino]propoxy}-3,3-dimethyl-4-oxobutyl)-L-valinamide.trifluoroacetic acid (1/1) (24.0 mg, 94% purity, 14.6 μmol) (Intermediate 85) in DMF (1.0 ml), were added (3R)-3-{[(4-{[4-nitrophenoxy)carbonyl]amino}phenyl)carbamoyl]amino}-3-[3-({3-[(propylcarbamoyl)amino]benzene-1-sulfonyl}amino)phenyl]propanoic acid (10.5 mg, 14.6 μmol) (Intermediate 2) and DIEA (25 μl, 150 μmol). The mixture was stirred at rt for 2 h and then concentrated under reduced pressure. The residue was purified over preparative HPLC and freeze dried to afford N-(14-{4-[({((1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)-L-alpha-aspartyl-L-prolyl-N-(4-{3-[(N-{(2R,3R)-3-[(2S)-1-{(3R,4S,5S)-4-[(N,N-dimethyl-L-valyl-L-valyl}(methyl)amino]-3-methoxy-5-methylheptanoyl}pyrrolidin-2-yl]-3-methoxy-2-methylpropanoyl}-L-phenylalanyl)amino]propoxy}-3,3-dimethyl-4-oxobutyl)-L-valinamide (25 mg, 100% purity, 85% yield) as a colorless foam. LC-MS (Method 3): Rₜ=3.7 min; MS (ESIpos): m/z=1006 [M+2H]²⁺.

Step 2: To a solution of N-(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)-L-alpha-aspartyl-L-prolyl-N-(4-{3-[(N-{(2R,3R)-3-[(2S)-1-{(3R,4S,5S)-4-[(N,N-dimethyl-L-valyl-L-valyl}(methyl)amino]-3-methoxy-5-methylheptanoyl}pyrrolidin-2-yl]-3-methoxy-2-methylpropanoyl}-L-phenylalanyl)amino]propoxy}-3,3-dimethyl-4-oxobutyl)-L-valinamide (23.0 mg, 11.4 μmol) in dioxane/water (1:1, 4 mL) was added a sodium hydroxide solution (23 μl, 1 M, 23 μmol). The solution was freeze-dried to give Compound 30 (15 mg, 99% purity, 63% yield) as a colorless foam. LC-MS (Method 3): Rₜ=3.69 min; MS (ESIpos): m/z=1007 [M+2H]²⁺. ¹H-NMR (500 MHz, DMSO-d6) delta [ppm]: 0.02-0.22 (m, 2H), 0.70-0.76 (m, 3H), 0.78-0.98 (m, 15H), 1.00-1.04 (m, 1H), 1.10 (d, 4H), 1.15-1.21 (m, 1H), 1.22-1.40 (m, 3H), 1.41-1.47 (m, 1H), 1.57-1.80 (m, 5H), 1.84-2.05 (m, 4H), 2.13-2.30 (m, 5H), 2.32-2.49 (m, 2H), 2.52-2.55 (m, 1H), 2.57-2.68 (m, 2H), 2.72-2.84 (m, 1H), 2.89-2.97 (m, 2H), 2.98-3.05 (m, 2H), 3.06-3.29 (m, 8H), 3.37-3.57 (m, 10H), 3.60 (br t, 1H), 3.79-4.00 (m, 4H), 4.26-4.29 (m, 1H), 4.45-4.57 (m, 1H), 4.59-4.68 (m, 1H), 4.73 (td, 1H), 4.94-4.98 (m, 1H), 6.35 (br d, 1H), 6.64 (br d, 1H), 6.93 (br d, 1H), 7.06 (t, 1H), 7.10-7.15 (m, 1H), 7.16-7.26 (m, 5H), 7.27-7.36 (m, 1H), 7.49 (s, 1H), 7.88 (s, 1H), 8.03 (br d, 1H), 8.06-8.11 (m, 1H), 8.14-8.26 (m, 1H), 8.51 (br d, 1H), 8.83 (br s, 1H), 9.66-9.96 (m, 1H), 11.20-11.33 (m, 1H).

### Example 32: Preparation of disodium 4-[(N-{(2R,3R)-3-[(2S)-1-{(3R,4S,5S)-4-[(N,N-dimethyl-L-valyl-L-valyl}(methyl)amino]-3-methoxy-5-methylheptanoyl}pyrrolidin-2-yl]-3-methoxy-2-methylpropanoyl)-L-phenylalanyl)amino]-2-methylbutan-2-yl 1-[(2S)-2-(carboxylatomethyl)-17-{4-[({(1R)-2-carboxylato-1-[3-({3-[(propylcarbamoyl)amino]benzene-1-sulfonyl}amino)phenyl]ethyl}carbamoyl)amino]anilino}-4,17-dioxo-7,10,13-trioxa-3,16-diazaheptadecanan-1-oyl]-L-prolyl-L-valinate (Compound 32)

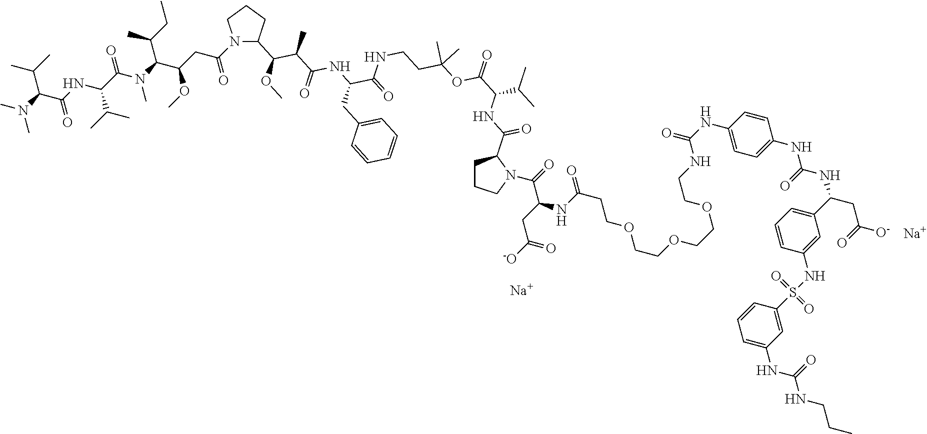

Step 1: To a solution of tert-butyl (14S)-1-amino-14-[(2S)-2-{[(3R,4R,7S,15S)-7-benzyl-3-({2S)-1-[(3R,4S,5S)-4-{[(2S)-2-{[(2S)-2-(dimethylamino)-3-methylbutanoyl]amino}-3-methylbutanoyl](methyl)amino}-3-methoxy-5-methylheptanoyl]pyrrolidin-2-yl}-4,12,12,16-tetramethyl-5,8,14-trioxo-2,13-dioxa-6,9-diazaheptadecan-15-yl]carbamoyl}pyrrolidine-1-carbonyl]-12-oxo-3,6,9-trioxa-13-azahexadecan-16-oate (14.0 mg, 83% purity, 8.27 μmol) (Intermediate 88) in DMF (0.75 ml), were added (3R)-3-{[(4-{[(4-nitrophenoxy)carbonyl]amino}phenyl)carbamoyl]amino}-3-[3-({3-[(propylcarbamoyl)amino]benzene-1-sulfonyl}amino)phenyl]propanoic acid (5.95 mg, 8.27 μmol) (Intermediate 2) and DIEA (14 μl, 83 μmol). The mixture was stirred at rt for 3 h and then concentrated under reduced pressure. The residue was purified over preparative HPLC and freeze dried to afford 3R)-3-[({4-[({(14S)-14-[(2S)-2-{[(3R,4R,7S,15S)-7-benzyl-3-((2S)-1-[(3R,4S,5S)-4-{[(2S)-2-{[(2S)-2-(dimethylamino)-3-methylbutanoyl]amino}-3-methylbutanoyl](methyl)amino}-3-methoxy-5-methylheptanoyl]pyrrolidin-2-yl}-4,12,12,16-tetramethyl-5,8,14-trioxo-2,13-dioxa-6,9-diazaheptadecan-15-yl]carbamoyl}pyrrolidine-1-carbonyl]-18,18-dimethyl-12,16-dioxo-3,6,9,17-tetraoxa-13-azanonadecan-1-yl)carbamoyl)amino]phenyl}carbamoyl)amino]-3-[3-({3-[(propylcarbamoyl)amino]benzene-1-sulfonyl}amino)phenyl]propanoic acid (12.5 mg, 100% purity, 76% yield) as a colorless foam. LC-MS (Method 3): Rₜ=4.10 min; MS (ESIpos): m/z=992 [M+2H]²⁺.

Step 2: To a solution of (3R)-3-[({4-[({(14S)-14-[(2S)-2-{[(3R,4R,7S,15S)-7-benzyl-3-((2S)-1-[(3R,4S,5S)-4-{[(2S)-2-{[(2S)-2-(dimethylamino)-3-methylbutanoyl]amino}-3-methylbutanoyl](methyl)amino}-3-methoxy-5-methylheptanoyl]pyrrolidin-2-yl}-4,12,12,16-tetramethyl-5,8,14-trioxo-2,13-dioxa-6,9-diazaheptadecan-15-yl]carbamoyl}pyrrolidine-1-carbonyl]-18,18-dimethyl-12,16-dioxo-3,6,9,17-tetraoxa-13-azanonadecan-1-yl)carbamoyl)amino]phenyl)carbamoyl)amino]-3-[3-({3-[(propylcarbamoyl)amino]benzene-1-sulfonyl)amino)phenyl]propanoic acid (11.5 mg, 5.80 μmol) in DCM (1.0 ml) was added TFA (100 μl). The mixture was stirred overnight at rt and then concentrated under reduced pressure. The residue was purified over preparative HPLC and then freeze dried to afford 4-[(N-((2R,3R)-3-[(2S)-1-({(3R,4S,5S)-4-[(N,N-dimethyl-L-valyl-L-valyl}(methyl)amino]-3-methoxy-5-methylheptanoyl)pyrrolidin-2-yl]-3-methoxy-2-methylpropanoyl}-L-phenylalanyl)amino]-2-methylbutan-2-yl N-(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl}-L-alpha-aspartyl-L-prolyl-L-valinate (7.7 mg, 100% purity, 69% yield) as a white foam. LC-MS (Method 3): Rₜ=3.72 min; MS: m/z=1927 [M+H]⁺.

Step 3: To a solution of 4-[(N-{(2R,3R)-3-[(2S)-1-((3R,4S,5S)-4-[(N,N-dimethyl-L-valyl-L-valyl}(methyl)amino]-3-methoxy-5-methylheptanoyl)pyrrolidin-2-yl]-3-methoxy-2-methylpropanoyl)-L-phenylalanyl)amino]-2-methylbutan-2-yl N-(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)-L-alpha-aspartyl-L-prolyl-L-valinate (7.70 mg, 4.00 μmol) in dioxane/water (1:1, 1.5 mL) was added a sodium hydroxide solution (8 μl, 1 M, 8 μmol). The solution was freeze-dried to give Compound 32 (7.7 mg, 100% purity, 98% yield) as a colorless foam. LC-MS (Method 3): Rₜ=3.70 min; MS (ESIpos): m/z=1927 [M+H]⁺. ¹H-NMR (400 MHz, DMSO-d6) delta [ppm]: 0.84 (s, 1H), 1.16-2.32 (m, 24H), 2.53-2.55 (m, 82H), 2.63-3.06 (m, 13H), 3.52 (s, 1H), 3.68-5.27 (m, 1H), 5.85-6.37 (m, 1H), 6.59-7.41 (m, 7H), 7.56-8.68 (m, 1H).

### Biological Evaluation

### Example B1 (Version 1): Cytotoxicity In Vitro in the Presence and Absence of Elastase

Cultivation of cells was performed according to standard procedures with the media recommended by the provider. The cells, in a total volume of 100 μL, were seeded in a 96-well plate with a white bottom (#3610). After a 24 h incubation period at 37° C. and 5% CO₂, the medium was exchanged by adding 90 μL fresh medium. The treatment started by adding the test compound in 10 μL of culture medium to the cells in triplicates. Concentrations ranging from 10⁻⁵ M to 10⁻¹³ M were chosen. Two identically treated sets of samples were prepared. One set was treated with the test compound alone, whereas to the second set the compound and 10 nM elastase (NE) was added followed by a 72 h incubation period at 37° C. and 5% CO₂. The proliferation was detected using the MTT assay (ATCC). At the end of the incubation period the MTT reagent was added to all samples for 4h. Lysis of the cells followed by addition of the detergent was done overnight. The formed dye was detected at 570 nm. The proliferation of untreated but otherwise identically handled cells was defined as the 100% value. The dose response curves allowed the determination of the respective IC₅₀ values.

### Example B2 (Version 2): Cytotoxicity In Vitro in the Presence and Absence of Elastase

The Cytotoxicity assay (Version 2) was performed as above described. Some changes were applied as the assays were performed in an automated fashion and the IC₅₀ values determination was changed. Here the change in metabolic activity of the cells, in percent, was calculated by normalization of the measured values to the absorbance values of medium containing wells without cells (=−100%) and the absorbance of the DMSO-treated cells (=0%). AC₅₀ values (activity concentration at −50%) were determined by means of a 4 parameter Hill-fit using GeneData Screener software. Values are summarized in Table 1.

TABLE 1

| | 786-O | HT29 | NCI-H292 | SUM149PT | 786-O | HT29 | NCI-H292 | SUM149PT |
|---|---|---|---|---|---|---|---|---|
| Compound | IC50 without neutrophil elastase | | | | IC50 with neutrophil elastase | | | |
| 1 | C | C | C | B | A | A | A | AA |
| 2 | C | C | C | C | A | A | A | AA |
| 3 | C | C | C | B | A | A | A | A |
| 4 | C | D | C | C | A | B | A | A |
| 5 | C | D | C | C | A | B | A | A |
| 7 | C | D | C | C | A | B | A | A |
| 8 | D | D | D | D | B | B | B | B |
| 9 | C | C | C | C | A | A | A | A |
| 10 | D | D | C | C | A | A | A | A |
| 11 | C | D | C | C | A | B | A | A |
| 12 | C | D | C | C | A | B | A | A |
| 13 | C | B | B | B | A | AA | AA | AA |
| 14 | C | C | B | B | A | AA | AA | AA |
| 15 | D | C | C | B | B | A | B | A |
| 16 | D | D | C | C | A | A | A | AA |
| 17 | D | D | C | C | A | A | A | AA |
| 18 | D | D | D | C | A | AA | AA | AA |
| 19 | D | D | D | D | A | A | AA | AA |
| 20 | D | D | C | nd | A | B | A | nd |
| 21 | D | D | C | nd | A | B | A | nd |
| 25 | nd | D | D | D | nd | A | B | A |
| 26 | nd | D | D | D | nd | A | A | AA |
| 27 | D | D | D | D | A | A | A | AA |
| 28 | C | D | C | C | A | A | A | A |
| 29 | C | D | C | C | A | B | A | A |
| 30 | C | C | B | B | B | A | A | A |
| 31 | C | B | B | B | B | AA | AA | AA |
| 32 | C | B | B | B | B | A | A | A |

IC₅₀ data are designated within the following ranges:
AA = 0.1-1 nM;
A = 1.0-9.9 nM;
B = 10-99 nM;
C = 100-999 nM;
D = >1000 nM

### Example B3

αᵥβ₃ Binding Assay

Binding affinity of the respective avb3 inhibitor is tested in an ELISA-like manner. 96-well plates are coated with vitronectin (1 μg/ml) or fibronectin 0.5 μg/ml) and afterwards uncoated surface space is blocked with BSA. After a 2 h pre-incubation of a serial dilution of the test compound (starting at 20 μM in a 1:5 dilution) with the extracellular domain of the integrin (2 μg/ml, αvβ3 or α5β1) the solution is added to the coated plates. After an 1h incubation at rt and several washing steps, an integrin specific antibody is added for 1 h at rt. After three washing steps the secondary peroxidase-labeled antibody is added to the plate. After an 1h incubation at RT the plate is developed by quick addition of SeramunBlau (50 μL per well, Seramun Diagnostic GmbH, Heidesee, Germany) and incubated for 5 min at rt in the dark. The reaction is stopped with 0.3M H₂SO₄ (50 μL/well), and the absorbance was measured at 450 nm with a plate reader. IC₅₀ of each compound is tested in duplicate, and the resulting inhibition curves is analyzed. As a reference standard, Cilengitide is used. The αᵥβ₃ binding of the reference is used as 100% value.

### Example B4: Stability Studies

### Rat and Human Plasma

1 mg of the respective test compound of is dissolved in a mixture of 1.5 mL dimethylsulfoxide and 1 ml water. For complete dissolution, the HPLC vial is shaken and treated with an ultrasound. 500 μl of this solution is added to 0.5 mL of plasma with vortexing at a temperature of 37° C. Aliquots (10 μL each) are taken at respective time points and analyzed by HPLC to determine the amount of the test compound. All data is given as percent area of the initial compound at to.

| | Stability rat | Stability human |
|---|---|---|
| Compound | plasma after 4 h | plasma after 4 h |
| 13 | 100% | 100% |
| 17 | 100% | 100% |
| 26 | 100% | 100% |

### Method for Measurement of Stability in Buffer:

0.15 mg of the test compound are solved in 0.1 ml dimethylsulfoxide and 0.4 ml acetonitrile. For complete dissolution the HPLC vial with the sample solution is shaken and sonicated. Then 1.0 ml of the respective buffer solution is added, and the sample is vortexed. The sample solution is analysed by HPLC to determine the amount of the test compound and up to two byproducts at a particular time over a period of 24 h at 37° C. to values result from a sample immediately taken after vortexing with buffer at RT. The peak areas (in percentage) are used for quantification.

| | Stability buffer | Stability buffer |
|---|---|---|
| Compound | pH 4 after 24 h | pH 7.4 after 24 h |
| 13 | 92% | 100% |
| 17 | 100% | 100% |
| 26 | 94% | 100% |

### Example B5: In Vitro Tests for Determining Cellular Permeability

Caco-2: The cell permeability of a substance can be investigated by means of in vitro testing in a flux assay using Caco-2 cells [M. D. Troutman and D. R. Thakker, Pharm. Res. 20 (8), 1210-1224 (2003)]. For this purpose, the cells were cultured for 15-16 days on 24-well filter plates. For the determination of permeation, the respective test substance was applied in a HEPES buffer to the cells either apically (A) or basally (B) and incubated for 2 hours. After 0 hours and after 2 hours, samples were taken from the cis and trans compartments. The samples were separated by HPLC (Agilent 1200, Böblingen, Germany) using reverse phase columns. The HPLC system was coupled via a Turbo Ion Spray Interface to a Triple Quadropol mass spectrometer API 4000 (AB SCIEX Deutschland GmbH, Darmstadt, Germany). The permeability was evaluated on the basis of a Papp value, which was calculated using the formula published by Schwab et al. [D. Schwab et al., J. Med. Chem. 46, 1716-1725 (2003)]. A substance was classified as actively transported when the ratio of Papp (B-A) to Papp (A-B) (efflux ratio) was >2 or <0.5. Compounds with an efflux ratio of 0.5-2 are considered not actively transported. In Table 2, reference compounds are provided for comparison. Ref 1 is exatecan mesylate, Ref 2 is DXd, and Ref 3 is 10,11-methylenedioxy-camptothecin.

TABLE 2
Caco-2 Transport data of test compounds.

| | Caco-2 Flux | |
|---|---|---|
| Compound | Papp A-B [nm/s] | Efflux ratio |
| 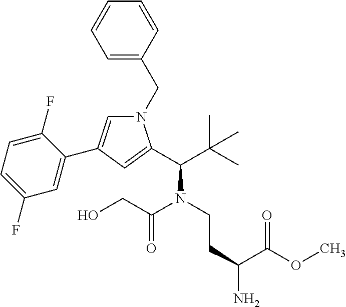 [SMILES: COC(=O)[C@@H](N)CCN(C(=O)CO)[C@@H](c1cc(-c2cc(F)ccc2F)cn1Cc1ccccc1)C(C)(C)C] | BB | A |
| 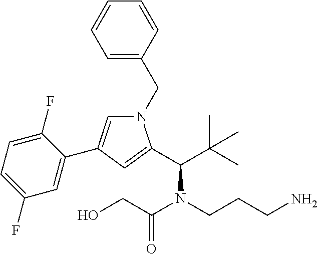 [SMILES: CC(C)(C)[C@H](c1cc(-c2cc(F)ccc2F)cn1Cc1ccccc1)N(CCCN)C(=O)CO] | AA | A |
| 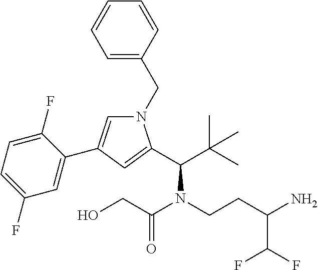 [SMILES: CC(C)(C)[C@H](c1cc(-c2cc(F)ccc2F)cn1Cc1ccccc1)N(CCC(N)C(F)F)C(=O)CO] | CC | B |
| 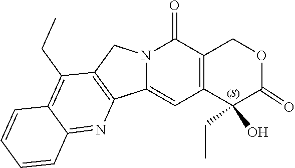 | CC | B |
| Ref 1 | CC | B |
| Ref 2 | AA | A |
| Ref 3 | CC | B |

Permeability Papp A-B:
AA = 1-9 nm/s;
BB = 10-99 nm/s;
CC = 100-999 nm/s.
A = actively transported;
B = not actively transported

P-glycoprotein (P-gp) assay: Many tumor cells express transporter proteins for drugs, and this frequently accompanies the development of resistance towards cytostatics. Substances which are not substrates of such transporter proteins, such as P-glycoprotein (P-gp) or BCRP, for example, could therefore exhibit an improved activity profile. The substrate properties of a substance for P-gp (ABCB1) are determined by means of a flux assay using LLC-PK1 cells which overexpress P-gp (L-MDR1 cells) [A. H. Schinkel et al., J. Clin. Invest. 96, 1698-1705 (1995)]. For this purpose, the LLC-PK1 cells or L-MDR1 cells are cultured on 96-well filter plates for 3-4 days. For determination of the permeation, the respective test substance, alone or in the presence of an inhibitor (such as ivermectin or verapamil, for example), is applied in a HEPES buffer to the cells either apically (A) or basally (B) and incubated for 2 hours. After 0 hours and after 2 hours, samples are taken from the cis and trans compartments. The samples are separated by HPLC using reverse phase columns. The HPLC system is coupled via a Turbo Ion Spray Interface to a Triple Quadropol mass spectrometer API 3000 (Applied Biosystems Applera, Darmstadt, Germany). The permeability is evaluated on the basis of a Papp value, which is calculated using the formula published by Schwab et al. [D. Schwab et al., J. Med. Chem. 46, 1716-1725 (2003)]. A substance is classified as P-gp substrate when the efflux ratio of Papp (B-A) to Papp (A-B) is >2.

### Example B6: Pharmacokinetics

Male rats (n=3) were given an intravenous 0.5 mg/kg bolus dose of test article dissolved in plasma with 1% DMSO and 2% ethanol. Serial blood samples were collected up to 24 hours post-dose. Plasma was harvested from blood samples and stored frozen until sample analysis. Test article plasma concentrations were analyzed by LC/MS/MS. Pharmacokinetic parameters were calculated using non-compartmental analysis.

TABLE 3
Pharmacokinetics in rat after intravenous
administration of test articles.

| Com- | IV Dose | CL plasma | V_{SS} | AUC | t_{1/2} |
|---|---|---|---|---|---|
| pound | (mg/kg) | (mL/min/kg) | (L/kg) | (mg · h/L) | (h) |
| 5 | 0.5 | B | B | C | C |
| 6 | 0.5 | C | C | D | E |
| 13 | 0.5 | B | B | C | C |
| 14 | 0.5 | A | B | A | A |
| 17 | 0.5 | B | C | C | B |
| 18 | 0.5 | A | A | A | A |
| 20 | 0.5 | B | B | C | D |
| 21 | 0.5 | B | B | C | D |
| 26 | 0.5 | B | A | C | E |
| 27 | 0.5 | A | A | A | A |
| | | wherein | wherein | wherein | wherein |
| | | A: 0.02-0.5 | A: 0.01-0.1 | A: 100-500 | A: 10-40 |
| | | B: 0.5-5 | B: 0.1-0.3 | B: 10-100 | B: 5-10 |
| | | C: >5 | C: 0.3-0.9 | C: 1-10 | C: 1-5 |
| | | | | D: <1 | D: 0.5-1 |
| | | | | | E: <0.5 |

### Example B7: In Vivo Xenotransplantation Studies

The anti-tumor activities of the test compounds were examined in a SCLC mouse xenograft model. For this purpose, immunocompromised mice were implanted subcutaneously with tumor cells or tumor fragments. At a mean tumor size of 20-40 mm2, animals were randomized, split into treatment and control groups (n=10 animals/group), and treatment started with vehicle only or example C4b or C5 (formulation: phosphate buffered saline (“PBS”); application route: intravenously into the tail vein (“i.v.”)). Intravenous treatments were performed on two consecutive days once daily followed by five days of drug holiday without any treatment. The tumor size and the body weight were determined twice weekly. The tumor area was detected by means of an electronic caliper [length (mm)×width (mm)]. The experimental groups were ended after three 3 weeks of treatment. In vivo anti-tumor efficacy is presented as T/C ratio of mean tumor area measured for treatment and control group on the last day at which the vehicle control remained in study (Treatment/Control; mean tumor area of treatment group/mean tumor area of control group. A compound having a T/C below 0.5 is defined as active (i.e., effective). Statistical analysis was assessed using GraphPadPrizm software. An unpaired t-test was performed versus the control group.

### Example B8: Cytokine Release after PBMC Stimulation with TLR7/8 Compounds

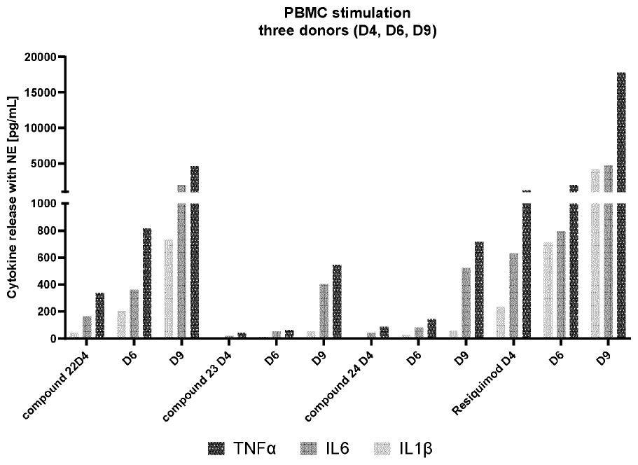

PBMC from three different donors were purified and two identical plates were prepared. 1×105 cells (PBMC) per well in a 96 MTP seeded and incubated for 1 h at 37° C., 5% C02. All test and reference compounds were added to the PBMCs in volumes of 10 μL in duplicate and incubated for 24 hours at 37° C., 5% C02. To the first plate the compounds were added after a prior incubation with human neutrophil elastase (NE). The samples on the second plate were treated with the different compounds without NE treatment. After the incubation period, plates were centrifuged at 200×g for 10 min. Cell culture supernatants were collected and stored at −80° C. until needed for analysis. Cytokine levels were determined using Luminex methodology per the manufacturer's protocol using the Human Cytokine/Chemokine Magnetic bead panel from Millipore Sigma with standards range 3.2, 16, 80, 400, 2000, and 10,000 pg/mL. Relative fluorescence units were measured to allow calculation of cell viability as percentage of control (POC) values, the results of which are illustrated in FIG. 1.

### Example B9: Elastase Cleavability Test

The test compound was added to buffer (150 mM NaCl, 10 mM CaCl₂, 0.05% BSA) to a final concentration of 5 μM (0.5% DMSO). The reaction was started by adding different concentrations of human Elastase (0, 20, 40, 60 nM) to the reaction vials. After incubation for 1 h at 37° C., the enzymatic reaction was stopped by precipitation in 50% ACN. Subsequently, the samples were subjected to HPLC-MS analysis to determine the concentration of test compound and of its metabolite.

TABLE 3
Elastase cleavability results.

| | Elastase Concentration | |
|---|---|---|
| Compound | (nM) | Payload release after 1 h |
| 13 | 0 | D |
| 13 | 20 | A |
| 17 | 0 | D |
| 17 | 20 | C |
| 26 | 0 | D |
| 26 | 20 | B |

wherein A = >70%; B = >20%; C = >1%; D = <1%.

1-74. (canceled)

75. A compound or a pharmaceutically acceptable salt thereof, having the structure of Formula (II), Formula (III), Formula (IV), Formula (V), or Formula (VII): 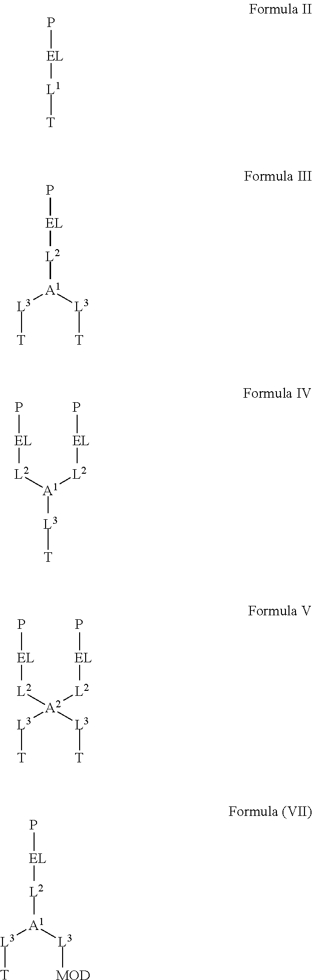
wherein:
each T is a prostate-specific membrane antigen binder, carbonic anhydrase 9 binder, fibroblast activation protein binder, folate receptor binder, heat-shock protein 90 binder, or an αvβ6 integrin binder, preferably 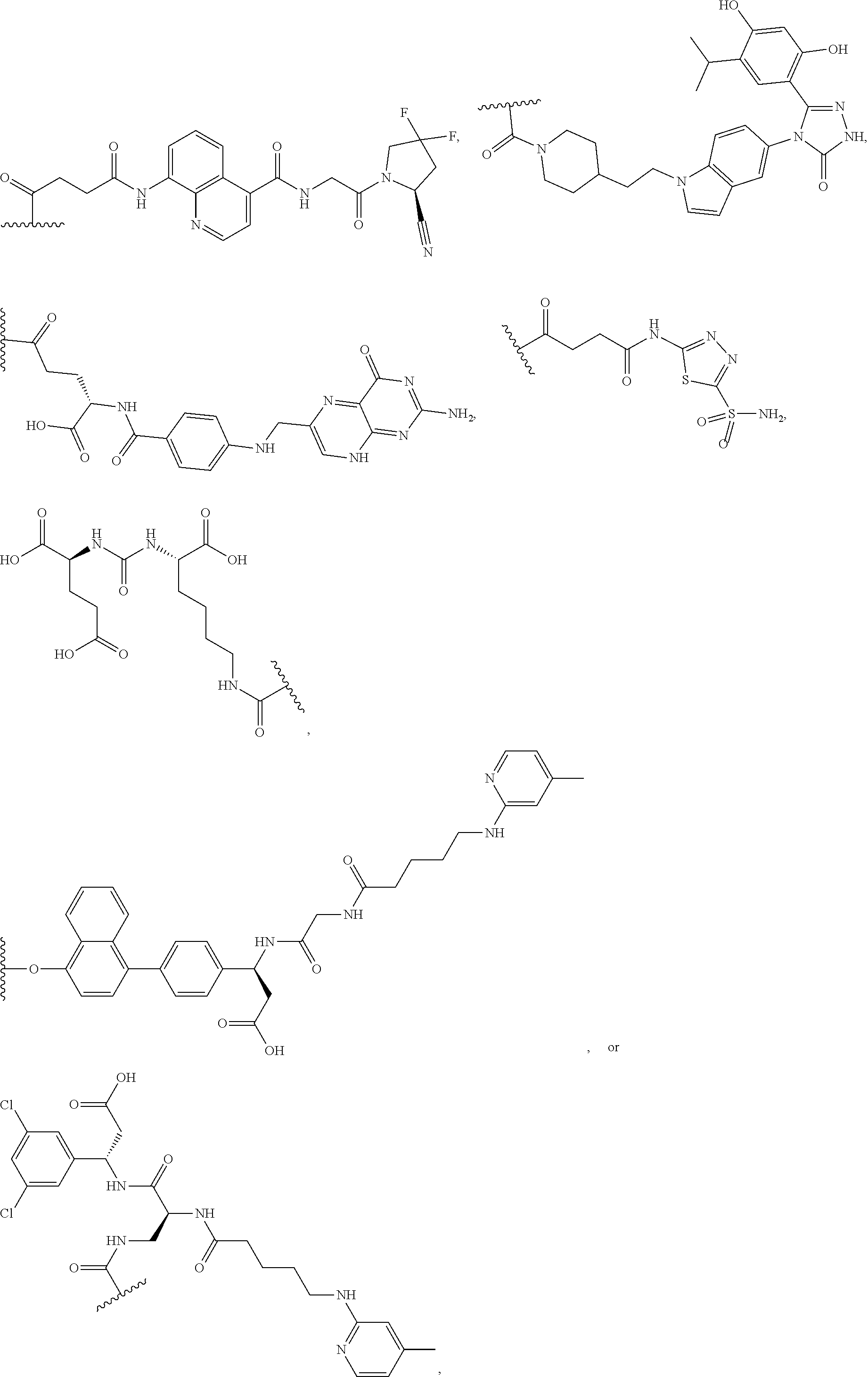
each L¹, L², and L³, is independently a bivalent linker,
A¹ is a trivalent linker,
A² is a tetravalent linker;
each EL is a cleavable peptide linker, preferably cleaved by cathepsin B, legumain, or neutrophil elastase, more preferably neutrophil elastase, and
each P is a payload,
MOD is a pharmacokinetic modulating group
preferably having the structure of Formula (II) or Formula (III): 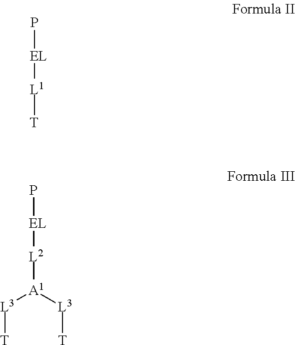
preferably having the structure: 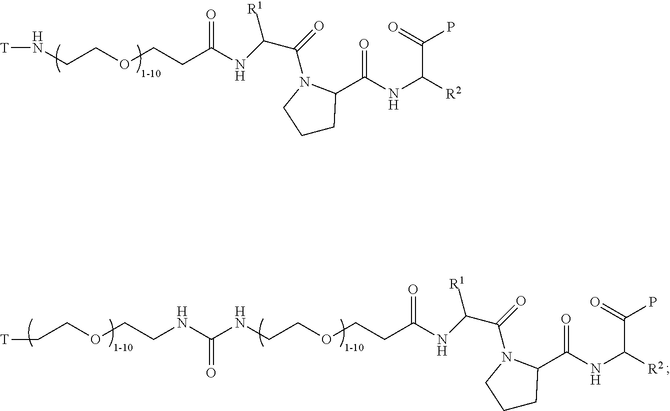
wherein:
R¹ is hydrogen, —CH₃, —CH₂CH₃, —CH₂CH₂CH₃, —CH₂C(O)NH₂, or —CH₂C(O)OH; and
R² is —CH₃, —CH(CH₃)₂, —CH₂CH(CH₃)₂, or —CH(CH₃)CH₂CH₃.
preferably having the structure:

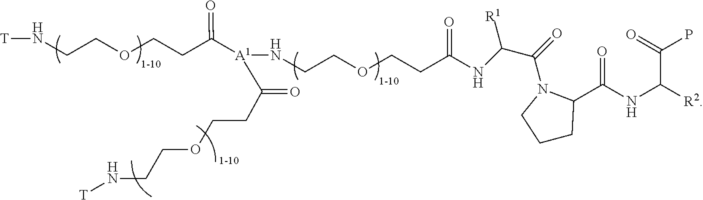

76. The compound of claim 75, or a pharmaceutically acceptable salt thereof, wherein P is a tubulin polymerization inhibitor, topoisomerase inhibitor, oxidative phosphorylation inhibitor, kinase inhibitor, dihydrofolate reductase inhibitor, histone deacetylase inhibitor, microtubule inhibitor, or an immuonomodulator, preferably a camptothecin or camptothecin derivative, an auristatin or auristatin derivative, a kinesin spindle protein inhibitor, a cyclin-dependent kinase 9 inhibitor, a toll-like receptor agonist, an epidermal growth factor receptor inhibitor, or a taxane, more preferably independently selected from the group consisting of: 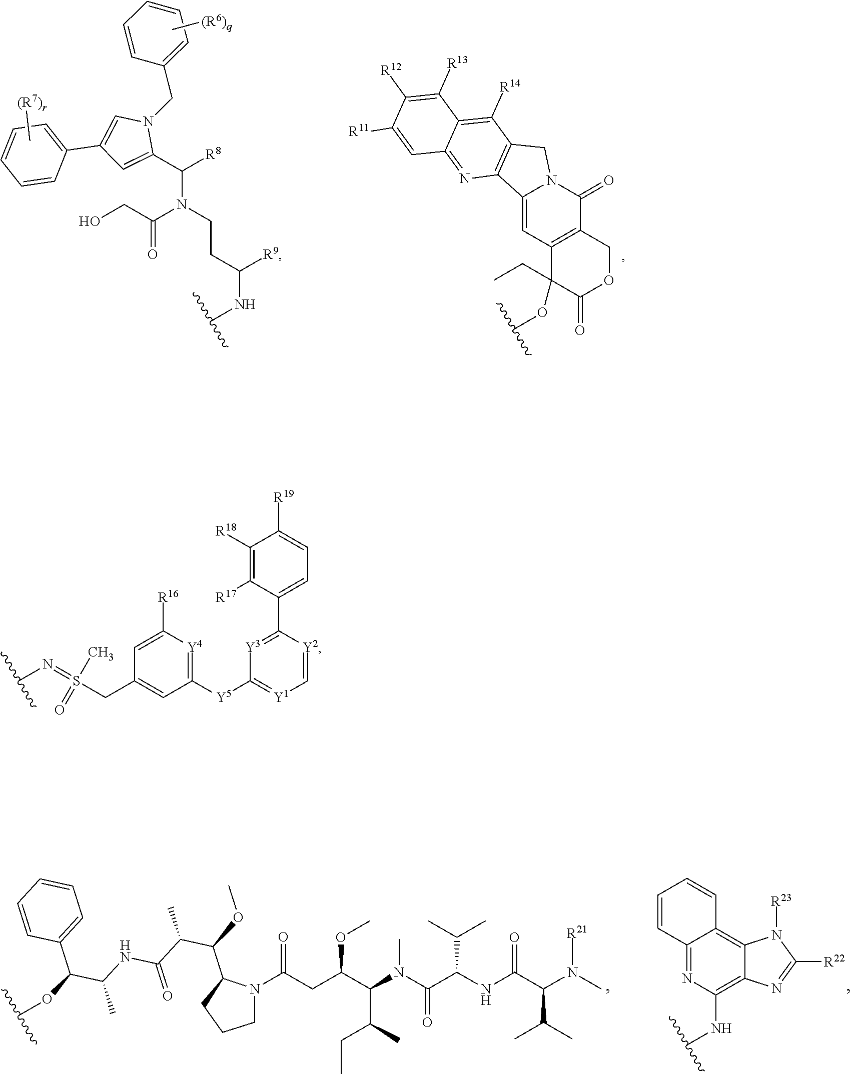 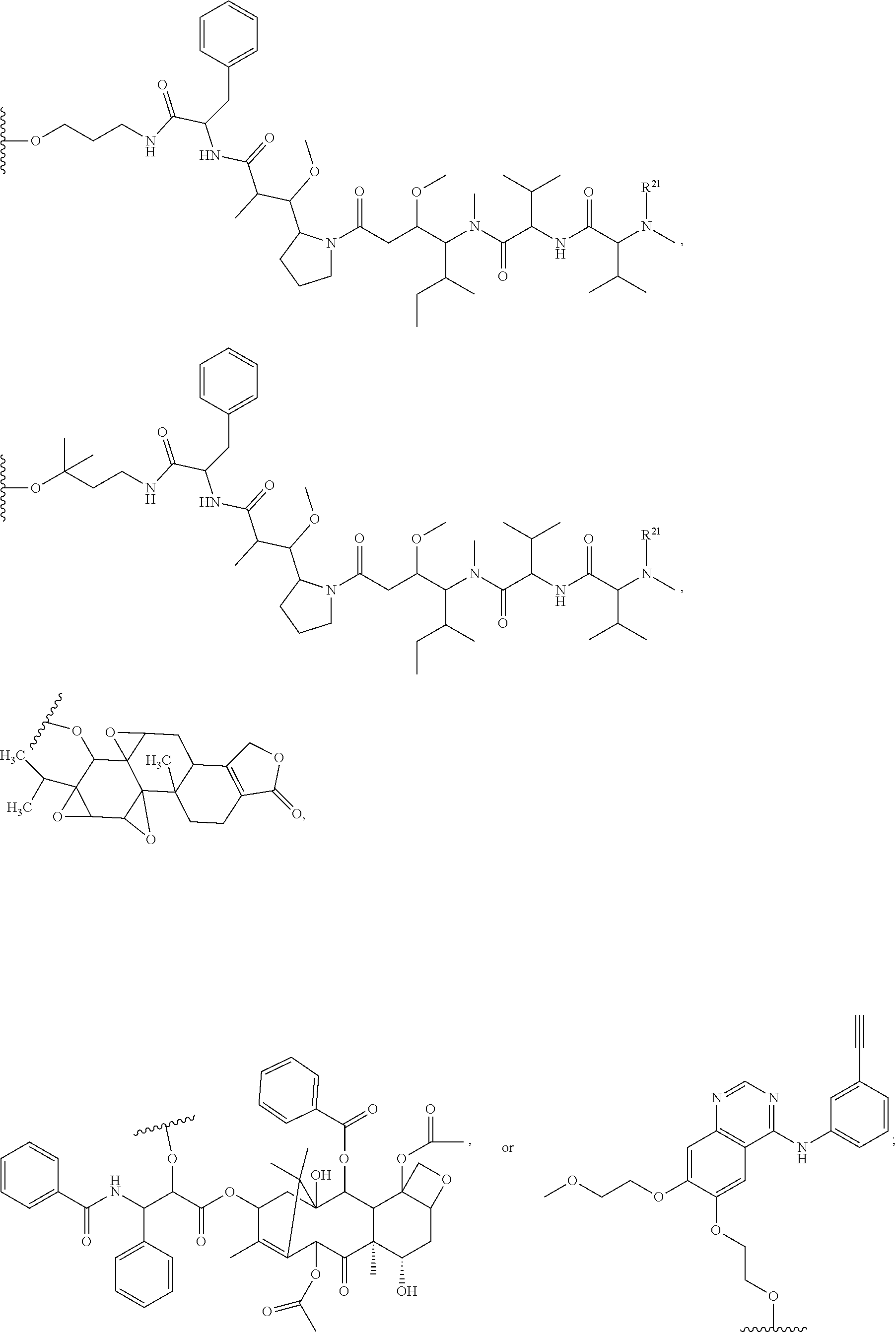
or a pharmaceutically acceptable salt thereof, wherein:
each R⁶ and R⁷ is independently hydrogen, halogen, CN, —C₁₋₆ alkyl, or C₁₋₆ haloalkyl;
R⁸ is hydrogen, halogen, CN, —C₁₋₆ alkyl, C₁₋₆ haloalkyl, C₃₋₆ cycloalkyl, or 5- to 7-membered heterocycloalkyl;
R⁹ is hydrogen, halogen, CN, C₁₋₆ alkyl, —C(O)NH₂, —C(O)NHC₁₋₆ alkyl, —C(O)N(C₁₋₆ alkyl)₂, —C(O)NHC₁₋₆ alkyl-C(O)NHC₁₋₆ alkyl, —C(O)NHC₁₋₆ alkyl-NHC(O)C₁₋₆ alkyl, —NH₂, —NHC₁₋₆ alkyl, —N(C₁₋₆ alkyl)₂, —NHC(O)C₁₋₆ alkyl, —OH, or —OC₁₋₆ alkyl; wherein each C₁₋₆ alkyl is substituted with 0-5 R¹⁰;
R¹⁰ is in each instance independently selected from the group consisting of hydrogen, halogen, CN, —COOH, —CONH₂, —NH₂, —NHCH₃, —N(CH₃)₂, —OH, and —OCH₃;
R¹¹ and R¹² are each independently hydrogen, halogen, C₁₋₆ alkyl, C₁₋₆ haloalkyl, or —OH; or R¹¹ and R¹², taken together, form a 5- or 6-membered heterocycle;
R¹³ and R¹⁴ are each independently hydrogen, C₁₋₆ alkyl, or C₁₋₆ alkylamine; or R¹³ and R¹⁴, taken together, form a C6 carbocycle substituted with —N(R¹⁵)₂;
each R¹⁵ is independently hydrogen, C₁₋₆ alkyl, —C(O)C₁₋₆ alkyl, —C(O)NHC₁₋₆ alkyl or —C(O)OC₁₋₆ alkyl; wherein the C₁₋₆ alkyl of R⁵ is optionally substituted with halogen, hydroxy, phenyl, or heteroaryl; or R⁵ is a cleavable prodrug group;
R¹⁶ is hydrogen, halogen, C₁₋₆ alkyl, C₁₋₆ haloalkyl, —OH, —OCH₃, or —OCF₃;
R¹⁷ is hydrogen, halogen, C₁₋₆ alkyl, C₁₋₆ haloalkyl, —OH, —OCH₃, or —OCF₃; or R¹⁶ and R¹⁷, taken together, form a heteroalkylene group of the formula: —O—C₂₋₁₀ alkylene-O—, —NH—C₂₋₁₀ alkylene-O—, or —NH—C₂₋₁₀ alkylene-NH—; wherein the heteroalkylene group is optionally substituted with R²;
R¹⁸ is hydrogen, halogen, C₁₋₆ alkyl, C₁₋₆ haloalkyl, —OH, —OCH₃, or —OCF₃; or R¹⁶ and R¹⁸, taken together, form a heteroalkylene group of the formula: —O—C₂₋₁₀ alkylene-O—, —NH—C₂₋₁₀ alkylene-O—, or —NH—C₂₋₁₀ alkylene-NH—; wherein the heteroalkylene group is optionally substituted with R²;
R¹⁹ is hydrogen, halogen, C₁₋₆ alkyl, C₁₋₆ haloalkyl, —OH, —OCH₃, or —OCF₃;
R²⁰ is hydrogen, halogen, C₁₋₆ alkyl, C₁₋₆ haloalkyl, or —OH;
R²¹ is hydrogen or C₁₋₆ alkyl;
R²² is hydrogen or C₁₋₆ alkyl, wherein the C₁₋₆ alkyl is unsubstituted or substituted with R²⁴;
R²³ is hydrogen, C₁₋₆ alkyl, or benzyl, wherein the C₁₋₆ alkyl or benzyl is unsubstituted or substituted with one, two, or three R²⁵ groups;
R²⁴ is —OH, —O(C₁₋₆ alkyl), —NH₂, —NH(C₁₋₆ alkyl), —N(C₁₋₆ alkyl)₂, —SH, or —S(C₁₋₆ alkyl);
R²⁵ is —OH, —O(C₁₋₆ alkyl), —NH₂, —NH(C₁₋₆ alkyl), —N(C₁₋₆ alkyl)₂, —NHS(O)₂(C₁₋₆ alkyl), C₁₋₆ alkyl, C₁₋₆ aminoalkyl, or OCH₂CH₂NHC(O)(C₁₋₆ aminoalkyl);
each Y¹, Y², Y³, and Y⁴ is independently —CH, —CF, or N;
Y⁵ is CH₂, NH, or O;
q is 0, 1, 2, 3, 4, or 5; and
r is 0, 1, 2, 3, 4, or 5.
even more preferably, 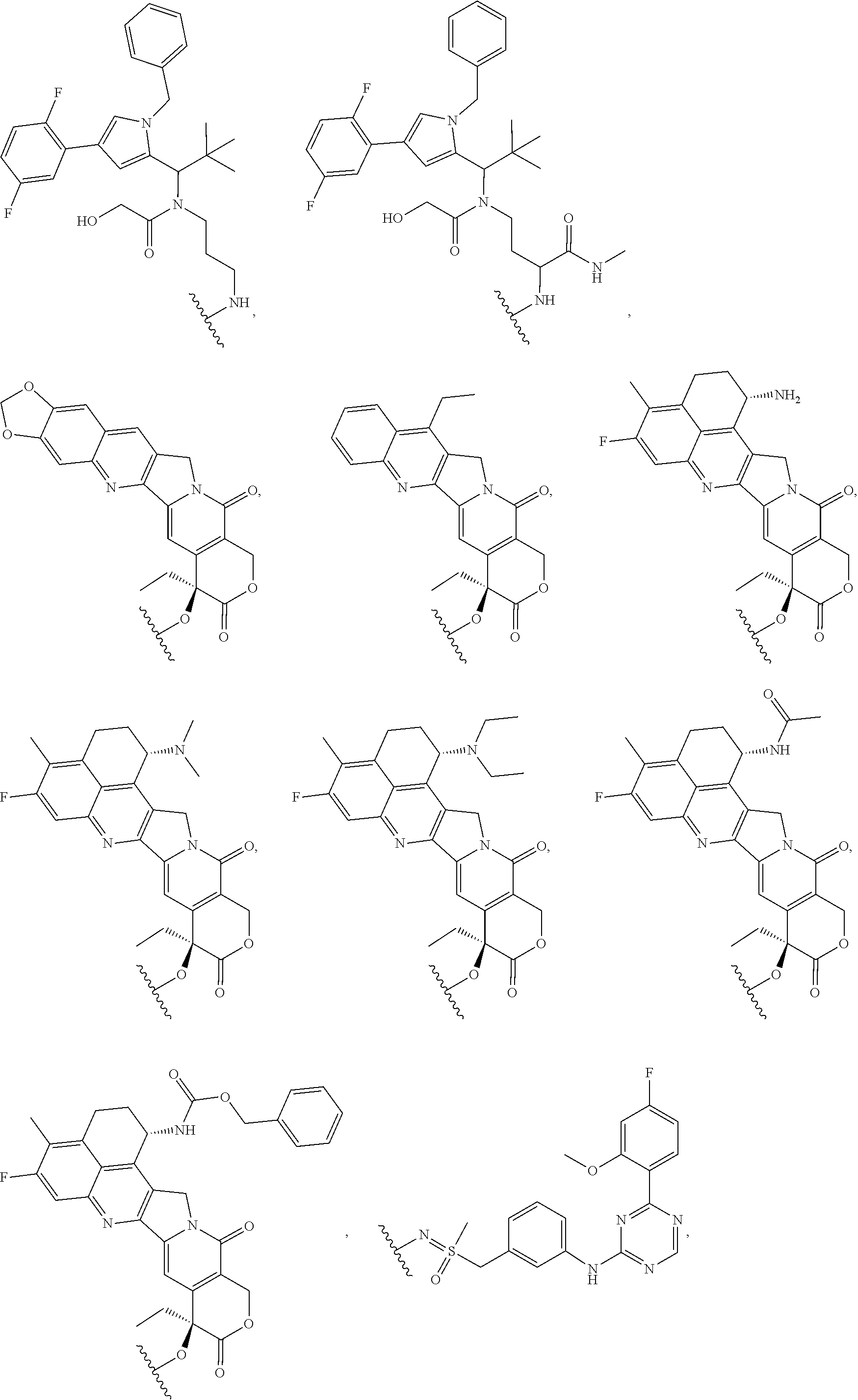 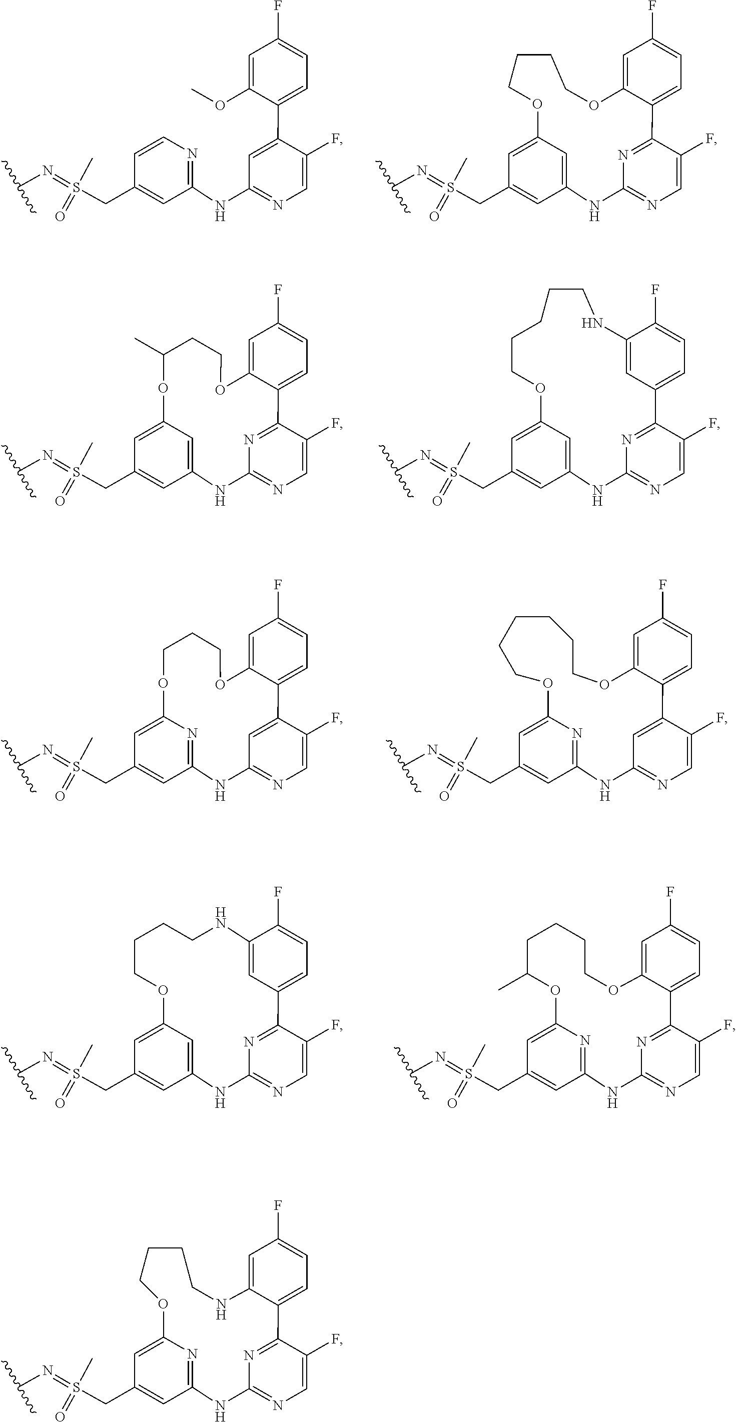 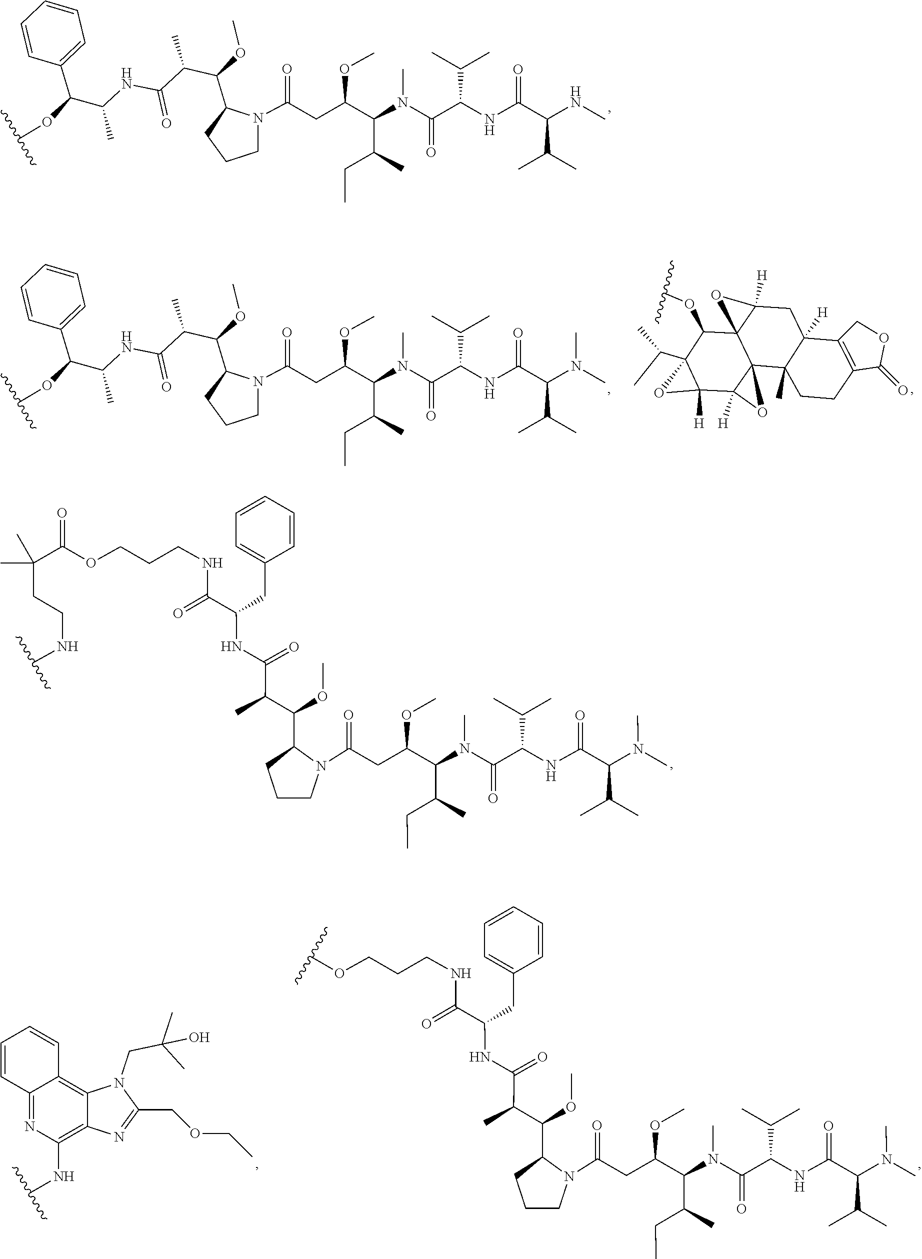 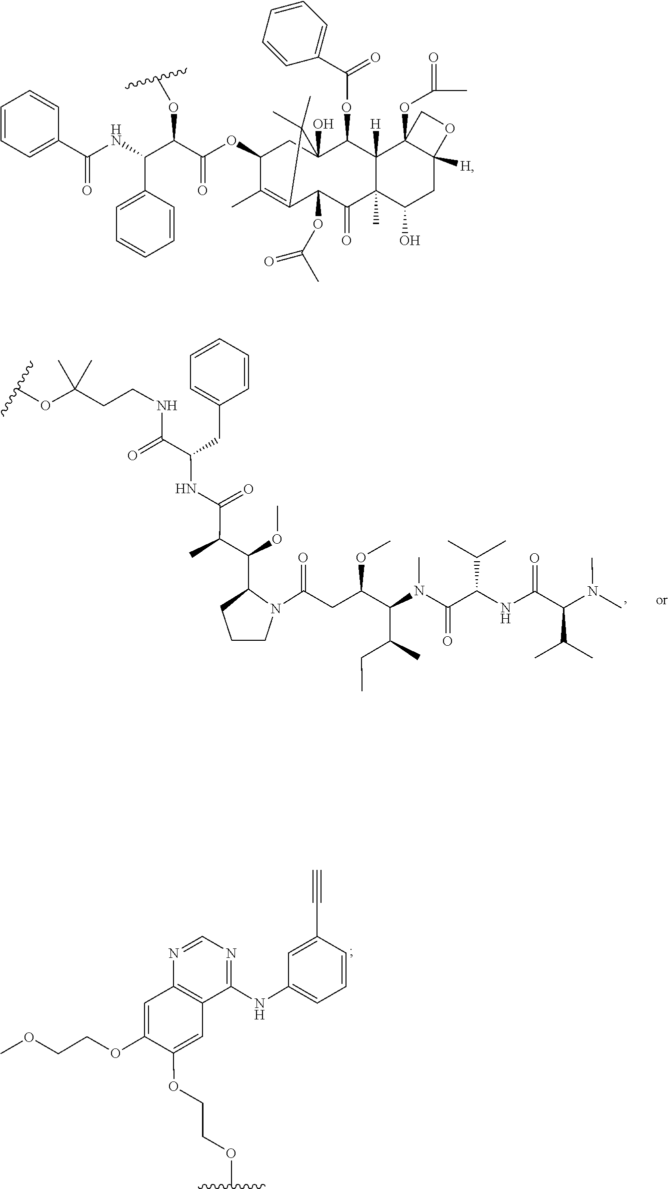
or a pharmaceutically acceptable salt thereof.
even more preferably 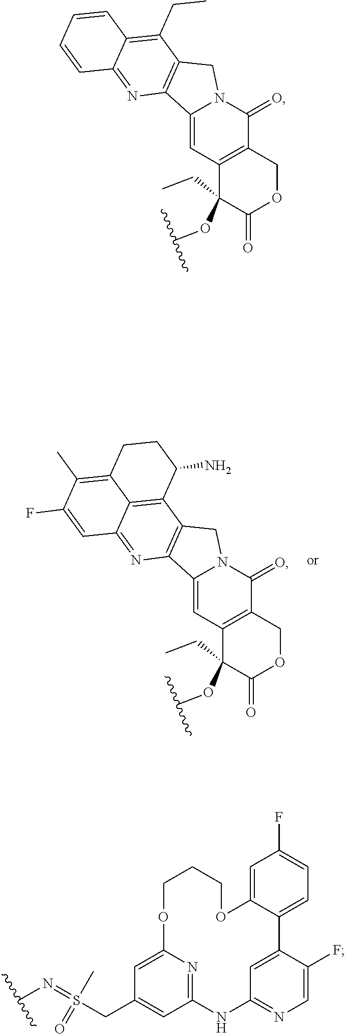 [SMILES: *O[C@]1(CC)C(=O)OCc2c1cc1n(c2=O)Cc2c-1nc1ccccc1c2CC ; *O[C@]1(CC)C(=O)OCc2c1cc1n(c2=O)Cc2c-1nc1cc(F)c(C)c3c1c2[C@@H](N)CC3 ; *N=S(C)(=O)Cc1cc2nc(c1)OCCCOc1cc(F)ccc1-c1cc(ncc1F)N2]
or a pharmaceutically acceptable salt thereof.

77. The compound of claim 75, or a pharmaceutically acceptable salt thereof, having the structure: 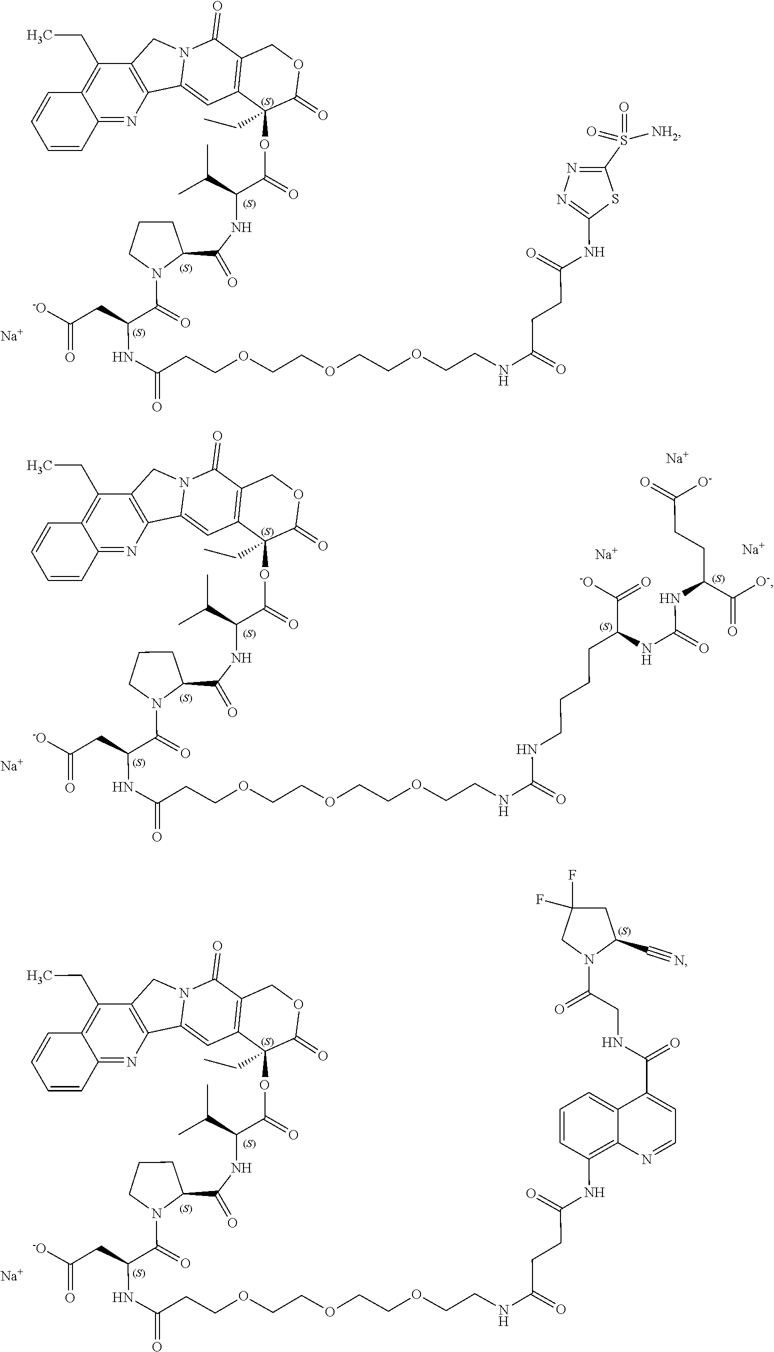 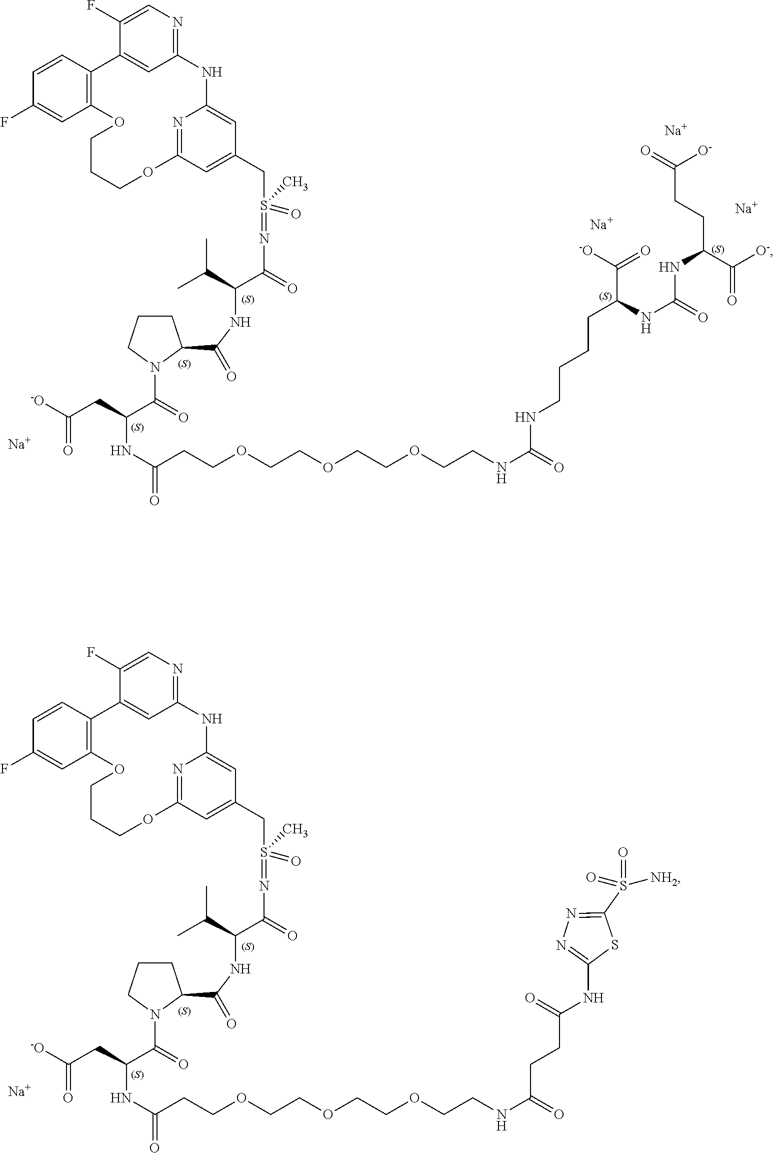 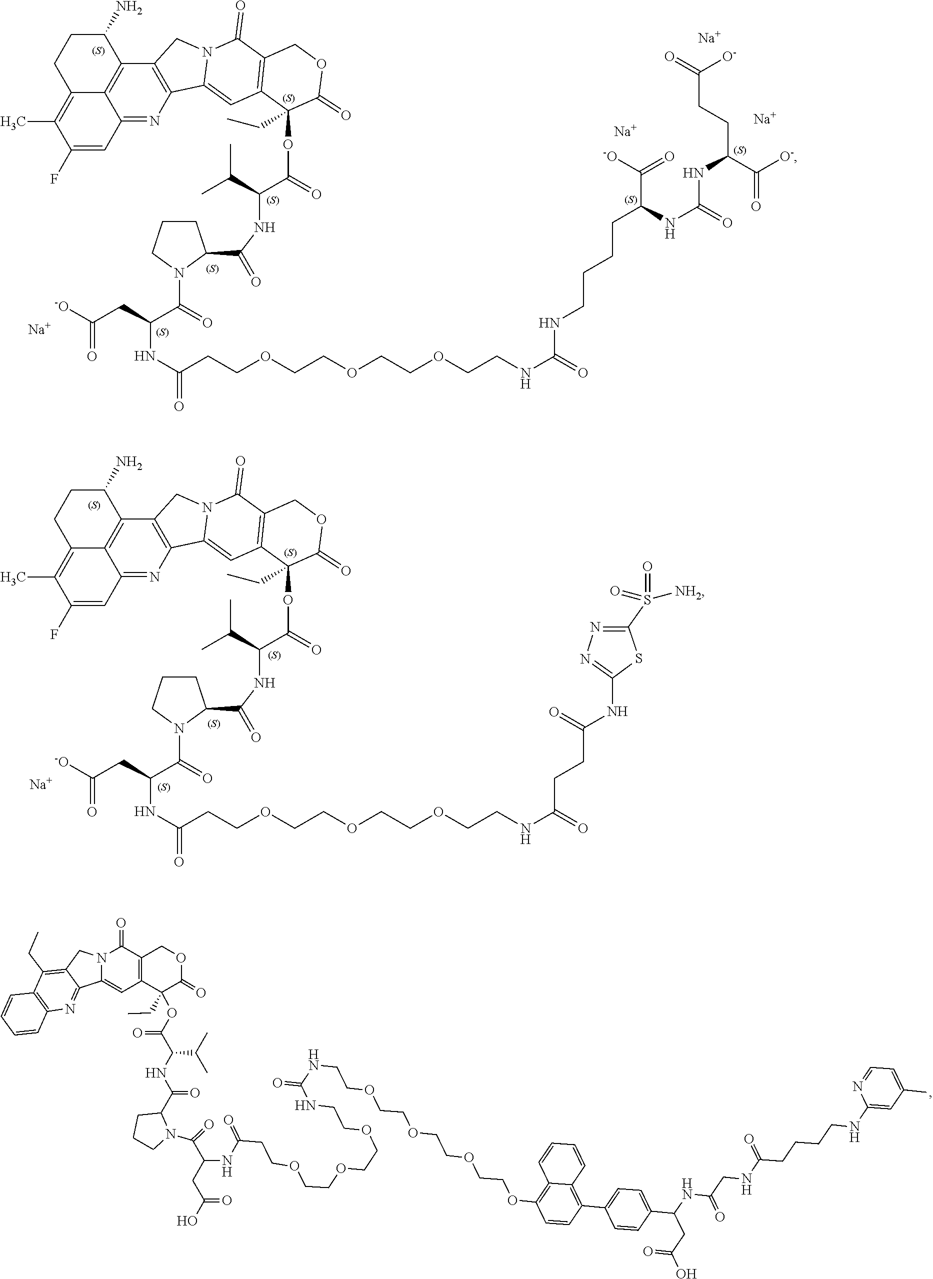 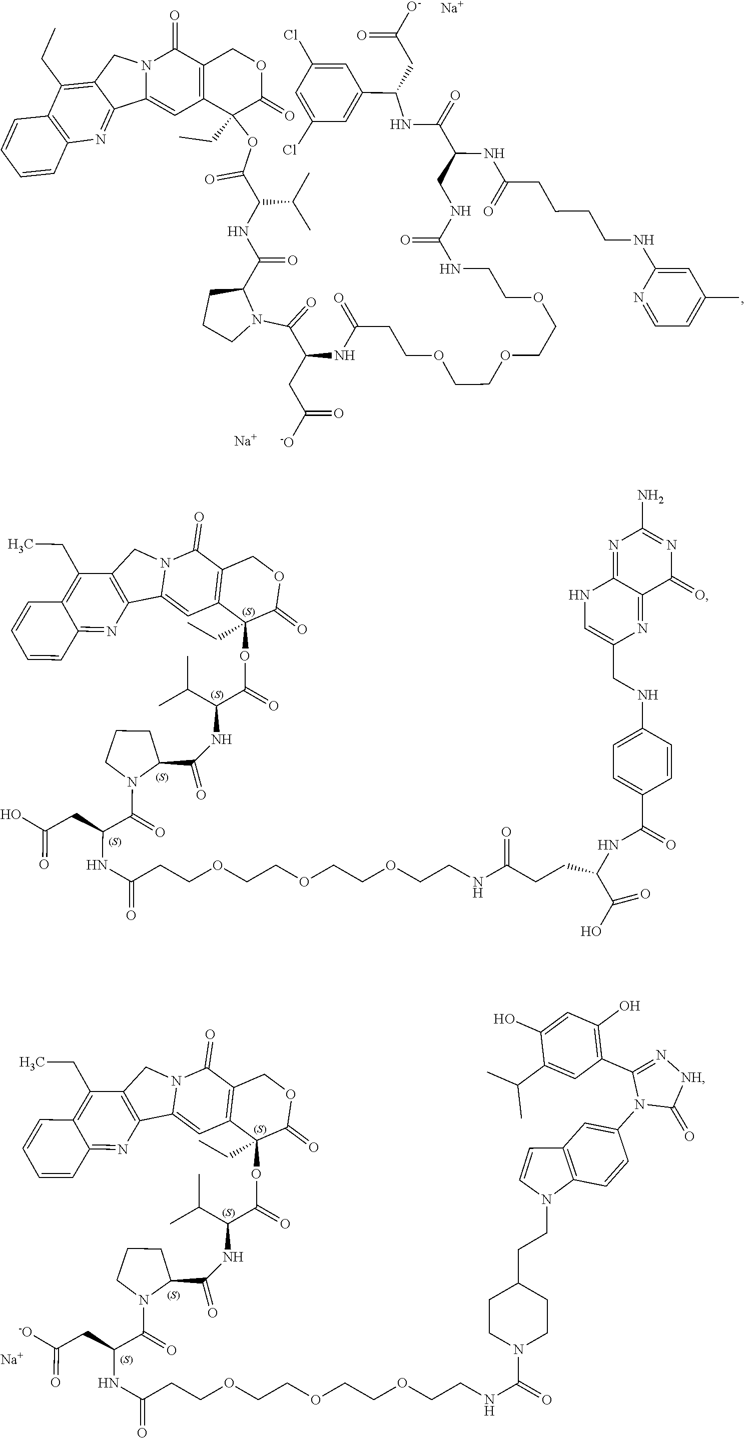 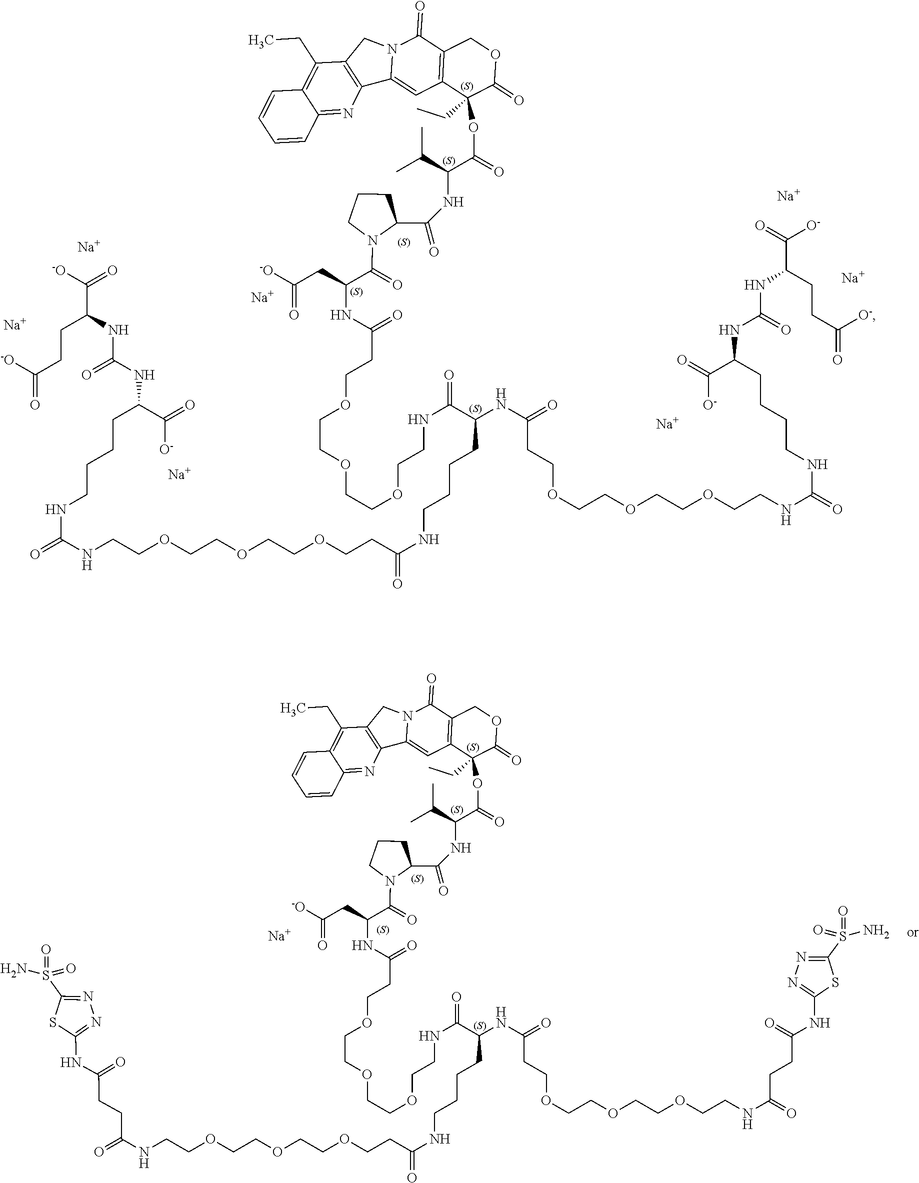 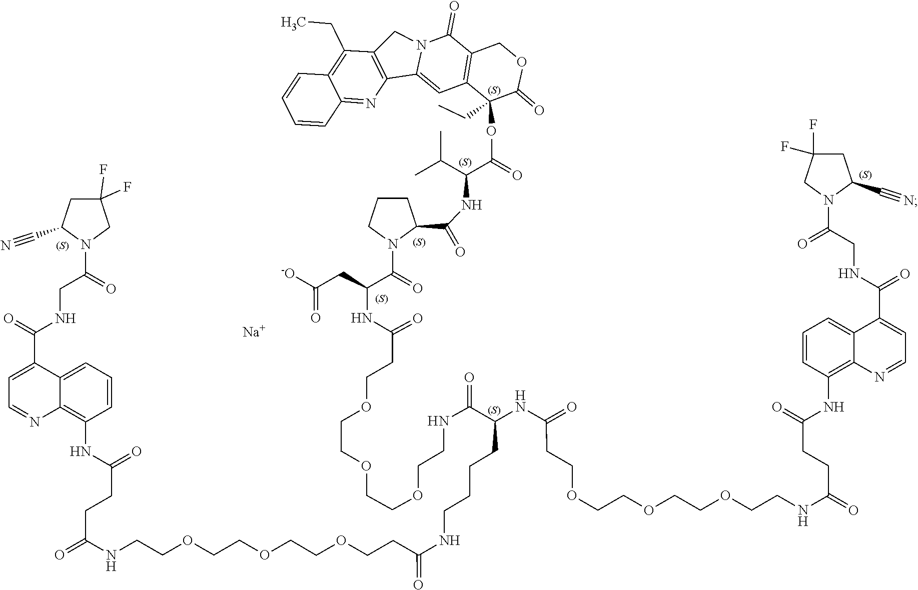
or a pharmaceutically acceptable salt thereof.

78. A compound having a structure of Formula (II), Formula (III), Formula (IV) Formula (V), or Formula (VII): 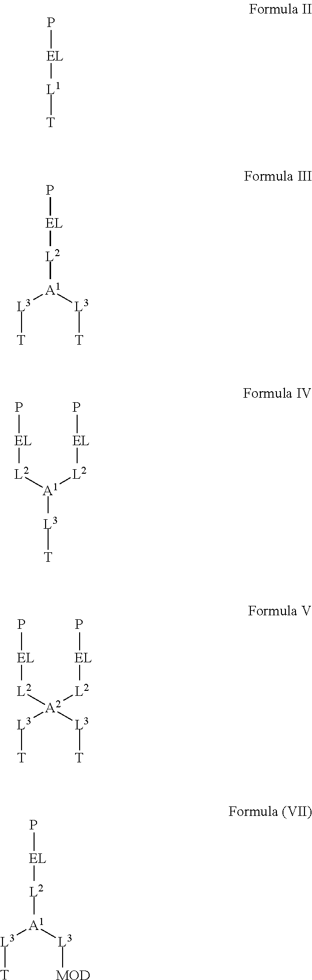
wherein:
each P is a tubulin polymerization inhibitor, a kinesin spindle protein inhibitor, a cyclin-dependent kinase inhibitor, an immunomodulator, an epidermal growth factor receptor inhibitor, or a microtubule inhibitor; preferably an auristatin, more preferably auristatin E, monomethyl auristatin E, or a derivative thereof, a kinesin spindle protein inhibitor, a cyclin-dependent kinase 9 inhibitor, a toll-like receptor 7 and/or 8 agonist, an epidermal growth factor receptor inhibitor, or a taxane, more preferably paclitaxel, or a derivative thereof;
each EL is a cleavable peptide linker, preferably cleaved by cathepsin B, legumain, or neutrophil elastase, more preferably by neutrophil elastase.
each L¹, L², and L³, is independently a bivalent linker,
A¹ is a trivalent linker,
A² is a tetravalent linker;
each T is a target protein binder, preferably a prostate-specific membrane antigen binder, carbonic anhydrase 9 binder, fibroblast activation protein binder, folate receptor binder, heat-shock protein 90 binder, an αvβ6 integrin binder, or an αvβ3 integrin binder or 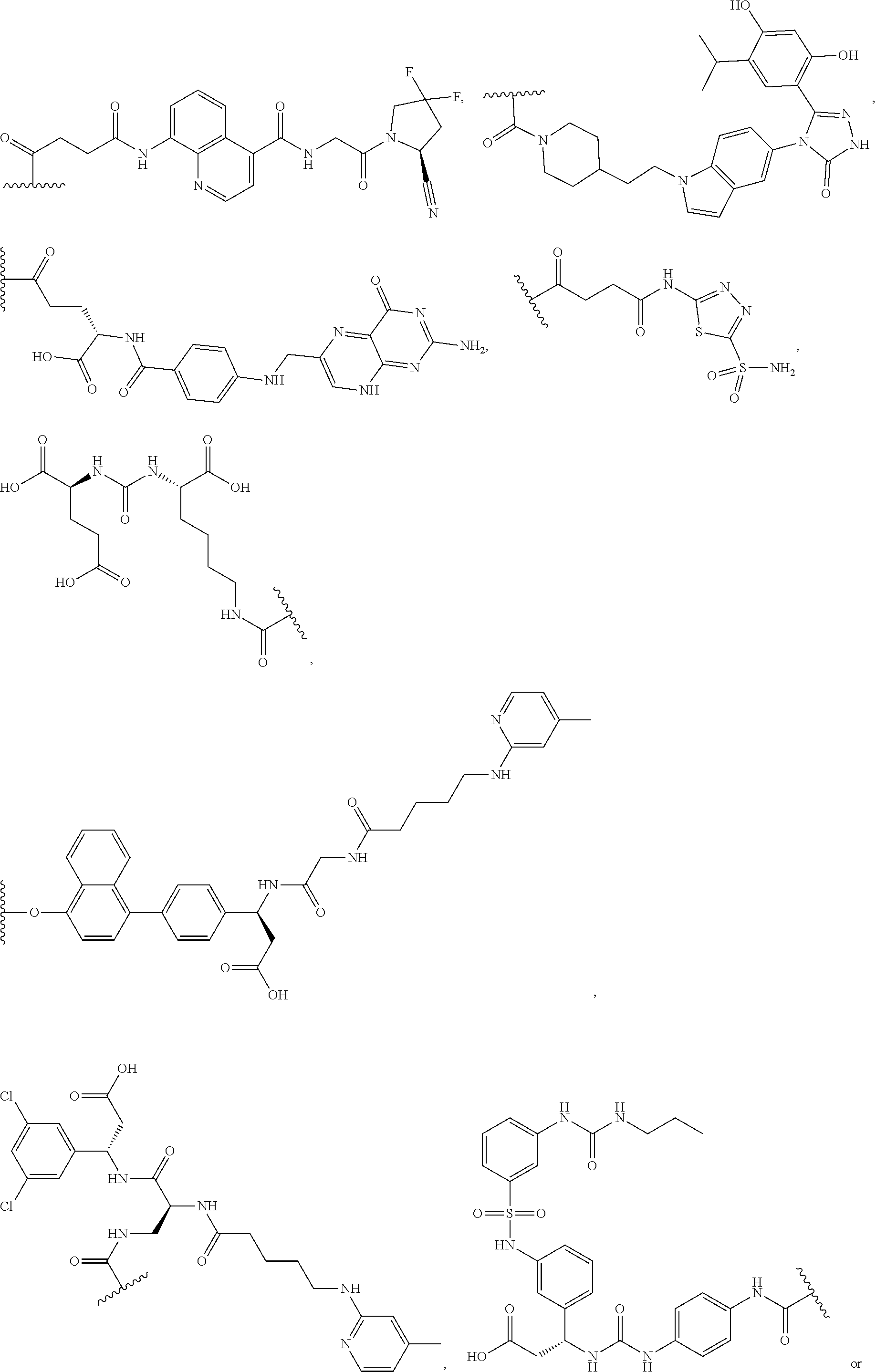 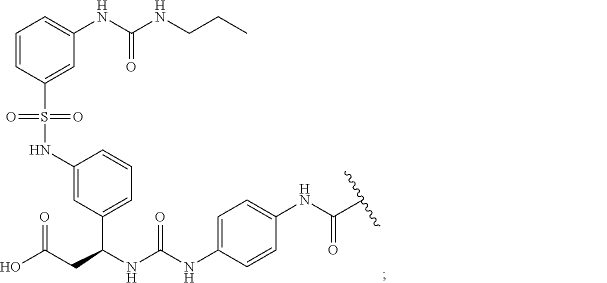 [SMILES: *C(=O)Nc1ccc(NC(=O)N[C@@H](CC(=O)O)c2cccc(NS(=O)(=O)c3cccc(NC(=O)NCCC)c3)c2)cc1]
or a pharmaceutically acceptable salt thereof; and
MOD is a pharmacokinetic modulating group,
preferably having the structure of Formula (II) or Formula (III): 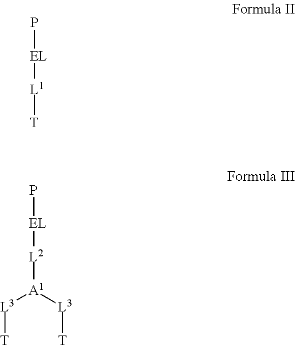
preferably having the structure: 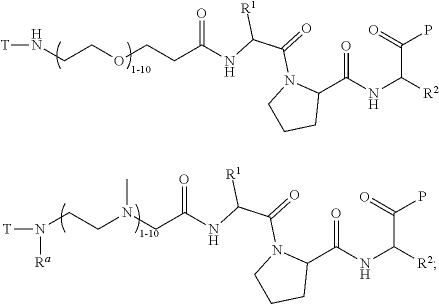
wherein:
P is an auristatin, a kinesin spindle protein inhibitor, a toll-like receptor 7 and/or 8 agonist, an epidermal growth factor receptor inhibitor, or a taxane;
R^{a} is hydrogen or —CH₃;
R¹ is hydrogen, —CH₃, —CH₂CH₃, —CH₂CH₂CH₃, —CH₂C(O)NH₂, or —CH₂C(O)OH;
R² is —CH₃, —CH(CH₃)₂, —CH₂CH(CH₃)₂, or —CH(CH₃)CH₂CH₃; and
T is a target protein binder.
preferably having the structure: 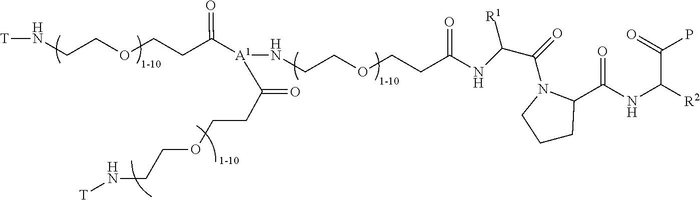
wherein:
P is an auristatin, a kinesin spindle protein inhibitor, a toll-like receptor 7 and/or 8 agonist, an epidermal growth factor receptor inhibitor, or a taxane;
R¹ is hydrogen, —CH₃, —CH₂CH₃, —CH₂CH₂CH₃, —CH₂C(O)NH₂, or —CH₂C(O)OH; and
R² is —CH₃, —CH(CH₃)₂, —CH₂CH(CH₃)₂, or —CH(CH₃)CH₂CH₃.

79. The compound of claim 78, or a pharmaceutically acceptable salt thereof, wherein P is: 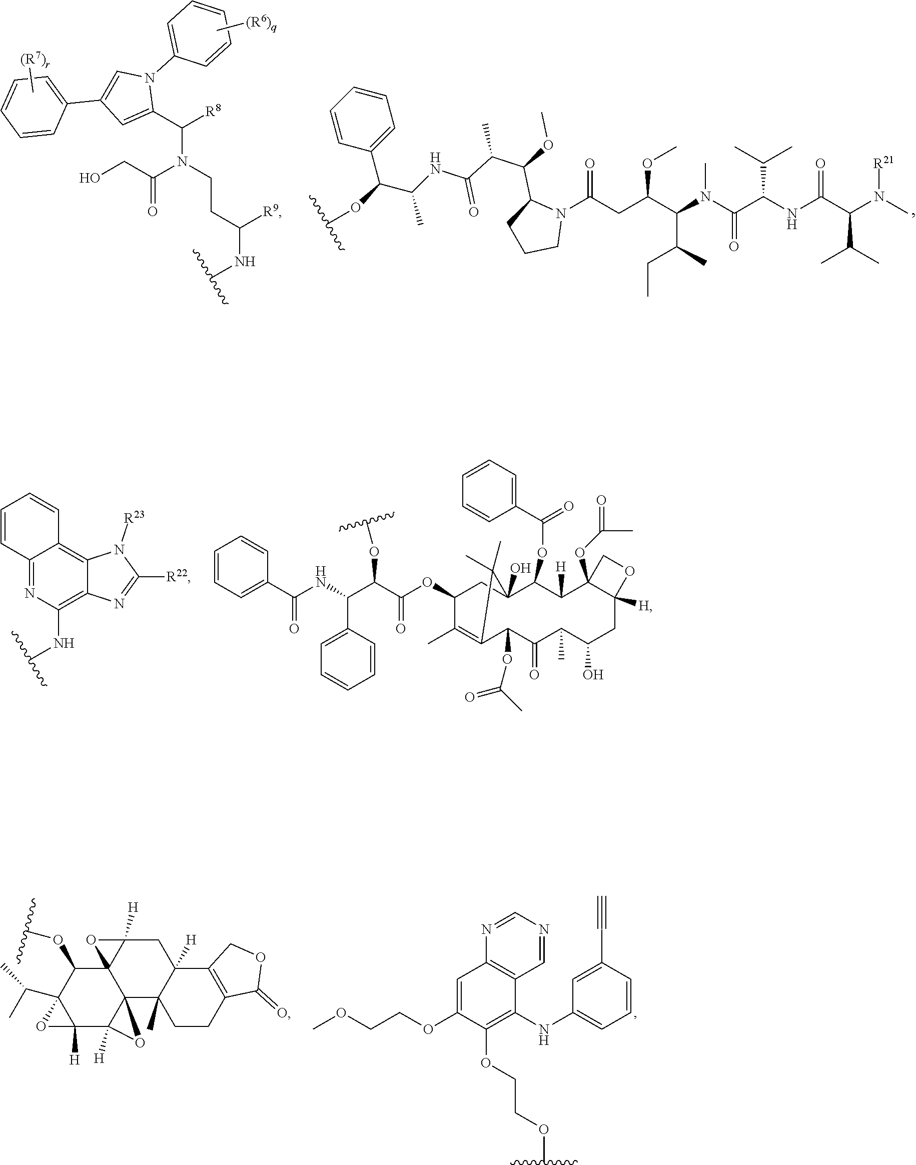 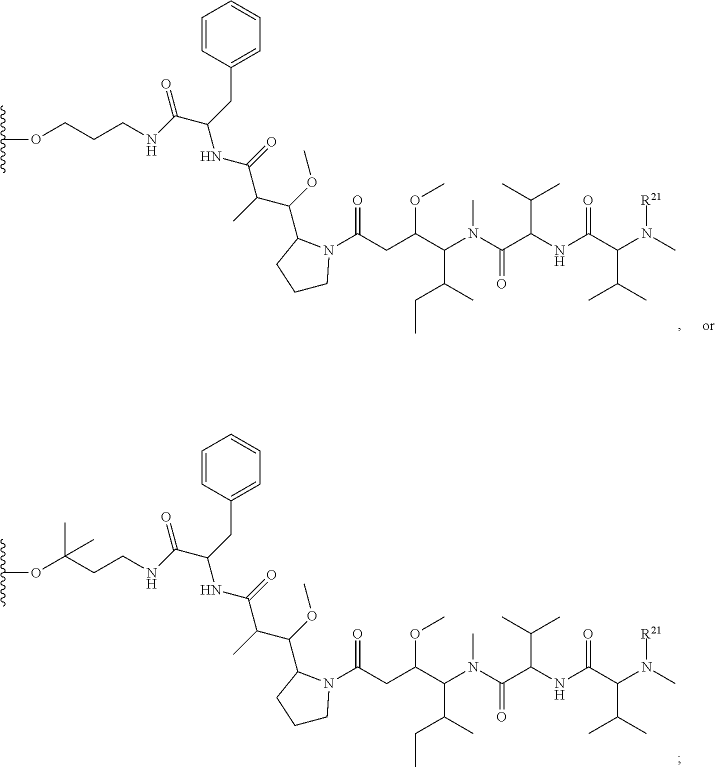
or a pharmaceutically acceptable salt thereof;
wherein:
each R⁶ and R⁷ is independently hydrogen, halogen, CN, —C₁₋₆ alkyl, or C₁₋₆ haloalkyl;
R⁸ is hydrogen, halogen, CN, —C₁₋₆ alkyl, C₁₋₆ haloalkyl, C₃₋₆ cycloalkyl, or 5- to 7-membered heterocycloalkyl;
R⁹ is hydrogen, halogen, CN, C₁₋₆ alkyl, —C(O)NH₂, —C(O)NHC₁₋₆ alkyl, —C(O)N(C₁₋₆ alkyl)₂, —C(O)NHC₁₋₆ alkyl-C(O)NHC₁₋₆ alkyl, —C(O)NHC₁₋₆ alkyl-NHC(O)C₁₋₆ alkyl, —NH₂, —NHC₁₋₆ alkyl, —N(C₁₋₆ alkyl)₂, —NHC(O)C₁₋₆ alkyl, —OH, or —OC₁₋₆ alkyl; wherein each C₁₋₆ alkyl is substituted with 0-5 R¹⁰;
R²¹ is hydrogen or C₁₋₆ alkyl;
R²² is hydrogen or C₁₋₆ alkyl, wherein the C₁₋₆ alkyl is unsubstituted or substituted with R²⁴;
R²³ is hydrogen, C₁₋₆ alkyl, or benzyl, wherein the C₁₋₆ alkyl or benzyl is unsubstituted or substituted with one, two, or three R² groups;
R²⁴ is —OH, —O(C₁₋₆ alkyl), —NH₂, —NH(C₁₋₆ alkyl), —N(C₁₋₆ alkyl)₂, —SH, or —S(C₁₋₆ alkyl);
R²⁵ is —OH, —O(C₁₋₆ alkyl), —NH₂, —NH(C₁₋₆ alkyl), —N(C₁₋₆ alkyl)₂, —NHS(O)₂(C₁₋₆ alkyl), C₁₋₆ alkyl, C₁₋₆ aminoalkyl, or OCH₂CH₂NHC(O)(C₁₋₆ aminoalkyl);
q is 0, 1, 2, 3, 4, or 5; and
r is 0, 1, 2, 3, 4, or 5 preferably wherein P is: 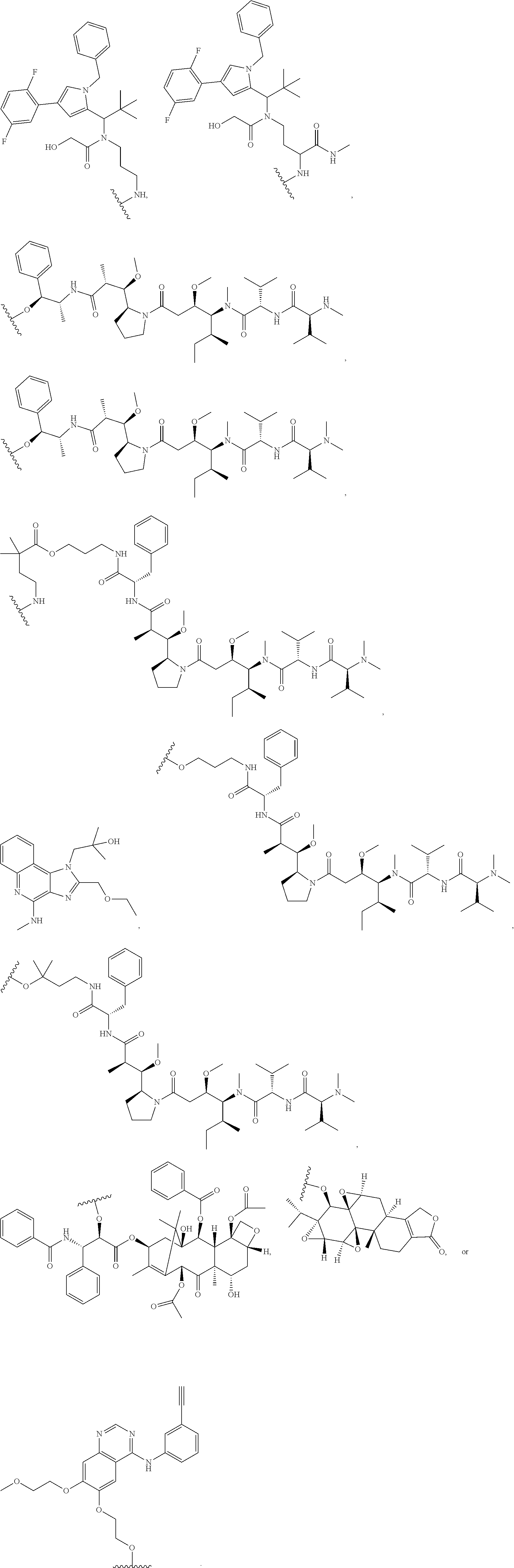
or a pharmaceutically acceptable salt thereof.

80. The compound of claim 79, or a pharmaceutically acceptable salt thereof, having the structure: 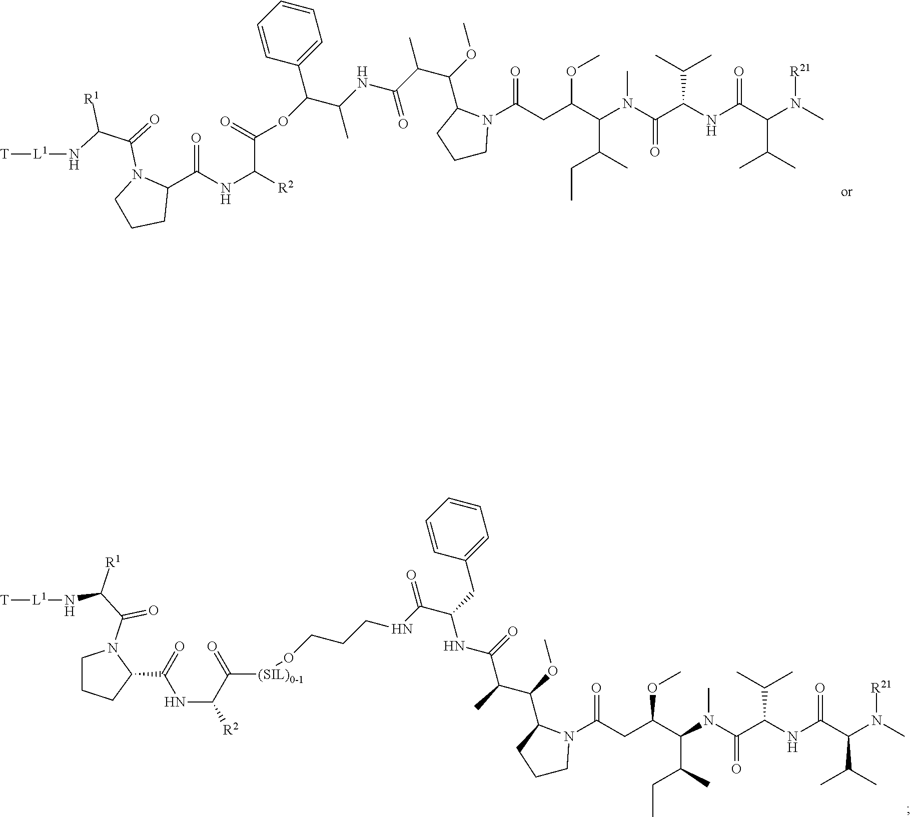
wherein:
T is a target protein binder;
L¹ is a bivalent linker,
R¹ is hydrogen, —CH₃, —CH₂CH₃, —CH₂CH₂CH₃, —CH₂C(O)NH₂, or —CH₂C(O)OH;
R² is —CH₃, —CH(CH₃)₂, —CH₂CH(CH₃)₂, or —CH(CH₃)CH₂CH₃;
R²¹ is hydrogen or —CH₃; and
SIL is a C₂₋₁₈ heteroalkylene group.

81. The compound of claim 79, or a pharmaceutically acceptable salt thereof, having the structure: 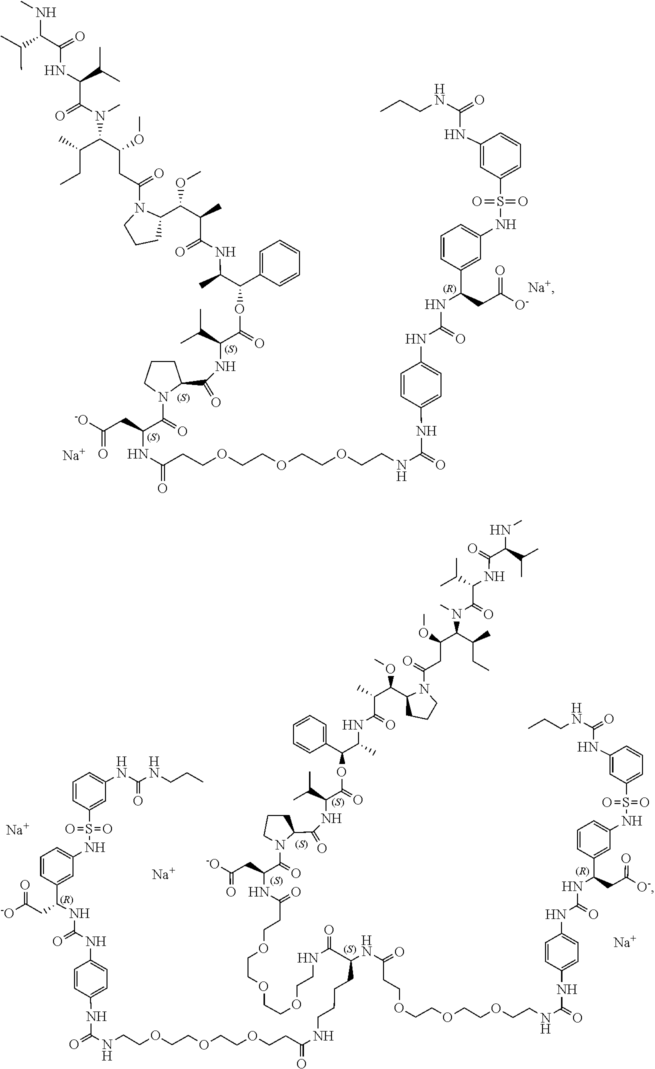 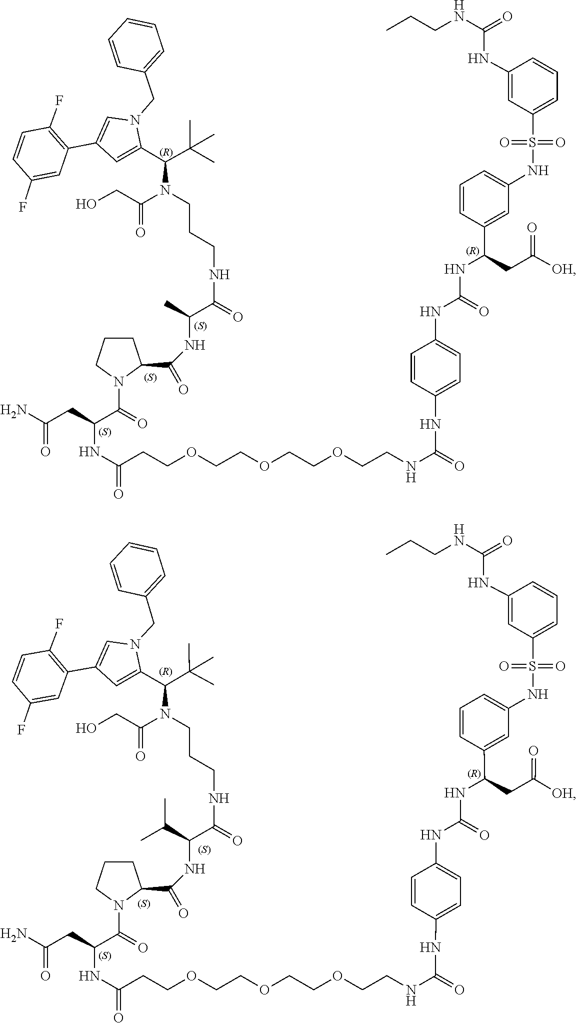 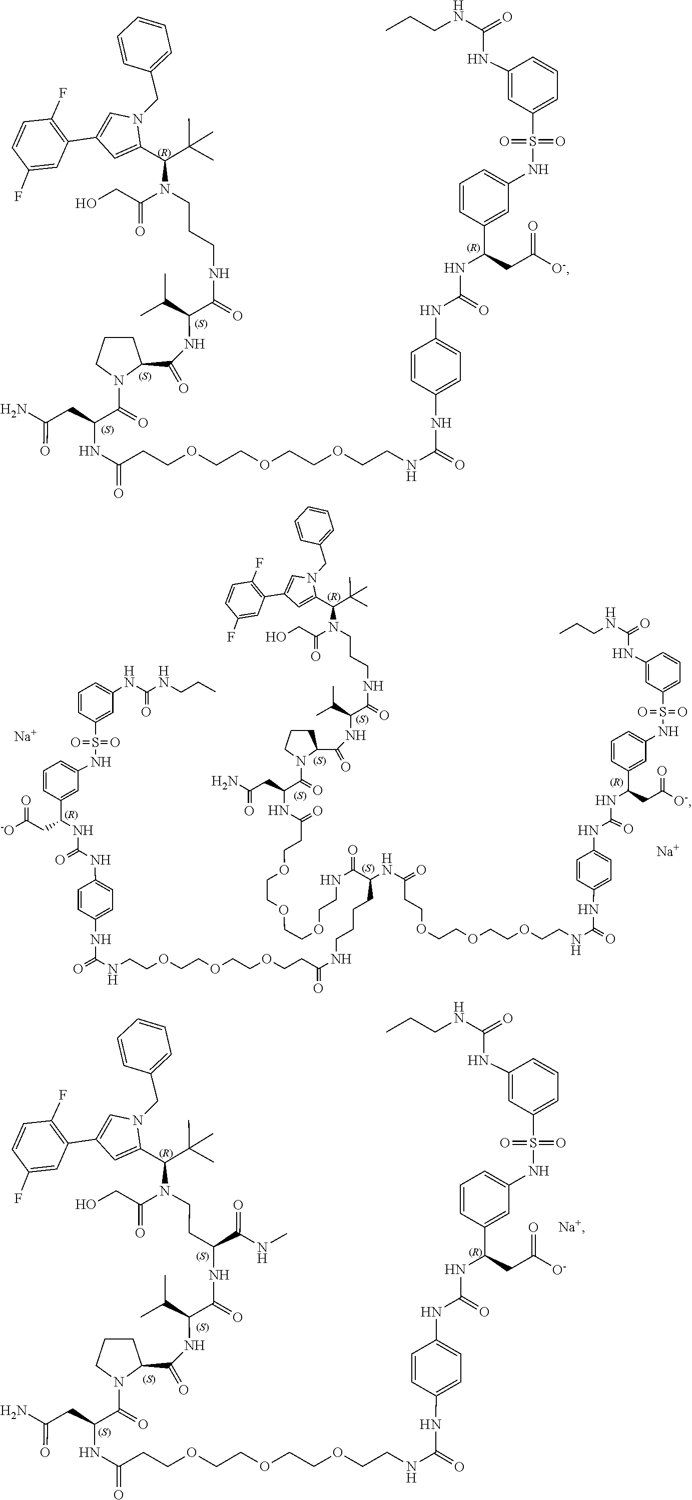 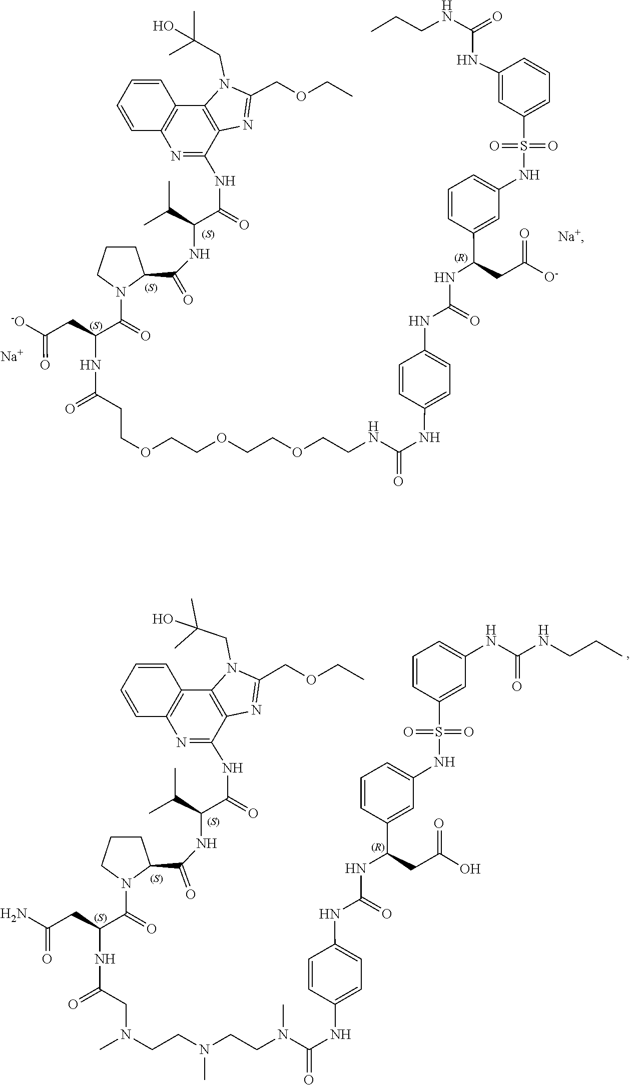 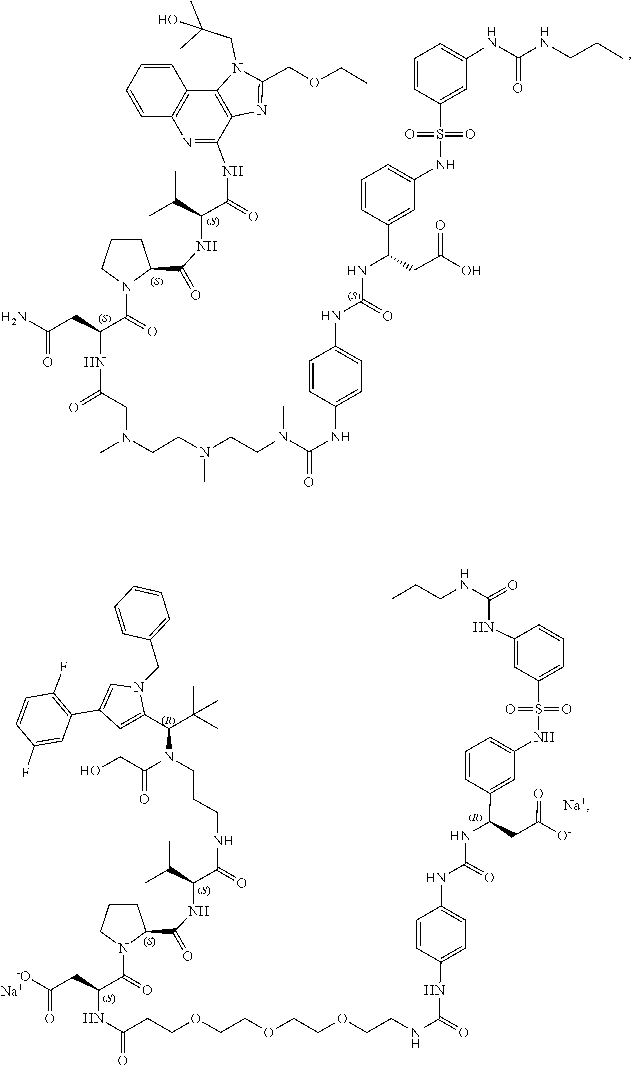 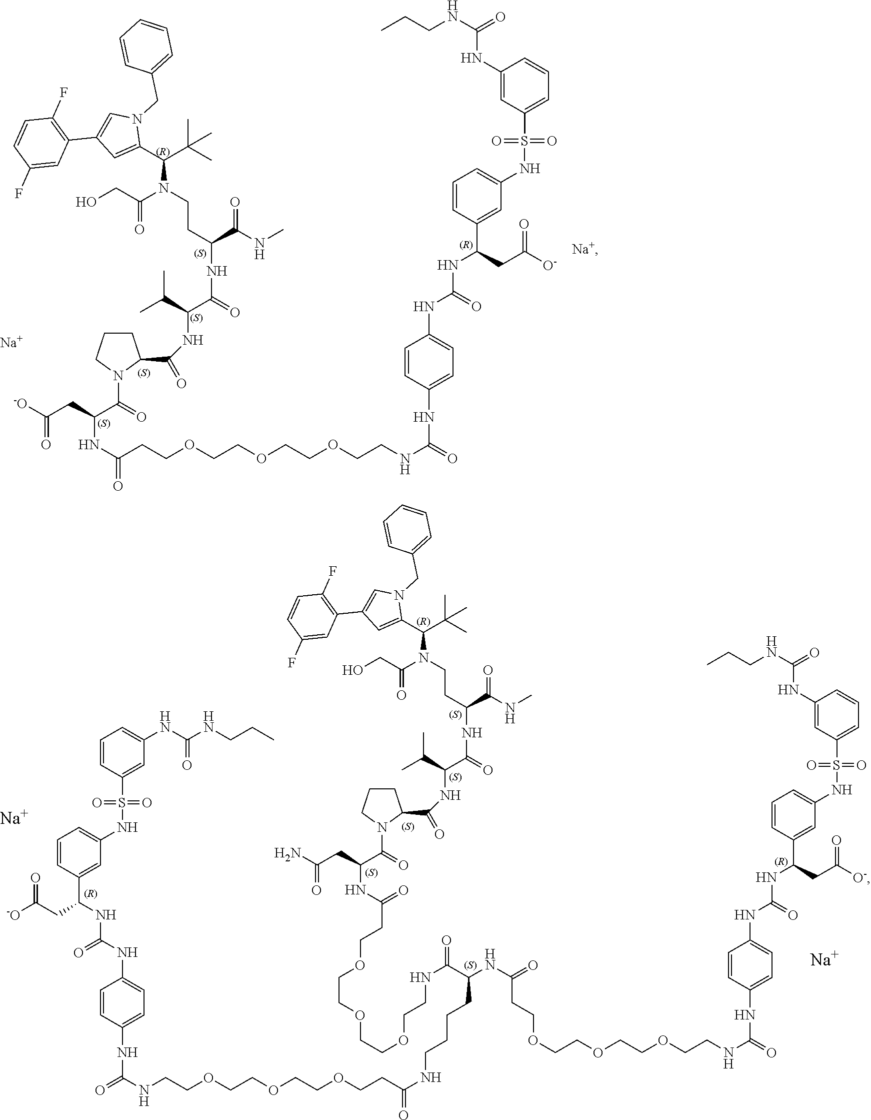 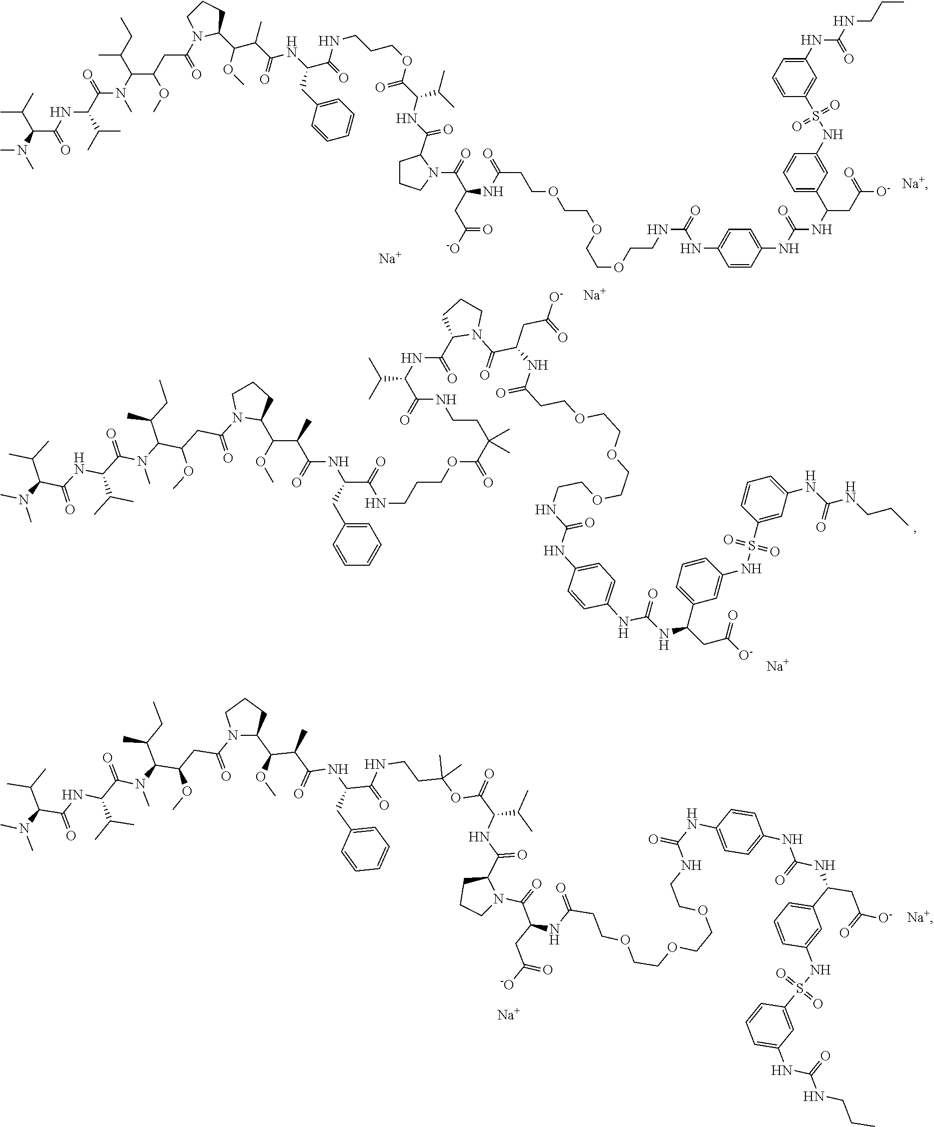 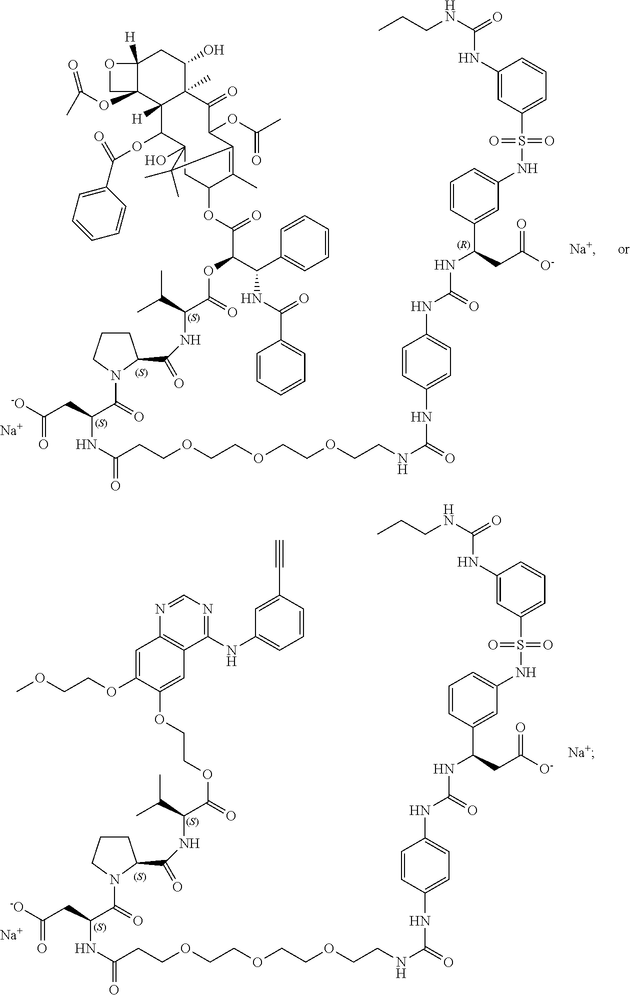
or a pharmaceutically acceptable salt thereof.

82. A compound, or a pharmaceutically acceptable salt thereof, having a structure Formula (IV), Formula (V), or Formula (VII): 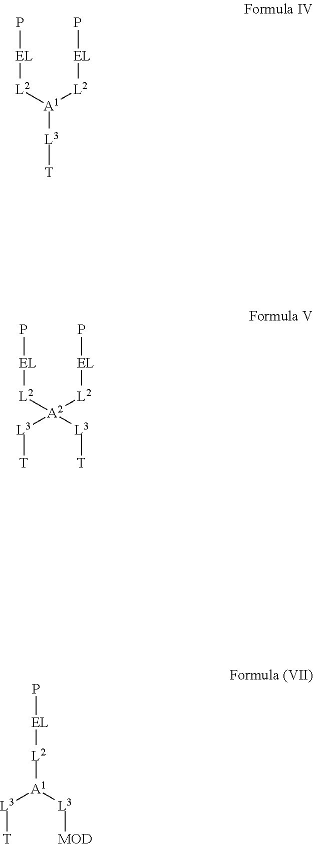
wherein:
each P is a payload; preferably a tubulin polymerization inhibitor, topoisomerase inhibitor, oxidative phosphorylation inhibitor, kinase inhibitor, dihydrofolate reductase inhibitor, histone deacetylase inhibitor, microtubule inhibitor, or an immuonomodulator, or a camptothecin or camptothecin derivative, an auristatin or auristatin derivative, a kinesin spindle protein inhibitor, a cyclin-dependent kinase 9 inhibitor, a toll-like receptor agonist, an epidermal growth factor receptor inhibitor, or a taxane;
each EL is a cleavable peptide linker, preferably cleaved by cathepsin B, legumain, or neutrophil elastase, more preferably by neutrophil elastase;
each L² and L³ is independently a bivalent linker,
A¹ is a trivalent linker,
A² is a tetravalent linker;
each T is a target protein binder, preferably a prostate-specific membrane antigen binder, carbonic anhydrase 9 binder, fibroblast activation protein binder, folate receptor binder, heat-shock protein 90 binder, an αvβ6 integrin binder, or an αvβ3 integrin binder, and
MOD is a pharmacokinetic modulating group.
having preferably the structure: 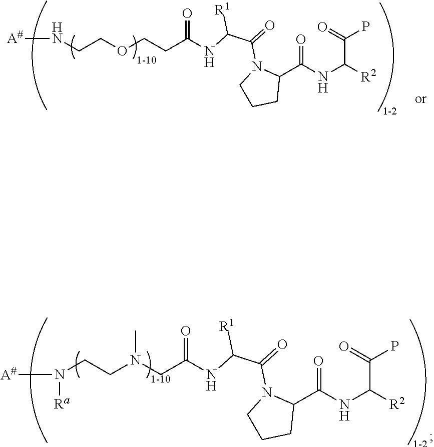
wherein:
A* represents A¹ or A²
R^{a} is hydrogen or —CH₃;
R¹ is hydrogen, —CH₃, —CH₂CH₃, —CH₂CH₂CH₃, —CH₂C(O)NH₂, or —CH₂C(O)OH;
R² is —CH₃, —CH(CH₃)₂, —CH₂CH(CH₃)₂, or —CH(CH₃)CH₂CH₃;
R²¹ is hydrogen or —CH₃; and
P is a camptothecin or camptothecin derivative, an auristatin or auristatin derivative, a kinesin spindle protein inhibitor, a cyclin-dependent kinase 9 inhibitor, a toll-like receptor agonist, an epidermal growth factor receptor inhibitor, or a taxane.

83. The compound of claim 82, or a pharmaceutically acceptable salt thereof, wherein each P is: 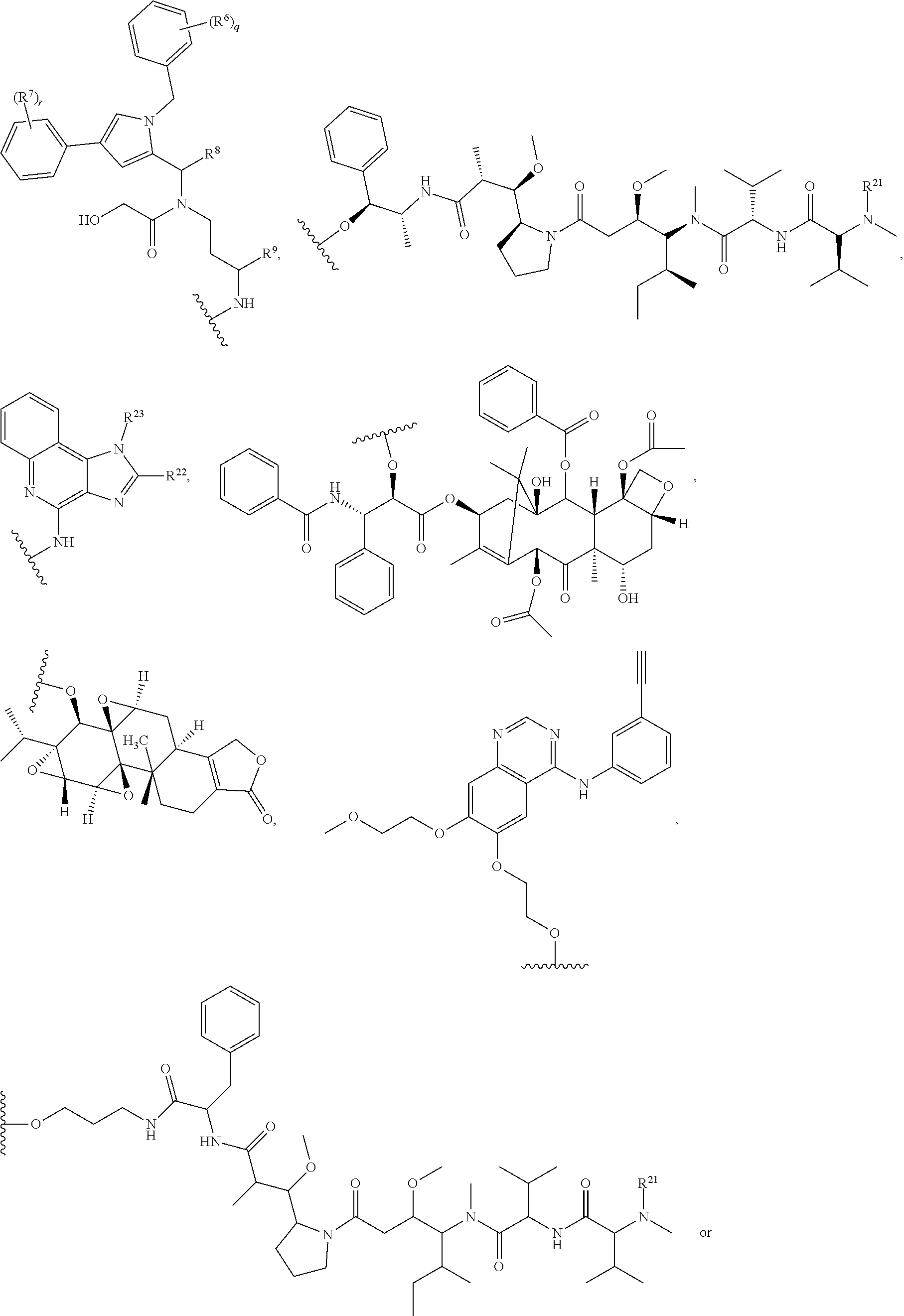 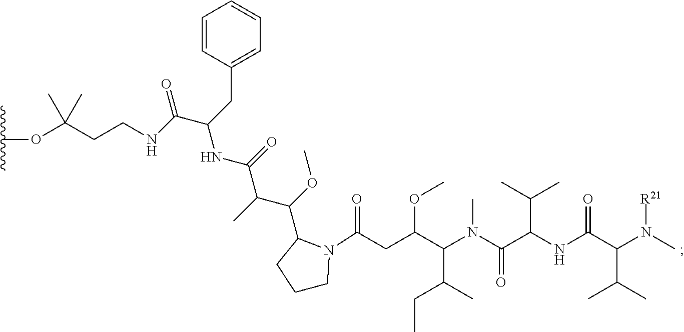
or a pharmaceutically acceptable salt thereof;
wherein:
each R⁶ and R⁷ is independently hydrogen, halogen, CN, —C₁₋₆ alkyl, or C₁₋₆ haloalkyl;
R⁸ is hydrogen, halogen, CN, —C₁₋₆ alkyl, C₁₋₆ haloalkyl, C₃₋₆ cycloalkyl, or 5- to 7-membered heterocycloalkyl;
R⁹ is hydrogen, halogen, CN, C₁₋₆ alkyl, —C(O)NH₂, —C(O)NHC₁₋₆ alkyl, —C(O)N(C₁₋₆ alkyl)₂, —C(O)NHC₁₋₆ alkyl-C(O)NHC₁₋₆ alkyl, —C(O)NHC₁₋₆ alkyl-NHC(O)C₁₋₆ alkyl, —NH₂, —NHC₁₋₆ alkyl, —N(C₁₋₆ alkyl)₂, —NHC(O)C₁₋₆ alkyl, —OH, or —OC-6 alkyl; wherein each C₁₋₆ alkyl is substituted with 0-5 R¹⁰;
R²¹ is hydrogen or C₁₋₆ alkyl;
R²² is hydrogen or C₁₋₆ alkyl, wherein the C₁₋₆ alkyl is unsubstituted or substituted with R²⁴;
R²³ is hydrogen, C₁₋₆ alkyl, or benzyl, wherein the C₁₋₆ alkyl or benzyl is unsubstituted or substituted with one, two, or three R² groups;
R²⁴ is —OH, —O(C₁₋₆ alkyl), —NH₂, —NH(C₁₋₆ alkyl), —N(C₁₋₆ alkyl)₂, —SH, or —S(C₁₋₆ alkyl);
R²⁵ is —OH, —O(C₁₋₆ alkyl), —NH₂, —NH(C₁₋₆ alkyl), —N(C₁₋₆ alkyl)₂, —NHS(O)₂(C₁₋₆ alkyl), C₁₋₆ alkyl, C₁₋₆ aminoalkyl, or OCH₂CH₂NHC(O)(C₁₋₆ aminoalkyl);
q is 0, 1, 2, 3, 4, or 5; and
r is 0, 1, 2, 3, 4, or 5.

84. The compound of claim 82, or a pharmaceutically acceptable salt thereof, wherein each T is: 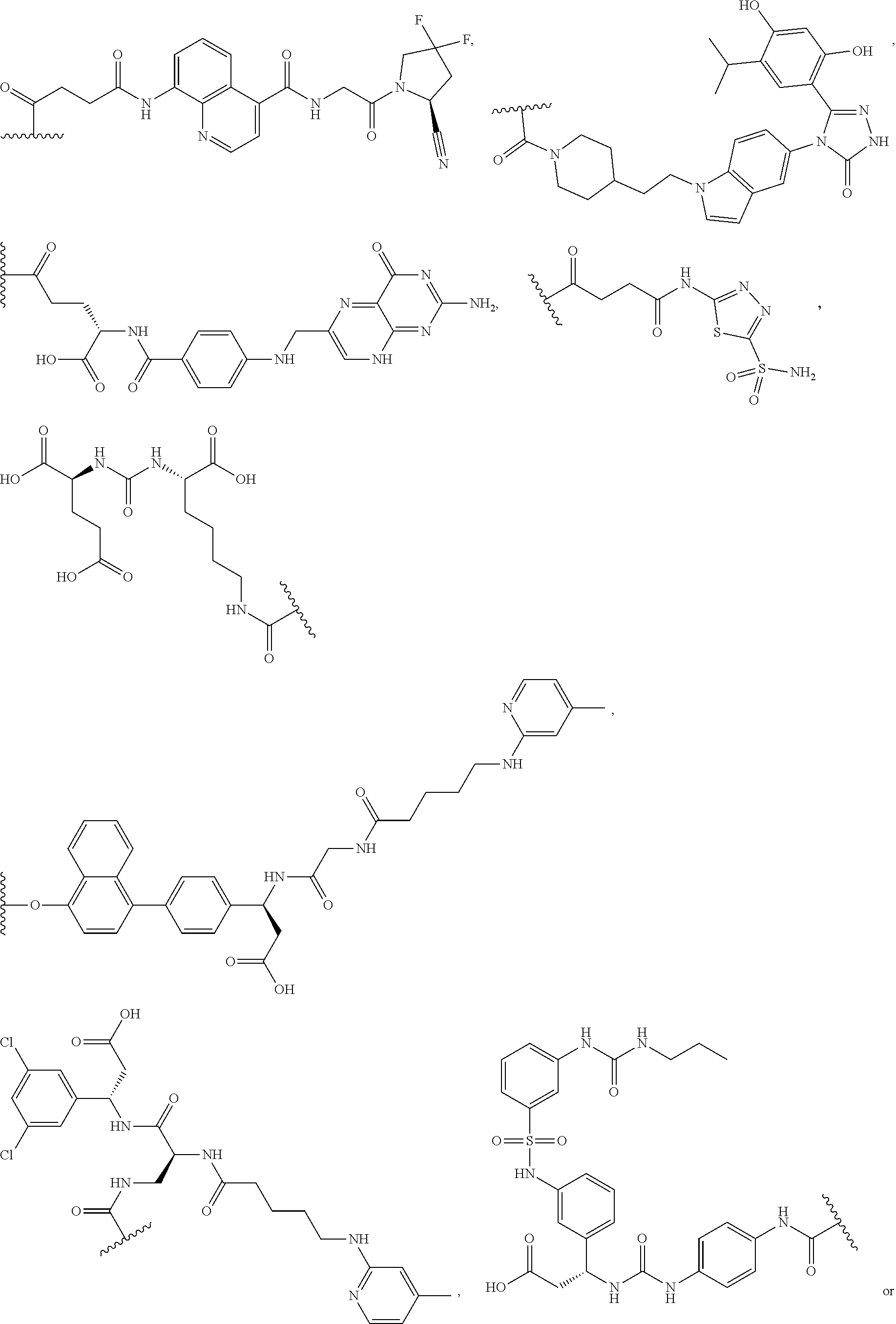 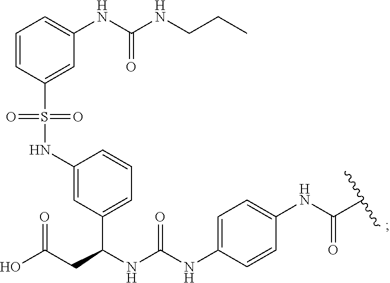 [SMILES: *C(=O)Nc1ccc(NC(=O)N[C@@H](CC(=O)O)c2cccc(NS(=O)(=O)c3cccc(NC(=O)NCCC)c3)c2)cc1]
or a pharmaceutically acceptable salt thereof.

85. The compound of claim 82, or a pharmaceutically acceptable salt thereof, wherein A¹ comprises an amino acid (e.g., lysine, glutamine, or glutamate), wherein the amino acid forms a bond with each L² and L³, optionally bridged via a C₁₋₁₂ alkyl or C₁₋₁₂ heteroalkyl linking arm; and/or
wherein A² comprises one or two amino acids (e.g., a lysine, a glutamine, glutamate, or a combination thereof), wherein the amino acid forms a bond with each L² and L³, optionally bridged via a C₁₋₁₂ alkylene or C₁₋₁₂ heteroalkylene linking arm; and/or
wherein A¹ is: 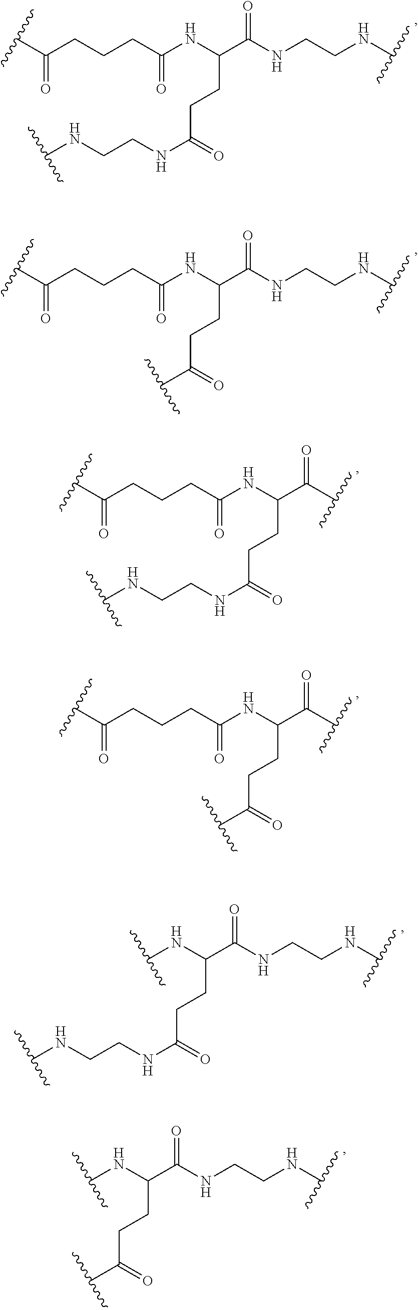 [SMILES: *NCCNC(=O)CCC(NC(=O)CCCC(*)=O)C(=O)NCCN* ; *NCCNC(=O)C(CCC(*)=O)NC(=O)CCCC(*)=O ; *NCCNC(=O)CCC(NC(=O)CCCC(*)=O)C(*)=O ; *C(=O)CCCC(=O)NC(CCC(*)=O)C(*)=O ; *NCCNC(=O)CCC(N*)C(=O)NCCN* ; *NCCNC(=O)C(CCC(*)=O)N*] 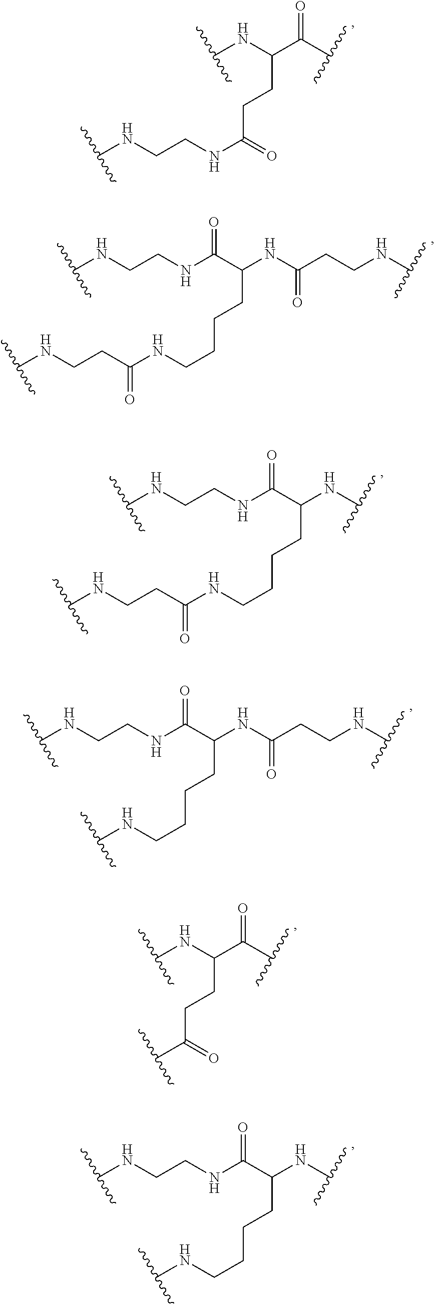 [SMILES: *NCCNC(=O)CCC(N*)C(*)=O ; *NCCNC(=O)C(CCCCNC(=O)CCN*)NC(=O)CCN* ; *NCCNC(=O)C(CCCCNC(=O)CCN*)N* ; *NCCCCC(NC(=O)CCN*)C(=O)NCCN* ; *NC(CCC(*)=O)C(*)=O ; *NCCCCC(N*)C(=O)NCCN*] 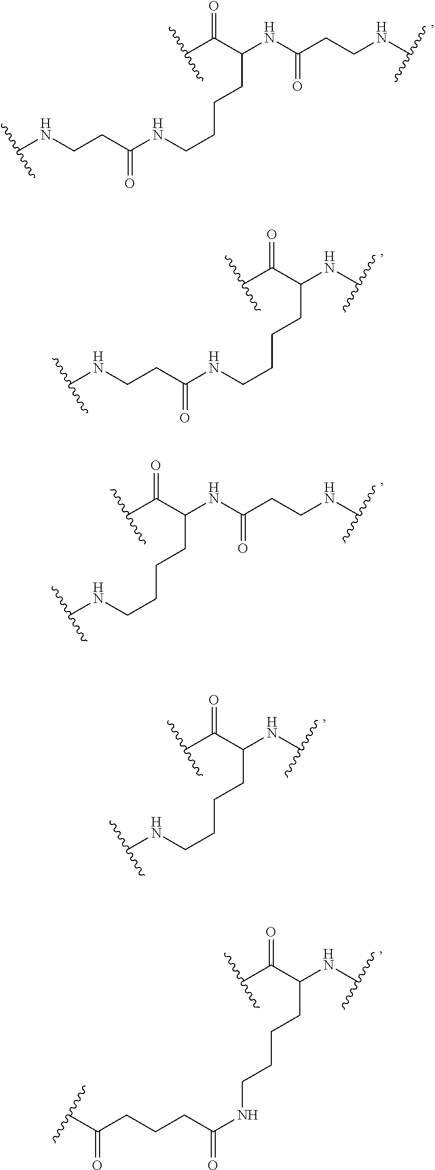 [SMILES: *NCCC(=O)NCCCCC(NC(=O)CCN*)C(*)=O ; *NCCC(=O)NCCCCC(N*)C(*)=O ; *NCCCCC(NC(=O)CCN*)C(*)=O ; *NCCCCC(N*)C(*)=O ; *NC(CCCCNC(=O)CCCC(*)=O)C(*)=O] 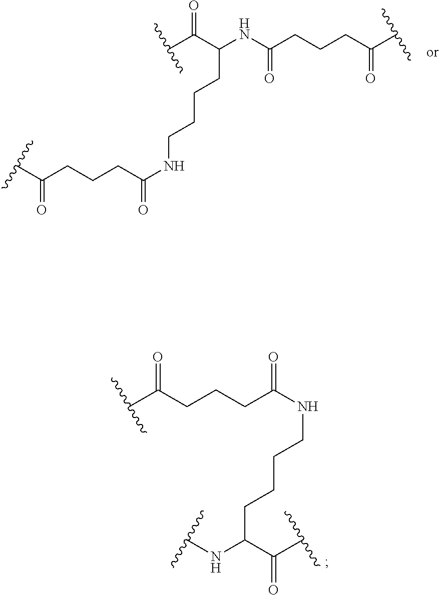 [SMILES: *C(=O)CCCC(=O)NCCCCC(NC(=O)CCCC(*)=O)C(*)=O ; *NC(CCCCNC(=O)CCCC(*)=O)C(*)=O]
A² is 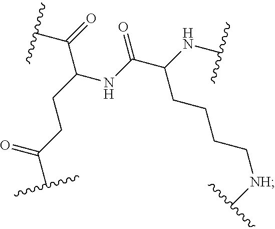 [SMILES: *NCCCCC(N*)C(=O)NC(CCC(*)=O)C(*)=O]
and
MOD is —COOH.

86. The compound of claim 82, or a pharmaceutically acceptable salt thereof, having the structure: 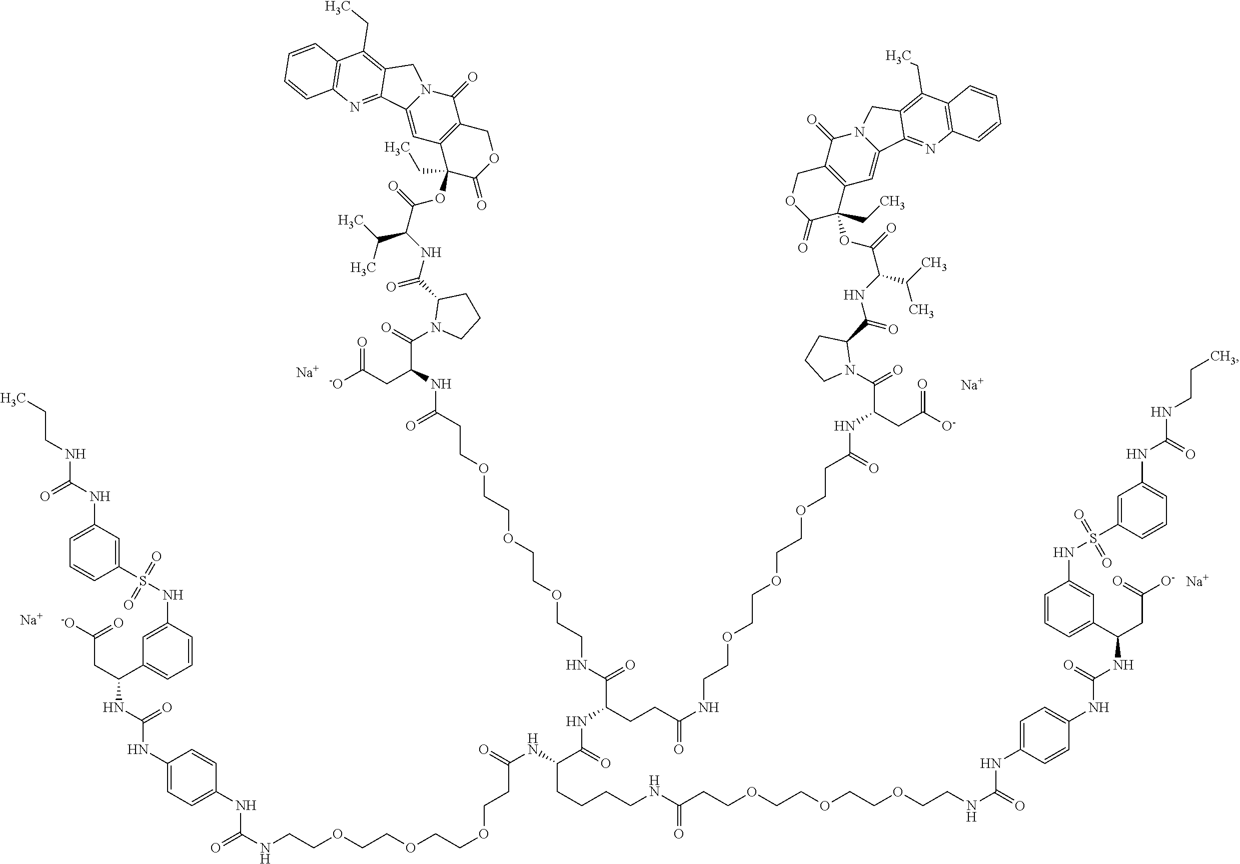 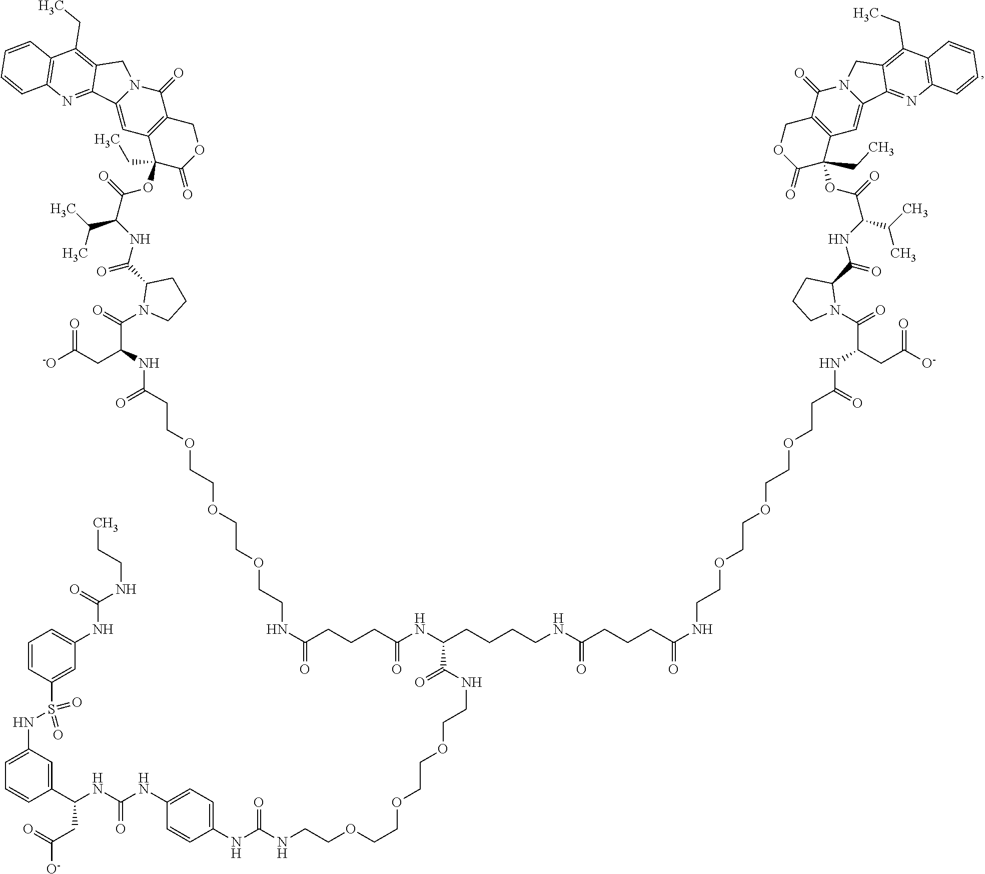 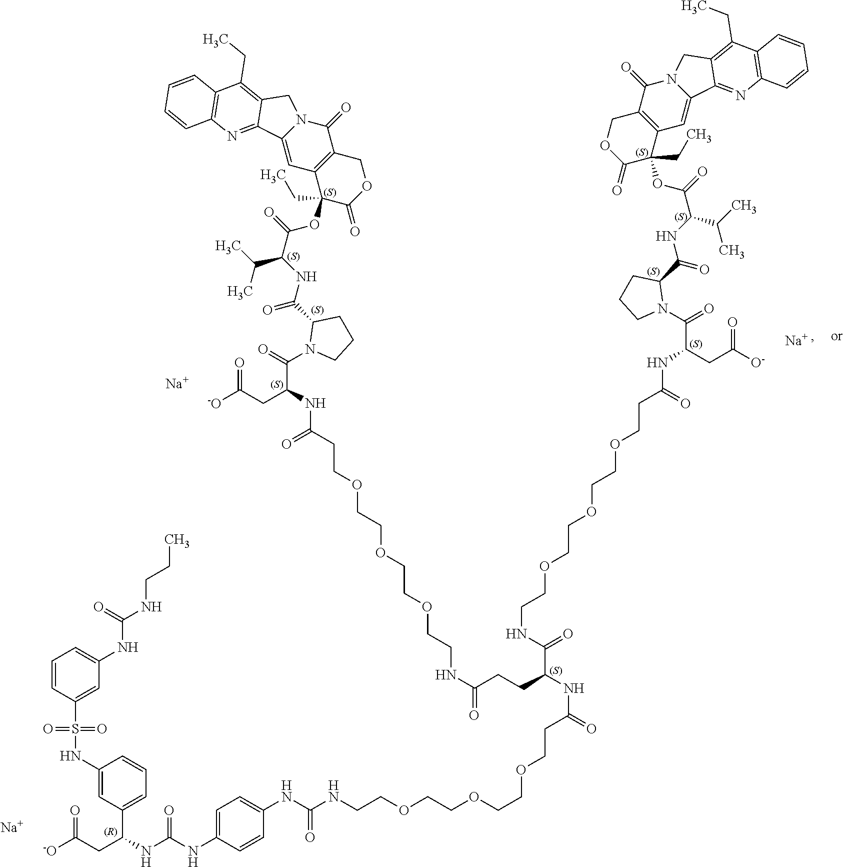 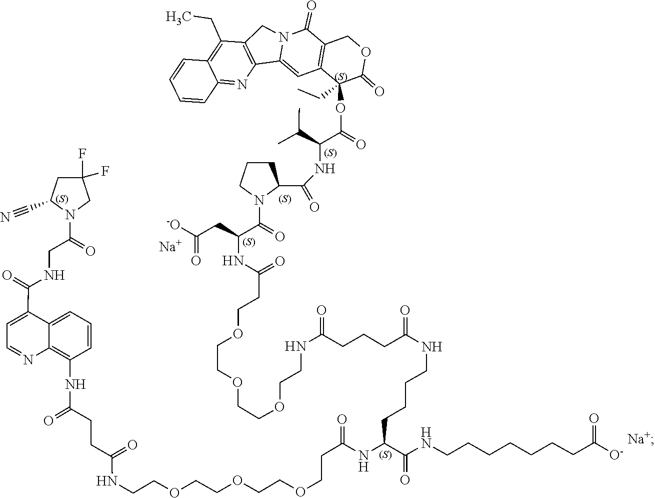
or a pharmaceutically acceptable salt thereof.

87. The compound of claim 75, or a pharmaceutically acceptable salt thereof, wherein each L¹, L², and L³ is a bivalent linker having a structure represented by formula: 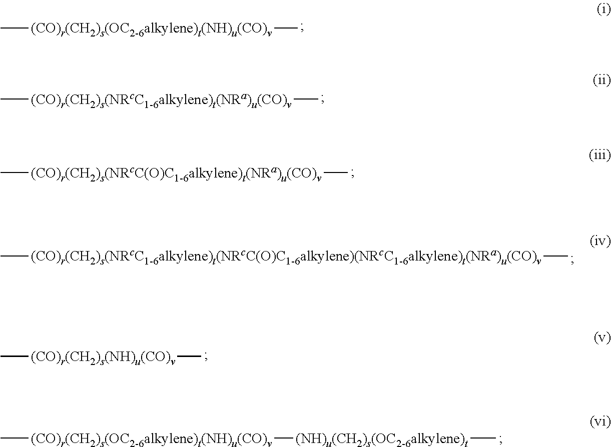
wherein:
R^{a} is, in each instance, independently selected from hydrogen or C₁₋₃ alkyl;
R^{c} is, in each instance, independently selected from hydrogen or C₁₋₃ alkyl;
r is 0 or 1; s is 0 to 10; t is 1 to 10; u is 0 or 1; and v is 0 or 1; and/or
wherein each L¹, L², and L is a bivalent linker having a structure represented by formula: 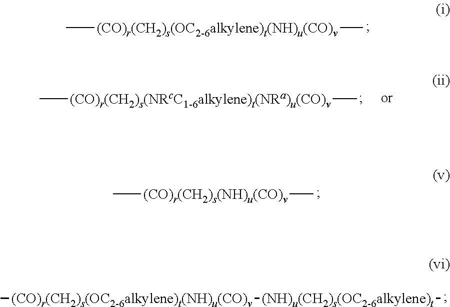
wherein each R^{a} and R^{c} is independently hydrogen or —CH₃;
r is 0 or 1;
s is 1 to 4;
t is 1 to 10;
u is 0 or 1; and
v is 0 or 1; and/or
wherein each L¹, L², and L³ is independently a bivalent linker of the formula: 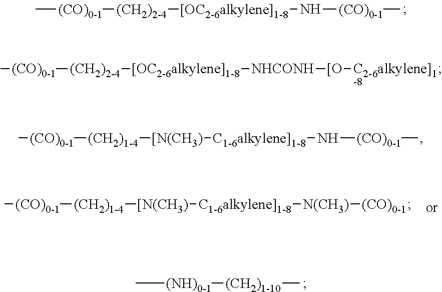
and/or
wherein:
each L¹ is: —CO—(CH₂)₂₋₄—[OCH₂CH₂]₁₋₈—NHCONH—[OCH₂CH₂]₁₋₈, —CO—(CH₂)₂₋₄—(OCH₂CH₂)₁₋₈—NH—, —CO—(CH₂)₂₋₄—(N(CH₃)CH₂CH₂)₁₋₈—N(CH₃)—, or —CO—(CH₂)₂₋₄ (N(CH₃)CH₂CH₂)₁₋₈—NH—;
each L² is: —CO—(CH₂)ₛ—(OCH₂CH₂)₁₋₈—NH—; and
each L³ is: —CO—(CH₂)ₛ—(OCH₂CH₂)₁₋₈—NH— or —NH—(CH₂)₁₋₁₀; and/or
wherein each L¹, L², and L³ is a bivalent linker comprising 2 to 20 polyethylene glycol groups.

88. A pharmaceutical composition comprising the compound of claim 75, or a pharmaceutically acceptable salt thereof; or a stereoisomer or mixture of stereoisomers thereof; and at least one pharmaceutically acceptable excipient.

89. The compound of claim 75, or a pharmaceutically acceptable salt thereof; or a stereoisomer or mixture of stereoisomers thereof; for use in treating a hyperproliferative disorder, preferably
an autoimmune disorder, or
a cancer, preferably
a solid tumor,
a hematological malignancy,
a B-cell malignancy,
a MYC-driven cancer,
a MCL1-driven cancer,
a tumor overexpressing MYC, MYB or MCL1 mRNA; or MYC, MCL1, or MYB proteins associated therewith,
a transcriptionally addicted tumor,
aggressive non-Hodgkin lymphoma (NHL), double-hit diffuse large B-cell lymphoma (DH-DLBCL), high grade B-cell lymphoma (HGBCL), transformed follicular lymphoma (FL), mantle cell lymphoma (MCL), chronic lymphocytic leukemia (CLL), small lymphocytic lymphoma (SLL), or Richter syndrome (RS),
relapsed/refractory (r/r) aggressive non-Hodgkin lymphoma (r/r NHL), relapsed/refractory double-hit diffuse large B-cell lymphoma (r/r DH-DLBCL), relapsed/refractory high grade B-cell lymphoma (r/r HGBCL), relapsed/refractory transformed follicular lymphoma (r/r FL), relapsed/refractory mantle cell lymphoma (r/r MCL), relapsed/refractory chronic lymphocytic leukemia (r/r CLL), relapsed/refractory small lymphocytic lymphoma (r/r SLL), or relapsed/refractory Richter syndrome (r/r RS),
ovarian cancer, breast cancer, or prostate cancer,
advanced ovarian cancer, triple negative breast cancer, or castration-resistant neuroendocrine prostate cancer,
neuroblastoma, or osteosarcoma, or
or an ophthalmic condition disorder, preferably
macular degeneration; or
a cardiovascular condition disorder, preferably
cardiac hypertrophy.
